(19) United States
(12) Patent Application Publication
(10) Pub. No.: US 2010/0216655 A1
(43) Pub. Date: Aug. 26, 2010

(54) SEQUENCE VARIANTS FOR INFERRING HUMAN PIGMENTATION PATTERNS

(76) Inventor: Patrick SULEM, Reykjavik (IS)

Correspondence Address: MARSHALL, GERSTEIN & BORUN LLP, 233 SOUTH WACKER DRIVE, 6300 WILLIS TOWER, CHICAGO, IL, 60606-6357, US

(21) Appl. No.: 12/682,587

(22) PCT Filed: Oct. 13, 2008
(86) PCT No.: PCT/IS2008/000017

§ 371 Date: Apr. 9, 2010

(30) Foreign Application Priority Data
Oct. 12, 2007 (IS) 8683
May 16, 2008 (IS) 8731

(51) Int. Cl. C40B 30/02; C40B 30/00; C12Q 1/68; C40B 40/06; G06F 9/30

(52) U.S. Cl. 506/8; 506/7; 435/6; 506/16; 712/220; 712/E09.016

(57) ABSTRACT

The present invention discloses variants that are predictive of human pigmentation patterns. The invention furthermore relates to variants that are useful for determining risk of skin cancer, including melanoma and basal cell carcinoma. The disclosed variants can be utilized for the determination of the natural pigmentation patterns of a human individual, and for determining a susceptibility to melanoma and basal cell carcinoma, from a sample of genetic material. Methods and kits including the variants described are useful in e.g. forensic testing and diagnostic applications.

### INTRODUCTION

Hair, eye and skin pigmentation are among the most easily visible examples of human phenotypic variation and have a large normal range in humans. Pigmentation is dependent upon the amount and type of the light-absorbing polymer melanin produced within ocular, epidermal and follicular melanocytes. Hair colour is determined by the melanin granules deposited into the hair shaft and eye colour by melanin composition in the anterior border layer of the iris. In the skin, melanin is produced by melanocytes, which are found in the epidermis.

It has long been known that visible traits have a genetic component. The fact that pigmentation is a heritable trait was recognized and assessed as early as the 19^{th} century by Galton (Galton, F. Nature 34, 137 (1886)) and since then a high degree of heritability of hair and eye colour has been consistently demonstrated (Posthuma, D. et al. Behav Genet 36, 12-7 (2006), Brauer, G. & Chopra, V. P. Anthropol Anz 36, 109-20 (1978)). More recently, other forms of human pigmentation characteristics, such as skin sensitivity to radiation from the sun, freckle count and nevi count have also been shown to be highly heritable (Bataille, V., Snieder, H., MacGregor, A. J., Sasieni, P. & Spector, T. D. J Natl Cancer Inst 92, 457-63 (2000)).

Linkage studies on hair colour and eye colour have revealed strong linkage to a region on chromosome 15 encompassing the pink eye dilution gene (OCA2), which has previously been linked to albinism (cite), to brown eye and brown hair (Posthuma, D. et al. Behav Genet 36, 12-7 (2006), Eiberg, H. & Mohr, J. Clin Genet 32, 125-8 (1987), Eiberg, H. & Mohr, J. Eur J Hum Genet 4, 237-41 (1996)). Coding and non coding variants in OCA2 have since been associated with variation of eye colour (blue versus brown) and hair colour (dark versus light shade) and fair skin (Frudakis, T. et al. Genetics 165, 2071-83 (2003), Sturm, R. A. & Frudakis, T. N. Trends Genet 20, 327-32 (2004), Duffy, D. L. et al. Am J Hum Genet 80, 241-52 (2007)).

More than 100 genes affecting pigmentation have been cloned in mice, and about 60 human homologues of these genes have been described and are candidates for affecting pigmentation variability in humans (Hoekstra, H. E. Heredity 97, 222-34 (2006)). The melanocortin 1 receptor (MC1R) was identified through such candidacy and multiple coding variants are established to cause red hair, fair skin, freckles, and associate with a poor tanning response and a skin cancer risk (Valverde, P., Healy, E., Jackson, I., Rees, J. L. & Thody, A. J. Nat Genet 11, 328-30 (1995), Rees, J. L. Am J Hum Genet 75, 739-51 (2004), Makova, K. & Norton, H. Peptides 26, 1901-8 (2005)). Other animals, including zebra fish have helped to identify candidate pigmentation genes in humans like the SLC24A5 gene (Lamason, R. L. et al. Science 310, 1782-6 (2005)) which has been associated with the golden phenotype in zebra fish. In humans opposite alleles of rs1426654 are fixated in Europeans versus non Europeans (Izagirre, N., Garcia, I., Junquera, C., de la Rua, C. & Alonso, S. Mol Biol Evol 23, 1697-706 (2006)) and lighter skin pigmentation was correlated with the number of copy of the “European” allele of rs1426654 (Lamason, R. L. et al. Science 310, 1782-6 (2005)).

Recently, a haplotype map of the human genome was published (Nature 437, 1299-320 (2005)) providing information on millions of SNPs distributed over whole the genome in four different populations (Caucasians, Africans, Chinese and Japanese), which allowed the detection of ethnicity informative markers and signs of selective pressure (Voight, B. F., Kudaravalli, S., Wen, X. & Pritchard, J. K. PLoS Biol 4, e72 (2006)). The presence of markers with possible indication of selective pressure has been inspected within pigmentation genes (Lao, O., de Gruijter, J. M., van Duijn, K., Navarro, A. & Kayser, M. Ann Hum Genet (2007)). By comparable methods, the dopa chrome tautomerase (DCT) was identified as a candidate gene for underlying skin pigmentation differences among human populations (Myles, S., Somel, M., Tang, K., Kelso, J. & Stoneking, M. Hum Genet 120, 613-21 (2007)). Furthermore, an association of polymorphism to skin colour variation within admixed populations and Europeans has been reported (Graf, J.,

Voisey, J., Hughes, I. & van Daal, A. Hum Mutat (2007), Graf, J., Hodgson, R. & van Daal, A. Hum Mutat 25, 278-84 (2005)).

A large part of the knowledge in the field of human pigmentation is focused on rare Mendelian syndromes of pigmentation anomalies like albinisms (Oetting, W. S., Fryer, J. P., Shriram, S. & King, R. A. Pigment Cell Res 16, 307-11 (2003)) and Hermansky Pudlack Syndromes (Wei, M. L. Pigment Cell Res 19, 19-42 (2006)). However, a limited number of genes have been confirmed to account for normal variation of pigmentation within ethnic groups. Thus, while variants within OCA2 explain in part normal variation patterns in eye colour and MC1R variants can be used for predicting probability of red hair colour, there is still a large fraction of eye colour and most of hair colour determinants that remain unaccounted for. In addition, a majority of the genetic variance in skin sensitivity to sun is still unexplained.

Knowledge of genetic variants that determine pigmentation in humans has implications for forensic testing. Genetic determinants for hair and eye colour, as well as skin pigmentation, can be utilized to aid in the identification of individuals, starting from even small quantities of genetic material. There is thus a need for an understanding of the genetic variants that determine human pigmentation patterns, for use in methods and kits for determining such characteristics, thus aiding in the identification of individuals based on their pigmentation appearance patterns.

Melanoma

Prevalence and Epidemiology. Cutaneous Melanoma (CM) was once a rare cancer but has over the past 40 years shown rapidly increasing incidence rates. In the U.S.A. and Canada, CM incidence has increased at a faster rate than any other cancer except bronchogenic carcinoma in women. Until recently incidence rates increased at 5-7% a year, doubling the population risk every 10-15 years.

The current worldwide incidence is in excess of 130,000 new cases diagnosed each year [Parkin, et al., (2001), Int J Cancer, 94, 153-6.]. The incidence is highest in developed countries, particularly where fair-skinned people live in sunny areas. The highest incidence rates occur in Australia and New Zealand with approximately 36 cases per 100,000 per year. The U.S.A. has the second highest worldwide incidence rates with about 11 cases per 100,000. In Northern Europe rates of approximately 9-12 per 100,000 are typically observed, with the highest rates in the Nordic countries. Currently in the U.S.A., CM is the sixth most commonly diagnosed cancer (excluding non-melanoma skin cancers). In the year 2008 it is estimated that 62,480 new cases of invasive CM will have been diagnosed in the U.S.A. and 8,420 people will have died from metastatic melanoma. A further 54,020 cases of in-situ CM are expected to be diagnosed during the year.

Deaths from CM have also been on the increase although at lower rates than incidence. However, the death rate from CM continues to rise faster than for most cancers, except non-Hodgkin's lymphoma, testicular cancer and lung cancer in women [Lens and Dawes, (2004), Br J Dermatol, 150, 179-85.]. When identified early, CM is highly treatable by surgical excision, with 5 year survival rates over 90%. However, malignant melanoma has an exceptional ability to metastasize to almost every organ system in the body. Once it has done so, the prognosis is very poor. Median survival for disseminated (stage IV) disease is 7½ months, with no improvements in this figure for the past 22 years. Clearly, early detection is of paramount importance in melanoma control.

CM shows environmental and endogenous host risk factors, the latter including genetic factors. These factors interact with each other in complex ways. The major environmental risk factor is

UV irradiation. Intense episodic exposures rather than total dose represent the major risk [Markovic, et al., (2007), Mayo Clin Proc, 82, 364-80].

It has long been recognized that pigmentation characteristics such as light or red hair, blue eyes, fair skin and a tendency to freckle predispose for CM, with relative risks typically 1.5-2.5. Numbers of nevi represent strong risk factors for CM. Relative risks as high as 46-fold have been reported for individuals with >50 nevi. Dysplastic or clinically atypical nevi are also important risk factors with odds ratios that can exceed 30-fold [Xu and Koo, (2006), Int J Dermatol, 45, 1275-83].

Genetic Testing for Melanoma. Relatives of melanoma patients are themselves at increased risk of melanoma, suggesting an inherited predisposition [Amundadottir, et al., (2004), PLoS Med, 1, e65. Epub 2004 Dec 28.]. A series of linkage based studies implicated CDKN2a on 9p21 as a major CM susceptibility gene [Bataille, (2003), Eur J Cancer, 39, 1341-7.]. CDK4 was identified as a pathway candidate shortly afterwards, however mutations have only been observed in a few families worldwide[Zuo, et al., (1996), Nat Genet, 12, 97-9.]. CDKN2a encodes the cyclin dependent kinase inhibitor p16 which inhibits CDK4 and CDK6, preventing G1-S cell cycle transit. An alternate transcript of CKDN2a produces p14ARF, encoding a cell cycle inhibitor that acts through the MDM2-p53 pathway. It is likely that CDKN2a mutant melanocytes are deficient in cell cycle control or the establishment of senescence, either as a developmental state or in response to DNA damage. Overall penetrance of CDKN2a mutations in familial CM cases is 67% by age 80. However penetrance is increased in areas of high melanoma prevalence [Bishop, et al., (2002), Natl Cancer Inst, 94, 894-903.].

Endogenous host risk factors for CM are in part under genetic control. It follows that a proportion of the genetic risk for CM resides in the genes that underpin variation in pigmentation and nevi. The Melanocortin 1 Receptor (MC1R) is a G-protein coupled receptor involved in promoting the switch from pheomelanin to eumelanin synthesis. Numerous, well characterized variants of the MC1R gene have been implicated in red haired, pale skinned and freckle prone phenotypes.

There is an unmet clinical need to identify individuals who are at increased risk of melanoma. Such individuals might be offered regular skin examinations to identify incipient tumours, and they might be counselled to avoid excessive UV exposure. Chemoprevention either using sunscreens or pharmaceutical agents [Bowden, (2004), Nat Rev Cancer, 4, 23-35.] might be employed. For individuals who have been diagnosed with melanoma, knowledge of the underlying genetic predisposition may be useful in determining appropriate treatments and evaluating risks of recurrence and new primary tumours.

Basal Cell Carcinoma and Squamous Cell Carcinoma

Prevalence and Epidemiology. Cutaneous basal cell carcinoma (BCC) is the most common cancer amongst whites and incidence rates show an increasing trend. The average lifetime risk for Caucasians to develop BCC is approximately 30% [Roewert-Huber, et al., (2007), Br J Dermatol, 157 Suppl 2, 47-51]. Although it is rarely invasive, BCC can cause considerable morbidity and 40-50% of patients will develop new primary lesions within 5 years[Lear, et al., (2005), Clin Exp Dermatol, 30, 49-55]. Indices of exposure to ultraviolet (UV) light are strongly associated with risk of BCC[Xu and Koo, (2006), Int J Dermatol, 45, 1275-83]. In particular, chronic sun exposure (rather than intense episodic sun exposures as in melanoma) appears to be the major risk factor [Roewert-Huber, et al., (2007), Br J Dermatol, 157 Suppl 2, 47-51]. Photochemotherapy for skin conditions such as psoriasis with psoralen and UV irradiation (PUVA) have been associated with increased risk of SCC and BCC. Immunosuppressive treatments increase the incidence of both SCC and BCC, with the incidence rate of BCC in transplant receipients being up to 100 times the population risk [Hartevelt, et al., (1990), Transplantation, 49, 506-9; Lindelof, et al., (2000), Br J Dermatol, 143, 513-9]. BCC's may be particularly aggressive in immunosuppressed individuals.

Genetic Testing for BCC and SCC. A positive family history is a risk factor for SCC and BCC [Hemminki, et al., (2003), Arch Dermatol, 139, 885-9; Vitasa, et al., (1990), Cancer, 65, 2811-7] suggesting an inherited component to the risk of disease. Several rare genetic conditions have been associated with increased risks of BCC and/or SCC, including Nevoid Basal Cell Syndrome (Gorlin's Syndrome), Xeroderma Pigmentosum (XP), and Bazex's Syndrome. XP is underpinned by mutations in a variety of XP complementation group genes. Gorlin's Syndrome results from mutations in the PTCH1 gene. In addition, variants in the CYP2D6 and GSTT1 genes have been associated with BCC [Wong, et al., (2003), Bmj, 327, 794-8]. Polymorphisms in numerous genes have been associated with SCC risk.

Fair pigmentation traits are known risk factors for BCC and SCC and are thought act, at least in part, through a reduced protection from UV irradiation. Therefore, risk variants for fair pigmentation may confer risk of BCC and SCC, although there are indications that such variants may have increased utility in BCC and SCC screening over and above what can be obtained from observing patients' pigmentation phenotypes.

There is an unmet clinical need to identify individuals who are at increased risk of BCC and SCC. Such individuals might be offered regular skin examinations to identify incipient tumours, and they might be counselled to avoid excessive UV exposure. Chemoprevention either using sunscreens or pharmaceutical agents [Bowden, (2004), Nat Rev Cancer, 4, 23-35.] might be employed. For individuals who have been diagnosed with BCC or SCC, knowledge of the underlying genetic predisposition may be useful in determining appropriate treatments and evaluating risks of recurrence and new primary tumours. Screening for susceptibility to BCC or SCC might be important in planning the clinical management of transplant recipients and other immunosuppressed individuals.

### SUMMARY OF THE INVENTION

The present invention discloses variants that contribute to human pigmentation patterns and risk of skin cancer phenotypes, including melanoma, basal cell carcinoma and squamous cell carcinoma. These variants can be utilized for the determination of the natural pigmentation patterns of a human individual, from a sample of genetic material, and for risk assessment of human skin cancers.

In a first aspect, the present invention relates to a method of inferring at least one pigmentation trait of a human individual, the method comprising determining the identity of at least one allele of at least one polymorphic marker in a nucleic acid sample from the individual, wherein the at least one marker is selected from the group of markers set forth in Table 10, and markers in linkage disequilibrium therewith, wherein the presence of the at least one allele is indicative of the at least one pigmentation trait of the individual. Information about the identity of at least one allele of at least one polymorphic marker can optionally also be obtained from a dataset that is derived from the individual. Thus, in certain embodiments, information about the identity of alleles of polymorphic markers can also be obtained from a genotype dataset. Inferring a pigmentation trait indicates that based on the genotype status of the at least one polymorphic marker, at least one particular pigmentation trait of the individual from which the sample originates can be inferred. In specific embodiments, inferring can be done to a predetermined level of confidence. Using genotype data from a group of individuals, prediction rules for predicting at least one pigmentation trait can be developed, as described in detail and exemplified herein. The predetermined level of confidence can be set forth as a percentage. For example, the pigmentation trait can be determined to a predetermined level of at least 90%, i.e. the particular individual has at least a 90% probability of having the particular pigmentation trait based on the genotype data for the at least one polymorphic marker that is assessed. The predetermined level can be any level that has been determined for the particular polymorphic marker, or combination of markers, employed, including 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, 15%, and 10% probability of the individual having the at least one polymorphic traits. Other whole-integer or fractional values spanning these values are also contemplated, and within the scope of the invention.

Another aspect of the invention relates to a method of inferring at least one pigmentation trait of a human individual, the method comprising the steps of:
(a) Determining the identity of at least one allele of at least one polymorphic marker in the MC1R gene that is associated with the at least one pigmentation trait;
(b) Determining the identity of at least one allele of at least one polymorphic marker in the OCA2 gene that is associated with the at least one pigmentation trait; and
(c) Determining the identity of at least one allele of at least one polymorphic marker selected from the markers set forth in Table 10C and 10D, and markers in linkage disequilibrium therewith;

wherein the presence or absence of the at least one allele in step (a), (b) and (c) is indicative of the at least one pigmentation trait of the individual

Another aspect of the invention relates to the use of genetic profiling for assessing the pigmentation pattern of a human individual, the genetic profiling comprising the steps of

(i) determining the identity of at least one allele of at least one polymorphic marker in a nucleic acid sample from the individual, or in a genotype dataset from the individual, wherein the at least one marker is selected from the group of markers set forth in Table 10, and markers in linkage disequilibrium therewith, to establish a genetic profile; and

(ii) calculating, to a predetermined level of confidence, the natural pigmentation pattern of the human individual, based on the genetic profile.

Another aspect of the invention relates to a procedure for determining the natural pigmentation pattern of a human individual, comprising:

(i) analyzing a nucleic acid from the human individual to assess at least one polymorphic marker selected from the markers set forth in Table 10, and markers in linkage disequilibrium therewith;

(ii) determining the status of a genetic indicator of a particular pigmentation trait in the individual from the measurement of the at least one marker;

wherein the status of the genetic indicator is a measure of the natural pigmentation pattern of the human individual.

Another aspect of the invention relates to the use of an oligonucleotide probe in the manufacture of a diagnostic reagent for assessing the natural pigmentation pattern of a human individual, wherein the probe comprises a fragment of the genome comprising at least one polymorphism selected from the polymorphisms set forth in Table 10, and polymorphisms in linkage disequilibrium therewith, wherein the fragment is 15-500 nucleotides in length.

In particular embodiments of the methods, uses and procedures of the invention, the at least one polymorphic marker is selected from the markers set forth in Table 10B -10D, and markers in linkage disequilibrium therewith. In other embodiments, the at least one polymorphic marker is selected from the markers set forth in Table 10C-10D, and markers in linkage disequilibrium therewith. In yet another embodiment, the at least one polymorphic marker is selected from the markers set forth in Table 10D, and markers in linkage disequilibrium therewith.

In certain embodiments, the invention relates to methods of determining the identity of at least one allele of at least one polymorphic marker set forth in Table 10B, 10C and/or 10D, and further comprising determining the identity of at least one allele of at least one polymorphic marker selected from the markers set forth in Table 10A. In certain other embodiments, the invention relates to methods of determining the identity of at least one allele of at least one polymorphic marker set forth in 10C and/or 10D, and further comprising determining the identity of at least one allele of at least one polymorphic marker selected from the markers set forth in Table 10A and/or at least one allele of at least one polymorphic marker selected from the markers set forth in Table 10B. Markers in linkage disequilibrium with these markers can also be used to practice the invention. Using a combination of at least one polymorphism as set forth in Tables 10C and 10D, and at least one polymorphism as set forth in Table 10A, optionally also including at least one polymorphism as set forth in Table 10B, the method of inferring at least one polymorphic trait can be practiced. Alternatively, using a combination of at least one polymorphism as set forth in Tables 10C and 10D, and at least one polymorphism as set forth in Table 10B, optionally also including at least one polymorphism as set forth in Table 10A, the method of inferring at least one polymorphic trait can be practiced.

One preferred embodiment of the invention comprises determining the identity of at least one allele of each of the polymorphic markers rs12896399, rs12821256, rs1540771, rs1393350, rs1042602, rs1667394, rs7495174, rs1805008, rs1805007, or markers in linkage disequilibrium therewith. The specific alleles identified comprises in one embodiment rs12896399 allele T, rs12821256 allele C, rs1540771 allele A, rs1393350 allele A, rs1042602 allele C, rs1667394 allele A, rs7495174 allele A, rs1805008 allele T and rs1805007 allele T. In one additional embodiment, the method further comprises determining the identity of at least one allele of at least one marker selected from the markers set forth in Table 10D, and markers in linkage disequilibrium therewith.

The pigmentation trait assessed in the methods, used, procedures and kits of the invention are in preferred embodiments selected from skin pigmentation, eye pigmentation and hair pigmentation. The pigmentation trait is in certain embodiments characterized by a particular colour of the hair, eye and/or skin of the individual. It is contemplated that other descriptive measures of the appearance of the pigmentation pattern may be employed, such as the shape, distribution, and/or spectral properties characteristic of the pigmentation trait of interest, and such measures are also useful for practicing the invention.

In one embodiment, the hair colour is selected from blond, brown, black and red hair. Other embodiments can include other hair colours, such as black ink, dark, domino, ebony, jet black, midnight, onyx, raven, raveonette, sable, chestnut, chocolate, cinnamon, dark, mahogany, dirty blond, dishwater blond, flaxen, fair, golden, honey, platinum blond, sandy blond, champagne blond, strawberry blonde, yellow, strawberry blonde, auburn, chestnut, cinnamon, fiery, ginger, russet, scarlet, titian, blond-brown, red-brown, reddish brown, brown-black and dark brown.

In one embodiment, the pigmentation trait of the invention is hair pigmentation and the at least one polymorphic marker is selected from rs896978, rs3750965, rs2305498, rs1011176, rs4842602, rs995030, rs1022034, rs3782181, rs12821256, rs4904864, rs4904868, rs2402130, rs7495174, rs7183877, rs8039195, rs1667394 and rs1540771, and markers in linkage disequilibrium therewith. In another embodiment, the pigmentation trait is hair colour and the at least one polymorphic marker is selected from rs896978, rs3750965, rs2305498, rs1011176, rs4842602, rs995030, rs1022034, rs3782181, rs12821256, rs4904864, rs4904868, rs2402130 and rs1540771, and markers in linkage disequilibrium therewith.

In certain embodiments of the invention, the pigmentation trait is eye pigmentation. The eye pigmentation can be described by a descriptive colour. In one such embodiment, the pigmentation pattern of the eye is described by at least one colour selected from blue, steel blue, brown, grey, steel grey, olive, blue-green, hazel, amber and violet. Other colours or combination of colours can also be used to describe the characteristic pigmentation pattern of the eye, and are also within scope of the invention. In one embodiment, the pigmentation trait inferred by the methods and kits of the invention is eye colour, and the at least one polymorphic marker is selected from rs1022901, rs10809808, rs11206611, rs12441723, rs1393350, rs1408799, rs1448488, rs1498519, rs1584407, rs1667394, rs16950979, rs16950987, rs1907001, rs2240204, rs2402130, rs2594935, rs2703952, rs2871875, rs4453582, rs4778220, rs4904864, rs4904868, rs630446, rs6497238, rs7165740, rs7170869, rs7183877, rs728405, rs7495174, rs7680366, rs7684457, rs8016079, rs8028689, rs8039195, rs927869, and markers in linkage disequilibrium therewith. In another embodiment, the at least one polymorphic marker is selected from rs4453582, rs7684457, rs7680366, rs11206611, rs1393350, rs8016079, rs4904864, rs4904868, rs2402130, rs1408799, rs630446, rs11206611, rs1393350, rs1022901, rs10809808 and rs927869, and markers in linkage disequilibrium therewith.

The present invention also relates to skin pigmentation. A useful descriptive measure of the appearance of skin is its colour. Thus, in one embodiment, the skin pigmentation trait is skin colour. In another embodiment, the skin pigmentation trait is characterized by the absence or presence of freckles. The descriptive measure of the presence or absence of freckles can optionally also include description of skin colour. Another measure of skin pigmentation trait that is useful and is within the scope of the invention is skin sensitivity to sun. One embodiment therefore refers to skin pigmentation as described by the skin sensitivity to the sun. A useful definition of skin sensitivity to the sun is provided by the Fitzpatrick skin-type score (Fitzpatrick, T. B., Arch Dermatol 124, 869-71 (1988)). Any combination of descriptive measures of skin pigmentation is also possible, and may be useful in certain embodiments of the invention. This includes, but is not limited to, the combination of skin colour and the presence and/or absence of freckles, skin sensitivity to the sun and the presence and/or absence of freckles, skin colour and skin sensitivity to the sun. Any particular descriptive skin colour or combination of skin colours can be employed in such embodiments. Skin colour is typically described by a continuum from white to black. In one embodiment, skin colour is described by at least one colour selected from white, yellow, brown and black. Other skin colour are also useful, including but not limited to, yellow-brown, yellowish brown, light brown, dark brown, and brown-black. Another descriptive measure of skin colour includes fair, dark and very dark, which may also be employed in certain embodiments.

In one embodiment of the invention, the pigmentation trait is skin pigmentation, and the at least one polymorphic marker is selected from rs4911379, rs2284378, rs4911414, rs2225837, rs6120650, rs2281695, rs6059909, rs2378199, rs2378249, rs6060034, rs6060043, rs619865, rs11242867, rs9378805, rs9328192, rs9405681, rs4959270, rs1540771, rs1393350, rs1042602, rs1050975, rs872071, rs7757906, rs950286, rs9328192, rs9405675 and rs950039, and markers in linkage disequilibrium therewith. In another embodiment, the at least one polymorphic marker is selected from rs1042602, rs1050975, rs9503644, rs1393350, rs1540771, rs2225837, rs2281695, rs2284378, rs2378199, rs2378249, rs4911379, rs4911414, rs4959270, rs6059909, rs6060034, rs6060043, rs6120650, rs619865, rs7757906, rs872071, rs9328192, rs9378805, rs9405675, rs9405681, rs950039 and rs950286, and markers in linkage disequilibrium therewith. In one embodiment, the pigmentation is skin pigmentation characterised by the presence of allele G at marker rs1015362 and allele T at marker rs4911414. Correspondingly, in one embodiment determination of the presence of allele G at marker rs1015362 and allele T at marker rs4911414 is performed, and wherein of both of these alleles is indicative of the skin pigmentation trait in the individual. In one embodiment, skin sensitivity to sun is determined by the Fitzpatrick skin-type score.

The methods, uses and procedures of the invention can in certain embodiments further comprise assessing frequency of at least one haplotype for at least two polymorphic markers, wherein the presence of the haplotype is indicative of the at least one pigmentation trait in the individual. Any combination of markers can be useful in such embodiment. In one embodiment, the haplotype represents a linkage disequilibrium (LD) block in the human genome, and such haplotypes are sometimes referred to as block haplotypes, which may be useful in some embodiments.

Variants associated with skin pigmentation are in one embodiment also useful for diagnosing a risk for, or a susceptibility to, cancer, in particular skin cancer. Thus, one embodiment of the invention relates to a method of diagnosing a susceptibility to skin cancer in a human individual, the method comprising determining the presence or absence of at least one allele of at least one polymorphic marker in a nucleic acid sample obtained from the individual, wherein the presence of the at least one allele is indicative of a susceptibility to skin cancer. In one embodiment, the skin cancer is melanoma. In a preferred embodiment, the at least one polymorphic marker is rs6060043 or rs1393350, and markers in linkage disequilibrium therewith. In another preferred embodiment, the at least one polymorphic marker is marker rs1015362 and marker rs4911414, and wherein determination of a haplotype comprising allele G at marker rs1015362 and allele T at marker rs4911414 is indicative of increased risk of melanoma cancer. In another embodiment the at least one polymorphic marker is selected from rs2424994, rs6060009, rs6060017, rs6060025, rs3787223, rs910871, rs3787220, rs6060030, rs1884432, rs6088594, rs6060034, rs6058115, rs6060047, rs7271289, rs2425003, rs17092148, rs11546155, rs17122844 and rs7265992.

Certain aspects of the invention relate to methods of determining susceptibility to skin cancer phenotypes. Certain embodiments relate to skin cancers selected from melanoma, basal cell carcinoma and squamous cell carcinoma. Preferred embodiments relate to skin cancers selected from melanoma and basal cell carcinoma.

In one such aspect, the invention pertains to a method of determining a susceptibility to a skin cancer in a human individual, the method comprising (a) determining the presence or absence of at least one allele of at least one polymorphic marker in a nucleic acid sample obtained from the individual, or in a genotype dataset from the individual, wherein the at least one polymorphic marker is associated with at least one gene selected from the ASIP gene, the TYR gene and the TYRP1 gene, and (b) determine a susceptibility to the skin cancer based on the presence of the at least one allele of the at least one polymorphic marker.

Another aspect provides a method of determining a susceptibility to a skin cancer in a human individual, comprising (a) obtaining sequence data about a human individual, wherein the data includes identification of at least one allele of at least one polymorphic marker associated with at least one gene selected from the ASIP gene, the TYR gene and the TYRP1 gene, wherein different alleles of the at least one polymorphic marker are associated with different susceptibilities to the skin cancer in humans, and (b) determining a susceptibility to the skin cancer from the sequence data of the individual.

In some embodiments, the at least one marker is selected from the group consisting of marker, rs1015362, rs4911414, rs1126809, rs1408799, rs6060043, and rs1393350, and markers in linkage disequilibrium therewith. In one preferred embodiment, the at least one marker is rs1126809. In another preferred embodiment, the at least one marker is rs4911414.

In some embodiments, the markers in linkage disequilibrium with rs1126809, which is associated with the TYR gene, are selected from the group consisting of rs3913310, rs17184781, rs7120151, rs7126679, rs11018434, rs17791976, rs7931721, rs11018440, rs11018441, rs10830204, rs11018449, rs477424, rs7929744, rs7127487, rs10830206, rs4121738, rs11018463, rs11018464, rs3921012, rs7944714, rs10765186, rs9665831, rs1942497, rs2156123, rs7930256, rs4420272, rs7480884, rs12363323, rs1942486, rs10830216, rs17792911, rs4121729, rs10830219, rs10830228, rs10830231, rs7127661, rs10830236, rs949537, rs5021654, rs12270717, rs621313, rs7129973, rs11018525, rs17793678, rs594647, rs10765196, rs10765197, rs7123654, rs11018528, rs12791412, rs12789914, rs7107143, rs574028, rs2000553, rs11018541, rs10765198, rs7358418, rs10765200, rs10765201, rs4396293, rs2186640, rs10501698, rs10830250, rs7924589, rs4121401, rs10741305, rs591260, rs1847134, rs1393350, rs1126809, rs1827430, rs3900053, rs1847142, rs501301, rs4121403, rs10830253, rs7951935, rs1502259, rs1847140, rs1806319, rs4106039, rs4106040, rs10830256, rs3793973 and rs1847137, which are the markers set forth in Table 25 herein.

In certain embodiments, markers in linkage disequilibrium with rs1408799, which is associated with the TYRP1 gene are selected from the group consisting of rs791675, rs1325131, rs10756375, rs1590487, rs791691, rs791696, rs791697, rs702132, rs702133, rs702134, rs10960708, rs10809797, rs10429629, rs10960710, rs1022901, rs962298, rs6474717, rs1325112, rs1325113, rs4428755, rs10756380, rs10756384, rs13283146, rs1408790, rs1408791, rs10960716, rs713596, rs1325115, rs1325116, rs1408792, rs10809806, rs13288558, rs2025556, rs1325117, rs6474718, rs13283649, rs1325118, rs10738286, rs7466934, rs10960721, rs7036899, rs10756386, rs10960723, rs4612469, rs977888, rs10809808, rs10756387, rs10960730, rs10809809, rs10125059, rs10756388, rs10960731, rs10960732, rs7026116, rs10124166, rs7047297, rs13301970, rs10960735, rs1325122, rs6474720, rs6474721, rs10960738, rs13283345, rs10809811, rs1408794, rs1408795, rs13294940, rs1325124, rs996697, rs2382359, rs995263, rs1325125, rs10435754, rs4741242, rs2209275, rs7022317, rs1121541, rs10809818, rs1325127, rs10960748, rs9298679, rs9298680, rs7863161, rs1041105, rs10960749, rs1408799, rs1408800, rs13294134, rs16929340, rs13299830, rs10960751, rs10960752, rs10960753, rs16929342, rs16929345, rs16929346, rs13296454, rs13297008, rs10116013, rs10809826, rs7847593, rs13293905, rs2762460, rs2762461, rs2762462, rs2762463, rs2224863, rs2733830, rs2733831, rs2733832, rs2733833, rs2209277, rs2733834, rs683, rs2762464, rs910, rs1063380, rs9298681, rs10960758, rs10960759, rs12379024, rs13295868, rs7019226, rs11789751, rs10491744, rs10960760, rs2382361, rs1409626, rs1409630, rs13288475, rs13288636, rs13288681, rs1326798, rs7871257, rs12379260, rs13284453, rs13284898, rs7048117, rs10756400, rs970944, rs970945, rs970946, rs970947, rs10960774, rs10756402, rs10756403, rs10738290, rs13300005, rs10756406, rs7019486, rs927868, rs7019981, rs927869, rs4741245, rs7023927, rs7035500, rs13302551, rs1543587, rs1074789, rs2181816, rs10125771, rs10960779, rs1326789, rs7025842, rs7025953, rs7025771, rs7025914, rs10491743, rs1326790, rs1326791, rs1326792, rs7030485, rs10960781, rs12115198, rs10960783, rs1041176, rs10119113, rs1326795, rs2209273, rs7855624, rs10491742, and rs3750502, which are the markers set forth in Table 26 herein.

Certain embodiments relate to the identification of at least two polymorphic markers. In certain embodiments, haplotypes are determined comprising at least two polymorphic markers. In one preferred embodiment, the haplotype is the haplotype comprising rs1015362 allele A and rs4911414 allele T, which is also called AH haplotype herein. The at least one polymorphic marker associated with the ASIP gene may thus be a marker in linkage disequilibrium with the haplotype comprising rs1015362 allele A and rs4911414 allele T. In some embodiments, the markers in linkage disequilibrium with the AH haplotype are selected from the group consisting of rs1885120, rs17401449, rs291671, rs291695, rs293721, rs721970, rs910873, rs17305573, rs4911442, rs1204552, rs293709, rs6058091, rs1884431, rs6142199, rs2068474, rs2378199, rs2378249, rs2425003, rs4302281, rs4564863, rs4911430, rs6059928, rs6059937, rs6059961, rs6059969, rs6087607, rs2144956, rs2295443, rs2889849, rs6058089, rs6059916, rs932542, rs17421899, rs1884432, rs7265992, rs17092148, rs3787220, rs3787223, rs6058115, rs6060009, rs6060017, rs6060030, rs6060034, rs6060043, rs6060047, rs6088594, rs7271289, rs910871, rs6088316, rs17396317, rs2425067, rs6058339, rs6060612, rs2378412, rs293738, rs1205339, rs2281695, rs4911154, rs6088515, rs7269526, rs17305657, rs1122174, rs6060025, rs6059908, rs4911523, rs4911315, rs619865, rs6059931, rs11546155, rs221981, rs17122844, rs7272741, rs2425020, rs2424941, rs761930, rs221984, rs2378078, rs2424944, rs633784, rs666210, rs7361656, rs2424948, rs2424994, rs221985, rs17092378, rs2050652, rs6058192, rs6059662, and rs7274811, which are the markers set forth in Table 14 herein.

In preferred embodiments, at-risk alleles predictive of increased susceptibility to the at least one skin cancer are identified. In certain embodiments, the the at least one allele or haplotype comprises at least one allele selected from the group consisting of rs1015362 allele G, rs4911414 allele T, rs1126809 allele A, rs1408799 allele C, rs6060043 allele C, and rs1393350 allele A.

Sequence data obtained in certain aspects of the invention relate to the identification of particular marker alleles. For single nucleotide polymorphisms, such sequence data may thus represent a single nucleotide of a nucleic acid, or a single amino acid at the protein level. Obtaining sequence data therefore comprises obtaining sequence data about at least the nucleotide position(s) representing the polymorphic variation. If the polymorphism represents a single nucleotide, then sequence information about the particular nucleotide positions is minimally obtained. For longer polymorphisms stretching across two or more nucleotides, additional sequence information is obtained to be able to identify the particular marker allele. Additional sequence information may optionally also be obtained.

In certain embodiments, obtaining nucleic acid sequence data comprises obtaining a genotype dataset from the human individual and analyzing sequence of the at least one polymorphic marker in the dataset. In certain embodiments, analyzing sequence of at least one polymorphic marker comprises determining the presence or absence of at least one allele of the at least one: polymorphic marker. The sequence data can be nucleic acid sequence or alternatively it can be amino acid sequence data. The sequence data can in certain embodiments be obtained from a preexisting record. In some embodiments, determining a susceptibility comprises comparing the sequence data to a database containing correlation data between the at least one polymorphic marker and susceptibility to the skin cancer. In certain embodiments, the database comprises at least one measure of susceptibility to the skin cancer for the at least one polymorphic marker. The database can in certain embodiments comprise a look-up table comprising at least one measure of susceptibility to the skin cancer for the at least one polymorphic marker.

The invention further relates to a method of screening a candidate marker for assessing susceptibility to at least one skin cancer selected from the group consisting of melanoma, basal cell carcinoma and squamous cell carcinoma, comprising analyzing the frequency of at least one allele of a polymorphic marker associated with at least one of the ASIP gene, the TYR gene and the TYRP1 gene, in a population of human individuals diagnosed with the skin cancer, wherein a significant difference in frequency of the at least one allele in the population of human individuals diagnosed with the skin cancer as compared to the frequency of the at least one allele in a control population of human individuals is indicative of the marker as a susceptibility marker for the skin cancer.

Further, the invention relates to a method of identification of a marker for use in assessing susceptibility to at least one skin cancer selected from melanoma, basal cell carcinoma and squamous cell carcinoma, the method comprising:
a. identifying at least one polymorphic marker in linkage disequilibrium with at least one of the ASIP gene, the TYR gene and the TYRP1 gene;
b. determining the genotype status of a sample of individuals diagnosed with, or having a susceptibility to, the skin cancer; and
c. determining the genotype status of a sample of control individuals;

wherein a significant difference in frequency of at least one allele in at least one polymorphism in individuals diagnosed with the skin cancer, as compared with the frequency of the at least one allele in the control sample is indicative of the at least one polymorphism being useful for assessing susceptibility to the skin cancer. In certain embodiments, an increase in frequency of, the at least one allele in the at least one polymorphism in individuals diagnosed with, or having a susceptibility to, the skin cancer, as compared with the frequency of the at least one allele in the control sample is indicative of the at least one polymorphism being useful for assessing increased susceptibility to the skin cancer.

The invention also provides genotyping methods of the markers shown herein to be associated with pigmentation and skin cancer. One such aspect relates to a method of genotyping a nucleic acid sample obtained from a human individual comprising determining the identity of at least one allele of at least one polymorphic marker in a nucleic acid sample from the individual, wherein the at least one marker is associated with at least one of the ASIP gene, the TYR gene and the TYRP1 gene, and wherein determination of the presence of the at least one allele in the sample is indicative of a susceptibility to at least one skin cancer selected from melanoma, basal cell carcinoma and squamous cell carcinoma in the individual.

In certain embodiments of the invention, linkage disequilibrium between markers is defined as r²>0.1 (r² greater than 0.1). In another embodiment, linkage disequilibrium is defined as r²>0.2 (r² greater than 0.2). Other embodiments can include other definitions of linkage disequilibrium, such as r²>0.25, r²>0.3, r²>0.35, r²>0.4, r²>0.45, r²>0.5, r²>0.55, r²>0.6, r²>0.65, r²>0.7, r²>0.75, r²>0.8, r²>0.85, r²>0.9, r²>0.95, r²>0.96, r²>0.97, r²>0.87, or r²>0.99. Linkage disequilibrium can in certain embodiments also be defined as |D′|>0.2, or as |D′|>0.3, |D′|>0.4, |D′|>0.5, |D′|>0.6, |D′|>0.7, |D′|>0.8, |D′|>0.9, |D′|>0.95, |D′|>0.98 or |D′|>0.99. In certain embodiments, linkage disequilibrium is defined as fulfilling two criteria of r² and |D′|, such as r²>0.2 and |D′|>0.8. Other combinations of values for r²and |D′| are also possible and within scope of the present invention, including but not limited to the values for these parameters set forth in the above.

The present invention also relates to kits. Thus, in one embodiment, the invention relates to a kit for assessing the natural pigmentation pattern of a human individual, the kit comprising reagents for selectively detecting at least one allele of at least one polymorphic marker in a genomic DNA sample from the individual, wherein the polymorphic marker is selected from the group consisting of the polymorphic markers listed in Table 10, and markers in linkage disequilibrium therewith, and wherein the presence of the at least one allele is indicative of the natural pigmentation pattern of the individual. (specific embodiments to 10B, 10C, 10D). In one embodiment, the invention relates to a kit for assessing a susceptibility to skin cancer, (e.g., melanoma) in an individual. In one such embodiment, the polymorphic marker is selected from rs6060043 and markers in linkage disequilibrium therewith. In one embodiment, the genomic.

DNA comprising the at least one polymorphic marker is characterized by the sequence set forth in SEQ ID NO: 1-134). In another embodiment, the reagents comprise at least one contiguous oligonucleotide that hybridizes to a fragment of the genome of the individual comprising the at least one polymorphic marker, a buffer and a detectable label. In yet another embodiment, the reagents comprise at least one pair of oligonucleotides that hybridize to opposite strands of a genomic segment obtained from the subject, wherein each oligonucleotide primer pair is designed to selectively amplify a fragment of the genome of the individual that includes one polymorphic marker, and wherein the fragment is at least 30 base pairs in size. In a preferred embodiment, the at least one oligonucleotide is completely complementary to the genome of the individual. The oligonucleotide is in one embodiment about 18 to about 50 nucleotides in length. In another embodiment, the oligonucleotide is 20-30 nucleotides in length.

The kit may also be useful for assessing susceptibility to a skin cancer phenotype. Thus, certain aspects provide a kit for assessing susceptibility to at least one skin cancer selected from melanoma, basal cell carcinoma and squamous cell carcinoma in a human individual, the kit comprising reagents for selectively detecting at least one allele of at least one polymorphic marker in the genome of the human individual, wherein the polymorphic marker is a marker associated with at least one of the ASIP gene, the TYR gene and the TYRP1 gene and a collection of data comprising correlation data between the at least one polymorphic marker and susceptibility to the skin cancer in humans.

In one embodiment of the kits of the invention, reagents for detection of each at least one polymorphic marker include:
(i) A first oligonucleotide probe that is from 5-100 nucleotides in length and specifically hybridizes (under stringent conditions) to a first segment of a nucleic acid that comprises at least one polymorphic site selected from the list of polymorphic markers set forth in Tables 10 and 11,
wherein the oligonucleotide probe comprises a detectable label at its 3′ terminus and a quenching moiety at its 5′ terminus;
(ii) an enhancer oligonucleotide that is from 5-100 nucleotides in length and that is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid; wherein a single base gap exists between the first segment and the second segment, such that when the oligonucleotide probe and the enhancer oligonucleotide probe are both hybridized to the nucleic acid, a single base gap exists between the oligonucleotides;
(iii) treating the nucleic acid with an endonuclease that will cleave the detectable label from the 3′ terminus of the detection probe to release free detectable label when the detection probe is hybridized to the nucleic acid; and
(iv) measuring free detectable label, wherein the presence of the free detectable label indicates that the detection probe specifically hybridizes to the first segment of the nucleic acid, and indicates the sequence of the polymorphic site as the complement of the detection probe.

In one such embodiment, the nucleotide sequence of the nucleic acid that comprises at least one polymorphic site is given by SEQ ID NO: 1-138.

In certain alternative embodiments, the first oligonucleotide probe specifically hybridizes (under stringent conditions) to a first segment of a nucleic acid with sequence as set forth in any one of SEQ ID NO:139-483 herein.

In another embodiment, the kit further comprises at least one oligonucleotide pair for amplifying a genomic fragment comprising at least one polymorphism as listed in Table 10, Table 11, Table 14, Table 25 or Table 26, the genomic fragment being from 40-500 nucleotides in length. Other embodiments include those that relate to markers shown herein to be associated with skin cancer. Certain such embodiments relate to the markers disclosed herein to be associated with the TYR, TYRP1 and ASIP genes.

In certain embodiments of the kits of the invention, instructions for calculating, to a predetermined level of confidence, the natural pigmentation pattern of the human individual, based on the genotype status of the at least one polymorphic marker detected using the reagents in the kit, are provided. Such instructions can refer to tables relating specific combinations of marker alleles at one or more polymorphic site to the probability of a specific pigmentation trait, or to a combination of pigmentation traits. As shown herein, certain polymorphic markers are associated with multiple pigmentation traits, and assessment of one such marker can therefore provide information about more than one pigmentation trait. The instructions can also relate to combinations of a plurality of markers, for which the level of confidence of various pigmentation traits, as defined herein, are provided to a predetermined level of confidence, based on the presence or absence of at least one allele of the plurality of markers assessed.

In certain embodiments of the invention, the characteristic hair colour is selected from blond, brown, black and red hair colour. As hair colour is usually a continuous trait, i.e. with a continuum of hair shades and/or colour, categorization of hair colour can be performed by a variety of methods. The invention therefore also pertains to other shades of hair colour, including, but not limited to, black ink, dark, domino, ebony, jet black, midnight, onyx, raven, raveonette, sable, chestnut, chocolate, cinnamon, dark, mahogany, dirty blond, dishwater blond, flaxen, fair, golden, honey, platinum blond, sandy blond, champagne blond, strawberry blonde, yellow, strawberry blonde, auburn, chestnut, cinnamon, fiery, ginger, russet, scarlet, titian, blond-brown, red-brown, reddish brown, brown-black and dark brown. The hair colour can be self reported. The hair colour can also be determined by objective mesures, such as by visual inspection of an independent observer, either from an image, such as a colour photograph or by visual inspection of the individual in question.

Eye colour is determined primarily by the amount and type of pigments present in the eye's iris.

In humans, variations in eye colour are attributed to varying ratios of eumelanin produced by melanocytes in the iris. Three main elements within the iris contribute to its colour: the melanin content of the iris pigment epithelium, the melanin content within the iris stroma, and the cellular density of the iris stroma. In eyes of all colours, the iris pigment epithelium contains the black pigment, eumelanin. Colour variations among different irises are typically attributed to the melanin content within the iris stroma. The density of cells within the stroma affects how much light is absorbed by the underlying pigment epithelium. Human eye colour exists on a continuum from the darkest shades of brown to the lightest shades of blue (Sturm, R. A. & Frudakis, T. N., Trends Genetics 8:327-332 (2004)), although the most common used categorical labels of eye colour are probably blue, brown and green eye colour. There are 3 true colours in the eyes that determine the outward appearance; brown, yellow, and gray. How much of each colour you have determines the appearance of the eye colour. The colour your eyes turn depends on how much of these colours are present. For example, green eyes have a lot of yellow and some brown, making them appear green. Blue eyes have a little yellow and little to no brown, making them appear blue. Gray eyes appear gray because they have a little yellow and no brown in them. Brown eyes appear brown because most of the eye contains the brown colour. Brown is the most common, blue is second, and green is rarest. Based on a need for a standardized classification system that was simple, yet detailed enough for research purposes, a scale based on the predominant iris colour has been developed. On this scale, which describes the appearance of the eye in terms of its colour, the colours brown, light brown, green, gray, and blue are specified. Other descriptive terms for eye colours that are commonly used are steel blue, steel grey, olive, blue-green, hazel, amber and violet. Amber coloured eyes are of a solid colour and have a strong yellowish/golden and russet/coppery tint. Amber eyes are also nicknamed “cat eyes”. In humans, yellow specks or patches are thought to be due to the pigment lipofuscin, also known as lipochrome. Hazel eyes are believed to be due to a combination of a Rayleigh scattering and a moderate amount of melanin in the iris' anterior border layer. A number of studies using three-point scales have assigned “hazel” to be the medium-colour between light brown and dark green. This can sometimes produce a multicoloured iris, i.e., an eye that is light brown near the pupil and charcoal or amber/dark green on the outer part of the iris when it is open to the elements of the sun/shined in the sunlight. Hazel is mostly found in the regions of Southern and Eastern Europe, Britain, and the Middle East. The eye colour “hazel” is also sometimes considered to be synonymous with light brown and other times with dark green, or even yellowish brown or as a lighter shade of brown. In North America, “hazel” is often used to describe eyes that appear to change colour, ranging from light brown to green and even blue, depending on current lighting in the environment. The variants of the present invention have been shown to be correlated with human eye colour. The variants are therefore useful for inferring human eye colour from a nucleic acid sample. In the present context therefore, the term “eye colour” refers to eye colour as defined by any of these criteria, or by other methods or descriptive labels used to define eye colour. In the present context, eye colour can either be self-reported, or it is determined by an independent observer, by visual inspection or from an image, including colour photographs.

Skin colour is determined by the amount and type of the pigment melanin in the skin. On average, women have slightly lighter skin than men. Dark skin protects against those skin cancers that are caused by mutations in skin cells induced by ultraviolet light. Light-skinned persons have about a tenfold greater risk of dying from skin cancer under equal sun conditions. Furthermore, dark skin prevents UV-A radiation from destroying the essential B vitamin folate. Folate is needed for the synthesis of DNA in dividing cells and too low levels of folate in pregnant women are associated with birth defects. While dark skin protects vitamin B, it can lead to a vitamin D deficiency. The advantage of light skin is that it does not block sunlight as effectively, leading to increased production of vitamin D₃, necessary for calcium absorption and bone growth. The lighter skin of women may result from the higher calcium needs of women during pregnancy and lactation. One theory on the origin of dark skin speculates that haired ancestors of humans, like modern great apes, had light skin under their hair. Once the hair was lost, they, evolved dark skin, needed to prevent low folate levels since they lived in sun-rich Africa. When humans migrated to less sun-intensive regions in the north, low vitamin D₃ levels became a problem and light skin colour re-emerged. Albinism is a condition characterized by the absence, of melanin, resulting in very light skin and hair.

Human skin tone or skin colour is highly variable, ranging from very light or almost white to black. The lightest skin tone is typically found in northern Europe, with the darkest skin tone in sub-Saharan Africa and in native Australians.

The present invention relates to skin pigmentation traits that are a result of the pigmentation pattern in the skin. The descriptive Fitzpatrick sun sensitivity scale is useful since it categorizes skin tone or skin colour according to the sensitivity of the sun to the ultraviolet radiation of the sun. The variants of the present invention that are correlated to skin pigmentation are also useful for inferring the skin tone or skin colour of an individual, and such use is also within the scope of the invention. Variations in frequency of the associated variants in populations dominated by different skin colours (see, e.g., Table 9) illustrates this utility.

Freckles represent an additional phenotypic trait of skin pigmentation. Freckles are small coloured spots of melanin on the exposed skin or membrane of people with complexions fair enough for them to be visible. It is commonly believed that freckles have a genetic basis, and variants in the melanocortin-1 receptor MC1R gene variant have been described, that explain in part the heritability of freckles (Valverde, P., et al. Nat Genet 11, 328-30 (1995); Rees, J. L. Am J Hum Genet 75, 739-51 (2004)). Freckles can also be triggered by long exposure to sunlight, such as sun tanning. When the sun's rays penetrate the skin, they activate melanocytes which can cause freckles to become darker and more numerous, although the distribution of melanin is not the same. Fair hair such as blonde, or more commonly red hair, are usually common with the genetic factor of freckles, but none so much as fair or pale skin. There is thus a relationship between fair or pale skin, sun sensitivity and freckles. Freckles are predominantly found on the face, although they may appear on any skin exposed to the sun. People with a predisposition to freckles may be especially susceptible to sunburn and skin cancer. The present invention relates to polymorphic markers that are associated to freckles, and are thus useful for predicting whether an individual is likely to experience freckles naturally, or as a result of exposure to sun (i.e., tanning). While the present invention relates to self-report of the presence or absence of freckles, other descriptive categorization of the freckle trait is also useful for practising the invention, and therefore within its scope. For example, freckles may be assessed in a quantitative manner, such as by simple counting of freckles on a given bodypart (e.g., face), or by limitation to specific body parts. Description of freckles can be practised as a self-report, or by an objective examination by a third party (e.g., a doctor, or other health professional), either by direct visual inspection or by determination from an image, such as a colour photograph.

The invention also provides computer-readable media. Such media in general have computer executable instructions for determining susceptibility to a skin cancer selected from melanoma, basal cell carcinoma and squamous cell carcinoma, or alternativelyl instructions for predicing the pigmentation pattern of a human individual, the computer readable medium comprising:

data indicative of at least one polymorphic marker;

a routine stored on the computer readable medium and adapted to be executed by a processor to determine risk of developing the at least skin cancer or at least one pigmentation trait for the at least one polymorphic marker.

The markers can be selected from any one or a combination of the markers shown herein to be associated with human pigmentation and skin cancer, respectively, as further described herein.

The invention also provides apparatus for determining genetic indicators. Such indicators can for example be genetic indicators for a skin cancer as described herein. The indicators may also be indicators of a particular pigmentation pattern of a human individual. The apparaturs preferably comprises a processor, and a computer readable memory having computer executable instructions adapted to be executed on the processor to analyze marker and/or haplotype information for at least one human individual with respect to at least one polymorphic marker or a haplotype that is associated with risk of the skin cancer or is associated with the at least one pigmentation trait, and generate an output based on the marker or haplotype information, wherein the output comprises a measure of susceptibility of the at least one marker or haplotype as a genetic indicator of the skin cancer for the human individual, or alternatively the output comprises a prediction measure for the at least one pigmentation trait.

In certain embodiments, the computer readable memory further comprises data indicative of the frequency of at least one allele of at least one polymorphic marker or the at least one haplotype in a plurality of individuals diagnosed with, or presenting symptoms associated with, the at least one skin cancer, or alternatively individuals individuals with a particular pigmentation trait, and data indicative of the frequency of at the least one allele of at least one polymorphic marker or the at least one haplotype in a plurality of reference individuals, and wherein a measure of susceptibility of the skin cancer or a prediction of the pigmentation trait is based on a comparison of the at least one marker and/or haplotype status for the human individual to the data indicative of the frequency of the at least one marker and/or haplotype information for the plurality of individuals diagnosed with the skin cancer or individuals with the particular pigmentation trait.

In certain embodiments, the computer readable memory further comprises data indicative of the risk of developing at least one skin cancer associated with at least one allele of the at least one polymorphic marker or the at least one haplotype, or a data predictive of a particular pigmentation trait for the at least one marker or haplotype, and wherein a measure of susceptibility or prediction for the human individual is based on a comparison of the at least one marker and/or haplotype status for the human individual to the risk or probability associated with the at least one allele of the at least one polymorphic marker or the at least one haplotype.

In certain embodiments, the computer readable memory further comprises data indicative of the frequency of at least one allele of at least one polymorphic marker or at least one haplotype in a plurality of individuals diagnosed with, or presenting symptoms associated with, the at least one skin cancer, or alternatively in individuals with a particular pigmentation trait, and data indicatie of the frequency of at the least one allele of at least one polymorphic marker or at least one haplotype in a plurality of reference individuals, and wherein risk of developing the at least one skin cancer, or prediction of the particular pigmentation trait, is based on a comparison of the frequency of the at least one allele or haplotype in individuals diagnosed with, or presenting symptoms associated with, the skin cancer, or individuals with the particular pigmentation trait, and reference individuals.

It should be understood that all combinations of features described herein are contemplated, even if the combination of feature is not specifically found in the same sentence or paragraph herein. This includes in particular the use of all markers disclosed herein, alone or in combination, for analysis individually or in haplotypes, in all aspects of the invention as described herein.

### BRIEF DESCRIPTION OF THE DRAWINGS

FIG. 1 shows a schematic representation of how different genetic variants associate to pigmentation. For eye and hair colour, each cell shows how frequent the genetic variant is for each phenotype relative to the population frequency of the variant. For sun sensitivity and freckles, each cell shows how frequent the variant is compared to people that are not sensitive to sun or have not had freckles, respectively. The odds ratio (OR) scale is used to compare frequencies. For simplicity, only cells corresponding to characteristics with reasonably significant association (P<0.001) are shaded, the degree of shading correlating with the significance of association. Cells corresponding to highly significant (P<1×10⁻⁸) results from the six genome-wide scans are marked with a (*). Cells with decreased frequence of the particular allele are marked with an (L). For simplicity, only one variant is shown for each of the MC1R and OCA2 regions, as the other variant has different association profiles for both regions.

FIG. 2 shows an overview of accuracy of eye (FIG. 2A) and hair (FIG. 2B) pigmentation prediction based on genotype status of markers rs12896399, rs12821256, rs1540771, rs1393350, rs1042602, rs1667394, rs7495174, rs1805008, and rs1805007. Bars indicate, from left to right, blue eyes, green eyes and brown eyes, respectively, (FIG. 2A); and red hair, blond hair, dark blond or light brown hair, and brown or black hair, respectively (FIG. 2B). The prediction rules were created from the Icelandic discovery sample and then applied to the Icelandic and Dutch replication samples. Only those individuals who were genotyped for all necessary markers, or good surrogates of these markers, were used. Histograms show the distribution of pigmentation within each sample and within groups of individuals with similar predicted pigmentation. The percentage cutoffs indicated represent the degree to which each pigmentation treat can be predicted, i.e. the percentage is a measure of the predetermined level to which the particular trait can be inferred. For example, in FIG. 2A, the genotype status can be used to predict brown hair in individuals to at least 50% accuracy. In the Icelandic discovery cohort, 259 individuals fulfill the criteria, and indeed over 60% of them have brown hair. In the, Dutch replication cohort, 210 individuals fulfill the criteria, and again over 60% of those have brown hair, validating the prediction.

FIG. 3-FIG. 8 show allelic association of SNP's with main skin and eye pigmentation characteristics. The small horizontal dots show all the genotyped SNP's indicating the coverage of each genomic region. The large dots correspond to the SNP's tested for association. The recombination hot spots are shown by the vertical strips. Genes are represented at the bottom by lines, with the exons as thin vertical bars and with an arrow indicating transcriptional direction. Due to the high density of genes, the graphical description of the genes was simplified in FIG. 7, where their location is indicated by thin lines.

FIG. 9 shows the genomic region of chromosome 20q11.22 that includes marker rs6060043 that is significantly associated with human pigmentation and melanoma cancer. Genes in the region are indicated by horizontal lines, and where vertical bars indicate exons, and arrowheads the transcriptional direction of each gene. Recombination hotspots are indicated by thick black bar, and linkage disequilibrium in the HapMap CEU population by the pairwise LD pattern plot at the bottom (the darker the shade, the greater the LD).

FIG. 10 shows association results to freckling and burning in a 4 Mb segment on chromosome 20. X indicates single SNP P-values of association. Solid lines indicate P-values for all two marker haplotype in the region with P<10⁻¹⁵. Genes in the regions are indicated by their abbreviated name and a solid line below each name. The most significant association is observed for haplotypes in a region that contains the ASIP gene.

FIG. 11 shows estimates of Odds Ratio (OR) for haplotypes at ASIP (a) and at TPCN2 (b). At ASIP, the previously reported mutation 8818A is compared to the variant (AH) in individuals who burn and freckle and those who tan and do not freckle. Chromosomes not carrying AH are denoted by notAH. At TPCN2 the two missense mutations G734E and M484L are compared to the wild type haplotype and to each other. Frequencies in the two pigmentation groups are displayed in brackets. Estimated ORs and P-values, from the pair-wise comparison of the haplotype at the end of arrow versus haplotype at the beginning of the arrow adjusted for all other haplotypes, are displayed beside each arrow.

FIG. 12 shows an exemplary computer environment on which the methods and apparatus as described and claimed herein can be implemented.

### DETAILED DESCRIPTION OF THE INVENTION

Definitions

The following terms shall, in the present context, have the meaning as indicated.

A “polymorphic marker”, sometimes referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including single nucleotide polymorphisms (SNPs), microsatellites, insertions, deletions, duplications and translocations. Polymorphic markers can be of any measurable frequency in the population. For mapping of disease genes, polymorphic markers with population frequency higher than 5-10% are in general most useful. However, polymorphic markers may also have lower population frequencies, such as 1-5% frequency, or even lower frequency, in particular copy number variations (CNVs). The term shall, in the present context, be taken to include polymorphic markers with any population frequency.

An “allele” refers to the nucleotide sequence of a given locus (position) on a chromosome. A polymorphic marker allele thus refers to the composition (i.e., sequence) of the marker on a chromosome. Genomic DNA from an individual contains two alleles for any given polymorphic marker, representative of each copy of the marker on each chromosome. Sequence codes for nucleotides used herein are: A=1, C=2, G=3, T=4. For microsatellite alleles, the CEPH sample (Centre d'Etudes du Polymorphisme Humain, genomics repository, CEPH sample 1347-02) is used as a reference, the shorter allele of each microsatellite in this sample is set as 0 and all other alleles in other samples are numbered in relation to this reference. Thus, e.g., allele is 1 by longer than the shorter allele in the CEPH sample, allele 2 is 2 by longer than the shorter allele in the CEPH sample, allele 3 is 3 by longer than the lower allele in the CEPH sample, etc., and allele −1 is 1 by shorter than the shorter allele in the CEPH sample, allele −2 is 2 by shorter than the shorter allele in the CEPH sample, etc.

Sequence conucleotide ambiguity as described herein is as proposed by IUPAC-IUB. These codes are compatible with the codes used by the EMBL, GenBank, and PIR databases.

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C, G or T |
| D | A, G or T |
| H | A, C or T |
| V | A, C or G |
| N | A, C, G or T (Any base) |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP”, as defined herein, refers to a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA.

A “marker” or a “polymorphic marker”, as defined herein, is a variant. Alleles that differ from the reference are referred to as “variant” alleles.

A “fragment” of a nucleotide or a protein, as described herein, comprises all or a part of the nucleotide or the protein.

An “animal”, as described herein, refers to any domestic animal (e.g., cats, dogs, etc.), agricultural animal (e.g., cows, horses, sheep, chicken, etc.), or test species (e.g., rabbit, mouse, rat, etc.), and also includes humans.

A “microsatellite” is a polymorphic marker that has multiple small repeats of bases that are 2-8 nucleotides in length (such as CA repeats) at a particular site, in which the number of repeat lengths varies in the general population.

An “indel” is a common form of polymorphism comprising a small insertion or deletion that is typically only a few nucleotides long.

A “haplotype,” as described herein, refers to a segment of genomic DNA within one strand of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles. Haplotypes are described herein in the context of the marker name and the allele of the marker in that haplotype, e.g., “G rs1015362 T rs4911414”, or alternatively “rs1015362 G rs4911414 T” refers to the G allele of marker rs1015362 and the T allele of marker rs4911414 being in the haplotype, and is equivalent to “rs1015362 allele G rs4911414 allele T”. Furthermore, allelic codes in haplotypes are as for individual markers, i.e. 1=A, 2=C, 3=G and 4=T.

The term “susceptibility”, as described herein, refers to the proneness of an individual towards the development of a certain state (e.g., a certain trait, phenotype or disease), or towards being less able to resist a particular state than the average individual. The term encompasses both increased susceptibility and decreased susceptibility. Thus, particular alleles at polymorphic markers and/or haplotypes of the invention as described herein may be characteristic of increased susceptibility (i.e., increased risk) of a skin cancer, as characterized by a relative risk (RR) or odds ratio (OR) of greater than one for the particular allele or haplotype. Alternatively, the markers and/or haplotypes of the invention are characteristic of decreased susceptibility (i.e., decreased risk) of the skin cancer, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or the can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a computer using a commercially available or custom-made interface. Exemplary compute-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or acess of stored information. Such transmission can be electrical, or by other available methods, such as IR links, wireless connections, etc.

A “nucleic acid sample” is a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including as a blood sample, sample of amniotic fluid, sample of cerebrospinal fluid, or tissue sample from skin, muscle, buccal or conjunctival mucosa (buccal swab), placenta, gastrointestinal tract or other organs.

The term “polypeptide”, as described herein, refers to a polymer of amino acids, and not to a specific length; thus, peptides, oligopeptides and proteins are included within the definition of a polypeptide

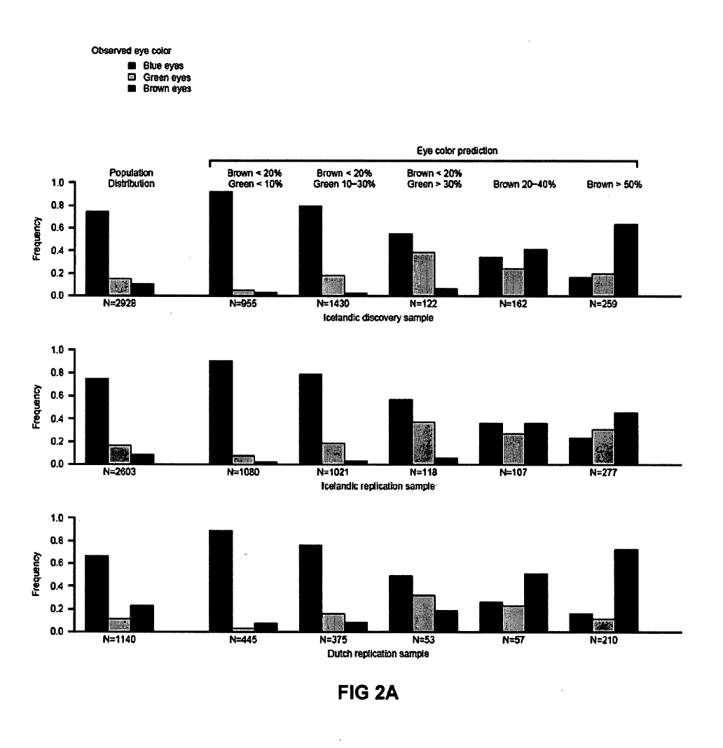

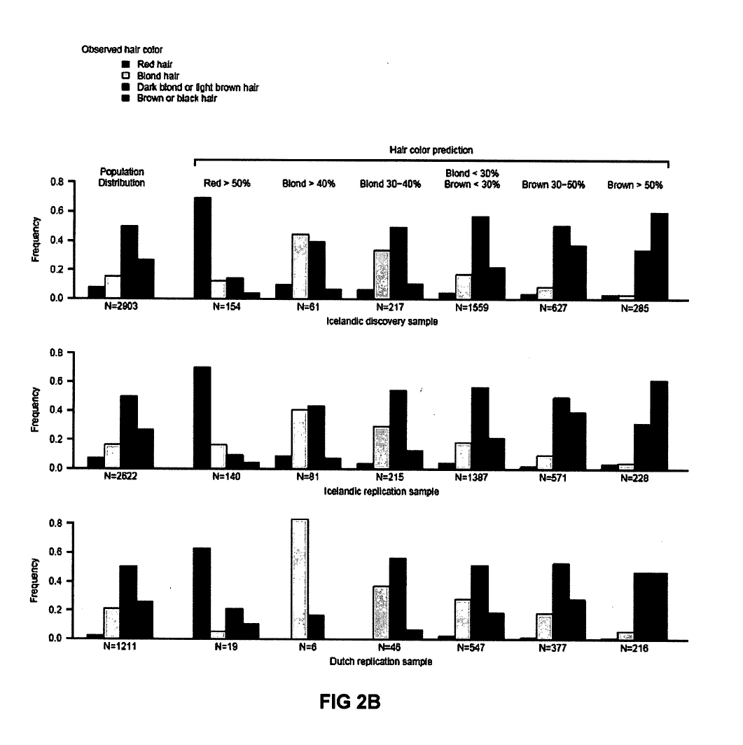

The term “infer” or “inferring”, as described herein, refers to methods of determining the likelihood of a particular trait, in particular a pigmentation trait of an individual. The likelihood can be determined by comparing genotype status, either at a single polymorpic site (i.e., for one polymorphic marker), or for a plurality of polymorphic markers, either within a single locus or from several loci in the genome. By comparing observed genotypes to the relative risk, or the odds ratio, conferred by each particular marker that is assessed, or haplotypes comprising at least two such markers, the particular pigmentation trait, or several pigmentation traits, can be inferred by methods such as those described in detail herein (e.g., as illustrated in FIG. 2). In certain embodiments, a pigmentation trait of an individual is inferred, i.e. determined, with a certain level of confidence. The level of confidence depends on the degree to which the particular polymorphic marker(s) that have been assessed relate to the particular trait being inferred, as described in detail herein.

The term “Fitzpatrick skin-type score”, as described herein, refers to self-assessed sensitivity of the skin to ultraviolet radiation (UVR) from the sun (Fitzpatrick, T. B., Arch Dermatol 124, 869-71 (1988)), where the lowest score (I) represents very fair skin that is very sensitive to UVR and the highest score (IV) represents dark skin that tans rather than burns in reaction to UVR exposure. In certain applications, individuals scoring I or II are classified as being sensitive to sun and individuals scoring III or IV on the Fitzpatrick skin-type score are classified as not being sensitive to sun.

The term “natural pigmentation pattern”, as described herein, refers to the eye, hair and/or skin pigmentation pattern of a human individual in its natural state, i.e. in the absence of any changes in the appearance of the individual or other modifications to the natural pigmentation. For example, natural hair pigmentation pattern refers to the natural hair colour of an individual, in the absence of changes or alterations in colour produced by colour dying. The natural eye pigmentation pattern of an individual refers to the pigmentation of the eye, as determined by its appearance, in the absence of modifications to its appearance, for example by use of coloured lenses. The natural skin pigmentation pattern of an individual refers to the natural skin pigmentation pattern in the absence of any cosmetic changes to the skin, i.e. in the absence of any cosmetic agents that alter its appearance (e.g., colour), or other artificial measures used to alter the appearance of an individual. Skin pigmentation pattern of an individual that is affected or altered (e.g., through appearance of freckles, or by burning or tanning) by natural sun radiation is considered natural skin pigmentation, as described herein.

The term “genomic fragment”, as described herein, refers to a continuous segment of human genomic DNA, i.e. a segment that contains each nucleotide within the given segment, as defined (e.g., by public genomic assemblies, e.g., NCBI Build 34, NCBI Build 35, NCBI Build 36, or other public genomic assemblies; or as defined by the nucleotide sequence of SEQ ID NO: 1-138).

The term “skin cancer”, as described herein, refers to any cancer affecting the skin of humans, including cancer that develops in the epidermis. The term includes Cutaneous Melanoma (CM), also called melanoma cancer, melanoma or malignant melanoma, basal cell carcinoma (BCC), and squamous cell carcinoma (SCC), and also dermatofibrosarcoma protuberans, Merkel cell carcinoma and Kaposi's sarcoma.

The term “ASIP”, as described herein , refers to the the Agouti Signaling Protein. The gene encoding the ASIP protein, also called ASIP herein, is located on human chromosome 20q11.22. The term “TYR”, as described herein, refers to the Tyrosinase protein. The gene encoding the TYR protein, also called TYR herein, is located chromosome 11814.3. The term, “TYRP1”, as described herein, refers to Tyrosinase-Related Protein 1. The gene encoding the TYRP1 protein, also called TYRP1, is located on human chromosome 9p23.

Genetic Association to Human Pigmentation Traits

The present inventors have found that certain polymorphic markers and haplotypes are associated with human pigmentation traits, e.g., natural hair colour, natural eye colour, skin sensitivity to sun assessed by Fitzpatrick score and presence of freckles. A number of single nucleotide polymorphisms (SNPs), and haplotypes comprising SNPs were found to be significantly associated with pigmentation traits. In particular, SNPs associated with pigmentation were found to cluster in distinct genomic locations on chromosomes 1, 4, 6, 9, 11, 12, 14, 15, 16, 18 and 20, as indicated in Table A. Representative results of analysis for specific pigmentation traits are provided by Examples 1-3 herein. Furthermore, as shown herein, the polymorphisms indicated in Table A may be used alone, or in combination, to estimate the risk of a particular pigmentation trait, or infer a particular pigmentation trait from genotype data for at least one of the SNP markers shown in Table A.

By way of example, the T allele of the polymorphic SNP marker rs12896399 can be used to assess the probability that a particular individual has blond as compared with brown hair (see, for example, Table 3). The C allele of marker rs12821256 can be used for the same purpose, as can the A allele of marker rs1540771, the A allele of marker rs1393350, the A allele of marker rs1667394, or the T allele of marker rs1805008. All of these markers are therefore useful for inferring blond as compared with brown hair of an individual, and represent one application of the present invention in forensic testing. Using a combination of markers provides additional power in such forensic testing, as described further herein.

A second example is illustrated by the association of markers to sun sensitivity. Markers that are associated to skin sun sensitivity are indicated in Table 4 herein. For example, the presence of the T allele of marker rs12896399, the A allele of marker rs1540771, the A allele of marker rs1393350, the A allele of marker rs1667394, the T allele of marker rs1805008 and the T allele of marker rs1805007 can all be used to estimate whether an individual is likely to have fair skin that burns easily when exposed to sunlight, as compared with dark skin that tans easily.

Results for a large number of other variants the present inventors have found to be associated with particular pigmentation traits are shown in Tables 2-5 and 10 herein. All the variants significantly associated with pigmentation traits can be utilized in methods for inferring at least one pigmentation trait, by determining the identity of at least one allele of at least one polymorphic marker in a nucleic acid sample from the individual, wherein the at least one marker is selected from the group of markers set forth in Tables 2-5 and Table 10, wherein the presence of the at least one allele is indicative of the at least one pigmentation trait of the individual

Furthermore, as described further herein, multiple signals detected within distinct genomic locations are likely to be due to linkage disequilibrium (LD) between the SNP markers in question in the region. As discussed in more detail in the following, the consequence of LD is that for each particular variant (polymorphic marker) found to be associated with a trait, a number of other polymorphic markers can also be used to detect the association. Markers that are in LD with the markers in Table A are indicated in Table 11 herein. The markers listed in Table 11 therefore represent alternative embodiments of the invention, as described in further detail herein.

Follow-up analyses within the region on chromosome 20 revealed that the underlying association appears to be due to a 2-marker haplotype (G rs1015362 T rs4911414; also called AH herein), since association to other single markers is not significant when corrected for AH (see Example 4 herein). Furthermore, additional variants on chromosome 11813.2 were identified as associating with hair colour, in a region that overlaps with the TPCN2 gene.

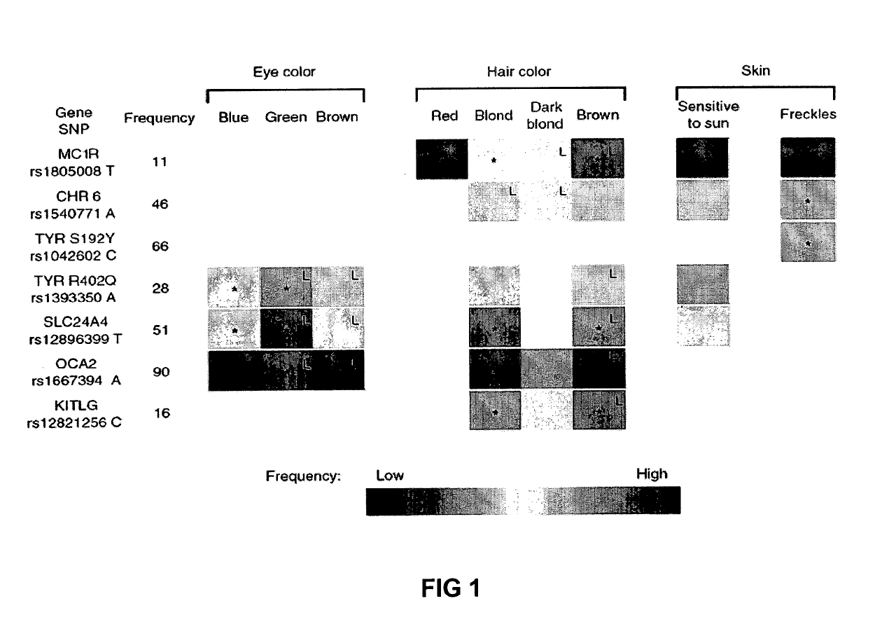

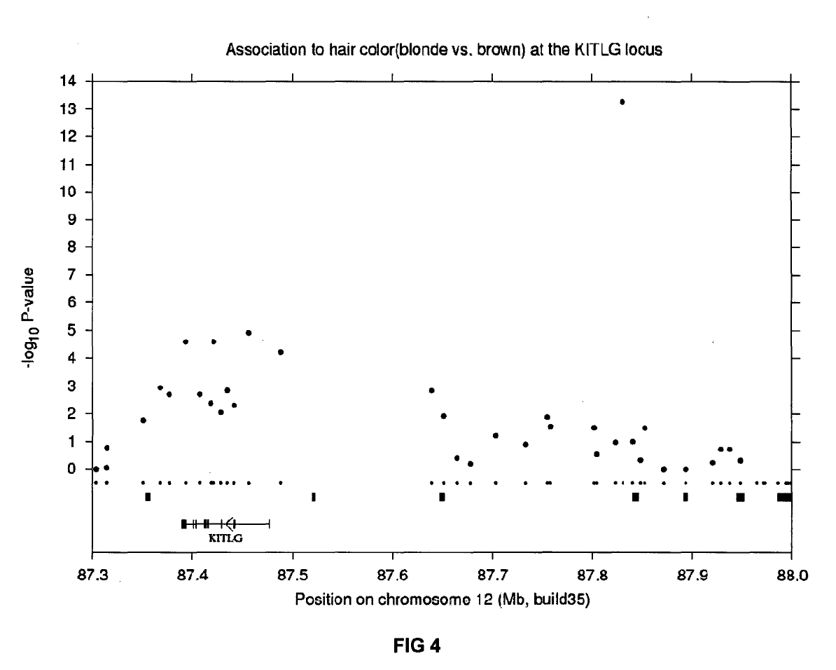

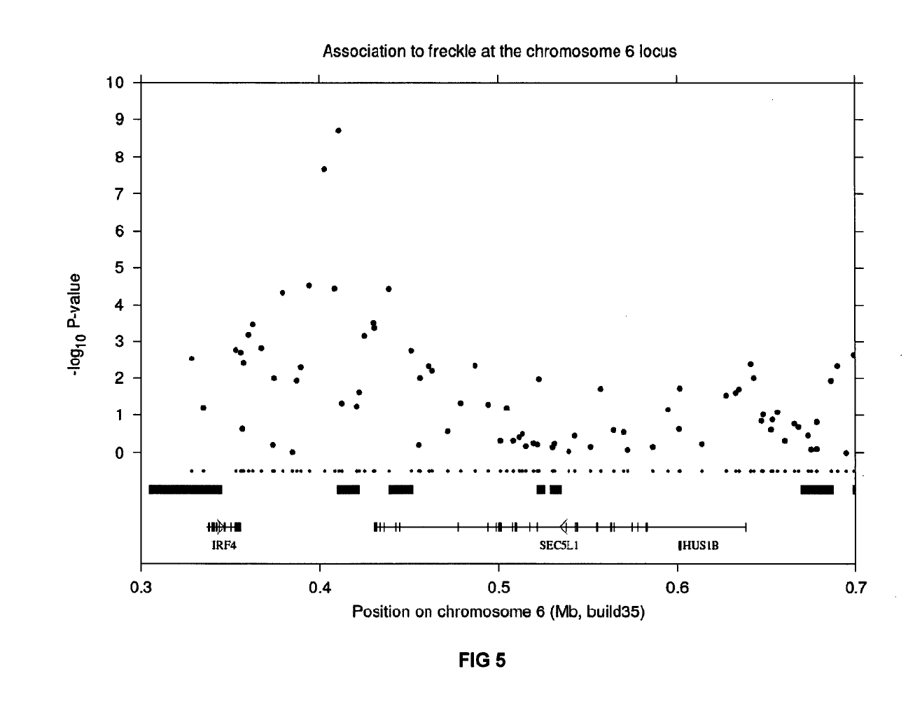

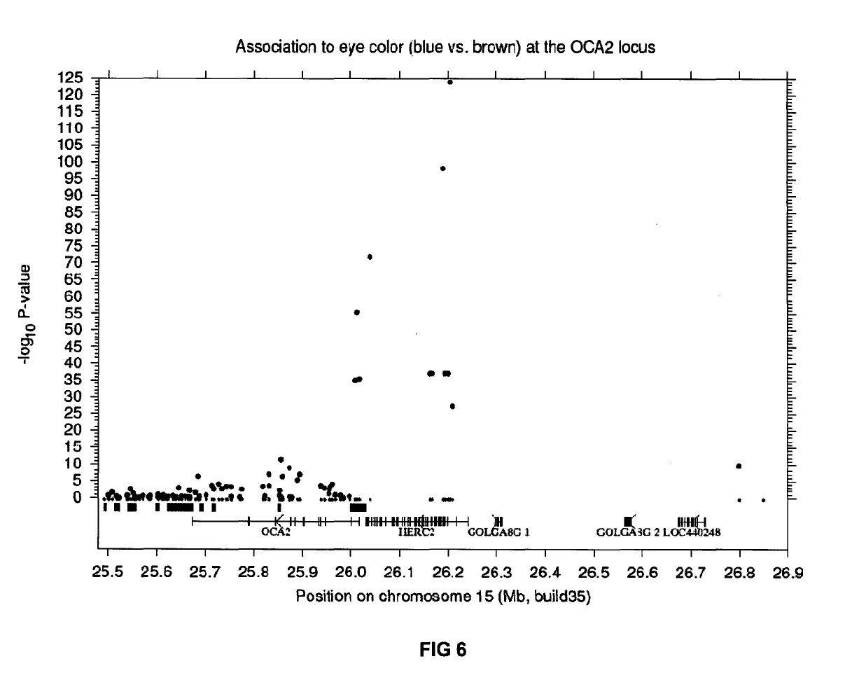

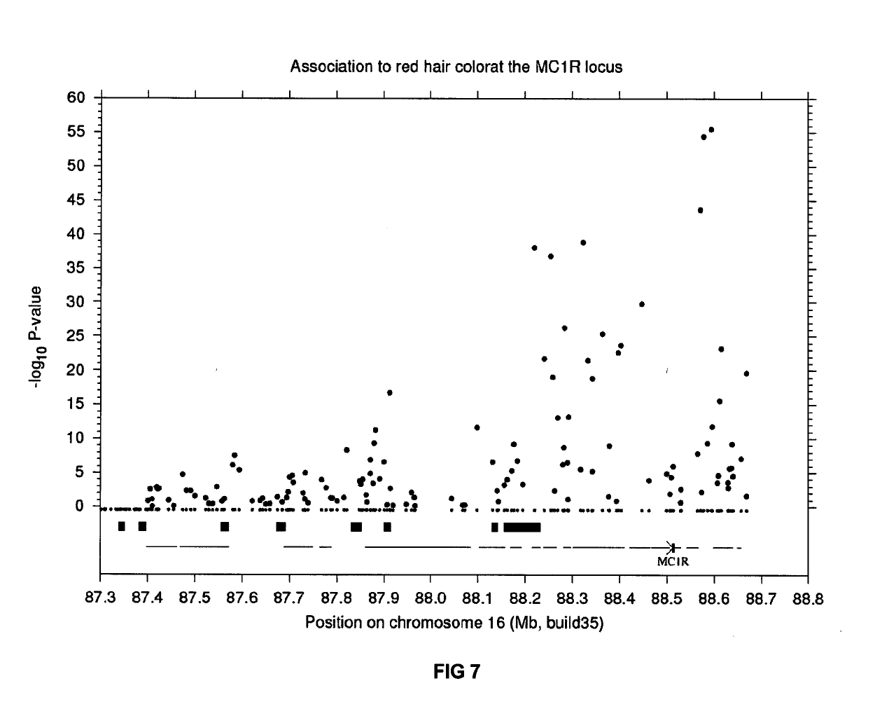

Examples 1-4 herein and corresponding data presented in Tables 1-19 and FIGS. 1-11 provide additional exemplification of the variants found to be associated with human pigmentation traits.

TABLE A
Representative Single Nucleotide Polymorphisms (SNPs)
found to be associated with at least one pigmentation trait.

| SNP | Chr | Position | SEQ |
|---|---|---|---|
| rs11206611 | chr1 | 55679165 | 9 |
| rs630446 | chr1 | 55662008 | 93 |
| rs7680366 | chr4 | 101929217 | 108 |
| rs7684457 | chr4 | 101882168 | 109 |
| rs1050975 | chr6 | 353012 | 6 |
| rs11242867* | chr6 | 360406 | 10 |
| rs9503644* | chr6 | 360406 | 10 |
| rs1540771 | chr6 | 411033 | 21 |
| rs4959270 | chr6 | 402748 | 87 |
| rs7757906 | chr6 | 357741 | 110 |
| rs872071 | chr6 | 356064 | 119 |
| rs9328192 | chr6 | 379364 | 124 |
| rs9378805 | chr6 | 362727 | 125 |
| rs9405675 | chr6 | 389600 | 126 |
| rs9405681 | chr6 | 394358 | 127 |
| rs950039 | chr6 | 438976 | 128 |
| rs950286 | chr6 | 374457 | 129 |
| rs1022901 | chr9 | 12578259 | 3 |
| rs10809808 | chr9 | 12614463 | 7 |
| rs1408799 | chr9 | 12662097 | 17 |
| rs927869 | chr9 | 12738962 | 123 |
| rs1011176 | chr11 | 68690473 | 1 |
| rs1042602 | chr11 | 88551344 | 4 |
| rs1393350 | chr11 | 88650694 | 16 |
| rs2305498 | chr11 | 68623490 | 43 |
| rs3750965 | chr11 | 68596736 | 59 |
| rs896978 | chr11 | 68585505 | 122 |
| rs1022034 | chr12 | 87421211 | 2 |
| rs12821256 | chr12 | 87830803 | 15 |
| rs3782181 | chr12 | 87456029 | 62 |
| rs4842602 | chr12 | 87235053 | 82 |
| rs995030 | chr12 | 87393139 | 134 |
| rs2402130 | chr14 | 91870956 | 49 |
| rs4904864 | chr14 | 91834272 | 83 |
| rs4904868 | chr14 | 91850754 | 84 |
| rs8016079 | chr14 | 91828198 | 111 |
| rs12441723 | chr15 | 27120318 | 13 |
| rs1448488 | chr15 | 25890452 | 18 |
| rs1498519 | chr15 | 25685246 | 20 |
| rs1584407 | chr15 | 25830854 | 23 |
| rs1667394 | chr15 | 26203777 | 25 |
| rs16950979 | chr15 | 26194101 | 26 |
| rs16950987 | chr15 | 26199823 | 27 |
| rs1907001 | chr15 | 27053851 | 31 |
| rs2240204 | chr15 | 26167627 | 37 |
| rs2594935 | chr15 | 25858633 | 52 |
| rs2703952 | chr15 | 25855576 | 53 |
| rs2871875 | chr15 | 25938449 | 54 |
| rs4778220 | chr15 | 25894733 | 74 |
| rs6497238 | chr15 | 25727373 | 94 |
| rs7165740 | chr15 | 27057792 | 96 |
| rs7170869 | chr15 | 25962343 | 97 |
| rs7183877 | chr15 | 26039328 | 98 |
| rs728405 | chr15 | 25873448 | 104 |
| rs7495174 | chr15 | 26017833 | 105 |
| rs8028689 | chr15 | 26162483 | 112 |
| rs8039195 | chr15 | 26189679 | 113 |
| rs1048149 | chr16 | 88638451 | 5 |
| rs11076747 | chr16 | 87584526 | 8 |
| rs11648785 | chr16 | 88612062 | 11 |
| rs11861084 | chr16 | 88403211 | 12 |
| rs12599126 | chr16 | 87733984 | 14 |
| rs1466540 | chr16 | 87871978 | 19 |
| rs154659 | chr16 | 88194838 | 22 |
| rs164741 | chr16 | 88219799 | 24 |
| rs16966142 | chr16 | 88378534 | 28 |
| rs1800286 | chr16 | 88397262 | 29 |
| rs1800359 | chr16 | 88332762 | 30 |
| rs1946482 | chr16 | 88289911 | 32 |
| rs2011877 | chr16 | 88342319 | 33 |
| rs2078478 | chr16 | 88657637 | 34 |
| rs2239359 | chr16 | 88376981 | 36 |
| rs2241032 | chr16 | 88637020 | 38 |
| rs2241039 | chr16 | 88615938 | 39 |
| rs2270460 | chr16 | 88499917 | 40 |
| rs2306633 | chr16 | 87882779 | 44 |
| rs2353028 | chr16 | 87880179 | 45 |
| rs2353033 | chr16 | 87913062 | 46 |
| rs258322 | chr16 | 88283404 | 50 |
| rs258324 | chr16 | 88281756 | 51 |
| rs2965946 | chr16 | 88044113 | 55 |
| rs3096304 | chr16 | 87901208 | 56 |
| rs3212346 | chr16 | 88509859 | 57 |
| rs352935 | chr16 | 88176081 | 58 |
| rs3751688 | chr16 | 88161940 | 60 |
| rs3751700 | chr16 | 88279695 | 61 |
| rs3785181 | chr16 | 88632834 | 63 |
| rs3803688 | chr16 | 88462387 | 64 |
| rs382745 | chr16 | 88131087 | 65 |
| rs4238833 | chr16 | 88578190 | 66 |
| rs4347628 | chr16 | 88098136 | 67 |
| rs4408545 | chr16 | 88571529 | 68 |
| rs455527 | chr16 | 88171502 | 70 |
| rs459920 | chr16 | 88258328 | 71 |
| rs460879 | chr16 | 88240390 | 72 |
| rs464349 | chr16 | 88183752 | 73 |
| rs4782497 | chr16 | 87546780 | 75 |
| rs4782509 | chr16 | 87354279 | 76 |
| rs4785612 | chr16 | 88640608 | 77 |
| rs4785648 | chr16 | 87855978 | 78 |
| rs4785755 | chr16 | 88565329 | 79 |
| rs4785763 | chr16 | 88594437 | 80 |
| rs4785766 | chr16 | 88629885 | 81 |
| rs6500437 | chr16 | 88317399 | 95 |
| rs7188458 | chr16 | 88253985 | 99 |
| rs7195066 | chr16 | 88363824 | 100 |
| rs7196459 | chr16 | 88668978 | 101 |
| rs7201721 | chr16 | 88586247 | 102 |
| rs7204478 | chr16 | 88322986 | 103 |
| rs7498845 | chr16 | 87594028 | 106 |
| rs7498985 | chr16 | 88630618 | 107 |
| rs8045560 | chr16 | 88506995 | 114 |
| rs8058895 | chr16 | 88342308 | 115 |
| rs8059973 | chr16 | 88607035 | 116 |
| rs8060934 | chr16 | 88447526 | 117 |
| rs8062328 | chr16 | 87343542 | 118 |
| rs885479 | chr16 | 88513655 | 120 |
| rs889574 | chr16 | 87914309 | 121 |
| rs9921361 | chr16 | 87821940 | 130 |
| rs9932354 | chr16 | 87580066 | 131 |
| rs9936215 | chr16 | 88609161 | 132 |
| rs9936896 | chr16 | 88596560 | 133 |
| rs4453582 | chr18 | 34735189 | 69 |
| rs2225837 | chr20 | 32469295 | 35 |
| rs2281695 | chr20 | 32592825 | 41 |
| rs2284378 | chr20 | 32051756 | 42 |
| rs2378199 | chr20 | 32650141 | 47 |
| rs2378249 | chr20 | 32681751 | 48 |
| rs4911379 | chr20 | 31998966 | 85 |
| rs4911414 | chr20 | 32193105 | 86 |
| rs6059909 | chr20 | 32603352 | 88 |
| rs6060034 | chr20 | 32815525 | 89 |
| rs6060043 | chr20 | 32828245 | 90 |
| rs6120650 | chr20 | 32503634 | 91 |
| rs619865 | chr20 | 33331111 | 92 |
| rs35264875 | chr11 | 68602975 | 135 |
| rs1015362 | chr20 | 32202273 | 136 |
| rs1126809 | chr11 | 88657609 | 137 |
| rs3829241 | chr11 | 68611939 | 138 |

*Marker rs11242867 is the same as rs9503644

Implications for Human Disease

Certain human diseases are correlated with the appearance or presence of certain pigmentation, traits. Variants associated with such pigmentation traits are therefore also possible disease-associated variants. If the pigmentation trait only occurs as a manifestation of the particular disease state, then the variants associated with the trait are by default also associated with the disease. However, certain pigmentation traits or pigmentation patterns are also known to increase the risk of developing certain diseases. Variants associated with such pigmentation traits are in those cases potential disease-associated variants, which can be tested in individuals with the particular disease. The variants in question may contribute to the appearance of the diseaese independent of the pigmentation trait, and the association effect is in that case observed through the associated pigmentation trait; alternatively, the variants are associated with the pigmentation trait but do not manifest their effect in individuals with the disease in the absence of the pigmentation trait. In such cases, the variants are associated with the pigmentation trait in the absence of the associated disease state. Alternatively, the observed risk in individuals with the disease can be lower than observed for the pigmentation trait, corresponding to the prevalence of the disease state in individuals with the particular pigmentation trait. In such a case, the variant contributes to the pigmentation trait, but does not provide additional risk of the disease state.

It is therefore contemplated that the variants of the inventions may be associated with at least one disease state associated with at least one of pigmentation traits described herein. The inventors contemplate that the variants of the invention (e.g., the polymorphic markers set forth in Table 10, or markers in linkage disequilibrium therewith) may be associated with pigmentation-associated diseases. Diseases that may be associated with pigmentation traits are skin pigmentation disorders (e.g., albinism, hypopigmentation, hyperpigmentation, vitilgo, lichen simplex chronicus, lamellar ichthyosis, Acanthosis Nigricans, Incontinentia Pigmenti, Liver Spots/Aging Hands, McCune-Albright Syndrome, Moles, Skin Tags, Benign Lentigines, Seborrheic Keratosesmelasma, Progressive Pigmentary Purpura, Tinea Versicolor, Waardenburg Syndrome, or skin cancer). In one embodiment, the disease is skin cancer, e.g., melanoma. Eye pigmentation can be associated with age-related macular degeneration.

Genetic Association to Skin Cancer

Human skin pigmentation pattern is related to susceptibility to skin cancer. Thus, individuals with fair or light skin that burns easily are at increased risk of developing skin cancer, and exposure to the ultraviolet radiation of the sun increases the risk of skin cancer, more so in susceptible individuals with light skin than those with dark skin. It is therefore possible that some variants that are found to be associated with skin pigmentation, in particular those variants that are associated with fair skin that burns easily, and/or the presence of freckles, confer increased susceptibility of developing skin cancer. Indeed, the inventors have discovered that the variant rs6060043 is significantly associated with melanoma cancer (OR=1.39; P=6.1×10⁻⁵; see Example 3 herein). This marker, and markers in linkage disequilibrium therewith, is therefore useful for diagnosing a susceptibility to skin cancer, in particular melanoma, in an individual.

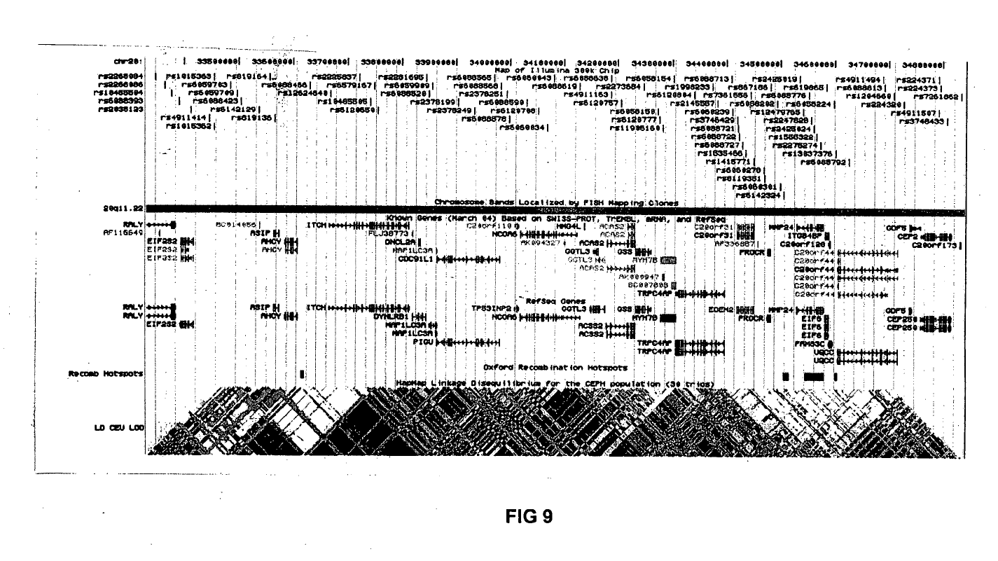

The rs6060043 marker is located within a region of extensive linkage disequilibrium on chromosome 20q11.22 (FIG. 9). Several markers in the region are in strong LD with the marker, as indicated in Table 11 (e.g., markers rs2424994, rs6060009, rs6060017, rs6060025, rs3787223, rs910871, rs3787220, rs6060030, rs1884432, rs6088594, rs6060034, rs6058115, rs6060047, rs7271289, rs2425003, rs17092148, rs11546155, rs17122844 and rs7265992), all of which could be used as surrogates for the marker. The region includes a number of genes, all of which are plausible candidates for being affected by this variant. One of these genes encodes for the Agouti Signaling Protein (ASIP). This gene is the human homologue of the mouse agouti gene which encodes a paracrine signaling molecule that causes hair follicle melanocytes to synthesize pheomelanin, a yellow pigment, instead of the black or brown pigment eumelanin.

Consequently, agouti mice produce hairs with a subapical yellow band on an otherwise black or, brown background when expressed during the midportion of hair growth. The coding region of the human gene is 85% identical to that of the mouse gene and has the potential to encode a protein of 132 amino acids with a consensus signal peptide. The ASIP gene product interacts with the melanocyte receptor for alpha-melanocyte stimulating hormone (MC1R), and in transgenic mice expression of ASIP produced a yellow coat, and expression of ASP in cell culture blocked the MC1R-stimulated accumulation of cAMP in mouse melanoma cells. In mice and humans, binding of alpha-melanocyte-stimulating hormone to the melanocyte-stimulating-hormone receptor (MSHR), the protein product of the melanocortin-1 receptor (MC1R) gene, leads to the synthesis of eumelanin. The ASIP gene therefore is a possible candidate for the observed association of rs6060043 to melanoma and skin and hair pigmentation. The marker is located close to 500 kb distal to the ASIP gene on chromosome 20. It is possible that the marker is in linkage disequilibrium with another marker closer to, or within, the ASIP with functional consequences on gene expression of ASIP, or on the ASIP gene product itself. Alternatively, other the functional effect of rs6060043 is through other genes located in this region.

Follow-up analyses reveal strong association of the AH haplotype with both melanoma (CM) and basal cell carcinoma (BCC) (OR 1.45; P=1.2×10⁻⁹ and 1.35; P=1.2×10⁻⁶, respectively), based on analysis of Icelandic samples and replication cohorts from Sweden and Spain (Example 5). Marker rs1126809 (R402Q) in the TYR gene was also found to associate with risk of CM and BCC (OR 1.21; P=2.8×10⁻⁷ and OR 1.14; P=0.00061, respectively). At the TYRP1 locus, allele C of rs1408799 was found to associate with CM (OR 1.15, P=0.00043). Detail of these results are presented in Example 5 herein.

These results show that certain pigmentation-associated variants that contribute to skin pigmentation traits contribute to risk of CM and BCC, but not others. Moreover, the effect observed for CM and BCC cannot be explained by the effect on the pigmentation trait as defined (see Example 5).

Assessment for Markers and Haplotypes

The genomic sequence within populations is not identical when individuals are compared. Rather, the genome exhibits sequence variability between individuals at many locations in the genome. Such variations in sequence are commonly referred to as polymorphisms, and there are many such sites within each genome For example, the human genome exhibits sequence variations which occur on average every 500 base pairs. The most common sequence variant consists of base variations at a single base position in the genome, and such sequence variants, or polymorphisms, are commonly called Single Nucleotide Polymorphisms (“SNPs”). These SNPs are believed to have occurred in a single mutational event, and therefore there are usually two possible alleles possible at each SNPsite; the original allele and the mutated allele. Due to natural genetic drift and possibly also selective pressure, the original mutation has resulted in a polymorphism characterized by a particular frequency of its alleles in any given population. Many other types of sequence variants are found in the human genome, including microsatellites, insertions, deletions, inversions and copy number variations. A polymorphic microsatellite has multiple small repeats of bases (such as CA repeats, TG on the complimentary strand) at a particular site in which the number of repeat lengths varies in the general population. In general terms, each version of the sequence with respect to the polymorphic site represents a specific allele of the polymorphic site. These sequence variants can all be referred to as polymorphisms, occurring at specific polymorphic sites characteristic of the sequence variant in question. In general terms, polymorphisms can comprise any number of specific alleles. Thus in one embodiment of the invention, the polymorphism is characterized by the presence of two or more alleles in any given population. In another embodiment, the polymorphism is characterized by the presence of three or more alleles. In other embodiments, the polymorphism is characterized by four or more alleles, five or more alleles, six or more alleles, seven or more alleles, nine or more alleles, or ten or more alleles. All such polymorphisms can be utilized in the methods and kits of the present invention, and are thus within the scope of the invention.

In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a trait or disease phenotype).

Alleles for SNP markers as referred to herein refer to the bases A, C, G or T as they occur at the polymorphic site in the SNP assay employed. The allele codes for SNPs used herein are as follows: 1=A, 2=C, 3=G, 4=T. The person skilled in the art will however realise that by assaying or reading the opposite DNA strand, the complementary allele can in each case be measured. Thus, for a polymorphic site (polymorphic marker) characterized by an A/G polymorphism, the assay employed may be designed to specifically detect the presence of one or both of the two bases possible, i.e. A and G. Alternatively, by designing an assay that is designed to detect the opposite strand on the DNA template, the presence of the complementary bases T and C can be measured. Quantitatively (for example, in terms of relative risk), identical results would be obtained from measurement of either DNA strand (+strand or −strand).

Typically, a reference sequence is referred to for a particular sequence. Alleles that differ from the reference are sometimes referred to as “variant” alleles. A variant sequence, as used herein, refers to a sequence that differs from the reference sequence but is otherwise substantially similar. Alleles at the polymorphic genetic markers described herein are variants. Additional variants can include changes that affect a polypeptide. Sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence,. Such sequence changes can alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with a pigmentation trait can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level. The polypeptide encoded by the reference nucleotide sequence is the “reference” polypeptide with a particular reference amino acid sequence, and polypeptides encoded by variant alleles are referred to as “variant” polypeptides with variant amino acid sequences.

A haplotype refers to a segment of DNA that is characterized by a specific combination of alleles, arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various genetic markers.

Detecting specific polymorphic markers and/or haplotypes can be accomplished by methods known in the art for detecting sequences at polymorphic sites. For example, standard techniques for genotyping for the presence of SNPs and/or microsatellite markers can be used, such as fluorescence-based techniques (Chen, X. et al., Genome Res. 9(5): 492-98 (1999)), utilizing PCR, LCR, Nested PCR and other techniques for nucleic acid amplification. Specific methodologies available for SNP genotyping include, but are not limited to, TaqMan genotyping assays and SNPIex platforms (Applied Biosystems), mass spectrometry (e.g., MassARRAY system from Sequenom), minisequencing methods, real-time PCR, Bio-Plex system (BioRad), CEQ and SNPstream systems (Beckman), Molecular Inversion Probe array technology (e.g., Affymetrix GeneChip), BeadArray Technologies (e.g., Illumina GoldenGate and Infinium assays) and the Centaurus platform (Nanogen; see Kutyavin, I.V. et al. Nucleic Acids Research 34, e128 (2006)).

By these or other methods available to the person skilled in the art, one or more alleles at polymorphic markers, including microsatellites, SNPs or other types of polymorphic markers, can be identified.

In certain methods described herein, pigmentation traits or skin cancer risk of a human individual are inferred by determining the presence (or absence) of certain alleles or haplotypes in a nucleic acid sample from the individual. Thus, if at least one specific allele at one or more polymorphic marker or haplotype, or a combination of certain specific alleles at a plurality of markers or haplotypes are identified, the pigmentation traits and/or skin cancer risk for the particular individual can be inferred. Markers and haplotypes found to be predictive (i.e. associated with) particular pigmentation traits are said to be “at-risk” markers or haplotypes for the particular pigmentation trait. In one aspect, the at-risk marker or haplotype is one that confers a significant increased risk (or susceptibility) of the pigmentation trait or skin cancer, i.e. the marker or haplotype is significantly associated with the pigmentation trait or skin cancer. In one embodiment, significance associated with a marker or haplotype is measured by a relative risk (RR). In another embodiment, significance associated with a marker or haplotye is measured by an odds ratio (OR). In a further embodiment, the significance is measured by a percentage. In one embodiment, a significant increased risk is measured as a risk (relative risk and/or odds ratio) of at least 1.2, including but not limited to: at least 1.2, at least 1.3, at least 1.4, at least 1.5, at least 1.6, at least 1.7, 1.8, at least 1.9, at least 2.0, at least 2.5, at least 3.0, at least 4.0, and at least 5.0. In a particular embodiment, a risk (relative risk and/or odds ratio) of at least 1.2 is significant. In another particular embodiment, a risk of at least 1.3 is significant. In yet another embodiment, a risk of at least 1.4 is significant. In a further embodiment, a relative risk of at least about 1.5 is significant. In another further embodiment,a significant increase in risk is at least about 1.7 is significant. However, other cutoffs are also contemplated, e.g. at least 1.15, 1.25, 1.35, and so on, and such cutoffs are also within scope of the present invention. In other embodiments, a significant increase in risk is at least about 20%, including but not limited to about 25%, 30%, 35%, 40%, 45%, 50%, 55%, 60%, 65%, 70%, 75%, 80%, 85%, 90%, 95%, 100%, 150%, 200%, 300%, and 500%. In one particular embodiment, a significant increase in risk is at least 20%. In other embodiments, a significant increase in risk is at least 30%, at least 40%, at least 50%, at least 60%, at least 70%, at least 80%, at least 90% and at least 100%. Other cutoffs or ranges as deemed suitable by the person skilled in the art to characterize the invention are however also contemplated, and those are also within scope of the present invention.

An at-risk polymorphic marker or haplotype of the present invention is one where at least one allele of at least one marker or haplotype is more frequently present in an individual with a particular pigmentation trait or skin cancer, compared to the frequency of its presence in a comparison group (control), and wherein the presence of the marker or haplotype is indicative of susceptibility to the pigmentation trait. The control group may in one embodiment be a population sample, i.e. a random sample from the general population. In another embodiment, the control group is represented by a group of individuals who do not have the particular pigmentation or skin cancer phenotype.

As an example of a simple test for correlation would be a Fisher-exact test on a two by two table. Given a cohort of chromosomes, the two by two table is constructed out of the number of chromosomes that include both of the markers or haplotypes, one of the markers or haplotypes but not the other and neither of the markers or haplotypes.

In other embodiments of the invention, an individual who is at a decreased susceptibility (i.e., at a decreased risk) for a pigmentation trait or skin cancer is an individual in whom at least one specific allele at one or more polymorphic marker or haplotype conferring decreased susceptibility for the pigmentation trait or skin cancer is identified. The marker alleles and/or haplotypes conferring decreased risk are also said to be protective. In one aspect, the protective marker or haplotype is one that confers a significant decreased risk (or susceptibility) of the pigmentation trait or skin cancer. In one embodiment, significant decreased risk is measured as a relative risk of less than 0.9, including but not limited to less than 0.9, less than 0.8, less than 0.7, less than 0.6, less than 0.5, less than 0.4, less than 0.3, less than 0.2 and less than 0.1. In one particular embodiment, significant decreased risk is less than 0.7. In another embodiment, significant decreased risk is less than 0.5. In yet another embodiment, significant decreased risk is less than 0.3. In another embodiment, the decrease in risk (or susceptibility) is at least 20%, including but not limited to at least 25%, at least 30%, at least 35%, at least 40%, at least 45%, at least 50%, at least 55%, at least 60%, at least 65%, at least 70%, at least 75%, at least 80%, at least 85%, at least 90%, at least 95% and at least 98%. In one particular embodiment, a significant decrease in risk is at least about 30%. In another embodiment, a significant decrease in risk is at least about 50%. In another embodiment, the decrease in risk is at least about 70%. Other cutoffs or ranges as deemed suitable by the person skilled in the art to characterize the invention are however also contemplated, and those are also within scope of the present invention.

The person skilled in the art will appreciate that for markers with two alleles present in the population being studied (such as SNPs), and wherein one allele is found in increased frequency in a group of individuals with a pigmentation trait or skin cancer phenotype in the population, compared with controls, the other allele of the marker will be found in decreased frequency in the group of individuals with the pigmentation trait or skin cancer phenotype, compared with controls. In such a case, one allele of the marker (the one found in increased frequency in individuals with the trait) will be the at-risk allele, while the other allele will be a protective allele.

A genetic variant associated with a disease or a trait can be used alone to predict the risk of the disease for a given genotype. For a biallelic marker, such as a SNP, there are 3 possible genotypes: homozygote for the at risk variant, heterozygote, and non carrier of the at risk variant. Risk associated with variants at multiple loci can be used to estimate overall risk. For multiple SNP variants, there are k possible genotypes k=3ⁿ×2^{p}; where n is the number autosomal loci and p the number of gonosomal (sex chromosomal) loci. Overall risk assessment calculations for a plurality of risk variants usually assume that the relative risks of different genetic variants multiply, i.e. the overall risk (e.g., RR or OR) associated with a particular genotype combination is the product of the risk values for the genotype at each locus. If the risk presented is the relative risk for a person, or a specific genotype for a person, compared to a reference population with matched gender and ethnicity, then the combined risk - is the product of the locus specific risk values—and which also corresponds to an overall risk estimate compared with the population. If the risk for a person is based on a comparison to non-carriers of the at risk allele, then the combined risk corresponds to an estimate that compares the person with a given combination of genotypes at all loci to a group of individuals who do not carry risk variants at any of those loci. The group of non-carriers of any at risk variant has the lowest estimated risk and has a combined risk, compared with itself (i.e., non-carriers) of 1.0, but has an overall risk, compare with the population, of less than 1.0. It should be noted that the group of non-carriers can potentially be very small, especially for large number of loci, and in that case, its relevance is correspondingly small.

The multiplicative model is a parsimonious model that usually fits the data of complex traits reasonably well. Deviations from multiplicity have been rarely described in the context of common variants for common diseases, and if reported are usually only suggestive since very large sample sizes are usually required to be able to demonstrate statistical interactions between loci.

By way of an example, let us consider a total of eight variants that have been described to associate with prostate cancer (Gudmundsson, J., et al., Nat Genet 39:631-7 (2007), Gudmundsson, J., et al., Nat Genet 39:977-83 (2007); Yeager, M., et al, Nat Genet 39:645-49 (2007), Amundadottir, L., el al., Nat Genet 38:652-8 (2006); Haiman, C. A., et al., Nat Genet 39:638-44 (2007)). Seven of these loci are on autosomes, and the remaining locus is on chromosome X. The total number of theoretical genotypic combinations is then 3⁷×2¹=4374. Some of those genotypic classes are very rare, but are still possible, and should be considered for overall risk assessment. It is likely that the multiplicative model applied in the case of multiple genetic variant will also be valid in conjugation with non-genetic risk variants assuming that the genetic variant does not clearly correlate with the “environmental” factor. In other words, genetic and non-genetic at-risk variants can be assessed under the multiplicative model to estimate combined risk, assuming that the non-genetic and genetic risk factors do not interact.

Using the same quantitative approach, the combined or overall risk associated with a plurality of variants associated with human pigmentation pattern and skin cancer may be assessed. For example, for predicting skin cancer risk, such plurality of variants is in certain embodiments selected from the group consisting of the All haplotype, marker rs1126809 and marker rs1408799, and markers in linkage disequilibrium therewith. In one preferred embodiment, the plurality of variants comprises the AH haplotype, marker rs1126809 and markers 1408799.

Linkage Disequilibrium

The natural phenomenon of recombination, which occurs on average once for each chromosomal pair during each meiotic event, represents one way in which nature provides variations in sequence (and biological function by consequence). It has been discovered that recombination does not occur randombly in the genome; rather, there are large variations in the frequency of recombination rates, resulting in small regions of high recombination frequency (also called recombination hotspots) and larger regions of low recombination frequency, which are commonly referred to as Linkage Disequilibrium (LD) blocks (Myers, S. et al., Biochem Soc Trans 34:526-530 (2006); Jeffreys, A. J., et al., Nature Genet 29:217-222 (2001); May, C. A., et al., Nature Genet 31:272-275(2002)).

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrance of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurence of each allele or haplotype in the population. For populations of diploids, e.g., human populations, individuals will typically have two alleles for each genetic element (e.g., a marker, haplotype or, gene).

Many different measures have been proposed for assessing the strength of linkage disequilibrium (LD). Most capture the strength of association between pairs of biallelic sites. Two important pairwise measures of LD are r² (sometimes denoted Δ²) and |D′|. Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value can be at least 0.1 such as at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, 0.99 or 1.0. In one preferred embodiment, the significant r² value can be at least 0.2. Alternatively, linkage disequilibrium as described herein, refers to linkage disequilibrium characterized by values of |D′| of at least 0.2, such as 0.3, 0.4, 0.5, 0.6, 0.7, 0.8, 0.85, 0.9, 0.95, 0.96, 0.97, 0.98, 0.99. Thus, linkage disequilibrium represents a correlation between alleles of distinct markers. It is measured by a correlation coefficient r² or |D′| (r² up to 1.0 and |D′| up to 1.0). In certain embodiments, linkage disequilibrium is defined in terms of values for both the r² and |D′| measures. In one such embodiment, a significant linkage disequilibrium is defined as r²>0.1 and |D′|>0.8. In another embodiment, a significant linkage disequilibrium; is defined as r²>0.2 and |D′|>0.8. In another embodiment, a significant linkage disequilibrium is defined as r²>0.2 and |D′|>0.9. Other combinations and permutations of values of r² and |D′|for determining linkage disequilibrium are also possible, and within the scope of the invention. Linkage disequilibrium can be determined in a single human population, as defined herein, or it can be determined in a collection of samples comprising individuals from more than one human population. In one embodiment of the invention, LD is determined in a sample from one or more of the HapMap populations (caucasian (CEU), african (YRI), japanese OPT), chinese (CHB)), as defined (http://www.hapmap.org). In one such embodiment, LD is determined in the CEU population of the HapMap samples. In another embodiment, LD is determined in the YRI population. In yet another embodiment, LD is determined in samples from the Icelandic population.

If all polymorphisms in the genome were independent at the population level, i.e. they segregated independently, then every single one of them would need to be investigated in association studies. However, due to linkage disequilibrium between polymorphisms, tightly linked polymorphisms are strongly correlated, i.e. they tend to be inherited together, which reduces the number of polymorphisms that need to be investigated in an association study to, observe a significant association. Another consequence of LD is that many polymorphisms may give an association signal due to the fact that these polymorphisms are strongly correlated. In practice this means that a large number of identical (or nearly identical) embodiments exist naturally for most markers and haplotypes found to be associated with a particular trait.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, DE et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). More recently, a fine-scale map of recombination rates and corresponding hotspots across the human genome has been generated (Myers, S., et al., Science 310:321-32324 (2005); Myers, S. et al., Biochem Soc Trans 34:526530 (2006)). The map reveals the enormous variation in recombination across the genome, with recombination rates as high as 10-60 cM/Mb in hotspots, while closer to 0 in intervening regions, which thus represent regions of limited haplotype diversity and high LD. The map can therefore be used to define haplotype blocks/LD blocks as genomic regions flanked by recombination hotspots. As used herein, the terms “haplotype block” or “LD block” includes blocks defined by any of the above described characteristics, or other alternative methods used by the person skilled in the art to define such regions.

Some representative methods for identification of haplotype blocks are set forth, for example, in U.S. Published Patent Application Nos. 20030099964, 20030170665, 20040023237 and 20040146870. Haplotype blocks can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association of a particular trait to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given trait, e.g. a pigmentation trait, and as such are useful for use in the methods and kits of the present invention. One or more causative (functional) variants or mutations may reside within the region found to be associating to the pigmentation trait. Such variants may confer a higher relative risk (RR) or odds ratio (OR) than observed for the tagging markers used to detect the association. The present invention thus refers to the markers used for detecting association to the pigmentation trait, as described herein, as well as markers in linkage disequilibrium with the markers. Thus, in certain embodiments of the invention, markers that are in LD with the markers and/or haplotypes of the invention, as described herein, may be used as surrogate markers. In one embodiment, the surrogate markers have relative risk (RR) and/or odds ratio (OR) values identical to the markers or haplotypes initially found to be associating with the pigmentation trait, as described herein; i.e., the surrogate markers are perfect surrogates. The surrogate markers have in another embodiment relative risk (RR) and/or odds ratio (OR) values smaller than for the markers or haplotypes initially found to be associating with the pigmentation trait, as described herein. Such surrogate markers can be used to detect the observed association, and are thus useful in the claimed methods and kits, but may not be perfect surrogates. In other embodiments, the surrogate markers have RR or OR values greater than those initially determined for the markers initially found to be associating with the disease, as described herein. An example of such an embodiment would be a rare, or relatively rare (<10% allelic population frequency) variant in LD with a more common variant (>10% population frequency) initially found to be associating with the pigmentation trait, such as the variants described herein. Identifying and using such markers for detecting the association discovered by the inventors as described herein can be performed by routine methods well known to the person skilled in the art, and are therefore within the scope of the present invention.

Determination of Haplotype Frequency

The frequencies of haplotypes in patient and control groups can be estimated using an expectation-maximization algorithm (Dempster A. et al., J. R. Stat. Soc. 8, 39:1-38 (1977)). An implementation of this algorithm that can handle missing genotypes and uncertainty with the phase can be used. Under the null hypothesis, the patients and the controls are assumed to have identical frequencies. Using a likelihood approach, an alternative hypothesis is tested, where a candidate at-risk-haplotype, which can include the markers described herein, is allowed to have a higher frequency in patients than controls, while the ratios of the frequencies of other haplotypes are assumed to be the same in both groups. Likelihoods are maximized separately under both hypotheses and a corresponding 1-df likelihood ratio statistic is used to evaluate the statistical significance.

To look for at-risk and protective markers and haplotypes within a particular genomic region, association of all possible combinations of genotyped markers is studied, provided those markers span a practical region. The combined patient and control groups can be randomly divided into two sets, equal in size to the original group of patients and controls. The marker and haplotype analysis is then repeated and the most significant p-value registered is determined. This randomization scheme can be repeated, for example, over 100 times to construct an empirical distribution of p-values. In a preferred embodiment, a p-value of <0.05 is indicative of a significant marker and/or haplotype association.

Haplotype Analysis

One general approach to haplotype analysis involves using likelihood-based inference applied to NEsted MOdels (Gretarsdottir S., et al., Nat. Genet. 35:131-38 (2003)). The method is implemented in the program NEMO, which allows for many polymorphic markers, SNPs and microsatellites. The method and software are specifically designed for case-control studies where the purpose is to identify haplotype groups that confer different risks. It is also a tool for studying LD structures. In NEMO, maximum likelihood estimates, likelihood ratios and p-values are calculated directly, with the aid of the EM algorithm, for the observed data treating it as a missing-data problem.

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. The information measure for haplotype analysis is described in Nicolae and Kong (Technical Report 537, Department of Statistics, University of Statistics, University of Chicago; Biometrics, 60(2):368-75 (2004)) as a natural extension of information measures defined for linkage analysis, and is implemented in NEMO.

For single marker association to a trait, the Fisher exact test can be used to calculate two-sided p-values for each individual allele. Usually, all p-values are presented unadjusted for multiple comparisons unless specifically indicated. The presented frequencies (for microsatellites, SNPs and haplotypes) are allelic frequencies as opposed to carrier frequencies. To minimize any bias due the relatedness of the patients who were recruited as families for the linkage analysis, first and second-degree relatives can be eliminated from the patient list. Furthermore, the test can be repeated for association correcting for any remaining relatedness among the case (i.e., those with a particular pigmentation trait) and control groups, by extending a variance adjustment procedure described in Risch, N. & Teng, J. (Genome Res., 8:1273-1288 (1998)), DNA pooling (ibid) for sibships so that it can be applied to general familial relationships, and present both adjusted and unadjusted p-values for comparison. The differences are in general very small as expected. To assess the significance of single-marker association corrected for multiple testing we can carry out a randomization test using the same genotype data. Cohorts of cases and controls can be randomized and the association analysis redone multiple times (e.g., up to 500,000 times) and the p-value is the fraction of replications that produced a p-value for some marker allele that is lower than or equal to the p-value we observed using the original case and control cohorts.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fᵢ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

Linkage Disequilibrium Using NEMO

LD between pairs of markers can be calculated using the standard definition of D′ and r² (Lewontin, R., Genetics 49:49-67 (1964); Hill, W. G. & Robertson, A. Theor. Appl. Genet. 22:226-231 (1968)). Using NEMO, frequencies of the two marker allele combinations are estimated by maximum likelihood and deviation from linkage equilibrium is evaluated by a likelihood ratio test. The definitions of D′ and r² are extended to include microsatellites by averaging over the values for all possible allele combination of the two markers weighted by the marginal allele probabilities. When plotting all marker combination to elucidate the LD structure in a particular region, we plot D′ in the upper left corner and the p-value in the lower right corner. In the LD plots the markers can be plotted equidistant rather than according to their physical location, if desired.

Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

As described herein, certain polymorphic markers and haplotypes comprising such markers are found to be useful for inferring pigmentation traits and for predicting susceptibility to skin cancer in human individuals. Risk assessment for the pigmentation traits involves the use of the markers or haplotypes for inferring the most likely pigmentation trait of the individual. Particular alleles of polymorphic markers are found more frequently in individuals with the pigmentation trait, than in individuals without the pigmentation trait. Particular alleles of polymorphic markers are also found more frequently in individuals with, or at risk for, a skin cancer, than in individuals that are not at risk for, or have not developed, the skin cancer. Therefore, these marker alleles have predictive value for determining risk of pigmentation traits and/or skin cancer, or for inferring pigmentation traits, in an individual. Tagging markers within regions of high linkage disequilibrium, such as haplotype blocks or LD blocks comprising at-risk markers (i.e., markers predictive of the pigmentation trait), such as the markers of the present invention, can be used as surrogates for other markers and/or haplotypes within the haplotype block or LD block.

Such surrogate markers can be located within a particular haplotype block region or LD block region. Such surrogate markers can also sometimes be located outside the physical boundaries of such a haplotype block or LD block, either in close vicinity of the LD block/haplotype block, but possibly also located in a more distant genomic location.

Long-distance LD can for example arise if particular genomic regions (e.g., genes) are in a functional relationship. For example, if two genes encode proteins that play a role in a shared metabolic pathway, then particular variants in one gene may have a direct impact on observed variants for the other gene. Let us consider the case where a variant in one gene leads to increased expression of the gene product. To counteract this effect and preserve overall flux of the particular pathway, this variant may have led to selection of one (or more) variants at a second gene that conferes decreased expression levels of that gene. These two genes may be located in different genomic locations, possibly on different chromosomes, but variants within the genes are in apparent LD, not because of their shared physical location within a region of high

LD, but rather due to evolutionary forces. Such LD is also contemplated and within scope of the present invention. The skilled person will appreciate that many other scenarios of functional gene-gene interaction are possible, and the particular example discussed here represents only one such possible scenario.

Markers with values of r² equal to 1 are perfect surrogates for the at-risk variants, i.e. genotypes for one marker perfectly predicts genotypes for the other. Markers with smaller values of r² than 1 can also be surrogates for the at-risk variant, or alternatively represent variants with relative risk values as high as or possibly even higher than the at-risk variant. The at-risk variant identified may not be the functional variant itself, but is in this instance in linkage disequilibrium with the true functional variant. The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation genotype surrogate markers in linkage disequilibrium with the markers and/or haplotypes as described herein. Examples of surrogate markers of the markers and haplotypes, of the present invention are provided in the Examples herein. The tagging or surrogate markers in LD with the at-risk variants detected, also have predictive value for the pigmentation trait and/or the skin cancer, or a susceptibility to the pigmentation trait and/or skin cancer, in an individual.

The present invention can in certain embodiments be practiced by assessing a sample comprising genomic DNA from an individual for the presence of variants described herein to be associated with skin cancer, or useful for predicting pigmentation traits. Such assessment typically steps that detect the presence or absence of at least one allele of at least one polymorphic marker, using methods well known to the skilled person and further described herein, and based on the outcome of such assessment, determine whether the individual from whom the sample is derived is at increased or decreased risk (increased or decreased susceptibility) of the skin cancer or pigmentation trait. Detecting particular alleles of polymorphic markers can in certain embodiments be done by obtaining nucleic acid sequence data about a particular human individual, that identifies at least one allele of at least one polymorphic marker. Different alleles of the at least one marker are associated with different susceptibility to the disease in humans. Obtaining nucleic acid sequence data can comprise nucleic acid sequence at a single nucleotide position, which is sufficient to identify alleles at SNPs. The nucleic acid sequence data can also comprise sequence at any other number of nucleotide positions, in particular for genetic markers that comprise multiple nuclotide positions; and can be anywhere from two to hundreds of thousands, possibly even millions, of nucleotides (in particular, in the case of copy number variations (CNVs)).

In certain embodiments, the invention can be practiced utilizing a dataset comprising information about the genotype status of at least one polymorphic marker associated with a disease or pigmentation trait (or markers in linkage disequilibrium with at least one marker associated with the disease or pigmentation trait). In other words, a dataset containing information about such genetic status, for example in the form of genotype counts at a certain polymorphic marker, or a plurality of markers (e.g., an indication of the presence or absence of certain at-risk alleles), or actual genotypes for one or more markers, can be queried for the presence or absence of certain at-risk alleles at certain polymorphic markers shown by the present inventors to be associated with the disease. A positive result for a variant (e.g., marker allele) associated with the disease or trait, is indicative of the individual from which the dataset is derived is at increased susceptibility (increased risk) of the disease or trait.

In certain embodiments of the invention, a polymorphic marker is correlated to a disease by referencing genotype data for the polymorphic marker to a look-up table that comprises correlations between at least one allele of the polymorphism and the disease. In some embodiments, the table comprises a correlation for one polymorhpism. In other embodiments, the table comprises a correlation for a plurality of polymorhpisms. In both scenarios, by referencing to a look-up table that gives an indication of a correlation between a marker and the disease, a risk for the disease, or a susceptibility to the disease, can be identified in the individual from whom the sample is derived. In some embodiments, the correlation is reported as a statistical measure. The statistical measure may be reported as a risk measure, such as a relative risk (RR), an absolute risk (AR) or an odds ratio (OR).

Certain markers and haplotypes described herein, e.g., the markers presented in Table 10 and Table 11, may be useful for risk assessment of, and/or inferring, certain pigmentation traits, either alone or in combination. Certain markers, e.g. markers as presented in 21, 22 and 23, may also be useful for risk assessment of skin cancer, alone or in combination. As exemplified herein, even in cases where the increase in risk by individual markers is relatively modest, i.e. on the order of 10-30%, the association may have significant implications. Thus, relatively common variants may have significant contribution to the overall risk (Population Attributable Risk is high), or combination of markers can be used to define groups of individual who, based on the combined risk of the markers, are likely to be characterized by a particular pigmentation trait or at risk for a skin cancer, i.e. the combination of markers and/or haplotypes may be used for inferring the pigmentation trait, or predict the skin cancer, of the individual.

Thus, in certain embodiments of the invention, a plurality of variants (genetic markers and/or haplotypes) is used for inferring a pigmentation trait or determine susceptibility of a skin cancer. These variants are in one embodiment selected from the variants as disclosed herein. Other embodiments include the use of the variants of the present invention in combination with other variants known to be useful for inferring pigmentation traits or predict risk of skin cancer, as known to those skilled in the art and described in published documents. In such embodiments, the genotype status of a plurality of markers and/or haplotypes is determined in an individual, and the status of the individual compared with the population frequency of the associated variants, to determine the likelihood of a skin cancer, or infer a particular pigmentation trait in the individual. Methods known in the art, such as multivariate analyses or joint risk analyses, may subsequently be used to determine the overall risk conferredbased on the genotype status at the multiple loci. Assessment of risk based on such analysis may subsequently be used in the methods and kits of the invention, as described herein. In one preferred embodiment, a first set of a plurality of samples from individuals with certain pigmentation traits (discovery sample) is used to create prediction rules for other samples. For example, in a generalized linear model, a pigmentation trait (such as eye color or hair color) can be treated as a categorical response with a plurality of categories and genotypes at all associated sequence variants can be used as covariates, to model the pigmentation trait. Another example is provided by a two step model, in which the first step involves predicting a certain pigmentation trait based solely on one variant or a set of variants. The second step involves modeling other pigmentation traits as an ordinal variable the additional pigmentation traits between the predefined extremes of pigmentation, such as blond and brown or black hair.

As described in the above, the haplotype block structure of the human genome has the effect that a large number of variants (markers and/or haplotypes) in linkage disequilibrium with the variant originally associated with a trait, such as a pigmentation trait, may be used as surrogate markers for assessing association to the trait. The number of such surrogate markers will depend on factors such as the historical recombination rate in the region, the mutational frequency in the region (i.e., the number of polymorphic sites or markers in the region), and the extent of LD (size of the LD block) in the region. These markers are usually located within the physical boundaries of the LD block or haplotype block in question as defined using the methods described herein, or by other methods known to the person skilled in the art. However, sometimes marker and/or haplotype association is found to extend beyond the physical boundaries of the haplotype block as defined. This may occur, for example, if the association signal resides on an old haplotype background which has subsequently undergone recombination, so as to separate observed association signals into separate apparent LD blocks. Such markers and/or haplotypes may in those cases be also used as surrogate markers and/or haplotypes for the markers and/or haplotypes physically residing within the haplotype block as defined. As a consequence, markers and haplotypes in LD (typically characterized by r² greater than 0.1, such as r² greater than 0.2, including r² greater than 0.3, also including r² greater than 0.4) with the markers and haplotypes of the present invention are also within the scope of the invention, even if they are physically located beyond the boundaries of the haplotype block as defined. This includes markers that are described herein (e.g., Tables 10, 14, 25 and 26; SEQ ID NO:1-138), but may also include other markers that are in strong LD (e.g., characterized by r² greater than 0.1, such as r² greater than 0.2, including r² greater than 0.3, 0.4, 0.5, 0.6, 0.7, 0.8 or 0.9 and/or |D′|>0.8, including |D′|>0.9) with one or more of the markers listed in Tables 10, 14, 25 and 26.

For the SNP markers described herein, the opposite allele to the allele found to be in excess in patients with a particular skin cancer, or in individuals with a particular pigmentation trait (at-risk allele) is found in decreased frequency in such individuals. Such marker alleles, and/or haplotypes comprising such alleles, are thus protective for the skin cancer or pigmentation trait, i.e. they confer a decreased risk or susceptibility of individuals carrying these markers and/or haplotypes developing the skin cancer of the pigmentation trait.

Certain variants of the present invention, including certain haplotypes comprise, in some cases, a combination of various genetic markers, e.g., SNPs and microsatellites. Detecting haplotypes, can be accomplished by methods known in the art and/or described herein for detecting sequences at polymorphic sites. Furthermore, correlation between certain haplotypes or sets of markers and disease phenotype can be verified using standard techniques. A representative example of a simple test for correlation would be a Fisher-exact test on a two by two table.

In specific embodiments, a marker allele or haplotype found to be associated with a pigmentation trait or skin cancer, is one in which the marker allele or haplotype is more frequently present in an individual with a particular trait or disease(e.g., pigmentation or skin cancer) (affected), compared to the frequency of its presence in an individual who does not have the particular trait or disease (control), wherein the presence of the marker allele or haplotype is indicative of the trait or disease, or a susceptibility to the trait or disease. In other embodiments, at-risk markers in linkage disequilibrium with one or more markers found to be associated with a trait or disease are tagging or surrogate markers that are more frequently present in an individual with a particular pigmentation trait or skin cancer (affected), compared to the frequency of their presence in individuals who do not have the pigmentation trait or the skin cancer (control), wherein the presence of the tagging markers is indicative of increased susceptibility or risk of the particular pigmentation trait and/or skin cancer.

Study Population

In a general sense, the methods and kits of the invention can be utilized on samples containing genomic DNA from any source, i.e. from any individual and any kind of sample that contains genomic DNA. In preferred embodiments, the individual is a human individual. The individual can be an adult, child, or fetus. The present invention also provides for assessing markers and/or haplotypes in individuals who are members of a particular target population. Such a target population is in one embodiment one or several individuals that are to be investigated for one, or several, pigmentation traits. This group of individuals can for example be represented by a genomic DNA sample obtained from the scene of a crime or a natural disaster, as further described herein.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Styrkarsdottir, U., et al. N Engl J Med Apr. 29, 2008 (Epub ahead of print); Thorgeirsson, T., et al. Nature 452:638-42 (2008); Gudmundsson, J., et al. Nat Genet. 40:281-3 (2008); Stacey, S. N., et al., Nat Genet. 39:865-69 (2007); Helgadottir, A., et al., Science 316:1491-93 (2007); Steinthorsdottir, V., et al., Nat Genet. 39:770-75 (2007); Gudmundsson, J., et al., Nat Genet. 39:631-37 (2007); Frayling, T M, Nature Reviews Genet 8:657-662 (2007); Amundadottir, L. T., et al., Nat Genet. 38:652-58 (2006); Grant, S. F., et al., Nat Genet. 38:320-23 (2006)). Thus, genetic findings in the Icelandic population have in general been replicated in other populations, including populations from Africa and Asia.

It is thus believed that the markers of the present invention found to be associated with pigmentation traits and/or skin cancer will show similar association in other human populations Particular embodiments comprising individual human populations are thus also contemplated and within the scope of the invention. Such embodiments relate to human subjects that are from one or more human population including, but not limited to, Caucasian populations, European populations, American populations, Eurasian populations, Asian populations, Central/South Asian populations, East Asian populations, Middle Eastern populations, African populations, Hispanic populations, and Oceanian populations. European populations include, but are not limited to, Swedish, Norwegian, Finnish, Russian, Danish, Icelandic, Irish, Kelt, English, Scottish, Dutch, Belgian, French, German, Spanish, Portugues, Italian, Polish, Bulgarian, Slavic, Serbian, Bosnian, Czech, Greek and Turkish populations. The invention furthermore in other embodiments can be practiced in specific human populations that include Bantu, Mandenk, Yoruba, San, Mbuti Pygmy, Orcadian, Adygel, Russian, Sardinian, Tuscan, Mozabite, Bedouin, Druze, Palestinian, Balochi, Brahui, Makrani, Sindhi, Pathan, Burusho, Hazara, Uygur, Kalash, Han, Dai, Daur, Hezhen, Lahu, Miao, Orogen, She, Tujia, Tu, Xibo, Yi, Mongolan, Naxi, Cambodian, Japanese, Yakut, Melanesian, Papuan, Karitianan, Surui, Colmbian, Maya and Pima.

The racial contribution in individual subjects may also be determined by genetic analysis. Genetic analysis of ancestry may be carried out using unlinked microsatellite markers such as those set out in Smith et al. (Am J Hum Genet 74, 1001-13 (2004)).

In certain embodiments, the invention relates to markers and/or haplotypes identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, are polymorphic in one population but not in another. The person skilled in the art will however apply the methods available and as thought herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

Utility for Forensic Testing

Human pigmentation pattern, in particular hair, eye and skin pigmentation are amongst the most visible examples of human phenotypic variation. Most individuals can be characterized by these traits, making them particularly useful for describing the overall appearance of an individual. The pigmentation variants described herein can thus be used for describing the overall appearance of any particular human individual, as long as a sample containing genomic DNA from the individual is available. These characteristics can be used to aid in the identification of individuals, for example by selection from a small population of individuals, i.e. a. group of individuals. The variants of the invention can alternatively be used to place individuals with specific pigmentation characteristics into subgroups, each of which is characterized by a certain combination of hair, eye and/or skin pigmentation pattern or colour. Although the vast majority of variation in human eye and hair color is found among individuals of European ancestry, with most other human populations fixed for brown eyes and black hair, determination of pigmentation by the genetic methods described herein does not require assumption or knowledge of race. Some non-limiting examples of how determination of pigmentation pattern can be applied include:

Crime analysis. Frequently, samples containing DNA are obtained from the scene of the crime or other sources in a crime investigation. Analysis of such samples can be used for describing the individual from which the sample originates, aiding in the identification of a potential criminal or a suspect, either by limiting a list of possible suspects or aiding in the actual identification from a pool of possible suspects.

Natural disasters frequently render the victim unrecognizable by visual inspection. Analysis of the pigmentation pattern based on genetic material can be used to define the appearance of the individual, which can be used to aid in the identification of the individual from which the sample originates.

Certain pigmentation characteristics may be more useful than others in certain settings, depending on the scenario. For example, it may be extremely informative to know that an individual from which a sample is obtained, is likely to have a specific hair color, such as red hair, or having a characteristic skin appearance, such as freckles. This may find particular use in crime research, wherein several indications are ultimately used to identify the most likely suspects.

Combination with other known Genetic Tests

The genetic variants of the invention can be used either alone, in combination with other genetic variants described herein, or in combination with other genetic variants commonly used to characterize individuals. Examples of such additional variants includes ABO blood groups, other blood groups, tissue typing, tandem repeats (STR), or any other genetic variants that are commonly used to characterize humans. Other variants that may be useful with the variants of the present invention include variants that are associated with other human characteristics, such as facial appearance, size and/or number of teeth, ear shape, baldness, height, weight, body mass (such as body mass index, BMI), or any other variant that is associated with human appearance. The invention may furthermore be practiced by combination with methods for determining human ancestry. For example, genetic analysis of ancestry may be carried out using unlinked microsatellite markers such as those set out in Smith et al. (Am J Hum Genet 74, 1001-13 (2004)).

Furthermore, the variants of the present invention may be useful in combination with variants that are associated with human health traits, in particular various human diseases. This includes both diseases leading to specific physical appearance and diseases mainly affecting the internal organs. Such variants can be Mendelian in nature (i.e., predict the phenotype in a strictly Mendelian fashion), or they are associated with the phenotype in a more complex interaction with other genetic variants and/or environmental factors.

Utility of Genetic Testing

The person skilled in the art will appreciate and understand that the variants described herein in general do not, by themselves, provide an absolute identification of individuals who will develop a particular form of cancer. The variants described herein do however indicate increased and/or decreased likelihood that individuals carrying the at-risk or protective variants of the invention will develop a cancer such as CM, BCC and/or SCC. This information is however extremely valuable in itself, as outlined in more detail in the below, as it can be used to, for example, initiate preventive measures at an early stage, perform regular physical and/or mental exams to monitor the progress and/or appearance of symptoms, or to schedule exams at a regular interval to identify early symptoms, so as to be able to apply treatment at an early stage.

Genetic Testing for Melanoma. Relatives of melanoma patients are themselves at increased risk of melanoma, suggesting an inherited predisposition [Amundadottir, et al., (2004), PLoS Med, 1, e65. Epub 2004 Dec. 28.]. A series of linkage based studies implicated CDKN2a on 9p21 as a major CM susceptibility gene [Bataille, (2003), Eur J Cancer, 39, 1341-7.]. CDK4 was identified as a pathway candidate shortly afterwards, however mutations have only been observed in a few families worldwide [Zuo, et al., (1996), Nat Genet, 12, 97-9.]. CDKN2a encodes the cyclin dependent kinase inhibitor p16 which inhibits CDK4 and CDK6, preventing G1-S cell cycle transit. An alternate transcript of CKDN2a produces p14ARF, encoding a cell cycle inhibitor that acts through the MDM2-p53 pathway. It is likely that CDKN2a mutant melanocytes are deficient in cell cycle control or the establishment of senescence, either as a developmental state or in response to DNA damage. Overall penetrance of CDKN2a mutations in familial CM cases is 67% by age 80. However penetrance is increased in areas of high melanoma prevalence [Bishop, et al., (2002), J Natl Cancer Inst, 94, 894-903].

Individual who are at increased risk of melanoma might be offered regular skin examinations to identify incipient tumours, and they might be counselled to avoid excessive UV exposure. Chemoprevention either using sunscreens or pharmaceutical agents [Bowden, (2004), Nat Rev Cancer, 4, 23-35.] might be employed. For individuals who have been diagnosed with melanoma, knowledge of the underlying genetic predisposition may be useful in determining appropriate treatments and evaluating risks of recurrence and new primary tumours.

Endogenous host risk factors for CM are in part under genetic control. It follows that a proportion of the genetic risk for CM resides in the genes that underpin variation in pigmentation and nevi. The Melanocortin 1 Receptor (MC1R) is a G-protein coupled receptor involved in promoting the switch from pheomelanin to eumelanin synthesis. Numerous, well characterized variants of the MC1R gene have been implicated in red haired, pale skinned and freckle prone phenotypes. We and others have demonstrated the MC1R variants confer risk of melanoma (Gudbjartsson et. al., Nature Genetics, in press). Other pigmentation trait-associated variants, in the ASIP, TYR and TYRP1 genes have also been implicated in melanoma risk (Gudbjartsson et. al., Nature Genetics, in press). ASIP encodes the agouti signalling protein, a negative regulator of the melanocortin 1 receptor. TYR and TYRP1 are enzymes involved in melanin synthesis and are regulated by the MC1R pathway. Individuals at risk for BCC and/or SCC might be offered regular skin examinations to identify incipient tumours, and they might be counselled to avoid excessive UV exposure. Chemoprevention either using sunscreens or pharmaceutical agents [Bowden, (2004), Nat Rev Cancer, 4, 23-35.] might, be employed. For individuals who have been diagnosed with BCC or SCC, knowledge of the underlying genetic predisposition may be useful in determining appropriate treatments and evaluating risks of recurrence and new primary tumours. Screening for susceptibility to BCC or SCC might be important in planning the clinical management of transplant recipients and other immunosuppressed individuals.

Genetic Testing for Basal Cell Carcinoma and Squamous Cell Carcinoma. A positive family histoy is a risk factor for SCC and BCC [Hemminki, et al., (2003), Arch Dermatol, 139, 885-9; Vitasa, et al., (1990), Cancer, 65, 2811-7] suggesting an inherited component to the risk of BCC and/or SCC. Several rare genetic conditions have been associated with increased risks of BCC and/or SCC, including Nevoid Basal Cell Syndrome (Gorlin's Syndrome), Xeroderma Pigmentosum (XP), and Bazex's Syndrome. XP is underpinned by mutations in a variety of XP complementation group genes. Gorlin's Syndrome results from mutations in the PTCH1 gene. In addition, variants in the CYP2D6 and GSTT1 genes have been associated with BCC [Wong, et al., (2003), Bmj, 327, 794-8]. Polymorphisms in numerous genes have been associated with SCC risk.

Fair pigmentation traits are known risk factors for BCC and/or SCC and are thought act, at least in part, through a reduced protection from UV irradiation. Thus, genes underlying these fair pigmentation traits have been associated with risk. MC1R, ASIP, and TYR have been shown to confer risk for SCC and/or BCC (Gudbjartsson et. al., (2008) Nat Genet 40(7), 703-706) [Bastiaens, et al., (2001), Am J Hum Genet, 68, 884-94; Han, et al., (2006), Int J Epidemiol, 35, 1514-21]. However, pigmentation characteristics do not completely account for the effects of MC1R, ASIP and TYR variants. This may be because self-reported pigmentation traits do not adequately reflect those aspects of pigmentation status that relate best to skin cancer risk. It amy also indicate that MC1R, ASIP and TYR have risk-associated functions that are not directly related to easily observable pigmentation traits (Gudbjartsson et. al., Nature Genetics, in press) [Rees, (2006), J Invest Dermatol, 126, 1691-2]. This indicates that genetic testing for pigmentation trait associated variants may have increased utility in BCC and/or SCC screening over and above what can be obtained from observing patients' pigmentation phenotypes.

Diagnostic and Screening Methods

The present invention provides methods of inferring at least one pigmentation trait of a human individual, by determining the identity of at least one allele of at least one polymorphic marker in a nucleic acid sample from the individual, as described in detail herein, wherein the presence of the at least one allele is indicative of at least one pigmentation trait of the individual. The markers that are preferably used in the methods of the invention include the markers listed in Table 10 (SEQ ID NO:1-SEQ ID NO:134), and markers in linkage disequilibrium therewith (e.g., as provided in Table 11 herein). The invention furthermore provides markers and haplotypes for determining suscepbility to skin cancers, e.g. as provided in the Exemplification herein, e.g. the markers and haplotypes provided in tables 21-26 herein, e.g., the markers with sequence as set forth in SEQ ID NO:135-483 herein. The markers in linkage disequilibrium include in one embodiment markers with values of the LD measures r² of greater than 0.2 and/or |D′| of greater than 0.8. Other cutoff values of these LD measures are however also contemplated, as described in detail herein. The particular markers or haplotypes that have been found to be correlated with certain pigmentation traits and/or skin cancer, and therefore are useful for inferring pigmentation traits and/or skin cancer for a human individual, are those that are significantly associated with, i.e. conferring a significant risk of, the particular pigmentation traits and skin cancer. In certain embodiments, the significance of association of the at least one marker allele or haplotype is characterized by a p value<0.05. In other embodiments, the significance of association is characterized by smaller (i.e., more significant) p-values, such as p<0.01, p<0.001, p<0.0001, p<0.00001, p<0.000001, p<0.0000001, p<0.00000001 or p<0.000000001.

The present invention pertains in some embodiments to methods of clinical applications of diagnosis, e.g., diagnosis performed by a medical professional. In other embodiments, the invention pertains to methods of diagnosis or determination of a susceptibility performed by a layman. The layman can be the customer of a genotyping service. The layman may also be a genotype service provider, who performs genotype analysis on a DNA sample from an individual, in order to provide service related to genetic risk factors for particular traits or diseases, based on the genotype status of the individual (i.e., the customer). Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs simultaneously, at relatively little cost. The resulting genotype information, which can be made available to the individual, can be compared to information about disease or trait risk associated with various SNPs, including information from public litterature and scientific publications. The diagnostic application of disease-associated alleles as described herein, can thus for example be performed by the individual, through analysis of his/her genotype data, by a health professional based on results of a clinical test, or by a third party, including the genotype service provider. The third party may also be service provider who interprets genotype information from the customer to provide service related to specific genetic risk factors, including the genetic markers described herein. In other words, the diagnosis or determination of a susceptibility of genetic risk can be made by health professionals, genetic counselors, third parties providing genotyping service, third parties providing risk assessment service or by the layman (e.g., the individual), based on information about the genotype status of an individual and knowledge about the risk conferred by particular genetic risk factors (e.g., particular SNPs). In the present context, the term “diagnosing”, “diagnose a susceptibility” and “determine a susceptibility” is meant to refer to any available diagnostic method, including those mentioned above.

In certain embodiments, a sample containing genomic DNA from an individual is collected. Such sample can for example be a buccal swab, a saliva sample, a blood sample, or other suitable samples containing genomic DNA, as described further herein. The genomic DNA is then analyzed using any common technique available to the skilled person, such as high-throughput array technologies. Results from such genotyping are stored in a convenient data storage unit, such as a data carrier, including computer databases, data storage disks, or by other convenient data storage means. In certain embodiments, the computer database is an object database, a relational database or a post-relational database. The genotype data is subsequently analyzed for the presence of certain variants known to be susceptibility variants for a particular human conditions, such as the genetic variants described herein. Genotype data can be retrieved from the data storage unit using any convenient data query method. Calculating risk conferred by a particular genotype for the individual can be based on comparing the genotype of the individual to previously determined risk (expressed as a relative risk (RR) or and odds ratio (OR), for example) for the genotype, for example for an heterozygous carrier of an at-risk variant for a particular disease or trait (such as skin cancer or a pigmentation trait). The calculated risk for the individual can be the relative risk for a person, or for a specific genotype of a person, compared to the average population with matched gender and ethnicity. The average population risk can be expressed as a weighted average of the risks of different genotypes, using results from a reference population, and the appropriate calculations to calculate the risk of a genotype group relative to the population can then be performed. Alternatively, the risk for an individual is based on a comparison of particular genotypes, for example heterozygous carriers of an at-risk allele of a marker compared with non-carriers of the at-risk allele. Using the population average may in certain embodiments be more convenient, since it provides a measure which is easy to interpret for the user, i.e. a measure that gives the risk for the individual, based on his/her genotype, compared with the average in the population. The calculated risk estimated can be made available to the customer via a website, preferably a secure website.

In certain embodiments, a service provider will include in the provided service all of the steps of isolating genomic DNA from a sample provided by the customer, performing genotyping of the isolated DNA, calculating genetic risk based on the genotype data, and report the risk to the customer. In some other embodiments, the service provider will include in the service the interpretation of genotype data for the individual, i.e., risk estimates for particular genetic variants based on the genotype data for the individual. In some other embodiments, the service provider may include service that includes genotyping service and interpretation of the genotype data, starting from a sample of isolated DNA from the individual (the customer).

Overall risk for multiple risk variants can be performed using standard methodology. For example, assuming a multiplicative model, i.e. assuming that the risk of individual risk variants multiply to establish the overall effect, allows for a straight-forward calculation of the overall risk for multiple markers.

The diagnostic methods in which the markers of the invention are useful involve detecting the presence or absence of at least allele of at least one marker, or at least one haplotype, that is associated with at least one pigmentation trait or skin cancer. The methods are useful for inferring a particular pigmentation trait or skin cancer of a human individual, by assessing the presence of a particular allele of at least one polymorphic marker, and comparing that with the frequency of the allele in a reference population. If the sample from the individual contains an allele of a polymorphic marker that is associated with a particular pigmentation trait or skin cancer, i.e. the allele occurs commonly in individuals with that particular trait, then there is a particular likelihood that the individual in question can be characterized by that particular pigmentation trait, or that the individual will develop the skin cancer. Analyzing a plurality of polymorphic markers can allow for a more rigorous assessment of the presence or absence of a particular pigmentation trait, by measuring several polymorphic markers that are associated with the trait. Alternatively, the analysis of a plurality of markers associated with a variety of pigmentation trait can allow the assessment of a plurality of pigmentation traits in the individual. In other words, the pigmentation traits can be inferred from the measurements of polymorphic markers that are associated with the trait. In some embodiments, as further described herein, particular variants (i.e. particular alleles at particular polymorphic markers) are associated with more than one pigmentation trait. Thus, by determining the presence or absence of such variants can be used to infer more than one pigmentation trait simultaneously.

The haplotypes described herein include combinations of alleles at various genetic markers (e.g., SNPs, microsatellites). The detection of the particular genetic marker alleles that make up the particular haplotypes can be performed by a variety of methods described herein and/or known in the art. For example, genetic markers can be detected at the nucleic acid level (e.g., by direct nucleotide sequencing or by other means known to the skilled in the art) or at the amino acid level if the genetic marker affects the coding sequence of a protein encoded by the nucleic acid (e.g., by protein sequencing or by immunoassays using antibodies that recognize such a protein). The marker alleles or haplotypes of the present invention correspond to fragments of a genomic DNA segment associated with at least one pigmentation trait or skin cancer. Such fragments encompass the DNA sequence of the polymorphic marker or haplotype in question, but may also include DNA segments in strong LD (linkage disequilibrium) with the marker or haplotype. In one embodiment, such segments comprises segments in LD with the marker or haplotype as determined by a value of r² greater than 0.2 and/or |D′|>0.8).

In one embodiment, analysis of polymorphic markers, as described herein, can be accomplished using hybridization methods, such as Southern analysis, Northern analysis, and/or in situ hybridizations (see Current Protocols in Molecular Biology, Ausubel, F. et al., eds., John Wiley & Sons, including all supplements). A biological sample from an individual (a “test sample”) containing genomic DNA, RNA, or cDNA is obtained. The subject can be an adult, child, or fetus. The test sample can be from any source that contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined. The presence of a specific marker allele can be indicated by sequence-specific hybridization of a nucleic acid probe specific for the particular allele. The presence of more than specific marker allele or a specific haplotype can be indicated by using several sequence-specific nucleic acid probes, each being specific for a particular allele. In one embodiment, a haplotype can be indicated by a single nucleic acid probe that is specific for the specific haplotype (i.e., hybridizes specifically to a DNA strand comprising the specific marker alleles characteristic of the haplotype). A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample.

To assess for the presence of specific alleles at polymorphic markers, a hybridization sample is formed by contacting the test sample containing a DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length that is sufficient to specifically hybridize under stringent conditions to appropriate mRNA or genomic DNA. For example, the nucleic acid probe can comprise all or a portion of the nucleotide sequence flanking at least one of the polymorphic markers listed in Tables 10, 11, 14, 25 and 26 as described herein, optionally comprising at least one allele of a marker described herein, or at least one haplotype described herein, or the probe can be the complementary sequence of such a sequence. In a particular embodiment, the nucleic acid probe is a portion of the nucleotide sequence flanking a polymorphic marker as described herein, optionally comprising at least one allele of the marker, or at least one allele of one polymorphic marker or haplotype comprising at least two polymorphic markers described herein, or the probe can be the complementary sequence of such a sequence. Other suitable probes for use in the diagnostic assays of the invention are described herein. Hybridization can be performed by methods well known to the person skilled in the art (see, e.g., Current Protocols in Molecular Biology, Ausubel, F. et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe. The process can be repeated for any markers of the present invention, or markers that make up a haplotype of the present invention, or multiple probes can be used concurrently to detect more than one marker alleles at a time. It is also within the scope of the invention to design a single probe containing more than one marker alleles of a particular haplotype (e.g., a probe containing alleles complementary to 2, 3, 4, 5 or all of the markers that make up a particular haplotype). Detection of the particular markers of the haplotype in the sample is indicative that the source of the sample has the particular haplotype (e.g., a haplotype) and therefore is likely to be characterized by a specific pigmentation trait.

In another hybridization method, Northern analysis (see Current Protocols in Molecular Biology, Ausubel, F. et al., eds., John Wiley & Sons, supra) is used to identify the presence of specific alleles of polymorphic markers associated with a pigmentation trait. For Northern analysis, a test sample of RNA is obtained from the subject by appropriate means. As described herein, specific hybridization of a nucleic acid probe to RNA from the subject is indicative of a particular allele complementary to the probe. For representative examples of use of nucleic acid probes, see, for example, U.S. Pat. Nos. 5,288,611 and 4,851,330.

Additionally, or alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P., et al., Bioconjug. Chem. 5:3-7 (1994)). The PNA probe can be designed to specifically hybridize to a molecule in a sample suspected of containing one or more of the marker alleles or haplotypes that are associated with at least one pigmentation trait.

Hybridization of the PNA probe is thus diagnostic for the particular pigmentation traits, and can be used to infer at least one pigmentation in the individual from which the template DNA molecule originates.

In one embodiment of the invention, a test sample containing genomic DNA is collected and the polymerase chain reaction (PCR) is used to amplify a fragment comprising one or more polymorphic markers or haplotypes of the present invention. As described herein, identification of a particular marker allele or haplotype associated with certain pigmentation traits, and thus useful for inferring pigmentation traits, can be accomplished using a variety of methods (e.g., sequence analysis, analysis by restriction digestion, specific hybridization, single stranded conformation polymorphism assays (SSCP), electrophoretic analysis, etc.). In another embodiment, the method of inferring a pigmentation trait is accomplished by expression analysis using quantitative PCR (kinetic thermal cycling). This technique can, for example, utilize commercially available technologies, such as TaqMan® (Applied Biosystems, Foster City, Calif.). The technique can assess the presence of an alteration in the expression or composition of a polypeptide or splicing variant(s) that is encoded by a nucleic acid associated with a pigmentation trait. Further, the expression of the variant(s) can be quantified as physically or functionally different.

In another method of the invention, analysis by restriction digestion can be used to detect a particular allele if the allele results in the creation or elimination of a restriction site relative to a reference sequence. Restriction fragment length polymorphism (RFLP) analysis can be conducted, e.g., as described in Current Protocols in Molecular Biology, supra. The digestion pattern of the relevant DNA fragment indicates the presence or absence of the particular allele in the sample.

Sequence analysis can also be used to detect specific alleles or haplotypes. Therefore, in one embodiment, determination of the presence or absence of a particular marker alleles or particular haplotypes comprises sequence analysis of a test sample of DNA or RNA from a subject or individual, (e.g., a human individual). PCR or other appropriate methods can be used to amplify a portion of a nucleic acid associated with a pigmentation trait or skin cancer, and the presence of a specific allele can then be detected directly by sequencing the polymorphic site (or multiple polymorphic sites in a haplotype) of the genomic DNA in the sample.

Allele-specific oligonucleotides can also be used to detect the presence of a particular allele in a nucleic acid template, through the use of dot-blot hybridization of amplified oligonucleotides with allele-specific oligonucleotide (ASO) probes (see, for example, Saiki, R. et al., Nature, 324:163-166 (1986)). An “allele-specific oligonucleotide” (also referred to herein as an “allele-specific oligonucleotide probe”) is an oligonucleotide of approximately 10-50 base pairs or approximately 15-30 base pairs, that specifically hybridizes to a nucleic acid template, and which contains a specific allele at a polymorphic site (e.g., a marker or haplotype as described herein). An allele-specific oligonucleotide probe that is specific for one or more particular nucleic acids as described herein can be prepared using standard methods (see, e.g., Current Protocols in Molecular Biology, supra). PCR can be used to amplify the desired region. The DNA containing the amplified region can be dot-blotted using standard methods (see, e.g., Current Protocols in Molecular Biology, supra), and the blot can be contacted with the oligonucleotide probe. The presence of specific hybridization of the probe to the amplified region can then be detected. Specific hybridization of an allele-specific oligonucleotide probe to DNA from the subject is indicative of a specific allele at a polymorphic site associated with a pigmentation trait or skin cancer (see, e.g., Gibbs, R. et al., Nucleic Acids Res., 17:2437-2448 (1989) and WO 93/22456).

In one preferred embodiment, a method utilizing a detection oligonucleotide probe comprising a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection. In such an embodiment, the amplified DNA serves as the template for the detection probe and the enhancer probe.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

In another embodiment, arrays of oligonucleotide probes that are complementary to target nucleic acid sequence segments from a subject, can be used to identify particular alleles at polymorphic sites. For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Bier, F. F., et al. Adv Biochem Eng Biotechnol 109:433-53 (2008); Hoheisel, J. D., Nat Rev Genet 7:200-10 (2006); Fan, J. B., et al. Methods Enzymol 410:57-73 (2006); Raqoussis, J. & Elvidge, G., Expert Rev Mol Diagn 6:145-52 (2006); Mockler, T. C., et al Genomics 85:1-15 (2005), and references cited therein, the entire teachings of each of which are incorporated by reference herein). Many additional descriptions of the preparation and use of oligonucleotide arrays for detection of polymorphisms can be found, for example, in U.S. Pat. No. 6,858,394, U.S. Pat. No. 6,429,027, U.S. Pat. No. 5,445,934, U.S. Pat. No. 5,700,637, U.S. Pat. No. 5,744,305, U.S. Pat. No. 5,945,334, U.S. Pat. No. 6,054,270, U.S. Pat. No. 6,300,063, U.S. Pat. No. 6,733,977, U.S. Pat. No. 7,364,858, EP 619 321, and EP 373 203, the entire teachings of which are incorporated by reference herein.

Other methods of nucleic acid analysis that are available to those skilled in the art can be used to detect a particular allele at a polymorphic site. Representative methods include, for example, direct manual sequencing (Church and Gilbert, Proc. Natl. Acad. Sci. USA, 81: 1991-1995 (1988); Sanger, F., et al., Proc. Natl. Acad. Sci. USA, 74:5463-5467 (1977); Beavis, et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V., et al., Proc. Natl. Acad. Sci. USA, 86:232-236 (1989)), mobility shift analysis (Orita, M., et al., Proc. Natl. Acad. Sci. USA, 86:2766-2770 (1989)), restriction enzyme analysis (Flavell, R., et al., Cell, 15:25-41 (1978); Geever, R., et al., Proc. Natl. Acad. Sci. USA, 78:5081-5085 (1981)); heteroduplex analysis; chemical mismatch cleavage (CMC) (Cotton, R., et al., Proc. Natl. Acad. Sci. USA, 85:4397-4401 (1985)); RNase protection assays (Myers, R., et al., Science, 230:1242-1246 (1985); use of polypeptides that recognize nucleotide mismatches, such as E. coli mutS protein; and allele-specific PCR.

Other methods of nucleic acid analysis that are available to those skilled in the art can be used to detect a particular allele at a polymorphic site. Representative methods include, for example, direct manual sequencing (Church and Gilbert, Proc. Natl. Acad. Sci. USA, 81: 1991-1995 (1988); Sanger, F., et al., Proc. Natl. Acad. Sci. USA, 74:5463-5467 (1977); Beavis, et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V., et al., Proc. Natl. Acad. Sci. USA, 86:232-236 (1989)), mobility shift analysis (Orita, M., et al., Proc. Natl. Acad. Sci. USA, 86:2766-2770 (1989)), restriction enzyme analysis (Flavell, R., et al., Cell, 15:25-41 (1978); Geever, R., et al., Proc. Natl. Acad. Sci. USA, 78:5081-5085 (1981)); heteroduplex analysis; chemical mismatch cleavage (CMC) (Cotton, R., et al., Proc. Natl. Acad. Sci. USA, 85:4397-4401 (1985)); RNase protection assays (Myers, R., et al., Science, 230:1242-1246 (1985); use of polypeptides that recognize nucleotide mismatches, such as E. coli mutS protein; and allele-specific PCR.

In another embodiment of the invention, a pigmentation trait of an individual can be inferred or skin cancer susceptibility determined by examining expression and/or composition of a polypeptide encoded by a nucleic acid that is associated with the pigmentation trait or disease in those instances where the genetic marker(s) or haplotype(s) as described herein result in a change in the composition or expression of the polypeptide. In certain embodiments, expression analysis of a gene selected from the group consisting of TYR, TYRP1 and ASIP is performed. In certain other embodiments, expression analysis of a gene selected from the group consisting of MC1R, SLC24A4, KITLG, TYR, OCA2, and TYRP1. The polymorphic markers described herein may also have the biological effect through their influence on the expression of nearby genes, or alternatively by affecting the composition of polypeptides encoded by nearby genes. Thus, it is contemplated that the pigmentation trait or the skin cancer risk can in those instances be inferred by examining expression and/or composition of one of these genes or polypeptides they encode, in those instances where the genetic marker or haplotype of the present invention results in a change in the composition or expression of the polypeptide. Thus, the polymorphic markers of the present invention, and/or haplotypes comprising at least two of those polymorphic markers, that are associated to at least one pigmentation trait or skin cancer may play a role through their effect on one or more of these nearby genes. Possible mechanisms affecting these genes include, e.g., effects on transcription, effects on RNA splicing, alterations in relative amounts of alternative splice forms of mRNA, effects on RNA stability, effects on transport from the nucleus to cytoplasm, and effects on the efficiency and accuracy of translation.

A variety of methods can be used for detecting protein expression levels, including enzyme linked immunosorbent assays (ELISA), Western blots, immunoprecipitations and immunofluorescence. A test sample from a subject that includes the protein is assessed for the presence of an alteration in the expression and/or an alteration in composition of the polypeptide. The test sample may be any sample that contains detectable amounts of the polypeptide. In certain embodiments, the test sample is a sample that contains protein from at least one specific tissue. The specific tissue can be a tissue characteristic of a particular pigmentation trait and/or skin cancer, including but not limited to, hair samples, hair follicles, eye fluid (e.g., intraocular fluid or aqueous humor) or skin cells including skin epidermal cells, skin dermal cells. An alteration in expression of a polypeptide encoded by a nucleic acid associated with the at least one pigmentation trait can be, for example, an alteration in the quantitative polypeptide expression (i.e., the amount of polypeptide produced). An alteration in the composition of a polypeptide can be an alteration in the qualitative polypeptide expression (e.g., expression of a mutant polypeptide or of a different splicing variant). As a consequence, in one embodiment, pigmentation traits or skin cancer risk can be inferred by detecting the expression of, or by detecting a particular splicing variant encoded by a nucleic acid that is associated with the pigmentation trait or the skin cancer. In another embodiment, a particular pattern of splicing variants is determined, such as a for example the ratio of expression of one splicing variant to the expression of another splicing variant.

Both such alterations (quantitative and qualitative) can also be present. An “alteration” in the polypeptide expression or composition, as used herein, refers to an alteration in expression or composition in a test sample, as compared to the expression or composition of the polypeptide in a control sample. A control sample is a sample that corresponds to the test sample (e.g., is from the same type of cells), and is from a subject who does not have the particular pigmentation trait. Alternatively, the control sample is a sample from a subject, or from a group of subjects, from the general population. In such cases the control sample represents the general population, which includes individuals with the particular pigmentation trait or skin cancer. In one embodiment, the control sample is from a subject that does not possess a risk marker allele or haplotype as described herein. Similarly, the presence of one or more different splicing variants in the test sample, or the presence of significantly different amounts of different splicing variants in the test sample, as compared with the control sample, can be indicative of the particular pigmentation trait or several pigmentation traits, or the skin cancer, and can therefore be used to infer the pigmentation trait or several pigmentation traits, or predict susceptibility of the skin cancer. An alteration in the expression or composition of the polypeptide in the test sample, as compared with the control sample, can be indicative of a specific allele in the instance where the allele alters a splice site relative to the reference in the control sample. Various means of examining expression or composition of a polypeptide encoded by a nucleic acid are known to the person skilled in the art and can be used, including spectroscopy, colorimetry, electrophoresis, isoelectric focusing, and immunoassays (e.g., David et al., U.S. Pat. No. 4,376,110) such as immunoblotting (see, e.g., Current Protocols in Molecular Biology, particularly chapter 10, supra).

For example, in one embodiment, an antibody (e.g., an antibody with a detectable label) that is capable of binding to a polypeptide encoded by a nucleic acid associated with at least one pigmentation trait can be used. Antibodies can be polyclonal or monoclonal. An intact antibody, or a fragment thereof (e.g., Fv, Fab, Fab′, F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a labeled secondary antibody (e.g., a fluorescently-labeled secondary antibody) and end-labeling of a DNA probe with biotin such that it can be detected with fluorescently-labeled streptavidin.

In one embodiment of this method, the level or amount of polypeptide encoded by a nucleic acid associated with at least one pigmentation trait in a test sample is compared with the level or amount of the polypeptide in a control sample. A level or amount of the polypeptide in the test sample that is higher or lower than the level or amount of the polypeptide in the control sample, such that the difference is statistically significant, is indicative of an alteration in the expression of the polypeptide encoded by the nucleic acid, and is diagnostic for a particular allele or haplotype responsible for causing the difference in expression. Alternatively, the composition of the polypeptide in a test sample is compared with the composition of the polypeptide in a control sample. In another embodiment, both the level or amount and the composition of the polypeptide can be assessed in the test sample and in the control sample.

In another embodiment, at least one pigmentation trait is inferred, or association to at least one pigmentation trait or skin cancer is determined, by detecting at least one marker or haplotypes as described herein, in combination with an additional protein-based, RNA-based or DNA-based assay. The methods of the invention can also be used in combination with information about family history and/or racial background.

Kits

Kits useful in the methods of the invention comprise components useful in any of the methods described herein for inferring a pigmentation trait or for diagnosing susceptibility to skin cancer (e.g., melanoma). This includes for example kits that include reagents for the determination of the presence or absence of at least one allele of at least one polymorphic marker, wherein the presence or the absence of the at least one allele is indicative of at least one pigmentation trait or skin cancer, or can be used for inferring at least one pigmentation trait. Kits of the invention can also include reagents for determination of protein expression levels, presence and/or absence of splicing variants, or reagents useful in other methods as described herein.

The kits of the invention can include for example, hybridization probes, restriction enzymes (e.g., for RFLP analysis), allele-specific oligonucleotides, antibodies that bind to an altered polypeptide encoded by a nucleic acid of the invention as described herein (e.g., a genomic segment comprising at least one polymorphic marker and/or haplotype of the present invention) or to a non-altered (native) polypeptide encoded by a nucleic acid of the invention as described herein, means for amplification of a segment of a nucleic acid sample that includes a nucleic acid associated with at least one pigmentation trait, means for analyzing the nucleic acid sequence of a sample comprising a nucleic acid associated with at least one pigmentation trait, means for analyzing the amino acid sequence of a polypeptide encoded by a nucleic acid associated with at least one pigmentation trait, etc. The kits can for example include necessary buffers, nucleic acid primers for amplifying nucleic acids of the invention (e.g., one or more of the polymorphic markers as described herein), and reagents for allele-specific detection of the fragments amplified using such primers and necessary enzymes (e.g., DNA polymerase). The kits can additionally provide reagents for assays to be used in combination with the methods of the present invention, e.g., reagents for assays to be assessed in combination with the diagnostic assays described herein.

In one embodiment, the invention is a kit for assaying a sample from a subject to infer at least one pigmentation trait in a subject, or determine a susceptibility to a skin cancer in a subject, wherein the kit comprises reagents necessary for selectively detecting at least one allele of at least one polymorphism as described herein. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism, wherein the polymorphism is selected from the group consisting of the polymorphisms as listed in Table 10 and 21 and polymorphic markers in linkage disequilibrium therewith (e.g., the polymorphic markers listed in Table 11, 14, 25 and 26). In certain embodiments, the kit comprises reagents for detecting at least one marker selected from rs1015362, rs4911414, rs1126809 and rs1408799. In one embodiment the fragment is at least 20 base pairs in size. Such oligonucleotides or nucleic acids (e.g., oligonucleotide primers) can be designed using portions of the nucleic acid sequence flanking polymorphisms (e.g., SNPs or microsatellites) that are associated with at least one pigmentation trait, as described herein. In another embodiment, the kit comprises one or more labeled nucleic acids capable of allele-specific detection of one or more specific polymorphic markers or haplotypes associated with at least one pigmentation trait, and reagents for detection of the label. Suitable labels include, e.g., a radioisotope, a fluorescent label, an enzyme label, an enzyme co-factor label, a magnetic label, a spin label, an epitope label.

In particular embodiments, the polymorphic marker or haplotype to be detected by the reagents of the kit comprises one or more markers, two or more markers, three or more markers, four or more markers or five or more markers selected from the group consisting of the markers in Table 11, 25 and 26. In another embodiment, the marker or haplotype to be detected comprises the markers listed in Table 10 and Table 21. In another embodiment, the marker or haplotype to be detected comprises at least one marker from the group of markers in strong linkage disequilibrium, as defined by values of r² greater than 0.2, to at least one of the group of markers consisting of the markers listed in Table 10 and Table 21. In yet another embodiment, the marker or haplotype to be detected comprises at least one marker selected from the group of markers listed in Table A. In another embodiment, the marker or haplotype to be detected is selected from the group of markers listed in Table A, and markers in linkage disequilibrium therewith. In certain embodiments, linkage disequilibrium therewith indicates a value for the measure r² of at least 0.2. In other embodiments, linkage disequilibrium is determined for the CEU population of HapMap samples (http://www.hapmap.org).

Nucleic Acids and Polypeptides

The nucleic acids and polypeptides described herein can be used in methods and kits of the present invention, as described in the above.

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb, 0.3kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention.

An isolated nucleic acid molecule or nucleotide sequence can include a nucleic acid molecule or nucleotide sequence that is synthesized chemically or by recombinant means. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20).

Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988).

In another embodiment, the percent identity between two amino acid sequences can be accomplished using the GAP program in the GCG software package (Accelrys, Cambridge, UK).

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques well known to the skilled person. The amplified DNA can be labeled (e.g., radiolabeled) and used as a probe for screening a cDNA library derived from human cells. The cDNA can be derived from mRNA and contained in a suitable vector. Corresponding clones can be isolated, DNA can obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

In general, the isolated nucleic acid sequences of the invention can be used as molecular weight markers on Southern gels, and as chromosome markers that are labeled to map related gene positions. The nucleic acid sequences can also be used to compare with endogenous DNA sequences from individuals to identify a particular pigmentation trait, or determine susceptibility to a skin cancer, and as probes, such as to hybridize and discover related DNA sequences or to subtract out known sequences from a sample (e.g., subtractive hybridization). The nucleic acid sequences can further be used to derive primers for genetic fingerprinting, to raise anti-polypeptide antibodies using immunization techniques, and/or as an antigen to raise anti-DNA antibodies or elicit immune responses.

Antibodies

Polyclonal antibodies and/or monoclonal antibodies that specifically bind one form of the gene product (e.g., polypeptide) but not to the other form of the gene product are also provided. Antibodies are also provided which bind a portion of either the variant or the reference gene product that contains the polymorphic site or sites. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies may also be useful for assessing expression of variant proteins in individuals or groups of individuals characterized by a certain pigmentation pattern that is associated with the presence of the variant proteins, or for determining suscepbility to skin cancer in individuals. Antibodies specific for a variant protein of the present invention that is encoded by a nucleic acid that comprises at least one polymorphic marker or haplotype as described herein can be used to screen for the presence of the variant protein, for example to screen a protein sample to infer a certain pigmentation trait, as indicated by the presence of the variant protein.

Antibodies can be used in other methods. Thus, antibodies are useful as diagnostic tools for evaluating proteins, such as variant proteins of the invention, in conjunction with analysis by electrophoretic mobility, isoelectric point, tryptic or other protease digest, or for use in other physical assays known to those skilled in the art. Antibodies may also be used in tissue typing. In one such embodiment, a specific variant protein has been correlated with expression in a specific tissue type, and antibodies specific for the variant protein can then be used to identify the specific tissue type.

The present invention further relates to kits for using antibodies in the methods described herein. This includes, but is not limited to, kits for detecting the presence of a variant protein in a test sample. One preferred embodiment comprises antibodies such as a labeled or labelable antibody and a compound or agent for detecting variant proteins in a biological sample, means for determining the amount or the presence and/or absence of variant protein in the sample, and means for comparing the amount of variant protein in the sample with a standard, as well as instructions for use of the kit.

The skilled person will appreciate that the foregoing discussion of the methods, nucleic acids, polypeptides, antibodies, apparatus and kits of the present invention for relate equally to embodiments for inferring at least one pigmentation trait and embodiments that relate to a susceptibility to disease, e.g., skin cancer (e.g., melanoma) in an individual.

Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

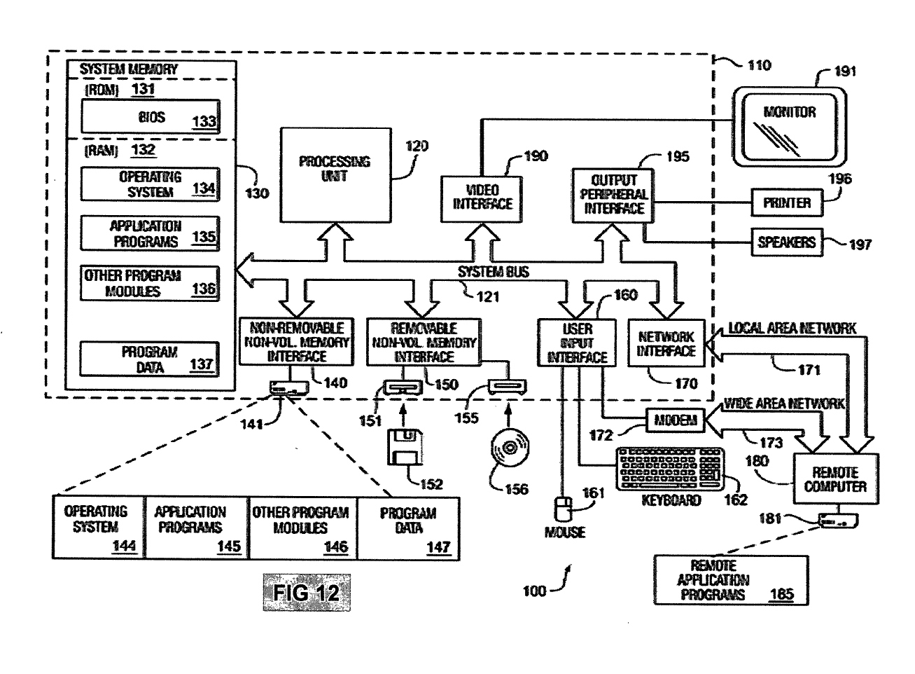

FIG. 12 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method or apparatus of the claims. Neither should the computing environment 100 be interpreted as having any dependency or requirement relating to any one or combination of components illustrated in the exemplary operating environment 100.

The steps of the claimed method and system are operational with numerous other general purpose or special purpose computing system environments or configurations. Examples of well known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

With reference to FIG. 12, an exemplary system for implementing the steps of the claimed method and system includes a general purpose computing device in the form of a computer 110. Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other) data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 12 illustrates operating system 134, application programs 135, other program modules 136, and program data 137.

The computer 110 may also include other removable/non-removable, volatile/nonvolatile computer storage media. By way of example only, FIG. 12 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 12, provide storage of computer readable instructions, data structures, program modules and other data for the computer 110. In FIG. 12, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 12. The logical connections depicted in FIG. 12 include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 12 illustrates remote application programs 185 as residing on memory device 181. It will be appreciated that the network connections shown are exemplary and other means of establishing a communications link between the computers may be used.

Although the forgoing text sets forth a detailed description of numerous different embodiments of the invention, it should be understood that the scope of the invention is defined by the words of the claims set forth at the end of this patent. The detailed description is to be construed as exemplary only and does not describe every possibly embodiment of the invention because describing every possible embodiment would be impractical, if not impossible. Numerous alternative embodiments could be implemented, using either current technology or technology developed after the filing date of this patent, which would still fall within the scope of the claims defining the invention.

While the risk evaluation system and method, and other elements, have been described as preferably being implemented in software, they may be implemented in hardware, firmware, etc., and may be implemented by any other processor. Thus, the elements described herein may be implemented in a standard multi-purpose CPU or on specifically designed hardware or firmware such as an application-specific integrated circuit (ASIC) or other hard-wired device as desired, including, but not limited to, the computer 110 of FIG. 12. When implemented in software, the software routine may be stored in any computer readable memory such as on a magnetic disk, a laser disk, or other storage medium, in a RAM or ROM of a computer or processor, in any database, etc. Likewise, this software may be delivered to a user or a diagnostic system via any known or desired delivery method including, for example, on a computer readable disk or other transportable computer storage mechanism or over a communication channel such as a telephone line, the internet, wireless communication, etc. (which are viewed as being the same as or interchangeable with providing such software via a transportable storage medium).

Thus, many modifications and variations may be made in the techniques and structures described and illustrated herein without departing from the spirit and scope of the present invention. Thus, it should be understood that the methods and apparatus described herein are illustrative only and are not limiting upon the scope of the invention.

Accordingly, the invention relates to computer-implemented applications using the polymorphic markers and haplotypes described herein, and genotype and/or disease/trait-association data derived therefrom. This includes association data with skin cancers and data associating particular markers and/or haplotypes with certain pigmentation traits, as described herein. Such applications can be useful for storing, manipulating or otherwise analyzing genotype data that is useful in the methods of the invention. One example pertains to storing genotype information derived from an individual on readable media, so as to be able to provide the genotype information to a third party (e.g., the individual, a guardian of the individual, a health care provider or genetic analysis service provider), or for deriving information from the genotype data, e.g., by comparing the genotype data to information about genetic risk factors contributing to increased susceptibility to the skin disease or pigmentation trait, and reporting results based on such comparison.

In general terms, computer-readable media has capabilities of storing (i) identifer information for at least one polymorphic marker or a haplotype, as described herein; (ii) an indicator of the frequency of at least one allele of said at least one marker, or the frequency of a haplotype, in individuals with the skin cancer, or the particular pigmentation trait; and an indicator of the frequency of at least one allele of said at least one marker, or the frequency of a haplotype, in a reference population. The reference population can be a disease-free population of individuals. Alternatively, the reference population is a random sample from the general population, and is thus representative of the population at large. The frequency indicator may be a calculated frequency, a count of alleles and/or haplotype copies, or normalized or otherwise manipulated values of the actual frequencies that are suitable for the particular medium.

The markers and haplotypes described herein to be associated with increased susceptibility (e.g., increased risk) of the skin cancer or the pigmentation trait, are in certain embodiments useful for interpretation and/or analysis of genotype data. Thus in certain embodiments, an identification of an at-risk allele for the skin cancer or pigmentation trait, as shown herein, or an allele at a polymorphic marker in LD with any one of the markers shown herein to be associated with the skin cancer or the pigmentation trait, is indicative of the individual from whom the genotype data originates is at increased risk of the particular cancer or trait. In one such embodiment, genotype data is generated for at least one particular polymorphic marker, or a marker in linkage disequilibrium therewith. The genotype data is subsequently made available to a third party, such as the individual from whom the data originates, his/her guardian or representative, a physician or health care worker, genetic counselor, or insurance agent, for example via a user interface accessable over the internet, together with an interpretation of the genotype data, e.g., in the form of a risk measure (such as an absolute risk (AR), risk ratio (RR) or odds ratio (OR)) for the disease. In another embodiment, at-risk markers identified in a genotype dataset derived from an individual are assessed and results from the assessment of the risk conferred by the presence of such at-risk varians in the dataset are made available to the third party, for example via a secure web interface, or by other communication means. The results of such risk assessment can be reported in numeric form (e.g., by risk values, such as absolute risk, relative risk, and/or an odds ratio, or by a percentage increase in risk compared with a reference), by graphical means, or by other means suitable to illustrate the risk to the individual from whom the genotype data is derived.

The present invention will now be further illustrated by the following non-limiting Examples.

### Examples

### Example 1

### Variants Associated with Hair, Eye and Skin Pigmentation

A genome-wide association scan for sequence variants influencing hair color, eye color, freckles and skin sensitivity to sun was performed, using a set of 317 thousand SNPs genotyped in 2,986 Icelanders. Promising SNPs were tested in replication samples from 2,718 Icelanders and 1,214 Dutch individuals.

Methods

Icelandic Samples

A total of 2,986 Icelandic adults, recruited through cardiovascular, neoplastic, neurologic and metabolic study projects, were genotyped for 317,000 SNPs using the HumanHap300 BeadChip (Illumina, San Diego, Calif., USA). These studies were approved by the Data Protection Commission of Iceland and the National Bioethics Committee of Iceland. Written informed consent was obtained from all participants. Personal identifiers associated with phenotypic information and blood samples were encrypted using a third-party encryption system as previously described (Gulcher, J. R., et al., Eur J Hum Genet 8, 739-42 (2000)). Only individuals with a genotype yield over 98% were included in the study. A second sample of 2,714 Icelandic individuals was recruited in a similar fashion and genotyped to replicate the SNPs identified in the genome-wide scan.

Each participant completed a questionnaire that included questions about natural eye color categories (blue/grey, green, black/brown), natural hair color categories (red/reddish, blond, dark blond/light brown, brown/black) and the presence of freckles at any time (Table 1). Skin sensitivity to sun was self-assessed using the Fitzpatrick skin-type score (Fitzpatrick, T. B. Arch Dermatol 124, 869-71 (1988)), where the lowest score (I) represents very fair skin that is very sensitive to UVR and the highest score (IV) represents dark skin that tans rather than burns in reaction to UVR exposure. Individuals scoring I and II were classified as being sensitive to sun and individuals scoring III and IV were classified as not being sensitive to sun.

No objective measurements of pigmentation, e.g. spectrophotometry, were performed. The benefits of the self-reported measurements are that they are cheap and easy to collect, but their subjective nature is likely to introduce misclassifications leading to loss of power in the discovery phase and a decrease of prediction accuracy.

Dutch Samples

The most significantly associated SNPs identified in the genome-wide scans performed on the Icelandic discovery sample were genotyped and tested for association in a sample of 1,214 Dutch individuals. The Dutch sample was composed of 705 males recruited for a prostate cancer study (Gudmundsson, J. et al. Nat Genet 39, 631-7 (2007)) and 518 females recruited for a breast cancer study by the Radboud University Nijmegen Medical Centre (RUNMC) and through a population-based cancer registry held by the Comprehensive Cancer Centre IKO in Nijmegen. All individuals were of self-reported European ancestry. The study protocol was approved by the Institutional Review Board of Radboud University and all study subjects gave written informed consent for the collection of questionnaire data on lifestyle, medical history, and family history.

As in the case of the Icelandic samples, information about pigmentation traits for the Dutch sample was obtained through a questionnaire. The questions about natural eye and hair color were the same as those in the Icelandic questionnaire, with the addition of a category of “other” eye color. A total of 5.9% of the Dutch participants selected this category and were excluded from our analysis. Skin sensitivity to sun was assessed by two questions about the tendency of individuals to burn or tan when exposed to sun without sun block protection. The answers to these two questions were used to create a dichotomized grouping of individuals according to sensitivity to sun, corresponding to the grouping used for the Icelandic sample. Two questions from the Dutch questionnaire assessed the density of freckles on the face and arms, respectively. For the sake of comparison with the Icelandic data, participants reporting freckles at either location were considered as having freckles present, whereas those reporting absence of freckles at both locations were considered to have no freckles. In addition, the Dutch questionnaire included questions about skin color category (white, white with brownish tint and light-brown), the number of naevi on the left fore arm and the lifetime number of serious sunburns.

Statistical Methods

In the genome-wide association stage Icelandic case- and control-samples were assayed with the Infinium HumanHap300 SNP chips (Illumina, SanDiego, Calif., USA), containing 317,511 SNPs, out of which 316,515 were polymorphic and satisfied our quality criteria.

A likelihood procedure described in a previous publication (Gretarsdottir, S. et al. Nat Genet 35, 131-8 (2003)) was used for the association analyses. Allele-specific OR was calculated assuming a multiplicative model (Falk, C. T. & Rubinstein, P. Ann Hum Genet 51 (Pt 3), 227-33 (1987)). Results from multiple case-control groups were combined using a Mantel-Haenszel model (Mantel, N. & Haenszel, W. J Natl Cancer Inst. 22, 719-48 (1959)). In Tables 2-4, P values for variants at MC1R, TYR and OCA2 were calculated by conditioning on the effect of the other variant at that locus.

Pigmentation Prediction

A model to predict eye and hair pigmentation was created based on the Icelandic discovery sample (FIG. 2). A generalized linear model, where eye color was treated as a categorical response with three categories and genotypes at all associated sequence variants were used as covariates, was used to model eye color. A two step model was employed for the prediction of hair color. The first step involved predicting red hair and was based solely on the MC1R coding variants. The second step involved modeling non-red hair color as an ordinal variable with dark-blond or light-brown hair being between the extremes of blond and brown or black hair. Eye and hair pigmentation in the Icelandic and Dutch replication samples were then predicted based on the model parameters estimated in the Icelandic discovery sample.

Correction for Relatedness and Genomic Control

Some of the individuals in the Icelandic case-control groups were related to each other, causing the aforementioned chi-square test statistic to have a mean>1 and median>0.675². We estimated the inflation factor by using a previously described procedure where we simulated genotypes through the genealogy of 731,175 Icelanders (Grant, S. F. et al. Nat Genet 38, 320-3 (2006)). For the initial discovery samples, where genotypes for the 316,515 genome-wide scan SNPs were available, we also estimated the inflation factor by using genomic controls and calculating the average of the 316,515 chi-square statistics, and by computing the median of the 316,515 chi-square statistics and dividing it by 0.675² as previously described (Devlin, B. & Roeder, K. Biometrics 55, 997-1004 (1999); Devlin, B. et al. Nature Genetics 36, 1129-1130 (2004)).

Single SNP Genotyping

SNP genotyping was carried out by the Centaurus (Nanogen) platform (Kutyavin, I. V. et al. Nucleic Acids Research 34, e128 (2006)). The quality of each Centaurus SNP assay was evaluated by genotyping each assay in the CEU and/or YRI HapMap samples and comparing the results with the HapMap data. Assays with >1.5% mismatch rate were not used and a linkage disequilibrium (LD) test was used for markers known to be in LD.

Controlling for Population Stratification

Most of the variants showing significant association to pigmentation are also present in frequencies that differ among European populations and between European, Asian and African populations. These frequency differences are to be expected given the difference in pigmentation between the populations. However, if our method of discovery would have been applied to a stratified sample of Europeans, without taking this stratification into account, then variants with population frequencies correlating with pigmentation could show spurious association to pigmentation. We therefore performed a series of tests to search for signs of stratification even though the Icelandic population has been relatively isolated throughout its history.

First, we applied to the analysis the method of genomic control, which takes into account the genome-wide inflation of the chi-square statistics. The inflation factors we observed were similar to inflation factors estimated from known relationships between individuals, suggesting the overall inflation due to stratification is small.

Second, from a published set of 400 SNPs, known to have differing frequencies between European populations (Seldin, M. F. et al. PLoS Genet 2, e143 (2006)), we selected a subset of 97 SNPs also present on the Illumina 317K Human Hap chip. We then tested for LD between 4,417 pairs of markers on different chromosomes among 1,984 Icelanders unrelated at a meiotic distance of 3. Out of the 4,417 pairs tested, 225 had P<0.05 compared to 220.8 expected and 6 had P<0.001 compared to 4.4 expected. We also tested for LD between the 97 SNPs and the 9 SNPs, resulting in 834 tests where the two markers were not on the same chromosome. Again we observed no significant excess of low P values (observed 39 compared to 41 expected at P<0.05 and observed 2 compared to 0.8 expected at P<0.001).

Third, the gene encoding lactase is well described and has a very large degree of variation between populations (Bersaglieri, T. et al. Am J Hum Genet 74, 1111-20 (2004)), but no known association to pigmentation. We chose the intra-genic marker rs2322659, and tested its LD with the 9 SNPs associating with pigmentation (P>0.01 in all instances). We also performed the 6 tests for association of rs2322659 to pigmentation without detecting any significant association.

Finally, we applied the EIGENSTRAT method (Price, A. L. et al. Nat Genet 38, 904-9 (2006)), which relies on patterns of correlation between individuals to detect stratification, to our Icelandic discovery sample. No evidence of substantial stratification was detected, with the largest principal component estimated to explain 0.2% of the overall variation of the data. The correction factors based on correcting for the 10 largest principal components are close to 1 and do not have any impact on our conclusions. Inspection of the first few principal components suggests they correspond to sets of few close relatives, whose relation had not been properly accounted for.

Assessing Signals of Positive Selection

Evidence for the impact of positive selection on SNPs associated to pigmentation traits was examined by applying two different methods to data from the HapMap project (release 21) (Nature 437, 1299-320 (2005)). First, we examined whether the degree of population divergence in allele frequencies among the HapMap groups exceeded expectations based on neutral evolution. Under neutrality, the frequencies of any particular allele in a set of populations are shaped by the counteracting forces of genetic drift, gene flow and mutation, which constrain the expected range of allele frequencies differences expected between the populations. When the observed divergence between populations is in the upper extreme of the expected range, or outside it, the neutral model may be rejected in favor of one in which allele frequencies have been shaped by population differences in the intensity selective forces (Beaumont, M. A. & Nichols, R. A. Proceedings of the Royal Society of London Series B-Biological Sciences 263, 1619-1626 (1996)).

The Wahlund F_{ST} statistic,

$${F}_{ST} = \frac{var ( p )}{\overline{p} ( 1 - \overline{p} )} ,$$

was used to measure allele frequency differences between populations, where var(p) represents the variance of the frequencies of an allele from a bi-allelic SNP, and p̅ represents the average frequency of the allele, among the populations under consideration. This statistic was calculated for all HapMap SNPs genotyped in at least two HapMap samples, with 3,020,798 SNPs yielding F_{ST} values based on all three HapMap samples (CEU, YRI and ASN), and 3,064,337, 3,118,875 and 3,094,443 for the population pairs CEU-YRI, CEU-ASN and YRI-ASN, respectively. For each combination of HapMap samples, the SNPs were grouped into 50 bins according to the overall frequency of the more common allele and using an interval of 0.01. To assess whether a particular SNP showed an unusually degree of population divergence, the percentile rank of each SNP's F_{ST} value was determined within each bin for each combination of HapMap samples.

The second method used to detect signals of positive selection is based on examining the pattern of diversity within populations. Under neutrality, there is an expected positive relationship between the frequency of an allele, its age, the variability at linked sites and the extent to which linkage disequilibrium (LD) with other loci decays at increasing physical distance. Common alleles with unusually low diversity at linked sites and/or slow decay of LD with increasing physical distance represent likely targets of recent positive selection. We used the relative extended haplotype homozygosity (rEHH) to assess the fragmentation of haplotypes around putative selected variants (Sabeti, P. C. et al. Nature 419, 832-7 (2002)). To simplify comparisons between different genomic regions, we calculated a single integrated rEHH (irEHH) value for each allele, representing the area beneath the line defined by the rEHH point estimates that are obtained as haplotypes are extended in both directions from the allele being tested (until the EHH value in both directions has fallen below 0.05) (Helgason, A. et al. Nat Genet 39, 218-225 (2007); Voight, B. F., et al. PLoS Biol 4, e72 (2006)). Calculations were performed for all HapMap SNPs in the CEU HapMap sample with a minor allele frequency>1%, yielding irEHH values for a total number of 4,906,866 alleles. To make comparisons of irEHH values meaningful between regions with different rates of recombination, the positions of SNPs were defined in cM for these calculations (using recombination rate maps for phase II of the HapMap, which are available at the HapMap website). To determine whether a particular irEHH value could be considered as unusually great, thereby indicating the action of positive selection, we grouped all HapMap SNPs of the same frequency in the CEU HapMap group into separate bins and calculated the percentile rank of each irEHH value within each of the bins.

Results

The frequencies of natural hair and eye color categories, skin sensitivity categories and presence of freckles in the two Icelandic samples and the Dutch sample are shown by sex in Table 1. The samples are broadly similar, although the Icelanders more often have red hair, freckles, and green eyes, but less often brown eyes. The most striking difference between the sexes is the higher frequency of green eyes and freckles in females. The higher frequency of green eyes in females is consistent with a previous report where eye color was assessed by a single expert (Sturm, R. A. & Frudakis, T. N. Trends Genet 20, 327-32 (2004); Frudakis, T. et al. Genetics 165, 2071-83 (2003); Duffy, D. L. et al. Am J Hum Genet 80, 241-52 (2007)).

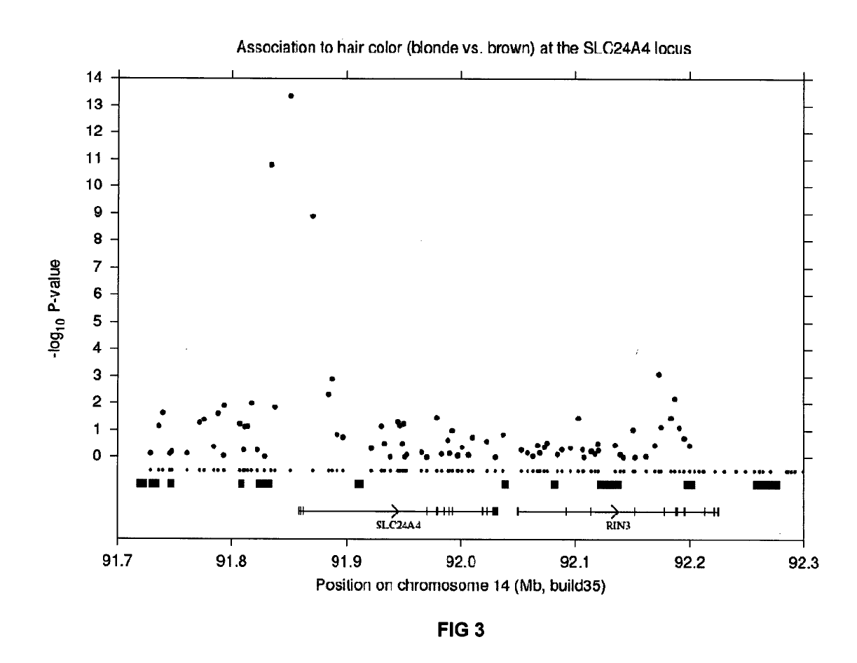

The association of sequence variants to pigmentation traits was examined in six genome-wide association scans of the Icelandic discovery sample. Two scans were performed for eye color (blue vs. green and blue vs. brown), two scans were performed for hair color (red vs. non-red and blond vs. brown), and two for skin pigment traits (skin sensitivity to sun and presence of freckles). Overall, these genome scans revealed 104 association signals that reached genome-wide significance (P<1.5×10⁻⁷), accounted for by 60 distinct SNPs (Table 5), of which 32 showed genome-wide association to only one pigmentation trait, 12 to two traits and 16 to three traits. The 60 SNPs were clustered in five different genomic regions on five different chromosomes (6, 12, 14, 15 and 16, FIGS. 3-7), with the largest covering 1 MB on chromosome 16 and the smallest amounting to a single SNP on chromosome 12. Notably, two of the regions overlap with or are nearby well-known pigmentation genes (MC1R on chromosome 16 and OCA2 on chromosome 15) and one of the regions is near a strong candidate pigmentation gene (KITLG on chromosome 12). One of the remaining two regions overlaps with the SLC24A4 on chromosome 14 that belongs to the same family as SLC24A5, a recently discovered pigmentation gene (Lamason, R. L. et al. Science 310, 1782-6 (2005)). The other is located between the genes IRF4 and SEC5L1 on chromosome 6, neither of which have been reported previously to affect pigmentation.

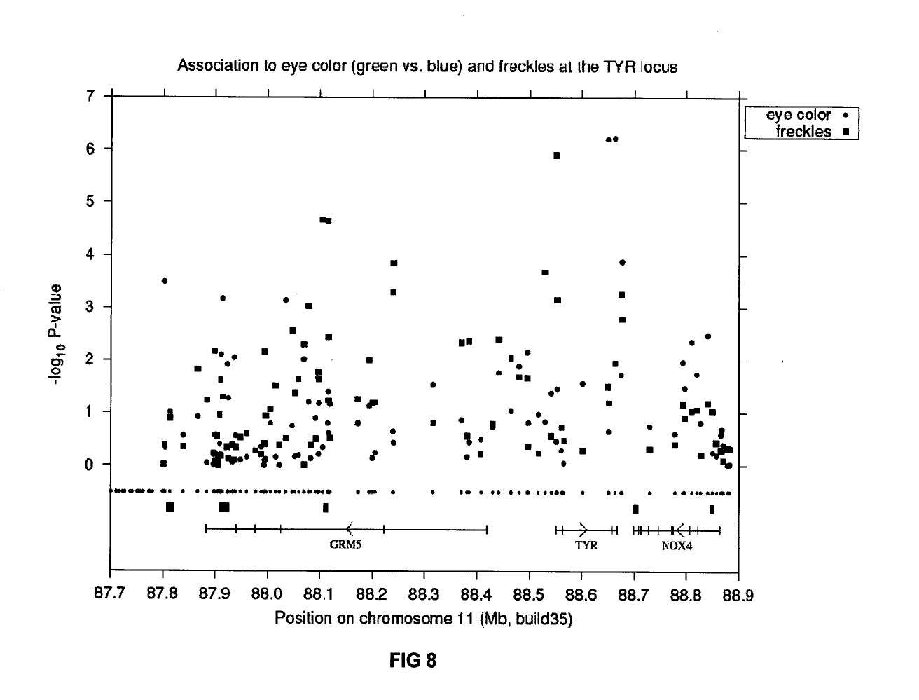

We defined a subset of seven SNPs that capture the strongest association signals within these five regions based on the Icelandic discovery sample. In addition, we chose two SNPs located in TYR, a key pigmentation gene on chromosome 11, that showed suggestive association in two of the scans (P<6×10⁻⁶, FIG. 8). No SNPs in other candidate genes remained significant after correcting for the number of SNPs in these candidate genes, possibly due to lack of power. All nine SNPs were significantly associated to the same pigmentation traits in the Icelandic and Dutch replication samples (Tables 2-4 and 6). All nine SNPs were significant in the combined discovery and replication samples, after correcting for the 317,000 SNPs tested and the 6 genome-wide scans performed (P<2.6×10⁻⁸). We summarize primary and secondary pigmentation trait associated to the SNPs in these 6 genomic regions (FIG. 1) in separate sections and discuss whether they have been subject to positive selection (Table 7).

MC1R Region

A total of 38 SNPs spanning a 1 Mb region of strong LD on chromosome 16 show genome-wide significant association to red hair, skin sensitivity to sun and freckles, and a suggestive association to blond hair. The SNP rs4785763 most effectively capture the association (OR=5.62, P=3.2×10⁻⁵⁶ red hair, OR=2.03, P=1.2×10⁻³³ freckles). This region contains the well-documented melanocortin 1 receptor (MC1R) gene. Over 30 non-synonymous mutations have been described in populations of European ancestry that impair the normal function of the MC1R gene product (Rees, J. L. Am J Hum Genet 75, 739-51 (2004); Makova, K. & Norton, H. Peptides 26, 1901-8 (2005)), leading to the generation of melanosomes containing the red-yellow pheomelanin rather than the brown-black eumelanin (Sturm, R. A., et al. Bioessays 20, 712-21 (1998); Lin, J. Y. & Fisher, D. E. Nature 445, 843-50 (2007)), and resulting in pigmentation traits such as red and blond hair, freckles, fair skin and sensitivity to UVR (Valverde, P., et al. Nat Genet 11, 328-30 (1995); Rees, J. L. Am J Hum Genet 75, 739-51 (2004)). Two non-synonymous MC1R mutations are common enough in European populations to have a major effect on normal differences in pigmentation: R151C (rs1805007) and R160W (rs1805008) (Makova, K. & Norton, H. Peptides 26, 1901-8 (2005)), neither of which is assayed on the Illumina 317K SNP chip. After genotyping these SNPs in the Icelandic and Dutch samples, we found that their T alleles (i.e. the mutated alleles) are correlated with the A allele of rs4785763 and that the strong association of rs4785763 disappeared in both samples when adjusted for rs1805007 and rs1805008. We therefore conclude that the association signal detected in the genome scan is likely accounted for by the previously documented non-synonymous mutations in MC1R. However, as shown herein, the MC1R variants we have discovered may be utilized in combination with other variants described herein for inferring certain pigmentation traits.

The T alleles of rs1805007 and rs1805008 are found at a frequency of 0.142 and 0.108, respectively, in the CEPH Utah (CEU) HapMap sample, but are not present in the East Asian (ASN) and Nigerian Yoruban (YRI) HapMap samples (Nature 437, 1299-320 (2005)). Although this represents only a moderate level of population divergence and is not consistent with the action of a strong selective sweep on these variants in European populations, we note that only 5.13% of HapMap SNPs with the same overall frequency in the CEU and ASN samples show a greater difference between these populations. Moreover, only 6.6% and 6.2% of equally frequent alleles in the CEU sample exhibited greater extended haplotype homozygosity (based on the irEHH statistic) than rs1805007 T and rs1805008 T, respectively. These results suggest that both mutated alleles may have been at least weakly affected by recent positive selection.

Chromosome 6p25.3 Region

Two SNPs that lie only 8 kb apart in region 6p25.3, rs4959270 and rs1540771, show genome-wide significant association to the presence of freckles in the Icelandic sample (Table 5). This small segment lies between two genes, SEC5L1 and IRF4, neither of which is an obvious pigmentation candidate gene; no such genes are found within LD range of the two SNPs. Although strongly correlated (r²=0.77), the A allele of rs1540771 presented the stronger association (OR=1.40, P=1.9×10⁻⁹) and remained significant after adjusting for rs4959270 (P=0.043) while the reverse was not true (P=0.34). The association of rs1540771 to freckles was confirmed in the Icelandic and Dutch replication samples (Table 4). Interestingly, the A allele of rs1540771 shows secondary associations to brown (rather than blond) hair and to skin that is sensitive to UVR (Tables 3 and 4 and FIG. 1). Thus, like MC1R, the variant on 6p25.3 associated to freckles is also associated to sun sensitivity, but unlike MC1R, there is no association to red hair.

The frequency of rs1540771 A is approximately 50% in European populations, but 30% and 5% in the East Asian and YRI HapMap samples, respectively (6.3% of HapMap SNPs of a similar frequency in the CEU and YRI HapMap samples differ more in frequency) and only 4.1% of alleles at the same frequency in the CEU HapMap data set have greater irEHH values. This suggests that rs1540771 A has been subject to positive selection in European populations, perhaps due to its impact on reduced skin pigmentation. In addition SNPs in the neighborhood of rs1540771 were recently shown to be among the SNPs with the strongest longitudinal geographic trend in the British population (Nature 447, 661-78 (2007)).

Tyrosinase Region

The two SNPs chosen for genotyping in the TYR gene, rs1042602 and rs1393350, are found in the same LD block (r²=0.16 in the Icelandic sample), but their effects in association to pigmentation traits are essentially independent. The association of rs1042602 (a non-synonymous S192Y mutation) to freckles was suggestive in the Icelandic discovery sample (OR=1.32, P=5.3×10⁻⁶) and was confirmed in the replication samples (combined P=1.5×10⁻¹¹, Table 4). Although previous studies have reported suggestive associations of this SNP to skin²³ and eye color (Frudakis, T. et al. Genetics 165, 2071-83 (2003)), we did not detect an association to any of the pigmentation traits studied, other than freckles. This sets rs1042602 apart from the variants in the MC1R gene and 6p25.3, where the association to freckles is accompanied by an association to sun sensitivity and to hair color (FIG. 1). The ancestral C allele of rs1042602 is fixed in the East Asian and YRI HapMap samples, whereas the A allele is found at a frequency of approximately 35% in European populations. There is strong evidence that rs1042602 A (associated to the absence of freckles) has been subject to positive selection in European populations. Thus, only 1.7% of comparable HapMap SNPs show greater differences in frequency between the CEU and YRI samples and only 0.37% show greater differences between the CEU and East Asian samples. Moreover, only 0.55% of alleles of the same frequency in the HapMap CEU samples have greater or equal irEHH values.

The second SNP chosen for replication in the TYR gene, rs1393350 is strongly correlated with the SNP rs1126809, which codes for a non-synonimous R402Q mutation (D′=1 and r²=0.86). A suggestive association of the A allele of rs1393350 to blue vs. green eye color (OR=1.52, P=2.0×10⁻⁶) in the Icelandic discovery sample was confirmed in the replication samples (combined P=3.3×10⁻¹², Table 2). For this SNP, the greatest difference in allele frequency is between blue and green-eyed individuals, with brown-eyed individuals having an intermediate frequency (FIG. 1). In addition to the primary association to eye color, secondary suggestive associations to blond vs. brown hair and skin sensitivity to sun were also detected (Tables 3 and 4). However, despite the pleiotropic impact of rs1393350 on pigmentation traits, we found no evidence for the action of positive selection based on population divergence or extended haplotype homozygosity.

SLC24A4 Region

Three SNPs (rs4904864, rs4904868 and rs2402130) in a 37 kb region on chromosome 14 show genome-wide significant association to blond vs. brown hair and blue vs. green eyes in the Icelandic discovery sample (Table 5). This region is located within a single LD block that contains the first exons of the gene SLC24A4. No common SNPs at SLC24A5 are available in our dataset; all SNPs in the region have frequency less than 1%.

Analysis of two-SNP haplotypes from the Illumina 317K chip within the LD block revealed that the haplotypic combination of rs4904868 C and rs2402130 A has a stronger and more significant association to the pigmentation traits than any of the three individual SNPs (OR=2.56, P=8.5×10⁻²⁴ blond vs. brown hair and OR=2.06, P=2.0×10⁻¹⁸ blue vs. green eyes) in the Icelandic discovery sample. This haplotype almost accounts completely for the association signal provided by the three SNPs individually, with adjusted association P values greater than 0.25, except for the association of rs4904868 to blond vs. brown hair (P=0.032). An analysis of the HapMap data revealed that the haplotype tags (r²=1) a group of equivalent SNP alleles (rs12896399 rs4904866 T, rs1885194 C and rs17184180 A) that are at 60% frequency in the CEU sample, but less than 1% in the YRI sample. The T allele of rs12896399 shows a similarly strong association to blond vs. brown hair and blue vs. green eyes in the Icelandic and Dutch replication samples as in the Icelandic discovery sample (Tables 2 and 3).

The high frequency of rs12896399 T in the CEU HapMap sample relative to the frequency in the YRI HapMap sample (2.1% of autosomal SNPs in HapMap show a greater difference infrequencies) and the low diversity of CEU haplotypes carrying this allele (6.4% of alleles found at 60% frequency in the CEU sample had greater irEHH) suggest that it may have been under positive selection in European populations.

Note that in the Icelandic and Dutch samples, the greatest difference in allele frequency for rs12896399 is between blue and green eyed individuals similarly to the second TYR variant (FIG. 1).

OCA2-HERC2 Region

A total of 16 SNPs, spanning 1 Mb on chromosome 15, showed genome-wide significant association to blue vs. brown eyes, blue vs. green eyes, blond vs. brown hair, or some combination of these traits in the Icelandic sample (Table 5). This region overlaps with the well-known OCA2 gene, from which rare mutations have long been known to be a major cause of albinism (Sturm, R. A. & Frudakis, T. N. Trends Genet 20, 327-32 (2004); Frudakis, T. et al. Genetics 165, 2071-83 (2003)). A recent study reported three common variants in intron 1 of OCA2 (rs7495174, rs6497268 and rs11855019) that are strongly associated to eye, hair and skin pigmentation in populations of European ancestry (Duffy, D. L. et al. Am J Hum Genet 80, 241-52 (2007)). While all three SNPs were among the 16 detected in our genome scan, the strongest signal of association was provided by rs1667394 (OR=35.42, P=1.4×10⁻¹²⁴ blue vs. brown eyes, OR=7.02, P=5.1×10⁻²⁵ blue vs. green eyes, OR=5.62, P=4.4×10⁻¹⁶ blond vs. brown hair), located 200 kb downstream of OCA2, within intron 4 of the HERC2 gene. For each of the three pigmentation traits, the association to rs1667394 was stronger in the Icelandic discovery sample than the association of the three previously reported SNPs individually. Furthermore, rs1667394 remained significant after adjusting for all haplotypes over the other three SNP, showing that the signal conferred by this marker is singificant on its own. As the link between OCA2 and pigmentation is well-established, it is plausible that the association signal provided by rs1667394 is due to an effect on expression of OCA2 or possibly that presently unidentified functional variants exist within OCA2 that correlate with rs1667394. Due to the fact that the signal is far outside the OCA2 gene, it is also quite possible that the sequence variation in the introns of HERC2 affect the expression or function of HERC2 in a manner that is independent of the effect of sequence variants over the OCA2 gene affecting its function.

The pattern of association exhibited by rs1667394 A to hair and eye color is one of a gradient of reduced pigmentation, with the lowest allele frequency in brown-haired and brown-eyed individuals and the highest frequency in blond-haired and blue-eyed individuals. We note that the same kind of gradient is observed for the association of rs1393350 A in TYR and rs12896399 T in SLC24A4 to hair color, but not to eye color (FIG. 1). Also it is interesting that the nominal association to skin sensitivity to sun observed in both the TYR and SLC24A4 variants is not present for the OCA2 variants, in spite of OCA2 showing stronger association to both eye and hair color (FIG. 1 and Table 4).

The A allele of rs1667394 is found at a frequency of 80-90% in northern European populations. Several studies have reported an extremely strong signal of positive selection acting on the pigmentation reducing variants in OCA2 in populations of European ancestry (Lao, O., et al. Ann Hum Genet (2007); McEvoy, B., et al. Hum Mol Genet 15 Spec No 2, R176-81 (2006); Myles, S., et al. Hum Genet 120, 613-21 (2007)). Similarly, we find that only 0.54% of HapMap SNPs show greater divergence than rs1667394 between the CEU and YRI samples and 0.66% of HapMap SNPs show greater divergence between the CEU and East Asian samples. Furthermore, only 0.32% of HapMap SNPs in the CEU sample have an irEHH value that is greater than or equal to that observed for rs1667394 A.

KITLG Region

A single SNP on 12q21.33, rs12821256, was genome-wide significant in the initial scan for association to blond vs. brown hair (OR=2.32, P=1.9×10⁻¹⁴). This association was confirmed in both replication samples (Table 3). The gene nearest to rs12821256 is KITLG (encodes the ligand for KIT receptor tyrosine kinase), a gene that plays a role in controlling the migration, survival and proliferation of melanocytes (Wehrle-Haller, B. Pigment Cell Res 16, 287-96 (2003)). Rare mutations in the mouse homologue of KITLG are known to affect coat color (Seitz, J. J., et al., Mamm Genome 10, 710-2 (1999)), but no association to pigmentation has hitherto been reported for the human gene (Wehrle-Haller, B. Pigment Cell Res 16, 287-96 (2003)). This SNP lies 350 kb upstream of KITLG and may affect the expression of the gene, or may be in LD with a SNP that affects its expression. This idea is supported by the fact that the mouse homologue of KITLG is regulated by a region that is 100-300 kb upstream of the gene (Wehrle-Haller, B. Pigment Cell Res 16, 287-96 (2003)).

Three recent studies uncovered a strong signal of positive selection in both European and East Asian populations at KITLG (Lao, O., et al. Ann Hum Genet (2007); McEvoy, B., et al. Hum Mol Genet 15 Spec No 2, R176-81 (2006); Williamson, S. H. et al. PLoS Genet 3, e90 (2007); Izagirre, N., et al. Mol Biol Evol 23, 1697-706 (2006)). This signal stems from an extended haplotype spanning a 400 kb region centered on the gene and is found at frequencies of 80%, 63% and 3% in the CEU, East Asian and YRI HapMap samples, respectively. We did not find alleles tagging this haplotype to be consistently associated to any of the six pigmentation traits. Interestingly, the blond hair associated allele rs12821256 C is found almost exclusively on the background of this extended haplotype in populations of European ancestry (at approximately 15% frequency), but is not present in the YRI or East Asian HapMap samples. Only 1.65% of alleles at the same frequency in the CEU HapMap sample have greater or equal irEHH values.

However, the irEHH value of rs12821256 C is substantially reduced when examined only on the background of the extended haplotype. Thus, rs12821256 C was not itself under positive selection, but rather is a hitch-hiker, driven up in frequency by some selective advantage conferred by the extended haplotype.

Discussion

Although numerous genes have been identified as candidates for pigmentation genes through animal models or linkage to diseases with Mendelian patterns of inheritance, most of the genetic variants contributing to the variability of normal human pigmentation remain unknown. Based on genome-wide association scans, we have identified several new variants that account for differences in the pigmentation of eyes, hair and skin among individuals of European ancestry. Except for 6p25.3, these variants are located within or nearby genes that have either been proposed by others as pigmentation candidate genes, KITLG and TYR, or have homology to known candidates, SLC24A4.

Each of these variants can be viewed as having a high minor allele frequency and a moderate effect on pigmentation in Europeans with allelic ORs in the range of 1.2-2.5. This contrasts with the rather large effect but lower minor allele frequency of variants from the remaining two genes detected in our genome scan, MC1R and OCA2, that were described in previous reports (Valverde, P., et al. Nat Genet 11, 328-30 (1995); Eiberg, H. & Mohr, J. Eur J Hum Genet 4, 237-41 (1996)). It is also fascinating to note the apparent differences in the observed association of the different variants to the pigmentation characteristics, with some variants associating to many characteristics and others only one, for instance the striking difference in the pattern of association to eye color for the TYR and SLC24A4 variants when compared to those of OCA2, and the difference in the direction of association to blond hair color between the MC1R variants and the 6p25.3 variants both of which associate to sensitive skin and freckles (FIG. 1). These patterns of association play a substantial role in creating the differences of hair, eye and skin pigmentation compositions observed between individuals in European populations. Our data on pigmentation characteristics are based on self assessment and it is likely that more objective measurement techniques would strengthen the observed associations, and potentially lead to further discoveries.

Given this new set of genetic determinants of pigmentation we have attempted to predict eye and hair pigmentation based on genotypes (FIG. 2, Table 8). For eye colour, the prediction of blue vs. brown eye colour is dominated by variants from the OCA2 region, while other variants described herein add resolution to discriminate between blue and green eye color. For hair color, the contribution of the variants described herein is quite substantial. While red hair color prediction is solely based on MC1R variants, variants in the other regions add predictive power in distinguishing the shades of non-red hair. Red and either blond or brown hair color can be excluded with a high degree of certainty for a substantial proportion of individuals.

It has long been thought that prior to the migrations that first brought our species out of Africa some 60,000 years ago, ancestral human populations were characterized by darkly pigmented skin, eyes and hair (McEvoy, B., et al. Hum Mol Genet 15 Spec No 2, R176-81 (2006)). This notion is consistent with the relatively strong positive correlation in humans between the level of pigmentation of skin and proximity to the equator (Relethford, J. H. Am J Phys Anthropol 104, 449-57 (1997); Sturm, R. A. Trends Genet 22, 464-8 (2006)) and findings that some genes involved in the synthesis of eumelanin are under strong purifying selection in populations exposed to high levels of UVR (Harding, R. M. et al. Am J Hum Genet 66, 1351-61 (2000)). More recently, several studies have provided evidence in support of the idea that positive selection drove to near fixation lighter skin pigmentation in populations at northerly latitudes, such as those of European and East Asian ancestry (Lamason, R. L. et al. Science 310, 1782-6 (2005); Lao, O., et al. Ann Hum Genet (2007); McEvoy, B., et al. Hum Mol Genet 15 Spec No 2, R176-81 (2006); Myles, S., et al. Hum Genet 120, 613-21 (2007)). Our results support this conclusion, in that most of the pigmentation variants discovered in this study show signals of positive selection in European populations. In each case it is the variant that is likely to contribute to lighter pigmentation of the skin that has been swept to high frequency, consistent with positive selection on sequence variants undermining the formation of pigments. The most obvious functional advantage of lighter skin pigmentation in northerly latitudes is that it allows for the synthesis of Vitamin D3 in spite of low levels of UVR exposure (Jablonski, N. G. & Chaplin, G. J Hum Evol 39, 57-106 (2000)). However, other functional advantages or constraints cannot be ruled out. Allele frequency of variants described herein among different populations is shown in Table 9.

The growing number of known sequence variants underlying differences in normal human pigmentation within and between populations may provide new inroads into the molecular physiology of these traits, which in turn could enhance our understanding of how they evolved. At the very least, the newly discovered genetic determinants of human pigmentation provide promising candidates for forensic geneticists and studies of diseases of the skin and eyes that are known to be correlated with such traits.

TABLE 1
Frequencies in percentages of eye, hair and skin pigmentation types among Icelandic
and Dutch individuals.

| | Iceland Discovery | | Iceland Replication | | Holland | |
|---|---|---|---|---|---|---|
| | (N = 2,986) | | (N = 2,718) | | (N = 1,214) | |
| | Male | Female | Male | Female | Male | Female |
| Pigmentation type | (N = 911) | (N = 2,075) | (N = 1,153) | (N = 1,565) | (N = 696) | (N = 518) |
| Eye | | | | | | |
| Blue or grey | 80.0 | 70.3 | 79.6 | 68.2 | 69.5 | 52.3 |
| Green | 8.0 | 17.9 | 9.7 | 21.0 | 5.6 | 17.2 |
| Brown or Black | 9.9 | 10.3 | 8.1 | 8.6 | 19.1 | 24.3 |
| Other or unknown | 2.1 | 1.5 | 2.6 | 2.2 | 5.7 | 7.4 |
| Hair | | | | | | |
| Red or reddish | 6.1 | 8.1 | 5.9 | 7.6 | 1.9 | 3.3 |
| Blond | 15.3 | 15.2 | 14.7 | 17.4 | 22.1 | 19.7 |
| Dark blond or light | 50.8 | 48.1 | 53.2 | 45.8 | 50.9 | 50.2 |
| brown | | | | | | |
| Dark brown or black | 26.1 | 26.3 | 23.9 | 28.1 | 25.0 | 26.8 |
| Unknown | 1.6 | 2.3 | 2.3 | 1.1 | 0.1 | 0.0 |
| Skin sensitivity to sun* | | | | | | |
| Positive | 29.3 | 35.5 | 29.0 | 34.2 | 36.5 | 46.5 |
| Negative | 66.0 | 58.6 | 66.3 | 59.6 | 63.3 | 53.5 |
| Unknown | 4.7 | 5.9 | 4.7 | 6.1 | 0.2 | 0.0 |
| Freckles | | | | | | |
| Present | 38.4 | 50.8 | 42.8 | 60.3 | 29.3 | 45.2 |
| Absent | 57.3 | 45.4 | 55.5 | 38.3 | 70.1 | 54.1 |
| Unknown | 4.3 | 3.7 | 1.6 | 1.3 | 0.6 | 0.8 |

*Based on the Fitzpatrick score in the Icelandic samples. Estimated from related questions in the Dutch sample (see Methods).

TABLE 2
Association of genetic variants to eye color in 2,986 Icelandic discovery individuals,
2,718 Icelandic replication individuals and 1,214 Dutch replication individuals.

| | | | Iceland | | | |
|---|---|---|---|---|---|---|
| | | | Discovery | Replication | Holland | |
| | Locus | Variant | OR (95% c.i.) | OR (95% c.i.) | OR (95% c.i.) | P |
| Blue vs. brown eyes | SLC24A4 | rs12896399 T | 1.15 (0.95, 1.38) | 1.29 (1.05, 1.59) | 1.12 (0.91, 1.36) | 0.032 |
| | KITLG | rs12821256 C | 1.13 (0.89, 1.42) | 1.20 (0.92, 1.56) | 0.96 (0.71, 1.30) | 0.31 |
| | 6P25.3 | rs1540771 A | 1.11 (0.93, 1.34) | 1.18 (0.95, 1.46) | 1.07 (0.87, 1.30) | 0.10 |
| | TYR | rs1393350 A | 1.20 (0.98, 1.47) | 1.27 (1.01, 1.60) | 1.18 (0.94, 1.48) | 0.0044 |
| | | rs1042602 C | 1.01 (0.83, 1.24) | 0.99 (0.78, 1.25) | 0.97 (0.79, 1.19) | 1.00 |
| | OCA2 | rs1667394 A | 29.43 (21.47, 40.35) | 18.46 (12.93, 26.35) | 15.34 (10.75, 21.88) | 1.3 × 10⁻²⁴¹ |
| | | rs7495174 A | 6.90 (3.85, 12.39) | 5.56 (3.02, 10.23) | 4.87 (2.43, 9.74) | 3.0 × 10⁻²⁴ |
| | MC1R | rs1805008 T | 1.15 (0.87, 1.52) | 1.02 (0.77, 1.35) | 1.29 (0.88, 1.89) | 0.20 |
| | | rs1805007 T | 1.37 (0.98, 1.93) | 0.95 (0.70, 1.28) | 0.90 (0.60, 1.36) | 0.044 |
| Blue vs. green eyes | SLC24A4 | rs12896399 T | 2.06 (1.76, 2.42) | 1.49 (1.27, 1.73) | 2.08 (1.58, 2.74) | 4.1 × 10⁻³⁸ |
| | KITLG | rs12821256 C | 0.92 (0.76, 1.11) | 1.09 (0.90, 1.33) | 1.18 (0.78, 1.80) | 0.34 |
| | 6P25.3 | rs1540771 A | 0.99 (0.85, 1.16) | 1.14 (0.98, 1.33) | 0.88 (0.68, 1.15) | 0.59 |
| | TYR | rs1393350 A | 1.52 (1.28, 1.81) | 1.43 (1.21, 1.71) | 1.38 (1.01, 1.89) | 3.3 × 10⁻¹² |
| | | rs1042602 C | 1.08 (0.91, 1.27) | 0.88 (0.74, 1.05) | 1.16 (0.88, 1.52) | 0.11 |
| | OCA2 | rs1667394 A | 6.74 (4.61, 9.83) | 5.83 (4.07, 8.36) | 5.96 (3.48, 10.21) | 1.5 × 10⁻⁵³ |
| | | rs7495174 A | 1.41 (0.75, 2.62) | 2.02 (1.12, 3.65) | 1.45 (0.52, 4.01) | 0.11 |
| | MC1R | rs1805008 T | 1.04 (0.83, 1.31) | 0.85 (0.69, 1.04) | 0.87 (0.55, 1.37) | 0.92 |
| | | rs1805007 T | 0.94 (0.73, 1.22) | 0.74 (0.59, 0.92) | 1.12 (0.63, 1.98) | 0.73 |

TABLE 3
Association of genetic variants to hair color in 2,986 Icelandic discovery individuals,
718 Icelandic replication individuals and 1,214 Dutch replication individuals.

| | | | Iceland | | | |
|---|---|---|---|---|---|---|
| | | | Discovery | Replication | Holland | |
| | Locus | Variant | OR (95% c.i.) | OR (95% c.i.) | OR (95% c.i.) | P |
| Red vs. not red hair | SLC24A4 | rs12896399 T | 1.06 (0.85, 1.31) | 1.07 (0.85, 1.34) | 0.88 (0.52, 1.49) | 0.56 |
| | KITLG | rs12821256 C | 1.01 (0.78, 1.31) | 0.88 (0.67, 1.17) | 0.65 (0.27, 1.55) | 0.84 |
| | 6P25.3 | rs1540771 A | 1.01 (0.82, 1.24) | 1.18 (0.94, 1.48) | 1.05 (0.63, 1.76) | 0.88 |
| | TYR | rs1393350 A | 1.04 (0.83, 1.30) | 1.05 (0.82, 1.34) | 0.79 (0.43, 1.45) | 0.81 |
| | | rs1042602 C | 0.86 (0.69, 1.07) | 0.98 (0.77, 1.27) | 1.21 (0.71, 2.07) | 0.14 |
| | OCA2 | rs1667394 A | 0.91 (0.58, 1.44) | 0.81 (0.49, 1.33) | 1.44 (0.53, 3.96) | 0.83 |
| | | rs7495174 A | 1.49 (0.70, 3.18) | 1.26 (0.58, 2.73) | 1.15 (0.23, 5.73) | 0.16 |
| | MC1R | rs1805008 T | 7.86 (5.96, 10.36) | 4.53 (3.55, 5.77) | 3.71 (1.85, 7.43) | 4.2 × 10⁻⁹⁵ |
| | | rs1805007 T | 12.47 (9.37, 16.60) | 6.12 (4.78, 7.82) | 13.02 (7.02, 24.16) | 2.0 × 10⁻¹⁴² |
| Blond vs. brown hair | SLC24A4 | rs12896399 T | 2.56 (2.12, 3.09) | 2.34 (1.94, 2.82) | 1.86 (1.47, 2.36) | 1.4 × 10⁻⁴⁸ |
| | KITLG | rs12821256 C | 2.32 (1.86, 2.89) | 1.90 (1.52, 2.38) | 2.43 (1.67, 3.54) | 3.8 × 10⁻³⁰ |
| | 6P25.3 | rs1540771 A | 0.69 (0.58, 0.82) | 0.85 (0.71, 1.03) | 0.92 (0.73, 1.17) | 1.1 × 10⁻⁷ |
| | TYR | rs1393350 A | 1.29 (1.06, 1.56) | 1.36 (1.12, 1.66) | 1.22 (0.94, 1.59) | 0.00011 |
| | | rs1042602 C | 0.85 (0.70, 1.03) | 0.81 (0.66, 1.00) | 0.94 (0.74, 1.20) | 0.021 |
| | OCA2 | rs1667394 A | 4.94 (3.16, 7.71) | 5.96 (3.73, 9.52) | 5.51 (3.49, 8.69) | 5.5 × 10⁻³⁵ |
| | | rs7495174 A | 1.92 (0.95, 3.90) | 1.84 (0.86, 3.95) | 0.82 (0.40, 1.68) | 0.070 |
| | MC1R | rs1805008 T | 1.88 (1.40, 2.51) | 1.74 (1.33, 2.28) | 1.93 (1.25, 2.96) | 2.2 × 10⁻¹¹ |
| | | rs1805007 T | 2.34 (1.69, 3.24) | 2.00 (1.52, 2.64) | 1.59 (0.95, 2.66) | 1.9 × 10⁻¹³ |

TABLE 4
Association of genetic variants to skin sensitivity to sun and freckles in 2,986 Icelandic
discovery individuals, 2,718 Icelandic replication individuals and 1,214 Dutch replication
individuals.

| | | | Iceland | | | |
|---|---|---|---|---|---|---|
| | | | Discovery | Replication | Holland | |
| | Locus | Variant | OR (95% c.i.) | OR (95% c.i.) | OR (95% c.i.) | P |
| Skin sensitivity to sun | SLC24A4 | rs12896399 T | 1.21 (1.07, 1.36) | 1.04 (0.92, 1.18) | 0.98 (0.84, 1.16) | 0.00035 |
| | KITLG | rs12821256 C | 1.07 (0.93, 1.24) | 1.22 (1.05, 1.42) | 0.84 (0.66, 1.08) | 0.71 |
| | 6P25.3 | rs1540771 A | 1.21 (1.08, 1.36) | 1.12 (0.99, 1.26) | 1.12 (0.95, 1.32) | 4.0 × 10⁻⁶ |
| | TYR | rs1393350 A | 1.26 (1.11, 1.43) | 1.49 (1.31, 1.70) | 1.11 (0.92, 1.34) | 1.6 × 10⁻⁶ |
| | | rs1042602 C | 0.96 (0.85, 1.09) | 1.05 (0.91, 1.20) | 0.87 (0.73, 1.02) | 0.12 |
| | OCA2 | rs1667394 A | 1.24 (0.95, 1.62) | 1.24 (0.93, 1.65) | 1.34 (1.00, 1.81) | 0.0034 |
| | | rs7495174 A | 1.30 (0.87, 1.96) | 0.99 (0.64, 1.53) | 1.65 (1.03, 2.63) | 0.17 |
| | MC1R | rs1805008 T | 2.30 (1.94, 2.73) | 2.07 (1.77, 2.43) | 1.65 (1.23, 2.20) | 1.8 × 10⁻⁴³ |
| | | rs1805007 T | 2.94 (2.42, 3.58) | 2.51 (2.11, 2.98) | 2.01 (1.44, 2.81) | 1.8 × 10⁻⁵⁵ |
| Freckles | SLC24A4 | rs12896399 T | 0.99 (0.88, 1.11) | 1.04 (0.92, 1.16) | 1.03 (0.87, 1.22) | 1.00 |
| | KITLG | rs12821256 C | 0.89 (0.78, 1.02) | 1.01 (0.88, 1.17) | 0.96 (0.75, 1.24) | 0.074 |
| | 6P25.3 | rs1540771 A | 1.40 (1.26, 1.57) | 1.25 (1.11, 1.40) | 1.26 (1.06, 1.49) | 3.7 × 10⁻¹⁸ |
| | TYR | rs1393350 A | 1.13 (1.00, 1.28) | 1.13 (1.00, 1.28) | 1.10 (0.91, 1.32) | 0.0029 |
| | | rs1042602 C | 1.32 (1.17, 1.49) | 1.39 (1.22, 1.58) | 1.23 (1.04, 1.46) | 1.5 × 10⁻¹¹ |
| | OCA2 | rs1667394 A | 1.16 (0.90, 1.48) | 1.09 (0.83, 1.41) | 1.39 (1.02, 1.88) | 0.026 |
| | | rs7495174 A | 0.84 (0.58, 1.21) | 0.82 (0.55, 1.23) | 1.04 (0.65, 1.66) | 0.29 |
| | MC1R | rs1805008 T | 2.63 (2.21, 3.11) | 2.49 (2.11, 2.93) | 2.06 (1.54, 2.76) | 2.8 × 10⁻⁶⁰ |
| | | rs1805007 T | 4.37 (3.56, 5.37) | 2.54 (2.13, 3.04) | 3.96 (2.81, 5.58) | 1.2 × 10⁻⁹⁶ |

TABLE 5
Genome-wide significant SNPs. P values are corrected using genomic controls.

| | | Build 35 | | | |
|---|---|---|---|---|---|
| SNP | Chr | position | P value | OR | Test |
| rs12821256 C | 12 | 87,830,803 | 5.5 × 10⁻¹⁴ | 2.32 | blond vs. brown hair |
| rs4904864 A | 14 | 91,834,272 | 5.9 × 10⁻¹¹ | 0.51 | blond vs. brown hair |
| | | | 1.9 × 10⁻⁸ | 0.63 | blue vs. green eyes |
| rs4904868 T | 14 | 91,850,754 | 2.2 × 10⁻¹³ | 0.50 | blond vs. brown hair |
| | | | 7.5 × 10⁻¹⁴ | 0.56 | blue vs. green eyes |
| rs2402130 G | 14 | 91,870,956 | 3.7 × 10⁻⁹ | 0.47 | blond vs. brown hair |
| rs1584407 A | 15 | 25,830,854 | 1.1 × 10⁻⁷ | 0.55 | blue vs. brown eyes |
| rs2703952 C | 15 | 25,855,576 | 3.7 × 10⁻¹² | 0.39 | blue vs. brown eyes |
| rs728405 G | 15 | 25,873,448 | 1.1 × 10⁻⁹ | 0.5 | blue vs. brown eyes |
| rs4778220 G | 15 | 25,894,733 | 1.2 × 10⁻⁷ | 0.51 | blue vs. brown eyes |
| rs11855019 G | 15 | 26,009,415 | 9.3 × 10⁻³⁶ | 0.17 | blue vs. brown eyes |
| | | | 5.3 × 10⁻⁹ | 0.32 | blond vs. brown hair |
| rs6497268 A | 15 | 26,012,308 | 4.1 × 10⁻⁵⁶ | 0.13 | blue vs. brown eyes |
| | | | 7.7 × 10⁻¹⁰ | 0.37 | blond vs. brown hair |
| | | | 1.5 × 10⁻¹³ | 0.37 | blue vs. green eyes |
| rs7495174 G | 15 | 26,017,833 | 3.2 × 10⁻³⁶ | 0.07 | blue vs. brown eyes |
| rs7183877 A | 15 | 26,039,328 | 1.2 × 10⁻¹⁰ | 0.16 | blond vs. brown hair |
| | | | 8.0 × 10⁻²² | 0.10 | blue vs. green eyes |
| | | | 2.2 × 10⁻⁷² | 0.03 | blue vs. brown eyes |
| rs8028689 C | 15 | 26,162,483 | 7.3 × 10⁻³⁸ | 0.02 | blue vs. brown eyes |
| rs2240204 T | 15 | 26,167,627 | 7.3 × 10⁻³⁸ | 0.02 | blue vs. brown eyes |
| rs8039195 C | 15 | 26,189,679 | 1.5 × 10⁻¹² | 0.21 | blond vs. brown hair |
| | | | 9.1 × 10⁻²² | 0.15 | blue vs. green eyes |
| | | | 8.8 × 10⁻⁹⁹ | 0.03 | blue vs. brown eyes |
| rs16950979 G | 15 | 26,194,101 | 7.3 × 10⁻³⁸ | 0.02 | blue vs. brown eyes |
| rs16950987 A | 15 | 26,199,823 | 7.3 × 10⁻³⁸ | 0.02 | blue vs. brown eyes |
| rs1667394 G | 15 | 26,203,777 | 4.4 × 10⁻¹⁶ | 0.18 | blond vs. brown hair |
| | | | 5.1 × 10⁻²⁵ | 0.14 | blue vs. green eyes |
| | | | 1.4 × 10⁻¹²⁴ | 0.03 | blue vs. brown eyes |
| rs1635168 T | 15 | 26,208,861 | 5.9 × 10⁻²⁸ | 0.06 | blue vs. brown eyes |
| rs17137796 C | 15 | 26,798,209 | 2.4 × 10⁻¹⁰ | 0.55 | blue vs. brown eyes |
| rs11076747 G | 16 | 87,584,526 | 2.7 × 10⁻⁸ | 0.55 | red vs. not red hair |
| rs9921361 G | 16 | 87,821,940 | 4.4 × 10⁻⁹ | 0.17 | red vs. not red hair |
| rs1466540 C | 16 | 87,871,978 | 1.2 × 10⁻⁷ | 0.52 | red vs. not red hair |
| rs2353028 G | 16 | 87,880,179 | 4.4 × 10⁻¹⁰ | 0.39 | red vs. not red hair |
| rs2306633 A | 16 | 87,882,779 | 5.3 × 10⁻¹² | 0.33 | red vs. not red hair |
| rs2353033 T | 16 | 87,913,062 | 1.9 × 10⁻¹⁷ | 0.40 | red vs. not red hair |
| | | | 4.3 × 10⁻¹⁷ | 0.62 | freckles present vs. absent |
| rs889574 C | 16 | 87,914,309 | 4.4 × 10⁻⁸ | 0.72 | freckles present vs. absent |
| rs4347628 C | 16 | 88,098,136 | 2.1 × 10⁻¹² | 2.15 | red vs. not red hair |
| rs382745 C | 16 | 88,131,087 | 4.5 × 10⁻¹³ | 0.66 | freckles present vs. absent |
| rs352935 A | 16 | 88,176,081 | 2.2 × 10⁻¹⁰ | 0.70 | freckles present vs. absent |
| | | | 6.3 × 10⁻¹⁰ | 0.51 | red vs. not red hair |
| rs464349 T | 16 | 88,183,752 | 1.2 × 10⁻¹³ | 0.66 | freckles present vs. absent |
| rs164741 C | 16 | 88,219,799 | 3.6 × 10⁻¹⁵ | 0.61 | burns vs. tans |
| | | | 9.0 × 10⁻³⁹ | 0.25 | red vs. not red hair |
| | | | 1.4 × 10⁻²⁷ | 0.52 | freckles present vs. absent |
| rs460879 T | 16 | 88,240,390 | 6.7 × 10⁻¹⁹ | 0.61 | freckles present vs. absent |
| | | | 2.0 × 10⁻²² | 0.34 | red vs. not red hair |
| rs7188458 G | 16 | 88,253,985 | 7.6 × 10⁻¹² | 0.67 | burns vs. tans |
| | | | 1.6 × 10⁻³⁷ | 0.24 | red vs. not red hair |
| | | | 4.7 × 10⁻²³ | 0.57 | freckles present vs. absent |
| rs459920 C | 16 | 88,258,328 | 9.5 × 10⁻²⁰ | 0.36 | red vs. not red hair |
| | | | 6.2 × 10⁻¹⁶ | 0.63 | freckles present vs. absent |
| rs12443954 G | 16 | 88,268,997 | 8.0 × 10⁻¹⁴ | 0.25 | red vs. not red hair |
| rs258324 A | 16 | 88,281,756 | 1.7 × 10⁻⁹ | 0.11 | red vs. not red hair |
| rs258322 T | 16 | 88,283,404 | 2.0 × 10⁻¹¹ | 1.77 | burns vs. tans |
| | | | 5.6 × 10⁻²⁷ | 3.84 | red vs. not red hair |
| | | | 1.6 × 10⁻¹⁸ | 2.12 | freckles present vs. absent |
| rs3751695 C | 16 | 88,292,050 | 6.0 × 10⁻¹⁴ | 0.4 | red vs. not red hair |
| | | | 1.1 × 10⁻⁷ | 0.66 | burns vs. tans |
| | | | 4.9 × 10⁻⁸ | 0.66 | freckles present vs. absent |
| rs7204478 C | 16 | 88,322,986 | 5.1 × 10⁻⁸ | 0.73 | burns vs. tans |
| | | | 1.1 × 10⁻¹⁴ | 0.65 | freckles present vs. absent |
| | | | 1.4 × 10⁻³⁹ | 0.23 | red vs. not red hair |
| rs1800359 T | 16 | 88,332,762 | 3.5 × 10⁻²² | 0.31 | red vs. not red hair |
| | | | 1.6 × 10⁻¹³ | 0.65 | freckles present vs. absent |
| rs8058895 C | 16 | 88,342,308 | 2.4 × 10⁻¹⁰ | 1.55 | freckles present vs. absent |
| | | | 1.5 × 10⁻¹⁹ | 2.79 | red vs. not red hair |
| rs7195066 C | 16 | 88,363,824 | 4.3 × 10⁻²⁶ | 5.00 | red vs. not red hair |
| rs16966142 T | 16 | 88,378,534 | 1.1 × 10⁻⁹ | 0.09 | red vs. not red hair |
| rs1800286 A | 16 | 88,397,262 | 9.2 × 10⁻¹⁴ | 0.65 | freckles present vs. absent |
| | | | 2.5 × 10⁻²³ | 0.30 | red vs. not red hair |
| rs11861084 A | 16 | 88,403,211 | 2.2 × 10⁻²⁴ | 0.29 | red vs. not red hair |
| | | | 4.1 × 10⁻¹⁵ | 0.64 | freckles present vs. absent |
| rs8060934 C | 16 | 88,447,526 | 1.7 × 10⁻³⁰ | 0.27 | red vs. not red hair |
| | | | 5.2 × 10⁻⁸ | 0.74 | freckles present vs. absent |
| rs4785755 A | 16 | 88,565,329 | 1.4 × 10⁻⁸ | 0.54 | red vs. not red hair |
| rs4408545 T | 16 | 88,571,529 | 2.2 × 10⁻⁴⁴ | 0.17 | red vs. not red hair |
| | | | 2.8 × 10⁻¹³ | 0.65 | burns vs. tans |
| | | | 2.0 × 10⁻²² | 0.57 | freckles present vs. absent |
| rs4238833 T | 16 | 88,578,190 | 3.9 × 10⁻⁵⁵ | 0.18 | red vs. not red hair |
| | | | 1.9 × 10⁻³² | 0.50 | freckles present vs. absent |
| | | | 3.0 × 10⁻¹⁹ | 0.59 | burns vs. tans |
| rs7201721 G | 16 | 88,586,247 | 4.4 × 10⁻¹⁰ | 1.98 | red vs. not red hair |
| rs4785763 C | 16 | 88,594,437 | 2.4 × 10⁻³³ | 0.49 | freckles present vs. absent |
| | | | 1.1 × 10⁻¹⁹ | 0.58 | burns vs. tans |
| | | | 3.2 × 10⁻⁵⁶ | 0.18 | red vs. not red hair |
| rs9936896 T | 16 | 88,596,560 | 1.0 × 10⁻¹¹ | 0.63 | freckles present vs. absent |
| | | | 1.5 × 10⁻¹² | 0.45 | red vs. not red hair |
| | | | 3.4 × 10⁻⁸ | 0.69 | burns vs. tans |
| rs11648785 T | 16 | 88,612,062 | 2.6 × 10⁻¹⁶ | 0.34 | red vs. not red hair |
| | | | 4.1 × 10⁻¹⁰ | 0.67 | burns vs. tans |
| | | | 1.4 × 10⁻¹⁹ | 0.57 | freckles present vs. absent |
| rs2241039 T | 16 | 88,615,938 | 7.7 × 10⁻¹⁰ | 0.69 | burns vs. tans |
| | | | 6.4 × 10⁻²⁴ | 0.28 | red vs. not red hair |
| | | | 7.0 × 10⁻²¹ | 0.58 | freckles present vs. absent |
| rs1048149 C | 16 | 88,638,451 | 5.7 × 10⁻¹⁰ | 0.49 | red vs. not red hair |
| rs2078478 C | 16 | 88,657,637 | 7.4 × 10⁻⁸ | 3.31 | red vs. not red hair |
| rs7196459 G | 16 | 88,668,978 | 2.2 × 10⁻²⁰ | 0.31 | red vs. not red hair |
| | | | 7.3 × 10⁻¹⁵ | 0.53 | freckles present vs. absent |
| | | | 1.1 × 10⁻¹³ | 0.54 | burns vs. tans |
| rs4959270 C | 6 | 402,748 | 2.2 × 10⁻⁸ | 0.73 | freckles present vs. absent |
| rs1540771 G | 6 | 411,033 | 1.9 × 10⁻⁹ | 0.71 | freckles present vs. absent |

TABLE 6
Frequencies in percentages of key SNPs in all phenotypes and all samples. The first line
corresponds to the Icelandic Discovery sample, the second line the Icelandic replication sample,
and the third the Dutch replication sample.

| | | | | | Hair Color | | | | Skin | | | |
|---|---|---|---|---|---|---|---|---|---|---|---|---|
| Variant | | Eye color | | | | | Dark | | sensitive | | Freckles | |
| Locus | All | Blue | Green | Brown | Red | Blond | blond | Brown | Yes | No | Yes | No |
| rs12896399 T | 54.7 | 57.7 | 39.5 | 54.0 | 56.5 | 67.8 | 56.0 | 44.3 | 57.5 | 52.6 | 54.2 | 54.9 |
| | 56.0 | 58.3 | 48.5 | 52.0 | 57.2 | 67.8 | 55.9 | 47.5 | 56.5 | 55.5 | 56.4 | 55.5 |
| | 47.8 | 50.8 | 33.2 | 48.1 | 44.8 | 57.9 | 46.6 | 42.5 | 47.7 | 48.0 | 48.4 | 47.6 |
| rs12821256 C | 19.6 | 19.6 | 20.9 | 17.8 | 19.9 | 27.8 | 20.2 | 14.2 | 20.2 | 19.2 | 18.8 | 20.6 |
| | 20.9 | 21.5 | 20.0 | 18.6 | 19.1 | 26.7 | 21.8 | 16.1 | 23.2 | 19.8 | 20.9 | 20.8 |
| | 12.2 | 12.3 | 10.6 | 12.7 | 8.3 | 15.8 | 13.4 | 7.2 | 11.1 | 12.9 | 11.9 | 12.3 |
| rs1540771 A | 46.8 | 46.9 | 47.1 | 44.2 | 46.9 | 42.5 | 45.2 | 51.7 | 50.0 | 45.2 | 50.8 | 42.4 |
| | 44.2 | 44.9 | 41.6 | 40.8 | 48.1 | 42.7 | 43.2 | 46.6 | 46.1 | 43.3 | 46.7 | 41.3 |
| | 45.4 | 45.3 | 48.4 | 43.8 | 46.7 | 43.4 | 46.2 | 45.4 | 47.0 | 44.3 | 49.1 | 43.4 |
| rs1393350 A | 29.3 | 31.1 | 22.9 | 27.3 | 30.0 | 32.7 | 29.2 | 27.4 | 32.2 | 27.4 | 30.4 | 27.9 |
| | 30.5 | 32.3 | 25.0 | 27.3 | 31.5 | 34.1 | 30.9 | 27.5 | 36.0 | 27.4 | 31.7 | 29.0 |
| | 25.8 | 27.5 | 21.5 | 24.3 | 21.7 | 28.1 | 25.9 | 24.3 | 27.0 | 25.0 | 27.1 | 25.3 |
| rs1042602 C | 70.2 | 70.5 | 68.9 | 70.2 | 67.0 | 67.6 | 70.7 | 71.2 | 69.6 | 70.4 | 73.0 | 67.2 |
| | 72.2 | 71.9 | 74.3 | 72.1 | 71.9 | 69.6 | 72.2 | 73.8 | 73.0 | 72.0 | 75.2 | 68.6 |
| | 62.4 | 62.5 | 59.1 | 63.4 | 66.7 | 63.1 | 60.8 | 64.6 | 60.4 | 63.8 | 65.5 | 60.7 |
| rs1667394 A | 97.6 | 99.0 | 97.5 | 87.5 | 98.2 | 98.8 | 98.1 | 95.5 | 98.1 | 97.2 | 97.4 | 97.6 |
| | 97.4 | 99.1 | 95.7 | 85.2 | 97.6 | 98.8 | 98.2 | 94.7 | 97.6 | 97.3 | 97.2 | 97.6 |
| | 95.0 | 99.2 | 96.5 | 82.4 | 96.7 | 96.9 | 96.0 | 91.4 | 96.9 | 93.7 | 95.8 | 94.5 |
| rs7495174 A | 94.1 | 98.6 | 91.1 | 67.0 | 94.0 | 97.8 | 95.8 | 88.8 | 95.1 | 93.6 | 94.4 | 93.8 |
| | 93.8 | 98.1 | 88.6 | 67.0 | 93.0 | 97.8 | 96.1 | 87.3 | 94.6 | 93.5 | 93.9 | 93.7 |
| | 88.0 | 97.5 | 85.9 | 62.7 | 91.7 | 94.9 | 89.8 | 78.4 | 90.7 | 86.0 | 90.1 | 86.7 |
| rs1805008 T | 13.5 | 13.5 | 13.0 | 12.8 | 34.3 | 13.9 | 12.3 | 8.7 | 18.8 | 10.4 | 18.4 | 9.1 |
| | 12.5 | 12.1 | 13.7 | 11.9 | 35.4 | 13.8 | 11.4 | 8.3 | 18.0 | 9.9 | 17.1 | 7.4 |
| | 8.3 | 8.6 | 9.8 | 6.8 | 24.1 | 10.8 | 7.7 | 5.9 | 10.6 | 6.7 | 12.0 | 6.2 |
| rs1805007 T | 9.6 | 9.7 | 10.4 | 7.8 | 35.7 | 11.8 | 7.0 | 6.1 | 15.1 | 6.4 | 15.1 | 4.5 |
| | 10.3 | 9.8 | 12.8 | 11.2 | 36.4 | 13.1 | 7.5 | 6.9 | 16.8 | 6.8 | 14.0 | 6.0 |
| | 6.1 | 6.1 | 5.5 | 6.7 | 41.7 | 6.7 | 5.0 | 4.3 | 8.5 | 4.4 | 11.4 | 3.1 |

TABLE 7
Results from tests of positive selection based on population differentiation and extended haplotype homozygosity

| | | | | | | | | | | | irEHH |
|---|---|---|---|---|---|---|---|---|---|---|---|
| | | | | | | | | | | | value |
| | | | | | | | FST (perc. rank)^{a} | | | | (perc. |
| | | Gene/ | | Allele frequency | | | CEU-YRI- | | | | rank)^{a} |
| SNP | Allele | Region | Chrom. | CEU | YRI | ASN | ASN | CEU-YRI | CEU-ASN | Y | CEU |
| rs12896399 | T | SLC24A4 | 14 | 0.600 | 0.008 | 0.393 | 0.405 (9.1) | 0.827 (2.1) | 0.086 (45) | 0.462 (14) | 1.02 (6.4) |
| rs12821256 | C | KITLG | 12 | 0.142 | 0.000 | 0.000 | 0.149 (40) | 0.153 (40) | 0.153 (5.1) | 0 (N/A) | 6.96 (1.7) |
| rs1540771 | T | 6p25.3 | 6 | 0.575 | 0.042 | 0.300 | 0.334 (14) | 0.665 (6.3) | 0.154 (30) | 0.234 (35) | 1.29 (4.1) |
| rs1042602 | A | TYR | 11 | 0.417 | 0.000 | 0.000 | 0.484 (0.81) | 0.526 (1.7) | 0.526 (0.37) | 0 (N/A) | 3.29 (0.55) |
| rs1393350 | A | TYR | 11 | 0.192 | 0.000 | 0.000 | 0.205 (16) | 0.212 (12) | 0.212 (3.3) | 0 (N/A) | 0.94 (46) |
| rs7495174 | A | OCA2 | 15 | 0.949 | 0.848 | 0.292 | 0.591 (2.7) | 0.057 (66) | 0.917 (0.79) | 0.629 (12) | 0.5 (1.3) |
| rs1667394 | T | OCA2 | 15 | 0.862 | 0.052 | 0.172 | 0.828 (0.54) | 1.323 (0.54) | 0.953 (0.66) | 0.073 (63) | 11.23 (0.32) |
| rs1805007 | T | MC1R | 16 | 0.142 | 0.000 | 0.000 | 0.149 (41) | 0.153 (40) | 0.153 (5.1) | 0 (N/A) | 3.91 (6.6) |
| rs1805008 | T | MC1R | 16 | 0.108 | 0.000 | 0.000 | 0.112 (50) | 0.115 (31) | 0.115 (16) | 0 (N/A) | 5.37 (6.2) |

^{a}The percentile rank represents the percent of HapMap alleles of the same frequency in the groups examined that have a value of FST or irEHH that is greater than or equal to that found for the specified allele

TABLE 8
The percentage of the variance of various phenotypes explained
by variants from the MC1R and OCA2 regions, by variants in other
genomic regions (after accounting for the MC1R and OCA2 regions),
and by all the variants combined. All traits were treated as two class
categorical variables, except hair shade which was treated as a
quantitative variable (scoring blond hair as 1, dark blond or light
brown hair as 2, and brown or black hair as 3).

| | | | SLC24A4, | | | |
|---|---|---|---|---|---|---|
| | MC1R and | | TYR, KITLG | | | |
| | OCA2 | | and 6p25.3 | | | |
| | regions | | regions | | All loci | |
| Phenotypes | Iceland | Holland | Iceland | Holland | Iceland | Holland |
| Blue vs. brown | 47.2 | 47.7 | 1.0 | 0.9 | 47.7 | 48.2 |
| eyes | | | | | | |
| Blue vs. green | 7.7 | 10.0 | 4.4 | 5.9 | 11.8 | 15.3 |
| eyes | | | | | | |
| Brown vs. | 26.7 | 17.8 | 0 | 2.7 | 26.7 | 20.0 |
| green eyes | | | | | | |
| Red hair | 29.0 | 26.0 | 0 | 0 | 29.0 | 26.0 |
| Hair shade | 7.2 | 7.3 | 5.9 | 3.2 | 12.7 | 10.2 |
| (minus red) | | | | | | |
| Skin sensitivity | 7.9 | 2.6 | 1.6 | 0.8 | 9.4 | 3.4 |
| to sun | | | | | | |
| Freckles | 9.7 | 8.9 | 1.9 | 1.0 | 11.4 | 9.8 |

TABLE 9
Allele frequency of variants among different populations.

| Locus | SLC24A4 | KITLG | SEC5L1 | TYR | TYR | OCA2 | MC1R | MC1R |
|---|---|---|---|---|---|---|---|---|
| SNP | rs12896399 | rs12821256 | rs1540771 | rs1393350 | rs1042602 | rs1667394 | rs1805008 | rs1805007 |
| Allele | T | C | A | A | C | A | T | T |
| Population N | | | | | | | | |
| Iceland | 54 | 19 | 46 | 29 | 70 | 94 | 14 | 10 |
| 13,264 | | | | | | | | |
| Holland | 48 | 12 | 45 | 25 | 63 | 88 | 8 | 6 |
| 1,214 | | | | | | | | |
| US^{a} | — | 10 | 48 | 26 | 64 | 85 | — | — |
| 2,276 | | | | | | | | |
| CEU^{b} | 60 | 14 | 58 | 19 | 58 | 87 | 8 | 14 |
| 60 | | | | | | | | |
| CHB^{b} | 21 | 0 | 25 | 0 | 100 | 20 | 0 | 0 |
| 45 | | | | | | | | |
| JPT^{b} | 58 | 0 | 36 | 0 | 100 | 14 | 0 | 0 |
| 45 | | | | | | | | |
| YRI^{b} | 1 | 0 | 5 | 0 | 100 | 5 | 0 | 0 |
| 60 | | | | | | | | |
| Pigment | Blond | Blond | Freckle | Blue vs. | Freckle | Blond | Red hair | Red hair |
| effect | Blue vs. | | | green eye | | Blue eye | Fair skin | Fair skin |
| | green eye | | | | | | Freckles | Freckles |

^{a}Cancer Genetic Markers of Susceptibility (CGEMS)
^{b}Hapmap populations

### Example 2

### Identification of Additional Variants Associated with Hair, Eye and Skin Pigmentation

A follow-up analysis of a genome-wide association scan for sequence variants influencing hair color, eye color, freckles and skin sensitivity to sun was performed. Methods used were as described in Example 1 described in detail in the above, with the primary difference that a total of 4611 individuals from the Icelandic population were analyzed.

Results

In Table 10, we shows results of all SNPs that were found to be associated with at least one pigmentation trait to a genome-wide significant level, as defined by the threshold of P<1×10⁻⁷. All the markers indicated in the Table are thus useful for predicting at least one pigmentation trait, and are thus useful in the Methods described herein. Furthermore, we identified all markers that are in linkage disequilibrium with at least one of the markers shown in Table 10. As discussed in detail in the foregoing, markers that are in linkage disequilibrium with markers showing association to a trait are equally useful in methods utilizing those markers. The markers listed in Table 11 below can thus all be utilized to practice the present invention, as they are all highly correlated with the markers shown to be associated with at least one pigmentation trait, as shown in Table 10, and in the Tables 2-5 above.

TABLE 10
Results of a scan for variants associated with pigmentation. Shown are genome-wide significant SNPs. P values are corrected using
genomic controls.

| | | N | f | N | f | | | | | | | SEQ |
|---|---|---|---|---|---|---|---|---|---|---|---|---|
| | | group | group | group | group | Al- | | | | | | ID |
| P-value | OR | 1 | 1 | 2 | 2 | lele | SNP | Chr | Position | Pigmentation trait | Comparison groups | NO |

A. Variants on chromosome 16 (MC1R region)

| | | | | | | | | | | | | |
|---|---|---|---|---|---|---|---|---|---|---|---|---|
| 3.40E−08 | 1.757 | 308 | 0.773 | 1193 | 0.659 | 4 | rs8062328 | chr16 | 87343542 | hair color | red vs brown hair | 118 |
| 5.76E−08 | 0.615 | 335 | 0.567 | 1272 | 0.680 | 3 | rs4782509 | chr16 | 87354279 | hair color | red vs brown hair | 76 |
| 4.27E−09 | 0.533 | 335 | 0.170 | 1269 | 0.278 | 4 | rs4782497 | chr16 | 87546780 | hair color | red vs brown hair | 75 |
| 9.01E−11 | 0.558 | 335 | 0.566 | 1262 | 0.700 | 2 | rs9932354 | chr16 | 87580066 | hair color | red vs brown hair | 131 |
| 3.41E−08 | 0.635 | 335 | 0.566 | 4314 | 0.672 | 2 | rs9932354 | chr16 | 87580066 | hair color | red vs nonred hair | 131 |
| 4.82E−14 | 0.502 | 335 | 0.585 | 1273 | 0.738 | 3 | rs11076747 | chr16 | 87584526 | hair color | red vs brown hair | 8 |
| 4.79E−11 | 0.578 | 335 | 0.585 | 4342 | 0.709 | 3 | rs11076747 | chr16 | 87584526 | hair color | red vs nonred hair | 8 |
| 8.54E−10 | 1.735 | 333 | 0.438 | 1271 | 0.310 | 4 | rs7498845 | chr16 | 87594028 | hair color | red vs brown hair | 106 |
| 1.07E−08 | 1.877 | 335 | 0.870 | 4328 | 0.781 | 4 | rs12599126 | chr16 | 87733984 | hair color | red vs nonred hair | 14 |
| 4.54E−12 | 4.382 | 334 | 0.976 | 1272 | 0.903 | 4 | rs9921361 | chr16 | 87821940 | hair color | red vs brown hair | 130 |
| 6.39E−11 | 3.850 | 334 | 0.976 | 4338 | 0.914 | 4 | rs9921361 | chr16 | 87821940 | hair color | red vs nonred hair | 130 |
| 8.38E−09 | 2.996 | 335 | 0.964 | 1273 | 0.900 | 3 | rs4785648 | chr16 | 87855978 | hair color | red vs brown hair | 78 |
| 8.03E−08 | 2.664 | 335 | 0.964 | 4342 | 0.910 | 3 | rs4785648 | chr16 | 87855978 | hair color | red vs nonred hair | 78 |
| 1.08E−09 | 1.759 | 335 | 0.785 | 4332 | 0.675 | 4 | rs1466540 | chr16 | 87871978 | hair color | red vs nonred hair | 19 |
| 1.41E−13 | 0.409 | 335 | 0.112 | 1273 | 0.236 | 3 | rs2353028 | chr16 | 87880179 | hair color | red vs brown hair | 45 |
| 1.69E−14 | 0.419 | 335 | 0.112 | 4342 | 0.231 | 3 | rs2353028 | chr16 | 87880179 | hair color | red vs nonred hair | 45 |
| 1.13E−17 | 3.059 | 335 | 0.915 | 1271 | 0.779 | 3 | rs2306633 | chr16 | 87882779 | hair color | red vs brown hair | |
| 3.09E−18 | 2.920 | 335 | 0.915 | 4336 | 0.786 | 3 | rs2306633 | chr16 | 87882779 | hair color | red vs nonred hair | |
| 2.64E−09 | 0.423 | 335 | 0.073 | 1272 | 0.157 | 3 | rs3096304 | chr16 | 87901208 | hair color | red vs brown hair | |
| 7.32E−10 | 0.434 | 335 | 0.073 | 4339 | 0.154 | 3 | rs3096304 | chr16 | 87901208 | hair color | red vs nonred hair | |
| 1.43E−25 | 0.644 | 2405 | 0.497 | 2201 | 0.606 | 4 | rs2353033 | chr16 | 87913062 | freckles | freckles vs non-freckles | |
| 1.61E−08 | 0.683 | 689 | 0.515 | 1272 | 0.608 | 4 | rs2353033 | chr16 | 87913062 | hair color | blond vs brown hair | |
| 5.92E−37 | 0.324 | 335 | 0.334 | 1272 | 0.608 | 4 | rs2353033 | chr16 | 87913062 | hair color | red vs brown hair | |
| 2.46E−31 | 0.385 | 335 | 0.334 | 4336 | 0.566 | 4 | rs2353033 | chr16 | 87913062 | hair color | red vs nonred hair | |
| 1.53E−12 | 0.733 | 1675 | 0.505 | 2819 | 0.582 | 4 | rs2353033 | chr16 | 87913062 | skin sun sensitivity | burner vs tanner | |
| 1.42E−27 | 0.548 | 1144 | 0.462 | 1582 | 0.611 | 4 | rs2353033 | chr16 | 87913062 | skin sun sensitivity | freckles/sun sensitive vs | 46 |
| | | | | | | | | | | | non-freckles/not sun | |
| | | | | | | | | | | | sensitive | |
| 8.37E−12 | 1.357 | 2407 | 0.356 | 2204 | 0.289 | 4 | rs889574 | chr16 | 87914309 | freckles | freckles vs non-freckles | 121 |
| 2.59E−10 | 1.447 | 1145 | 0.369 | 1584 | 0.288 | 4 | rs889574 | chr16 | 87914309 | skin sun sensitivity | freckles/sun sensitive vs | 121 |
| | | | | | | | | | | | non-freckles/not sun | |
| | | | | | | | | | | | sensitive | |
| 4.82E−11 | 1.347 | 2407 | 0.338 | 2204 | 0.275 | 4 | rs2965946 | chr16 | 88044113 | freckles | freckles vs non-freckles | 55 |
| 2.09E−08 | 1.393 | 1145 | 0.349 | 1584 | 0.278 | 4 | rs2965946 | chr16 | 88044113 | skin sun sensitivity | freckles/sun sensitive vs | 55 |
| | | | | | | | | | | | non-freckles/not sun | |
| | | | | | | | | | | | sensitive | |
| 1.13E−12 | 0.526 | 335 | 0.315 | 1272 | 0.466 | 4 | rs4347628 | chr16 | 88098136 | hair color | red vs brown hair | 67 |
| 1.09E−15 | 0.512 | 335 | 0.315 | 4336 | 0.473 | 4 | rs4347628 | chr16 | 88098136 | hair color | red vs nonred hair | 67 |
| 4.12E−20 | 1.480 | 2407 | 0.646 | 2200 | 0.552 | 4 | rs382745 | chr16 | 88131087 | freckles | freckles vs non-freckles | 65 |
| 2.17E−16 | 2.141 | 335 | 0.731 | 1272 | 0.560 | 4 | rs382745 | chr16 | 88131087 | hair color | red vs brown hair | 65 |
| 9.45E−14 | 1.904 | 335 | 0.731 | 4338 | 0.588 | 4 | rs382745 | chr16 | 88131087 | hair color | red vs nonred hair | 65 |
| 3.80E−11 | 1.345 | 1676 | 0.642 | 2818 | 0.572 | 4 | rs382745 | chr16 | 88131087 | skin sun sensitivity | burner vs tanner | 65 |
| 3.70E−21 | 1.705 | 1145 | 0.669 | 1580 | 0.542 | 4 | rs382745 | chr16 | 88131087 | skin sun sensitivity | freckles/sun sensitive vs | 65 |
| | | | | | | | | | | | non-freckles/not sun | |
| | | | | | | | | | | | sensitive | |
| 1.51E−08 | 0.522 | 335 | 0.139 | 1273 | 0.236 | 4 | rs3751688 | chr16 | 88161940 | hair color | red vs brown hair | 60 |
| 3.46E−10 | 0.154 | 335 | 0.010 | 1271 | 0.064 | 3 | rs455527 | chr16 | 88171502 | hair color | red vs brown hair | 70 |
| 1.97E−09 | 0.176 | 335 | 0.010 | 4339 | 0.057 | 3 | rs455527 | chr16 | 88171502 | hair color | red vs nonred hair | 70 |
| 2.40E−22 | 1.502 | 2406 | 0.575 | 2203 | 0.473 | 3 | rs352935 | chr16 | 88176081 | freckles | freckles vs non-freckles | 58 |
| 1.04E−18 | 2.210 | 335 | 0.685 | 1272 | 0.496 | 3 | rs352935 | chr16 | 88176081 | hair color | red vs brown hair | 58 |
| 2.37E−18 | 2.070 | 335 | 0.685 | 4340 | 0.512 | 3 | rs352935 | chr16 | 88176081 | hair color | red vs nonred hair | $$ |
| 1.73E−20 | 1.670 | 1145 | 0.603 | 1583 | 0.476 | 3 | rs352935 | chr16 | 88176081 | skin sun sensitivity | freckles/sun sensitive vs | |
| | | | | | | | | | | | non-freckles/not sun | |
| | | | | | | | | | | | sensitive | |
| 5.11E−25 | 0.648 | 2398 | 0.405 | 2196 | 0.513 | 4 | rs464349 | chr16 | 88183752 | freckles | freckles vs non-freckles | |
| 5.89E−18 | 0.459 | 335 | 0.306 | 1268 | 0.490 | 4 | rs464349 | chr16 | 88183752 | hair color | red vs brown hair | |
| 1.11E−16 | 0.499 | 335 | 0.306 | 4325 | 0.469 | 4 | rs464349 | chr16 | 88183752 | hair color | red vs nonred hair | |
| 7.80E−10 | 0.763 | 1669 | 0.417 | 2812 | 0.484 | 4 | rs464349 | chr16 | 88183752 | skin sun sensitivity | burner vs tanner | |
| 1.41E−23 | 0.574 | 1140 | 0.383 | 1578 | 0.520 | 4 | rs464349 | chr16 | 88183752 | skin sun sensitivity | freckles/sun sensitive vs | 73 |
| | | | | | | | | | | | non-freckles/not sun | |
| | | | | | | | | | | | sensitive | |
| 3.38E−08 | 0.774 | 2405 | 0.694 | 2204 | 0.745 | 4 | rs154659 | chr16 | 88194838 | freckles | freckles vs non-freckles | 22 |
| 6.68E−08 | 0.609 | 335 | 0.613 | 1273 | 0.723 | 4 | rs154659 | chr16 | 88194838 | hair color | red vs brown hair | 22 |
| 1.12E−09 | 0.597 | 335 | 0.613 | 4342 | 0.727 | 4 | rs154659 | chr16 | 88194838 | hair color | red vs nonred hair | 22 |
| 8.84E−41 | 1.830 | 2406 | 0.383 | 2200 | 0.253 | 4 | rs164741 | chr16 | 88219799 | freckles | freckles vs non-freckles | 24 |
| 5.74E−70 | 4.885 | 335 | 0.633 | 1273 | 0.261 | 4 | rs164741 | chr16 | 88219799 | hair color | red vs brown hair | 24 |
| 1.85E−68 | 4.174 | 335 | 0.633 | 4337 | 0.292 | 4 | rs164741 | chr16 | 88219799 | hair color | red vs nonred hair | 24 |
| 2.95E−24 | 1.604 | 1675 | 0.380 | 2818 | 0.276 | 4 | rs164741 | chr16 | 88219799 | skin sun sensitivity | burner vs tanner | 24 |
| 1.19E−48 | 2.367 | 1144 | 0.431 | 1580 | 0.242 | 4 | rs164741 | chr16 | 88219799 | skin sun sensitivity | freckles/sun sensitive vs | 24 |
| | | | | | | | | | | | non-freckles/not sun | |
| | | | | | | | | | | | sensitive | |
| 2.85E−29 | 0.625 | 2406 | 0.431 | 2199 | 0.548 | 4 | rs460879 | chr16 | 88240390 | freckles | freckles vs non-freckles | 72 |
| 1.85E−43 | 0.278 | 335 | 0.245 | 1271 | 0.538 | 4 | rs460879 | chr16 | 88240390 | hair color | red vs brown hair | 72 |
| 6.48E−42 | 0.311 | 335 | 0.245 | 4336 | 0.510 | 4 | rs460879 | chr16 | 88240390 | hair color | red vs nonred hair | 72 |
| 2.83E−12 | 0.737 | 1674 | 0.444 | 2818 | 0.520 | 4 | rs460879 | chr16 | 88240390 | skin sun sensitivity | burner vs tanner | 72 |
| 1.79E−28 | 0.542 | 1145 | 0.403 | 1581 | 0.554 | 4 | rs460879 | chr16 | 88240390 | skin sun sensitivity | freckles/sun sensitive vs | 72 |
| | | | | | | | | | | | non-freckles/not sun | |
| | | | | | | | | | | | sensitive | |
| 2.43E−33 | 0.603 | 2407 | 0.484 | 2203 | 0.609 | 3 | rs7188458 | chr16 | 88253985 | freckles | freckles vs non-freckles | 99 |
| 2.44E−08 | 0.686 | 691 | 0.524 | 1272 | 0.616 | 3 | rs7188458 | chr16 | 88253985 | hair color | blond vs brown hair | 99 |
| 1.79E−70 | 0.194 | 335 | 0.237 | 1272 | 0.616 | 3 | rs7188458 | chr16 | 88253985 | hair color | red vs brown hair | 99 |
| 1.01E−64 | 0.233 | 335 | 0.237 | 4341 | 0.572 | 3 | rs7188458 | chr16 | 88253985 | hair color | red vs nonred hair | 99 |
| 8.35E−20 | 0.671 | 1676 | 0.486 | 2821 | 0.585 | 3 | rs7188458 | chr16 | 88253985 | skin sun sensitivity | burner vs tanner | 99 |
| 2.10E−38 | 0.488 | 1145 | 0.443 | 1583 | 0.620 | 3 | rs7188458 | chr16 | 88253985 | skin sun sensitivity | freckles/sun sensitive vs | $$ |
| | | | | | | | | | | | non-freckles/not sun | |
| | | | | | | | | | | | sensitive | |
| 1.49E−24 | 1.534 | 2407 | 0.579 | 2204 | 0.473 | 4 | rs459920 | chr16 | 88258328 | freckles | freckles vs non-freckles | $$ |
| 5.24E−40 | 3.412 | 335 | 0.755 | 1273 | 0.475 | 4 | rs459920 | chr16 | 88258328 | hair color | red vs brown hair | $$ |
| 3.03E−37 | 3.008 | 335 | 0.755 | 4342 | 0.506 | 4 | rs459920 | chr16 | 88258328 | hair color | red vs nonred hair | $$ |
| 6.23E−12 | 1.351 | 1676 | 0.571 | 2822 | 0.496 | 4 | rs459920 | chr16 | 88258328 | skin sun sensitivity | burner vs tanner | $$ |
| 2.32E−25 | 1.779 | 1145 | 0.610 | 1584 | 0.467 | 4 | rs459920 | chr16 | 88258328 | skin sun sensitivity | freckles/sun sensitive vs | $$ |
| | | | | | | | | | | | non-freckles/not sun | |
| | | | | | | | | | | | sensitive | |
| 4.50E−13 | 5.187 | 335 | 0.981 | 1273 | 0.907 | 3 | rs3751700 | chr16 | 88279695 | hair color | red vs brown hair | 61 |
| 1.52E−10 | 4.192 | 335 | 0.981 | 4341 | 0.923 | 3 | rs3751700 | chr16 | 88279695 | hair color | red vs nonred hair | 61 |
| 3.65E−15 | 7.459 | 335 | 0.987 | 1269 | 0.908 | 2 | rs258324 | chr16 | 88281756 | hair color | red vs brown hair | 51 |
| 1.33E−12 | 6.001 | 335 | 0.987 | 4329 | 0.924 | 2 | rs258324 | chr16 | 88281756 | hair color | red vs nonred hair | 51 |
| 8.88E−25 | 1.929 | 2407 | 0.162 | 2203 | 0.091 | 4 | rs258322 | chr16 | 88283404 | freckles | freckles vs non-freckles | 50 |
| 3.80E−46 | 4.740 | 334 | 0.329 | 1273 | 0.094 | 4 | rs258322 | chr16 | 88283404 | hair color | red vs brown hair | 50 |
| 4.82E−46 | 3.919 | 334 | 0.329 | 4342 | 0.111 | 4 | rs258322 | chr16 | 88283404 | hair color | red vs nonred hair | 50 |
| 2.06E−19 | 1.785 | 1676 | 0.167 | 2821 | 0.101 | 4 | rs258322 | chr16 | 88283404 | skin sun sensitivity | burner vs tanner | 50 |
| 3.35E−30 | 2.531 | 1145 | 0.192 | 1583 | 0.086 | 4 | rs258322 | chr16 | 88283404 | skin sun sensitivity | freckles/sun sensitive vs | 50 |
| | | | | | | | | | | | non-freckles/not sun | |
| | | | | | | | | | | | sensitive | |
| 2.74E−10 | 2.568 | 335 | 0.934 | 1272 | 0.847 | 4 | rs1946482 | chr16 | 88289911 | hair color | red vs brown hair | 32 |
| 1.28E−10 | 2.471 | 335 | 0.934 | 4339 | 0.852 | 4 | rs1946482 | chr16 | 88289911 | hair color | red vs nonred hair | 32 |
| 3.22E−09 | 0.608 | 334 | 0.605 | 4333 | 0.716 | 4 | rs6500437 | chr16 | 88317399 | hair color | red vs nonred hair | 95 |
| 6.65E−21 | 1.484 | 2407 | 0.493 | 2204 | 0.396 | 4 | rs7204478 | chr16 | 88322986 | freckles | freckles vs non-freckles | 103 |
| 2.96E−62 | 4.646 | 335 | 0.758 | 1273 | 0.403 | 4 | rs7204478 | chr16 | 88322986 | hair color | red vs brown hair | 103 |
| 2.60E−66 | 4.347 | 335 | 0.758 | 4342 | 0.419 | 4 | rs7204478 | chr16 | 88322986 | hair color | red vs nonred hair | 103 |
| 2.22E−14 | 1.398 | 1676 | 0.497 | 2822 | 0.414 | 4 | rs7204478 | chr16 | 88322986 | skin sun sensitivity | burner vs tanner | 103 |
| 7.92E−27 | 1.809 | 1145 | 0.536 | 1584 | 0.390 | 4 | rs7204478 | chr16 | 88322986 | skin sun sensitivity | freckles/sun sensitive vs | 103 |
| | | | | | | | | | | | non-freckles/not sun | |
| | | | | | | | | | | | sensitive | |
| 1.47E−20 | 0.672 | 2405 | 0.348 | 2200 | 0.443 | 4 | rs1800359 | chr16 | 88332762 | freckles | freckles vs non-freckles | 30 |
| 6.20E−32 | 0.308 | 335 | 0.179 | 1273 | 0.415 | 4 | rs1800359 | chr16 | 88332762 | hair color | red vs brown hair | 30 |
| 1.26E−35 | 0.313 | 335 | 0.179 | 4336 | 0.411 | 4 | rs1800359 | chr16 | 88332762 | hair color | red vs nonred hair | 30 |
| 2.71E−09 | 0.765 | 1673 | 0.354 | 2819 | 0.418 | 4 | rs1800359 | chr16 | 88332762 | skin sun sensitivity | burner vs tanner | 30 |
| 2.66E−21 | 0.583 | 1144 | 0.315 | 1582 | 0.441 | 4 | rs1800359 | chr16 | 88332762 | skin sun sensitivity | freckles/sun sensitive vs | |
| | | | | | | | | | | | non-freckles/not sun | |
| | | | | | | | | | | | sensitive | |
| 2.50E−13 | 0.686 | 2407 | 0.760 | 2204 | 0.822 | 4 | rs8058895 | chr16 | 88342308 | freckles | freckles vs non-freckles | 1 |
| 2.48E−32 | 0.321 | 335 | 0.597 | 1273 | 0.822 | 4 | rs8058895 | chr16 | 88342308 | hair color | red vs brown hair | 1 |
| 5.53E−33 | 0.354 | 335 | 0.597 | 4342 | 0.807 | 4 | rs8058895 | chr16 | 88342308 | hair color | red vs nonred hair | 1 |
| 6.90E−11 | 0.708 | 1676 | 0.754 | 2822 | 0.812 | 4 | rs8058895 | chr16 | 88342308 | skin sun sensitivity | burner vs tanner | 1 |
| 5.93E−19 | 0.552 | 1145 | 0.731 | 1584 | 0.831 | 4 | rs8058895 | chr16 | 88342308 | skin sun sensitivity | freckles/sun sensitive vs | 115 |
| | | | | | | | | | | | non-freckles/not sun | |
| | | | | | | | | | | | sensitive | |
| 8.25E−09 | 1.622 | 335 | 0.396 | 4342 | 0.287 | 2 | rs2011877 | chr16 | 88342319 | hair color | red vs nonred hair | 33 |
| 8.33E−49 | 0.156 | 335 | 0.072 | 1258 | 0.330 | 4 | rs7195066 | chr16 | 88363824 | hair color | red vs brown hair | 100 |
| 4.26E−48 | 0.174 | 335 | 0.072 | 4299 | 0.308 | 4 | rs7195066 | chr16 | 88363824 | hair color | red vs nonred hair | 100 |
| 2.32E−10 | 0.734 | 1667 | 0.252 | 2786 | 0.315 | 4 | rs7195066 | chr16 | 88363824 | skin sun sensitivity | burner vs tanner | 100 |
| 1.96E−13 | 0.635 | 1140 | 0.236 | 1563 | 0.327 | 4 | rs7195066 | chr16 | 88363824 | skin sun sensitivity | freckles/sun sensitive vs | 100 |
| | | | | | | | | | | | non-freckles/not sun | |
| | | | | | | | | | | | sensitive | |
| 4.25E−10 | 1.305 | 2401 | 0.433 | 2200 | 0.369 | 4 | rs2239359 | chr16 | 88376981 | freckles | freckles vs non-freckles | 36 |
| 1.20E−10 | 1.433 | 1143 | 0.454 | 1582 | 0.367 | 4 | rs2239359 | chr16 | 88376981 | skin sun sensitivity | freckles/sun sensitive vs | 36 |
| | | | | | | | | | | | non-freckles/not sun | |
| | | | | | | | | | | | sensitive | |
| 3.84E−15 | 0.089 | 335 | 0.007 | 1273 | 0.078 | 4 | rs16966142 | chr16 | 88378534 | hair color | red vs brown hair | 28 |
| 1.20E−14 | 0.099 | 335 | 0.007 | 4339 | 0.070 | 4 | rs16966142 | chr16 | 88378534 | hair color | red vs nonred hair | 28 |
| 1.93E−21 | 1.500 | 2404 | 0.647 | 2202 | 0.550 | 3 | rs1800286 | chr16 | 88397262 | freckles | freckles vs non-freckles | 29 |
| 2.45E−33 | 3.331 | 335 | 0.821 | 1270 | 0.579 | 3 | rs1800286 | chr16 | 88397262 | hair color | red vs brown hair | 29 |
| 1.57E−37 | 3.295 | 335 | 0.821 | 4337 | 0.582 | 3 | rs1800286 | chr16 | 88397262 | hair color | red vs nonred hair | 29 |
| 2.56E−10 | 1.328 | 1675 | 0.642 | 2818 | 0.575 | 3 | rs1800286 | chr16 | 88397262 | skin sun sensitivity | burner vs tanner | 29 |
| 8.35E−23 | 1.748 | 1145 | 0.682 | 1583 | 0.551 | 3 | rs1800286 | chr16 | 88397262 | skin sun sensitivity | freckles/sun sensitive vs | 29 |
| | | | | | | | | | | | non-freckles/not sun | |
| | | | | | | | | | | | sensitive | |
| 3.89E−23 | 1.524 | 2407 | 0.644 | 2204 | 0.542 | 2 | rs11861084 | chr16 | 88403211 | freckles | freckles vs non-freckles | 12 |
| 5.57E−35 | 3.426 | 335 | 0.821 | 1273 | 0.572 | 2 | rs11861084 | chr16 | 88403211 | hair color | red vs brown hair | 12 |
| 4.34E−39 | 3.374 | 335 | 0.821 | 4342 | 0.576 | 2 | rs11861084 | chr16 | 88403211 | hair color | red vs nonred hair | |
| 3.48E−10 | 1.324 | 1676 | 0.637 | 2822 | 0.570 | 2 | rs11861084 | chr16 | 88403211 | skin sun sensitivity | burner vs tanner | |
| 9.63E−24 | 1.767 | 1145 | 0.678 | 1584 | 0.544 | 2 | rs11861084 | chr16 | 88403211 | skin sun sensitivity | freckles/sun sensitive vs | |
| | | | | | | | | | | | non-freckles/not sun | |
| | | | | | | | | | | | sensitive | |
| 2.17E−08 | 1.264 | 2407 | 0.560 | 2204 | 0.502 | 4 | rs8060934 | chr16 | 88447526 | freckles | freckles vs non-freckles | 1 |
| 5.12E−49 | 4.127 | 335 | 0.803 | 1273 | 0.497 | 4 | rs8060934 | chr16 | 88447526 | hair color | red vs brown hair | 1 |
| 8.45E−53 | 3.943 | 335 | 0.803 | 4342 | 0.508 | 4 | rs8060934 | chr16 | 88447526 | hair color | red vs nonred hair | 1 |
| 8.01E−11 | 1.432 | 1145 | 0.589 | 1584 | 0.500 | 4 | rs8060934 | chr16 | 88447526 | skin sun sensitivity | freckles/sun sensitive vs | 117 |
| | | | | | | | | | | | non-freckles/not sun | |
| | | | | | | | | | | | sensitive | |
| 2.65E−09 | 3.088 | 335 | 0.964 | 1273 | 0.897 | 4 | rs3803688 | chr16 | 88462387 | hair color | red vs brown hair | 64 |
| 1.29E−08 | 2.799 | 335 | 0.964 | 4342 | 0.906 | 4 | rs3803688 | chr16 | 88462387 | hair color | red vs nonred hair | 64 |
| 4.73E−09 | 0.256 | 329 | 0.021 | 1225 | 0.078 | 4 | rs2270460 | chr16 | 88499917 | hair color | red vs brown hair | 40 |
| 1.55E−10 | 0.248 | 329 | 0.021 | 4217 | 0.081 | 4 | rs2270460 | chr16 | 88499917 | hair color | red vs nonred hair | 40 |
| 3.10E−08 | 0.737 | 1144 | 0.509 | 1584 | 0.584 | 4 | rs8045560 | chr16 | 88506995 | skin sun sensitivity | freckles/sun sensitive vs | 114 |
| | | | | | | | | | | | non-freckles/not sun | |
| | | | | | | | | | | | sensitive | |
| 5.97E−09 | 2.972 | 335 | 0.963 | 1273 | 0.897 | 3 | rs3212346 | chr16 | 88509859 | hair color | red vs brown hair | 57 |
| 4.11E−10 | 2.996 | 335 | 0.963 | 4340 | 0.896 | 3 | rs3212346 | chr16 | 88509859 | hair color | red vs nonred hair | 57 |
| 7.83E−11 | 17.208 | 334 | 0.997 | 1272 | 0.951 | 3 | rs885479 | chr16 | 88513655 | hair color | red vs brown hair | 120 |
| 4.66E−10 | 14.925 | 334 | 0.997 | 4336 | 0.957 | 3 | rs885479 | chr16 | 88513655 | hair color | red vs nonred hair | 120 |
| 2.09E−12 | 1.376 | 2406 | 0.339 | 2204 | 0.271 | 3 | rs4785755 | chr16 | 88565329 | freckles | freckles vs non-freckles | 79 |
| 2.91E−13 | 1.933 | 335 | 0.436 | 1273 | 0.286 | 3 | rs4785755 | chr16 | 88565329 | hair color | red vs brown hair | 79 |
| 9.80E−14 | 1.851 | 335 | 0.436 | 4341 | 0.294 | 3 | rs4785755 | chr16 | 88565329 | hair color | red vs nonred hair | 79 |
| 3.61E−09 | 1.319 | 1676 | 0.344 | 2821 | 0.284 | 3 | rs4785755 | chr16 | 88565329 | skin sun sensitivity | burner vs tanner | 79 |
| 9.00E−17 | 1.638 | 1145 | 0.365 | 1584 | 0.260 | 3 | rs4785755 | chr16 | 88565329 | skin sun sensitivity | freckles/sun sensitive vs | 79 |
| | | | | | | | | | | | non-freckles/not sun | |
| | | | | | | | | | | | sensitive | |
| 1.14E−41 | 0.566 | 2406 | 0.379 | 2202 | 0.519 | 4 | rs4408545 | chr16 | 88571529 | freckles | freckles vs non-freckles | 68 |
| 5.89E−72 | 0.153 | 335 | 0.128 | 1273 | 0.490 | 4 | rs4408545 | chr16 | 88571529 | hair color | red vs brown hair | 68 |
| 1.00E−76 | 0.163 | 335 | 0.128 | 4339 | 0.475 | 4 | rs4408545 | chr16 | 88571529 | hair color | red vs nonred hair | 68 |
| 1.53E−24 | 0.636 | 1676 | 0.380 | 2819 | 0.491 | 4 | rs4408545 | chr16 | 88571529 | skin sun sensitivity | burner vs tanner | 68 |
| 1.20E−50 | 0.431 | 1145 | 0.325 | 1582 | 0.527 | 4 | rs4408545 | chr16 | 88571529 | skin sun sensitivity | freckles/sun sensitive vs | |
| | | | | | | | | | | | non-freckles/not sun | |
| | | | | | | | | | | | sensitive | |
| 5.77E−53 | 0.515 | 2381 | 0.533 | 2185 | 0.689 | 4 | rs4238833 | chr16 | 88578190 | freckles | freckles vs non-freckles | |
| 4.92E−91 | 0.154 | 335 | 0.234 | 1260 | 0.666 | 4 | rs4238833 | chr16 | 88578190 | hair color | red vs brown hair | |
| 6.37E−95 | 0.171 | 335 | 0.234 | 4297 | 0.641 | 4 | rs4238833 | chr16 | 88578190 | hair color | red vs nonred hair | |
| 4.43E−33 | 0.585 | 1661 | 0.530 | 2793 | 0.659 | 4 | rs4238833 | chr16 | 88578190 | skin sun sensitivity | burner vs tanner | |
| 6.05E−65 | 0.380 | 1135 | 0.476 | 1568 | 0.705 | 4 | rs4238833 | chr16 | 88578190 | skin sun sensitivity | freckles/sun sensitive vs | |
| | | | | | | | | | | | non-freckles/not sun | |
| | | | | | | | | | | | sensitive | |
| 1.79E−15 | 2.077 | 331 | 0.421 | 1273 | 0.260 | 3 | rs7201721 | chr16 | 88586247 | hair color | red vs brown hair | 102 |
| 8.78E−17 | 2.015 | 331 | 0.421 | 4339 | 0.265 | 3 | rs7201721 | chr16 | 88586247 | hair color | red vs nonred hair | 102 |
| 3.22E−08 | 1.399 | 1138 | 0.322 | 1584 | 0.253 | 3 | rs7201721 | chr16 | 88586247 | skin sun sensitivity | freckles/sun sensitive vs | 102 |
| | | | | | | | | | | | non-freckles/not sun | |
| | | | | | | | | | | | sensitive | |
| 5.74E−52 | 0.515 | 2398 | 0.558 | 2193 | 0.711 | 2 | rs4785763 | chr16 | 88594437 | freckles | freckles vs non-freckles | 80 |
| 4.29E−95 | 0.150 | 335 | 0.252 | 1268 | 0.693 | 2 | rs4785763 | chr16 | 88594437 | hair color | red vs brown hair | 80 |
| 5.05E−98 | 0.170 | 335 | 0.252 | 4322 | 0.665 | 2 | rs4785763 | chr16 | 88594437 | hair color | red vs nonred hair | 80 |
| 5.06E−34 | 0.578 | 1669 | 0.554 | 2808 | 0.682 | 2 | rs4785763 | chr16 | 88594437 | skin sun sensitivity | burner vs tanner | 80 |
| 3.26E−65 | 0.377 | 1140 | 0.498 | 1575 | 0.724 | 2 | rs4785763 | chr16 | 88594437 | skin sun sensitivity | freckles/sun sensitive vs | 80 |
| | | | | | | | | | | | non-freckles/not sun | |
| | | | | | | | | | | | sensitive | |
| 4.37E−21 | 0.624 | 2406 | 0.732 | 2204 | 0.814 | 4 | rs9936896 | chr16 | 88596560 | freckles | freckles vs non-freckles | 133 |
| 1.71E−25 | 0.368 | 334 | 0.609 | 1273 | 0.809 | 4 | rs9936896 | chr16 | 88596560 | hair color | red vs brown hair | 133 |
| 5.31E−23 | 0.426 | 334 | 0.609 | 4342 | 0.785 | 4 | rs9936896 | chr16 | 88596560 | hair color | red vs nonred hair | 133 |
| 9.84E−16 | 0.663 | 1675 | 0.725 | 2822 | 0.799 | 4 | rs9936896 | chr16 | 88596560 | skin sun sensitivity | burner vs tanner | 133 |
| 1.46E−28 | 0.487 | 1144 | 0.697 | 1584 | 0.825 | 4 | rs9936896 | chr16 | 88596560 | skin sun sensitivity | freckles/sun sensitive vs | 133 |
| | | | | | | | | | | | non-freckles/not sun | |
| | | | | | | | | | | | sensitive | |
| 2.79E−11 | 2.757 | 333 | 0.938 | 1260 | 0.847 | 3 | rs8059973 | chr16 | 88607035 | hair color | red vs brown hair | 116 |
| 2.55E−09 | 2.386 | 333 | 0.938 | 4293 | 0.865 | 3 | rs8059973 | chr16 | 88607035 | hair color | red vs nonred hair | 116 |
| 6.49E−08 | 1.618 | 334 | 0.448 | 1269 | 0.334 | 3 | rs9936215 | chr16 | 88609161 | hair color | red vs brown hair | 132 |
| 2.47E−08 | 1.578 | 334 | 0.448 | 4329 | 0.339 | 3 | rs9936215 | chr16 | 88609161 | hair color | red vs nonred hair | 132 |
| 2.37E−27 | 0.613 | 2397 | 0.264 | 2193 | 0.370 | 4 | rs11648785 | chr16 | 88612062 | freckles | freckles vs non-freckles | 11 |
| 1.76E−27 | 0.310 | 334 | 0.142 | 1268 | 0.348 | 4 | rs11648785 | chr16 | 88612062 | hair color | red vs brown hair | 11 |
| 8.53E−27 | 0.338 | 334 | 0.142 | 4322 | 0.329 | 4 | rs11648785 | chr16 | 88612062 | hair color | red vs nonred hair | |
| 8.13E−17 | 0.672 | 1672 | 0.265 | 2805 | 0.349 | 4 | rs11648785 | chr16 | 88612062 | skin sun sensitivity | burner vs tanner | |
| 4.02E−33 | 0.484 | 1142 | 0.231 | 1576 | 0.383 | 4 | rs11648785 | chr16 | 88612062 | skin sun sensitivity | freckles/sun ensitive vs | |
| | | | | | | | | | | | non-freckles/not sun | |
| | | | | | | | | | | | sensitive | |
| 1.72E−31 | 0.604 | 2407 | 0.317 | 2203 | 0.435 | 4 | rs2241039 | chr16 | 88615938 | freckles | freckles vs non-freckles | |
| 3.04E−42 | 0.245 | 335 | 0.151 | 1272 | 0.421 | 4 | rs2241039 | chr16 | 88615938 | hair color | red vs brown hair | |
| 5.62E−40 | 0.276 | 335 | 0.151 | 4341 | 0.392 | 4 | rs2241039 | chr16 | 88615938 | hair color | red vs nonred hair | |
| 1.31E−16 | 0.686 | 1676 | 0.320 | 2821 | 0.407 | 4 | rs2241039 | chr16 | 88615938 | skin sun sensitivity | burner vs tanner | |
| 3.48E−35 | 0.490 | 1145 | 0.285 | 1583 | 0.448 | 4 | rs2241039 | chr16 | 88615938 | skin sun sensitivity | freckles/sun sensitive vs | 39 |
| | | | | | | | | | | | non-freckles/not sun | |
| | | | | | | | | | | | sensitive | |
| 4.36E−09 | 1.776 | 334 | 0.322 | 1273 | 0.211 | 2 | rs4785766 | chr16 | 88629885 | hair color | red vs brown hair | 81 |
| 1.69E−08 | 1.654 | 334 | 0.322 | 4341 | 0.223 | 2 | rs4785766 | chr16 | 88629885 | hair color | red vs nonred hair | 81 |
| 7.62E−08 | 0.797 | 2404 | 0.541 | 2201 | 0.596 | 3 | rs7498985 | chr16 | 88630618 | freckles | freckles vs non-freckles | 107 |
| 2.83E−11 | 7.040 | 335 | 0.990 | 1272 | 0.931 | 3 | rs3785181 | chr16 | 88632834 | hair color | red vs brown hair | 63 |
| 1.56E−11 | 6.643 | 335 | 0.990 | 4341 | 0.934 | 3 | rs3785181 | chr16 | 88632834 | hair color | red vs nonred hair | 63 |
| 5.53E−11 | 0.277 | 335 | 0.031 | 1273 | 0.104 | 4 | rs2241032 | chr16 | 88637020 | hair color | red vs brown hair | 38 |
| 2.35E−10 | 0.305 | 335 | 0.031 | 4341 | 0.096 | 4 | rs2241032 | chr16 | 88637020 | hair color | red vs nonred hair | 38 |
| 1.76E−11 | 1.402 | 2407 | 0.253 | 2204 | 0.194 | 4 | rs1048149 | chr16 | 88638451 | freckles | freckles vs non-freckles | 5 |
| 2.44E−19 | 2.391 | 335 | 0.361 | 1273 | 0.191 | 4 | rs1048149 | chr16 | 88638451 | hair color | red vs brown hair | 5 |
| 1.36E−17 | 2.113 | 335 | 0.361 | 4342 | 0.211 | 4 | rs1048149 | chr16 | 88638451 | hair color | red vs nonred hair | 5 |
| 1.39E−09 | 1.368 | 1676 | 0.258 | 2822 | 0.203 | 4 | rs1048149 | chr16 | 88638451 | skin sun sensitivity | burner vs tanner | 5 |
| 2.50E−17 | 1.735 | 1145 | 0.283 | 1584 | 0.185 | 4 | rs1048149 | chr16 | 88638451 | skin sun sensitivity | freckles/sun sensitive vs | 5 |
| | | | | | | | | | | | non-freckles/not sun | |
| | | | | | | | | | | | sensitive | |
| 3.26E−12 | 1.396 | 2361 | 0.296 | 2172 | 0.231 | 2 | rs4785612 | chr16 | 88640608 | freckles | freckles vs non-freckles | 77 |
| 9.83E−12 | 1.903 | 334 | 0.368 | 1254 | 0.234 | 2 | rs4785612 | chr16 | 88640608 | hair color | red vs brown hair | 77 |
| 3.24E−10 | 1.717 | 334 | 0.368 | 4265 | 0.253 | 2 | rs4785612 | chr16 | 88640608 | hair color | red vs nonred hair | 77 |
| 8.01E−08 | 1.304 | 1645 | 0.298 | 2775 | 0.245 | 2 | rs4785612 | chr16 | 88640608 | skin sun sensitivity | burner vs tanner | 77 |
| 6.42E−15 | 1.629 | 1123 | 0.316 | 1561 | 0.221 | 2 | rs4785612 | chr16 | 88640608 | skin sun sensitivity | freckles/sun sensitive vs | 77 |
| | | | | | | | | | | | non-freckles/not sun | |
| | | | | | | | | | | | sensitive | |
| 5.28E−12 | 0.285 | 334 | 0.037 | 1268 | 0.120 | 4 | rs2078478 | chr16 | 88657637 | hair color | red vs brown hair | 34 |
| 4.35E−13 | 0.289 | 334 | 0.037 | 4320 | 0.119 | 4 | rs2078478 | chr16 | 88657637 | hair color | red vs nonred hair | 34 |
| 1.21E−27 | 1.946 | 2400 | 0.181 | 2191 | 0.102 | 4 | rs7196459 | chr16 | 88668978 | freckles | freckles vs non-freckles | 101 |
| 1.34E−41 | 4.296 | 333 | 0.329 | 1270 | 0.102 | 4 | rs7196459 | chr16 | 88668978 | hair color | red vs brown hair | 101 |
| 1.76E−38 | 3.419 | 333 | 0.329 | 4324 | 0.125 | 4 | rs7196459 | chr16 | 88668978 | hair color | red vs nonred hair | 101 |
| 6.48E−21 | 1.777 | 1666 | 0.187 | 2814 | 0.115 | 4 | rs7196459 | chr16 | 88668978 | skin sun sensitivity | burner vs tanner | 101 |
| 6.33E−38 | 2.742 | 1140 | 0.217 | 1577 | 0.092 | 4 | rs7196459 | chr16 | 88668978 | skin sun sensitivity | freckles/sun sensitive vs | 101 |
| | | | | | | | | | | | non-freckles/not sun | |
| | | | | | | | | | | | sensitive | |

B. Variants on chromosome 15 (OCA2/HERC2 region)

| | | | | | | | | | | | | |
|---|---|---|---|---|---|---|---|---|---|---|---|---|
| 2.19E−09 | 0.656 | 3479 | 0.560 | 490 | 0.660 | 2 | rs1498519 | chr15 | 25685246 | eye color | blue vs brown eyes | 20 |
| 6.89E−09 | 0.667 | 3493 | 0.547 | 491 | 0.645 | 4 | rs6497238 | chr15 | 25727373 | eye color | blue vs brown eyes | 94 |
| 7.44E−12 | 1.764 | 3490 | 0.838 | 491 | 0.745 | 2 | rs1584407 | chr15 | 25830854 | eye color | blue vs brown eyes | 23 |
| 7.95E−10 | 1.442 | 3490 | 0.838 | 1226 | 0.782 | 2 | rs1584407 | chr15 | 25830854 | eye color | blue vs nonblue eyes | 23 |
| 2.95E−17 | 0.419 | 3484 | 0.078 | 485 | 0.168 | 2 | rs2703952 | chr15 | 25855576 | eye color | blue vs brown eyes | 53 |
| 8.70E−15 | 0.553 | 3484 | 0.078 | 1216 | 0.133 | 2 | rs2703952 | chr15 | 25855576 | eye color | blue vs nonblue eyes | 53 |
| 6.95E−13 | 1.736 | 3494 | 0.792 | 491 | 0.686 | 3 | rs2594935 | chr15 | 25858633 | eye color | blue vs brown eyes | 52 |
| 4.64E−11 | 1.433 | 3494 | 0.792 | 1227 | 0.726 | 3 | rs2594935 | chr15 | 25858633 | eye color | blue vs nonblue eyes | 52 |
| 3.07E−21 | 2.184 | 3496 | 0.853 | 491 | 0.726 | 4 | rs728405 | chr15 | 25873448 | eye color | blue vs brown eyes | 104 |
| 1.66E−17 | 1.668 | 3496 | 0.853 | 1227 | 0.776 | 4 | rs728405 | chr15 | 25873448 | eye color | blue vs nonblue eyes | 104 |
| 5.22E−14 | 0.567 | 3496 | 0.221 | 491 | 0.334 | 3 | rs1448488 | chr15 | 25890452 | eye color | blue vs brown eyes | 18 |
| 1.72E−10 | 0.710 | 3496 | 0.221 | 1227 | 0.286 | 3 | rs1448488 | chr15 | 25890452 | eye color | blue vs nonblue eyes | 18 |
| 5.90E−17 | 0.457 | 3484 | 0.103 | 490 | 0.201 | 3 | rs4778220 | chr15 | 25894733 | eye color | blue vs brown eyes | 74 |
| 2.22E−11 | 0.627 | 3484 | 0.103 | 1224 | 0.155 | 3 | rs4778220 | chr15 | 25894733 | eye color | blue vs nonblue eyes | 74 |
| 4.09E−08 | 1.532 | 3482 | 0.786 | 490 | 0.706 | 3 | rs2871875 | chr15 | 25938449 | eye color | blue vs brown eyes | 54 |
| 1.37E−10 | 0.539 | 3496 | 0.105 | 490 | 0.179 | 3 | rs7170869 | chr15 | 25962343 | eye color | blue vs brown eyes | 97 |
| 7.06E−10 | 0.651 | 3496 | 0.105 | 1226 | 0.153 | 3 | rs7170869 | chr15 | 25962343 | eye color | blue vs nonblue eyes | 97 |
| 1.21E−70 | 0.070 | 3492 | 0.011 | 490 | 0.135 | 3 | rs7495174 | chr15 | 26017833 | eye color | blue vs brown eyes | 105 |
| 6.81E−50 | 0.140 | 3492 | 0.011 | 1225 | 0.072 | 3 | rs7495174 | chr15 | 26017833 | eye color | blue vs nonblue eyes | 105 |
| 1.20E−22 | 0.198 | 735 | 0.030 | 490 | 0.135 | 3 | rs7495174 | chr15 | 26017833 | eye color | green vs brown eyes | 1 |
| 1.18E−10 | 0.247 | 690 | 0.013 | 1271 | 0.051 | 3 | rs7495174 | chr15 | 26017833 | hair color | blond vs brown hair | 1 |
| 1.31E−130 | 35.284 | 3496 | 0.993 | 491 | 0.811 | 2 | rs7183877 | chr15 | 26039328 | eye color | blue vs brown eyes | |
| 2.39E−38 | 10.067 | 3496 | 0.993 | 736 | 0.938 | 2 | rs7183877 | chr15 | 26039328 | eye color | blue vs green eyes | |
| 3.48E−118 | 19.291 | 3496 | 0.993 | 1227 | 0.887 | 2 | rs7183877 | chr15 | 26039328 | eye color | blue vs nonblue eyes | |
| 6.43E−22 | 3.505 | 736 | 0.938 | 491 | 0.811 | 2 | rs7183877 | chr15 | 26039328 | eye color | green vs brown eyes | |
| 1.07E−16 | 5.801 | 691 | 0.988 | 1273 | 0.936 | 2 | rs7183877 | chr15 | 26039328 | hair color | blond vs brown hair | |
| 1.68E−67 | 52.844 | 3496 | 0.998 | 491 | 0.910 | 4 | rs8028689 | chr15 | 26162483 | eye color | blue vs brown eyes | 1 |
| 2.92E−12 | 10.031 | 3496 | 0.998 | 736 | 0.982 | 4 | rs8028689 | chr15 | 26162483 | eye color | blue vs green eyes | 1 |
| 1.39E−53 | 26.395 | 3496 | 0.998 | 1227 | 0.953 | 4 | rs8028689 | chr15 | 26162483 | eye color | blue vs nonblue eyes | 112 |
| 3.32E−16 | 5.268 | 736 | 0.982 | 491 | 0.910 | 4 | rs8028689 | chr15 | 26162483 | eye color | green vs brown eyes | 112 |
| 1.68E−67 | 0.019 | 3496 | 0.002 | 491 | 0.090 | 4 | rs2240204 | chr15 | 26167627 | eye color | blue vs brown eyes | 37 |
| 2.92E−12 | 0.100 | 3496 | 0.002 | 736 | 0.018 | 4 | rs2240204 | chr15 | 26167627 | eye color | blue vs green eyes | 37 |
| 1.39E−53 | 0.038 | 3496 | 0.002 | 1227 | 0.047 | 4 | rs2240204 | chr15 | 26167627 | eye color | blue vs nonblue eyes | 37 |
| 3.32E−16 | 0.190 | 736 | 0.018 | 491 | 0.090 | 4 | rs2240204 | chr15 | 26167627 | eye color | green vs brown eyes | 37 |
| 1.53E−181 | 26.677 | 3496 | 0.985 | 491 | 0.715 | 4 | rs8039195 | chr15 | 26189679 | eye color | blue vs brown eyes | 113 |
| 4.70E−37 | 6.044 | 3496 | 0.985 | 736 | 0.917 | 4 | rs8039195 | chr15 | 26189679 | eye color | blue vs green eyes | 113 |
| 1.37E−149 | 13.103 | 3496 | 0.985 | 1227 | 0.836 | 4 | rs8039195 | chr15 | 26189679 | eye color | blue vs nonblue eyes | 113 |
| 1.28E−39 | 4.414 | 736 | 0.917 | 491 | 0.715 | 4 | rs8039195 | chr15 | 26189679 | eye color | green vs brown eyes | 113 |
| 8.76E−22 | 4.590 | 691 | 0.976 | 1273 | 0.899 | 4 | rs8039195 | chr15 | 26189679 | hair color | blond vs brown hair | 113 |
| 1.84E−67 | 0.019 | 3492 | 0.002 | 491 | 0.090 | 3 | rs16950979 | chr15 | 26194101 | eye color | blue vs brown eyes | 26 |
| 2.83E−12 | 0.100 | 3492 | 0.002 | 734 | 0.018 | 3 | rs16950979 | chr15 | 26194101 | eye color | blue vs green eyes | 26 |
| 1.33E−53 | 0.038 | 3492 | 0.002 | 1225 | 0.047 | 3 | rs16950979 | chr15 | 26194101 | eye color | blue vs nonblue eyes | 26 |
| 3.74E−16 | 0.190 | 734 | 0.018 | 491 | 0.090 | 3 | rs16950979 | chr15 | 26194101 | eye color | green vs brown eyes | 26 |
| 1.72E−67 | 52.829 | 3495 | 0.998 | 491 | 0.910 | 3 | rs16950987 | chr15 | 26199823 | eye color | blue vs brown eyes | 27 |
| 2.94E−12 | 10.028 | 3495 | 0.998 | 736 | 0.982 | 3 | rs16950987 | chr15 | 26199823 | eye color | blue vs green eyes | 27 |
| 1.42E−53 | 26.387 | 3495 | 0.998 | 1227 | 0.953 | 3 | rs16950987 | chr15 | 26199823 | eye color | blue vs nonblue eyes | 27 |
| 3.32E−16 | 5.268 | 736 | 0.982 | 491 | 0.910 | 3 | rs16950987 | chr15 | 26199823 | eye color | green vs brown eyes | 27 |
| 1.89E−219 | 0.029 | 3494 | 0.015 | 490 | 0.348 | 3 | rs1667394 | chr15 | 26203777 | eye color | blue vs brown eyes | 25 |
| 1.06E−43 | 0.153 | 3494 | 0.015 | 735 | 0.093 | 3 | rs1667394 | chr15 | 26203777 | eye color | blue vs green eyes | 25 |
| 9.32E−189 | 0.065 | 3494 | 0.015 | 1225 | 0.195 | 3 | rs1667394 | chr15 | 26203777 | eye color | blue vs nonblue eyes | 25 |
| 2.63E−54 | 0.193 | 735 | 0.093 | 490 | 0.348 | 3 | rs1667394 | chr15 | 26203777 | eye color | green vs brown eyes | 25 |
| 5.61E−29 | 0.187 | 691 | 0.025 | 1271 | 0.122 | 3 | rs1667394 | chr15 | 26203777 | hair color | blond vs brown hair | 25 |
| 9.41E−08 | 1.849 | 3468 | 0.931 | 486 | 0.880 | 4 | rs1907001 | chr15 | 27053851 | eye color | blue vs brown eyes | |
| 5.94E−08 | 1.874 | 3433 | 0.932 | 484 | 0.880 | 4 | rs7165740 | chr15 | 27057792 | eye color | blue vs brown eyes | |
| 9.59E−09 | 1.971 | 3377 | 0.936 | 476 | 0.881 | 4 | rs12441723 | chr15 | 27120318 | eye color | blue vs brown eyes | |

C. Variants associated with pigmentation on chromosomes 6, 12 and 14

| | | | | | | | | | | | | |
|---|---|---|---|---|---|---|---|---|---|---|---|---|
| 6.91E−08 | 0.714 | 2407 | 0.110 | 2204 | 0.148 | 3 | rs1050975 | chr6 | 353012 | freckles | freckles vs non-freckles | 6 |
| 9.30E−09 | 1.272 | 2405 | 0.481 | 2204 | 0.422 | 3 | rs872071 | chr6 | 356064 | freckles | freckles vs non-freckles | 119 |
| 4.19E−08 | 1.270 | 2403 | 0.669 | 2203 | 0.614 | 3 | rs7757906 | chr6 | 357741 | freckles | freckles vs non-freckles | 110 |
| 2.41E−09 | 1.356 | 2407 | 0.812 | 2204 | 0.761 | 3 | rs11242867 | chr6 | 360406 | freckles | freckles vs non-freckles | 10 |
| 6.99E−08 | 1.442 | 1145 | 0.821 | 1584 | 0.761 | 3 | rs9503644 | chr6 | 360406 | skin sun sensitivity | freckles/sun sensitive vs | 10 |
| | | | | | | | | | | | non-freckles/not sun | |
| | | | | | | | | | | | sensitive | |
| 8.95E−12 | 1.334 | 2406 | 0.463 | 2203 | 0.393 | 2 | rs9378805 | chr6 | 362727 | freckles | freckles vs non-freckles | 125 |
| 6.32E−09 | 1.380 | 1145 | 0.475 | 1583 | 0.396 | 2 | rs9378805 | chr6 | 362727 | skin sun sensitivity | freckles/sun sensitive vs | 125 |
| | | | | | | | | | | | non-freckles/not sun | |
| | | | | | | | | | | | sensitive | |
| 9.39E−08 | 0.737 | 2406 | 0.138 | 2204 | 0.179 | 4 | rs950286 | chr6 | 374457 | freckles | freckles vs non-freckles | 129 |
| 9.39E−12 | 1.329 | 2407 | 0.518 | 2204 | 0.447 | 3 | rs9328192 | chr6 | 379364 | freckles | freckles vs non-freckles | 124 |
| 2.04E−09 | 1.390 | 1145 | 0.531 | 1584 | 0.449 | 3 | rs9328192 | chr6 | 379364 | skin sun sensitivity | freckles/sun sensitive vs | 124 |
| | | | | | | | | | | | non-freckles/not sun | |
| | | | | | | | | | | | sensitive | |
| 1.92E−08 | 0.786 | 2406 | 0.353 | 2204 | 0.410 | 3 | rs9405675 | chr6 | 389600 | freckles | freckles vs non-freckles | 126 |
| 8.57E−10 | 0.754 | 2406 | 0.260 | 2202 | 0.318 | 4 | rs9405681 | chr6 | 394358 | freckles | freckles vs non-freckles | 127 |
| 3.69E−09 | 0.698 | 1145 | 0.249 | 1582 | 0.322 | 4 | rs9405681 | chr6 | 394358 | skin sun sensitivity | freckles/sun sensitive vs | 127 |
| | | | | | | | | | | | non-freckles/not sun | |
| | | | | | | | | | | | sensitive | |
| 6.11E−16 | 0.712 | 2407 | 0.502 | 2204 | 0.586 | 2 | rs4959270 | chr6 | 402748 | freckles | freckles vs non-freckles | 87 |
| 1.67E−14 | 0.655 | 1145 | 0.480 | 1584 | 0.585 | 2 | rs4959270 | chr6 | 402748 | skin sun sensitivity | freckles/sun sensitive vs | 87 |
| | | | | | | | | | | | non-freckles/not sun | |
| | | | | | | | | | | | sensitive | |
| 4.24E−16 | 0.711 | 2403 | 0.494 | 2197 | 0.579 | 3 | rs1540771 | chr6 | 411033 | freckles | freckles vs non-freckles | 21 |
| 4.66E−09 | 1.483 | 688 | 0.583 | 1271 | 0.485 | 3 | rs1540771 | chr6 | 411033 | hair color | blond vs brown hair | 21 |
| 5.36E−13 | 0.672 | 1143 | 0.477 | 1580 | 0.576 | 3 | rs1540771 | chr6 | 411033 | skin sun sensitivity | freckles/sun sensitive vs | 21 |
| | | | | | | | | | | | non-freckles/not sun | |
| | | | | | | | | | | | sensitive | |
| 5.42E−08 | 1.273 | 2405 | 0.696 | 2203 | 0.643 | 4 | rs950039 | chr6 | 438976 | freckles | freckles vs non-freckles | 128 |
| 5.79E−08 | 1.704 | 690 | 0.891 | 1273 | 0.828 | 4 | rs4842602 | chr12 | 87235053 | hair color | blond vs brown hair | 82 |
| 1.98E−08 | 1.726 | 691 | 0.889 | 1272 | 0.823 | 3 | rs995030 | chr12 | 87393139 | hair color | blond vs brown hair | 134 |
| 1.88E−08 | 1.730 | 690 | 0.890 | 1270 | 0.824 | 2 | rs1022034 | chr12 | 87421211 | hair color | blond vs brown hair | 2 |
| 9.21E−08 | 0.606 | 691 | 0.123 | 1271 | 0.188 | 2 | rs3782181 | chr12 | 87456029 | hair color | blond vs brown hair | 62 |
| 1.95E−24 | 0.436 | 690 | 0.710 | 1271 | 0.849 | 4 | rs12821256 | chr12 | 87830803 | hair color | blond vs brown hair | 15 |
| 1.28E−08 | 1.617 | 3494 | 0.897 | 736 | 0.844 | 3 | rs8016079 | chr14 | 91828198 | eye color | blue vs green eyes | 111 |
| 2.21E−12 | 1.544 | 3489 | 0.738 | 734 | 0.646 | 3 | rs4904864 | chr14 | 91834272 | eye color | blue vs green eyes | 83 |
| 1.89E−12 | 1.434 | 3489 | 0.738 | 1225 | 0.663 | 3 | rs4904864 | chr14 | 91834272 | eye color | blue vs nonblue eyes | 83 |
| 4.76E−18 | 1.939 | 689 | 0.792 | 1270 | 0.662 | 3 | rs4904864 | chr14 | 91834272 | hair color | blond vs brown hair | 83 |
| 6.98E−20 | 0.585 | 3495 | 0.325 | 735 | 0.452 | 4 | rs4904868 | chr14 | 91850754 | eye color | blue vs green eyes | 84 |
| 5.70E−17 | 0.666 | 3495 | 0.325 | 1226 | 0.420 | 4 | rs4904868 | chr14 | 91850754 | eye color | blue vs nonblue eyes | 84 |
| 1.08E−22 | 0.495 | 690 | 0.260 | 1272 | 0.415 | 4 | rs4904868 | chr14 | 91850754 | hair color | blond vs brown hair | 84 |
| 3.24E−09 | 0.649 | 3495 | 0.153 | 736 | 0.218 | 3 | rs2402130 | chr14 | 91870956 | eye color | blue vs green eyes | 49 |
| 1.51E−15 | 0.463 | 691 | 0.103 | 1273 | 0.200 | 3 | rs2402130 | chr14 | 91870956 | hair color | blond vs brown hair | 49 |

D. Variants associated with pigmentation on chromosomes 1, 4, 9, 11, 18 and 20

| | | | | | | | | | | | | |
|---|---|---|---|---|---|---|---|---|---|---|---|---|
| 4.47E−08 | 0.655 | 3492 | 0.082 | 1227 | 0.121 | 2 | rs630446 | chr1 | 55662008 | eye color | blue vs nonblue eyes | 93 |
| 5.31E−08 | 0.568 | 3495 | 0.060 | 736 | 0.101 | 4 | rs11206611 | chr1 | 55679165 | eye color | blue vs green eyes | 9 |
| 4.70E−09 | 0.601 | 3495 | 0.060 | 1227 | 0.096 | 4 | rs11206611 | chr1 | 55679165 | eye color | blue vs nonblue eyes | 9 |
| 2.89E−08 | 1.509 | 3493 | 0.362 | 491 | 0.273 | 2 | rs7684457 | chr4 | 101882168 | eye color | blue vs brown eyes | 109 |
| 4.22E−08 | 1.503 | 3493 | 0.358 | 491 | 0.271 | 3 | rs7680366 | chr4 | 101929217 | eye color | blue vs brown eyes | 108 |
| 1.84E−08 | 0.707 | 3490 | 0.149 | 1225 | 0.198 | 3 | rs1022901 | chr9 | 12578259 | eye color | blue vs nonblue eyes | 3 |
| 1.08E−08 | 0.748 | 3495 | 0.276 | 1227 | 0.338 | 4 | rs10809808 | chr9 | 12614463 | eye color | blue vs nonblue eyes | 7 |
| 1.89E−08 | 0.695 | 3494 | 0.227 | 736 | 0.298 | 4 | rs1408799 | chr9 | 12662097 | eye color | blue vs green eyes | 17 |
| 1.49E−12 | 0.687 | 3494 | 0.227 | 1227 | 0.300 | 4 | rs1408799 | chr9 | 12662097 | eye color | blue vs nonblue eyes | 17 |
| 8.60E−08 | 1.302 | 3490 | 0.684 | 1225 | 0.624 | 3 | rs927869 | chr9 | 12738962 | eye color | blue vs nonblue eyes | 123 |
| 1.25E−08 | 0.632 | 690 | 0.189 | 1271 | 0.269 | 4 | rs896978 | chr11 | 68585505 | hair color | blond vs brown hair | 122 |
| 1.53E−09 | 0.620 | 691 | 0.198 | 1272 | 0.284 | 3 | rs3750965 | chr11 | 68596736 | hair color | blond vs brown hair | 59 |
| 7.56E−08 | 1.563 | 684 | 0.242 | 1259 | 0.170 | 4 | rs2305498 | chr11 | 68623490 | hair color | blond vs brown hair | 43 |
| 7.91E−10 | 0.650 | 690 | 0.312 | 1273 | 0.410 | 3 | rs1011176 | chr11 | 68690473 | hair color | blond vs brown hair | 1 |
| 2.96E−09 | 1.311 | 2407 | 0.730 | 2203 | 0.674 | 2 | rs1042602 | chr11 | 88551344 | freckles | freckles vs non-freckles | 4 |
| 9.35E−11 | 0.654 | 3494 | 0.689 | 736 | 0.772 | 3 | rs1393350 | chr11 | 88650694 | eye color | blue vs green eyes | 16 |
| 3.42E−09 | 0.732 | 3494 | 0.689 | 1227 | 0.752 | 3 | rs1393350 | chr11 | 88650694 | eye color | blue vs nonblue eyes | 16 |
| 4.20E−08 | 0.770 | 1675 | 0.673 | 2821 | 0.728 | 3 | rs1393350 | chr11 | 88650694 | skin sun sensitivity | burner vs tanner | 16 |
| 9.80E−08 | 0.684 | 3495 | 0.590 | 491 | 0.678 | 4 | rs4453582 | chr18 | 34735189 | eye color | blue vs brown eyes | 69 |
| 1.37E−08 | 0.723 | 1143 | 0.596 | 1581 | 0.671 | 2 | rs4911379 | chr20 | 31998966 | skin sun sensitivity | freckles/sun sensitive vs | 85 |
| | | | | | | | | | | | non-freckles/not sun | |
| | | | | | | | | | | | sensitive | |
| 3.90E−08 | 1.370 | 1145 | 0.395 | 1584 | 0.323 | 4 | rs2284378 | chr20 | 32051756 | skin sun sensitivity | freckles/sun sensitive vs | 42 |
| | | | | | | | | | | | non-freckles/not sun | |
| | | | | | | | | | | | sensitive | |
| 6.41E−08 | 1.363 | 1135 | 0.400 | 1571 | 0.328 | 4 | rs4911414 | chr20 | 32193105 | skin sun sensitivity | freckles/sun sensitive vs | 86 |
| | | | | | | | | | | | non-freckles/not sun | |
| | | | | | | | | | | | sensitive | |
| 1.61E−08 | 1.364 | 1145 | 0.559 | 1584 | 0.482 | 4 | rs2225837 | chr20 | 32469295 | skin sun sensitivity | freckles/sun sensitive vs | 35 |
| | | | | | | | | | | | non-freckles/not sun | |
| | | | | | | | | | | | sensitive | |
| 1.84E−08 | 1.363 | 1145 | 0.559 | 1583 | 0.482 | 3 | rs6120650 | chr20 | 32503634 | skin sun sensitivity | freckles/sun sensitive vs | 91 |
| | | | | | | | | | | | non-freckles/not sun | |
| | | | | | | | | | | | sensitive | |
| 5.35E−10 | 1.506 | 1144 | 0.259 | 1581 | 0.188 | 4 | rs2281695 | chr20 | 32592825 | skin sun sensitivity | freckles/sun sensitive vs | 41 |
| | | | | | | | | | | | non-freckles/not sun | |
| | | | | | | | | | | | sensitive | |
| 7.02E−08 | 1.354 | 1103 | 0.577 | 1524 | 0.502 | 2 | rs6059909 | chr20 | 32603352 | skin sun sensitivity | freckles/sun sensitive vs | 88 |
| | | | | | | | | | | | non-freckles/not sun | |
| | | | | | | | | | | | sensitive | |
| 7.76E−08 | 1.364 | 2406 | 0.176 | 2203 | 0.135 | 4 | rs2378199 | chr20 | 32650141 | skin sun sensitivity | freckles vs non-freckles | 47 |
| 5.12E−11 | 1.629 | 1144 | 0.198 | 1584 | 0.132 | 4 | rs2378199 | chr20 | 32650141 | skin sun sensitivity | freckles/sun sensitive vs | 47 |
| | | | | | | | | | | | non-freckles/not sun | |
| | | | | | | | | | | | sensitive | |
| 3.76E−11 | 1.633 | 1145 | 0.199 | 1582 | 0.132 | 3 | rs2378249 | chr20 | 32681751 | skin sun sensitivity | freckles/sun sensitive vs | 48 |
| | | | | | | | | | | | non-freckles/not sun | |
| | | | | | | | | | | | sensitive | |
| 6.04E−11 | 1.622 | 1145 | 0.200 | 1584 | 0.133 | 4 | rs6060034 | chr20 | 32815525 | skin sun sensitivity | freckles/sun sensitive vs | 89 |
| | | | | | | | | | | | non-freckles/not sun | |
| | | | | | | | | | | | sensitive | |
| 5.33E−11 | 0.615 | 1143 | 0.800 | 1583 | 0.867 | 4 | rs6060043 | chr20 | 32828245 | skin sun sensitivity | freckles/sun sensitive vs | 90 |
| | | | | | | | | | | | non-freckles/not sun | |
| | | | | | | | | | | | sensitive | |
| 1.54E−09 | 0.621 | 1145 | 0.829 | 1583 | 0.886 | 3 | rs619865 | chr20 | 33331111 | skin sun sensitivity | freckles/sun sensitive vs | 92 |
| | | | | | | | | | | | non-freckles/not sun | |
| | | | | | | | | | | | sensitive | |

*Comparison is based pigmentation phenotypes as defined above. Burner vs tanner refers to skin sensitive vs non skin sensitive comparison, and freckles/sun sensitive vs non-freckles/non sun sensitive refers to those who fulfill both criteria (i.e., either have freckles and are sun sensitive or do not have freckles and are not sun sensitive, based on the Fitzpatrick scale).

TABLE 11
Markers in linkage disequilibrium with the markers listed in Table 10. All markers in
the HapMap CEU data that are in LD with at least one of the markers in Table 10
with a value for r² of greater than 0.2 are listed. Shown are the associated marker,
the marker from Table 10 to which the LD is strongest, as well as values for the LD
measures r² and D′, and the p-value for the observed LD.

| | | | | | | Position |
|---|---|---|---|---|---|---|
| Correlated | | | | | | NCBI Build |
| SNP | Anchor SNP | D′ | R2 | P-VALUE | Chromosome | 35 |
| rs7534376 | rs11206611 | 0.732143 | 0.258775 | 0.000301 | Chr1 | 55565404 |
| rs11206580 | rs11206611 | 1 | 0.237288 | 0.000756 | Chr1 | 55664387 |
| rs12022663 | rs630446 | 0.747102 | 0.3742 | 3.65E−07 | Chr1 | 55676891 |
| rs12024547 | rs11206611 | 1 | 0.237288 | 0.000756 | Chr1 | 55687617 |
| rs12566719 | rs630446 | 0.719626 | 0.304457 | 4.70E−06 | Chr1 | 55689357 |
| rs11206586 | rs630446 | 0.813084 | 0.28639 | 0.000013 | Chr1 | 55689508 |
| rs12066898 | rs630446 | 0.813084 | 0.28639 | 0.000013 | Chr1 | 55701625 |
| rs1499680 | rs11206611 | 1 | 0.237288 | 0.000756 | Chr1 | 55710252 |
| rs7555620 | rs630446 | 0.810127 | 0.285261 | 0.000014 | Chr1 | 55711377 |
| rs7532502 | rs630446 | 0.813084 | 0.28639 | 0.000013 | Chr1 | 55711475 |
| rs12564538 | rs630446 | 0.811617 | 0.28583 | 0.000014 | Chr1 | 55712441 |
| rs10493199 | rs11206611 | 1 | 0.236842 | 0.000814 | Chr1 | 55712928 |
| rs10493198 | rs630446 | 0.813084 | 0.28639 | 0.000013 | Chr1 | 55713103 |
| rs1740127 | rs11206611 | 1 | 0.318182 | 4.45E−08 | Chr1 | 55714584 |
| rs356088 | rs630446 | 1 | 0.736119 | 1.40E−15 | Chr1 | 55717159 |
| rs356087 | rs630446 | 1 | 1 | 1.96E−17 | Chr1 | 55717949 |
| rs769894 | rs630446 | 1 | 1 | 1.16E−19 | Chr1 | 55721565 |
| rs630446 | rs630446 | 1 | 1 | 0 | Chr1 | 55722575 |
| rs370904 | rs630446 | 1 | 1 | 1.16E−19 | Chr1 | 55724093 |
| rs390026 | rs11206611 | 1 | 1 | 2.03E−14 | Chr1 | 55725693 |
| rs12043386 | rs11206611 | 1 | 1 | 1.76E−14 | Chr1 | 55725992 |
| rs379213 | rs11206611 | 1 | 0.642857 | 4.22E−11 | Chr1 | 55727215 |
| rs412115 | rs11206611 | 1 | 0.642857 | 4.46E−10 | Chr1 | 55727504 |
| rs380389 | rs11206611 | 1 | 1 | 1.89E−14 | Chr1 | 55729860 |
| rs396511 | rs11206611 | 1 | 1 | 3.18E−13 | Chr1 | 55730691 |
| rs1114737 | rs630446 | 0.893903 | 0.291247 | 1.07E−07 | Chr1 | 55738001 |
| rs11206611 | rs11206611 | 1 | 1 | 0 | Chr1 | 55739732 |
| rs1780522 | rs630446 | 0.893903 | 0.291247 | 1.07E−07 | Chr1 | 55740877 |
| rs1780521 | rs630446 | 0.893903 | 0.291247 | 1.07E−07 | Chr1 | 55740986 |
| rs1695938 | rs630446 | 0.893903 | 0.291247 | 1.07E−07 | Chr1 | 55741038 |
| rs17416336 | rs11206611 | 1 | 1 | 1.76E−14 | Chr1 | 55742336 |
| rs1780519 | rs630446 | 0.893903 | 0.291247 | 1.07E−07 | Chr1 | 55742855 |
| rs356118 | rs630446 | 1 | 0.91453 | 3.63E−17 | Chr1 | 55744847 |
| rs356119 | rs630446 | 1 | 0.907479 | 3.47E−16 | Chr1 | 55745088 |
| rs1695941 | rs630446 | 0.899295 | 0.355237 | 1.11E−08 | Chr1 | 55746074 |
| rs1542856 | rs630446 | 0.901793 | 0.395215 | 2.97E−09 | Chr1 | 55755350 |
| rs1695961 | rs630446 | 1 | 0.476534 | 2.30E−09 | Chr1 | 55756189 |
| rs410923 | rs630446 | 0.616643 | 0.217793 | 0.000027 | Chr1 | 55758464 |
| rs424713 | rs11206611 | 1 | 1 | 1.76E−14 | Chr1 | 55760086 |
| rs1780541 | rs630446 | 0.901793 | 0.395215 | 2.97E−09 | Chr1 | 55760382 |
| rs1780540 | rs630446 | 0.901793 | 0.395215 | 2.97E−09 | Chr1 | 55760394 |
| rs904610 | rs630446 | 0.902996 | 0.417807 | 1.44E−09 | Chr1 | 55762002 |
| rs11206616 | rs11206611 | 1 | 1 | 1.76E−14 | Chr1 | 55763224 |
| rs1321120 | rs11206611 | 1 | 0.785714 | 2.87E−12 | Chr1 | 55765398 |
| rs7529841 | rs11206611 | 1 | 0.774436 | 4.71E−12 | Chr1 | 55768057 |
| rs168549 | rs630446 | 0.90056 | 0.374428 | 5.85E−09 | Chr1 | 55771023 |
| rs4083594 | rs11206611 | 0.639611 | 0.301443 | 0.0001 | Chr1 | 55944645 |
| rs6830710 | rs7684457 | 0.775514 | 0.273046 | 4.66E−07 | Chr4 | 101518581 |
| rs1991843 | rs7684457 | 0.781295 | 0.276874 | 1.95E−07 | Chr4 | 101524916 |
| rs7688363 | rs7684457 | 0.801889 | 0.316668 | 6.91E−09 | Chr4 | 101549012 |
| rs17552895 | rs7684457 | 0.801889 | 0.316668 | 6.91E−09 | Chr4 | 101549610 |
| rs10516464 | rs7684457 | 0.801889 | 0.316668 | 6.91E−09 | Chr4 | 101552381 |
| rs930236 | rs7684457 | 0.795368 | 0.312764 | 1.57E−08 | Chr4 | 101561108 |
| rs1501106 | rs7684457 | 0.542033 | 0.27323 | 3.57E−07 | Chr4 | 101571370 |
| rs357652 | rs7684457 | 0.570184 | 0.27812 | 3.70E−07 | Chr4 | 101572145 |
| rs6838262 | rs7684457 | 0.449613 | 0.202152 | 8.31E−06 | Chr4 | 101577666 |
| rs1114130 | rs7684457 | 0.449613 | 0.202152 | 8.31E−06 | Chr4 | 101582539 |
| rs357669 | rs7684457 | 0.502431 | 0.208322 | 2.42E−06 | Chr4 | 101592750 |
| rs768822 | rs7684457 | 0.475774 | 0.217867 | 3.53E−06 | Chr4 | 101601637 |
| rs4699802 | rs7684457 | 0.475774 | 0.217867 | 3.53E−06 | Chr4 | 101601862 |
| rs1501109 | rs7684457 | 0.475774 | 0.217867 | 3.53E−06 | Chr4 | 101603514 |
| rs4699402 | rs7684457 | 0.475774 | 0.217867 | 3.53E−06 | Chr4 | 101603990 |
| rs11097720 | rs7684457 | 0.475774 | 0.217867 | 3.53E−06 | Chr4 | 101607171 |
| rs924534 | rs7684457 | 0.710978 | 0.305749 | 4.09E−08 | Chr4 | 101616867 |
| rs924531 | rs7684457 | 0.716991 | 0.309977 | 1.74E−08 | Chr4 | 101617228 |
| rs736517 | rs7684457 | 0.475774 | 0.217867 | 3.53E−06 | Chr4 | 101618062 |
| rs3775364 | rs7684457 | 0.711737 | 0.298888 | 4.46E−08 | Chr4 | 101645912 |
| rs3775365 | rs7684457 | 0.504024 | 0.214483 | 0.000012 | Chr4 | 101647277 |
| rs6820177 | rs7684457 | 0.449613 | 0.202152 | 8.31E−06 | Chr4 | 101648476 |
| rs7691351 | rs7684457 | 0.699017 | 0.274137 | 1.97E−07 | Chr4 | 101649322 |
| rs11935753 | rs7684457 | 0.699017 | 0.274137 | 1.97E−07 | Chr4 | 101652358 |
| rs6828794 | rs7684457 | 0.475751 | 0.216854 | 0.00001 | Chr4 | 101653387 |
| rs992279 | rs7684457 | 0.738728 | 0.294167 | 8.93E−07 | Chr4 | 101655341 |
| rs11945725 | rs7684457 | 0.652427 | 0.258433 | 1.00E−06 | Chr4 | 101658586 |
| rs1846870 | rs7684457 | 0.610912 | 0.219628 | 1.96E−06 | Chr4 | 101673196 |
| rs6822114 | rs7684457 | 0.601363 | 0.214398 | 3.50E−06 | Chr4 | 101673556 |
| rs6816158 | rs7684457 | 0.599934 | 0.251101 | 4.67E−07 | Chr4 | 101674654 |
| rs1501084 | rs7684457 | 1 | 0.290963 | 4.15E−09 | Chr4 | 101682119 |
| rs1846869 | rs7684457 | 0.79137 | 0.557447 | 4.87E−15 | Chr4 | 101682200 |
| rs1392874 | rs7684457 | 0.751087 | 0.469364 | 5.98E−13 | Chr4 | 101685843 |
| rs10019286 | rs7684457 | 0.9537 | 0.686928 | 7.13E−20 | Chr4 | 101688567 |
| rs10029873 | rs7684457 | 0.778018 | 0.518224 | 1.55E−13 | Chr4 | 101688842 |
| rs977668 | rs7684457 | 0.816178 | 0.534379 | 1.31E−13 | Chr4 | 101689254 |
| rs11728780 | rs7684457 | 0.948078 | 0.623363 | 4.27E−17 | Chr4 | 101689680 |
| rs7682797 | rs7684457 | 0.924903 | 0.362435 | 3.05E−10 | Chr4 | 101690536 |
| rs2651545 | rs7680366 | 1 | 0.224267 | 1.41E−07 | Chr4 | 101694759 |
| rs2651546 | rs7684457 | 0.908213 | 0.645415 | 2.15E−16 | Chr4 | 101696643 |
| rs2134004 | rs7684457 | 0.620107 | 0.304263 | 4.27E−08 | Chr4 | 101697060 |
| rs1392876 | rs7684457 | 0.835105 | 0.579992 | 2.40E−16 | Chr4 | 101698146 |
| rs7655358 | rs7684457 | 0.587107 | 0.29199 | 4.47E−08 | Chr4 | 101699089 |
| rs7685369 | rs7684457 | 0.570388 | 0.255384 | 2.26E−07 | Chr4 | 101699609 |
| rs7667609 | rs7684457 | 0.58571 | 0.27026 | 7.30E−08 | Chr4 | 101700564 |
| rs1846877 | rs7684457 | 0.875178 | 0.61464 | 1.38E−17 | Chr4 | 101705317 |
| rs2651581 | rs7684457 | 0.913357 | 0.644799 | 2.02E−18 | Chr4 | 101705912 |
| rs2651578 | rs7680366 | 1 | 0.239766 | 3.90E−08 | Chr4 | 101709306 |
| rs2651576 | rs7680366 | 1 | 0.239766 | 3.90E−08 | Chr4 | 101709830 |
| rs2567396 | rs7680366 | 1 | 0.224267 | 1.03E−07 | Chr4 | 101709860 |
| rs1846874 | rs7684457 | 0.915949 | 0.201972 | 3.57E−07 | Chr4 | 101711803 |
| rs6815548 | rs7684457 | 1 | 0.492466 | 9.81E−16 | Chr4 | 101711928 |
| rs12507347 | rs7684457 | 0.915949 | 0.201972 | 3.57E−07 | Chr4 | 101713125 |
| rs11936939 | rs7684457 | 0.921772 | 0.788261 | 3.84E−23 | Chr4 | 101713483 |
| rs2651574 | rs7684457 | 0.913192 | 0.200449 | 8.00E−07 | Chr4 | 101714414 |
| rs12499640 | rs7684457 | 0.915949 | 0.201972 | 3.57E−07 | Chr4 | 101714813 |
| rs2651587 | rs7680366 | 1 | 0.239766 | 3.13E−08 | Chr4 | 101717011 |
| rs1501112 | rs7684457 | 0.915216 | 0.646187 | 2.65E−18 | Chr4 | 101718834 |
| rs2567388 | rs7680366 | 1 | 0.331984 | 7.02E−11 | Chr4 | 101720784 |
| rs6848407 | rs7684457 | 1 | 0.390708 | 1.01E−12 | Chr4 | 101722300 |
| rs17030283 | rs7684457 | 0.927753 | 0.379201 | 3.72E−11 | Chr4 | 101724191 |
| rs2567381 | rs7684457 | 0.915835 | 0.67246 | 2.09E−19 | Chr4 | 101725537 |
| rs2651562 | rs7680366 | 1 | 0.210978 | 7.29E−07 | Chr4 | 101725818 |
| rs1501103 | rs7684457 | 0.909061 | 0.649391 | 3.70E−17 | Chr4 | 101726003 |
| rs2651563 | rs7680366 | 0.874317 | 0.202471 | 0.000011 | Chr4 | 101726562 |
| rs2567380 | rs7684457 | 0.916961 | 0.67473 | 1.05E−19 | Chr4 | 101726608 |
| rs2567379 | rs7684457 | 0.916961 | 0.67473 | 1.05E−19 | Chr4 | 101726844 |
| rs2651566 | rs7684457 | 0.916748 | 0.691653 | 2.22E−19 | Chr4 | 101726991 |
| rs6816732 | rs7684457 | 0.91754 | 0.234026 | 2.28E−07 | Chr4 | 101727543 |
| rs2651569 | rs7684457 | 0.939273 | 0.359429 | 4.10E−11 | Chr4 | 101727645 |
| rs2651570 | rs7684457 | 0.959328 | 0.822211 | 3.18E−24 | Chr4 | 101727750 |
| rs17632841 | rs7684457 | 1 | 0.30566 | 3.87E−10 | Chr4 | 101728115 |
| rs2567375 | rs7684457 | 1 | 0.745223 | 3.18E−25 | Chr4 | 101728231 |
| rs17030316 | rs7684457 | 0.797272 | 0.440061 | 4.03E−12 | Chr4 | 101728415 |
| rs1501104 | rs7684457 | 1 | 0.747833 | 2.14E−25 | Chr4 | 101729077 |
| rs6830402 | rs7684457 | 0.601971 | 0.297544 | 8.63E−09 | Chr4 | 101729560 |
| rs6831462 | rs7684457 | 0.670863 | 0.339903 | 1.41E−09 | Chr4 | 101730107 |
| rs2567373 | rs7680366 | 1 | 0.262174 | 7.13E−09 | Chr4 | 101730131 |
| rs17030321 | rs7684457 | 0.799422 | 0.314723 | 1.36E−08 | Chr4 | 101730918 |
| rs2651573 | rs7684457 | 1 | 0.89418 | 4.71E−30 | Chr4 | 101731287 |
| rs17030327 | rs7684457 | 0.681377 | 0.365755 | 2.69E−10 | Chr4 | 101731898 |
| rs12506798 | rs7684457 | 1 | 0.240741 | 1.98E−10 | Chr4 | 101732308 |
| rs2567372 | rs7684457 | 1 | 0.774557 | 3.39E−26 | Chr4 | 101733466 |
| rs6857393 | rs7684457 | 1 | 0.415385 | 1.91E−13 | Chr4 | 101733610 |
| rs7671093 | rs7684457 | 1 | 0.89418 | 4.71E−30 | Chr4 | 101734476 |
| rs6854654 | rs7684457 | 1 | 0.231824 | 3.90E−10 | Chr4 | 101734993 |
| rs13124897 | rs7684457 | 1 | 0.828809 | 1.80E−27 | Chr4 | 101735614 |
| rs6812837 | rs7680366 | 1 | 0.262174 | 7.13E−09 | Chr4 | 101735858 |
| rs6841705 | rs7684457 | 1 | 0.231824 | 3.90E−10 | Chr4 | 101736548 |
| rs13106411 | rs7684457 | 1 | 0.891697 | 1.04E−29 | Chr4 | 101737213 |
| rs12163676 | rs7680366 | 1 | 0.262174 | 7.13E−09 | Chr4 | 101738275 |
| rs2866217 | rs7684457 | 1 | 0.231824 | 3.90E−10 | Chr4 | 101742335 |
| rs2866218 | rs7684457 | 1 | 1 | 8.83E−35 | Chr4 | 101742356 |
| rs7655291 | rs7684457 | 1 | 0.227231 | 5.85E−10 | Chr4 | 101742679 |
| rs7684457 | rs7684457 | 1 | 1 | 0 | Chr4 | 101744013 |
| rs7684866 | rs7684457 | 1 | 1 | 6.39E−34 | Chr4 | 101744280 |
| rs2866223 | rs7680366 | 1 | 0.405941 | 5.07E−13 | Chr4 | 101744971 |
| rs6838945 | rs7684457 | 1 | 1 | 1.87E−33 | Chr4 | 101745259 |
| rs4699804 | rs7684457 | 1 | 0.259259 | 4.92E−11 | Chr4 | 101746358 |
| rs7669003 | rs7684457 | 1 | 0.231824 | 3.90E−10 | Chr4 | 101146879 |
| rs2866224 | rs7684457 | 1 | 1 | 1.87E−33 | Chr4 | 101747230 |
| rs7660535 | rs7680366 | 1 | 0.405941 | 6.05E−13 | Chr4 | 101748939 |
| rs9683590 | rs7684457 | 1 | 0.743119 | 7.93E−23 | Chr4 | 101749403 |
| rs10006468 | rs7680366 | 1 | 0.262174 | 7.13E−09 | Chr4 | 101753482 |
| rs12644057 | rs7680366 | 1 | 0.405941 | 5.07E−13 | Chr4 | 101754001 |
| rs7670657 | rs7680366 | 1 | 0.262174 | 7.13E−09 | Chr4 | 101755330 |
| rs7699643 | rs7680366 | 1 | 0.262174 | 7.13E−09 | Chr4 | 101756747 |
| rs7699888 | rs7680366 | 1 | 0.262174 | 7.13E−09 | Chr4 | 101756889 |
| rs7700078 | rs7680366 | 1 | 0.262174 | 7.13E−09 | Chr4 | 101756964 |
| rs7653963 | rs7680366 | 1 | 0.262174 | 7.13E−09 | Chr4 | 101757162 |
| rs7685979 | rs7684457 | 0.925943 | 0.378005 | 1.82E−10 | Chr4 | 101761423 |
| rs2866227 | rs7680366 | 1 | 0.392971 | 3.86E−12 | Chr4 | 101762583 |
| rs2866228 | rs7684457 | 1 | 0.231824 | 3.90E−10 | Chr4 | 101763848 |
| rs2903221 | rs7680366 | 1 | 0.262174 | 9.02E−09 | Chr4 | 101768256 |
| rs2866231 | rs7684457 | 1 | 0.208294 | 4.42E−09 | Chr4 | 101768276 |
| rs13126667 | rs7680366 | 1 | 0.207985 | 7.25E−09 | Chr4 | 101769630 |
| rs7658792 | rs7680366 | 1 | 0.262174 | 7.13E−09 | Chr4 | 101770239 |
| rs2866232 | rs7680366 | 1 | 0.262174 | 1.02E−08 | Chr4 | 101770684 |
| rs7694836 | rs7684457 | 1 | 0.231824 | 3.90E−10 | Chr4 | 101771571 |
| rs6834154 | rs7680366 | 1 | 0.405941 | 5.07E−13 | Chr4 | 101772544 |
| rs7681405 | rs7680366 | 1 | 0.405941 | 5.07E−13 | Chr4 | 101778363 |
| rs10025439 | rs7680366 | 1 | 0.262174 | 7.13E−09 | Chr4 | 101788131 |
| rs7680366 | rs7680366 | 1 | 1 | 0 | Chr4 | 101791062 |
| rs7665212 | rs7680366 | 0.892855 | 0.227203 | 9.12E−07 | Chr4 | 101792259 |
| rs9759759 | rs7680366 | 0.892855 | 0.227203 | 9.12E−07 | Chr4 | 101792711 |
| rs13118526 | rs7680366 | 1 | 0.231707 | 3.95E−10 | Chr4 | 101798363 |
| rs7687955 | rs7680366 | 0.892855 | 0.227203 | 9.12E−07 | Chr4 | 101800433 |
| rs7670172 | rs7680366 | 0.892855 | 0.227203 | 9.12E−07 | Chr4 | 101801702 |
| rs12506110 | rs7680366 | 0.959832 | 0.821123 | 2.18E−24 | Chr4 | 101803507 |
| rs6837129 | rs7680366 | 0.892855 | 0.227203 | 9.12E−07 | Chr4 | 101804585 |
| rs11097727 | rs7680366 | 1 | 0.285005 | 1.79E−09 | Chr4 | 101806945 |
| rs4235457 | rs7680366 | 0.892855 | 0.227203 | 9.12E−07 | Chr4 | 101810497 |
| rs906600 | rs7680366 | 0.892855 | 0.227203 | 9.12E−07 | Chr4 | 101815546 |
| rs17633767 | rs7680366 | 0.769443 | 0.264906 | 3.09E−07 | Chr4 | 101815904 |
| rs906601 | rs7680366 | 1 | 0.222702 | 8.68E−10 | Chr4 | 101816201 |
| rs6848941 | rs7680366 | 1 | 0.231707 | 3.95E−10 | Chr4 | 101818877 |
| rs10516468 | rs7684457 | 1 | 0.201774 | 4.20E−09 | Chr4 | 101818931 |
| rs6823243 | rs7680366 | 1 | 0.263804 | 3.80E−11 | Chr4 | 101818977 |
| rs6823607 | rs7680366 | 0.892855 | 0.227203 | 9.12E−07 | Chr4 | 101819149 |
| rs11946360 | rs7684457 | 1 | 0.206852 | 4.71E−09 | Chr4 | 101819736 |
| rs11736383 | rs7680366 | 1 | 0.231707 | 3.95E−10 | Chr4 | 101819839 |
| rs10516467 | rs7680366 | 1 | 0.25879 | 5.18E−11 | Chr4 | 101820125 |
| rs6820972 | rs7680366 | 1 | 0.25879 | 5.18E−11 | Chr4 | 101821828 |
| rs7671042 | rs7680366 | 0.648625 | 0.246465 | 8.69E−07 | Chr4 | 101829767 |
| rs11097731 | rs7680366 | 0.924971 | 0.369246 | 1.92E−10 | Chr4 | 101834868 |
| rs722735 | rs7680366 | 1 | 0.286851 | 8.60E−09 | Chr4 | 101841069 |
| rs1396285 | rs7680366 | 0.884233 | 0.204986 | 3.71E−06 | Chr4 | 101844578 |
| rs7681329 | rs7680366 | 0.882366 | 0.354298 | 1.62E−10 | Chr4 | 101849314 |
| rs1396287 | rs7680366 | 0.874136 | 0.201981 | 7.85E−06 | Chr4 | 101853716 |
| rs9995730 | rs7680366 | 0.833066 | 0.371345 | 6.51E−10 | Chr4 | 101863227 |
| rs981272 | rs7680366 | 0.836454 | 0.346591 | 2.53E−10 | Chr4 | 101870136 |
| rs17572292 | rs7680366 | 0.83307 | 0.332514 | 6.04E−10 | Chr4 | 101870642 |
| rs10010359 | rs7680366 | 0.83307 | 0.332514 | 6.04E−10 | Chr4 | 101873020 |
| rs7687299 | rs7680366 | 0.836454 | 0.346591 | 2.53E−10 | Chr4 | 101880049 |
| rs1396282 | rs7680366 | 0.737969 | 0.464608 | 1.92E−12 | Chr4 | 101881360 |
| rs974858 | rs7680366 | 0.884233 | 0.204986 | 3.71E−06 | Chr4 | 101883863 |
| rs4145995 | rs7680366 | 0.640193 | 0.206451 | 0.000028 | Chr4 | 101936012 |
| rs7754000 | rs950286 | 0.897084 | 0.614908 | 1.56E−11 | Chr6 | 248017 |
| rs4959746 | rs1050975 | 1 | 0.230769 | 0.000136 | Chr6 | 289181 |
| rs3866815 | rs9378805 | 0.743467 | 0.40501 | 2.27E−11 | Chr6 | 324031 |
| rs2797297 | rs1050975 | 0.88031 | 0.247205 | 1.90E−06 | Chr6 | 325253 |
| rs2666954 | rs9328192 | 0.613937 | 0.37574 | 5.17E−11 | Chr6 | 325789 |
| rs2666955 | rs872071 | 0.688988 | 0.353391 | 8.63E−10 | Chr6 | 325920 |
| rs2797299 | rs872071 | 0.721817 | 0.395598 | 3.96E−11 | Chr6 | 326030 |
| rs2666956 | rs872071 | 0.713696 | 0.228049 | 6.63E−07 | Chr6 | 326329 |
| rs3914430 | rs9328192 | 0.682279 | 0.245911 | 3.54E−07 | Chr6 | 326918 |
| rs2797301 | rs872071 | 0.700821 | 0.402052 | 4.91E−12 | Chr6 | 327111 |
| rs4985288 | rs9328192 | 0.682279 | 0.245911 | 3.54E−07 | Chr6 | 327246 |
| rs9405192 | rs9328192 | 0.682279 | 0.245911 | 3.54E−07 | Chr6 | 327537 |
| rs1033180 | rs1050975 | 0.89899 | 0.734038 | 7.68E−13 | Chr6 | 328546 |
| rs1514346 | rs7757906 | 0.91249 | 0.283121 | 1.26E−08 | Chr6 | 334630 |
| rs6899334 | rs7757906 | 0.91249 | 0.283121 | 1.26E−08 | Chr6 | 335105 |
| rs6930635 | rs7757906 | 0.779456 | 0.248319 | 1.05E−07 | Chr6 | 340634 |
| rs1775589 | rs7757906 | 0.845614 | 0.275561 | 1.60E−08 | Chr6 | 342290 |
| rs6900384 | rs7757906 | 0.843806 | 0.274813 | 1.87E−08 | Chr6 | 342937 |
| rs2666970 | rs7757906 | 0.845614 | 0.275561 | 1.60E−08 | Chr6 | 343242 |
| rs2671422 | rs7757906 | 0.841954 | 0.274047 | 2.20E−08 | Chr6 | 343775 |
| rs1473037 | rs7757906 | 0.779456 | 0.248319 | 1.05E−07 | Chr6 | 344079 |
| rs2292383 | rs11242867 | 0.763158 | 0.340486 | 9.27E−09 | Chr6 | 347087 |
| rs13208928 | rs9503644 | 0.702786 | 0.309772 | 4.05E−08 | Chr6 | 348470 |
| rs1877179 | rs1050975 | 0.907407 | 0.823388 | 1.06E−14 | Chr6 | 348604 |
| rs3778607 | rs872071 | 0.863726 | 0.721523 | 1.54E−22 | Chr6 | 348799 |
| rs2001508 | rs9503644 | 1 | 0.863378 | 1.26E−23 | Chr6 | 349632 |
| rs17825664 | rs11242867 | 0.915789 | 0.455278 | 1.94E−11 | Chr6 | 350873 |
| rs1131442 | rs872071 | 1 | 0.52381 | 4.43E−19 | Chr6 | 352656 |
| rs1050975 | rs1050975 | 1 | 1 | 0 | Chr6 | 353012 |
| rs1050976 | rs872071 | 1 | 0.967105 | 9.59E−36 | Chr6 | 353079 |
| rs7768807 | rs872071 | 1 | 0.343186 | 3.92E−13 | Chr6 | 353246 |
| rs9391997 | rs872071 | 1 | 0.965928 | 4.29E−35 | Chr6 | 354119 |
| rs1877175 | rs7757906 | 0.897949 | 0.542394 | 1.45E−15 | Chr6 | 355493 |
| rs872071 | rs872071 | 1 | 1 | 0 | Chr6 | 356064 |
| rs6906608 | rs7757906 | 0.81318 | 0.210295 | 1.78E−06 | Chr6 | 356554 |
| rs11242865 | rs7757906 | 0.904157 | 0.572794 | 5.63E−17 | Chr6 | 356954 |
| rs11757491 | rs9503644 | 1 | 0.501887 | 5.12E−13 | Chr6 | 357236 |
| rs7757906 | rs7757906 | 1 | 1 | 0 | Chr6 | 357741 |
| rs4959853 | rs11242867 | 1 | 1 | 1.53E−28 | Chr6 | 358770 |
| rs9503644 | rs11242867 | 1 | 1 | 0 | Chr6 | 360406 |
| rs9378805 | rs9378805 | 1 | 1 | 0 | Chr6 | 362727 |
| rs9378374 | rs7757906 | 0.901319 | 0.570483 | 2.00E−16 | Chr6 | 367408 |
| rs7748534 | rs9328192 | 1 | 0.417722 | 2.38E−15 | Chr6 | 372152 |
| rs1473602 | rs9328192 | 1 | 0.204301 | 1.79E−08 | Chr6 | 373722 |
| rs950286 | rs950286 | 1 | 1 | 0 | Chr6 | 374457 |
| rs2048698 | rs9328192 | 1 | 0.966063 | 1.28E−34 | Chr6 | 378962 |
| rs7454545 | rs9328192 | 1 | 0.966063 | 1.28E−34 | Chr6 | 379348 |
| rs9328192 | rs9328192 | 1 | 1 | 0 | Chr6 | 379364 |
| rs13210344 | rs9405681 | 0.792475 | 0.281616 | 3.32E−06 | Chr6 | 386245 |
| rs6920655 | rs9405681 | 1 | 0.637363 | 2.13E−20 | Chr6 | 386883 |
| rs4959880 | rs950286 | 0.908887 | 0.414596 | 3.50E−10 | Chr6 | 387206 |
| rs7749710 | rs9405681 | 0.892508 | 0.288159 | 6.91E−08 | Chr6 | 387410 |
| rs9405675 | rs9405675 | 1 | 1 | 0 | Chr6 | 389600 |
| rs10900949 | rs950286 | 0.913524 | 0.833548 | 1.02E−14 | Chr6 | 390278 |
| rs7767018 | rs9405681 | 1 | 0.448276 | 1.77E−12 | Chr6 | 393453 |
| rs9392537 | rs9405675 | 1 | 0.38792 | 1.27E−11 | Chr6 | 393953 |
| rs9405681 | rs9405681 | 1 | 1 | 0 | Chr6 | 394358 |
| rs13214605 | rs9405681 | 1 | 0.911012 | 1.76E−26 | Chr6 | 394483 |
| rs12180765 | rs9405681 | 1 | 1 | 9.74E−28 | Chr6 | 394737 |
| rs6899601 | rs9405681 | 1 | 0.263006 | 2.62E−07 | Chr6 | 396099 |
| rs4339511 | rs4959270 | 1 | 0.721924 | 1.06E−24 | Chr6 | 396895 |
| rs4311550 | rs4959270 | 1 | 0.838319 | 4.84E−29 | Chr6 | 396925 |
| rs908026 | rs4959270 | 1 | 0.870724 | 3.27E−30 | Chr6 | 400419 |
| rs4959270 | rs4959270 | 1 | 1 | 0 | Chr6 | 402748 |
| rs11242899 | rs1540771 | 0.931733 | 0.340991 | 2.53E−10 | Chr6 | 405302 |
| rs2316795 | rs9405681 | 0.898563 | 0.700787 | 4.70E−19 | Chr6 | 405732 |
| rs1113387 | rs4959270 | 0.964028 | 0.867481 | 4.00E−28 | Chr6 | 405901 |
| rs962517 | rs1540771 | 0.897599 | 0.805684 | 1.45E−25 | Chr6 | 407299 |
| rs12661290 | rs1540771 | 0.934467 | 0.359565 | 6.77E−11 | Chr6 | 407341 |
| rs908025 | rs9405681 | 1 | 0.665622 | 1.60E−18 | Chr6 | 408233 |
| rs11961808 | rs1540771 | 0.813694 | 0.299835 | 7.16E−09 | Chr6 | 408479 |
| rs908024 | rs4959270 | 0.869235 | 0.296844 | 7.12E−09 | Chr6 | 408753 |
| rs908023 | rs9405681 | 1 | 0.735489 | 4.92E−20 | Chr6 | 409093 |
| rs868094 | rs1540771 | 0.897532 | 0.804352 | 2.43E−25 | Chr6 | 409426 |
| rs7454852 | rs1540771 | 0.868742 | 0.308587 | 3.31E−09 | Chr6 | 410501 |
| rs1540771 | rs1540771 | 1 | 1 | 0 | Chr6 | 411033 |
| rs1540767 | rs1540771 | 0.734392 | 0.288479 | 2.74E−08 | Chr6 | 411368 |
| rs4959273 | rs950039 | 0.793739 | 0.205837 | 4.15E−06 | Chr6 | 420810 |
| rs11242909 | rs950039 | 0.526827 | 0.268377 | 8.25E−08 | Chr6 | 422065 |
| rs7750350 | rs950039 | 0.526827 | 0.268377 | 8.25E−08 | Chr6 | 422738 |
| rs7750535 | rs950039 | 0.526827 | 0.268377 | 8.25E−08 | Chr6 | 422873 |
| rs11242912 | rs950039 | 0.809988 | 0.375509 | 8.09E−10 | Chr6 | 424926 |
| rs9504016 | rs950039 | 0.778046 | 0.289476 | 7.10E−08 | Chr6 | 425107 |
| rs9392573 | rs950039 | 0.805031 | 0.368228 | 1.98E−09 | Chr6 | 425628 |
| rs9392574 | rs950039 | 0.720234 | 0.391639 | 8.10E−11 | Chr6 | 425753 |
| rs10900954 | rs950039 | 0.95285 | 0.7692 | 3.56E−21 | Chr6 | 428817 |
| rs974455 | rs950039 | 0.943353 | 0.600276 | 4.41E−16 | Chr6 | 430078 |
| rs11242914 | rs950039 | 0.797229 | 0.495222 | 5.20E−13 | Chr6 | 430586 |
| rs12952 | rs950039 | 1 | 0.806161 | 2.74E−24 | Chr6 | 431193 |
| rs4072107 | rs950039 | 1 | 0.804772 | 9.10E−24 | Chr6 | 431953 |
| rs950039 | rs950039 | 1 | 1 | 0 | Chr6 | 438976 |
| rs4959951 | rs950039 | 1 | 0.478135 | 2.60E−14 | Chr6 | 439567 |
| rs9405242 | rs950039 | 0.881275 | 0.379944 | 4.12E−11 | Chr6 | 441555 |
| rs1473909 | rs950039 | 0.878045 | 0.365123 | 5.26E−11 | Chr6 | 445956 |
| rs12206548 | rs1540771 | 0.495635 | 0.237438 | 1.31E−06 | Chr6 | 451231 |
| rs13192740 | rs1540771 | 0.50554 | 0.25557 | 2.31E−07 | Chr6 | 459991 |
| rs9392618 | rs1540771 | 0.499935 | 0.24811 | 5.91E−07 | Chr6 | 460393 |
| rs9392056 | rs1540771 | 0.499869 | 0.217654 | 3.30E−06 | Chr6 | 463078 |
| rs2050134 | rs1540771 | 0.890774 | 0.202389 | 9.92E−06 | Chr6 | 472815 |
| rs6903640 | rs1540771 | 0.529325 | 0.2726 | 1.18E−07 | Chr6 | 473568 |
| rs2493034 | rs9328192 | 0.647708 | 0.217276 | 1.93E−06 | Chr6 | 473736 |
| rs4960043 | rs1540771 | 0.531291 | 0.272782 | 1.58E−07 | Chr6 | 479527 |
| rs6923301 | rs1540771 | 0.500849 | 0.211897 | 7.50E−06 | Chr6 | 485016 |
| rs6918152 | rs1540771 | 0.53818 | 0.276109 | 1.26E−07 | Chr6 | 487159 |
| rs3799296 | rs1540771 | 0.498458 | 0.213238 | 8.46E−06 | Chr6 | 487416 |
| rs2982494 | rs950286 | 0.767535 | 0.212027 | 0.000221 | Chr6 | 521032 |
| rs4960175 | rs950039 | 0.536932 | 0.252654 | 7.50E−07 | Chr6 | 615871 |
| rs2317079 | rs950039 | 0.493077 | 0.213035 | 4.42E−06 | Chr6 | 634565 |
| rs1251283 | rs10809808 | 0.650592 | 0.232911 | 1.98E−07 | Chr9 | 12500835 |
| rs791668 | rs10809808 | 0.554461 | 0.295891 | 4.63E−08 | Chr9 | 12505545 |
| rs791672 | rs10809808 | 0.768082 | 0.37145 | 2.39E−11 | Chr9 | 12507596 |
| rs791675 | rs10809808 | 0.768082 | 0.37145 | 2.39E−11 | Chr9 | 12509087 |
| rs791681 | rs10809808 | 0.768082 | 0.37145 | 2.39E−11 | Chr9 | 12512037 |
| rs1325131 | rs10809808 | 0.764208 | 0.355469 | 6.35E−11 | Chr9 | 12512752 |
| rs10756375 | rs10809808 | 0.764208 | 0.355469 | 6.35E−11 | Chr9 | 12513291 |
| rs1590487 | rs1408799 | 0.6 | 0.221053 | 6.62E−07 | Chr9 | 12514085 |
| rs702131 | rs10809808 | 0.496655 | 0.20744 | 0.000014 | Chr9 | 12516192 |
| rs791688 | rs10809808 | 0.605481 | 0.221056 | 2.13E−06 | Chr9 | 12517414 |
| rs791691 | rs10809808 | 0.771832 | 0.388065 | 8.64E−12 | Chr9 | 12517911 |
| rs791694 | rs10809808 | 0.832206 | 0.253836 | 1.84E−07 | Chr9 | 12519788 |
| rs791696 | rs10809808 | 0.774742 | 0.401814 | 4.15E−12 | Chr9 | 12520255 |
| rs791697 | rs10809808 | 0.771832 | 0.388065 | 8.64E−12 | Chr9 | 12520324 |
| rs1251295 | rs10809808 | 0.498808 | 0.214521 | 2.59E−06 | Chr9 | 12520725 |
| rs702132 | rs10809808 | 0.768082 | 0.37145 | 2.39E−11 | Chr9 | 12522047 |
| rs702133 | rs10809808 | 0.750973 | 0.368438 | 1.70E−10 | Chr9 | 12522274 |
| rs702134 | rs10809808 | 0.768082 | 0.37145 | 2.39E−11 | Chr9 | 12522458 |
| rs791699 | rs10809808 | 0.768082 | 0.37145 | 2.39E−11 | Chr9 | 12522550 |
| rs7046025 | rs10809808 | 0.810626 | 0.215527 | 4.12E−06 | Chr9 | 12524779 |
| rs1570781 | rs10809808 | 0.59442 | 0.304641 | 6.29E−09 | Chr9 | 12527279 |
| rs10809792 | rs10809808 | 0.59442 | 0.304641 | 6.29E−09 | Chr9 | 12529953 |
| rs10960702 | rs1022901 | 1 | 0.275945 | 3.70E−07 | Chr9 | 12530169 |
| rs12000969 | rs1022901 | 1 | 0.318182 | 4.45E−08 | Chr9 | 12531971 |
| rs1408808 | rs10809808 | 0.59442 | 0.304641 | 6.29E−09 | Chr9 | 12532187 |
| rs2025555 | rs10809808 | 0.588532 | 0.288059 | 1.66E−08 | Chr9 | 12536631 |
| rs1408809 | rs10809808 | 0.59442 | 0.304641 | 6.29E−09 | Chr9 | 12537685 |
| rs10124086 | rs10809808 | 0.682369 | 0.241768 | 6.89E−07 | Chr9 | 12539675 |
| rs7866061 | rs1022901 | 1 | 0.275945 | 3.70E−07 | Chr9 | 12540853 |
| rs10122091 | rs10809808 | 0.660116 | 0.240244 | 5.07E−06 | Chr9 | 12542369 |
| rs2104400 | rs10809808 | 0.667478 | 0.219407 | 2.64E−06 | Chr9 | 12557981 |
| rs10809794 | rs10809808 | 0.682369 | 0.241768 | 6.89E−07 | Chr9 | 12564371 |
| rs1125108 | rs10809808 | 0.682369 | 0.241768 | 6.89E−07 | Chr9 | 12564538 |
| rs16923032 | rs1022901 | 1 | 0.275945 | 3.70E−07 | Chr9 | 12564724 |
| rs10809795 | rs10809808 | 0.668034 | 0.241916 | 1.91E−06 | Chr9 | 12566604 |
| rs10960708 | rs10809808 | 0.861864 | 0.483878 | 4.97E−14 | Chr9 | 12568438 |
| rs10809797 | rs1022901 | 1 | 0.541206 | 4.18E−13 | Chr9 | 12571270 |
| rs1325154 | rs1022901 | 0.897959 | 0.399093 | 1.64E−09 | Chr9 | 12572565 |
| rs10429629 | rs10809808 | 0.806767 | 0.626767 | 5.71E−18 | Chr9 | 12572787 |
| rs10960710 | rs10809808 | 0.806767 | 0.626767 | 5.71E−18 | Chr9 | 12577153 |
| rs1022901 | rs1022901 | 1 | 1 | 0 | Chr9 | 12578259 |
| rs962298 | rs10809808 | 0.689196 | 0.39002 | 1.88E−10 | Chr9 | 12578950 |
| rs6474717 | rs10809808 | 0.804147 | 0.59992 | 3.40E−17 | Chr9 | 12579068 |
| rs10809800 | rs10809808 | 0.699666 | 0.267558 | 8.89E−08 | Chr9 | 12579981 |
| rs1325112 | rs1022901 | 0.825073 | 0.400507 | 1.44E−09 | Chr9 | 12582912 |
| rs1325113 | rs1022901 | 1 | 0.404959 | 5.41E−10 | Chr9 | 12583080 |
| rs4428755 | rs1022901 | 0.941425 | 0.832852 | 6.60E−20 | Chr9 | 12583124 |
| rs1359442 | rs10809808 | 1 | 0.296703 | 5.60E−10 | Chr9 | 12584030 |
| rs10756379 | rs10809808 | 1 | 0.252336 | 1.09E−08 | Chr9 | 12584850 |
| rs10756380 | rs1022901 | 1 | 0.404959 | 5.41E−10 | Chr9 | 12584967 |
| rs1157330 | rs10809808 | 1 | 0.3361 | 1.50E−10 | Chr9 | 12585352 |
| rs10756384 | rs1022901 | 0.888683 | 0.355028 | 1.50E−08 | Chr9 | 12586589 |
| rs13283146 | rs10809808 | 0.95548 | 0.745158 | 3.86E−21 | Chr9 | 12589561 |
| rs10809802 | rs10809808 | 1 | 0.252336 | 1.09E−08 | Chr9 | 12589803 |
| rs4741238 | rs10809808 | 1 | 0.252336 | 1.09E−08 | Chr9 | 12591152 |
| rs1408790 | rs10809808 | 1 | 0.962477 | 2.03E−32 | Chr9 | 12592681 |
| rs1408791 | rs1022901 | 1 | 0.494949 | 5.52E−12 | Chr9 | 12592864 |
| rs1325114 | rs10809808 | 1 | 0.22899 | 2.27E−07 | Chr9 | 12593853 |
| rs10960716 | rs10809808 | 0.961014 | 0.888892 | 9.82E−28 | Chr9 | 12594407 |
| rs713596 | rs10809808 | 1 | 1 | 8.83E−35 | Chr9 | 12595687 |
| rs1925236 | rs10809808 | 1 | 0.252336 | 1.09E−08 | Chr9 | 12597437 |
| rs10738285 | rs10809808 | 1 | 0.252336 | 1.38E−08 | Chr9 | 12597670 |
| rs1325115 | rs1022901 | 0.905808 | 0.444054 | 1.65E−10 | Chr9 | 12598182 |
| rs1325116 | rs1022901 | 0.886578 | 0.354086 | 1.79E−08 | Chr9 | 12598432 |
| rs1408792 | rs1022901 | 1 | 0.426087 | 4.93E−10 | Chr9 | 12599014 |
| rs10809806 | rs10809808 | 1 | 0.661877 | 5.58E−21 | Chr9 | 12601123 |
| rs13288558 | rs10809808 | 1 | 1 | 8.83E−35 | Chr9 | 12602529 |
| rs1359443 | rs10809808 | 1 | 0.252336 | 1.09E−08 | Chr9 | 12602627 |
| rs1359444 | rs10809808 | 1 | 0.390708 | 1.01E−12 | Chr9 | 12602897 |
| rs2025556 | rs1022901 | 1 | 0.404959 | 5.41E−10 | Chr9 | 12603216 |
| rs1325117 | rs10809808 | 1 | 1 | 5.47E−30 | Chr9 | 12603472 |
| rs6474718 | rs10809808 | 1 | 0.661877 | 5.58E−21 | Chr9 | 12604387 |
| rs6474719 | rs10809808 | 1 | 0.390708 | 1.01E−12 | Chr9 | 12604610 |
| rs13283649 | rs10809808 | 1 | 0.962049 | 2.16E−32 | Chr9 | 12608337 |
| rs1575692 | rs10809808 | 1 | 0.319527 | 1.22E−10 | Chr9 | 12609065 |
| rs1325118 | rs10809808 | 0.957671 | 0.784336 | 1.47E−23 | Chr9 | 12609616 |
| rs10738286 | rs1022901 | 0.887661 | 0.350671 | 4.65E−08 | Chr9 | 12609795 |
| rs7466934 | rs10809808 | 1 | 0.962963 | 9.79E−33 | Chr9 | 12609840 |
| rs10960721 | rs10809808 | 1 | 0.519231 | 1.53E−16 | Chr9 | 12610116 |
| rs7036899 | rs10809808 | 1 | 0.962963 | 9.79E−33 | Chr9 | 12610266 |
| rs10756386 | rs10809808 | 1 | 0.962963 | 9.79E−33 | Chr9 | 12611004 |
| rs1325120 | rs10809808 | 1 | 0.319527 | 1.22E−10 | Chr9 | 12612642 |
| rs10960723 | rs10809808 | 1 | 1 | 6.04E−34 | Chr9 | 12612878 |
| rs4612469 | rs1022901 | 1 | 0.404959 | 5.41E−10 | Chr9 | 12612925 |
| rs977888 | rs10809808 | 1 | 0.962963 | 9.79E−33 | Chr9 | 12614357 |
| rs10809808 | rs10809808 | 1 | 1 | 0 | Chr9 | 12614463 |
| rs2181818 | rs10809808 | 1 | 0.252336 | 1.09E−08 | Chr9 | 12614629 |
| rs10738287 | rs1022901 | 1 | 0.379845 | 4.01E−09 | Chr9 | 12616313 |
| rs981945 | rs10809808 | 1 | 0.296703 | 5.60E−10 | Chr9 | 12616966 |
| rs1408793 | rs10809808 | 1 | 0.319527 | 1.22E−10 | Chr9 | 12618213 |
| rs10756387 | rs1022901 | 1 | 0.404959 | 5.41E−10 | Chr9 | 12618599 |
| rs10960730 | rs10809808 | 1 | 1 | 8.83E−35 | Chr9 | 12621099 |
| rs10809809 | rs10809808 | 1 | 1 | 1.31E−34 | Chr9 | 12621398 |
| rs10125059 | rs1022901 | 1 | 0.404959 | 5.41E−10 | Chr9 | 12621525 |
| rs10756388 | rs1408799 | 1 | 0.358974 | 2.20E−11 | Chr9 | 12622930 |
| rs10960731 | rs1022901 | 0.888683 | 0.355028 | 1.50E−08 | Chr9 | 12623322 |
| rs10960732 | rs10809808 | 1 | 1 | 8.83E−35 | Chr9 | 12623495 |
| rs7026116 | rs10809808 | 1 | 1 | 2.90E−34 | Chr9 | 12623981 |
| rs10756390 | rs10809808 | 1 | 0.252336 | 1.09E−08 | Chr9 | 12625712 |
| rs10124166 | rs1022901 | 1 | 0.404959 | 5.41E−10 | Chr9 | 12627846 |
| rs10960734 | rs10809808 | 1 | 0.319527 | 1.22E−10 | Chr9 | 12628235 |
| rs7047297 | rs10809808 | 1 | 0.927733 | 2.76E−31 | Chr9 | 12628540 |
| rs13301970 | rs10809808 | 0.952924 | 0.679664 | 8.69E−19 | Chr9 | 12629877 |
| rs10960735 | rs10809808 | 1 | 0.925094 | 2.31E−29 | Chr9 | 12631821 |
| rs1325122 | rs10809808 | 1 | 0.962963 | 9.79E−33 | Chr9 | 12632878 |
| rs6474720 | rs1022901 | 0.888683 | 0.355028 | 1.50E−08 | Chr9 | 12633558 |
| rs6474721 | rs1022901 | 0.887661 | 0.350671 | 4.65E−08 | Chr9 | 12633660 |
| rs4740525 | rs10809808 | 1 | 0.296703 | 5.60E−10 | Chr9 | 12634782 |
| rs1155509 | rs10809808 | 1 | 0.319527 | 1.22E−10 | Chr9 | 12637332 |
| rs10960738 | rs10809808 | 0.905849 | 0.642125 | 1.27E−16 | Chr9 | 12638831 |
| rs13283345 | rs10809808 | 0.905842 | 0.636141 | 4.58E−17 | Chr9 | 12640198 |
| rs9657586 | rs10809808 | 1 | 0.440559 | 3.45E−14 | Chr9 | 12640288 |
| rs10809811 | rs10809808 | 1 | 0.927733 | 2.76E−31 | Chr9 | 12640996 |
| rs1408794 | rs10809808 | 1 | 0.927733 | 2.76E−31 | Chr9 | 12641340 |
| rs1408795 | rs10809808 | 0.908546 | 0.650976 | 3.99E−18 | Chr9 | 12641413 |
| rs13294940 | rs1408799 | 1 | 0.636364 | 2.93E−19 | Chr9 | 12642364 |
| rs1325124 | rs1022901 | 0.795918 | 0.313544 | 1.11E−07 | Chr9 | 12642651 |
| rs996697 | rs1408799 | 1 | 0.466667 | 1.86E−14 | Chr9 | 12642983 |
| rs996696 | rs10809808 | 1 | 0.252336 | 1.09E−08 | Chr9 | 12643270 |
| rs2382358 | rs10809808 | 1 | 0.252336 | 1.09E−08 | Chr9 | 12643796 |
| rs2382359 | rs1408799 | 1 | 0.397993 | 2.82E−12 | Chr9 | 12643846 |
| rs995263 | rs10809808 | 1 | 0.962963 | 9.79E−33 | Chr9 | 12644578 |
| rs1325125 | rs1408799 | 1 | 0.340278 | 2.26E−10 | Chr9 | 12645862 |
| rs10435754 | rs10809808 | 1 | 0.5086 | 4.82E−16 | Chr9 | 12647603 |
| rs4741242 | rs1022901 | 0.793388 | 0.311553 | 2.21E−07 | Chr9 | 12649691 |
| rs2209275 | rs1408799 | 1 | 0.553265 | 5.41E−17 | Chr9 | 12653234 |
| rs10123110 | rs10809808 | 1 | 0.366516 | 6.12E−12 | Chr9 | 12656092 |
| rs7022317 | rs10809808 | 0.94829 | 0.595534 | 3.85E−17 | Chr9 | 12656686 |
| rs1121541 | rs10809808 | 1 | 0.927733 | 2.76E−31 | Chr9 | 12657049 |
| rs10809818 | rs10809808 | 0.875959 | 0.682985 | 1.40E−19 | Chr9 | 12658121 |
| rs1325127 | rs10809808 | 0.875959 | 0.682985 | 1.40E−19 | Chr9 | 12658328 |
| rs10960748 | rs10809808 | 1 | 0.927733 | 2.76E−31 | Chr9 | 12658805 |
| rs9298679 | rs1408799 | 1 | 0.677419 | 8.55E−21 | Chr9 | 12659346 |
| rs9298680 | rs1408799 | 1 | 0.283489 | 2.93E−09 | Chr9 | 12659377 |
| rs7863161 | rs1408799 | 1 | 0.283489 | 2.93E−09 | Chr9 | 12659735 |
| rs1041105 | rs1408799 | 1 | 0.283489 | 2.93E−09 | Chr9 | 12661059 |
| rs10960749 | rs10809808 | 1 | 0.89418 | 4.71E−30 | Chr9 | 12661566 |
| rs1408799 | rs1408799 | 1 | 1 | 0 | Chr9 | 12662097 |
| rs1408800 | rs1408799 | 1 | 1 | 9.56E−34 | Chr9 | 12662275 |
| rs13294134 | rs10809808 | 1 | 0.89418 | 4.71E−30 | Chr9 | 12663636 |
| rs16929340 | rs10809808 | 0.810468 | 0.341061 | 2.99E−09 | Chr9 | 12664124 |
| rs13299830 | rs10809808 | 0.835386 | 0.43355 | 1.99E−11 | Chr9 | 12664531 |
| rs10960751 | rs10809808 | 1 | 0.9273 | 6.09E−31 | Chr9 | 12665264 |
| rs10960752 | rs10809808 | 1 | 0.9273 | 6.09E−31 | Chr9 | 12665284 |
| rs10960753 | rs10809808 | 1 | 0.962512 | 4.40E−32 | Chr9 | 12665522 |
| rs16929342 | rs1408799 | 1 | 0.212121 | 2.92E−07 | Chr9 | 12665661 |
| rs16929345 | rs1022901 | 0.777365 | 0.271656 | 8.52E−07 | Chr9 | 12666236 |
| rs16929346 | rs1408799 | 1 | 0.308176 | 5.94E−10 | Chr9 | 12666417 |
| rs13296454 | rs10809808 | 1 | 0.89418 | 4.71E−30 | Chr9 | 12667181 |
| rs13297008 | rs10809808 | 1 | 0.89418 | 4.71E−30 | Chr9 | 12667471 |
| rs10116013 | rs10809808 | 0.940181 | 0.498947 | 8.84E−14 | Chr9 | 12667979 |
| rs10809826 | rs10809808 | 1 | 0.927733 | 2.76E−31 | Chr9 | 12672663 |
| rs7847593 | rs1408799 | 1 | 0.212121 | 2.92E−07 | Chr9 | 12673639 |
| rs13293905 | rs10809808 | 0.909909 | 0.652245 | 1.47E−18 | Chr9 | 12675943 |
| rs11791497 | rs1022901 | 1 | 0.318182 | 4.45E−08 | Chr9 | 12677872 |
| rs11787999 | rs1022901 | 1 | 0.318182 | 4.45E−08 | Chr9 | 12683732 |
| rs2762460 | rs10809808 | 1 | 0.889094 | 6.61E−29 | Chr9 | 12686478 |
| rs2762461 | rs10809808 | 1 | 0.862188 | 5.85E−29 | Chr9 | 12686499 |
| rs2762462 | rs927869 | 1 | 0.586426 | 5.71E−19 | Chr9 | 12689776 |
| rs2762463 | rs10809808 | 0.86839 | 0.619199 | 3.71E−17 | Chr9 | 12691897 |
| rs2224863 | rs927869 | 0.829922 | 0.568776 | 3.86E−16 | Chr9 | 12692890 |
| rs2733830 | rs10809808 | 0.86718 | 0.614984 | 8.03E−17 | Chr9 | 12693359 |
| rs2733831 | rs10809808 | 1 | 0.89418 | 4.71E−30 | Chr9 | 12693484 |
| rs17280279 | rs1022901 | 1 | 0.318182 | 4.45E−08 | Chr9 | 12693991 |
| rs2733832 | rs10809808 | 1 | 0.83165 | 5.77E−28 | Chr9 | 12694725 |
| rs2733833 | rs10809808 | 0.829508 | 0.588452 | 3.41E−16 | Chr9 | 12695095 |
| rs2209277 | rs10809808 | 0.86839 | 0.619199 | 3.71E−17 | Chr9 | 12696236 |
| rs2209278 | rs1022901 | 1 | 0.318182 | 4.45E−08 | Chr9 | 12696652 |
| rs10809828 | rs10809808 | 0.862583 | 0.368966 | 1.74E−09 | Chr9 | 12697861 |
| rs2733834 | rs927869 | 0.86733 | 0.596844 | 3.30E−16 | Chr9 | 12698910 |
| rs683 | rs927869 | 0.834698 | 0.598446 | 4.29E−17 | Chr9 | 12699305 |
| rs2762464 | rs927869 | 0.872594 | 0.628769 | 4.43E−18 | Chr9 | 12699586 |
| rs910 | rs927869 | 0.955183 | 0.69502 | 7.59E−21 | Chr9 | 12700035 |
| rs1063380 | rs927869 | 0.955183 | 0.69502 | 7.59E−21 | Chr9 | 12700090 |
| rs9298681 | rs927869 | 1 | 0.409326 | 2.74E−13 | Chr9 | 12701032 |
| rs768617 | rs1022901 | 1 | 0.318182 | 4.45E−08 | Chr9 | 12705816 |
| rs3891858 | rs1022901 | 1 | 0.318182 | 4.45E−08 | Chr9 | 12706172 |
| rs10960758 | rs927869 | 1 | 0.960409 | 1.40E−31 | Chr9 | 12706315 |
| rs10960759 | rs927869 | 1 | 0.963834 | 2.55E−33 | Chr9 | 12706428 |
| rs12379024 | rs927869 | 1 | 0.963834 | 2.55E−33 | Chr9 | 12707405 |
| rs13295868 | rs927869 | 1 | 0.963834 | 2.55E−33 | Chr9 | 12707912 |
| rs7019226 | rs927869 | 1 | 0.929349 | 7.53E−32 | Chr9 | 12708370 |
| rs11789751 | rs927869 | 1 | 0.962441 | 7.45E−32 | Chr9 | 12709264 |
| rs10491744 | rs927869 | 1 | 0.963834 | 2.55E−33 | Chr9 | 12710106 |
| rs10960760 | rs927869 | 1 | 0.963834 | 2.55E−33 | Chr9 | 12710152 |
| rs2382361 | rs927869 | 1 | 0.963834 | 2.55E−33 | Chr9 | 12710786 |
| rs1409626 | rs927869 | 1 | 0.963834 | 2.55E−33 | Chr9 | 12710820 |
| rs1409630 | rs927869 | 1 | 0.929349 | 7.53E−32 | Chr9 | 12711251 |
| rs7040346 | rs1022901 | 1 | 0.318182 | 4.45E−08 | Chr9 | 12711691 |
| rs13288475 | rs927869 | 1 | 0.929349 | 7.53E−32 | Chr9 | 12711714 |
| rs13288636 | rs927869 | 1 | 0.929349 | 7.53E−32 | Chr9 | 12711806 |
| rs13288681 | rs927869 | 1 | 0.927602 | 1.75E−31 | Chr9 | 12711881 |
| rs1326798 | rs927869 | 1 | 0.929349 | 7.53E−32 | Chr9 | 12712227 |
| rs7871257 | rs927869 | 0.879475 | 0.412756 | 1.74E−11 | Chr9 | 12712357 |
| rs12379260 | rs927869 | 1 | 0.929349 | 7.53E−32 | Chr9 | 12713112 |
| rs16929400 | rs1022901 | 1 | 0.318182 | 4.45E−08 | Chr9 | 12713131 |
| rs13284453 | rs927869 | 1 | 0.855497 | 5.72E−28 | Chr9 | 12714280 |
| rs13284898 | rs927869 | 1 | 0.925766 | 4.07E−31 | Chr9 | 12714560 |
| rs12001299 | rs1022901 | 1 | 0.318182 | 4.45E−08 | Chr9 | 12718887 |
| rs7025758 | rs1022901 | 1 | 0.318182 | 4.45E−08 | Chr9 | 12720636 |
| rs7048117 | rs927869 | 1 | 0.481707 | 9.85E−16 | Chr9 | 12725950 |
| rs10756400 | rs927869 | 0.951251 | 0.662474 | 1.69E−16 | Chr9 | 12728157 |
| rs970944 | rs927869 | 0.952135 | 0.635196 | 7.02E−19 | Chr9 | 12728401 |
| rs970945 | rs927869 | 0.952135 | 0.635196 | 7.02E−19 | Chr9 | 12728641 |
| rs970946 | rs927869 | 0.952135 | 0.635196 | 7.02E−19 | Chr9 | 12728690 |
| rs970947 | rs927869 | 0.952135 | 0.635196 | 7.02E−19 | Chr9 | 12728813 |
| rs10960774 | rs927869 | 1 | 0.963834 | 2.55E−33 | Chr9 | 12729313 |
| rs10756402 | rs927869 | 0.948197 | 0.628457 | 2.21E−16 | Chr9 | 12729948 |
| rs10756403 | rs927869 | 0.928208 | 0.458398 | 5.62E−11 | Chr9 | 12730760 |
| rs10738290 | rs927869 | 0.937539 | 0.445695 | 9.41E−13 | Chr9 | 12730906 |
| rs13300005 | rs1408799 | 0.902955 | 0.271776 | 4.08E−08 | Chr9 | 12738191 |
| rs10756406 | rs927869 | 1 | 1 | 2.19E−35 | Chr9 | 12738587 |
| rs7019486 | rs927869 | 1 | 0.496855 | 7.89E−16 | Chr9 | 12738633 |
| rs927868 | rs927869 | 0.961918 | 0.887348 | 3.29E−27 | Chr9 | 12738795 |
| rs7019981 | rs927869 | 1 | 0.481707 | 9.85E−16 | Chr9 | 12738818 |
| rs927869 | rs927869 | 1 | 1 | 0 | Chr9 | 12738962 |
| rs4741245 | rs927869 | 1 | 1 | 2.19E−35 | Chr9 | 12739300 |
| rs7023927 | rs927869 | 1 | 1 | 2.19E−35 | Chr9 | 12739596 |
| rs7035500 | rs927869 | 1 | 1 | 5.08E−35 | Chr9 | 12740095 |
| rs13302551 | rs927869 | 1 | 0.963834 | 2.55E−33 | Chr9 | 12740812 |
| rs1543587 | rs927869 | 1 | 1 | 2.19E−35 | Chr9 | 12741741 |
| rs1074789 | rs927869 | 1 | 0.963415 | 4.90E−33 | Chr9 | 12742340 |
| rs2181816 | rs927869 | 1 | 0.481707 | 9.85E−16 | Chr9 | 12742760 |
| rs10125771 | rs927869 | 1 | 0.385366 | 7.84E−13 | Chr9 | 12747058 |
| rs10960779 | rs927869 | 1 | 0.963415 | 4.90E−33 | Chr9 | 12748881 |
| rs1326789 | rs927869 | 0.962428 | 0.924948 | 1.65E−28 | Chr9 | 12749838 |
| rs7025842 | rs927869 | 0.962952 | 0.927276 | 2.23E−30 | Chr9 | 12750647 |
| rs7025953 | rs927869 | 0.962952 | 0.927276 | 2.23E−30 | Chr9 | 12750718 |
| rs7025771 | rs927869 | 0.962952 | 0.927276 | 2.23E−30 | Chr9 | 12750762 |
| rs7025914 | rs927869 | 0.962476 | 0.892858 | 6.36E−29 | Chr9 | 12750884 |
| rs10491743 | rs927869 | 0.962952 | 0.927276 | 2.23E−30 | Chr9 | 12750920 |
| rs1326790 | rs927869 | 0.962952 | 0.927276 | 2.23E−30 | Chr9 | 12751168 |
| rs1326791 | rs927869 | 0.96214 | 0.923172 | 7.10E−28 | Chr9 | 12751300 |
| rs1326792 | rs927869 | 0.962952 | 0.927276 | 2.23E−30 | Chr9 | 12751360 |
| rs7030485 | rs927869 | 0.96126 | 0.922928 | 1.47E−27 | Chr9 | 12751819 |
| rs10960781 | rs927869 | 0.960565 | 0.856422 | 5.77E−27 | Chr9 | 12752374 |
| rs12115198 | rs927869 | 1 | 0.892921 | 1.36E−29 | Chr9 | 12753450 |
| rs10960783 | rs927869 | 0.957714 | 0.848612 | 9.35E−24 | Chr9 | 12753809 |
| rs1041176 | rs927869 | 1 | 0.456877 | 5.58E−15 | Chr9 | 12754311 |
| rs10119113 | rs927869 | 0.93606 | 0.422076 | 1.26E−12 | Chr9 | 12755117 |
| rs16929473 | rs1022901 | 1 | 0.275945 | 3.94E−07 | Chr9 | 12757086 |
| rs1326795 | rs1408799 | 0.902955 | 0.271776 | 4.08E−08 | Chr9 | 12760108 |
| rs11793280 | rs1022901 | 1 | 0.275945 | 3.70E−07 | Chr9 | 12761667 |
| rs2209273 | rs927869 | 1 | 0.31802 | 7.92E−11 | Chr9 | 12762498 |
| rs7855624 | rs1408799 | 0.950398 | 0.671652 | 2.29E−19 | Chr9 | 12763263 |
| rs10491742 | rs927869 | 0.920051 | 0.755852 | 4.17E−23 | Chr9 | 12765488 |
| rs3750502 | rs1408799 | 0.928555 | 0.402367 | 1.12E−11 | Chr9 | 12766516 |
| rs4930643 | rs3750965 | 1 | 1 | 1.38E−30 | Chr11 | 68575512 |
| rs4930644 | rs896978 | 1 | 1 | 1.12E−27 | Chr11 | 68575673 |
| rs7940235 | rs2305498 | 0.940997 | 0.792145 | 9.37E−19 | Chr11 | 68576697 |
| rs11604251 | rs896978 | 1 | 1 | 2.32E−28 | Chr11 | 68577005 |
| rs10896398 | rs3750965 | 1 | 0.409499 | 8.52E−15 | Chr11 | 68577192 |
| rs12285715 | rs3750965 | 1 | 1 | 1.04E−30 | Chr11 | 68578224 |
| rs12285865 | rs3750965 | 1 | 0.944611 | 3.74E−24 | Chr11 | 68578438 |
| rs7127082 | rs2305498 | 0.943207 | 0.842765 | 3.54E−20 | Chr11 | 68578599 |
| rs3019776 | rs896978 | 0.784645 | 0.235626 | 3.14E−06 | Chr11 | 68582731 |
| rs896978 | rs896978 | 1 | 1 | 0 | Chr11 | 68585505 |
| rs11228469 | rs3750965 | 1 | 0.463782 | 2.74E−15 | Chr11 | 68586214 |
| rs3750972 | rs3750965 | 1 | 0.442601 | 1.16E−15 | Chr11 | 68587204 |
| rs10750839 | rs3750965 | 1 | 0.416737 | 4.84E−15 | Chr11 | 68589306 |
| rs10750840 | rs3750965 | 1 | 0.364238 | 3.82E−13 | Chr11 | 68591758 |
| rs3829236 | rs3750965 | 1 | 0.431157 | 1.19E−14 | Chr11 | 68594604 |
| rs3750957 | rs3750965 | 1 | 0.423958 | 3.67E−15 | Chr11 | 68595763 |
| rs3750963 | rs3750965 | 1 | 0.416737 | 2.65E−14 | Chr11 | 68596590 |
| rs3750965 | rs3750965 | 1 | 1 | 0 | Chr11 | 68596736 |
| rs10792020 | rs3750965 | 1 | 0.416737 | 4.84E−15 | Chr11 | 68599067 |
| rs731974 | rs3750965 | 1 | 1 | 1.04E−30 | Chr11 | 68603732 |
| rs896973 | rs3750965 | 1 | 0.413141 | 3.48E−14 | Chr11 | 68608042 |
| rs753559 | rs3750965 | 1 | 0.423958 | 3.67E−15 | Chr11 | 68608181 |
| rs1123665 | rs3750965 | 1 | 0.435357 | 4.92E−15 | Chr11 | 68611773 |
| rs3829241 | rs1011176 | 0.83274 | 0.29446 | 5.87E−10 | Chr11 | 68611939 |
| rs1551306 | rs3750965 | 1 | 0.446154 | 8.77E−16 | Chr11 | 68612059 |
| rs1060435 | rs1011176 | 0.83274 | 0.29446 | 5.87E−10 | Chr11 | 68612171 |
| rs4930265 | rs3750965 | 1 | 0.955022 | 2.70E−28 | Chr11 | 68612530 |
| rs2253658 | rs3750965 | 1 | 0.955022 | 2.70E−28 | Chr11 | 68613380 |
| rs1005858 | rs3750965 | 1 | 0.431157 | 2.09E−15 | Chr11 | 68613492 |
| rs3168115 | rs3750965 | 1 | 0.955022 | 2.70E−28 | Chr11 | 68614666 |
| rs10736671 | rs3750965 | 1 | 0.457672 | 1.23E−14 | Chr11 | 68615483 |
| rs4930651 | rs3750965 | 1 | 0.446154 | 8.77E−16 | Chr11 | 68615606 |
| rs12280942 | rs3750965 | 0.954528 | 0.911124 | 1.75E−25 | Chr11 | 68616259 |
| rs7107680 | rs3750965 | 1 | 0.955022 | 2.70E−28 | Chr11 | 68616470 |
| rs7111999 | rs3750965 | 1 | 0.42758 | 2.77E−15 | Chr11 | 68617234 |
| rs11228490 | rs3750965 | 1 | 0.955022 | 2.70E−28 | Chr11 | 68617448 |
| rs10750842 | rs3750965 | 1 | 0.428299 | 1.14E−14 | Chr11 | 68617474 |
| rs10736672 | rs3750965 | 1 | 0.433725 | 5.98E−14 | Chr11 | 68617905 |
| rs10896422 | rs3750965 | 1 | 0.431157 | 2.09E−15 | Chr11 | 68619900 |
| rs11228494 | rs3750965 | 1 | 0.424132 | 4.87E−15 | Chr11 | 68623147 |
| rs2305498 | rs2305498 | 1 | 1 | 0 | Chr11 | 68623490 |
| rs10896425 | rs3750965 | 1 | 0.431157 | 2.09E−15 | Chr11 | 68624231 |
| rs2123759 | rs3750965 | 1 | 0.471133 | 1.27E−15 | Chr11 | 68624891 |
| rs921675 | rs2305498 | 1 | 1 | 1.32E−26 | Chr11 | 68625610 |
| rs10896426 | rs3750965 | 1 | 0.427257 | 7.91E−15 | Chr11 | 68626499 |
| rs2924536 | rs3750965 | 1 | 0.446154 | 8.77E−16 | Chr11 | 68626681 |
| rs11228498 | rs3750965 | 0.939977 | 0.390913 | 1.27E−12 | Chr11 | 68626874 |
| rs2924533 | rs2305498 | 1 | 0.758031 | 1.09E−19 | Chr11 | 68627577 |
| rs921673 | rs3750965 | 1 | 0.446154 | 2.13E−15 | Chr11 | 68627987 |
| rs921671 | rs3750965 | 1 | 0.446154 | 5.19E−15 | Chr11 | 68628024 |
| rs921670 | rs3750965 | 1 | 0.948906 | 7.25E−26 | Chr11 | 68628215 |
| rs7131509 | rs3750965 | 0.762268 | 0.53295 | 2.06E−13 | Chr11 | 68640132 |
| rs3892895 | rs3750965 | 0.769384 | 0.565932 | 1.97E−14 | Chr11 | 68641331 |
| rs16761 | rs3750965 | 0.824525 | 0.348528 | 1.47E−09 | Chr11 | 68651567 |
| rs10896437 | rs1011176 | 0.919763 | 0.224111 | 1.63E−07 | Chr11 | 68660041 |
| rs11228517 | rs1011176 | 1 | 0.559337 | 1.78E−18 | Chr11 | 68660816 |
| rs10896438 | rs1011176 | 0.925025 | 0.239121 | 4.80E−08 | Chr11 | 68663146 |
| rs7928319 | rs1011176 | 0.918717 | 0.2279 | 2.44E−07 | Chr11 | 68663578 |
| rs2924538 | rs1011176 | 0.922049 | 0.233145 | 2.11E−07 | Chr11 | 68667430 |
| rs3018667 | rs1011176 | 0.94882 | 0.578216 | 9.18E−17 | Chr11 | 68668797 |
| rs11228521 | rs1011176 | 1 | 0.259494 | 7.26E−11 | Chr11 | 68671621 |
| rs3019751 | rs1011176 | 0.516468 | 0.215015 | 1.19E−06 | Chr11 | 68680173 |
| rs3019748 | rs1011176 | 0.944398 | 0.378719 | 2.88E−12 | Chr11 | 68681572 |
| rs1542335 | rs1011176 | 1 | 1 | 6.45E−35 | Chr11 | 68687696 |
| rs1542336 | rs1011176 | 1 | 1 | 6.45E−35 | Chr11 | 68689046 |
| rs1542337 | rs1011176 | 1 | 1 | 1.44E−32 | Chr11 | 68689138 |
| rs7940364 | rs1011176 | 1 | 1 | 6.45E−35 | Chr11 | 68689475 |
| rs1011176 | rs1011176 | 1 | 1 | 0 | Chr11 | 68690473 |
| rs896968 | rs1011176 | 1 | 1 | 2.19E−35 | Chr11 | 68691538 |
| rs12418451 | rs1011176 | 1 | 0.270147 | 4.33E−11 | Chr11 | 68691995 |
| rs11228540 | rs1011176 | 0.959116 | 0.845757 | 2.09E−22 | Chr11 | 68692128 |
| rs4930657 | rs1011176 | 1 | 0.806087 | 1.56E−27 | Chr11 | 68702937 |
| rs12417953 | rs1011176 | 1 | 0.806087 | 1.56E−27 | Chr11 | 68703434 |
| rs7128814 | rs3750965 | 0.664516 | 0.251994 | 6.69E−07 | Chr11 | 68709630 |
| rs11228551 | rs1011176 | 0.852914 | 0.203677 | 5.46E−07 | Chr11 | 68711570 |
| rs12809032 | rs1011176 | 0.917862 | 0.223168 | 2.12E−07 | Chr11 | 68713686 |
| rs4495899 | rs1011176 | 0.906547 | 0.487449 | 6.60E−15 | Chr11 | 68715236 |
| rs4072598 | rs1011176 | 0.906547 | 0.487449 | 6.60E−15 | Chr11 | 68716265 |
| rs10896442 | rs1011176 | 0.906193 | 0.483519 | 9.77E−15 | Chr11 | 68716789 |
| rs11021131 | rs1042602 | 0.7217 | 0.337918 | 1.22E−09 | Chr11 | 88111682 |
| rs11021132 | rs1042602 | 0.483834 | 0.218489 | 8.87E−07 | Chr11 | 88111718 |
| rs12271760 | rs1042602 | 0.715151 | 0.328228 | 3.34E−09 | Chr11 | 88111919 |
| rs1903844 | rs1042602 | 0.630769 | 0.312561 | 4.65E−09 | Chr11 | 88112017 |
| rs1903845 | rs1042602 | 0.632224 | 0.312499 | 6.46E−09 | Chr11 | 88112130 |
| rs6483414 | rs1042602 | 0.575274 | 0.224837 | 6.31E−06 | Chr11 | 88116862 |
| rs12799111 | rs1042602 | 0.636802 | 0.290845 | 5.79E−08 | Chr11 | 88117874 |
| rs12222022 | rs1393350 | 0.564376 | 0.207695 | 0.000012 | Chr11 | 88144702 |
| rs12275597 | rs1042602 | 0.721526 | 0.341443 | 2.49E−09 | Chr11 | 88144728 |
| rs11021284 | rs1042602 | 0.75921 | 0.297708 | 4.25E−07 | Chr11 | 88146242 |
| rs3913310 | rs1393350 | 0.697866 | 0.43836 | 2.07E−10 | Chr11 | 88162391 |
| rs3862367 | rs1042602 | 0.840261 | 0.396104 | 5.44E−11 | Chr11 | 88169104 |
| rs3862368 | rs1042602 | 0.734812 | 0.33805 | 8.52E−09 | Chr11 | 88169122 |
| rs10501687 | rs1393350 | 0.564376 | 0.207695 | 0.000012 | Chr11 | 88171827 |
| rs12787863 | rs1393350 | 0.564376 | 0.207695 | 0.000012 | Chr11 | 88183363 |
| rs12801588 | rs1042602 | 0.7217 | 0.337918 | 1.22E−09 | Chr11 | 88187920 |
| rs11021438 | rs1042602 | 0.7217 | 0.337918 | 1.22E−09 | Chr11 | 88189626 |
| rs12279922 | rs1042602 | 0.723968 | 0.343788 | 1.29E−09 | Chr11 | 88191632 |
| rs2047512 | rs1393350 | 0.564376 | 0.207695 | 0.000012 | Chr11 | 88198124 |
| rs2648640 | rs1393350 | 0.569747 | 0.219451 | 7.20E−06 | Chr11 | 88202558 |
| rs17184781 | rs1393350 | 0.670691 | 0.443193 | 1.99E−10 | Chr11 | 88202679 |
| rs643566 | rs1042602 | 0.748883 | 0.422776 | 6.67E−12 | Chr11 | 88214685 |
| rs1603897 | rs1393350 | 0.563272 | 0.20539 | 0.000013 | Chr11 | 88217878 |
| rs643304 | rs1042602 | 0.713425 | 0.317649 | 4.65E−09 | Chr11 | 88220987 |
| rs316087 | rs1042602 | 0.713425 | 0.317649 | 4.65E−09 | Chr11 | 88223626 |
| rs316086 | rs1042602 | 0.709494 | 0.264768 | 9.46E−07 | Chr11 | 88223808 |
| rs2000819 | rs1393350 | 0.639568 | 0.219927 | 9.79E−06 | Chr11 | 88228117 |
| rs645327 | rs1042602 | 0.7217 | 0.337918 | 1.22E−09 | Chr11 | 88228434 |
| rs652659 | rs1042602 | 0.713425 | 0.317649 | 4.65E−09 | Chr11 | 88239474 |
| rs659197 | rs1042602 | 0.538971 | 0.225386 | 1.07E−06 | Chr11 | 88240462 |
| rs672981 | rs1393350 | 0.569883 | 0.211768 | 6.29E−06 | Chr11 | 88262761 |
| rs316096 | rs1042602 | 0.756666 | 0.22838 | 2.83E−07 | Chr11 | 88268603 |
| rs594561 | rs1042602 | 0.753507 | 0.222767 | 4.43E−07 | Chr11 | 88272620 |
| rs598758 | rs1393350 | 0.575785 | 0.225686 | 3.33E−06 | Chr11 | 88274783 |
| rs679333 | rs1393350 | 0.575785 | 0.225686 | 3.33E−06 | Chr11 | 88282234 |
| rs640211 | rs1393350 | 0.581535 | 0.240563 | 1.70E−06 | Chr11 | 88290695 |
| rs621230 | rs1042602 | 0.748545 | 0.216268 | 7.43E−07 | Chr11 | 88293840 |
| rs596370 | rs1393350 | 0.622186 | 0.222919 | 1.67E−06 | Chr11 | 88301450 |
| rs1289097 | rs1393350 | 0.621225 | 0.220545 | 1.92E−06 | Chr11 | 88301873 |
| rs493306 | rs1393350 | 0.580457 | 0.245928 | 2.40E−06 | Chr11 | 88302847 |
| rs538601 | rs1042602 | 0.740993 | 0.208258 | 1.40E−06 | Chr11 | 88305340 |
| rs573060 | rs1393350 | 0.581535 | 0.240563 | 1.70E−06 | Chr11 | 88312097 |
| rs633748 | rs1393350 | 0.581535 | 0.240563 | 1.70E−06 | Chr11 | 88312212 |
| rs655683 | rs1393350 | 0.557445 | 0.271257 | 4.45E−06 | Chr11 | 88316502 |
| rs591799 | rs1042602 | 0.805873 | 0.23194 | 1.11E−07 | Chr11 | 88319446 |
| rs561940 | rs1042602 | 0.801914 | 0.230237 | 1.57E−07 | Chr11 | 88325812 |
| rs624913 | rs1042602 | 0.755254 | 0.22255 | 3.16E−07 | Chr11 | 88326326 |
| rs598134 | rs1393350 | 0.605054 | 0.229547 | 8.04E−06 | Chr11 | 88327228 |
| rs597462 | rs1042602 | 0.855034 | 0.399331 | 2.15E−12 | Chr11 | 88327881 |
| rs627387 | rs1042602 | 0.855034 | 0.399331 | 2.15E−12 | Chr11 | 88333720 |
| rs496939 | rs1393350 | 0.669509 | 0.258119 | 3.06E−06 | Chr11 | 88334118 |
| rs651890 | rs1393350 | 0.864137 | 0.338538 | 9.77E−10 | Chr11 | 88335031 |
| rs567990 | rs1042602 | 0.900676 | 0.430383 | 1.80E−13 | Chr11 | 88343740 |
| rs534815 | rs1042602 | 0.899504 | 0.43379 | 4.26E−13 | Chr11 | 88344566 |
| rs524874 | rs1042602 | 0.895526 | 0.381889 | 2.14E−12 | Chr11 | 88352031 |
| rs10765819 | rs1042602 | 0.901532 | 0.429097 | 1.49E−13 | Chr11 | 88370876 |
| rs10765820 | rs1042602 | 0.901532 | 0.429097 | 1.49E−13 | Chr11 | 88373771 |
| rs12283766 | rs1393350 | 0.864542 | 0.341485 | 8.06E−10 | Chr11 | 88379580 |
| rs7120151 | rs1393350 | 0.861272 | 0.552073 | 9.41E−13 | Chr11 | 88380027 |
| rs7127754 | rs1393350 | 0.866448 | 0.356018 | 3.93E−10 | Chr11 | 88381038 |
| rs7929619 | rs1393350 | 0.866075 | 0.353077 | 4.76E−10 | Chr11 | 88384064 |
| rs7932640 | rs1393350 | 0.866075 | 0.353077 | 4.76E−10 | Chr11 | 88384073 |
| rs10830200 | rs1042602 | 0.896847 | 0.396503 | 1.43E−12 | Chr11 | 88385087 |
| rs10765183 | rs1393350 | 0.866448 | 0.356018 | 3.93E−10 | Chr11 | 88385387 |
| rs10501696 | rs1042602 | 0.899244 | 0.412571 | 4.70E−13 | Chr11 | 88387810 |
| rs2226563 | rs1393350 | 0.866448 | 0.356018 | 3.93E−10 | Chr11 | 88388720 |
| rs7126679 | rs1393350 | 0.931282 | 0.379009 | 3.40E−11 | Chr11 | 88393493 |
| rs4505064 | rs1393350 | 0.861585 | 0.361618 | 7.77E−10 | Chr11 | 88394996 |
| rs10430814 | rs1393350 | 0.868302 | 0.371294 | 1.86E−10 | Chr11 | 88397245 |
| rs7125164 | rs1042602 | 0.899244 | 0.412571 | 4.70E−13 | Chr11 | 88397598 |
| rs10430815 | rs1393350 | 0.866838 | 0.370043 | 3.69E−10 | Chr11 | 88398369 |
| rs12224116 | rs1393350 | 0.868302 | 0.371294 | 1.86E−10 | Chr11 | 88400454 |
| rs11018434 | rs1042602 | 0.868703 | 0.471653 | 1.83E−14 | Chr11 | 88405427 |
| rs17791976 | rs1393350 | 0.940708 | 0.57703 | 6.79E−16 | Chr11 | 88408490 |
| rs7931721 | rs1393350 | 0.939328 | 0.529188 | 6.19E−15 | Chr11 | 88419424 |
| rs11018440 | rs1393350 | 0.940708 | 0.57703 | 6.79E−16 | Chr11 | 88426718 |
| rs11018441 | rs1393350 | 1 | 0.625117 | 7.04E−18 | Chr11 | 88426947 |
| rs10830204 | rs1042602 | 0.867031 | 0.468103 | 3.09E−14 | Chr11 | 88427192 |
| rs11018449 | rs1393350 | 0.935726 | 0.596103 | 5.55E−14 | Chr11 | 88437034 |
| rs477424 | rs1393350 | 1 | 0.424598 | 2.80E−14 | Chr11 | 88441929 |
| rs7929744 | rs1042602 | 0.858129 | 0.45556 | 5.31E−13 | Chr11 | 88444332 |
| rs7127487 | rs1393350 | 0.932877 | 0.403925 | 5.42E−12 | Chr11 | 88454518 |
| rs10830206 | rs1393350 | 0.934921 | 0.441478 | 8.34E−13 | Chr11 | 88455785 |
| rs4121738 | rs1393350 | 0.933415 | 0.413176 | 2.93E−12 | Chr11 | 88456186 |
| rs11018463 | rs1393350 | 0.940708 | 0.57703 | 6.79E−16 | Chr11 | 88459390 |
| rs11018464 | rs1393350 | 0.938045 | 0.574881 | 2.49E−15 | Chr11 | 88460762 |
| rs3921012 | rs1393350 | 0.934329 | 0.429908 | 1.29E−12 | Chr11 | 88465991 |
| rs7944714 | rs1393350 | 0.93324 | 0.410128 | 3.59E−12 | Chr11 | 88470143 |
| rs10765186 | rs1042602 | 0.868676 | 0.488684 | 8.91E−15 | Chr11 | 88470985 |
| rs9665831 | rs1393350 | 0.931151 | 0.424873 | 4.75E−12 | Chr11 | 88473805 |
| rs1942497 | rs1393350 | 0.93539 | 0.504772 | 5.70E−14 | Chr11 | 88481107 |
| rs2156123 | rs1393350 | 0.933415 | 0.413176 | 2.93E−12 | Chr11 | 88488507 |
| rs7930256 | rs1393350 | 0.936617 | 0.466612 | 1.14E−13 | Chr11 | 88489082 |
| rs4420272 | rs1393350 | 0.936617 | 0.466612 | 1.14E−13 | Chr11 | 88490030 |
| rs7480884 | rs1393350 | 0.933735 | 0.477647 | 1.49E−12 | Chr11 | 88491615 |
| rs12363323 | rs1393350 | 0.940708 | 0.57703 | 6.79E−16 | Chr11 | 88495940 |
| rs1942486 | rs1393350 | 0.938045 | 0.574881 | 2.49E−15 | Chr11 | 88496430 |
| rs10830216 | rs1393350 | 0.934329 | 0.429908 | 1.29E−12 | Chr11 | 88498045 |
| rs11018488 | rs1042602 | 0.921636 | 0.688471 | 2.31E−21 | Chr11 | 88501238 |
| rs17792911 | rs1393350 | 0.940417 | 0.566243 | 1.55E−15 | Chr11 | 88502470 |
| rs4121729 | rs1393350 | 0.93324 | 0.410128 | 3.59E−12 | Chr11 | 88502788 |
| rs10830219 | rs1393350 | 0.940026 | 0.552384 | 2.11E−15 | Chr11 | 88512157 |
| rs10830220 | rs1042602 | 0.911709 | 0.515477 | 4.66E−16 | Chr11 | 88513800 |
| rs4121744 | rs1042602 | 0.908475 | 0.482334 | 3.92E−15 | Chr11 | 88514617 |
| rs10830228 | rs1042602 | 0.947934 | 0.427895 | 3.19E−14 | Chr11 | 88530762 |
| rs10830231 | rs1042602 | 0.945368 | 0.396826 | 2.92E−13 | Chr11 | 88535036 |
| rs7127661 | rs1042602 | 0.946682 | 0.41215 | 9.80E−14 | Chr11 | 88536257 |
| rs10830236 | rs1393350 | 0.94179 | 0.630936 | 1.13E−16 | Chr11 | 88540464 |
| rs949537 | rs1042602 | 0.946682 | 0.41215 | 9.80E−14 | Chr11 | 88542478 |
| rs5021654 | rs1393350 | 1 | 0.511666 | 3.89E−16 | Chr11 | 88550237 |
| rs1042602 | rs1042602 | 1 | 1 | 0 | Chr11 | 88551344 |
| rs12270717 | rs1393350 | 1 | 0.948454 | 1.66E−25 | Chr11 | 88551838 |
| rs621313 | rs1042602 | 1 | 0.523013 | 2.61E−19 | Chr11 | 88553311 |
| rs7129973 | rs1393350 | 1 | 0.531903 | 1.48E−16 | Chr11 | 88555218 |
| rs11018525 | rs1393350 | 1 | 0.528884 | 1.84E−16 | Chr11 | 88559553 |
| rs17793678 | rs1393350 | 1 | 1 | 3.10E−27 | Chr11 | 88561172 |
| rs594647 | rs1393350 | 1 | 0.464707 | 1.38E−14 | Chr11 | 88561205 |
| rs10765196 | rs1393350 | 1 | 1 | 3.10E−27 | Chr11 | 88564890 |
| rs10765197 | rs1393350 | 1 | 0.528884 | 1.84E−16 | Chr11 | 88564976 |
| rs7123654 | rs1393350 | 1 | 0.525826 | 2.29E−16 | Chr11 | 88565603 |
| rs11018528 | rs1393350 | 1 | 1 | 2.50E−27 | Chr11 | 88570025 |
| rs12791412 | rs1393350 | 1 | 0.948454 | 1.66E−25 | Chr11 | 88570229 |
| rs12789914 | rs1393350 | 0.945 | 0.804644 | 1.44E−20 | Chr11 | 88570555 |
| rs7107143 | rs1393350 | 1 | 0.857256 | 2.98E−23 | Chr11 | 88571135 |
| rs574028 | rs1042602 | 1 | 0.487805 | 3.31E−18 | Chr11 | 88572898 |
| rs2000553 | rs1393350 | 1 | 0.531903 | 1.48E−16 | Chr11 | 88575655 |
| rs11018541 | rs1393350 | 1 | 0.511666 | 3.89E−16 | Chr11 | 88599795 |
| rs10765198 | rs1393350 | 1 | 1 | 3.13E−26 | Chr11 | 88609422 |
| rs7358418 | rs1393350 | 1 | 0.900083 | 3.51E−24 | Chr11 | 88609786 |
| rs10765200 | rs1393350 | 1 | 0.946333 | 3.92E−25 | Chr11 | 88611332 |
| rs10765201 | rs1393350 | 1 | 0.946879 | 3.16E−25 | Chr11 | 88611352 |
| rs4396293 | rs1393350 | 1 | 0.522727 | 2.85E−16 | Chr11 | 88615761 |
| rs2186640 | rs1393350 | 1 | 0.511666 | 3.89E−16 | Chr11 | 88615811 |
| rs10501698 | rs1393350 | 0.937871 | 0.741926 | 2.65E−17 | Chr11 | 88617012 |
| rs10830250 | rs1393350 | 1 | 0.632135 | 5.13E−18 | Chr11 | 88617255 |
| rs7924589 | rs1393350 | 1 | 0.724928 | 1.08E−17 | Chr11 | 88617956 |
| rs4121401 | rs1393350 | 1 | 0.492466 | 9.81E−16 | Chr11 | 88619494 |
| rs10741305 | rs1042602 | 0.810574 | 0.396938 | 9.29E−12 | Chr11 | 88622366 |
| rs591260 | rs1042602 | 0.776064 | 0.377744 | 1.85E−11 | Chr11 | 88642214 |
| rs1847134 | rs1393350 | 1 | 0.779087 | 6.27E−21 | Chr11 | 88644901 |
| rs1393350 | rs1393350 | 1 | 1 | 0 | Chr11 | 88650694 |
| rs1827430 | rs1393350 | 1 | 0.471092 | 2.95E−15 | Chr11 | 88658088 |
| rs3900053 | rs1393350 | 0.921805 | 0.736136 | 2.29E−14 | Chr11 | 88660713 |
| rs1847142 | rs1393350 | 1 | 0.8151 | 1.29E−21 | Chr11 | 88661222 |
| rs501301 | rs1042602 | 0.784503 | 0.397736 | 3.43E−12 | Chr11 | 88662321 |
| rs4121403 | rs1393350 | 0.780076 | 0.577152 | 4.06E−13 | Chr11 | 88664103 |
| rs10830253 | rs1393350 | 1 | 0.816724 | 2.31E−22 | Chr11 | 88667691 |
| rs7951935 | rs1393350 | 1 | 0.433962 | 2.17E−13 | Chr11 | 88670047 |
| rs1502259 | rs1042602 | 0.850344 | 0.402238 | 1.04E−11 | Chr11 | 88675893 |
| rs1847140 | rs1393350 | 0.780076 | 0.577152 | 4.06E−13 | Chr11 | 88676712 |
| rs1806319 | rs1393350 | 1 | 0.511666 | 3.89E−16 | Chr11 | 88677584 |
| rs4106039 | rs1393350 | 0.756902 | 0.417805 | 4.24E−09 | Chr11 | 88680791 |
| rs4106040 | rs1393350 | 0.795779 | 0.410735 | 1.45E−08 | Chr11 | 88680802 |
| rs10830256 | rs1042602 | 0.815842 | 0.388986 | 7.59E−12 | Chr11 | 88685204 |
| rs317185 | rs1042602 | 0.616799 | 0.208981 | 1.45E−06 | Chr11 | 88735421 |
| rs317166 | rs1042602 | 0.678018 | 0.279229 | 3.34E−08 | Chr11 | 88747642 |
| rs317174 | rs1042602 | 0.624896 | 0.228211 | 1.13E−06 | Chr11 | 88750955 |
| rs317175 | rs1042602 | 0.657763 | 0.246985 | 3.22E−07 | Chr11 | 88751078 |
| rs317169 | rs1042602 | 0.63413 | 0.235006 | 3.31E−07 | Chr11 | 88756036 |
| rs317155 | rs1042602 | 0.626427 | 0.221725 | 7.75E−07 | Chr11 | 88766735 |
| rs488342 | rs1042602 | 0.63022 | 0.224064 | 2.00E−06 | Chr11 | 88774816 |
| rs546460 | rs1042602 | 0.618373 | 0.208866 | 1.77E−06 | Chr11 | 88781154 |
| rs317126 | rs1042602 | 0.566534 | 0.207424 | 2.26E−06 | Chr11 | 88793423 |
| rs317127 | rs1042602 | 0.564737 | 0.212306 | 2.15E−06 | Chr11 | 88796019 |
| rs317129 | rs1042602 | 0.566534 | 0.207424 | 2.26E−06 | Chr11 | 88796682 |
| rs579497 | rs1042602 | 0.564331 | 0.208171 | 3.61E−06 | Chr11 | 88797304 |
| rs317147 | rs1042602 | 0.586912 | 0.201311 | 3.09E−06 | Chr11 | 88807317 |
| rs319030 | rs1042602 | 0.586912 | 0.201311 | 3.09E−06 | Chr11 | 88815035 |
| rs11104703 | rs4842602 | 0.723061 | 0.211953 | 0.00007 | Chr12 | 86903919 |
| rs11104732 | rs3782181 | 1 | 0.40249 | 5.91E−10 | Chr12 | 87002119 |
| rs11104738 | rs1022034 | 0.633017 | 0.245894 | 0.000211 | Chr12 | 87024978 |
| rs17335988 | rs3782181 | 0.880788 | 0.347736 | 1.75E−07 | Chr12 | 87067911 |
| rs17421009 | rs4842602 | 0.738452 | 0.227633 | 8.80E−06 | Chr12 | 87104405 |
| rs7314836 | rs4842602 | 0.709739 | 0.307673 | 1.35E−06 | Chr12 | 87188705 |
| rs11104784 | rs4842602 | 0.709952 | 0.326251 | 1.15E−06 | Chr12 | 87194765 |
| rs7966318 | rs4842602 | 0.638264 | 0.271808 | 5.26E−06 | Chr12 | 87201455 |
| rs11104789 | rs1022034 | 1 | 0.26893 | 0.000083 | Chr12 | 87202932 |
| rs11104790 | rs1022034 | 0.781044 | 0.279839 | 0.000056 | Chr12 | 87203376 |
| rs2216153 | rs4842602 | 0.861062 | 0.407786 | 2.94E−10 | Chr12 | 87206108 |
| rs10858701 | rs4842602 | 0.790447 | 0.391358 | 3.69E−07 | Chr12 | 87206552 |
| rs11104794 | rs4842602 | 0.884356 | 0.396312 | 4.79E−08 | Chr12 | 87209007 |
| rs11104795 | rs4842602 | 0.792256 | 0.350052 | 2.97E−07 | Chr12 | 87209680 |
| rs7313206 | rs4842602 | 1 | 0.428571 | 1.17E−09 | Chr12 | 87209890 |
| rs11832237 | rs4842602 | 1 | 0.405405 | 1.73E−09 | Chr12 | 87211933 |
| rs7976732 | rs4842602 | 0.795794 | 0.351826 | 2.49E−07 | Chr12 | 87214920 |
| rs11104797 | rs4842602 | 1 | 0.405405 | 1.60E−09 | Chr12 | 87215071 |
| rs10777100 | rs4842602 | 0.88042 | 0.378343 | 3.39E−07 | Chr12 | 87215720 |
| rs10777101 | rs4842602 | 0.797541 | 0.380043 | 3.05E−07 | Chr12 | 87216251 |
| rs11104800 | rs1022034 | 1 | 0.26893 | 0.000083 | Chr12 | 87217192 |
| rs9739175 | rs4842602 | 0.795794 | 0.351826 | 2.49E−07 | Chr12 | 87217859 |
| rs9738049 | rs4842602 | 0.795794 | 0.351826 | 2.49E−07 | Chr12 | 87218067 |
| rs11104801 | rs4842602 | 0.79404 | 0.350947 | 2.72E−07 | Chr12 | 87218305 |
| rs10858704 | rs4842602 | 0.795794 | 0.351826 | 2.49E−07 | Chr12 | 87218518 |
| rs11104805 | rs4842602 | 0.784045 | 0.325522 | 1.44E−06 | Chr12 | 87219905 |
| rs11104806 | rs4842602 | 0.79404 | 0.350947 | 2.72E−07 | Chr12 | 87220116 |
| rs7310550 | rs4842602 | 0.79404 | 0.350947 | 2.72E−07 | Chr12 | 87221068 |
| rs10858705 | rs4842602 | 0.851216 | 0.335694 | 1.37E−08 | Chr12 | 87221158 |
| rs10777103 | rs4842602 | 0.795794 | 0.351826 | 2.49E−07 | Chr12 | 87221489 |
| rs11104807 | rs4842602 | 0.795794 | 0.351826 | 2.49E−07 | Chr12 | 87222772 |
| rs10858706 | rs4842602 | 0.788171 | 0.347161 | 6.50E−07 | Chr12 | 87222983 |
| rs12370662 | rs4842602 | 1 | 0.405405 | 1.73E−09 | Chr12 | 87223329 |
| rs7131695 | rs4842602 | 0.792256 | 0.350052 | 2.97E−07 | Chr12 | 87224098 |
| rs11104808 | rs4842602 | 0.79404 | 0.350947 | 2.72E−07 | Chr12 | 87224214 |
| rs11104809 | rs4842602 | 0.79404 | 0.350947 | 2.72E−07 | Chr12 | 87224601 |
| rs11104810 | rs4842602 | 1 | 0.405405 | 1.73E−09 | Chr12 | 87224707 |
| rs12370518 | rs4842602 | 1 | 0.405405 | 1.60E−09 | Chr12 | 87226318 |
| rs7974675 | rs4842602 | 0.795794 | 0.351826 | 2.49E−07 | Chr12 | 87226615 |
| rs7974819 | rs4842602 | 0.795794 | 0.351826 | 2.49E−07 | Chr12 | 87226720 |
| rs7978158 | rs4842602 | 0.79404 | 0.350947 | 2.72E−07 | Chr12 | 87227161 |
| rs12369673 | rs4842602 | 1 | 0.405405 | 1.60E−09 | Chr12 | 87228166 |
| rs17422027 | rs4842602 | 1 | 0.405405 | 1.60E−09 | Chr12 | 87228421 |
| rs11104811 | rs4842602 | 0.795794 | 0.351826 | 2.49E−07 | Chr12 | 87228581 |
| rs11104812 | rs4842602 | 1 | 0.405405 | 1.60E−09 | Chr12 | 87228741 |
| rs10858708 | rs4842602 | 0.793641 | 0.349925 | 4.99E−07 | Chr12 | 87228817 |
| rs10858709 | rs4842602 | 0.804702 | 0.384671 | 1.14E−07 | Chr12 | 87229040 |
| rs11104813 | rs4842602 | 1 | 0.405405 | 1.60E−09 | Chr12 | 87229334 |
| rs7962013 | rs4842602 | 0.795794 | 0.351826 | 2.49E−07 | Chr12 | 87230111 |
| rs10858710 | rs4842602 | 0.795794 | 0.351826 | 2.49E−07 | Chr12 | 87230881 |
| rs11104815 | rs4842602 | 1 | 0.405405 | 1.88E−09 | Chr12 | 87231713 |
| rs10858711 | rs4842602 | 0.79404 | 0.350947 | 2.72E−07 | Chr12 | 87232099 |
| rs7977272 | rs4842602 | 0.795794 | 0.351826 | 2.49E−07 | Chr12 | 87232391 |
| rs7977437 | rs4842602 | 0.802985 | 0.383753 | 1.26E−07 | Chr12 | 87232620 |
| rs7964336 | rs4842602 | 0.795794 | 0.351826 | 2.49E−07 | Chr12 | 87232677 |
| rs12318457 | rs4842602 | 0.79404 | 0.350947 | 2.72E−07 | Chr12 | 87234155 |
| rs7980026 | rs4842602 | 0.795794 | 0.351826 | 2.49E−07 | Chr12 | 87236379 |
| rs11104818 | rs4842602 | 0.795794 | 0.351826 | 2.49E−07 | Chr12 | 87237037 |
| rs12367954 | rs4842602 | 1 | 0.405405 | 1.60E−09 | Chr12 | 87240462 |
| rs1014300 | rs4842602 | 0.879998 | 0.774397 | 2.97E−17 | Chr12 | 87241535 |
| rs17422329 | rs4842602 | 1 | 0.405405 | 1.60E−09 | Chr12 | 87245597 |
| rs2041857 | rs4842602 | 1 | 0.357143 | 1.65E−08 | Chr12 | 87248876 |
| rs2041859 | rs4842602 | 1 | 1 | 2.51E−25 | Chr12 | 87249060 |
| rs2897727 | rs4842602 | 1 | 0.357143 | 1.65E−08 | Chr12 | 87250164 |
| rs10745476 | rs4842602 | 1 | 0.357143 | 1.65E−08 | Chr12 | 87254582 |
| rs4842602 | rs4842602 | 1 | 1 | 0 | Chr12 | 87256716 |
| rs2193025 | rs4842602 | 1 | 0.357143 | 1.65E−08 | Chr12 | 87258529 |
| rs2216155 | rs12821256 | 0.712699 | 0.359656 | 1.31E−06 | Chr12 | 87258841 |
| rs10777105 | rs4842602 | 1 | 0.357143 | 1.65E−08 | Chr12 | 87259825 |
| rs2407657 | rs4842602 | 1 | 0.357143 | 1.65E−08 | Chr12 | 87260566 |
| rs7136832 | rs4842602 | 1 | 0.357143 | 1.65E−08 | Chr12 | 87260710 |
| rs7306001 | rs4842602 | 1 | 0.357143 | 1.65E−08 | Chr12 | 87260732 |
| rs2111040 | rs4842602 | 1 | 0.555556 | 8.78E−13 | Chr12 | 87262511 |
| rs12424030 | rs12821256 | 0.775492 | 0.359061 | 1.32E−06 | Chr12 | 87262822 |
| rs2111041 | rs4842602 | 1 | 0.357143 | 1.65E−08 | Chr12 | 87263566 |
| rs7965472 | rs4842602 | 1 | 0.357143 | 1.65E−08 | Chr12 | 87264258 |
| rs10777106 | rs4842602 | 1 | 0.357143 | 1.65E−08 | Chr12 | 87264732 |
| rs10745477 | rs4842602 | 0.861266 | 0.301098 | 1.65E−06 | Chr12 | 87265181 |
| rs10858713 | rs4842602 | 1 | 0.357143 | 1.65E−08 | Chr12 | 87267586 |
| rs4842603 | rs4842602 | 1 | 0.357143 | 1.65E−08 | Chr12 | 87268544 |
| rs10858714 | rs4842602 | 1 | 0.357143 | 1.78E−08 | Chr12 | 87270340 |
| rs10745478 | rs4842602 | 1 | 0.357143 | 1.65E−08 | Chr12 | 87270942 |
| rs2193027 | rs4842602 | 1 | 1 | 3.02E−25 | Chr12 | 87275976 |
| rs12423928 | rs12821256 | 0.731064 | 0.393422 | 2.90E−08 | Chr12 | 87278286 |
| rs10858715 | rs4842602 | 1 | 0.357143 | 1.65E−08 | Chr12 | 87279708 |
| rs17338168 | rs4842602 | 1 | 0.555556 | 8.78E−13 | Chr12 | 87280732 |
| rs6538144 | rs4842602 | 1 | 0.257732 | 5.33E−06 | Chr12 | 87283093 |
| rs10777109 | rs4842602 | 1 | 0.357143 | 1.65E−08 | Chr12 | 87290698 |
| rs10745479 | rs4842602 | 1 | 0.357143 | 1.65E−08 | Chr12 | 87291483 |
| rs2407653 | rs4842602 | 1 | 0.287313 | 1.10E−06 | Chr12 | 87292097 |
| rs10777110 | rs4842602 | 1 | 0.357143 | 1.65E−08 | Chr12 | 87293154 |
| rs10858718 | rs4842602 | 1 | 0.357143 | 1.65E−08 | Chr12 | 87293574 |
| rs2407649 | rs4842602 | 1 | 0.357143 | 1.65E−08 | Chr12 | 87295390 |
| rs2160432 | rs4842602 | 1 | 0.357143 | 1.65E−08 | Chr12 | 87296520 |
| rs2111038 | rs4842602 | 1 | 0.357143 | 1.65E−08 | Chr12 | 87296649 |
| rs4842604 | rs4842602 | 1 | 0.357143 | 1.65E−08 | Chr12 | 87297081 |
| rs12422914 | rs12821256 | 0.805788 | 0.437107 | 5.89E−09 | Chr12 | 87330195 |
| rs4842610 | rs4842602 | 1 | 0.466667 | 1.86E−14 | Chr12 | 87335820 |
| rs4842611 | rs12821256 | 0.498813 | 0.202896 | 0.000048 | Chr12 | 87335930 |
| rs11104860 | rs995030 | 1 | 0.668966 | 8.45E−16 | Chr12 | 87339118 |
| rs6538148 | rs4842602 | 1 | 0.457627 | 1.09E−13 | Chr12 | 87342610 |
| rs7484757 | rs995030 | 1 | 0.668966 | 8.45E−16 | Chr12 | 87344571 |
| rs10858729 | rs995030 | 1 | 0.668966 | 8.45E−16 | Chr12 | 87345297 |
| rs10777115 | rs995030 | 1 | 0.668966 | 8.45E−16 | Chr12 | 87349735 |
| rs11104865 | rs4842602 | 1 | 0.555556 | 8.78E−13 | Chr12 | 87351066 |
| rs11104867 | rs995030 | 1 | 0.668966 | 9.68E−16 | Chr12 | 87351984 |
| rs11104868 | rs995030 | 1 | 0.668966 | 8.45E−16 | Chr12 | 87352573 |
| rs11104870 | rs4842602 | 1 | 0.476744 | 1.40E−14 | Chr12 | 87353425 |
| rs7487416 | rs995030 | 1 | 0.668966 | 8.45E−16 | Chr12 | 87353650 |
| rs7960514 | rs995030 | 1 | 0.707828 | 9.20E−16 | Chr12 | 87354466 |
| rs7973853 | rs995030 | 1 | 0.63608 | 8.38E−14 | Chr12 | 87354639 |
| rs10858731 | rs995030 | 1 | 0.668966 | 8.45E−16 | Chr12 | 87355810 |
| rs10858733 | rs995030 | 0.885672 | 0.423649 | 1.73E−07 | Chr12 | 87359768 |
| rs7487564 | rs995030 | 1 | 0.668966 | 1.27E−15 | Chr12 | 87362094 |
| rs10858736 | rs995030 | 1 | 0.661882 | 8.45E−15 | Chr12 | 87362110 |
| rs7484884 | rs995030 | 1 | 0.668966 | 1.11E−15 | Chr12 | 87362214 |
| rs11104878 | rs995030 | 1 | 0.668966 | 9.68E−16 | Chr12 | 87363022 |
| rs10777119 | rs995030 | 1 | 0.667145 | 3.97E−15 | Chr12 | 87363876 |
| rs7487130 | rs995030 | 1 | 0.63608 | 4.99E−14 | Chr12 | 87365563 |
| rs10777121 | rs995030 | 1 | 0.668966 | 8.45E−16 | Chr12 | 87367896 |
| rs7979666 | rs995030 | 1 | 0.668966 | 9.68E−16 | Chr12 | 87367958 |
| rs10858741 | rs995030 | 1 | 0.668966 | 8.45E−16 | Chr12 | 87369549 |
| rs4842620 | rs995030 | 1 | 0.668966 | 8.45E−16 | Chr12 | 87370350 |
| rs7974506 | rs1022034 | 1 | 0.562842 | 8.10E−09 | Chr12 | 87372137 |
| rs1162374 | rs995030 | 1 | 0.668966 | 8.45E−16 | Chr12 | 87375674 |
| rs17423182 | rs4842602 | 1 | 0.493409 | 2.18E−10 | Chr12 | 87376155 |
| rs10777123 | rs995030 | 1 | 1 | 9.13E−26 | Chr12 | 87388821 |
| rs11104895 | rs4842602 | 1 | 0.555556 | 8.78E−13 | Chr12 | 87389137 |
| rs11104896 | rs4842602 | 1 | 0.555556 | 8.78E−13 | Chr12 | 87389372 |
| rs1508598 | rs1022034 | 1 | 0.885387 | 6.62E−14 | Chr12 | 87390053 |
| rs4842621 | rs4842602 | 1 | 0.555556 | 8.78E−13 | Chr12 | 87391827 |
| rs11104898 | rs4842602 | 1 | 0.487805 | 9.82E−10 | Chr12 | 87392708 |
| rs11104901 | rs4842602 | 1 | 0.555556 | 8.78E−13 | Chr12 | 87398310 |
| rs11104902 | rs4842602 | 1 | 0.555556 | 8.78E−13 | Chr12 | 87398537 |
| rs11104903 | rs4842602 | 1 | 0.555556 | 8.78E−13 | Chr12 | 87407546 |
| rs12344 | rs4842602 | 1 | 0.555556 | 8.78E−13 | Chr12 | 87411094 |
| rs11104904 | rs4842602 | 1 | 0.25 | 0.000026 | Chr12 | 87411855 |
| rs1508594 | rs1022034 | 1 | 0.298866 | 0.000059 | Chr12 | 87412022 |
| rs1388789 | rs4842602 | 1 | 0.510204 | 3.92E−11 | Chr12 | 87412346 |
| rs4842625 | rs995030 | 1 | 1 | 9.13E−26 | Chr12 | 87412378 |
| rs1907699 | rs995030 | 1 | 1 | 7.54E−26 | Chr12 | 87414108 |
| rs995029 | rs1022034 | 0.889819 | 0.786729 | 3.63E−12 | Chr12 | 87414652 |
| rs11104905 | rs4842602 | 0.906428 | 0.49911 | 2.45E−10 | Chr12 | 87414712 |
| rs995030 | rs995030 | 1 | 1 | 0 | Chr12 | 87414802 |
| rs906639 | rs4842602 | 1 | 0.555556 | 8.78E−13 | Chr12 | 87415584 |
| rs11104906 | rs4842602 | 0.703538 | 0.439101 | 1.03E−08 | Chr12 | 87415907 |
| rs11104907 | rs4842602 | 1 | 0.555556 | 8.78E−13 | Chr12 | 87417390 |
| rs10506953 | rs4842602 | 1 | 0.555556 | 8.78E−13 | Chr12 | 87424377 |
| rs1162372 | rs1022034 | 1 | 0.587261 | 4.62E−08 | Chr12 | 87428062 |
| rs11104911 | rs4842602 | 1 | 0.555556 | 8.78E−13 | Chr12 | 87428895 |
| rs1355062 | rs995030 | 1 | 1 | 1.11E−25 | Chr12 | 87430920 |
| rs4284478 | rs995030 | 1 | 1 | 7.52E−26 | Chr12 | 87431694 |
| rs2291557 | rs4842602 | 1 | 0.588235 | 4.08E−13 | Chr12 | 87434438 |
| rs11104913 | rs4842602 | 1 | 0.555556 | 8.78E−13 | Chr12 | 87435153 |
| rs11104914 | rs4842602 | 1 | 0.555556 | 8.78E−13 | Chr12 | 87437548 |
| rs11104915 | rs4842602 | 1 | 0.504587 | 1.30E−11 | Chr12 | 87437735 |
| rs10777125 | rs1022034 | 1 | 0.885387 | 6.11E−14 | Chr12 | 87439966 |
| rs4842627 | rs4842602 | 1 | 0.555556 | 1.21E−12 | Chr12 | 87442571 |
| rs3782170 | rs4842602 | 1 | 0.555556 | 1.09E−12 | Chr12 | 87442714 |
| rs3782171 | rs4842602 | 0.836227 | 0.522186 | 4.20E−10 | Chr12 | 87442758 |
| rs3782172 | rs4842602 | 1 | 0.555556 | 1.09E−12 | Chr12 | 87442864 |
| rs1022034 | rs1022034 | 1 | 1 | 0 | Chr12 | 87442874 |
| rs17424193 | rs4842602 | 1 | 0.588235 | 4.57E−13 | Chr12 | 87442980 |
| rs11104920 | rs4842602 | 1 | 0.533981 | 6.78E−12 | Chr12 | 87443450 |
| rs11104921 | rs4842602 | 1 | 0.555556 | 1.09E−12 | Chr12 | 87443614 |
| rs12366295 | rs4842602 | 1 | 0.555556 | 8.78E−13 | Chr12 | 87444150 |
| rs11104922 | rs4842602 | 1 | 0.555556 | 9.78E−13 | Chr12 | 87445085 |
| rs11104924 | rs4842602 | 1 | 0.555556 | 1.09E−12 | Chr12 | 87445347 |
| rs11104928 | rs4842602 | 1 | 0.555556 | 9.78E−13 | Chr12 | 87445548 |
| rs1492356 | rs4842602 | 1 | 0.555556 | 1.09E−12 | Chr12 | 87446092 |
| rs4842476 | rs4842602 | 1 | 0.555556 | 1.09E−12 | Chr12 | 87447105 |
| rs7969188 | rs1022034 | 1 | 0.887081 | 6.67E−14 | Chr12 | 87448198 |
| rs10506954 | rs4842602 | 1 | 0.555556 | 8.78E−13 | Chr12 | 87448883 |
| rs11104930 | rs4842602 | 0.826192 | 0.450769 | 2.16E−09 | Chr12 | 87449203 |
| rs11104931 | rs4842602 | 1 | 0.555556 | 9.78E−13 | Chr12 | 87449344 |
| rs11104932 | rs4842602 | 1 | 0.555556 | 1.21E−12 | Chr12 | 87449568 |
| rs1492347 | rs1022034 | 1 | 0.885387 | 6.11E−14 | Chr12 | 87450104 |
| rs4842477 | rs4842602 | 1 | 0.403627 | 1.91E−08 | Chr12 | 87450203 |
| rs4842631 | rs4842602 | 1 | 0.4 | 8.20E−09 | Chr12 | 87450918 |
| rs11104934 | rs4842602 | 0.905544 | 0.498577 | 2.72E−10 | Chr12 | 87451446 |
| rs10506955 | rs4842602 | 1 | 0.555556 | 8.78E−13 | Chr12 | 87453157 |
| rs17339927 | rs4842602 | 1 | 0.555556 | 8.78E−13 | Chr12 | 87453479 |
| rs11104935 | rs4842602 | 1 | 0.555556 | 8.78E−13 | Chr12 | 87454209 |
| rs11104937 | rs4842602 | 1 | 0.555556 | 8.78E−13 | Chr12 | 87454857 |
| rs4842632 | rs4842602 | 1 | 0.555556 | 8.78E−13 | Chr12 | 87455663 |
| rs10506956 | rs4842602 | 1 | 0.555556 | 8.78E−13 | Chr12 | 87456166 |
| rs11104939 | rs4842602 | 1 | 0.555556 | 8.78E−13 | Chr12 | 87456495 |
| rs11104940 | rs4842602 | 1 | 0.555556 | 8.78E−13 | Chr12 | 87456855 |
| rs10858757 | rs4842602 | 1 | 0.555556 | 8.78E−13 | Chr12 | 87456935 |
| rs11104941 | rs4842602 | 1 | 0.555556 | 8.78E−13 | Chr12 | 87457472 |
| rs11104942 | rs4842602 | 1 | 0.588235 | 5.12E−13 | Chr12 | 87457600 |
| rs7135958 | rs1022034 | 1 | 1 | 9.73E−12 | Chr12 | 87457875 |
| rs7964695 | rs1022034 | 1 | 0.885387 | 6.11E−14 | Chr12 | 87459760 |
| rs1492348 | rs1022034 | 1 | 0.363958 | 4.29E−06 | Chr12 | 87460779 |
| rs3782174 | rs4842602 | 1 | 0.533981 | 6.78E−12 | Chr12 | 87463008 |
| rs3782175 | rs4842602 | 1 | 0.555556 | 1.09E−12 | Chr12 | 87463206 |
| rs3782176 | rs1022034 | 0.853281 | 0.676684 | 3.06E−09 | Chr12 | 87463264 |
| rs4842633 | rs4842602 | 1 | 0.555556 | 1.21E−12 | Chr12 | 87465142 |
| rs11104947 | rs1022034 | 1 | 0.26893 | 0.000083 | Chr12 | 87467111 |
| rs2407206 | rs3782181 | 1 | 1 | 1.32E−26 | Chr12 | 87467454 |
| rs11104948 | rs4842602 | 1 | 0.533981 | 6.11E−12 | Chr12 | 87467924 |
| rs1492351 | rs3782181 | 1 | 1 | 1.32E−26 | Chr12 | 87468140 |
| rs10858758 | rs3782181 | 1 | 1 | 1.32E−26 | Chr12 | 87468649 |
| rs1000788 | rs3782181 | 1 | 1 | 1.35E−25 | Chr12 | 87470908 |
| rs1466692 | rs1022034 | 0.84151 | 0.469626 | 2.90E−07 | Chr12 | 87470918 |
| rs1466691 | rs1022034 | 0.882899 | 0.707476 | 2.47E−11 | Chr12 | 87471002 |
| rs1492354 | rs1022034 | 0.724805 | 0.401036 | 1.42E−06 | Chr12 | 87472109 |
| rs12370494 | rs4842602 | 1 | 0.555556 | 8.78E−13 | Chr12 | 87472834 |
| rs7312974 | rs3782181 | 1 | 1 | 4.80E−26 | Chr12 | 87473003 |
| rs1703081 | rs1022034 | 0.724805 | 0.401036 | 1.42E−06 | Chr12 | 87475518 |
| rs1472899 | rs3782181 | 1 | 1 | 1.32E−26 | Chr12 | 87475616 |
| rs3782179 | rs3782181 | 1 | 1 | 1.32E−26 | Chr12 | 87477457 |
| rs3782180 | rs3782181 | 1 | 0.946346 | 8.21E−24 | Chr12 | 87477530 |
| rs3782181 | rs3782181 | 1 | 1 | 0 | Chr12 | 87477692 |
| rs4474514 | rs3782181 | 1 | 1 | 1.32E−26 | Chr12 | 87478090 |
| rs1907702 | rs3782181 | 1 | 1 | 4.65E−24 | Chr12 | 87479600 |
| rs11104952 | rs3782181 | 1 | 0.946237 | 8.42E−25 | Chr12 | 87480531 |
| rs1798011 | rs1022034 | 0.724805 | 0.401036 | 1.42E−06 | Chr12 | 87480756 |
| rs2046971 | rs3782181 | 1 | 1 | 1.32E−26 | Chr12 | 87483641 |
| rs1352947 | rs3782181 | 1 | 0.894598 | 2.58E−22 | Chr12 | 87484858 |
| rs10777129 | rs1022034 | 0.756098 | 0.498841 | 5.56E−08 | Chr12 | 87485844 |
| rs10777131 | rs1022034 | 0.706925 | 0.362779 | 0.000132 | Chr12 | 87486142 |
| rs12146799 | rs4842602 | 1 | 0.555556 | 9.78E−13 | Chr12 | 87486812 |
| rs10506957 | rs3782181 | 1 | 0.894598 | 2.58E−22 | Chr12 | 87491138 |
| rs869408 | rs3782181 | 1 | 0.894598 | 6.81E−22 | Chr12 | 87498886 |
| rs12368722 | rs4842602 | 0.906428 | 0.49911 | 2.45E−10 | Chr12 | 87502688 |
| rs1508595 | rs3782181 | 1 | 0.843478 | 1.07E−20 | Chr12 | 87510147 |
| rs17340767 | rs4842602 | 1 | 0.555556 | 8.78E−13 | Chr12 | 87513880 |
| rs12371735 | rs4842602 | 1 | 0.555556 | 8.78E−13 | Chr12 | 87514092 |
| rs931936 | rs995030 | 1 | 0.668966 | 8.45E−16 | Chr12 | 87518063 |
| rs11104966 | rs1022034 | 1 | 0.26893 | 0.000083 | Chr12 | 87527706 |
| rs2639099 | rs1022034 | 0.724805 | 0.401036 | 1.42E−06 | Chr12 | 87532495 |
| rs11104967 | rs4842602 | 1 | 0.555556 | 8.78E−13 | Chr12 | 87533006 |
| rs2639101 | rs3782181 | 1 | 0.843478 | 1.07E−20 | Chr12 | 87536983 |
| rs1492355 | rs3782181 | 1 | 0.843478 | 1.07E−20 | Chr12 | 87537273 |
| rs17425489 | rs4842602 | 1 | 0.357143 | 1.78E−08 | Chr12 | 87539269 |
| rs12367952 | rs4842602 | 1 | 0.555556 | 8.78E−13 | Chr12 | 87541280 |
| rs1703078 | rs3782181 | 1 | 0.383399 | 5.39E−09 | Chr12 | 87543014 |
| rs3861096 | rs3782181 | 1 | 0.273901 | 3.94E−07 | Chr12 | 87551973 |
| rs6538157 | rs3782181 | 1 | 0.273901 | 3.94E−07 | Chr12 | 87553955 |
| rs7295622 | rs3782181 | 1 | 0.273901 | 3.94E−07 | Chr12 | 87555020 |
| rs964881 | rs3782181 | 1 | 0.273901 | 3.94E−07 | Chr12 | 87557882 |
| rs4073022 | rs3782181 | 1 | 0.273901 | 3.94E−07 | Chr12 | 87561503 |
| rs1881227 | rs3782181 | 1 | 0.273901 | 3.94E−07 | Chr12 | 87562931 |
| rs7953625 | rs3782181 | 1 | 0.273901 | 3.94E−07 | Chr12 | 87569530 |
| rs2407522 | rs3782181 | 1 | 0.273901 | 3.94E−07 | Chr12 | 87586557 |
| rs7967010 | rs3782181 | 1 | 0.273901 | 3.94E−07 | Chr12 | 87590557 |
| rs10858774 | rs3782181 | 1 | 0.273901 | 3.94E−07 | Chr12 | 87600240 |
| rs1406091 | rs3782181 | 1 | 0.273901 | 3.94E−07 | Chr12 | 87602570 |
| rs10777134 | rs3782181 | 1 | 0.273901 | 3.94E−07 | Chr12 | 87615471 |
| rs10858776 | rs3782181 | 1 | 0.273901 | 3.94E−07 | Chr12 | 87622059 |
| rs10777136 | rs3782181 | 1 | 0.273901 | 3.94E−07 | Chr12 | 87622621 |
| rs10777137 | rs3782181 | 1 | 0.273901 | 3.94E−07 | Chr12 | 87633729 |
| rs10745490 | rs3782181 | 1 | 0.273901 | 3.94E−07 | Chr12 | 87640601 |
| rs7314684 | rs3782181 | 1 | 0.273901 | 3.94E−07 | Chr12 | 87643990 |
| rs11105028 | rs3782181 | 1 | 0.273901 | 3.94E−07 | Chr12 | 87644447 |
| rs1026654 | rs3782181 | 1 | 0.273901 | 3.94E−07 | Chr12 | 87652131 |
| rs12426031 | rs3782181 | 1 | 0.273901 | 3.94E−07 | Chr12 | 87660825 |
| rs7306543 | rs3782181 | 1 | 0.273901 | 3.94E−07 | Chr12 | 87670667 |
| rs12816527 | rs12821256 | 0.930796 | 0.810304 | 1.49E−17 | Chr12 | 87688214 |
| rs1398305 | rs1022034 | 1 | 0.243499 | 0.001439 | Chr12 | 87753163 |
| rs12822439 | rs12821256 | 0.931215 | 0.863449 | 8.34E−18 | Chr12 | 87759779 |
| rs10506960 | rs1022034 | 1 | 0.329073 | 0.000081 | Chr12 | 87779978 |
| rs12821256 | rs12821256 | 1 | 1 | 0 | Chr12 | 87852466 |
| rs399962 | rs1022034 | 0.454689 | 0.202388 | 0.000422 | Chr12 | 87859455 |
| rs11105124 | rs1022034 | 1 | 0.26893 | 0.000083 | Chr12 | 87900040 |
| rs4842644 | rs12821256 | 0.7411 | 0.269812 | 4.20E−06 | Chr12 | 88020409 |
| rs17430515 | rs12821256 | 0.510775 | 0.228896 | 5.22E−06 | Chr12 | 88052931 |
| rs1996640 | rs12821256 | 0.510775 | 0.228896 | 5.22E−06 | Chr12 | 88059992 |
| rs1949538 | rs12821256 | 0.510775 | 0.228896 | 5.22E−06 | Chr12 | 88097863 |
| rs12811742 | rs12821256 | 0.510775 | 0.228896 | 5.22E−06 | Chr12 | 88124550 |
| rs4842646 | rs12821256 | 0.510775 | 0.228896 | 5.22E−06 | Chr12 | 88126801 |
| rs4900106 | rs2402130 | 0.500824 | 0.206991 | 0.00002 | Chr14 | 91808898 |
| rs4904862 | rs8016079 | 1 | 0.310345 | 8.61E−06 | Chr14 | 91820654 |
| rs4900107 | rs8016079 | 1 | 0.348659 | 4.98E−10 | Chr14 | 91823565 |
| rs8016079 | rs8016079 | 1 | 1 | 0 | Chr14 | 91828198 |
| rs12434284 | rs2402130 | 0.848676 | 0.25758 | 3.42E−06 | Chr14 | 91829264 |
| rs8017054 | rs4904868 | 1 | 1 | 6.02E−34 | Chr14 | 91830169 |
| rs1951351 | rs4904864 | 1 | 1 | 3.20E−29 | Chr14 | 91832947 |
| rs4900109 | rs4904868 | 1 | 0.659179 | 2.62E−22 | Chr14 | 91833144 |
| rs4904864 | rs4904864 | 1 | 1 | 0 | Chr14 | 91834272 |
| rs1885193 | rs4904864 | 1 | 1 | 1.15E−29 | Chr14 | 91835107 |
| rs12892379 | rs4904864 | 1 | 1 | 1.48E−29 | Chr14 | 91836910 |
| rs1957283 | rs2402130 | 1 | 0.454545 | 1.44E−10 | Chr14 | 91837433 |
| rs4904866 | rs4904868 | 1 | 0.668675 | 8.53E−23 | Chr14 | 91838256 |
| rs7145248 | rs2402130 | 1 | 0.454545 | 1.44E−10 | Chr14 | 91839145 |
| rs12434570 | rs2402130 | 1 | 0.454545 | 1.44E−10 | Chr14 | 91840166 |
| rs8021744 | rs2402130 | 1 | 0.454545 | 1.44E−10 | Chr14 | 91843048 |
| rs12896399 | rs4904868 | 1 | 0.668675 | 8.53E−23 | Chr14 | 91843416 |
| rs746588 | rs2402130 | 1 | 0.393258 | 2.29E−08 | Chr14 | 91845133 |
| rs11622569 | rs4904864 | 1 | 1 | 3.20E−29 | Chr14 | 91845155 |
| rs746586 | rs4904868 | 1 | 0.665569 | 3.12E−22 | Chr14 | 91845720 |
| rs1075830 | rs4904868 | 1 | 1 | 4.16E−34 | Chr14 | 91845915 |
| rs8018017 | rs8016079 | 0.678365 | 0.25656 | 0.00007 | Chr14 | 91846171 |
| rs941799 | rs4904868 | 1 | 0.659179 | 2.62E−22 | Chr14 | 91846578 |
| rs1885194 | rs4904868 | 1 | 0.668675 | 8.53E−23 | Chr14 | 91847215 |
| rs10484035 | rs4904864 | 1 | 1 | 1.15E−29 | Chr14 | 91848310 |
| rs17184180 | rs4904868 | 1 | 0.665569 | 1.24E−22 | Chr14 | 91850140 |
| rs4904868 | rs4904868 | 1 | 1 | 0 | Chr14 | 91850754 |
| rs4904870 | rs4904868 | 1 | 1 | 6.02E−34 | Chr14 | 91856761 |
| rs4900112 | rs4904864 | 1 | 1 | 1.91E−29 | Chr14 | 91857042 |
| rs7149739 | rs4904864 | 1 | 1 | 1.15E−29 | Chr14 | 91864509 |
| rs4900114 | rs4904868 | 0.960919 | 0.922925 | 1.16E−28 | Chr14 | 91865488 |
| rs11160056 | rs2402130 | 1 | 0.454545 | 1.44E−10 | Chr14 | 91866623 |
| rs11160057 | rs2402130 | 1 | 0.454545 | 1.44E−10 | Chr14 | 91866998 |
| rs8014907 | rs2402130 | 1 | 1 | 3.02E−25 | Chr14 | 91869757 |
| rs2402130 | rs2402130 | 1 | 1 | 0 | Chr14 | 91870956 |
| rs4600402 | rs2402130 | 1 | 0.9375 | 4.43E−22 | Chr14 | 91873669 |
| rs12434625 | rs2402130 | 1 | 0.421687 | 1.18E−08 | Chr14 | 91875229 |
| rs12434665 | rs2402130 | 1 | 1 | 3.02E−25 | Chr14 | 91875238 |
| rs12435365 | rs2402130 | 1 | 0.377358 | 1.19E−08 | Chr14 | 91875964 |
| rs12432529 | rs2402130 | 1 | 1 | 2.51E−25 | Chr14 | 91876082 |
| rs2896206 | rs2402130 | 0.59592 | 0.253658 | 3.17E−06 | Chr14 | 91887414 |
| rs10873421 | rs2402130 | 0.612984 | 0.294446 | 2.26E−06 | Chr14 | 91888484 |
| rs10782477 | rs2402130 | 0.609938 | 0.276391 | 1.72E−06 | Chr14 | 91888854 |
| rs11623214 | rs2402130 | 0.89589 | 0.426428 | 2.11E−09 | Chr14 | 91891269 |
| rs4243693 | rs2402130 | 1 | 0.309735 | 1.60E−07 | Chr14 | 91891819 |
| rs4904876 | rs2402130 | 1 | 0.327103 | 1.07E−07 | Chr14 | 91892443 |
| rs7159028 | rs2402130 | 1 | 0.309735 | 1.81E−07 | Chr14 | 91892520 |
| rs7142084 | rs2402130 | 1 | 0.309735 | 1.60E−07 | Chr14 | 91892784 |
| rs7142440 | rs2402130 | 1 | 0.309735 | 1.60E−07 | Chr14 | 91892908 |
| rs7142805 | rs2402130 | 1 | 0.327103 | 1.07E−07 | Chr14 | 91893149 |
| rs8182049 | rs7183877 | 1 | 0.322034 | 0.000378 | Chr15 | 25332700 |
| rs1580790 | rs1498519 | 0.534836 | 0.216121 | 3.87E−06 | Chr15 | 25620761 |
| rs4778174 | rs1498519 | 0.69481 | 0.270984 | 2.36E−07 | Chr15 | 25643161 |
| rs12916614 | rs1498519 | 0.846479 | 0.377833 | 6.89E−11 | Chr15 | 25652229 |
| rs4622464 | rs1498519 | 0.835811 | 0.341352 | 7.12E−10 | Chr15 | 25653220 |
| rs1498513 | rs1498519 | 0.847241 | 0.384124 | 5.87E−11 | Chr15 | 25653607 |
| rs1498512 | rs1498519 | 0.826308 | 0.30617 | 1.90E−09 | Chr15 | 25653631 |
| rs1498511 | rs1498519 | 0.833543 | 0.333088 | 7.12E−10 | Chr15 | 25653701 |
| rs1353737 | rs1498519 | 0.923235 | 0.237997 | 2.35E−08 | Chr15 | 25655510 |
| rs12440600 | rs1498519 | 0.83665 | 0.3384 | 5.39E−10 | Chr15 | 25655741 |
| rs11635999 | rs1498519 | 0.84552 | 0.369853 | 8.17E−11 | Chr15 | 25656485 |
| rs12907793 | rs1498519 | 0.84552 | 0.369853 | 8.17E−11 | Chr15 | 25656548 |
| rs17565757 | rs1498519 | 0.902796 | 0.425788 | 1.72E−13 | Chr15 | 25657780 |
| rs11633586 | rs1498519 | 0.825869 | 0.629128 | 7.13E−15 | Chr15 | 25657967 |
| rs7175224 | rs1498519 | 0.884971 | 0.360049 | 9.72E−11 | Chr15 | 25658967 |
| rs12437621 | rs1498519 | 0.835811 | 0.341352 | 7.12E−10 | Chr15 | 25659209 |
| rs4322616 | rs1498519 | 0.796436 | 0.342611 | 5.59E−10 | Chr15 | 25661583 |
| rs4402510 | rs1498519 | 0.852736 | 0.406076 | 7.08E−12 | Chr15 | 25661673 |
| rs4402511 | rs1498519 | 0.847955 | 0.645947 | 2.10E−18 | Chr15 | 25661691 |
| rs4423373 | rs1498519 | 0.844935 | 0.372959 | 5.32E−11 | Chr15 | 25661935 |
| rs7164438 | rs1498519 | 0.829413 | 0.485644 | 9.03E−14 | Chr15 | 25667051 |
| rs11633934 | rs1498519 | 0.832271 | 0.50584 | 2.43E−14 | Chr15 | 25668326 |
| rs17650960 | rs1498519 | 1 | 1 | 6.30E−36 | Chr15 | 25673037 |
| rs11074304 | rs1498519 | 1 | 0.427044 | 2.53E−16 | Chr15 | 25674612 |
| rs7164752 | rs1498519 | 1 | 0.432289 | 2.23E−16 | Chr15 | 25675654 |
| rs1603784 | rs1498519 | 1 | 0.229947 | 4.78E−10 | Chr15 | 25677384 |
| rs989869 | rs1498519 | 1 | 0.427044 | 2.53E−16 | Chr15 | 25679901 |
| rs11633701 | rs1498519 | 1 | 1 | 6.30E−36 | Chr15 | 25681071 |
| rs11637518 | rs1498519 | 1 | 1 | 6.30E−36 | Chr15 | 25683151 |
| rs1498519 | rs1498519 | 1 | 1 | 0 | Chr15 | 25685246 |
| rs3947367 | rs1498519 | 1 | 0.25879 | 5.18E−11 | Chr15 | 25687699 |
| rs17565953 | rs6497238 | 0.957299 | 0.645291 | 4.04E−20 | Chr15 | 25692250 |
| rs17651026 | rs1584407 | 0.872718 | 0.276959 | 1.44E−06 | Chr15 | 25692912 |
| rs8023340 | rs6497238 | 0.952111 | 0.50467 | 2.58E−16 | Chr15 | 25693983 |
| rs11636948 | rs6497238 | 0.951908 | 0.500534 | 3.94E−16 | Chr15 | 25694490 |
| rs2279728 | rs6497238 | 0.950727 | 0.499708 | 7.86E−16 | Chr15 | 25695098 |
| rs11631195 | rs6497238 | 0.961224 | 0.798515 | 9.95E−25 | Chr15 | 25695483 |
| rs884197 | rs6497238 | 1 | 0.503722 | 1.38E−18 | Chr15 | 25700695 |
| rs8036798 | rs6497238 | 0.96126 | 0.800406 | 6.53E−25 | Chr15 | 25703085 |
| rs1498509 | rs6497238 | 1 | 0.503722 | 1.38E−18 | Chr15 | 25703595 |
| rs11631057 | rs6497238 | 0.899082 | 0.736715 | 2.23E−17 | Chr15 | 25703753 |
| rs7171246 | rs6497238 | 0.961224 | 0.798515 | 9.95E−25 | Chr15 | 25708504 |
| rs11858694 | rs6497238 | 0.96113 | 0.800189 | 2.64E−24 | Chr15 | 25709487 |
| rs12591640 | rs6497238 | 0.957162 | 0.623626 | 9.28E−20 | Chr15 | 25712846 |
| rs3930739 | rs6497238 | 0.902235 | 0.438323 | 7.15E−14 | Chr15 | 25713937 |
| rs17674017 | rs6497238 | 0.875152 | 0.557957 | 7.49E−17 | Chr15 | 25714352 |
| rs4238492 | rs6497238 | 0.904785 | 0.455746 | 1.20E−14 | Chr15 | 25715499 |
| rs4238493 | rs6497238 | 0.903822 | 0.466533 | 4.57E−14 | Chr15 | 25715522 |
| rs4238494 | rs6497238 | 0.889489 | 0.441114 | 6.81E−12 | Chr15 | 25715534 |
| rs6497233 | rs6497238 | 1 | 1 | 1.15E−35 | Chr15 | 25716985 |
| rs12911001 | rs6497238 | 0.95803 | 0.737878 | 1.44E−21 | Chr15 | 25718030 |
| rs11074306 | rs6497238 | 1 | 0.397993 | 2.05E−15 | Chr15 | 25718137 |
| rs8036440 | rs6497238 | 1 | 1 | 3.63E−32 | Chr15 | 25719110 |
| rs2311472 | rs6497238 | 1 | 0.78022 | 7.13E−27 | Chr15 | 25721364 |
| rs12914516 | rs6497238 | 1 | 0.78022 | 7.13E−27 | Chr15 | 25721787 |
| rs4778181 | rs6497238 | 1 | 0.857398 | 3.25E−28 | Chr15 | 25721908 |
| rs6497234 | rs6497238 | 1 | 1 | 1.15E−35 | Chr15 | 25722330 |
| rs6497235 | rs6497238 | 1 | 0.78022 | 7.13E−27 | Chr15 | 25722899 |
| rs4778182 | rs6497238 | 1 | 1 | 1.15E−35 | Chr15 | 25723106 |
| rs2055291 | rs2594935 | 0.756575 | 0.228621 | 0.000023 | Chr15 | 25724179 |
| rs2055290 | rs6497238 | 1 | 0.760706 | 3.09E−21 | Chr15 | 25724285 |
| rs6497236 | rs6497238 | 1 | 1 | 9.53E−35 | Chr15 | 25725177 |
| rs6497237 | rs6497238 | 1 | 0.96008 | 1.76E−31 | Chr15 | 25725219 |
| rs12905726 | rs6497238 | 1 | 1 | 9.53E−35 | Chr15 | 25725669 |
| rs17651145 | rs6497238 | 1 | 0.964222 | 1.37E−33 | Chr15 | 25725894 |
| rs8037607 | rs6497238 | 1 | 1 | 3.31E−35 | Chr15 | 25727078 |
| rs8036718 | rs6497238 | 1 | 1 | 3.31E−35 | Chr15 | 25727152 |
| rs6497238 | rs6497238 | 1 | 1 | 0 | Chr15 | 25727373 |
| rs924315 | rs6497238 | 1 | 1 | 3.31E−35 | Chr15 | 25727739 |
| rs924314 | rs6497238 | 1 | 0.629101 | 4.05E−22 | Chr15 | 25727941 |
| rs1603787 | rs6497238 | 1 | 1 | 9.53E−35 | Chr15 | 25729127 |
| rs7161969 | rs6497238 | 0.961051 | 0.858065 | 6.10E−27 | Chr15 | 25730107 |
| rs1874840 | rs6497238 | 0.845733 | 0.635495 | 4.30E−18 | Chr15 | 25731189 |
| rs924313 | rs6497238 | 0.737637 | 0.524642 | 7.89E−15 | Chr15 | 25731445 |
| rs924312 | rs6497238 | 0.846412 | 0.642935 | 8.48E−19 | Chr15 | 25731458 |
| rs1498521 | rs6497238 | 0.846412 | 0.642935 | 8.48E−19 | Chr15 | 25732503 |
| rs2201874 | rs6497238 | 0.842748 | 0.639643 | 5.59E−18 | Chr15 | 25733786 |
| rs8042881 | rs6497238 | 0.842748 | 0.639643 | 5.59E−18 | Chr15 | 25734224 |
| rs4778185 | rs6497238 | 1 | 0.654676 | 5.91E−23 | Chr15 | 25738936 |
| rs2311469 | rs6497238 | 1 | 0.636364 | 1.69E−22 | Chr15 | 25742663 |
| rs4580105 | rs6497238 | 1 | 0.632768 | 2.62E−22 | Chr15 | 25744036 |
| rs17747086 | rs6497238 | 1 | 0.632768 | 2.62E−22 | Chr15 | 25744335 |
| rs9806334 | rs6497238 | 1 | 0.632768 | 2.62E−22 | Chr15 | 25745162 |
| rs4778188 | rs6497238 | 1 | 0.860627 | 7.68E−28 | Chr15 | 25745282 |
| rs7163017 | rs6497238 | 1 | 0.632768 | 2.62E−22 | Chr15 | 25745752 |
| rs4778189 | rs6497238 | 1 | 0.636364 | 1.69E−22 | Chr15 | 25745823 |
| rs8041183 | rs6497238 | 1 | 0.629101 | 8.86E−22 | Chr15 | 25746490 |
| rs9806561 | rs6497238 | 1 | 0.651163 | 9.16E−23 | Chr15 | 25746649 |
| rs9806736 | rs6497238 | 1 | 0.632768 | 2.62E−22 | Chr15 | 25747076 |
| rs8037478 | rs6497238 | 1 | 0.663609 | 8.29E−22 | Chr15 | 25750424 |
| rs4778190 | rs6497238 | 1 | 0.857398 | 4.77E−28 | Chr15 | 25750755 |
| rs4778191 | rs6497238 | 1 | 0.632768 | 2.62E−22 | Chr15 | 25750903 |
| rs1391623 | rs6497238 | 1 | 0.860627 | 2.43E−28 | Chr15 | 25751557 |
| rs10162623 | rs6497238 | 1 | 0.632768 | 2.62E−22 | Chr15 | 25752031 |
| rs4778192 | rs6497238 | 1 | 0.636364 | 1.69E−22 | Chr15 | 25753656 |
| rs4778193 | rs6497238 | 1 | 0.860627 | 1.65E−28 | Chr15 | 25753761 |
| rs8035334 | rs6497238 | 1 | 0.57162 | 1.63E−20 | Chr15 | 25753962 |
| rs11858340 | rs6497238 | 0.839697 | 0.566927 | 9.20E−17 | Chr15 | 25754179 |
| rs11852452 | rs6497238 | 0.839697 | 0.566927 | 9.20E−17 | Chr15 | 25754287 |
| rs17566358 | rs6497238 | 0.839697 | 0.566927 | 9.20E−17 | Chr15 | 25754455 |
| rs2311470 | rs6497238 | 1 | 0.57162 | 3.38E−20 | Chr15 | 25755617 |
| rs7164946 | rs6497238 | 0.839697 | 0.566927 | 9.20E−17 | Chr15 | 25755765 |
| rs17651351 | rs1584407 | 0.875661 | 0.29073 | 9.33E−07 | Chr15 | 25755846 |
| rs8023273 | rs6497238 | 1 | 0.963768 | 2.12E−33 | Chr15 | 25756424 |
| rs1018104 | rs6497238 | 0.730946 | 0.421596 | 1.79E−11 | Chr15 | 25756802 |
| rs1018105 | rs6497238 | 0.807917 | 0.460822 | 3.20E−13 | Chr15 | 25757018 |
| rs1391625 | rs6497238 | 0.766996 | 0.433102 | 2.05E−12 | Chr15 | 25758733 |
| rs4778194 | rs1584407 | 0.945105 | 0.850429 | 7.07E−21 | Chr15 | 25758795 |
| rs4778196 | rs1584407 | 0.936559 | 0.51257 | 3.43E−13 | Chr15 | 25759991 |
| rs8035720 | rs6497238 | 0.766996 | 0.433102 | 2.05E−12 | Chr15 | 25760176 |
| rs2133248 | rs1584407 | 1 | 0.948454 | 2.06E−25 | Chr15 | 25760332 |
| rs12591662 | rs1584407 | 1 | 0.941296 | 1.50E−22 | Chr15 | 25762821 |
| rs12592307 | rs1584407 | 1 | 0.946237 | 8.42E−25 | Chr15 | 25763768 |
| rs4778197 | rs1584407 | 0.94552 | 0.734364 | 1.51E−18 | Chr15 | 25765296 |
| rs8029469 | rs6497238 | 0.640886 | 0.343512 | 2.80E−10 | Chr15 | 25766195 |
| rs4778198 | rs6497238 | 0.767258 | 0.42658 | 3.87E−12 | Chr15 | 25766877 |
| rs924318 | rs6497238 | 0.698317 | 0.400517 | 2.22E−11 | Chr15 | 25767029 |
| rs1874841 | rs1584407 | 0.943867 | 0.668163 | 7.26E−17 | Chr15 | 25769916 |
| rs7163379 | rs1584407 | 0.939228 | 0.514587 | 1.05E−13 | Chr15 | 25774171 |
| rs7172625 | rs1584407 | 0.937987 | 0.484796 | 5.47E−13 | Chr15 | 25774388 |
| rs768547 | rs1584407 | 1 | 0.948454 | 1.66E−25 | Chr15 | 25774538 |
| rs768546 | rs1584407 | 0.94552 | 0.734364 | 1.51E−18 | Chr15 | 25774732 |
| rs768545 | rs6497238 | 0.694954 | 0.319107 | 3.38E−08 | Chr15 | 25774803 |
| rs4778199 | rs1584407 | 0.939228 | 0.514587 | 1.05E−13 | Chr15 | 25780182 |
| rs4778200 | rs1584407 | 0.94552 | 0.734364 | 1.51E−18 | Chr15 | 25781616 |
| rs1973448 | rs6497238 | 0.728422 | 0.407053 | 1.11E−11 | Chr15 | 25781906 |
| rs7177529 | rs1584407 | 0.939228 | 0.514587 | 1.05E−13 | Chr15 | 25785341 |
| rs4238495 | rs6497238 | 0.728422 | 0.407053 | 1.11E−11 | Chr15 | 25787869 |
| rs4778201 | rs6497238 | 0.728422 | 0.407053 | 1.11E−11 | Chr15 | 25787884 |
| rs3903042 | rs1498519 | 0.704945 | 0.201218 | 1.95E−06 | Chr15 | 25788679 |
| rs12594397 | rs1498519 | 0.705766 | 0.205599 | 1.87E−06 | Chr15 | 25789768 |
| rs7175046 | rs6497238 | 0.734579 | 0.433259 | 1.74E−11 | Chr15 | 25789870 |
| rs12440997 | rs1584407 | 1 | 0.656489 | 2.28E−19 | Chr15 | 25792039 |
| rs6497249 | rs6497238 | 0.724192 | 0.404736 | 6.99E−11 | Chr15 | 25794308 |
| rs7161804 | rs1584407 | 1 | 0.58042 | 8.56E−18 | Chr15 | 25794860 |
| rs4598864 | rs1584407 | 1 | 0.578847 | 1.91E−17 | Chr15 | 25795454 |
| rs7403465 | rs6497238 | 0.769594 | 0.434859 | 1.56E−12 | Chr15 | 25798717 |
| rs2594885 | rs1584407 | 1 | 0.583333 | 6.82E−18 | Chr15 | 25800968 |
| rs2703969 | rs1584407 | 1 | 0.583333 | 6.82E−18 | Chr15 | 25805228 |
| rs4778129 | rs1584407 | 1 | 0.713115 | 1.03E−18 | Chr15 | 25806025 |
| rs1823273 | rs1584407 | 1 | 0.583333 | 6.82E−18 | Chr15 | 25806440 |
| rs1375166 | rs1584407 | 1 | 1 | 6.03E−28 | Chr15 | 25806969 |
| rs1839146 | rs6497238 | 0.769594 | 0.434859 | 1.56E−12 | Chr15 | 25807486 |
| rs4589506 | rs1584407 | 1 | 0.632353 | 6.53E−19 | Chr15 | 25807809 |
| rs2442124 | rs1584407 | 1 | 0.583333 | 6.82E−18 | Chr15 | 25809801 |
| rs2594890 | rs6497238 | 0.769594 | 0.434859 | 1.56E−12 | Chr15 | 25810905 |
| rs2594891 | rs1584407 | 0.882164 | 0.492106 | 7.32E−13 | Chr15 | 25811118 |
| rs895832 | rs1584407 | 1 | 0.583333 | 6.82E−18 | Chr15 | 25813455 |
| rs2594893 | rs1584407 | 1 | 0.583333 | 6.82E−18 | Chr15 | 25814248 |
| rs2594895 | rs1584407 | 1 | 0.583333 | 6.82E−18 | Chr15 | 25814899 |
| rs1597198 | rs1584407 | 1 | 0.62963 | 8.18E−19 | Chr15 | 25816203 |
| rs895833 | rs1584407 | 1 | 0.62963 | 8.18E−19 | Chr15 | 25817967 |
| rs2871886 | rs1584407 | 1 | 0.62406 | 4.58E−18 | Chr15 | 25818619 |
| rs2594897 | rs6497238 | 0.766418 | 0.425364 | 1.40E−11 | Chr15 | 25818645 |
| rs2594898 | rs6497238 | 0.766418 | 0.425364 | 1.40E−11 | Chr15 | 25818665 |
| rs11854269 | rs1584407 | 1 | 0.62963 | 8.18E−19 | Chr15 | 25820388 |
| rs2703978 | rs6497238 | 0.763831 | 0.416629 | 3.51E−11 | Chr15 | 25820801 |
| rs2594901 | rs6497238 | 0.766418 | 0.425364 | 1.40E−11 | Chr15 | 25821055 |
| rs17674604 | rs1584407 | 1 | 0.626866 | 1.03E−18 | Chr15 | 25821537 |
| rs4778130 | rs1584407 | 1 | 0.62963 | 8.18E−19 | Chr15 | 25821832 |
| rs8028640 | rs6497238 | 0.622779 | 0.322683 | 7.14E−09 | Chr15 | 25823292 |
| rs2594907 | rs6497238 | 0.638478 | 0.340935 | 5.35E−10 | Chr15 | 25823517 |
| rs2703983 | rs6497238 | 0.638478 | 0.340935 | 5.35E−10 | Chr15 | 25824156 |
| rs2594908 | rs6497238 | 0.638478 | 0.340935 | 5.35E−10 | Chr15 | 25824237 |
| rs2594909 | rs6497238 | 0.667848 | 0.350383 | 5.38E−09 | Chr15 | 25824657 |
| rs2594911 | rs6497238 | 0.638478 | 0.340935 | 5.35E−10 | Chr15 | 25826193 |
| rs2703921 | rs6497238 | 0.635309 | 0.350279 | 4.92E−10 | Chr15 | 25826268 |
| rs2703922 | rs6497238 | 0.638478 | 0.340935 | 5.35E−10 | Chr15 | 25826923 |
| rs2703923 | rs6497238 | 0.612372 | 0.321502 | 1.50E−08 | Chr15 | 25827188 |
| rs12593437 | rs6497238 | 0.644131 | 0.3594 | 1.76E−10 | Chr15 | 25828351 |
| rs13329141 | rs6497238 | 0.633463 | 0.3356 | 1.95E−09 | Chr15 | 25828788 |
| rs13329497 | rs6497238 | 0.630506 | 0.335109 | 1.08E−09 | Chr15 | 25828824 |
| rs7497038 | rs6497238 | 0.508917 | 0.235848 | 2.75E−06 | Chr15 | 25829515 |
| rs1584407 | rs1584407 | 1 | 1 | 0 | Chr15 | 25830854 |
| rs2594887 | rs6497238 | 0.633463 | 0.3356 | 1.95E−09 | Chr15 | 25832490 |
| rs12439410 | rs1584407 | 1 | 1 | 3.82E−27 | Chr15 | 25834159 |
| rs11074310 | rs6497238 | 0.617362 | 0.308156 | 1.48E−08 | Chr15 | 25835077 |
| rs2122006 | rs6497238 | 0.645388 | 0.358637 | 1.24E−10 | Chr15 | 25839474 |
| rs2594919 | rs6497238 | 0.646303 | 0.361827 | 9.15E−11 | Chr15 | 25840354 |
| rs1448487 | rs6497238 | 0.646303 | 0.361827 | 9.15E−11 | Chr15 | 25843051 |
| rs1597199 | rs1584407 | 1 | 0.95 | 4.16E−26 | Chr15 | 25843713 |
| rs1597201 | rs1584407 | 1 | 0.95 | 4.16E−26 | Chr15 | 25844146 |
| rs2594925 | rs6497238 | 0.646303 | 0.361827 | 9.15E−11 | Chr15 | 25844289 |
| rs2594926 | rs6497238 | 0.645388 | 0.358637 | 1.24E−10 | Chr15 | 25844333 |
| rs3751651 | rs1584407 | 1 | 0.95 | 4.16E−26 | Chr15 | 25844723 |
| rs12440216 | rs1584407 | 1 | 0.95 | 2.02E−25 | Chr15 | 25845265 |
| rs1597204 | rs1584407 | 1 | 0.95 | 4.16E−26 | Chr15 | 25848396 |
| rs11074312 | rs6497238 | 0.656667 | 0.401057 | 8.20E−12 | Chr15 | 25849494 |
| rs17674689 | rs1584407 | 1 | 0.95 | 4.16E−26 | Chr15 | 25849870 |
| rs2703963 | rs6497238 | 0.644131 | 0.3594 | 1.76E−10 | Chr15 | 25850423 |
| rs4238496 | rs1584407 | 0.947368 | 0.852631 | 1.51E−21 | Chr15 | 25851008 |
| rs2703960 | rs6497238 | 0.601621 | 0.323689 | 1.91E−09 | Chr15 | 25851467 |
| rs977589 | rs2594935 | 0.923642 | 0.260807 | 4.67E−09 | Chr15 | 25853198 |
| rs2703956 | rs2594935 | 0.944917 | 0.599768 | 1.23E−16 | Chr15 | 25853495 |
| rs2703955 | rs2594935 | 1 | 0.857256 | 2.98E−23 | Chr15 | 25853697 |
| rs2594931 | rs2594935 | 0.9509 | 0.904211 | 1.57E−24 | Chr15 | 25853732 |
| rs2703954 | rs2703952 | 1 | 0.91453 | 3.63E−17 | Chr15 | 25853795 |
| rs2703952 | rs2703952 | 1 | 1 | 0 | Chr15 | 25855576 |
| rs12437597 | rs728405 | 1 | 0.263158 | 1.45E−06 | Chr15 | 25855901 |
| rs2594933 | rs2594935 | 1 | 1 | 4.08E−29 | Chr15 | 25855959 |
| rs2594934 | rs2594935 | 1 | 1 | 4.08E−29 | Chr15 | 25856310 |
| rs2311843 | rs2594935 | 1 | 0.439252 | 1.02E−11 | Chr15 | 25856949 |
| rs1375169 | rs2703952 | 1 | 1 | 1.16E−19 | Chr15 | 25857720 |
| rs1448490 | rs2594935 | 1 | 0.439252 | 1.02E−11 | Chr15 | 25858122 |
| rs2703950 | rs2703952 | 1 | 1 | 1.16E−19 | Chr15 | 25858467 |
| rs2703949 | rs2703952 | 1 | 1 | 1.16E−19 | Chr15 | 25858520 |
| rs2594935 | rs2594935 | 1 | 1 | 0 | Chr15 | 25858633 |
| rs1562591 | rs2703952 | 1 | 1 | 1.16E−19 | Chr15 | 25859068 |
| rs1562592 | rs2703952 | 1 | 1 | 1.16E−19 | Chr15 | 25859610 |
| rs2594937 | rs2594935 | 1 | 1 | 5.22E−29 | Chr15 | 25860055 |
| rs8034072 | rs2594935 | 1 | 0.439252 | 1.02E−11 | Chr15 | 25860253 |
| rs2594938 | rs2594935 | 1 | 0.723077 | 4.40E−19 | Chr15 | 25860580 |
| rs1545397 | rs728405 | 1 | 0.263158 | 1.45E−06 | Chr15 | 25861367 |
| rs7169225 | rs1448488 | 0.900471 | 0.77251 | 3.79E−20 | Chr15 | 25862599 |
| rs7170451 | rs1448488 | 0.892919 | 0.754692 | 9.92E−18 | Chr15 | 25865819 |
| rs4778210 | rs728405 | 1 | 0.263158 | 1.45E−06 | Chr15 | 25868077 |
| rs7173419 | rs728405 | 1 | 1 | 2.51E−25 | Chr15 | 25870416 |
| rs17566952 | rs2594935 | 1 | 0.223962 | 1.56E−06 | Chr15 | 25870480 |
| rs921221 | rs728405 | 1 | 1 | 2.51E−25 | Chr15 | 25872162 |
| rs12915041 | rs728405 | 1 | 1 | 2.51E−25 | Chr15 | 25872713 |
| rs4778211 | rs4778220 | 1 | 0.78972 | 3.24E−16 | Chr15 | 25872900 |
| rs728405 | rs728405 | 1 | 1 | 0 | Chr15 | 25873448 |
| rs2017966 | rs728405 | 1 | 1 | 3.63E−25 | Chr15 | 25873458 |
| rs728404 | rs728405 | 1 | 0.309735 | 1.60E−07 | Chr15 | 25873637 |
| rs7170989 | rs728405 | 1 | 1 | 2.51E−25 | Chr15 | 25874003 |
| rs9920172 | rs1448488 | 0.90442 | 0.817976 | 5.23E−22 | Chr15 | 25874249 |
| rs7176632 | rs4778220 | 1 | 0.858491 | 6.38E−18 | Chr15 | 25874596 |
| rs17567007 | rs16950979 | 0.649123 | 0.205278 | 0.002766 | Chr15 | 25875134 |
| rs11638265 | rs1448488 | 0.949189 | 0.85929 | 1.87E−22 | Chr15 | 25876168 |
| rs4778214 | rs2594935 | 0.892005 | 0.31963 | 8.13E−08 | Chr15 | 25879159 |
| rs11074314 | rs4778220 | 1 | 1 | 3.73E−22 | Chr15 | 25881612 |
| rs7165923 | rs2594935 | 0.892005 | 0.31963 | 8.13E−08 | Chr15 | 25882029 |
| rs4778218 | rs4778220 | 1 | 1 | 2.47E−21 | Chr15 | 25885353 |
| rs1800411 | rs1448488 | 1 | 1 | 1.15E−29 | Chr15 | 25885516 |
| rs4778219 | rs1448488 | 1 | 0.326316 | 5.43E−09 | Chr15 | 25887445 |
| rs11857036 | rs1448488 | 1 | 0.347604 | 1.16E−09 | Chr15 | 25887743 |
| rs1448488 | rs1448488 | 1 | 1 | 0 | Chr15 | 25890452 |
| rs11636005 | rs1448488 | 1 | 1 | 1.15E−29 | Chr15 | 25894342 |
| rs11634923 | rs1448488 | 1 | 1 | 1.15E−29 | Chr15 | 25894631 |
| rs4778220 | rs4778220 | 1 | 1 | 0 | Chr15 | 25894733 |
| rs7182323 | rs1448488 | 1 | 1 | 1.15E−29 | Chr15 | 25894924 |
| rs11631735 | rs1448488 | 1 | 1 | 1.15E−29 | Chr15 | 25896375 |
| rs12442009 | rs1448488 | 1 | 0.347604 | 1.16E−09 | Chr15 | 25897168 |
| rs12914687 | rs1448488 | 1 | 1 | 1.15E−29 | Chr15 | 25900136 |
| rs12903382 | rs1448488 | 1 | 1 | 3.00E−28 | Chr15 | 25900544 |
| rs4778135 | rs1448488 | 1 | 0.347604 | 1.56E−09 | Chr15 | 25900858 |
| rs12910433 | rs1448488 | 1 | 0.953917 | 8.86E−28 | Chr15 | 25902239 |
| rs11074318 | rs4778220 | 1 | 1 | 2.14E−21 | Chr15 | 25903005 |
| rs7163930 | rs4778220 | 1 | 1 | 2.79E−22 | Chr15 | 25903290 |
| rs1900758 | rs1448488 | 1 | 0.953917 | 8.86E−28 | Chr15 | 25903692 |
| rs1800410 | rs1448488 | 1 | 0.347604 | 1.42E−09 | Chr15 | 25903779 |
| rs4778221 | rs1448488 | 1 | 0.347604 | 1.16E−09 | Chr15 | 25904874 |
| rs1037208 | rs4778220 | 1 | 0.932127 | 1.30E−20 | Chr15 | 25904952 |
| rs10852218 | rs4778220 | 1 | 0.932127 | 1.30E−20 | Chr15 | 25905388 |
| rs730502 | rs1448488 | 1 | 0.279279 | 5.42E−08 | Chr15 | 25908005 |
| rs1800404 | rs2871875 | 0.842739 | 0.487454 | 1.15E−10 | Chr15 | 25909368 |
| rs11630828 | rs2871875 | 0.816747 | 0.414639 | 2.30E−08 | Chr15 | 25911161 |
| rs7178315 | rs2871875 | 0.845108 | 0.489457 | 5.07E−11 | Chr15 | 25911504 |
| rs735067 | rs2871875 | 0.841242 | 0.486484 | 1.51E−10 | Chr15 | 25912497 |
| rs2015343 | rs2871875 | 0.845108 | 0.489457 | 5.07E−11 | Chr15 | 25912896 |
| rs8029026 | rs2871875 | 0.845108 | 0.489457 | 5.07E−11 | Chr15 | 25913305 |
| rs2077596 | rs2871875 | 0.845108 | 0.489457 | 5.07E−11 | Chr15 | 25913330 |
| rs8024822 | rs2871875 | 0.65501 | 0.388073 | 1.52E−08 | Chr15 | 25913899 |
| rs11636259 | rs2871875 | 0.845108 | 0.489457 | 5.07E−11 | Chr15 | 25920585 |
| rs2871875 | rs2871875 | 1 | 1 | 0 | Chr15 | 25938449 |
| rs746861 | rs2871875 | 1 | 0.229167 | 1.98E−09 | Chr15 | 25939830 |
| rs2122005 | rs2871875 | 0.540118 | 0.227031 | 0.000051 | Chr15 | 25940622 |
| rs749846 | rs7170869 | 0.918367 | 0.779746 | 1.71E−15 | Chr15 | 25942585 |
| rs3794606 | rs7170869 | 0.912536 | 0.489921 | 1.50E−11 | Chr15 | 25942603 |
| rs12441727 | rs7170869 | 0.867415 | 0.436401 | 5.86E−08 | Chr15 | 25945370 |
| rs3794604 | rs7170869 | 0.874214 | 0.469173 | 7.15E−09 | Chr15 | 25945660 |
| rs3794603 | rs7170869 | 0.872051 | 0.467937 | 8.92E−09 | Chr15 | 25945919 |
| rs4778231 | rs7170869 | 0.874214 | 0.469173 | 7.15E−09 | Chr15 | 25949626 |
| rs972335 | rs7170869 | 0.874214 | 0.469173 | 7.15E−09 | Chr15 | 25950596 |
| rs4778232 | rs7170869 | 0.91342 | 0.519492 | 5.89E−12 | Chr15 | 25955360 |
| rs17680684 | rs7170869 | 0.874214 | 0.469173 | 7.15E−09 | Chr15 | 25955691 |
| rs1448485 | rs7170869 | 1 | 0.764089 | 1.59E−14 | Chr15 | 25956336 |
| rs16950821 | rs7170869 | 1 | 0.6139 | 1.39E−11 | Chr15 | 25957102 |
| rs8024968 | rs7170869 | 1 | 0.6139 | 1.39E−11 | Chr15 | 25957284 |
| rs7166228 | rs7170869 | 1 | 0.329193 | 1.37E−06 | Chr15 | 25959712 |
| rs12324648 | rs7170869 | 1 | 0.585635 | 1.48E−10 | Chr15 | 25960388 |
| rs8040696 | rs7170869 | 1 | 0.329193 | 1.37E−06 | Chr15 | 25960589 |
| rs8037142 | rs7170869 | 1 | 0.329193 | 1.50E−06 | Chr15 | 25960620 |
| rs7177686 | rs7170869 | 1 | 0.622642 | 1.09E−14 | Chr15 | 25960939 |
| rs1470608 | rs7170869 | 1 | 1 | 1.43E−20 | Chr15 | 25961716 |
| rs6497253 | rs7170869 | 1 | 0.617834 | 1.41E−14 | Chr15 | 25962144 |
| rs7170869 | rs7170869 | 1 | 1 | 0 | Chr15 | 25962343 |
| rs1375164 | rs7170869 | 1 | 0.622642 | 1.09E−14 | Chr15 | 25965407 |
| rs12442147 | rs7170869 | 1 | 0.84127 | 3.43E−16 | Chr15 | 25965773 |
| rs7163354 | rs7170869 | 1 | 0.622642 | 1.09E−14 | Chr15 | 25967383 |
| rs1597196 | rs7170869 | 0.587912 | 0.296729 | 7.75E−07 | Chr15 | 25968517 |
| rs6497254 | rs7170869 | 1 | 0.622642 | 1.09E−14 | Chr15 | 25970020 |
| rs895829 | rs7170869 | 1 | 0.588336 | 3.19E−14 | Chr15 | 25971652 |
| rs6497256 | rs7170869 | 1 | 0.588336 | 3.19E−14 | Chr15 | 25973011 |
| rs1562587 | rs7170869 | 1 | 0.923858 | 6.67E−19 | Chr15 | 25976547 |
| rs7174027 | rs7495174 | 0.823004 | 0.575481 | 2.72E−07 | Chr15 | 26002360 |
| rs12593163 | rs7495174 | 0.819616 | 0.661421 | 6.43E−07 | Chr15 | 26003963 |
| rs4778236 | rs7495174 | 0.823004 | 0.575481 | 2.72E−07 | Chr15 | 26006128 |
| rs7495174 | rs7495174 | 1 | 1 | 0 | Chr15 | 26017833 |
| rs12593929 | rs16950987 | 1 | 0.865169 | 6.76E−12 | Chr15 | 26032853 |
| rs12913832 | rs1667394 | 1 | 0.584615 | 4.27E−15 | Chr15 | 26039213 |
| rs7183877 | rs7183877 | 1 | 1 | 0 | Chr15 | 26039328 |
| rs3935591 | rs8039195 | 1 | 1 | 1.42E−19 | Chr15 | 26047607 |
| rs8025035 | rs16950987 | 1 | 0.867257 | 5.98E−12 | Chr15 | 26051367 |
| rs7497759 | rs16950987 | 1 | 1 | 2.94E−13 | Chr15 | 26089800 |
| rs12591531 | rs7183877 | 1 | 1 | 1.15E−10 | Chr15 | 26101511 |
| rs7170852 | rs8039195 | 1 | 0.919192 | 4.84E−18 | Chr15 | 26101581 |
| rs6497287 | rs7183877 | 1 | 1 | 5.11E−12 | Chr15 | 26113882 |
| rs8041209 | rs16950987 | 1 | 1 | 2.77E−13 | Chr15 | 26117253 |
| rs2238289 | rs8039195 | 1 | 0.919893 | 4.39E−17 | Chr15 | 26126810 |
| rs8182028 | rs16950979 | 1 | 1 | 5.11E−12 | Chr15 | 26141530 |
| rs8182077 | rs16950979 | 1 | 1 | 5.39E−12 | Chr15 | 26141565 |
| rs3940272 | rs8039195 | 1 | 0.918427 | 5.49E−18 | Chr15 | 26142318 |
| rs12592363 | rs16950979 | 1 | 1 | 5.39E−12 | Chr15 | 26160924 |
| rs8028689 | rs16950987 | 1 | 1 | 2.77E−13 | Chr15 | 26162483 |
| rs16950927 | rs16950979 | 1 | 1 | 5.11E−12 | Chr15 | 26163963 |
| rs2240204 | rs16950979 | 1 | 1 | 5.11E−12 | Chr15 | 26167627 |
| rs2240203 | rs16950987 | 1 | 1 | 2.77E−13 | Chr15 | 26167797 |
| rs6497292 | rs16950987 | 1 | 1 | 3.33E−13 | Chr15 | 26169790 |
| rs11631797 | rs8039195 | 1 | 1 | 1.26E−19 | Chr15 | 26175874 |
| rs16950941 | rs16950979 | 1 | 1 | 5.11E−12 | Chr15 | 26176339 |
| rs16950949 | rs7183877 | 1 | 1 | 5.11E−12 | Chr15 | 26180428 |
| rs2240202 | rs16950979 | 1 | 1 | 5.11E−12 | Chr15 | 26184490 |
| rs916977 | rs1667394 | 1 | 1 | 2.79E−22 | Chr15 | 26186959 |
| rs8039195 | rs8039195 | 1 | 1 | 0 | Chr15 | 26189679 |
| rs2016277 | rs16950987 | 1 | 1 | 2.77E−13 | Chr15 | 26191564 |
| rs2016236 | rs16950987 | 1 | 1 | 2.77E−13 | Chr15 | 26192164 |
| rs16950979 | rs16950979 | 1 | 1 | 0 | Chr15 | 26194101 |
| rs2346051 | rs16950979 | 1 | 1 | 5.39E−12 | Chr15 | 26196197 |
| rs2346050 | rs16950987 | 1 | 1 | 2.94E−13 | Chr15 | 26196279 |
| rs16950987 | rs16950987 | 1 | 1 | 0 | Chr15 | 26199823 |
| rs1667394 | rs1667394 | 1 | 1 | 0 | Chr15 | 26203777 |
| rs12592730 | rs16950979 | 1 | 1 | 5.11E−12 | Chr15 | 26203954 |
| rs16950993 | rs16950987 | 1 | 1 | 3.13E−13 | Chr15 | 26205715 |
| rs1635168 | rs16950987 | 1 | 0.866221 | 6.35E−12 | Chr15 | 26208861 |
| rs3932767 | rs16950979 | 1 | 1 | 5.11E−12 | Chr15 | 26234644 |
| rs8030727 | rs1165740 | 0.922303 | 0.734815 | 2.33E−15 | Chr15 | 27004354 |
| rs8026324 | rs7165740 | 1 | 0.433628 | 1.11E−08 | Chr15 | 27010574 |
| rs8025972 | rs7165740 | 0.92233 | 0.736314 | 1.76E−15 | Chr15 | 27013177 |
| rs6493239 | rs7165740 | 0.92233 | 0.736314 | 1.76E−15 | Chr15 | 27014674 |
| rs8035421 | rs7165740 | 0.922303 | 0.734815 | 2.33E−15 | Chr15 | 27017086 |
| rs11070769 | rs7165740 | 1 | 0.397403 | 1.51E−07 | Chr15 | 27018152 |
| rs7161763 | rs7165740 | 0.64775 | 0.27047 | 6.28E−06 | Chr15 | 27021995 |
| rs1540642 | rs7165740 | 0.92224 | 0.730218 | 2.91E−15 | Chr15 | 27022156 |
| rs1540641 | rs7165740 | 0.657143 | 0.274805 | 4.92E−06 | Chr15 | 27022218 |
| rs1540640 | rs7165740 | 0.657143 | 0.274805 | 4.92E−06 | Chr15 | 27022371 |
| rs4591103 | rs7165740 | 0.654842 | 0.273743 | 5.23E−06 | Chr15 | 27022571 |
| rs8024272 | rs7165740 | 0.92233 | 0.736314 | 1.76E−15 | Chr15 | 27022620 |
| rs17680859 | rs7165740 | 0.657143 | 0.274805 | 4.92E−06 | Chr15 | 27023010 |
| rs902269 | rs7165740 | 0.92233 | 0.736314 | 1.76E−15 | Chr15 | 27023431 |
| rs902270 | rs7165740 | 0.657143 | 0.274805 | 4.92E−06 | Chr15 | 27023525 |
| rs1565410 | rs7165740 | 0.922308 | 0.734826 | 1.99E−15 | Chr15 | 27023559 |
| rs2336907 | rs7165740 | 0.673469 | 0.304445 | 2.68E−06 | Chr15 | 27023580 |
| rs1565409 | rs7165740 | 1 | 0.433628 | 1.18E−08 | Chr15 | 27023636 |
| rs2336908 | rs7165740 | 0.657143 | 0.274805 | 4.92E−06 | Chr15 | 27023666 |
| rs2336909 | rs7165740 | 0.65251 | 0.272667 | 5.56E−06 | Chr15 | 27023714 |
| rs902271 | rs7165740 | 0.657143 | 0.274805 | 4.92E−06 | Chr15 | 27023816 |
| rs1565407 | rs7165740 | 1 | 0.433628 | 1.11E−08 | Chr15 | 27023885 |
| rs902272 | rs7165740 | 0.52381 | 0.213404 | 0.000036 | Chr15 | 27023967 |
| rs7175249 | rs7165740 | 0.699687 | 0.376151 | 7.76E−07 | Chr15 | 27024350 |
| rs1565406 | rs7165740 | 0.92233 | 0.736314 | 1.76E−15 | Chr15 | 27024713 |
| rs12324342 | rs7165740 | 0.92233 | 0.736314 | 1.76E−15 | Chr15 | 27025400 |
| rs12324117 | rs7165740 | 0.92233 | 0.736314 | 1.76E−15 | Chr15 | 27025494 |
| rs12324794 | rs7165740 | 0.92233 | 0.736314 | 1.76E−15 | Chr15 | 27025631 |
| rs8029144 | rs7165740 | 1 | 0.5 | 6.88E−10 | Chr15 | 27025865 |
| rs4779941 | rs7165740 | 0.92233 | 0.736314 | 1.76E−15 | Chr15 | 27026766 |
| rs4779942 | rs7165740 | 0.92233 | 0.736314 | 1.76E−15 | Chr15 | 27026802 |
| rs17680945 | rs7165740 | 1 | 0.433628 | 1.11E−08 | Chr15 | 27027109 |
| rs17680958 | rs7165740 | 0.922308 | 0.734826 | 1.99E−15 | Chr15 | 27027283 |
| rs17748437 | rs7165740 | 0.92233 | 0.736314 | 1.76E−15 | Chr15 | 27028128 |
| rs4300616 | rs7165740 | 0.736264 | 0.461025 | 1.99E−09 | Chr15 | 27028868 |
| rs2336912 | rs7165740 | 0.736264 | 0.461025 | 1.99E−09 | Chr15 | 27029521 |
| rs12439742 | rs7165740 | 0.734317 | 0.459732 | 2.17E−09 | Chr15 | 27030510 |
| rs17680978 | rs1907001 | 1 | 0.808 | 9.23E−18 | Chr15 | 27032422 |
| rs7168529 | rs7165740 | 1 | 0.241379 | 0.000027 | Chr15 | 27034321 |
| rs16954833 | rs7165740 | 1 | 0.706422 | 7.47E−14 | Chr15 | 27035216 |
| rs7170159 | rs7165740 | 1 | 0.259259 | 0.000021 | Chr15 | 27035295 |
| rs4779594 | rs7165740 | 1 | 0.706422 | 6.77E−14 | Chr15 | 27035760 |
| rs8035791 | rs7165740 | 1 | 0.304348 | 2.23E−06 | Chr15 | 27035781 |
| rs11854795 | rs1907001 | 1 | 1 | 2.20E−21 | Chr15 | 27037285 |
| rs8026832 | rs1907001 | 1 | 1 | 2.20E−21 | Chr15 | 27038893 |
| rs8043183 | rs7165740 | 1 | 0.241379 | 0.000026 | Chr15 | 27038989 |
| rs8035481 | rs7165740 | 1 | 0.368421 | 1.61E−07 | Chr15 | 27040249 |
| rs7181300 | rs1907001 | 1 | 1 | 2.20E−21 | Chr15 | 27041180 |
| rs10162860 | rs7165740 | 0.754232 | 0.525933 | 2.02E−10 | Chr15 | 27043036 |
| rs1873285 | rs7165740 | 0.754232 | 0.525933 | 2.02E−10 | Chr15 | 27043388 |
| rs2171894 | rs7165740 | 1 | 0.304348 | 2.13E−06 | Chr15 | 27043450 |
| rs13379809 | rs7165740 | 0.754232 | 0.525933 | 2.02E−10 | Chr15 | 27046714 |
| rs4424881 | rs1907001 | 1 | 0.806452 | 1.06E−17 | Chr15 | 27049008 |
| rs7171892 | rs7165740 | 1 | 0.241379 | 0.000026 | Chr15 | 27051798 |
| rs17748495 | rs7165740 | 1 | 0.706422 | 6.77E−14 | Chr15 | 27053463 |
| rs1907001 | rs1907001 | 1 | 1 | 0 | Chr15 | 27053851 |
| rs7182415 | rs7165740 | 1 | 0.241379 | 0.000026 | Chr15 | 27053934 |
| rs7179243 | rs1907001 | 1 | 1 | 2.20E−21 | Chr15 | 27054615 |
| rs7162303 | rs7165740 | 1 | 0.241379 | 0.000026 | Chr15 | 27055170 |
| rs7164360 | rs7165740 | 1 | 0.706422 | 6.77E−14 | Chr15 | 27055842 |
| rs1490081 | rs7165740 | 1 | 0.304348 | 2.13E−06 | Chr15 | 27056024 |
| rs12443112 | rs1907001 | 1 | 1 | 2.20E−21 | Chr15 | 27056973 |
| rs7165740 | rs7165740 | 1 | 1 | 0 | Chr15 | 27057792 |
| rs12437939 | rs1907001 | 1 | 1 | 2.20E−21 | Chr15 | 27058305 |
| rs8033160 | rs7165740 | 1 | 0.241379 | 0.000026 | Chr15 | 27061851 |
| rs6495774 | rs7165740 | 1 | 0.919192 | 7.05E−18 | Chr15 | 27062116 |
| rs8035785 | rs7165740 | 1 | 0.241379 | 0.000026 | Chr15 | 27063043 |
| rs8025184 | rs1907001 | 1 | 1 | 2.88E−21 | Chr15 | 27063146 |
| rs937048 | rs1907001 | 0.832394 | 0.301602 | 3.60E−06 | Chr15 | 27071058 |
| rs937049 | rs7165740 | 0.820225 | 0.263441 | 0.000022 | Chr15 | 27071206 |
| rs937050 | rs7165740 | 0.836735 | 0.303594 | 2.91E−06 | Chr15 | 27071351 |
| rs4624139 | rs7165740 | 0.809524 | 0.241437 | 0.000033 | Chr15 | 27071533 |
| rs2878830 | rs1907001 | 1 | 0.926606 | 1.11E−18 | Chr15 | 27073796 |
| rs2878831 | rs7165740 | 0.836735 | 0.303594 | 2.91E−06 | Chr15 | 27074124 |
| rs4995092 | rs7165740 | 0.836735 | 0.303594 | 2.91E−06 | Chr15 | 27074190 |
| rs4995091 | rs7165740 | 0.836735 | 0.303594 | 2.91E−06 | Chr15 | 27074231 |
| rs4995090 | rs7165740 | 0.836735 | 0.303594 | 2.91E−06 | Chr15 | 27074320 |
| rs3812917 | rs7165740 | 0.835391 | 0.303061 | 3.06E−06 | Chr15 | 27075314 |
| rs10519643 | rs7165740 | 0.836735 | 0.303594 | 2.91E−06 | Chr15 | 27075712 |
| rs10775229 | rs7165740 | 0.843575 | 0.329634 | 1.96E−06 | Chr15 | 27076604 |
| rs10775230 | rs7165740 | 0.836735 | 0.303594 | 2.91E−06 | Chr15 | 27076765 |
| rs750393 | rs7165740 | 0.836735 | 0.303594 | 2.91E−06 | Chr15 | 27077766 |
| rs4779669 | rs1907001 | 1 | 0.928571 | 9.66E−20 | Chr15 | 27080616 |
| rs1994597 | rs1907001 | 1 | 0.928571 | 9.66E−20 | Chr15 | 27081472 |
| rs7178120 | rs1907001 | 1 | 0.865546 | 1.12E−18 | Chr15 | 27082769 |
| rs6495913 | rs1907001 | 1 | 0.928571 | 9.66E−20 | Chr15 | 27083162 |
| rs2046832 | rs7165740 | 0.835391 | 0.303061 | 3.06E−06 | Chr15 | 27083797 |
| rs16954986 | rs7165740 | 1 | 0.567568 | 3.75E−11 | Chr15 | 27084142 |
| rs12148594 | rs7165740 | 0.820225 | 0.263441 | 0.000022 | Chr15 | 27084151 |
| rs2087535 | rs7165740 | 1 | 0.567568 | 3.75E−11 | Chr15 | 27085875 |
| rs7164987 | rs7165740 | 0.836735 | 0.303594 | 2.91E−06 | Chr15 | 27086304 |
| rs4780261 | rs7165740 | 1 | 0.567568 | 3.75E−11 | Chr15 | 27087052 |
| rs4780262 | rs7165740 | 1 | 0.567568 | 3.75E−11 | Chr15 | 27087150 |
| rs1490078 | rs1907001 | 1 | 0.757576 | 4.30E−17 | Chr15 | 27090823 |
| rs1490079 | rs7165740 | 1 | 0.567568 | 3.75E−11 | Chr15 | 27091095 |
| rs923529 | rs7165740 | 1 | 0.567568 | 3.75E−11 | Chr15 | 27091288 |
| rs12441482 | rs1907001 | 1 | 0.927928 | 8.50E−19 | Chr15 | 27092665 |
| rs16954991 | rs7165740 | 1 | 0.567568 | 3.75E−11 | Chr15 | 27092729 |
| rs1490080 | rs1907001 | 1 | 0.928571 | 9.66E−20 | Chr15 | 27095196 |
| rs4779678 | rs12441723 | 0.861587 | 0.694285 | 1.40E−14 | Chr15 | 27097183 |
| rs4779679 | rs12441723 | 0.930151 | 0.814604 | 3.40E−17 | Chr15 | 27097223 |
| rs4779680 | rs12441723 | 0.933993 | 0.818328 | 5.56E−18 | Chr15 | 27097430 |
| rs1565404 | rs12441723 | 0.93398 | 0.817293 | 6.49E−18 | Chr15 | 27097955 |
| rs1565405 | rs12441723 | 0.933993 | 0.818328 | 5.56E−18 | Chr15 | 27098012 |
| rs7181045 | rs12441723 | 0.929783 | 0.812962 | 1.61E−16 | Chr15 | 27102064 |
| rs7176462 | rs12441723 | 0.931896 | 0.814634 | 4.80E−17 | Chr15 | 27102074 |
| rs11070208 | rs7165740 | 1 | 0.686275 | 5.68E−13 | Chr15 | 27102607 |
| rs11070209 | rs12441723 | 0.933993 | 0.818328 | 5.56E−18 | Chr15 | 27102636 |
| rs4779689 | rs12441723 | 0.933993 | 0.818328 | 5.56E−18 | Chr15 | 27102896 |
| rs4779469 | rs12441723 | 0.933993 | 0.818328 | 5.56E−18 | Chr15 | 27103086 |
| rs8031475 | rs12441723 | 0.933993 | 0.818328 | 5.56E−18 | Chr15 | 27103758 |
| rs4779471 | rs7165740 | 1 | 0.567568 | 4.06E−11 | Chr15 | 27104029 |
| rs4779472 | rs12441723 | 0.933993 | 0.818328 | 5.56E−18 | Chr15 | 27104170 |
| rs4779473 | rs12441723 | 0.933993 | 0.818328 | 5.56E−18 | Chr15 | 27104187 |
| rs4779692 | rs12441723 | 0.933993 | 0.818328 | 5.56E−18 | Chr15 | 27104303 |
| rs4779474 | rs12441723 | 0.933993 | 0.818328 | 5.56E−18 | Chr15 | 27104395 |
| rs4779694 | rs12441723 | 0.933993 | 0.818328 | 5.56E−18 | Chr15 | 27104752 |
| rs12437861 | rs12441723 | 0.933993 | 0.818328 | 5.56E−18 | Chr15 | 27105142 |
| rs7178921 | rs7165740 | 1 | 0.368421 | 1.61E−07 | Chr15 | 27105836 |
| rs7183691 | rs12441723 | 0.933993 | 0.818328 | 5.56E−18 | Chr15 | 27105940 |
| rs7164138 | rs12441723 | 1 | 0.571865 | 2.01E−12 | Chr15 | 27110565 |
| rs4779705 | rs7165740 | 1 | 0.368421 | 1.61E−07 | Chr15 | 27113158 |
| rs2077778 | rs12441723 | 1 | 0.571865 | 2.01E−12 | Chr15 | 27114644 |
| rs11853379 | rs12441723 | 1 | 0.571865 | 2.01E−12 | Chr15 | 27117346 |
| rs17748747 | rs12441723 | 1 | 0.935275 | 2.52E−21 | Chr15 | 27119190 |
| rs12438928 | rs12441723 | 1 | 0.935275 | 2.16E−21 | Chr15 | 27119567 |
| rs12442688 | rs12441723 | 1 | 0.935275 | 2.52E−21 | Chr15 | 27119687 |
| rs12443349 | rs12441723 | 1 | 0.935275 | 2.16E−21 | Chr15 | 27119878 |
| rs12441723 | rs12441723 | 1 | 1 | 0 | Chr15 | 27120318 |
| rs12439789 | rs12441723 | 1 | 0.935275 | 2.16E−21 | Chr15 | 27120492 |
| rs12438284 | rs12441723 | 1 | 0.935275 | 2.16E−21 | Chr15 | 27121583 |
| rs7183087 | rs12441723 | 1 | 0.935275 | 3.44E−21 | Chr15 | 27122052 |
| rs7173909 | rs12441723 | 1 | 0.935275 | 2.16E−21 | Chr15 | 27122255 |
| rs17748777 | rs12441723 | 1 | 0.935275 | 2.16E−21 | Chr15 | 27122766 |
| rs8028477 | rs12441723 | 1 | 0.935275 | 2.16E−21 | Chr15 | 27123897 |
| rs1873280 | rs1907001 | 0.473849 | 0.201783 | 0.000041 | Chr15 | 27125501 |
| rs7176361 | rs16950987 | 1 | 0.273608 | 0.000541 | Chr15 | 27413773 |
| rs7176689 | rs16950987 | 1 | 0.273608 | 0.000551 | Chr15 | 27428369 |
| rs16955366 | rs16950987 | 1 | 0.273608 | 0.000541 | Chr15 | 27428661 |
| rs16955368 | rs16950987 | 1 | 0.320113 | 0.00037 | Chr15 | 27432392 |
| rs8039745 | rs16950987 | 1 | 0.273608 | 0.000551 | Chr15 | 27432568 |
| rs4238678 | rs3212346 | 1 | 0.20904 | 0.00101 | Chr16 | 87267745 |
| rs4782321 | rs4782509 | 0.91421 | 0.229106 | 4.58E−08 | Chr16 | 87307676 |
| rs2242171 | rs4782509 | 0.55809 | 0.281241 | 1.54E−08 | Chr16 | 87321411 |
| rs750740 | rs4782509 | 0.5246 | 0.210562 | 1.50E−06 | Chr16 | 87334839 |
| rs750739 | rs4782509 | 0.861706 | 0.258386 | 6.46E−09 | Chr16 | 87335109 |
| rs9933845 | rs4782509 | 0.546768 | 0.232184 | 7.59E−07 | Chr16 | 87337394 |
| rs8062328 | rs8062328 | 1 | 1 | 0 | Chr16 | 87343542 |
| rs3843706 | rs8062328 | 0.859026 | 0.398143 | 1.13E−10 | Chr16 | 87348534 |
| rs8047820 | rs8062328 | 0.866059 | 0.427588 | 2.96E−11 | Chr16 | 87351166 |
| rs2278053 | rs4782509 | 1 | 0.33358 | 5.92E−13 | Chr16 | 87353574 |
| rs4782509 | rs4782509 | 1 | 1 | 0 | Chr16 | 87354279 |
| rs7203655 | rs4782509 | 1 | 0.68031 | 4.38E−24 | Chr16 | 87354363 |
| rs17666981 | rs4782509 | 0.94836 | 0.442565 | 3.91E−14 | Chr16 | 87354710 |
| rs6500501 | rs4782509 | 1 | 0.656078 | 5.63E−23 | Chr16 | 87356917 |
| rs6500503 | rs4782509 | 0.889415 | 0.525643 | 1.43E−13 | Chr16 | 87358106 |
| rs17176204 | rs8062328 | 0.90966 | 0.335577 | 9.91E−09 | Chr16 | 87358337 |
| rs889764 | rs4782509 | 0.906666 | 0.459903 | 2.43E−14 | Chr16 | 87360910 |
| rs17176239 | rs4782509 | 0.72724 | 0.242212 | 8.97E−08 | Chr16 | 87369881 |
| rs2968481 | rs4782509 | 0.884779 | 0.345819 | 1.76E−10 | Chr16 | 87371898 |
| rs2911457 | rs4782509 | 0.88928 | 0.349346 | 2.27E−11 | Chr16 | 87372602 |
| rs2926771 | rs4782509 | 0.934728 | 0.30247 | 1.81E−10 | Chr16 | 87372679 |
| rs8043839 | rs4782509 | 0.907253 | 0.465082 | 1.40E−14 | Chr16 | 87374938 |
| rs8044021 | rs4782509 | 0.950591 | 0.520019 | 5.57E−15 | Chr16 | 87375038 |
| rs558436 | rs4782509 | 0.891063 | 0.35869 | 1.35E−11 | Chr16 | 87375182 |
| rs860762 | rs4782509 | 0.879749 | 0.306118 | 4.79E−10 | Chr16 | 87383871 |
| rs477639 | rs4782497 | 1 | 0.20861 | 4.94E−09 | Chr16 | 87517011 |
| rs531778 | rs4782497 | 0.695488 | 0.272015 | 5.43E−07 | Chr16 | 87519213 |
| rs545448 | rs4782497 | 0.807732 | 0.412928 | 1.14E−10 | Chr16 | 87536011 |
| rs12935775 | rs4782497 | 1 | 0.230047 | 1.44E−09 | Chr16 | 87542372 |
| rs12103116 | rs4782497 | 1 | 0.230047 | 8.38E−10 | Chr16 | 87543875 |
| rs4782497 | rs4782497 | 1 | 1 | 0 | Chr16 | 87546780 |
| rs4782496 | rs4782497 | 1 | 0.212938 | 2.82E−09 | Chr16 | 87547354 |
| rs8043943 | rs4782497 | 0.913766 | 0.206093 | 2.40E−07 | Chr16 | 87553097 |
| rs12447686 | rs11076747 | 0.96287 | 0.893949 | 1.40E−28 | Chr16 | 87575792 |
| rs11076744 | rs11076747 | 0.963358 | 0.928059 | 4.79E−30 | Chr16 | 87576985 |
| rs12446471 | rs9932354 | 1 | 1 | 1.44E−35 | Chr16 | 87577658 |
| rs11076745 | rs9932354 | 1 | 1 | 8.05E−37 | Chr16 | 87578590 |
| rs9932354 | rs9932354 | 1 | 1 | 0 | Chr16 | 87580066 |
| rs7404396 | rs9932354 | 1 | 0.760897 | 1.68E−26 | Chr16 | 87580839 |
| rs9924109 | rs9932354 | 1 | 1 | 1.33E−36 | Chr16 | 87581788 |
| rs11639542 | rs9932354 | 1 | 1 | 3.41E−36 | Chr16 | 87583532 |
| rs12444247 | rs9932354 | 1 | 0.965288 | 1.12E−33 | Chr16 | 87584122 |
| rs11076747 | rs11076747 | 1 | 1 | 0 | Chr16 | 87584526 |
| rs4782485 | rs9932354 | 1 | 0.965497 | 1.66E−34 | Chr16 | 87584788 |
| rs6500514 | rs9932354 | 1 | 1 | 1.33E−36 | Chr16 | 87585062 |
| rs9926548 | rs11076747 | 1 | 1 | 3.31E−35 | Chr16 | 87585231 |
| rs8044191 | rs9932354 | 1 | 0.965497 | 1.16E−33 | Chr16 | 87586141 |
| rs11644990 | rs11076747 | 1 | 0.962963 | 1.12E−32 | Chr16 | 87586886 |
| rs8056679 | rs11076747 | 1 | 0.89418 | 4.71E−30 | Chr16 | 87588754 |
| rs4782481 | rs7498845 | 1 | 1 | 2.10E−36 | Chr16 | 87591002 |
| rs7498845 | rs7498845 | 1 | 1 | 0 | Chr16 | 87594028 |
| rs9939076 | rs7498845 | 0.964185 | 0.928731 | 2.47E−30 | Chr16 | 87607274 |
| rs13336078 | rs7498845 | 0.964444 | 0.930153 | 4.46E−31 | Chr16 | 87608419 |
| rs11639948 | rs7498845 | 0.964444 | 0.930153 | 4.46E−31 | Chr16 | 87612298 |
| rs12446899 | rs7498845 | 0.964438 | 0.929694 | 7.06E−31 | Chr16 | 87613016 |
| rs11076752 | rs7498845 | 0.963708 | 0.928266 | 3.91E−30 | Chr16 | 87613669 |
| rs9939105 | rs7498845 | 0.768302 | 0.495842 | 2.18E−14 | Chr16 | 87660273 |
| rs3934812 | rs12599126 | 0.55289 | 0.254277 | 5.99E−07 | Chr16 | 87674956 |
| rs4782468 | rs12599126 | 0.671304 | 0.450649 | 1.66E−11 | Chr16 | 87690766 |
| rs7188200 | rs12599126 | 0.619099 | 0.288317 | 5.25E−07 | Chr16 | 87694595 |
| rs7200676 | rs12599126 | 0.714771 | 0.507171 | 3.13E−12 | Chr16 | 87696412 |
| rs4782456 | rs12599126 | 0.634614 | 0.297061 | 2.62E−07 | Chr16 | 87696706 |
| rs4782455 | rs12599126 | 0.719517 | 0.517705 | 7.44E−13 | Chr16 | 87696813 |
| rs6500531 | rs12599126 | 0.719517 | 0.517705 | 7.44E−13 | Chr16 | 87697015 |
| rs4782453 | rs12599126 | 0.70473 | 0.483061 | 9.19E−11 | Chr16 | 87697548 |
| rs7184784 | rs12599126 | 0.715723 | 0.508535 | 1.58E−12 | Chr16 | 87697672 |
| rs8044145 | rs12599126 | 0.796447 | 0.611284 | 2.52E−14 | Chr16 | 87698180 |
| rs7187583 | rs12599126 | 0.9507 | 0.723213 | 6.36E−20 | Chr16 | 87700761 |
| rs4782449 | rs12599126 | 0.807329 | 0.569508 | 7.46E−15 | Chr16 | 87706274 |
| rs9940965 | rs3751688 | 0.700262 | 0.326546 | 0.000025 | Chr16 | 87708165 |
| rs3743979 | rs12599126 | 0.717283 | 0.491354 | 1.58E−12 | Chr16 | 87708384 |
| rs3743980 | rs12599126 | 0.848511 | 0.603463 | 1.55E−15 | Chr16 | 87708436 |
| rs4782328 | rs12599126 | 0.618207 | 0.2875 | 2.20E−06 | Chr16 | 87709719 |
| rs4782445 | rs12599126 | 0.807329 | 0.569508 | 7.46E−15 | Chr16 | 87709796 |
| rs8052780 | rs12599126 | 0.727502 | 0.363577 | 8.76E−08 | Chr16 | 87710427 |
| rs8053537 | rs12599126 | 0.67135 | 0.370226 | 6.18E−09 | Chr16 | 87710698 |
| rs4782327 | rs12599126 | 0.682268 | 0.403841 | 9.20E−10 | Chr16 | 87711636 |
| rs12443551 | rs12599126 | 0.751363 | 0.317974 | 3.99E−06 | Chr16 | 87712520 |
| rs3743983 | rs12599126 | 0.761813 | 0.529893 | 1.20E−13 | Chr16 | 87714879 |
| rs7500326 | rs12599126 | 1 | 0.505495 | 7.25E−14 | Chr16 | 87715455 |
| rs7498787 | rs12599126 | 1 | 0.505495 | 7.25E−14 | Chr16 | 87716973 |
| rs8050455 | rs12599126 | 0.803848 | 0.544071 | 3.91E−13 | Chr16 | 87718639 |
| rs8059060 | rs885479 | 0.716389 | 0.309515 | 7.22E−06 | Chr16 | 87718765 |
| rs4782437 | rs12599126 | 0.805566 | 0.590153 | 6.11E−15 | Chr16 | 87718798 |
| rs8049456 | rs12599126 | 1 | 0.845824 | 2.26E−21 | Chr16 | 87721751 |
| rs11646135 | rs12599126 | 1 | 0.618105 | 6.94E−17 | Chr16 | 87726609 |
| rs4530136 | rs12599126 | 1 | 0.69697 | 4.14E−19 | Chr16 | 87729306 |
| rs12934720 | rs12599126 | 1 | 0.517169 | 1.29E−13 | Chr16 | 87729542 |
| rs12930806 | rs12599126 | 1 | 0.471489 | 1.52E−10 | Chr16 | 87730574 |
| rs11076770 | rs12599126 | 1 | 0.579832 | 7.68E−16 | Chr16 | 87733011 |
| rs10852635 | rs12599126 | 1 | 0.579832 | 7.68E−16 | Chr16 | 87733374 |
| rs12599126 | rs12599126 | 1 | 1 | 0 | Chr16 | 87733984 |
| rs12931350 | rs12599126 | 0.924202 | 0.509867 | 2.05E−11 | Chr16 | 87735816 |
| rs12325574 | rs12599126 | 1 | 0.954545 | 1.48E−27 | Chr16 | 87735835 |
| rs12917851 | rs12599126 | 0.918526 | 0.469913 | 1.84E−10 | Chr16 | 87735908 |
| rs12917772 | rs12599126 | 0.93944 | 0.445128 | 1.61E−12 | Chr16 | 87742473 |
| rs1054747 | rs9921361 | 1 | 0.557522 | 1.29E−09 | Chr16 | 87748347 |
| rs12598678 | rs9921361 | 0.79798 | 0.578352 | 2.39E−10 | Chr16 | 87750726 |
| rs883941 | rs12599126 | 1 | 1 | 3.37E−30 | Chr16 | 87754012 |
| rs11640375 | rs12599126 | 1 | 1 | 1.91E−29 | Chr16 | 87755318 |
| rs4352046 | rs12599126 | 1 | 1 | 3.37E−30 | Chr16 | 87756589 |
| rs4581700 | rs12599126 | 1 | 0.69697 | 4.14E−19 | Chr16 | 87758090 |
| rs12600036 | rs12599126 | 1 | 0.707674 | 3.37E−21 | Chr16 | 87758486 |
| rs12598250 | rs12599126 | 1 | 1 | 1.91E−29 | Chr16 | 87758552 |
| rs2002259 | rs9921361 | 0.887006 | 0.645054 | 3.87E−11 | Chr16 | 87761255 |
| rs12922670 | rs12599126 | 1 | 0.506539 | 7.56E−13 | Chr16 | 87762449 |
| rs729201 | rs9921361 | 1 | 0.557522 | 1.14E−09 | Chr16 | 87768284 |
| rs729202 | rs9921361 | 1 | 0.557522 | 1.14E−09 | Chr16 | 87768439 |
| rs3887583 | rs9921361 | 1 | 0.557522 | 1.14E−09 | Chr16 | 87768448 |
| rs4785578 | rs9921361 | 1 | 0.557522 | 1.29E−09 | Chr16 | 87770982 |
| rs2287358 | rs9921361 | 1 | 0.557522 | 1.14E−09 | Chr16 | 87773456 |
| rs9635539 | rs12599126 | 0.865848 | 0.298373 | 6.87E−09 | Chr16 | 87778383 |
| rs4785719 | rs12599126 | 0.854522 | 0.289411 | 2.01E−08 | Chr16 | 87783632 |
| rs3785415 | rs12599126 | 0.889005 | 0.46347 | 4.47E−13 | Chr16 | 87788064 |
| rs2270416 | rs885479 | 0.858965 | 0.629028 | 2.09E−08 | Chr16 | 87788983 |
| rs3743986 | rs12599126 | 0.87842 | 0.390307 | 1.30E−10 | Chr16 | 87791352 |
| rs8063985 | rs12599126 | 0.93092 | 0.535657 | 3.05E−13 | Chr16 | 87796353 |
| rs4998656 | rs9921361 | 0.777778 | 0.236715 | 0.000111 | Chr16 | 87803419 |
| rs12596323 | rs9921361 | 0.776119 | 0.236166 | 0.000115 | Chr16 | 87804936 |
| rs9933428 | rs9921361 | 1 | 0.310345 | 8.61E−06 | Chr16 | 87811171 |
| rs9925542 | rs9921361 | 1 | 0.252336 | 0.0001 | Chr16 | 87811365 |
| rs3803722 | rs9921361 | 1 | 0.252336 | 0.000103 | Chr16 | 87811787 |
| rs8058136 | rs9921361 | 1 | 0.310345 | 8.61E−06 | Chr16 | 87811994 |
| rs7202536 | rs9921361 | 1 | 0.310345 | 8.61E−06 | Chr16 | 87813422 |
| rs13332037 | rs9921361 | 1 | 0.310345 | 8.61E−06 | Chr16 | 87814063 |
| rs4785771 | rs9921361 | 1 | 0.673203 | 3.52E−14 | Chr16 | 87814584 |
| rs8059397 | rs9921361 | 1 | 0.310345 | 9.57E−06 | Chr16 | 87815366 |
| rs12920028 | rs9921361 | 1 | 0.252336 | 0.0001 | Chr16 | 87817125 |
| rs4785626 | rs9921361 | 1 | 0.391304 | 4.99E−07 | Chr16 | 87821115 |
| rs4785627 | rs9921361 | 1 | 0.391304 | 4.99E−07 | Chr16 | 87821685 |
| rs9921361 | rs9921361 | 1 | 1 | 0 | Chr16 | 87821940 |
| rs10400977 | rs9921361 | 1 | 0.252336 | 0.000134 | Chr16 | 87824984 |
| rs4785629 | rs9921361 | 1 | 0.557522 | 1.14E−09 | Chr16 | 87825365 |
| rs10400958 | rs9921361 | 1 | 0.310345 | 9.24E−06 | Chr16 | 87825453 |
| rs9972663 | rs9921361 | 1 | 0.310345 | 8.61E−06 | Chr16 | 87826016 |
| rs9972734 | rs9921361 | 1 | 0.310345 | 8.61E−06 | Chr16 | 87826117 |
| rs9939155 | rs9921361 | 1 | 0.878049 | 4.18E−13 | Chr16 | 87829238 |
| rs9941273 | rs9921361 | 1 | 0.603645 | 3.01E−08 | Chr16 | 87829351 |
| rs4785630 | rs9921361 | 0.898839 | 0.804485 | 7.20E−13 | Chr16 | 87831104 |
| rs4294815 | rs9921361 | 1 | 0.908257 | 3.25E−16 | Chr16 | 87832389 |
| rs9926404 | rs9921361 | 1 | 0.818182 | 4.40E−14 | Chr16 | 87832450 |
| rs4785644 | rs2353028 | 0.770095 | 0.566978 | 2.42E−15 | Chr16 | 87848739 |
| rs3096314 | rs3096304 | 0.742489 | 0.487211 | 4.81E−11 | Chf16 | 87852014 |
| rs3114860 | rs3096304 | 0.899334 | 0.478162 | 3.90E−10 | Chr16 | 87854094 |
| rs7192049 | rs455527 | 1 | 0.473684 | 2.57E−08 | Chr16 | 87854103 |
| rs4785648 | rs4785648 | 1 | 1 | 0 | Chr16 | 87855978 |
| rs4541078 | rs4785648 | 1 | 0.536456 | 5.05E−12 | Chr16 | 87859609 |
| rs4785649 | rs4785648 | 1 | 0.536456 | 8.25E−12 | Chr16 | 87859922 |
| rs12928649 | rs4785648 | 0.804537 | 0.422674 | 9.40E−09 | Chr16 | 87860843 |
| rs4238828 | rs4785648 | 1 | 0.329297 | 1.01E−07 | Chr16 | 87863319 |
| rs2306632 | rs4785648 | 1 | 0.536456 | 5.05E−12 | Chr16 | 87864644 |
| rs2883069 | rs2353028 | 1 | 0.495146 | 6.38E−14 | Chr16 | 87865917 |
| rs1542633 | rs4785648 | 1 | 0.329297 | 1.01E−07 | Chr16 | 87867000 |
| rs1542634 | rs4785648 | 1 | 0.329297 | 1.01E−07 | Chr16 | 87867072 |
| rs4785651 | rs4785648 | 0.838875 | 0.258556 | 0.000011 | Chr16 | 87867723 |
| rs1466539 | rs3096304 | 1 | 0.645844 | 1.91E−14 | Chr16 | 87871932 |
| rs1466540 | rs1466540 | 1 | 1 | 0 | Chr16 | 87871978 |
| rs2279348 | rs2353028 | 1 | 1 | 3.37E−31 | Chr16 | 87877539 |
| rs2279349 | rs2353028 | 1 | 0.495146 | 4.68E−14 | Chr16 | 87877679 |
| rs2353028 | rs2353028 | 1 | 1 | 0 | Chr16 | 87880179 |
| rs2306633 | rs2306633 | 1 | 1 | 0 | Chr16 | 87882779 |
| rs731136 | rs2306633 | 0.95116 | 0.824197 | 1.00E−22 | Chr16 | 87886922 |
| rs3102368 | rs3096304 | 0.872856 | 0.705635 | 3.91E−15 | Chr16 | 87888567 |
| rs3114916 | rs2306633 | 0.894689 | 0.734572 | 7.24E−19 | Chr16 | 87889114 |
| rs3096294 | rs2306633 | 1 | 0.906433 | 6.59E−25 | Chr16 | 87892787 |
| rs3803694 | rs3096304 | 1 | 0.238705 | 8.45E−06 | Chr16 | 87896679 |
| rs3102345 | rs3096304 | 0.936184 | 0.775924 | 5.37E−18 | Chr16 | 87896796 |
| rs3114912 | rs3096304 | 1 | 0.835165 | 1.30E−20 | Chr16 | 87899328 |
| rs2277908 | rs3096304 | 1 | 0.833718 | 1.55E−20 | Chr16 | 87899340 |
| rs3096304 | rs3096304 | 1 | 1 | 0 | Chr16 | 87901208 |
| rs3096302 | rs3096304 | 1 | 0.428257 | 2.28E−07 | Chr16 | 87902740 |
| rs3114910 | rs3096304 | 0.907757 | 0.532649 | 2.54E−11 | Chr16 | 87906928 |
| rs3102350 | rs3096304 | 1 | 0.279778 | 9.91E−07 | Chr16 | 87907437 |
| rs3102352 | rs889574 | 0.890024 | 0.213507 | 1.40E−06 | Chr16 | 87908806 |
| rs3114908 | rs2353033 | 1 | 0.534483 | 2.36E−19 | Chr16 | 87911226 |
| rs3102357 | rs2353033 | 0.825509 | 0.658572 | 5.23E−19 | Chr16 | 87912056 |
| rs2353033 | rs2353033 | 1 | 1 | 0 | Chr16 | 87913062 |
| rs889574 | rs889574 | 1 | 1 | 0 | Chr16 | 87914309 |
| rs3114898 | rs889574 | 1 | 0.64878 | 2.18E−22 | Chr16 | 87919816 |
| rs3114896 | rs889574 | 1 | 0.543828 | 2.19E−19 | Chr16 | 87921063 |
| rs11076780 | rs889574 | 1 | 0.737562 | 8.19E−24 | Chr16 | 87925455 |
| rs3102372 | rs2965946 | 0.913554 | 0.726782 | 9.56E−19 | Chr16 | 87925772 |
| rs3102373 | rs889574 | 0.957893 | 0.708684 | 2.68E−22 | Chr16 | 87926123 |
| rs3114891 | rs889574 | 1 | 0.566396 | 9.99E−20 | Chr16 | 87926956 |
| rs3102376 | rs889574 | 1 | 0.512195 | 1.49E−18 | Chr16 | 87929974 |
| rs3096319 | rs889574 | 1 | 0.525692 | 7.06E−19 | Chr16 | 87930135 |
| rs2353030 | rs2965946 | 1 | 0.684211 | 7.25E−23 | Chr16 | 87933328 |
| rs3102381 | rs889574 | 1 | 0.470811 | 5.69E−17 | Chr16 | 87937221 |
| rs3102382 | rs889574 | 1 | 0.463415 | 3.61E−17 | Chr16 | 87937243 |
| rs3102383 | rs889574 | 0.953272 | 0.550693 | 1.44E−17 | Chr16 | 87938796 |
| rs918722 | rs889574 | 1 | 0.626973 | 2.12E−21 | Chr16 | 87942582 |
| rs2035481 | rs889574 | 1 | 0.512195 | 1.49E−18 | Chr16 | 87947857 |
| rs3114881 | rs889574 | 1 | 0.582173 | 1.87E−20 | Chr16 | 87950133 |
| rs3096324 | rs889574 | 1 | 0.623552 | 1.31E−21 | Chr16 | 87950324 |
| rs889576 | rs889574 | 1 | 0.547672 | 1.49E−19 | Chr16 | 87957154 |
| rs889573 | rs2965946 | 0.552039 | 0.253956 | 1.29E−07 | Chr16 | 87965061 |
| rs744327 | rs889574 | 1 | 0.547672 | 1.49E−19 | Chr16 | 87966771 |
| rs3096322 | rs889574 | 1 | 0.64878 | 2.18E−22 | Chr16 | 87968624 |
| rs753852 | rs889574 | 1 | 0.541651 | 3.93E−19 | Chr16 | 87973920 |
| rs3096299 | rs889574 | 1 | 0.562642 | 6.55E−20 | Chr16 | 87976164 |
| rs3114848 | rs889574 | 1 | 0.554937 | 1.43E−19 | Chr16 | 87980272 |
| rs2965824 | rs889574 | 1 | 0.585826 | 1.27E−20 | Chr16 | 87982807 |
| rs2965827 | rs889574 | 1 | 0.585826 | 1.27E−20 | Chr16 | 87984851 |
| rs7205785 | rs889574 | 1 | 0.585826 | 1.27E−20 | Chr16 | 87986485 |
| rs4785666 | rs2965946 | 0.955219 | 0.576733 | 8.43E−19 | Chr16 | 87990272 |
| rs11648663 | rs889574 | 1 | 0.585826 | 1.27E−20 | Chr16 | 87993808 |
| rs2086824 | rs889574 | 1 | 0.433517 | 2.61E−16 | Chr16 | 87998747 |
| rs2911258 | rs889574 | 1 | 0.587629 | 2.91E−19 | Chr16 | 88008261 |
| rs2965935 | rs889574 | 1 | 0.582173 | 1.87E−20 | Chr16 | 88008775 |
| rs2911257 | rs889574 | 1 | 0.585826 | 1.27E−20 | Chr16 | 88011635 |
| rs2911256 | rs889574 | 1 | 0.578454 | 2.76E−20 | Chr16 | 88012597 |
| rs2911255 | rs889574 | 1 | 0.585826 | 1.27E−20 | Chr16 | 88014638 |
| rs2911253 | rs889574 | 1 | 0.585826 | 2.91E−20 | Chr16 | 88016088 |
| rs2930219 | rs889574 | 1 | 0.556671 | 3.88E−19 | Chr16 | 88019550 |
| rs2965939 | rs889574 | 1 | 0.566396 | 4.45E−20 | Chr16 | 88020544 |
| rs2965819 | rs4347628 | 0.959387 | 0.656755 | 2.62E−21 | Chr16 | 88028531 |
| rs9931120 | rs4347628 | 0.920065 | 0.624508 | 9.14E−20 | Chr16 | 88028858 |
| rs2965940 | rs382745 | 0.8857 | 0.62022 | 1.07E−19 | Chr16 | 88033941 |
| rs9302767 | rs889574 | 0.732666 | 0.46082 | 7.88E−13 | Chr16 | 88036363 |
| rs2965818 | rs4347628 | 1 | 0.734579 | 5.39E−26 | Chr16 | 88037995 |
| rs1011749 | rs4347628 | 1 | 0.737737 | 1.38E−25 | Chr16 | 88038370 |
| rs2911265 | rs4347628 | 1 | 0.734579 | 5.39E−26 | Chr16 | 88038829 |
| rs2965817 | rs4347628 | 1 | 0.710145 | 1.98E−23 | Chr16 | 88040735 |
| rs17783751 | rs3751688 | 0.890997 | 0.425879 | 8.03E−09 | Chr16 | 88041485 |
| rs2965816 | rs4347628 | 1 | 0.734579 | 5.39E−26 | Chr16 | 88041684 |
| rs2911262 | rs4347628 | 1 | 0.737737 | 3.17E−26 | Chr16 | 88042542 |
| rs9929800 | rs4347628 | 1 | 0.595142 | 1.86E−21 | Chr16 | 88042754 |
| rs2965946 | rs2965946 | 1 | 1 | 0 | Chr16 | 88044113 |
| rs2353581 | rs4347628 | 1 | 0.737737 | 3.17E−26 | Chr16 | 88046409 |
| rs2911244 | rs4347628 | 1 | 0.737737 | 3.17E−26 | Chr16 | 88049361 |
| rs8055457 | rs2965946 | 1 | 0.650485 | 1.29E−22 | Chr16 | 88057588 |
| rs7196903 | rs3751688 | 0.893348 | 0.487973 | 1.54E−09 | Chr16 | 88066260 |
| rs4238830 | rs4347628 | 1 | 0.762787 | 4.61E−27 | Chr16 | 88067742 |
| rs11643561 | rs3751688 | 0.893348 | 0.487973 | 1.54E−09 | Chr16 | 88069627 |
| rs12446145 | rs4347628 | 1 | 0.762787 | 4.61E−27 | Chr16 | 88070196 |
| rs12935112 | rs4347628 | 1 | 0.762787 | 4.61E−27 | Chr16 | 88070359 |
| rs12935119 | rs4347628 | 1 | 0.690141 | 1.14E−24 | Chr16 | 88070375 |
| rs12935033 | rs4347628 | 1 | 0.443859 | 2.73E−16 | Chr16 | 88070689 |
| rs8051537 | rs4347628 | 1 | 0.7569 | 2.81E−26 | Chr16 | 88071885 |
| rs3803682 | rs4347628 | 1 | 0.75988 | 3.50E−26 | Chr16 | 88072137 |
| rs3803681 | rs4347628 | 1 | 0.762787 | 4.61E−27 | Chr16 | 88072257 |
| rs8043788 | rs382745 | 0.963985 | 0.838657 | 2.00E−28 | Chr16 | 88077063 |
| rs8050512 | rs4347628 | 1 | 0.762787 | 4.61E−27 | Chr16 | 88079821 |
| rs4785568 | rs4347628 | 1 | 0.7569 | 1.33E−26 | Chr16 | 88081208 |
| rs4785569 | rs4347628 | 1 | 1 | 1.49E−36 | Chr16 | 88082815 |
| rs4329923 | rs4347628 | 1 | 0.762787 | 4.61E−27 | Chr16 | 88087679 |
| rs4785571 | rs4347628 | 1 | 0.762787 | 4.61E−27 | Chr16 | 88092670 |
| rs4785573 | rs2965946 | 1 | 1 | 1.47E−34 | Chr16 | 88092904 |
| rs9921048 | rs4347628 | 1 | 0.965986 | 1.20E−34 | Chr16 | 88094785 |
| rs11645860 | rs3751688 | 0.893348 | 0.487973 | 1.54E−09 | Chr16 | 88096716 |
| rs4347628 | rs4347628 | 1 | 1 | 0 | Chr16 | 88098136 |
| rs9922341 | rs4347628 | 1 | 1 | 3.56E−37 | Chr16 | 88099316 |
| rs3809641 | rs382745 | 1 | 0.966704 | 2.00E−35 | Chr16 | 88101667 |
| rs3922634 | rs382745 | 1 | 0.966704 | 1.03E−34 | Chr16 | 88104267 |
| rs3922633 | rs382745 | 1 | 0.966704 | 2.36E−34 | Chr16 | 88104547 |
| rs8045263 | rs3751688 | 0.893348 | 0.487973 | 1.54E−09 | Chr16 | 88108185 |
| rs8046182 | rs382745 | 1 | 0.966216 | 3.54E−35 | Chr16 | 88108717 |
| rs8052076 | rs382745 | 1 | 0.965714 | 1.43E−34 | Chr16 | 88111310 |
| rs12919314 | rs3751688 | 0.878253 | 0.42617 | 9.24E−08 | Chr16 | 88113989 |
| rs12924776 | rs3751688 | 0.807863 | 0.526626 | 8.08E−06 | Chr16 | 88114093 |
| rs11862081 | rs382745 | 1 | 0.966216 | 3.54E−35 | Chr16 | 88115989 |
| rs4785687 | rs2965946 | 0.957589 | 0.664341 | 5.05E−21 | Chr16 | 88116397 |
| rs8060502 | rs2965946 | 1 | 0.691451 | 1.56E−23 | Chr16 | 88116909 |
| rs3803680 | rs382745 | 1 | 0.966704 | 1.03E−34 | Chr16 | 88117744 |
| rs3803679 | rs382745 | 1 | 0.966704 | 4.55E−35 | Chr16 | 88118168 |
| rs8051680 | rs3751688 | 0.853447 | 0.45718 | 4.89E−07 | Chr16 | 88118326 |
| rs11643271 | rs3751688 | 0.893197 | 0.483463 | 1.85E−09 | Chr16 | 88119554 |
| rs3935627 | rs382745 | 0.95443 | 0.567592 | 7.19E−19 | Chr16 | 88120123 |
| rs3803677 | rs382745 | 1 | 0.966704 | 2.00E−35 | Chr16 | 88123224 |
| rs3803676 | rs382745 | 1 | 0.966704 | 2.00E−35 | Chr16 | 88123607 |
| rs463701 | rs382745 | 1 | 1 | 7.30E−37 | Chr16 | 88126261 |
| rs2460456 | rs382745 | 0.930796 | 0.837405 | 3.33E−28 | Chr16 | 88129394 |
| rs11640186 | rs382745 | 1 | 0.966704 | 2.00E−35 | Chr16 | 88129507 |
| rs382745 | rs382745 | 1 | 1 | 0 | Chr16 | 88131087 |
| rs2056309 | rs382745 | 1 | 0.900662 | 8.54E−32 | Chr16 | 88134915 |
| rs457372 | rs382745 | 1 | 0.704142 | 1.59E−24 | Chr16 | 88135736 |
| rs462464 | rs3751688 | 1 | 0.504587 | 1.18E−11 | Chr16 | 88140538 |
| rs2292954 | rs3751688 | 1 | 0.571865 | 2.01E−12 | 0hr16 | 88140624 |
| rs461405 | rs3751688 | 1 | 0.473222 | 2.86E−11 | Chr16 | 88140734 |
| rs4325552 | rs3751688 | 1 | 0.65378 | 2.05E−12 | Chr16 | 88142954 |
| rs2019604 | rs3751688 | 0.79523 | 0.525281 | 1.75E−09 | Chr16 | 88143266 |
| rs2377056 | rs3751688 | 1 | 0.65378 | 2.05E−12 | Chr16 | 88143656 |
| rs2889543 | rs3751688 | 1 | 0.606772 | 8.91E−13 | Chr16 | 88143668 |
| rs1864155 | rs3751688 | 0.883979 | 0.516327 | 2.11E−09 | Chr16 | 88143733 |
| rs3794632 | rs3751688 | 1 | 0.609121 | 8.08E−13 | Chr16 | 88144565 |
| rs17775174 | rs3751688 | 1 | 0.609121 | 8.08E−13 | Chr16 | 88145019 |
| rs12960 | rs3751688 | 1 | 0.651568 | 2.91E−13 | Chr16 | 88147829 |
| rs3751691 | rs3751688 | 1 | 0.655963 | 2.39E−13 | Chr16 | 88155882 |
| rs12709088 | rs3751688 | 1 | 0.655963 | 2.39E−13 | Chr16 | 88157713 |
| rs12709089 | rs3751688 | 1 | 0.651568 | 2.91E−13 | Chr16 | 88157812 |
| rs12932337 | rs3751688 | 1 | 0.543726 | 2.68E−10 | Chr16 | 88158131 |
| rs17471624 | rs3751688 | 1 | 0.755269 | 1.59E−10 | Chr16 | 88159166 |
| rs3751688 | rs3751688 | 1 | 1 | 0 | Chr16 | 88161940 |
| rs414998 | rs3751688 | 1 | 0.425608 | 1.07E−10 | Chr16 | 88166727 |
| rs455527 | rs455527 | 1 | 1 | 0 | Chr16 | 88171502 |
| rs352935 | rs352935 | 1 | 1 | 0 | Chr16 | 88176081 |
| rs452176 | rs7188458 | 0.628866 | 0.359734 | 2.21E−10 | Chr16 | 88180533 |
| rs464349 | rs464349 | 1 | 1 | 0 | Chr16 | 88183752 |
| rs464274 | rs464349 | 1 | 1 | 8.47E−38 | Chr16 | 88184132 |
| rs694285 | rs154659 | 0.940421 | 0.527876 | 2.77E−14 | Chr16 | 88190491 |
| rs659974 | rs154659 | 0.938664 | 0.504054 | 8.47E−14 | Chr16 | 88190573 |
| rs154659 | rs154659 | 1 | 1 | 0 | Chr16 | 88194838 |
| rs441526 | rs164741 | 0.772009 | 0.311071 | 1.77E−08 | Chr16 | 88198837 |
| rs8058428 | rs164741 | 0.666442 | 0.280733 | 7.96E−08 | Chr16 | 88199869 |
| rs8059821 | rs460879 | 0.774137 | 0.522969 | 6.58E−15 | Chr16 | 88202569 |
| rs4785698 | rs258322 | 0.857398 | 0.571995 | 2.98E−13 | Chr16 | 88202820 |
| rs16965867 | rs2270460 | 0.678095 | 0.25634 | 0.000039 | Chr16 | 88208369 |
| rs455344 | rs459920 | 0.62198 | 0.242046 | 9.42E−07 | Chr16 | 88209050 |
| rs460105 | rs460879 | 0.858123 | 0.665721 | 4.26E−20 | Chr16 | 88209507 |
| rs154665 | rs164741 | 1 | 0.647218 | 2.26E−23 | Chr16 | 88218189 |
| rs12930346 | rs164741 | 1 | 0.458306 | 9.83E−16 | Chr16 | 88219270 |
| rs164741 | rs164741 | 1 | 1 | 0 | Chr16 | 88219799 |
| rs3794633 | rs258322 | 0.863518 | 0.534567 | 1.04E−12 | Chr16 | 88223939 |
| rs908951 | rs460879 | 0.964799 | 0.869818 | 1.52E−28 | Chr16 | 88225126 |
| rs2070992 | rs258324 | 1 | 0.736119 | 1.40E−15 | Chr16 | 88230121 |
| rs445537 | rs460879 | 0.965117 | 0.868093 | 2.43E−28 | Chr16 | 88230925 |
| rs154657 | rs460879 | 1 | 1 | 1.05E−37 | Chr16 | 88235597 |
| rs164749 | rs460879 | 1 | 1 | 1.05E−37 | Chr16 | 88235725 |
| rs164748 | rs460879 | 1 | 1 | 1.05E−37 | Chr16 | 88235793 |
| rs460879 | rs460879 | 1 | 1 | 0 | Chr16 | 88240390 |
| rs2437956 | rs2965946 | 0.715938 | 0.461979 | 4.90E−13 | Chr16 | 88243752 |
| rs467357 | rs459920 | 1 | 1 | 1.89E−37 | Chr16 | 88244898 |
| rs11647958 | rs7188458 | 1 | 1 | 1.81E−37 | Chr16 | 88248410 |
| rs154663 | rs258324 | 1 | 0.865546 | 1.12E−18 | Chr16 | 88253536 |
| rs7188458 | rs7188458 | 1 | 1 | 0 | Chr16 | 88253985 |
| rs164753 | rs258324 | 0.851393 | 0.454627 | 2.11E−11 | Chr16 | 88255096 |
| rs258330 | rs459920 | 1 | 1 | 8.47E−38 | Chr16 | 88257075 |
| rs459920 | rs459920 | 1 | 1 | 0 | Chr16 | 88258328 |
| rs166297 | rs258324 | 1 | 0.865546 | 9.75E−19 | Chr16 | 88258686 |
| rs258319 | rs459920 | 1 | 1 | 8.47E−38 | Chr16 | 88259525 |
| rs258318 | rs258324 | 1 | 0.865546 | 9.75E−19 | Chr16 | 88259551 |
| rs8062346 | rs258324 | 1 | 0.865546 | 9.75E−19 | Chr16 | 88260169 |
| rs447735 | rs459920 | 0.96632 | 0.933775 | 1.54E−31 | Chr16 | 88261850 |
| rs2377058 | rs7188458 | 1 | 0.45098 | 4.85E−16 | Chr16 | 88262332 |
| rs9937322 | rs7188458 | 1 | 0.471249 | 1.09E−16 | Chr16 | 88263424 |
| rs2434871 | rs258324 | 0.925479 | 0.558456 | 4.02E−13 | Chr16 | 88263853 |
| rs2115401 | rs460879 | 1 | 0.902965 | 2.73E−32 | Chr16 | 88268110 |
| rs12918773 | rs258322 | 1 | 1 | 2.09E−24 | Chr16 | 88268904 |
| rs12443954 | rs258324 | 0.92641 | 0.564296 | 7.40E−14 | Chr16 | 88268997 |
| rs12446791 | rs258324 | 0.916282 | 0.558175 | 3.03E−12 | Chr16 | 88269028 |
| rs12922197 | rs258322 | 1 | 1 | 2.96E−25 | Chr16 | 88272310 |
| rs11645553 | rs2270460 | 0.821637 | 0.571372 | 1.52E−07 | Chr16 | 88275379 |
| rs12924572 | rs7188458 | 0.964172 | 0.898067 | 6.33E−30 | Chr16 | 88276237 |
| rs648548 | rs460879 | 0.928692 | 0.805699 | 7.71E−25 | Chr16 | 88276937 |
| rs3751700 | rs258324 | 1 | 1 | 4.37E−23 | Chr16 | 88279695 |
| rs2277905 | rs258324 | 1 | 0.935275 | 2.16E−21 | Chr16 | 88280002 |
| rs397891 | rs460879 | 0.965531 | 0.900034 | 9.84E−30 | Chr16 | 88280532 |
| rs3794637 | rs258324 | 1 | 1 | 1.61E−19 | Chr16 | 88281071 |
| rs258324 | rs258324 | 1 | 1 | 0 | Chr16 | 88281756 |
| rs17784285 | rs258324 | 1 | 1 | 4.37E−23 | Chr16 | 88281918 |
| rs12924138 | rs7188458 | 0.96584 | 0.931549 | 1.03E−30 | Chr16 | 88281945 |
| rs258322 | rs258322 | 1 | 1 | 0 | Chr16 | 88283404 |
| rs164744 | rs7188458 | 0.965899 | 0.932962 | 1.99E−32 | Chr16 | 88284269 |
| rs12598665 | rs258324 | 1 | 1 | 4.37E−23 | Chr16 | 88284633 |
| rs164742 | rs7188458 | 0.965885 | 0.931984 | 5.90E−32 | Chr16 | 88285226 |
| rs2162943 | rs6500437 | 0.797956 | 0.514284 | 1.38E−14 | Chr16 | 88288153 |
| rs1946482 | rs1946482 | 1 | 1 | 0 | Chr16 | 88289911 |
| rs8404 | rs7196459 | 0.739912 | 0.227317 | 0.002915 | Chr16 | 88290148 |
| rs1045814 | rs6500437 | 0.702767 | 0.421306 | 3.37E−10 | Chr16 | 88290416 |
| rs4247353 | rs6500437 | 0.750506 | 0.453985 | 3.21E−11 | Chr16 | 88290715 |
| rs462769 | rs6500437 | 0.612708 | 0.326015 | 5.95E−09 | Chr16 | 88290764 |
| rs3803690 | rs6500437 | 0.743144 | 0.469002 | 1.27E−11 | Chr16 | 88290860 |
| rs3751696 | rs6500437 | 0.729412 | 0.458131 | 1.18E−12 | Chr16 | 88291120 |
| rs3751695 | rs8058895 | 0.558753 | 0.287694 | 4.67E−07 | Chr16 | 88292050 |
| rs12149952 | rs11861084 | 0.849617 | 0.695742 | 3.01E−20 | Chr16 | 88307591 |
| rs3809646 | rs1800359 | 1 | 0.862188 | 5.85E−29 | Chr16 | 88314356 |
| rs8056353 | rs8058895 | 1 | 1 | 5.23E−29 | Chr16 | 88316812 |
| rs6500437 | rs6500437 | 1 | 1 | 0 | Chr16 | 88317399 |
| rs4785590 | rs1800359 | 1 | 0.962963 | 2.96E−32 | Chr16 | 88317668 |
| rs8048331 | rs11861084 | 1 | 0.962963 | 2.48E−32 | Chr16 | 88320543 |
| rs2099105 | rs8058895 | 1 | 0.790298 | 2.54E−22 | Chr16 | 88321056 |
| rs13339414 | rs1946482 | 0.806302 | 0.465719 | 5.54E−09 | Chr16 | 88322882 |
| rs7204478 | rs7204478 | 1 | 1 | 0 | Chr16 | 88322986 |
| rs11640450 | rs2270460 | 1 | 0.698113 | 9.11E−09 | Chr16 | 88323360 |
| rs7185737 | rs16966142 | 1 | 1 | 1.05E−17 | Chr16 | 88323511 |
| rs4785709 | rs1800359 | 1 | 0.956394 | 1.99E−29 | Chr16 | 88324166 |
| rs4785710 | rs11861084 | 1 | 0.927733 | 2.76E−31 | Chr16 | 88324257 |
| rs12709092 | rs2270460 | 0.687093 | 0.362696 | 5.09E−06 | Chr16 | 88324838 |
| rs4785713 | rs6500437 | 0.962885 | 0.864158 | 4.50E−27 | Chr16 | 88325599 |
| rs4785714 | rs11861084 | 1 | 0.927733 | 2.76E−31 | Chr16 | 88325905 |
| rs4785594 | rs8058895 | 1 | 0.697161 | 7.02E−21 | Chr16 | 88326017 |
| rs17177891 | rs2270460 | 1 | 0.479799 | 9.36E−06 | Chr16 | 88327451 |
| rs3803689 | rs2239359 | 0.890122 | 0.7653 | 4.54E−23 | Chr16 | 88327559 |
| rs9935559 | rs1800359 | 1 | 0.96084 | 2.70E−29 | Chr16 | 88328030 |
| rs7189734 | rs8058895 | 1 | 0.82615 | 3.23E−21 | Chr16 | 88328051 |
| rs11649155 | rs8058895 | 1 | 0.792244 | 519E−23 | Chr16 | 88329393 |
| rs11649196 | rs1946482 | 0.546059 | 0.234706 | 0.000036 | Chr16 | 88329453 |
| rs7202427 | rs8058895 | 1 | 0.792244 | 5.19E−23 | Chr16 | 88329897 |
| rs1230 | rs6500437 | 0.963235 | 0.808869 | 2.60E−26 | Chr16 | 88332356 |
| rs1800359 | rs1800359 | 1 | 1 | 0 | Chr16 | 88332762 |
| rs9282681 | rs2270460 | 0.820302 | 0.570424 | 1.59E−07 | Chr16 | 88333415 |
| rs1061646 | rs6500437 | 1 | 1 | 5.26E−37 | Chr16 | 88333478 |
| rs7195906 | rs6500437 | 1 | 0.842767 | 9.17E−30 | Chr16 | 88333848 |
| rs11644967 | rs2270460 | 1 | 0.698113 | 9.94E−09 | Chr16 | 88334115 |
| rs11648689 | rs2270460 | 1 | 0.311352 | 0.003786 | Chr16 | 88334233 |
| rs11649162 | rs2270460 | 1 | 0.698113 | 9.11E−09 | Chr16 | 88334632 |
| rs11649210 | rs2270460 | 1 | 0.698113 | 9.11E−09 | Chr16 | 88334734 |
| rs11640188 | rs2270460 | 1 | 0.698113 | 9.11E−09 | Chr16 | 88335233 |
| rs11640209 | rs2270460 | 1 | 0.698113 | 9.52E−09 | Chr16 | 88335329 |
| rs6500439 | rs6500437 | 1 | 0.839706 | 3.01E−29 | Chr16 | 88335776 |
| rs6500440 | rs1800359 | 1 | 1 | 2.42E−29 | Chr16 | 88338324 |
| rs12917681 | rs2270460 | 1 | 0.698113 | 9.11E−09 | Chr16 | 88338442 |
| rs2074904 | rs2270460 | 1 | 0.698113 | 9.52E−09 | Chr16 | 88339047 |
| rs2074903 | rs2270460 | 1 | 0.698113 | 9.11E−09 | Chr16 | 88339164 |
| rs12922302 | rs6500437 | 0.96284 | 0.807504 | 5.07E−26 | Chr16 | 88339784 |
| rs12102290 | rs6500437 | 0.963235 | 0.808869 | 2.60E−26 | Chr16 | 88340118 |
| rs12102297 | rs6500437 | 0.962416 | 0.789609 | 1.03E−23 | Chr16 | 88340263 |
| rs1800355 | rs2270460 | 1 | 0.698113 | 9.11E−09 | Chr16 | 88340695 |
| rs11641147 | rs2270460 | 1 | 0.311352 | 0.003786 | Chr16 | 88341090 |
| rs4420527 | rs6500437 | 1 | 0.840764 | 1.53E−29 | Chr16 | 88341689 |
| rs8058895 | rs8058895 | 1 | 1 | 0 | Chr16 | 88342308 |
| rs2011877 | rs2011877 | 1 | 1 | 0 | Chr16 | 88342319 |
| rs12599002 | rs2270460 | 0.822953 | 0.572307 | 1.45E−07 | Chr16 | 88345070 |
| rs3743860 | rs460879 | 0.852256 | 0.608527 | 5.60E−18 | Chr16 | 88345992 |
| rs11649642 | rs8058895 | 1 | 0.790298 | 6.70E−23 | Chr16 | 88347433 |
| rs7195752 | rs6500437 | 1 | 1 | 5.26E−37 | Chr16 | 88349461 |
| rs7201028 | rs6500437 | 1 | 1 | 5.26E−37 | Chr16 | 88349619 |
| rs2238526 | rs8060934 | 0.925324 | 0.795428 | 1.11E−23 | Chr16 | 88354224 |
| rs2239357 | rs2270460 | 1 | 0.477274 | 0.000103 | Chr16 | 88354752 |
| rs2239358 | rs2270460 | 1 | 0.648456 | 1.74E−07 | Chr16 | 88354831 |
| rs11076619 | rs2270460 | 1 | 0.648456 | 1.89E−07 | Chr16 | 88358744 |
| rs2159116 | rs8058895 | 1 | 1 | 5.15E−29 | Chr16 | 88359011 |
| rs12600151 | rs2270460 | 1 | 0.477274 | 0.000099 | Chr16 | 88359059 |
| rs2159114 | rs1800359 | 1 | 0.9273 | 6.09E−31 | Chr16 | 88359342 |
| rs2159113 | rs6500437 | 1 | 0.932432 | 5.50E−33 | Chr16 | 88359584 |
| rs7203907 | rs6500437 | 0.961556 | 0.782192 | 9.42E−23 | Chr16 | 88361275 |
| rs11860203 | rs6500437 | 1 | 0.926489 | 7.33E−30 | Chr16 | 88362162 |
| rs11645916 | rs2270460 | 1 | 0.379824 | 0.000228 | Chr16 | 88362735 |
| rs3890534 | rs16966142 | 1 | 1 | 1.02E−18 | Chr16 | 88362790 |
| rs4785595 | rs6500437 | 0.927298 | 0.77558 | 2.26E−24 | Chr16 | 88363022 |
| rs7195066 | rs7195066 | 1 | 1 | 0 | Chr16 | 88363824 |
| rs886952 | rs6500437 | 0.927416 | 0.775778 | 1.13E−24 | Chr16 | 88364282 |
| rs886950 | rs6500437 | 0.962361 | 0.806058 | 4.55E−25 | Chr16 | 88364373 |
| rs11644213 | rs2270460 | 1 | 0.648456 | 1.74E−07 | Chr16 | 88364868 |
| rs1007932 | rs2270460 | 1 | 0.698113 | 9.11E−09 | Chr16 | 88366042 |
| rs7190403 | rs8058895 | 1 | 1 | 2.32E−28 | Chr16 | 88366186 |
| rs12599180 | rs6500437 | 0.927416 | 0.775778 | 1.13E−24 | Chr16 | 88366807 |
| rs1800339 | rs2270460 | 1 | 0.847328 | 1.12E−10 | Chr16 | 88367138 |
| rs2238527 | rs2270460 | 1 | 0.698113 | 9.11E−09 | Chr16 | 88368209 |
| rs8046872 | rs1800359 | 1 | 0.926874 | 3.56E−30 | Chr16 | 88369053 |
| rs12596934 | rs2270460 | 1 | 0.821788 | 1.98E−09 | Chr16 | 88369106 |
| rs3785275 | rs6500437 | 1 | 0.965753 | 1.07E−34 | Chr16 | 88369530 |
| rs1006548 | rs7195066 | 1 | 0.808586 | 1.54E−24 | Chr16 | 88371544 |
| rs11076623 | rs2270460 | 1 | 0.698113 | 9.11E−09 | Chr16 | 88371621 |
| rs1006547 | rs6500437 | 1 | 0.965035 | 4.45E−34 | Chr16 | 88371730 |
| rs2016571 | rs6500437 | 1 | 0.965753 | 1.07E−34 | Chr16 | 88371777 |
| rs7187436 | rs6500437 | 0.963707 | 0.927202 | 7.64E−29 | Chr16 | 88372611 |
| rs1800337 | rs6500437 | 0.927416 | 0.775778 | 1.13E−24 | Chr16 | 88372695 |
| rs3743859 | rs6500437 | 0.927416 | 0.775778 | 1.13E−24 | Chr16 | 88373551 |
| rs1800335 | rs6500437 | 0.925701 | 0.772182 | 1.98E−23 | Chr16 | 88373696 |
| rs8058179 | rs1800359 | 1 | 0.89418 | 4.71E−30 | Chr16 | 88374488 |
| rs2239359 | rs2239359 | 1 | 1 | 0 | Chr16 | 88376981 |
| rs2239360 | rs6500437 | 1 | 0.931034 | 2.25E−32 | Chr16 | 88377084 |
| rs12448860 | rs6500437 | 0.927416 | 0.775778 | 1.13E−24 | Chr16 | 88377130 |
| rs11649501 | rs2270460 | 1 | 0.698113 | 9.11E−09 | Chr16 | 88378458 |
| rs16966142 | rs16966142 | 1 | 1 | 0 | Chr16 | 88378534 |
| rs8046243 | rs6500437 | 0.925904 | 0.774379 | 6.07E−24 | Chr16 | 88379634 |
| rs12709094 | rs6500437 | 0.963075 | 0.800575 | 1.94E−25 | Chr16 | 88380518 |
| rs2238529 | rs6500437 | 1 | 0.92126 | 2.48E−29 | Chr16 | 88380618 |
| rs2238531 | rs2270460 | 1 | 0.698113 | 9.52E−09 | Chr16 | 88383718 |
| rs6500449 | rs6500437 | 0.92722 | 0.773588 | 3.67E−24 | Chr16 | 88383894 |
| rs17746039 | rs8058895 | 1 | 1 | 5.15E−29 | Chr16 | 88383982 |
| rs8045232 | rs6500437 | 0.925587 | 0.771987 | 1.67E−23 | Chr16 | 88385049 |
| rs1057042 | rs6500437 | 1 | 0.965753 | 1.07E−34 | Chr16 | 88385123 |
| rs8049660 | rs6500437 | 1 | 0.932432 | 2.70E−31 | Chr16 | 88385201 |
| rs11646374 | rs2270460 | 1 | 0.698113 | 9.11E−09 | Chr16 | 88385436 |
| rs1800330 | rs6500437 | 1 | 0.932432 | 2.37E−32 | Chr16 | 88385465 |
| rs6500450 | rs6500437 | 0.922226 | 0.664304 | 3.70E−20 | Chr16 | 88385525 |
| rs1800331 | rs2270460 | 1 | 0.698113 | 9.11E−09 | Chr16 | 88385918 |
| rs6500452 | rs6500437 | 1 | 0.931034 | 2.25E−32 | Chr16 | 88386006 |
| rs1800287 | rs6500437 | 0.926463 | 0.757848 | 2.28E−22 | Chr16 | 88386026 |
| rs6500453 | rs6500437 | 0.961515 | 0.79335 | 1.89E−23 | Chr16 | 88386158 |
| rs12921383 | rs258322 | 1 | 0.868421 | 2.96E−19 | Chr16 | 88387254 |
| rs8051231 | rs6500437 | 1 | 0.931034 | 1.38E−32 | Chr16 | 88389633 |
| rs11648881 | rs2270460 | 1 | 0.698113 | 9.11E−09 | Chr16 | 88389935 |
| rs12924101 | rs258322 | 0.664822 | 0.3771 | 2.80E−08 | Chr16 | 88390407 |
| rs12709096 | rs6500437 | 1 | 0.932432 | 8.94E−33 | Chr16 | 88390462 |
| rs4785722 | rs6500437 | 1 | 0.932432 | 5.50E−33 | Chr16 | 88390611 |
| rs11076626 | rs6500437 | 0.927416 | 0.775778 | 1.13E−24 | Chr16 | 88392604 |
| rs10852623 | rs6500437 | 0.963137 | 0.806775 | 8.61E−26 | Chr16 | 88392743 |
| rs7190823 | rs6500437 | 0.963196 | 0.806875 | 4.29E−26 | Chr16 | 88393544 |
| rs12599799 | rs6500437 | 0.927416 | 0.775778 | 1.13E−24 | Chr16 | 88394869 |
| rs1800286 | rs11861084 | 1 | 1 | 2.19E−35 | Chr16 | 88397262 |
| rs11076628 | rs6500437 | 1 | 0.8125 | 3.40E−28 | Chr16 | 88402747 |
| rs11861084 | rs11861084 | 1 | 1 | 0 | Chr16 | 88403211 |
| rs2074963 | rs2270460 | 1 | 0.698113 | 9.52E−09 | Chr16 | 88404770 |
| rs11076631 | rs6500437 | 1 | 0.814815 | 9.09E−29 | Chr16 | 88405476 |
| rs11076632 | rs6500437 | 1 | 0.814815 | 9.09E−29 | Chr16 | 88408038 |
| rs2079672 | rs11861084 | 0.962928 | 0.92723 | 7.31E−29 | Chr16 | 88412418 |
| rs1108064 | rs11861084 | 1 | 0.896433 | 1.35E−30 | Chr16 | 88421651 |
| rs11641891 | rs11861084 | 1 | 0.773585 | 6.78E−26 | Chr16 | 88424650 |
| rs11644526 | rs2270460 | 1 | 0.477277 | 0.000103 | Chr16 | 88426161 |
| rs6500457 | rs11861084 | 1 | 0.773585 | 6.78E−26 | Chr16 | 88426265 |
| rs4785727 | rs8060934 | 1 | 0.867367 | 6.04E−30 | Chr16 | 88427738 |
| rs9928396 | rs8060934 | 1 | 0.867367 | 6.04E−30 | Chr16 | 88433349 |
| rs9935541 | rs11861084 | 1 | 0.962676 | 9.77E−31 | Chr16 | 88433426 |
| rs1079558 | rs6500437 | 0.891706 | 0.766746 | 7.67E−23 | Chr16 | 88435504 |
| rs12599531 | rs11861084 | 1 | 0.858948 | 2.25E−28 | Chr16 | 88436902 |
| rs17177912 | rs885479 | 1 | 0.891892 | 3.50E−14 | Chr16 | 88436930 |
| rs17719345 | rs8058895 | 0.894221 | 0.693497 | 4.52E−17 | Chr16 | 88439182 |
| rs10775347 | rs6500437 | 0.889266 | 0.687106 | 3.25E−21 | Chr16 | 88442009 |
| rs10852626 | rs11861084 | 1 | 0.858948 | 3.27E−28 | Chr16 | 88442134 |
| rs8060934 | rs8060934 | 1 | 1 | 0 | Chr16 | 88447526 |
| rs7200842 | rs8060934 | 1 | 1 | 6.30E−36 | Chr16 | 88453057 |
| rs12446215 | rs3803688 | 1 | 1 | 2.86E−21 | Chr16 | 88456355 |
| rs3803688 | rs3803688 | 1 | 1 | 0 | Chr16 | 88462387 |
| rs1109334 | rs8060934 | 0.924444 | 0.767737 | 3.67E−23 | Chr16 | 88465645 |
| rs11640336 | rs2270460 | 1 | 1 | 3.13E−13 | Chr16 | 88468654 |
| rs12919804 | rs2270460 | 1 | 1 | 7.55E−07 | Chr16 | 88472870 |
| rs7192387 | rs3212346 | 1 | 0.20904 | 0.00101 | Chr16 | 88473892 |
| rs12930056 | rs2270460 | 1 | 1 | 3.33E−13 | Chr16 | 88474150 |
| rs12930606 | rs2270460 | 1 | 1 | 3.33E−13 | Chr16 | 88474183 |
| rs17784386 | rs2270460 | 1 | 1 | 3.13E−13 | Chr16 | 88474958 |
| rs9806913 | rs3212346 | 1 | 1 | 1.29E−15 | Chr16 | 88481274 |
| rs9922515 | rs3212346 | 1 | 1 | 1.51E−15 | Chr16 | 88481639 |
| rs11643448 | rs2270460 | 1 | 1 | 3.13E−13 | Chr16 | 88483748 |
| rs11639625 | rs2270460 | 1 | 1 | 3.13E−13 | Chr16 | 88483754 |
| rs4287569 | rs3212346 | 1 | 1 | 1.29E−15 | Chr16 | 88485316 |
| rs7191836 | rs3212346 | 1 | 0.880952 | 4.94E−13 | Chr16 | 88489098 |
| rs7184960 | rs3212346 | 1 | 0.880952 | 4.29E−13 | Chr16 | 88489162 |
| rs11641790 | rs3212346 | 1 | 1 | 1.29E−15 | Chr16 | 88489458 |
| rs10153055 | rs3212346 | 1 | 1 | 1.29E−15 | Chr16 | 88493343 |
| rs11646448 | rs2270460 | 1 | 1 | 3.13E−13 | Chr16 | 88493858 |
| rs10153210 | rs3212346 | 1 | 0.20904 | 0.00101 | Chr16 | 88494809 |
| rs10153196 | rs3212346 | 1 | 1 | 1.79E−14 | Chr16 | 88494898 |
| rs2270461 | rs3212346 | 1 | 1 | 1.29E−15 | Chr16 | 88499846 |
| rs2270460 | rs2270460 | 1 | 1 | 0 | Chr16 | 88499917 |
| rs8045560 | rs8045560 | 1 | 1 | 0 | Chr16 | 88506995 |
| rs2270459 | rs2270460 | 1 | 1 | 3.13E−13 | Chr16 | 88507352 |
| rs3212345 | rs8045560 | 1 | 1 | 4.31E−37 | Chr16 | 88509773 |
| rs3212346 | rs3212346 | 1 | 1 | 0 | Chr16 | 88509859 |
| rs3212363 | rs8045560 | 1 | 0.608665 | 2.22E−21 | Chr16 | 88512942 |
| rs1805005 | rs1946482 | 0.806302 | 0.465719 | 5.54E−09 | Chr16 | 88513345 |
| rs1805007 | rs258322 | 0.931248 | 0.712538 | 4.20E−16 | Chr16 | 88513618 |
| rs885479 | rs885479 | 1 | 1 | 0 | Chr16 | 88513655 |
| rs7191944 | rs8045560 | 0.690581 | 0.335339 | 1.92E−09 | Chr16 | 88521379 |
| rs2302898 | rs8045560 | 0.65735 | 0.305049 | 8.49E−09 | Chr16 | 88526295 |
| rs4395073 | rs8045560 | 0.667049 | 0.388196 | 1.93E−11 | Chr16 | 88530183 |
| rs4558416 | rs8045560 | 0.849473 | 0.608982 | 3.11E−18 | Chr16 | 88530268 |
| rs4785741 | rs8045560 | 0.668391 | 0.402087 | 1.14E−11 | Chr16 | 88532954 |
| rs4785742 | rs8045560 | 0.716361 | 0.412832 | 3.51E−11 | Chr16 | 88533062 |
| rs7184956 | rs8045560 | 0.687898 | 0.467482 | 6.59E−13 | Chr16 | 88534535 |
| rs11644157 | rs2270460 | 0.848269 | 0.717599 | 2.00E−09 | Chr16 | 88535797 |
| rs12598666 | rs2270460 | 1 | 0.847328 | 1.07E−10 | Chr16 | 88537213 |
| rs13338472 | rs2270460 | 1 | 0.847328 | 1.54E−10 | Chr16 | 88538756 |
| rs4365287 | rs2270460 | 1 | 0.847328 | 1.07E−10 | Chr16 | 88541458 |
| rs6500462 | rs7196459 | 0.799833 | 0.639733 | 5.09E−11 | Chr16 | 88543461 |
| rs7195043 | rs4408545 | 0.726485 | 0.351413 | 4.50E−10 | Chr16 | 88548362 |
| rs12597913 | rs2270460 | 1 | 0.698113 | 9.52E−09 | Chr16 | 88549186 |
| rs12596206 | rs2270460 | 1 | 0.698113 | 9.11E−09 | Chr16 | 88550239 |
| rs11641639 | rs2270460 | 1 | 0.698113 | 9.11E−09 | Chr16 | 88550906 |
| rs8049897 | rs258322 | 0.737677 | 0.491313 | 9.45E−11 | Chr16 | 88551703 |
| rs8051733 | rs4238833 | 1 | 0.754676 | 3.63E−23 | Chr16 | 88551707 |
| rs7187431 | rs2270460 | 1 | 0.697523 | 1.14E−08 | Chr16 | 88552401 |
| rs17784583 | rs885479 | 0.869119 | 0.371079 | 1.35E−07 | Chr16 | 88554480 |
| rs8063761 | rs4785763 | 0.920972 | 0.730534 | 6.69E−23 | Chr16 | 88555127 |
| rs8062311 | rs9936896 | 1 | 0.603322 | 1.53E−13 | Chr16 | 88555339 |
| rs8048449 | rs9936896 | 0.921965 | 0.583409 | 2.78E−13 | Chr16 | 88555607 |
| rs4785751 | rs4408545 | 0.864105 | 0.722182 | 3.59E−23 | Chr16 | 88556918 |
| rs4785755 | rs4785755 | 1 | 1 | 0 | Chr16 | 88565329 |
| rs4408545 | rs4408545 | 1 | 1 | 0 | Chr16 | 88571529 |
| rs3803684 | rs3212346 | 1 | 0.649123 | 1.79E−09 | Chr16 | 88573874 |
| rs4238833 | rs4238833 | 1 | 1 | 0 | Chr16 | 88578190 |
| rs4785759 | rs4238833 | 1 | 0.782101 | 1.76E−26 | Chr16 | 88578381 |
| rs11643288 | rs2270460 | 1 | 0.698113 | 9.11E−09 | Chr16 | 88580094 |
| rs1004047 | rs2270460 | 1 | 0.552239 | 4.71E−07 | Chr16 | 88580837 |
| rs7201721 | rs7201721 | 1 | 1 | 0 | Chr16 | 88586247 |
| rs11076649 | rs258322 | 0.760403 | 0.575704 | 5.51E−13 | Chr16 | 88586837 |
| rs3803683 | rs258322 | 0.754659 | 0.542641 | 1.83E−12 | Chr16 | 88587782 |
| rs4785763 | rs4785763 | 1 | 1 | 0 | Chr16 | 88594437 |
| rs11076650 | rs4785763 | 1 | 0.704433 | 4.21E−24 | Chr16 | 88595442 |
| rs9936896 | rs9936896 | 1 | 1 | 0 | Chr16 | 88596560 |
| rs11076653 | rs2270460 | 1 | 0.698113 | 1.04E−08 | Chr16 | 88601502 |
| rs11076654 | rs2270460 | 1 | 0.698113 | 9.11E−09 | Chr16 | 88601586 |
| rs2241084 | rs8059973 | 1 | 0.928571 | 8.44E−20 | Chr16 | 88602913 |
| rs8059973 | rs8059973 | 1 | 1 | 0 | Chr16 | 88607035 |
| rs9936215 | rs9936215 | 1 | 1 | 0 | Chr16 | 88609161 |
| rs8057672 | rs11648785 | 1 | 0.963834 | 2.55E−33 | Chr16 | 88610532 |
| rs11648785 | rs11648785 | 1 | 1 | 0 | Chr16 | 88612062 |
| rs2241039 | rs2241039 | 1 | 1 | 0 | Chr16 | 88615938 |
| rs872010 | rs3785181 | 1 | 1 | 1.29E−15 | Chr16 | 88616288 |
| rs870856 | rs11648785 | 1 | 0.858948 | 3.27E−28 | Chr16 | 88616964 |
| rs3743829 | rs2241039 | 1 | 0.809876 | 4.01E−28 | Chr16 | 88621470 |
| rs3743827 | rs2241039 | 1 | 0.964169 | 1.26E−33 | Chr16 | 88621696 |
| rs3743826 | rs11648785 | 1 | 0.927733 | 2.76E−31 | Chr16 | 88621715 |
| rs2302513 | rs3785181 | 1 | 1 | 1.39E−15 | Chr16 | 88625249 |
| rs10431948 | rs2241039 | 1 | 1 | 2.77E−36 | Chr16 | 88627072 |
| rs2241037 | rs2270460 | 1 | 0.698113 | 9.11E−09 | Chr16 | 88629328 |
| rs4785766 | rs4785766 | 1 | 1 | 0 | Chr16 | 88629885 |
| rs868045 | rs7498985 | 1 | 0.8394 | 1.96E−29 | Chr16 | 88630336 |
| rs7498985 | rs7498985 | 1 | 1 | 0 | Chr16 | 88630618 |
| rs4374173 | rs7498985 | 1 | 1 | 6.05E−38 | Chr16 | 88630747 |
| rs17178299 | rs2270460 | 1 | 0.698113 | 9.52E−09 | Chr16 | 88631160 |
| rs2241036 | rs11648785 | 0.957772 | 0.887514 | 3.54E−26 | Chr16 | 88631520 |
| rs11648422 | rs7498985 | 1 | 1 | 1.13E−35 | Chr16 | 88632505 |
| rs3785181 | rs3785181 | 1 | 1 | 0 | Chr16 | 88632834 |
| rs9928084 | rs7498985 | 1 | 0.84472 | 4.21E−30 | Chr16 | 88633227 |
| rs2241032 | rs2241032 | 1 | 1 | 0 | Chr16 | 88637020 |
| rs3743824 | rs8045560 | 0.944331 | 0.438954 | 1.39E−13 | Chr16 | 88637528 |
| rs1048148 | rs8045560 | 0.944331 | 0.438954 | 1.39E−13 | Chr16 | 88637790 |
| rs3743817 | rs2241032 | 1 | 1 | 1.42E−21 | Chr16 | 88638238 |
| rs4628973 | rs2078478 | 0.869717 | 0.460647 | 4.13E−12 | Chr16 | 88639727 |
| rs869048 | rs2078478 | 0.86658 | 0.431211 | 2.57E−11 | Chr16 | 88640449 |
| rs4785612 | rs4785612 | 1 | 1 | 0 | Chr16 | 88640608 |
| rs9921920 | rs2239359 | 0.751638 | 0.344735 | 4.41E−10 | Chr16 | 88643119 |
| rs11639655 | rs2078478 | 1 | 0.457735 | 1.55E−14 | Chr16 | 88643232 |
| rs11642999 | rs2270460 | 0.822953 | 0.572307 | 1.45E−07 | Chr16 | 88645620 |
| rs11642131 | rs2270460 | 1 | 0.480978 | 0.000118 | Chr16 | 88647858 |
| rs11076663 | rs2270460 | 1 | 0.698113 | 9.52E−09 | Chr16 | 88648632 |
| rs8046635 | rs7196459 | 0.874608 | 0.616673 | 3.72E−10 | Chr16 | 88650994 |
| rs3809643 | rs2241032 | 0.916906 | 0.424978 | 1.01E−10 | Chr16 | 88651774 |
| rs3826201 | rs2241032 | 0.927885 | 0.86097 | 7.44E−18 | Chr16 | 88651817 |
| rs7206111 | rs2270460 | 1 | 0.698113 | 9.11E−09 | Chr16 | 88652372 |
| rs9935461 | rs2270460 | 1 | 0.698113 | 9.11E−09 | Chr16 | 88653232 |
| rs9927964 | rs2270460 | 1 | 0.698113 | 9.11E−09 | Chr16 | 88653448 |
| rs11647734 | rs2270460 | 1 | 0.698113 | 9.11E−09 | Chr16 | 88653901 |
| rs3826200 | rs2241032 | 0.927885 | 0.86097 | 7.44E−18 | Chr16 | 88654581 |
| rs2077426 | rs2078478 | 1 | 0.440559 | 3.45E−14 | Chr16 | 88655725 |
| rs4493039 | rs2078478 | 1 | 1 | 5.10E−26 | Chr16 | 88657471 |
| rs2078478 | rs2078478 | 1 | 1 | 0 | Chr16 | 88657637 |
| rs4785621 | rs7196459 | 0.899917 | 0.80985 | 9.56E−14 | Chr16 | 88658236 |
| rs11642823 | rs2270460 | 1 | 0.698113 | 9.11E−09 | Chr16 | 88660287 |
| rs4785625 | rs2078478 | 1 | 0.475839 | 6.72E−15 | Chr16 | 88666345 |
| rs12925933 | rs2078478 | 1 | 0.475839 | 6.72E−15 | Chr16 | 88668856 |
| rs7196459 | rs7196459 | 1 | 1 | 0 | Chr16 | 88668978 |
| rs4785780 | rs2241032 | 0.899093 | 0.336835 | 3.18E−08 | Chr16 | 88670344 |
| rs7187797 | rs3785181 | 1 | 0.6139 | 1.39E−11 | Chr16 | 88671597 |
| rs11642964 | rs2270460 | 1 | 0.698113 | 9.94E−09 | Chr16 | 88673157 |
| rs4785781 | rs7196459 | 0.899917 | 0.80985 | 9.56E−14 | Chr16 | 88676480 |
| rs11643796 | rs7196459 | 0.899917 | 0.80985 | 9.56E−14 | Chr16 | 88677234 |
| rs6500465 | rs2078478 | 0.935477 | 0.77659 | 1.08E−17 | Chr16 | 88680668 |
| rs8047319 | rs11648785 | 0.589908 | 0.233552 | 1.73E−06 | Chr16 | 88684276 |
| rs9922277 | rs9936215 | 0.956829 | 0.750305 | 4.58E−21 | Chr16 | 88686339 |
| rs7498369 | rs9936215 | 0.852254 | 0.676475 | 1.18E−20 | Chr16 | 88687565 |
| rs6500468 | rs9936215 | 0.741168 | 0.44803 | 8.89E−12 | Chr16 | 88688793 |
| rs3889353 | rs3785181 | 1 | 0.888268 | 6.01E−13 | Chr16 | 88690234 |
| rs6500472 | rs7498985 | 0.882346 | 0.593169 | 2.97E−19 | Chr16 | 88691089 |
| rs4545892 | rs4453582 | 0.902484 | 0.292376 | 2.85E−07 | Chr18 | 34622891 |
| rs4476249 | rs4453582 | 0.902484 | 0.292376 | 2.85E−07 | Chr18 | 34623804 |
| rs12604555 | rs4453582 | 0.902484 | 0.292376 | 2.85E−07 | Chr18 | 34624070 |
| rs17574888 | rs4453582 | 0.902484 | 0.292376 | 2.85E−07 | Chr18 | 34630755 |
| rs3961799 | rs4453582 | 1 | 0.350649 | 8.62E−14 | Chr18 | 34630933 |
| rs9304195 | rs4453582 | 0.903909 | 0.299039 | 2.39E−07 | Chr18 | 34632149 |
| rs8098442 | rs4453582 | 1 | 0.458128 | 1.51E−16 | Chr18 | 34637398 |
| rs12606593 | rs4453582 | 0.90064 | 0.291183 | 5.73E−07 | Chr18 | 34637972 |
| rs4570937 | rs4453582 | 0.902484 | 0.292376 | 2.85E−07 | Chr18 | 34638022 |
| rs4570938 | rs4453582 | 0.902484 | 0.292376 | 2.85E−07 | Chr18 | 34638265 |
| rs4476248 | rs4453582 | 1 | 0.458128 | 7.21E−17 | Chr18 | 34638350 |
| rs7505650 | rs4453582 | 0.902484 | 0.292376 | 2.85E−07 | Chr18 | 34639712 |
| rs4799492 | rs4453582 | 0.902484 | 0.292376 | 2.85E−07 | Chr18 | 34644106 |
| rs7230537 | rs4453582 | 0.902484 | 0.292376 | 2.85E−07 | Chr18 | 34647067 |
| rs12962677 | rs4453582 | 1 | 0.350649 | 8.62E−14 | Chr18 | 34648187 |
| rs12608258 | rs4453582 | 0.910484 | 0.325699 | 4.73E−08 | Chr18 | 34653046 |
| rs6507283 | rs4453582 | 1 | 0.371493 | 4.25E−13 | Chr18 | 34653521 |
| rs12606820 | rs4453582 | 0.90912 | 0.318295 | 5.76E−08 | Chr18 | 34656116 |
| rs4800046 | rs4453582 | 0.90912 | 0.318295 | 5.76E−08 | Chr18 | 34656974 |
| rs12604198 | rs4453582 | 0.90912 | 0.318295 | 5.76E−08 | Chr18 | 34662416 |
| rs12604200 | rs4453582 | 0.90912 | 0.318295 | 5.76E−08 | Chr18 | 34662451 |
| rs12607416 | rs4453582 | 0.90912 | 0.318295 | 5.76E−08 | Chr18 | 34664159 |
| rs4800047 | rs4453582 | 0.90912 | 0.318295 | 5.76E−08 | Chr18 | 34664272 |
| rs4800049 | rs4453582 | 0.90912 | 0.318295 | 5.76E−08 | Chr18 | 34665603 |
| rs4800050 | rs4453582 | 0.90912 | 0.318295 | 5.76E−08 | Chr18 | 34665856 |
| rs17653342 | rs4453582 | 0.90912 | 0.318295 | 5.76E−08 | Chr18 | 34666102 |
| rs12607945 | rs4453582 | 0.90912 | 0.318295 | 5.76E−08 | Chr18 | 34668162 |
| rs12604846 | rs4453582 | 0.90912 | 0.318295 | 5.76E−08 | Chr18 | 34670031 |
| rs12607133 | rs4453582 | 0.90912 | 0.318295 | 5.76E−08 | Chr18 | 34670220 |
| rs17575270 | rs4453582 | 0.903185 | 0.302413 | 2.13E−07 | Chr18 | 34676396 |
| rs10502708 | rs4453582 | 0.90912 | 0.318295 | 5.76E−08 | Chr18 | 34676501 |
| rs12968829 | rs4453582 | 1 | 0.259259 | 1.56E−08 | Chr18 | 34677416 |
| rs12969426 | rs4453582 | 1 | 0.259259 | 1.40E−08 | Chr18 | 34677807 |
| rs6507284 | rs4453582 | 1 | 0.458128 | 7.21E−17 | Chr18 | 34681478 |
| rs4799495 | rs4453582 | 1 | 0.350649 | 8.62E−14 | Chr18 | 34681679 |
| rs12607554 | rs4453582 | 0.90912 | 0.318295 | 5.76E−08 | Chr18 | 34683375 |
| rs11082111 | rs4453582 | 1 | 0.375 | 1.46E−13 | Chr18 | 34700583 |
| rs8092910 | rs4453582 | 1 | 0.350649 | 8.62E−14 | Chr18 | 34701021 |
| rs4800056 | rs4453582 | 1 | 0.350649 | 8.62E−14 | Chr18 | 34712479 |
| rs4800057 | rs4453582 | 1 | 0.358407 | 1.02E−13 | Chr18 | 34713860 |
| rs9953997 | rs4453582 | 1 | 0.350649 | 8.62E−14 | Chr18 | 34714302 |
| rs17576167 | rs4453582 | 1 | 0.583333 | 8.55E−18 | Chr18 | 34717205 |
| rs11082112 | rs4453582 | 1 | 0.362637 | 3.83E−14 | Chr18 | 34717239 |
| rs4584902 | rs4453582 | 1 | 0.358407 | 5.51E−14 | Chr18 | 34718756 |
| rs16971051 | rs4453582 | 1 | 0.370787 | 2.40E−14 | Chr18 | 34719563 |
| rs4800058 | rs4453582 | 1 | 1 | 9.56E−34 | Chr18 | 34720502 |
| rs12457299 | rs4453582 | 1 | 0.362637 | 3.83E−14 | Chr18 | 34721810 |
| rs12608331 | rs4453582 | 1 | 1 | 9.56E−34 | Chr18 | 34724913 |
| rs4800059 | rs4453582 | 1 | 1 | 9.56E−34 | Chr18 | 34726138 |
| rs11663052 | rs4453582 | 1 | 1 | 9.56E−34 | Chr18 | 34726377 |
| rs4239413 | rs4453582 | 1 | 1 | 9.56E−34 | Chr18 | 34727796 |
| rs6507286 | rs4453582 | 1 | 0.362637 | 3.83E−14 | Chr18 | 34728231 |
| rs12454739 | rs4453582 | 1 | 0.362637 | 3.83E−14 | Chr18 | 34729996 |
| rs12457494 | rs4453582 | 1 | 0.210054 | 3.31E−09 | Chr18 | 34730386 |
| rs11082114 | rs4453582 | 1 | 1 | 9.56E−34 | Chr18 | 34731949 |
| rs4133291 | rs4453582 | 1 | 0.362637 | 3.83E−14 | Chr18 | 34732242 |
| rs17596318 | rs4453582 | 1 | 1 | 2.29E−33 | Chr18 | 34734302 |
| rs4513170 | rs4453582 | 1 | 1 | 9.56E−34 | Chr18 | 34734590 |
| rs4453582 | rs4453582 | 1 | 1 | 0 | Chr18 | 34735189 |
| rs12958153 | rs4453582 | 1 | 1 | 9.56E−34 | Chr18 | 34735654 |
| rs4438376 | rs4453582 | 1 | 0.206349 | 2.80E−09 | Chr18 | 34736491 |
| rs4800060 | rs4453582 | 1 | 1 | 9.56E−34 | Chr18 | 34736841 |
| rs16971087 | rs4453582 | 1 | 0.960784 | 2.28E−31 | Chr18 | 34738607 |
| rs4374240 | rs4453582 | 1 | 0.235474 | 6.48E−08 | Chr18 | 34739954 |
| rs16971109 | rs4453582 | 1 | 0.960784 | 2.28E−31 | Chr18 | 34741246 |
| rs4396598 | rs4453582 | 1 | 0.960784 | 2.28E−31 | Chr18 | 34741542 |
| rs4433874 | rs4453582 | 1 | 0.960784 | 2.28E−31 | Chr18 | 34741559 |
| rs12608143 | rs4453582 | 0.946354 | 0.438655 | 4.16E−13 | Chr18 | 34746186 |
| rs17656091 | rs4453582 | 0.959795 | 0.920589 | 3.33E−26 | Chr18 | 34746308 |
| rs12961726 | rs4453582 | 1 | 0.960784 | 2.28E−31 | Chr18 | 34748540 |
| rs12956439 | rs4453582 | 1 | 0.960784 | 2.28E−31 | Chr18 | 34756148 |
| rs4583326 | rs4453582 | 1 | 0.362637 | 3.83E−14 | Chr18 | 34756878 |
| rs11664046 | rs4453582 | 1 | 0.960784 | 2.28E−31 | Chr18 | 34757357 |
| rs9945284 | rs4453582 | 0.785045 | 0.208936 | 3.08E−06 | Chr18 | 34759316 |
| rs17597991 | rs4453582 | 0.785045 | 0.208936 | 3.08E−06 | Chr18 | 34759853 |
| rs9951452 | rs4453582 | 0.785045 | 0.208936 | 3.08E−06 | Chr18 | 34760953 |
| rs9955069 | rs4453582 | 0.785045 | 0.208936 | 3.08E−06 | Chr18 | 34762541 |
| rs7236765 | rs4453582 | 0.785045 | 0.208936 | 3.08E−06 | Chr18 | 34764647 |
| rs5025765 | rs4453582 | 0.785045 | 0.208936 | 3.08E−06 | Chr18 | 34764938 |
| rs7228011 | rs4453582 | 0.906294 | 0.203798 | 7.25E−06 | Chr18 | 34951276 |
| rs2036709 | rs4453582 | 0.906294 | 0.203798 | 7.25E−06 | Chr18 | 34964595 |
| rs7237498 | rs4453582 | 0.906294 | 0.203798 | 7.25E−06 | Chr18 | 34965180 |
| rs4799499 | rs4453582 | 0.906294 | 0.203798 | 7.25E−06 | Chr18 | 34966397 |
| rs7234239 | rs4453582 | 0.906294 | 0.203798 | 7.25E−06 | Chr18 | 34968003 |
| rs9965186 | rs4453582 | 0.892016 | 0.221079 | 0.000036 | Chr18 | 34969836 |
| rs1396656 | rs4453582 | 0.906294 | 0.203798 | 7.25E−06 | Chr18 | 34971209 |
| rs4800084 | rs4453582 | 0.899483 | 0.201279 | 0.000018 | Chr18 | 34974065 |
| rs2861864 | rs4453582 | 0.906294 | 0.203798 | 7.25E−06 | Chr18 | 34975838 |
| rs1396655 | rs4453582 | 0.909382 | 0.212651 | 3.92E−06 | Chr18 | 34977323 |
| rs9959999 | rs4453582 | 0.906294 | 0.203798 | 7.25E−06 | Chr18 | 34980162 |
| rs4799501 | rs4453582 | 0.906294 | 0.203798 | 7.25E−06 | Chr18 | 34982156 |
| rs7245315 | rs4453582 | 0.906294 | 0.203798 | 7.25E−06 | Chr18 | 34982638 |
| rs7244771 | rs4453582 | 0.906294 | 0.203798 | 7.25E−06 | Chr18 | 34982812 |
| rs7229177 | rs4453582 | 0.876594 | 0.207409 | 0.000135 | Chr18 | 34988750 |
| rs12457561 | rs4453582 | 0.901259 | 0.205829 | 0.000014 | Chr18 | 34989073 |
| rs1509216 | rs4453582 | 0.906294 | 0.203798 | 7.25E−06 | Chr18 | 34991442 |
| rs1039806 | rs4453582 | 0.906294 | 0.203798 | 7.25E−06 | Chr18 | 34993034 |
| rs925238 | rs4453582 | 0.906294 | 0.203798 | 7.25E−06 | Chr18 | 34993367 |
| rs7238471 | rs4453582 | 0.906294 | 0.203798 | 7.25E−06 | Chr18 | 34995178 |
| rs4511606 | rs4453582 | 0.906294 | 0.203798 | 7.25E−06 | Chr18 | 35004955 |
| rs17401449 | rs2378199 | 1 | 0.298246 | 6.60E−07 | Chr20 | 31531606 |
| rs6088316 | rs2281695 | 0.557491 | 0.285898 | 1.49E−06 | Chr20 | 31890503 |
| rs2050209 | rs4911379 | 0.872652 | 0.450696 | 7.30E−12 | Chr20 | 31949197 |
| rs2050210 | rs4911379 | 0.473253 | 0.214714 | 3.75E−06 | Chr20 | 31949311 |
| rs4911371 | rs2284378 | 0.914085 | 0.391112 | 2.63E−08 | Chr20 | 31955563 |
| rs7409628 | rs4911379 | 0.872652 | 0.450696 | 7.30E−12 | Chr20 | 31971951 |
| rs2378026 | rs4911379 | 0.872652 | 0.450696 | 7.30E−12 | Chr20 | 31973312 |
| rs6120436 | rs4911379 | 0.945496 | 0.443424 | 7.49E−14 | Chr20 | 31977360 |
| rs6142046 | rs4911379 | 0.951506 | 0.572357 | 1.87E−17 | Chr20 | 31977722 |
| rs6059554 | rs4911379 | 0.957717 | 0.880011 | 6.25E−26 | Chr20 | 31977753 |
| rs6142047 | rs4911379 | 0.945496 | 0.443424 | 7.49E−14 | Chr20 | 31978047 |
| rs4911377 | rs4911379 | 0.945496 | 0.443424 | 7.49E−14 | Chr20 | 31979978 |
| rs6120440 | rs4911379 | 0.957717 | 0.880011 | 6.25E−26 | Chr20 | 31981246 |
| rs725478 | rs4911379 | 0.957717 | 0.880011 | 6.25E−26 | Chr20 | 31982015 |
| rs2143533 | rs4911379 | 0.945496 | 0.443424 | 7.49E−14 | Chr20 | 31982333 |
| rs6059563 | rs4911379 | 0.941976 | 0.44729 | 4.55E−13 | Chr20 | 31983008 |
| rs761238 | rs4911379 | 0.957717 | 0.880011 | 6.25E−26 | Chr20 | 31983649 |
| rs761236 | rs4911379 | 0.956366 | 0.877819 | 5.94E−25 | Chr20 | 31983712 |
| rs6059574 | rs4911379 | 0.957717 | 0.880011 | 6.25E−26 | Chr20 | 31986833 |
| rs2377955 | rs4911379 | 0.956463 | 0.909361 | 1.62E−23 | Chr20 | 31989031 |
| rs8119076 | rs4911379 | 0.951506 | 0.572357 | 1.87E−17 | Chr20 | 31989376 |
| rs6142050 | rs4911379 | 0.951506 | 0.572357 | 1.87E−17 | Chr20 | 31990789 |
| rs6059578 | rs4911379 | 0.95427 | 0.875378 | 2.47E−24 | Chr20 | 31991367 |
| rs6059581 | rs4911379 | 0.956399 | 0.87788 | 2.95E−25 | Chr20 | 31992049 |
| rs6142051 | rs4911379 | 1 | 0.655172 | 1.02E−21 | Chr20 | 31996679 |
| rs6579129 | rs2225837 | 0.807124 | 0.241351 | 1.18E−07 | Chr20 | 31997185 |
| rs761235 | rs2284378 | 1 | 1 | 1.04E−31 | Chr20 | 31998069 |
| rs4911379 | rs4911379 | 1 | 1 | 0 | Chr20 | 31998966 |
| rs973409 | rs2284378 | 1 | 1 | 4.02E−32 | Chr20 | 32000025 |
| rs6059586 | rs2284378 | 1 | 1 | 4.02E−32 | Chr20 | 32004162 |
| rs9753679 | rs2284378 | 1 | 0.508197 | 2.49E−17 | Chr20 | 32004521 |
| rs6142056 | rs2284378 | 1 | 0.651163 | 2.07E−21 | Chr20 | 32004816 |
| rs1319363 | rs2284378 | 1 | 1 | 4.02E−32 | Chr20 | 32005537 |
| rs6142058 | rs2284378 | 1 | 0.651163 | 2.07E−21 | Chr20 | 32006462 |
| rs13041173 | rs2225837 | 0.762508 | 0.239987 | 1.34E−07 | Chr20 | 32006475 |
| rs6059587 | rs2284378 | 1 | 1 | 4.02E−32 | Chr20 | 32006587 |
| rs4911138 | rs2284378 | 1 | 0.640414 | 1.08E−20 | Chr20 | 32011360 |
| rs4243974 | rs2284378 | 1 | 0.645161 | 1.09E−20 | Chr20 | 32011529 |
| rs6142061 | rs2284378 | 1 | 0.66805 | 5.62E−21 | Chr20 | 32011996 |
| rs6141432 | rs2284378 | 1 | 0.651163 | 2.07E−21 | Chr20 | 32012258 |
| rs4911381 | rs2284378 | 1 | 0.651163 | 2.07E−21 | Chr20 | 32014006 |
| rs1883707 | rs2284378 | 1 | 0.651163 | 2.07E−21 | Chr20 | 32016248 |
| rs4012234 | rs2284378 | 1 | 0.651163 | 2.07E−21 | Chr20 | 32016708 |
| rs4911382 | rs2284378 | 1 | 0.648188 | 2.83E−21 | Chr20 | 32016756 |
| rs6141436 | rs2284378 | 1 | 0.651163 | 2.07E−21 | Chr20 | 32018232 |
| rs4911383 | rs2284378 | 1 | 1 | 4.02E−32 | Chr20 | 32019440 |
| rs4911384 | rs2284378 | 1 | 0.651163 | 2.07E−21 | Chr20 | 32019468 |
| rs6142067 | rs2284378 | 1 | 0.642082 | 5.33E−21 | Chr20 | 32020233 |
| rs6059594 | rs619865 | 0.770904 | 0.212673 | 0.000339 | Chr20 | 32020438 |
| rs6059596 | rs2284378 | 1 | 1 | 4.02E−32 | Chr20 | 32020780 |
| rs6142068 | rs2284378 | 1 | 0.651163 | 2.07E−21 | Chr20 | 32020833 |
| rs6088360 | rs2284378 | 1 | 1 | 4.02E−32 | Chr20 | 32020850 |
| rs6088361 | rs2284378 | 1 | 1 | 4.02E−32 | Chr20 | 32020987 |
| rs10485503 | rs2284378 | 1 | 0.651163 | 2.07E−21 | Chr20 | 32021503 |
| rs4911385 | rs2284378 | 1 | 1 | 4.02E−32 | Chr20 | 32022254 |
| rs6142069 | rs2284378 | 1 | 0.645161 | 3.88E−21 | Chr20 | 32024203 |
| rs2064348 | rs2284378 | 1 | 0.363208 | 6.73E−11 | Chr20 | 32027739 |
| rs4911389 | rs2284378 | 1 | 1 | 4.02E−32 | Chr20 | 32028702 |
| rs4911393 | rs2284378 | 1 | 1 | 4.02E−32 | Chr20 | 32034587 |
| rs11697165 | rs2284378 | 1 | 0.421053 | 2.81E−15 | Chr20 | 32036945 |
| rs6057961 | rs2284378 | 1 | 1 | 4.02E−32 | Chr20 | 32037472 |
| rs6142078 | rs2284378 | 1 | 0.407895 | 4.55E−15 | Chr20 | 32037561 |
| rs1007090 | rs2284378 | 1 | 1 | 4.02E−32 | Chr20 | 32046532 |
| rs2284378 | rs2284378 | 1 | 1 | 0 | Chr20 | 32051756 |
| rs2300204 | rs2284378 | 1 | 1 | 2.08E−31 | Chr20 | 32052434 |
| rs2268079 | rs2284378 | 1 | 0.651163 | 2.07E−21 | Chr20 | 32060411 |
| rs2268080 | rs2284378 | 1 | 1 | 5.50E−32 | Chr20 | 32061377 |
| rs2268082 | rs2284378 | 1 | 0.440678 | 5.64E−16 | Chr20 | 32067588 |
| rs13043392 | rs2225837 | 0.78093 | 0.280202 | 9.32E−09 | Chr20 | 32070396 |
| rs1555075 | rs2284378 | 1 | 1 | 4.02E−32 | Chr20 | 32074062 |
| rs4911145 | rs4911414 | 1 | 0.673469 | 1.31E−21 | Chr20 | 32078884 |
| rs6120487 | rs2284378 | 1 | 0.958217 | 6.77E−29 | Chr20 | 32086269 |
| rs4911399 | rs2284378 | 1 | 0.63725 | 1.49E−20 | Chr20 | 32087079 |
| rs1883524 | rs2284378 | 1 | 0.957746 | 6.86E−30 | Chr20 | 32089768 |
| rs2268084 | rs2284378 | 1 | 0.429752 | 1.01E−15 | Chr20 | 32095049 |
| rs4911146 | rs2284378 | 1 | 1 | 4.02E−32 | Chr20 | 32103708 |
| rs3787230 | rs2284378 | 1 | 1 | 4.02E−32 | Chr20 | 32103933 |
| rs6059649 | rs2284378 | 1 | 1 | 4.02E−32 | Chr20 | 32107538 |
| rs2284386 | rs2284378 | 1 | 1 | 4.02E−32 | Chr20 | 32108057 |
| rs2284387 | rs2284378 | 1 | 1 | 4.02E−32 | Chr20 | 32108249 |
| rs6059651 | rs2284378 | 1 | 1 | 1.42E−31 | Chr20 | 32108713 |
| rs8119937 | rs2284378 | 1 | 1 | 4.02E−32 | Chr20 | 32109212 |
| rs909884 | rs2284378 | 1 | 1 | 2.85E−31 | Chr20 | 32109724 |
| rs2268086 | rs2284378 | 1 | 0.651163 | 2.07E−21 | Chr20 | 32112399 |
| rs2284388 | rs2284378 | 1 | 1 | 2.18E−30 | Chr20 | 32119402 |
| rs2284389 | rs2284378 | 1 | 1 | 4.02E−32 | Chr20 | 32120502 |
| rs932388 | rs2284378 | 1 | 0.648188 | 2.83E−21 | Chr20 | 32121039 |
| rs6057974 | rs4911414 | 0.952304 | 0.661013 | 6.08E−19 | Chr20 | 32124426 |
| rs2268089 | rs2284378 | 1 | 1 | 4.02E−32 | Chr20 | 32130959 |
| rs4911405 | rs2284378 | 1 | 1 | 4.02E−32 | Chr20 | 32138628 |
| rs6120513 | rs2284378 | 0.851576 | 0.469459 | 2.52E−13 | Chr20 | 32139297 |
| rs6059662 | rs2284378 | 1 | 0.957865 | 1.01E−29 | Chr20 | 32139388 |
| rs11700255 | rs2225837 | 0.826696 | 0.310471 | 4.09E−09 | Chr20 | 32145986 |
| rs4911407 | rs2284378 | 1 | 1 | 2.08E−31 | Chr20 | 32147226 |
| rs2235596 | rs2284378 | 1 | 1 | 4.02E−32 | Chr20 | 32147970 |
| rs6142096 | rs2284378 | 1 | 0.429752 | 1.01E−15 | Chr20 | 32150319 |
| rs11696338 | rs2284378 | 1 | 0.651163 | 1.65E−20 | Chr20 | 32151272 |
| rs6142100 | rs2284378 | 1 | 0.425957 | 3.04E−15 | Chr20 | 32161200 |
| rs6087557 | rs2284378 | 1 | 1 | 4.02E−32 | Chr20 | 32161936 |
| rs4911408 | rs2284378 | 1 | 1 | 4.02E−32 | Chr20 | 32164227 |
| rs6142102 | rs4911414 | 1 | 1 | 1.48E−32 | Chr20 | 32168288 |
| rs2223553 | rs4911414 | 1 | 0.655172 | 1.02E−21 | Chr20 | 32173105 |
| rs2206448 | rs4911414 | 1 | 1 | 1.48E−32 | Chr20 | 32173186 |
| rs4911410 | rs4911414 | 1 | 1 | 1.48E−32 | Chr20 | 32174635 |
| rs2378078 | rs4911414 | 1 | 0.830329 | 3.09E−24 | Chr20 | 32178389 |
| rs12480839 | rs4911414 | 1 | 1 | 1.48E−32 | Chr20 | 32191091 |
| rs4911414 | rs4911414 | 1 | 1 | 0 | Chr20 | 32193105 |
| rs1015362 | rs4911414 | 0.851077 | 0.581184 | 1.51E−15 | Chr20 | 32202273 |
| rs1015361 | rs4911414 | 1 | 0.69378 | 1.58E−20 | Chr20 | 32202347 |
| rs6142129 | rs2284378 | 0.518391 | 0.257625 | 3.32E−07 | Chr20 | 32283532 |
| rs6088466 | rs2225837 | 1 | 0.45098 | 4.85E−16 | Chr20 | 32377195 |
| rs1205344 | rs2225837 | 1 | 1 | 1.81E−37 | Chr20 | 32381959 |
| rs1205342 | rs2225837 | 1 | 1 | 1.81E−37 | Chr20 | 32385503 |
| rs1205340 | rs2225837 | 1 | 1 | 1.81E−37 | Chr20 | 32387532 |
| rs1205339 | rs2281695 | 1 | 1 | 5.10E−26 | Chr20 | 32388628 |
| rs1205338 | rs2225837 | 1 | 1 | 1.81E−37 | Chr20 | 32389286 |
| rs1205337 | rs2225837 | 1 | 1 | 3.16E−37 | Chr20 | 32389702 |
| rs1205336 | rs2225837 | 1 | 1 | 1.81E−37 | Chr20 | 32389997 |
| rs2378134 | rs2225837 | 1 | 0.966555 | 6.06E−35 | Chr20 | 32401746 |
| rs12624640 | rs2225837 | 1 | 0.790105 | 3.97E−28 | Chr20 | 32415786 |
| rs6087577 | rs2225837 | 1 | 1 | 1.81E−37 | Chr20 | 32419084 |
| rs3746455 | rs2225837 | 1 | 1 | 1.81E−37 | Chr20 | 32420877 |
| rs4911153 | rs2225837 | 1 | 1 | 1.81E−37 | Chr20 | 32425208 |
| rs2184836 | rs2225837 | 1 | 1 | 3.16E−37 | Chr20 | 32427030 |
| rs6141465 | rs2225837 | 1 | 1 | 1.81E−37 | Chr20 | 32430975 |
| rs1890000 | rs2225837 | 1 | 1 | 1.81E−37 | Chr20 | 32431973 |
| rs11167234 | rs2225837 | 1 | 0.454669 | 8.39E−16 | Chr20 | 32447280 |
| rs6142157 | rs2225837 | 1 | 1 | 1.81E−37 | Chr20 | 32447833 |
| rs6088483 | rs2225837 | 1 | 1 | 7.46E−37 | Chr20 | 32448375 |
| rs4142007 | rs2225837 | 1 | 1 | 9.38E−36 | Chr20 | 32453711 |
| rs6087580 | rs2225837 | 1 | 1 | 7.46E−37 | Chr20 | 32456253 |
| rs6088488 | rs2225837 | 1 | 1 | 1.81E−37 | Chr20 | 32456292 |
| rs6142159 | rs2225837 | 1 | 0.678284 | 1.84E−21 | Chr20 | 32456696 |
| rs6579165 | rs2225837 | 1 | 1 | 7.46E−37 | Chr20 | 32458376 |
| rs4911154 | rs2281695 | 1 | 1 | 5.10E−26 | Chr20 | 32459762 |
| rs3761147 | rs2225837 | 1 | 1 | 1.81E−37 | Chr20 | 32460350 |
| rs4911420 | rs2225837 | 1 | 1 | 1.81E−37 | Chr20 | 32462315 |
| rs4911421 | rs2225837 | 1 | 1 | 7.29E−36 | Chr20 | 32462609 |
| rs4911423 | rs2225837 | 1 | 1 | 1.81E−37 | Chr20 | 32463047 |
| rs2225837 | rs2225837 | 1 | 1 | 0 | Chr20 | 32469295 |
| rs4277599 | rs2225837 | 1 | 1 | 1.81E−37 | Chr20 | 32472566 |
| rs2424992 | rs2225837 | 1 | 1 | 1.81E−37 | Chr20 | 32475721 |
| rs6059827 | rs2225837 | 1 | 1 | 1.81E−37 | Chr20 | 32480019 |
| rs6088498 | rs2225837 | 1 | 1 | 1.81E−37 | Chr20 | 32484107 |
| rs6120644 | rs2225837 | 1 | 1 | 7.46E−37 | Chr20 | 32487471 |
| rs6579167 | rs2225837 | 1 | 0.25745 | 8.36E−11 | Chr20 | 32496576 |
| rs3736762 | rs2225837 | 1 | 1 | 1.81E−37 | Chr20 | 32500997 |
| rs6088502 | rs2225837 | 0.965458 | 0.928552 | 9.02E−29 | Chr20 | 32501305 |
| rs6120650 | rs2225837 | 1 | 1 | 1.81E−37 | Chr20 | 32503634 |
| rs6058051 | rs2225837 | 1 | 1 | 1.81E−37 | Chr20 | 32508523 |
| rs6058052 | rs2225837 | 1 | 0.961117 | 2.28E−31 | Chr20 | 32510745 |
| rs6059851 | rs2225837 | 1 | 1 | 4.80E−35 | Chr20 | 32511606 |
| rs7269526 | rs2281695 | 1 | 1 | 5.10E−26 | Chr20 | 32516954 |
| rs6059856 | rs2225837 | 1 | 1 | 1.81E−37 | Chr20 | 32521615 |
| rs6059860 | rs2225837 | 1 | 1 | 1.81E−37 | Chr20 | 32527491 |
| rs6087588 | rs2225837 | 1 | 1 | 1.81E−37 | Chr20 | 32530030 |
| rs6059866 | rs2225837 | 1 | 1 | 1.81E−37 | Chr20 | 32539471 |
| rs6059867 | rs2225837 | 1 | 1 | 1.81E−37 | Chr20 | 32541764 |
| rs6059868 | rs2225837 | 1 | 1 | 1.81E−37 | Chr20 | 32543121 |
| rs6120663 | rs2225837 | 1 | 0.816934 | 4.41E−29 | Chr20 | 32545567 |
| rs6059875 | rs2225837 | 1 | 1 | 1.81E−37 | Chr20 | 32549751 |
| rs6059878 | rs2225837 | 1 | 1 | 1.81E−37 | Chr20 | 32550827 |
| rs6059880 | rs2225837 | 1 | 1 | 7.46E−37 | Chr20 | 32552330 |
| rs6088512 | rs2225837 | 1 | 1 | 1.81E−37 | Chr20 | 32559552 |
| rs6059887 | rs2225837 | 1 | 1 | 1.81E−37 | Chr20 | 32564763 |
| rs6058070 | rs2225837 | 1 | 1 | 7.46E−37 | Chr20 | 32567182 |
| rs2424993 | rs2225837 | 1 | 0.463087 | 3.50E−16 | Chr20 | 32568253 |
| rs6120669 | rs2225837 | 1 | 1 | 1.81E−37 | Chr20 | 32568689 |
| rs6059892 | rs2225837 | 1 | 1 | 1.81E−37 | Chr20 | 32569427 |
| rs6059893 | rs2225837 | 1 | 1 | 1.49E−33 | Chr20 | 32569558 |
| rs6088515 | rs2281695 | 1 | 1 | 5.10E−26 | Chr20 | 32573703 |
| rs2378205 | rs2225837 | 1 | 1 | 1.81E−37 | Chr20 | 32574464 |
| rs1122174 | rs2281695 | 1 | 1 | 4.37E−25 | Chr20 | 32574507 |
| rs6059896 | rs2225837 | 1 | 1 | 1.81E−37 | Chr20 | 32575444 |
| rs6059897 | rs2225837 | 1 | 1 | 7.46E−37 | Chr20 | 32576885 |
| rs6087592 | rs2225837 | 1 | 1 | 1.81E−37 | Chr20 | 32578164 |
| rs6058073 | rs2225837 | 1 | 1 | 1.81E−37 | Chr20 | 32580144 |
| rs2281695 | rs2281695 | 1 | 1 | 0 | Chr20 | 32592825 |
| rs6059908 | rs2281695 | 1 | 0.944904 | 3.09E−24 | Chr20 | 32595820 |
| rs6088519 | rs2225837 | 0.854221 | 0.273064 | 3.85E−08 | Chr20 | 32595852 |
| rs6088520 | rs2225837 | 0.92732 | 0.751157 | 1.15E−24 | Chr20 | 32596025 |
| rs2424994 | rs6060034 | 0.866122 | 0.596016 | 9.77E−14 | Chr20 | 32596578 |
| rs6059909 | rs6059909 | 1 | 1 | 0 | Chr20 | 32603352 |
| rs4911430 | rs2378199 | 1 | 1 | 7.33E−24 | Chr20 | 32609065 |
| rs2144956 | rs2378199 | 1 | 1 | 5.87E−23 | Chr20 | 32609529 |
| rs6059916 | rs2378199 | 1 | 1 | 5.87E−23 | Chr20 | 32612522 |
| rs6059918 | rs6059909 | 1 | 1 | 9.94E−37 | Chr20 | 32614164 |
| rs6141482 | rs6059909 | 1 | 0.526835 | 3.19E−18 | Chr20 | 32620184 |
| rs764597 | rs6059909 | 1 | 1 | 7.04E−38 | Chr20 | 32624886 |
| rs6142199 | rs2378199 | 1 | 1 | 2.91E−23 | Chr20 | 32625959 |
| rs2889849 | rs2378199 | 1 | 1 | 8.64E−24 | Chr20 | 32627938 |
| rs6059926 | rs6059909 | 1 | 1 | 2.47E−34 | Chr20 | 32628465 |
| rs6059928 | rs2378199 | 1 | 1 | 7.33E−24 | Chr20 | 32631010 |
| rs6088529 | rs6059909 | 1 | 0.526835 | 7.17E−19 | Chr20 | 32634413 |
| rs1884669 | rs6059909 | 1 | 1 | 1.30E−37 | Chr20 | 32634948 |
| rs932542 | rs2378199 | 1 | 1 | 5.87E−23 | Chr20 | 32635029 |
| rs4302281 | rs2378199 | 1 | 1 | 7.33E−24 | Chr20 | 32635306 |
| rs910873 | rs2378199 | 1 | 0.571865 | 2.01E−12 | Chr20 | 32635433 |
| rs2295443 | rs2378199 | 1 | 1 | 5.87E−23 | Chr20 | 32637488 |
| rs2295444 | rs6059909 | 1 | 1 | 7.04E−38 | Chr20 | 32637544 |
| rs6059931 | rs2378199 | 0.86928 | 0.755648 | 1.79E−16 | Chr20 | 32638999 |
| rs6059932 | rs6059909 | 1 | 1 | 7.04E−38 | Chr20 | 32639127 |
| rs4564863 | rs2378199 | 1 | 1 | 7.33E−24 | Chr20 | 32643028 |
| rs17305573 | rs2378199 | 1 | 0.571865 | 2.22E−12 | Chr20 | 32643813 |
| rs6087605 | rs6059909 | 1 | 1 | 2.15E−36 | Chr20 | 32644285 |
| rs6059937 | rs2378199 | 1 | 1 | 7.33E−24 | Chr20 | 32649861 |
| rs2378199 | rs2378199 | 1 | 1 | 0 | Chr20 | 32650141 |
| rs6088536 | rs6059909 | 1 | 0.966942 | 1.29E−35 | Chr20 | 32652767 |
| rs7264012 | rs6059909 | 1 | 0.526835 | 7.17E−19 | Chr20 | 32654688 |
| rs6142201 | rs6059909 | 1 | 0.526835 | 7.17E−19 | Chr20 | 32655413 |
| rs6141488 | rs6059909 | 1 | 0.526835 | 7.17E−19 | Chr20 | 32656407 |
| rs721613 | rs6059909 | 1 | 0.526835 | 1.51E−18 | Chr20 | 32657089 |
| rs6058089 | rs2378199 | 1 | 1 | 8.64E−24 | Chr20 | 32657918 |
| rs6087606 | rs6059909 | 1 | 0.526835 | 7.17E−19 | Chr20 | 32658087 |
| rs6088537 | rs6059909 | 1 | 0.526835 | 7.17E−19 | Chr20 | 32659269 |
| rs6087607 | rs2378199 | 1 | 1 | 7.33E−24 | Chr20 | 32661150 |
| rs6058091 | rs2378199 | 1 | 0.922481 | 8.12E−19 | Chr20 | 32662051 |
| rs6579178 | rs6059909 | 1 | 0.52 | 3.31E−18 | Chr20 | 32667213 |
| rs6088543 | rs6059909 | 1 | 0.541057 | 4.82E−19 | Chr20 | 32669852 |
| rs6142206 | rs6059909 | 1 | 0.791563 | 2.55E−28 | Chr20 | 32675716 |
| rs6087609 | rs6059909 | 1 | 0.526835 | 7.17E−19 | Chr20 | 32678202 |
| rs11904852 | rs6059909 | 1 | 0.526835 | 7.17E−19 | Chr20 | 32680425 |
| rs2378249 | rs2378199 | 1 | 1 | 7.33E−24 | Chr20 | 32681751 |
| rs6059956 | rs6059909 | 1 | 1 | 7.04E−38 | Chr20 | 32683731 |
| rs6142210 | rs6059909 | 0.818291 | 0.410698 | 1.24E−12 | Chr20 | 32686673 |
| rs6088552 | rs6059909 | 0.818291 | 0.410698 | 1.24E−12 | Chr20 | 32690152 |
| rs7274854 | rs6059909 | 0.816749 | 0.40411 | 2.48E−12 | Chr20 | 32692333 |
| rs7269596 | rs6059909 | 0.818291 | 0.410698 | 1.24E−12 | Chr20 | 32692724 |
| rs6087612 | rs6059909 | 0.818291 | 0.410698 | 1.24E−12 | Chr20 | 32694483 |
| rs2068474 | rs2378199 | 1 | 1 | 7.33E−24 | Chr20 | 32694740 |
| rs6059961 | rs2378199 | 1 | 1 | 7.33E−24 | Chr20 | 32695151 |
| rs4911158 | rs6059909 | 0.818291 | 0.410698 | 1.24E−12 | Chr20 | 32703173 |
| rs6059969 | rs2378199 | 1 | 1 | 7.33E−24 | Chr20 | 32708945 |
| rs6088565 | rs6059909 | 0.818291 | 0.410698 | 1.24E−12 | Chr20 | 32718647 |
| rs6088567 | rs6059909 | 0.808644 | 0.372087 | 1.87E−11 | Chr20 | 32727533 |
| rs6088568 | rs6059909 | 0.818291 | 0.410698 | 1.24E−12 | Chr20 | 32729632 |
| rs6088569 | rs6059909 | 0.755614 | 0.36936 | 1.39E−09 | Chr20 | 32731860 |
| rs6088575 | rs6059909 | 0.818291 | 0.410698 | 1.24E−12 | Chr20 | 32737064 |
| rs2378251 | rs6059909 | 0.818291 | 0.410698 | 1.24E−12 | Chr20 | 32740411 |
| rs2889855 | rs6059909 | 0.818291 | 0.410698 | 1.24E−12 | Chr20 | 32740514 |
| rs6088578 | rs6059909 | 0.807004 | 0.370579 | 1.80E−11 | Chr20 | 32745265 |
| rs6088580 | rs6059909 | 0.961537 | 0.853689 | 1.01E−24 | Chr20 | 32748714 |
| rs6087619 | rs6059909 | 0.958476 | 0.610703 | 4.19E−20 | Chr20 | 32752172 |
| rs910869 | rs6059909 | 0.957877 | 0.614532 | 7.32E−20 | Chr20 | 32756438 |
| rs910870 | rs6059909 | 0.958639 | 0.615121 | 2.29E−20 | Chr20 | 32756554 |
| rs6060001 | rs6059909 | 0.963019 | 0.758999 | 3.05E−25 | Chr20 | 32758014 |
| rs6060003 | rs6059909 | 0.963019 | 0.758999 | 3.05E−25 | Chr20 | 32758606 |
| rs6060009 | rs6060034 | 1 | 1 | 1.31E−24 | Chr20 | 32767635 |
| rs6119512 | rs6059909 | 0.958639 | 0.615121 | 2.29E−20 | Chr20 | 32770565 |
| rs6060017 | rs6060034 | 1 | 1 | 1.31E−24 | Chr20 | 32776703 |
| rs6088590 | rs6059909 | 0.958639 | 0.615121 | 2.29E−20 | Chr20 | 32777227 |
| rs4911441 | rs6059909 | 0.514371 | 0.200864 | 0.000017 | Chr20 | 32780207 |
| rs6087623 | rs6059909 | 0.957213 | 0.611544 | 1.46E−19 | Chr20 | 32781323 |
| rs2295352 | rs6059909 | 0.686008 | 0.299477 | 3.31E−09 | Chr20 | 32783716 |
| rs6060025 | rs6060034 | 1 | 0.883721 | 1.75E−21 | Chr20 | 32790537 |
| rs6119516 | rs6059909 | 0.958639 | 0.615121 | 2.29E−20 | Chr20 | 32791685 |
| rs3787223 | rs6060034 | 1 | 1 | 1.31E−24 | Chr20 | 32795046 |
| rs3787222 | rs6059909 | 0.957877 | 0.614532 | 7.32E−20 | Chr20 | 32796701 |
| rs910871 | rs6060034 | 1 | 1 | 1.31E−24 | Chr20 | 32796869 |
| rs6120708 | rs6059909 | 0.958639 | 0.615121 | 2.29E−20 | Chr20 | 32799187 |
| rs3787220 | rs6060034 | 1 | 1 | 1.31E−24 | Chr20 | 32801412 |
| rs1884431 | rs2378199 | 1 | 1 | 5.84E−20 | Chr20 | 32802246 |
| rs6060030 | rs6060034 | 1 | 1 | 1.31E−24 | Chr20 | 32803974 |
| rs1884432 | rs6060034 | 1 | 1 | 3.82E−23 | Chr20 | 32806100 |
| rs6087625 | rs6059909 | 0.958639 | 0.615121 | 2.29E−20 | Chr20 | 32806138 |
| rs6088594 | rs6060034 | 1 | 1 | 1.31E−24 | Chr20 | 32806818 |
| rs1998028 | rs6059909 | 0.958639 | 0.615121 | 2.29E−20 | Chr20 | 32808256 |
| rs959829 | rs6059909 | 0.686008 | 0.299477 | 3.31E−09 | Chr20 | 32809708 |
| rs6060034 | rs6060034 | 1 | 1 | 0 | Chr20 | 32815525 |
| rs4911442 | rs2378199 | 1 | 0.630573 | 9.69E−11 | Chr20 | 32818707 |
| rs2295353 | rs6059909 | 0.957213 | 0.611544 | 1.46E−19 | Chr20 | 32820172 |
| rs6058115 | rs6060034 | 1 | 1 | 1.31E−24 | Chr20 | 32822058 |
| rs2180276 | rs6059909 | 0.957213 | 0.611544 | 1.46E−19 | Chr20 | 32824446 |
| rs6060043 | rs6060034 | 1 | 1 | 1.31E−24 | Chr20 | 32828245 |
| rs6060047 | rs6060034 | 1 | 1 | 1.31E−24 | Chr20 | 32831061 |
| rs6120730 | rs6059909 | 0.955811 | 0.601539 | 1.71E−18 | Chr20 | 32848763 |
| rs7271289 | rs6060034 | 1 | 1 | 1.31E−24 | Chr20 | 32860964 |
| rs1018447 | rs6059909 | 0.686008 | 0.299477 | 3.31E−09 | Chr20 | 32865368 |
| rs2425003 | rs6060034 | 1 | 0.934676 | 2.47E−21 | Chr20 | 32867245 |
| rs6120739 | rs6059909 | 0.686008 | 0.299477 | 3.31E−09 | Chr20 | 32868888 |
| rs2253484 | rs6059909 | 0.686008 | 0.299477 | 3.31E−09 | Chr20 | 32868924 |
| rs2889861 | rs6059909 | 0.686008 | 0.299477 | 3.31E−09 | Chr20 | 32869325 |
| rs6060064 | rs6059909 | 0.686008 | 0.299477 | 3.31E−09 | Chr20 | 32870000 |
| rs6087632 | rs6059909 | 0.957213 | 0.611544 | 1.46E−19 | Chr20 | 32871365 |
| rs2425005 | rs6059909 | 0.686008 | 0.299477 | 3.31E−09 | Chr20 | 32871376 |
| rs6088618 | rs6059909 | 0.963019 | 0.758999 | 3.05E−25 | Chr20 | 32873011 |
| rs12626122 | rs6059909 | 0.955557 | 0.584882 | 1.12E−18 | Chr20 | 32889461 |
| rs12625149 | rs6059909 | 0.95742 | 0.588736 | 4.20E−19 | Chr20 | 32889473 |
| rs17092148 | rs6060034 | 1 | 1 | 1.31E−24 | Chr20 | 32898822 |
| rs6088624 | rs6059909 | 0.954315 | 0.590875 | 2.13E−18 | Chr20 | 32900513 |
| rs2076668 | rs6059909 | 0.958639 | 0.615121 | 2.29E−20 | Chr20 | 32901282 |
| rs6119535 | rs6059909 | 0.958639 | 0.615121 | 2.29E−20 | Chr20 | 32905799 |
| rs6120747 | rs6059909 | 0.957213 | 0.611544 | 1.46E−19 | Chr20 | 32913430 |
| rs6119536 | rs6059909 | 0.686008 | 0.299477 | 3.31E−09 | Chr20 | 32913702 |
| rs11546155 | rs6060034 | 1 | 0.93808 | 3.83E−22 | Chr20 | 32914809 |
| rs17122844 | rs6060034 | 1 | 0.780822 | 2.07E−19 | Chr20 | 32916261 |
| rs7263157 | rs6059909 | 0.681577 | 0.304791 | 4.35E−09 | Chr20 | 32922788 |
| rs6120750 | rs6059909 | 0.957213 | 0.611544 | 1.46E−19 | Chr20 | 32928950 |
| rs6088635 | rs6059909 | 0.686008 | 0.299477 | 3.31E−09 | Chr20 | 32930162 |
| rs1013677 | rs6059909 | 0.686008 | 0.299477 | 3.31E−09 | Chr20 | 32932454 |
| rs4911163 | rs6059909 | 0.686008 | 0.299477 | 3.31E−09 | Chr20 | 32934355 |
| rs6088640 | rs6059909 | 0.686008 | 0.299477 | 3.31E−09 | Chr20 | 32936170 |
| rs6058137 | rs6059909 | 0.686008 | 0.299477 | 3.31E−09 | Chr20 | 32938735 |
| rs8116657 | rs6059909 | 0.686008 | 0.299477 | 3.31E−09 | Chr20 | 32940135 |
| rs1060615 | rs6059909 | 0.678783 | 0.290404 | 8.39E−09 | Chr20 | 32942042 |
| rs4911164 | rs6059909 | 0.686008 | 0.299477 | 3.31E−09 | Chr20 | 32943149 |
| rs6087644 | rs6059909 | 0.677804 | 0.281784 | 1.08E−08 | Chr20 | 32944578 |
| rs6088642 | rs6059909 | 0.686008 | 0.299411 | 3.31E−09 | Chr20 | 32946847 |
| rs6119542 | rs6059909 | 0.686008 | 0.299477 | 3.31E−09 | Chr20 | 32948206 |
| rs6120757 | rs6059909 | 0.686008 | 0.299477 | 3.31E−09 | Chr20 | 32952432 |
| rs6120758 | rs6059909 | 0.686008 | 0.299477 | 3.31E−09 | Chr20 | 32956184 |
| rs7266550 | rs6059909 | 0.686008 | 0.299477 | 3.31E−09 | Chr20 | 32959171 |
| rs6088646 | rs6059909 | 0.686008 | 0.299477 | 3.31E−09 | Chr20 | 32969598 |
| rs2223881 | rs6059909 | 0.686008 | 0.299477 | 3.31E−09 | Chr20 | 32970124 |
| rs2076667 | rs6059909 | 0.686964 | 0.308723 | 2.52E−09 | Chr20 | 32970625 |
| rs3746450 | rs6059909 | 0.686964 | 0.308723 | 2.52E−09 | Chr20 | 32972249 |
| rs3818273 | rs6059909 | 0.686964 | 0.308723 | 2.52E−09 | Chr20 | 32972936 |
| rs2273683 | rs6059909 | 0.681577 | 0.304791 | 4.35E−09 | Chr20 | 32973184 |
| rs4911449 | rs6059909 | 0.686008 | 0.299477 | 3.31E−09 | Chr20 | 32975897 |
| rs4911450 | rs6059909 | 0.686964 | 0.308723 | 2.52E−09 | Chr20 | 32976067 |
| rs4911451 | rs6059909 | 0.677762 | 0.296476 | 1.67E−08 | Chr20 | 32976127 |
| rs6088650 | rs6059909 | 0.686964 | 0.308723 | 2.52E−09 | Chr20 | 32978126 |
| rs725521 | rs6059909 | 0.681577 | 0.304791 | 4.35E−09 | Chr20 | 32979732 |
| rs6087653 | rs6059909 | 0.686008 | 0.299477 | 3.31E−09 | Chr20 | 32985715 |
| rs2236270 | rs6059909 | 0.958639 | 0.615121 | 2.29E−20 | Chr20 | 32986816 |
| rs2236271 | rs6059909 | 0.686008 | 0.299477 | 3.31E−09 | Chr20 | 32987501 |
| rs7265992 | rs6060034 | 1 | 1 | 7.02E−22 | Chr20 | 32989068 |
| rs6088655 | rs6059909 | 0.686008 | 0.299477 | 3.31E−09 | Chr20 | 32991499 |
| rs2273684 | rs6059909 | 0.963019 | 0.758999 | 3.05E−25 | Chr20 | 32993427 |
| rs734111 | rs6059909 | 0.679787 | 0.290056 | 1.16E−08 | Chr20 | 32997397 |
| rs6060124 | rs2378199 | 0.763485 | 0.279504 | 1.87E−07 | Chr20 | 33000558 |
| rs6060127 | rs2378199 | 0.754751 | 0.225505 | 7.83E−07 | Chr20 | 33002660 |
| rs2025096 | rs6059909 | 0.912629 | 0.206619 | 5.79E−07 | Chr20 | 33003661 |
| rs6088659 | rs2225837 | 0.907679 | 0.214419 | 3.42E−07 | Chr20 | 33006266 |
| rs6088660 | rs2378199 | 0.734668 | 0.213368 | 4.79E−06 | Chr20 | 33006557 |
| rs3761144 | rs6059909 | 0.667273 | 0.33783 | 8.16E−10 | Chr20 | 33007736 |
| rs6060130 | rs6059909 | 0.671175 | 0.330939 | 6.77E−10 | Chr20 | 33012980 |
| rs4911165 | rs2378199 | 0.751515 | 0.215069 | 1.28E−06 | Chr20 | 33014043 |
| rs6088664 | rs6059909 | 0.666819 | 0.328135 | 9.69E−10 | Chr20 | 33014761 |
| rs7263251 | rs2378199 | 0.811907 | 0.243623 | 4.47E−07 | Chr20 | 33017981 |
| rs6058149 | rs2378199 | 0.829629 | 0.248804 | 1.67E−07 | Chr20 | 33018149 |
| rs6058150 | rs2378199 | 0.751515 | 0.215069 | 1.28E−06 | Chr20 | 33020478 |
| rs6060133 | rs2378199 | 0.801895 | 0.246274 | 8.90E−07 | Chr20 | 33021672 |
| rs6120778 | rs6059909 | 0.628794 | 0.345374 | 4.23E−10 | Chr20 | 33028830 |
| rs6060140 | rs6059909 | 0.508092 | 0.25784 | 6.34E−07 | Chr20 | 33030168 |
| rs1885120 | rs2378199 | 1 | 0.459459 | 4.28E−10 | Chr20 | 33040650 |
| rs1885114 | rs6059909 | 0.628794 | 0.345374 | 4.23E−10 | Chr20 | 33041022 |
| rs2425012 | rs6059909 | 0.625348 | 0.353672 | 4.53E−10 | Chr20 | 33045616 |
| rs3746438 | rs6059909 | 0.615312 | 0.346415 | 1.01E−09 | Chr20 | 33047950 |
| rs6058154 | rs6059909 | 0.628794 | 0.345374 | 4.23E−10 | Chr20 | 33049495 |
| rs3736802 | rs6059909 | 0.532906 | 0.230182 | 9.96E−07 | Chr20 | 33067703 |
| rs6141526 | rs6059909 | 0.626414 | 0.342764 | 8.06E−10 | Chr20 | 33078916 |
| rs6142280 | rs6059909 | 0.681527 | 0.369087 | 5.71E−09 | Chr20 | 33085903 |
| rs13042358 | rs6059909 | 0.621548 | 0.33932 | 1.19E−09 | Chr20 | 33098140 |
| rs2038504 | rs6059909 | 0.6255 | 0.338411 | 1.77E−09 | Chr20 | 33111848 |
| rs6060199 | rs6059909 | 0.616784 | 0.314515 | 6.50E−09 | Chr20 | 33118434 |
| rs6142294 | rs6059909 | 0.639624 | 0.328735 | 3.55E−09 | Chr20 | 33135158 |
| rs3746427 | rs6059909 | 0.510478 | 0.251973 | 1.18E−07 | Chr20 | 33194125 |
| rs6088747 | rs6059909 | 0.510478 | 0.251973 | 1.18E−07 | Chr20 | 33218265 |
| rs2069948 | rs6059909 | 0.529002 | 0.272039 | 6.00E−08 | Chr20 | 33226150 |
| rs2069952 | rs6059909 | 0.529002 | 0.272039 | 6.00E−08 | Chr20 | 33227612 |
| rs9574 | rs6059909 | 0.529002 | 0.272039 | 6.00E−08 | Chr20 | 33228293 |
| rs1415774 | rs6059909 | 0.510478 | 0.251973 | 1.18E−07 | Chr20 | 33229277 |
| rs2065979 | rs6059909 | 0.510478 | 0.251973 | 1.18E−07 | Chr20 | 33231351 |
| rs6060285 | rs6059909 | 0.510478 | 0.251973 | 1.18E−07 | Chr20 | 33234148 |
| rs6058202 | rs6059909 | 0.505552 | 0.244411 | 4.24E−07 | Chr20 | 33241644 |
| rs633198 | rs6059909 | 0.510478 | 0.251973 | 1.18E−07 | Chr20 | 33242095 |
| rs663550 | rs6059909 | 0.501045 | 0.249375 | 1.90E−07 | Chr20 | 33242929 |
| rs4911478 | rs6059909 | 0.550514 | 0.277563 | 4.54E−08 | Chr20 | 33246286 |
| rs1577924 | rs6059909 | 0.510478 | 0.251973 | 1.18E−07 | Chr20 | 33247949 |
| rs6142324 | rs6059909 | 0.510478 | 0.251973 | 1.18E−07 | Chr20 | 33252704 |
| rs8114671 | rs6059909 | 0.510478 | 0.251973 | 1.18E−07 | Chr20 | 33252803 |
| rs2093058 | rs6059909 | 0.510478 | 0.251973 | 1.18E−07 | Chr20 | 33254942 |
| rs619865 | rs619865 | 1 | 1 | 0 | Chr20 | 33331111 |
| rs17421899 | rs619865 | 0.912916 | 0.766654 | 4.95E−14 | Chr20 | 33398852 |
| rs738703 | rs619865 | 0.884652 | 0.221861 | 1.65E−06 | Chr20 | 33654113 |
| rs2425067 | rs619865 | 0.912086 | 0.707504 | 2.95E−13 | Chr20 | 33671930 |

### Example 3

### Identification of Variants Associated with Melanoma

A follow-up analysis of variants associated with freckles and skin sensitivity to sun was performed. In particular, 484 individuals diagnosed with malignant melanoma cancer were assessed for the particular markers described in Example 1 and Example 2. The analysis revealed significant association of marker rs6060043 to melanoma, with an increased risk of heterozygous carriers of 39%, as indicated in Table 12. This marker is therefore useful for diagnosing a risk of, or a susceptibility to, melanoma. Malignant cutaneous melanoma was diagnosed according to ICD-10 classification, and obtained from the Icelandic Cancer Registry.

The marker shows correlation to sun sensitivity of the skin, to freckles and to red hair. This is consistent with the effect on melanoma susceptibility, since those sensitive to sun exposure are at increased risk of developing melanoma cancer. Furthermore, red hair is frequently associated with sun sensitive skin and freckles.

TABLE 12
Results for association of marker rs6060043 allele 2.

| Phenotype comparison | P-value | OR | f group 1 | N group 1 | f group 2 | N group 2 |
|---|---|---|---|---|---|---|
| Melanoma* vs controls | 6.1 × 10⁻⁵ | 1.39 | 0.211 | 484 | 0.161 | 27178 |
| red vs non-red hair | 1.9 × 10⁻⁵ | 1.43 | 0.209 | 502 | 0.156 | 6405 |
| sun sensitive vs not sun | 3.8 × 10⁻¹¹ | 1.38 | 0.19 | 2425 | 0.145 | 4221 |
| sensitive | | | | | | |
| freckles vs not freckles | 3.0 × 10⁻¹³ | 1.41 | 0.182 | 3648 | 0.137 | 3204 |
| freckles/sun sensitive vs | 2.7 × 10⁻¹⁸ | 1.69 | 0.206 | 1717 | 0.133 | 2357 |
| not freckles/not sun | | | | | | |
| sensitive | | | | | | |

*malignant cutaneous melanoma

Marker rs6060043 is located within a region of extensive linkage disequilibrium on chromosome 20q11.22 (FIG. 9). Several markers in the region are in strong LD with the marker, as indicated in Table 11, all of which could be used as surrogates for the marker. The region includes a number of genes, all of which are plausible candidates for the functional effect of this variant. One of these genes encodes for the Agouti Signaling Protein (ASIP). This gene is the human homologue of the mouse agouti gene which encodes a paracrine signaling molecule that causes hair follicle melanocytes to synthesize pheomelanin, a yellow pigment, instead of the black or brown pigment eumelanin. Consequently, agouti mice produce hairs with a subapical yellow band on an otherwise black or brown background when expressed during the midportion of hair growth. The coding region of the human gene is 85% identical to that of the mouse gene and has the potential to encode a protein of 132 amino acids with a consensus signal peptide.

The ASIP gene product interacts with the melanocyte receptor for alpha-melanocyte stimulating hormone (MC1R), and in transgenic mice expression of ASIP produced a yellow coat, and expression of ASP in cell culture blocked the MC1R-stimulated accumulation of cAMP in mouse melanoma cells. In mice and humans, binding of alpha-melanocyte-stimulating hormone to the melanocyte-stimulating-hormone receptor (MSHR), the protein product of the melanocortin-1 receptor (MC1R) gene, leads to the synthesis of eumelanin. The ASIP gene therefore is a possible candidate for the observed association of rs6060043 to melanoma and skin and hair pigmentation. The marker is located close to 500 kb distal to the ASIP gene on chromosome 20. It is possible that the marker is in linkage disequilibrium with another marker closer to, or within, the ASIP with functional consequences on gene expression of ASIP, or on the ASIP gene product itself. Alternatively, other the functional effect of rs6060043 is through other genes located in this region.

The present inventors have also found that marker rs1393350, which is shown herein as being associated to eye, hair and skin pigmentation, is also associated with melanoma (OR=1.21, p=0.0061), based on analysis of 483 cases and 27,140 population controls. This markers is therefore useful for determining a susceptibility to melanoma, as described herein.

### Example 4

### Further Investigation of Variants Associated with Human Pigmentation Patterns

The genome-wide scan for pigmentation variants was expanded to 5,130 individuals from Iceland. The findings of this discovery phase were followed up in 2,116 Icelanders and 1,214 Dutch individuals. We examined the association of sequence variants with pigmentation traits in eight genome-wide association analyses: Three analyses for eye color (blue versus green, blue versus brown and blue versus non blue), two for hair color (red versus non-red and blond versus brown) and three for skin pigment traits (skin sensitivity to sun, the presence of freckles and a combination of skin sensitivity to sun and presence of freckles herein referred to as “burning and freckling”). These analyses identified 99 distinct SNPs (Table 13) with genome-wide significant associations (P<1.5·10⁻⁷) in at least one of the eight pigmentation scans.

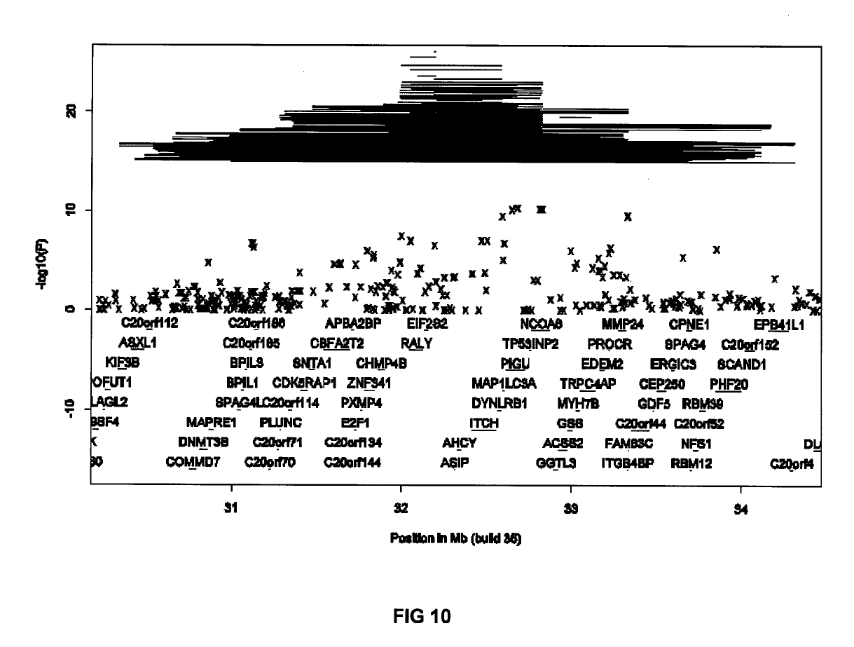

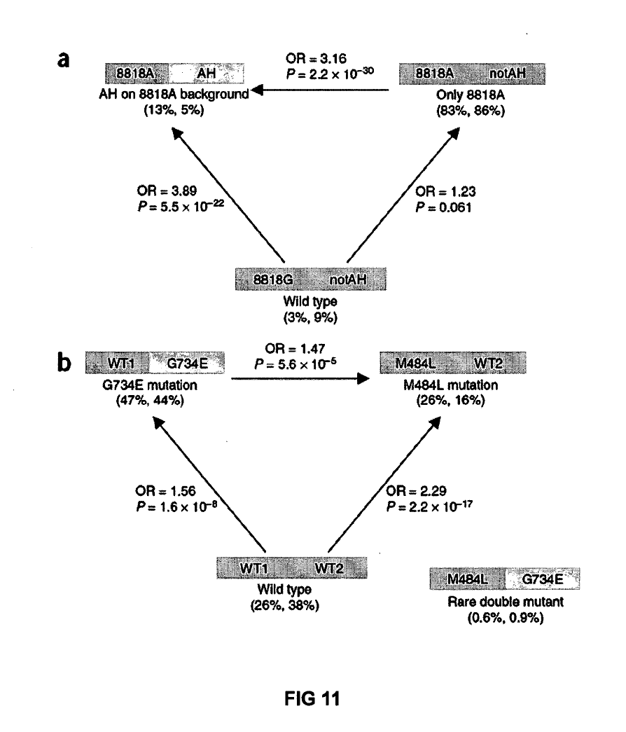

A total of six SNPs within a region of strong linkage disequilibrium (LD) on 20q11.22 showed association with burning and freckling that reached genome-wide significance (max OR=1.60, P=3.9·10⁻⁹, Table 13). Multipoint analysis within the LD area revealed an extended haplotype, tagged by a two SNP haplotype, G rs1015362 T rs4911414, that we will refer to as AH (ASIP Haplotype). The AH haplotype is correlated with the markers rs4911414 and rs1015362, as well as 87 other SNPs in this region (Table 14). However, the AH haplotype accounts for the association of other SNPs in the region (Table 15; FIG. 10) and replicated significantly in both the Icelandic and Dutch replication samples (Table 16). For example, the association of SNP rs910873 , which is correlated with AH (r²=0.71) is weaker than for AH itself (OR 2.73, P-value 2.3×10⁻⁴³ compared with OR 2.99 and P-value 1.4×10⁻⁴⁸ for AH), and the association of rs910873 is not significant when conditioned on AH (OR 1.20, P-value 0.15). In the combined analysis of the discovery and replication samples, AH reached genome-wide significance for red hair color, freckling and skin sensitivity to sun in addition to burning and freckling (Table 16). The region covered by the extended haplotype contains a large number of genes including the well-documented pigment gene ASIP (encoding agouti signaling protein). In melanocytes, the agouti signaling protein antagonizes α-MSH (alpha melanocyte-stimulating hormone) activation of MC1R and results in a switch to the production of red or yellow phaeomelanin. Sequence variants at the agouti locus are responsible for animal coat colors such as yellow and dark color^{3,4}. A polymorphism in the 3′ untranslated region of the ASIP gene, rs6058017 (8818A>G), has been studied for its association with pigmentation characteristics within populations of European ancestry⁵⁻⁷ and has also been related to differences in skin pigmentation among populations of mixed African and European ancestry⁸. The haplotype AH, G rs1015362 T rs4911414, occurs on the background of the major allele of rs6058017 but the correlation between the two is very weak (D′=1; r²=0.008). Consequently, the strength of association of rs6058017 with the pigmentation traits is much less than that of AH, and after adjustment for rs6058017, AH remains highly significant for burning and freckling (P=1.3·10⁻⁴⁶, for burning and freckling). On the other hand, after adjustment for the haplotype, rs6058017 is only marginally associated with the pigmentation characteristics (P=0.057 for burning and freckling; FIG. 11a). Thus, the main association signal in the region is due to AH, which may be the true functional variant. We sequenced the exons and promoter of ASIP in 368 individuals without detecting any sequence variant likely to account for the observed association. A stronger association of AH with skin sensitivity to sun was observed for males than females (P=0.0033), although the difference is not significant after correcting for the number of variants tested for sex specific differences.

Four SNPs on 11q13.2 (FIG. 11) showed association with blond versus brown hair color in the Icelandic discovery sample that reached genome-wide significance (Table 13). The SNPs are located within a single LD block that only overlaps with one gene, TPCN2 (encoding two-pore segment channel 2). Three common non-synonymous mutations in exons of TPCN2 were identified (rs3829241, rs35264875, rs3750965) that, based on the HapMap data, correlate with the four SNPs on the 300K chip giving significant association. These SNPs were typed in the Icelandic discovery samples as well as the two replication samples. The replication samples were also typed with rs1011176 that showed the strongest association in the initial discovery scan. All of the observed association with blond versus brown hair could be explained by two of the coding SNPs: M484L (rs35264875) and G734E (rs3829241) (Table 15) that replicated with similar effects (Table 16). We did not observe strong association of these two variants with the other pigmentation traits (Tables 17-19), similar to what had been observed for the KITLG (encoding the Ligand for KIT receptor tyrosine kinase) variant that also associates with blond versus brown hair. A link between pigmentation and TPCN2 has not been previously suspected. The protein encoded by TPCN2 participates in calcium transport, similarly to the known pigmentation genes SLC24A4¹ and SLC24A5⁹.

A single SNP, rs1408799, on 9p23 showed genome-wide significant association with blue versus non-blue eye (OR=1.41, P=1.5·10⁻⁹). This association was confirmed in both the Icelandic and Dutch replication samples with a similar effect (Table 16). A suggestive association with blond versus brown hair was also observed for this SNP. The SNP belongs to an LD block that encompasses only one gene, TYRP1 (encoding the tyrosinase-related protein 1)¹⁰. TYRP1 encodes a melanosomal enzyme with a role in the eumelanin pathway. In humans, rare mutations in TYRP1 are responsible for oculocutaneous albinism type 3¹¹. Previous studies on the genetics of eye color in Europeans have associated polymorphisms at TYRP1 with eye color¹². The SNP reported here, rs1408799, is in strong LD with one of the previously reported SNPs, rs2733832, in HAPMAP CEU¹³ (D′=0.96; r²=0.67).

The increase in sample size clarify further previously found association signals. For example, the TYR (encoding tyrosinase) mutation rs1126809 (R402Q) reaches genome-wide significance for skin sensitivity to sun in addition to its previously reported association with eye color (Tables 17-19). Compound heterozygotes for a mutant allele of TYR and the R402Q polymorphism can result in ocular albinism¹⁴.

The strength of the association of the new ASIP variant (AH) described here is close to that of variants in the MC1R gene and much stronger than that of the previously reported variants near ASIP. The AH variant is thus likely to be closer to a true functional mutation. It is interesting that the calcium ion transport genes are emerging as a family of pigmentation genes as three have been linked to pigmentation; SLC24A4, SLC24A5, and now TPCN2.

Methods.

The Icelandic Samples.

A total of 5,130 Icelandic adults, recruited through cardiovascular, neoplastic, neurological and metabolic studies, were genotyped for 317,511 SNPs using the HumanHap300 BeadChip (Illumina). These studies were approved by the Data Protection Commission of Iceland and the National Bioethics Committee of Iceland. Written informed consent was obtained from all participants. Personal identifiers associated with phenotypic information and blood samples were encrypted using a third-party encryption system as previously described¹⁵. Only individuals with a genotype yield over 98% were included in the study. A second sample of 2,116 Icelandic individuals was recruited in a similar fashion and genotyped to replicate the SNPs identified in the genome-wide scan.

Each participant completed a questionnaire that included questions about natural eye color categories (blue/gray, green or black/brown), natural hair color categories (red/reddish, blond, dark blond/light brown or brown/black) and the presence of freckles at any time. Skin sensitivity to sun was self-assessed using the Fitzpatrick skin-type score¹⁶, where the lowest score (I) represents very fair skin that is very sensitive to UVR and the highest score (IV) represents dark skin that tans rather than burns in reaction to UVR exposure. Individuals scoring I and II were classified as being sensitive to sun and individuals scoring III and IV were classified as not being sensitive to sun. A combination of skin sensitivity to sun and presence of freckles was performed and referred to as “burning and freckling”.

The Dutch Sample.

The SNPs with the most significant associations that were identified in the genome-wide scans carried out on the Icelandic discovery sample were genotyped and tested for association in a sample of 1,214 Dutch individuals. The Dutch sample was composed of 696 males recruited for a prostate cancer study¹⁷ and 518 females recruited for a breast cancer study¹⁸ by the Radboud University Nijmegen Medical Centre (RUNMC) and through a population-based cancer registry held by the Comprehensive Cancer Centre IKO in Nijmegen. All individuals were of self-reported European ancestry. The study protocol was approved by the Institutional Review Board of Radboud University and all study subjects gave written informed consent for the collection of questionnaire data on lifestyle, medical history and family history.

As in the case of the Icelandic samples, information about pigmentation traits for the Dutch sample was obtained through a questionnaire. The questions about natural eye and hair color were the same as those in the Icelandic questionnaire, with the addition of a category for an ‘other’ eye color. A total of 5.9% of the Dutch participants selected this category and were excluded from our analysis. Skin sensitivity to sun was assessed by two questions about the tendency of individuals to burn or tan when exposed to sun without sun block protection. The answers to these two questions were used to create a dichotomized grouping of individuals according to sensitivity to sun, corresponding to the grouping used for the Icelandic sample. Two questions from the Dutch questionnaire assessed the density of freckles on the face and arms, respectively. For the sake of comparison with the Icelandic data, participants reporting freckles at either location were considered as having freckles present, whereas those reporting absence of freckles at both locations were considered to have no freckles. In addition, the Dutch questionnaire included questions about skin color category (white, white with brownish tint and light-brown), the number of naevi on the left forearm and the number of serious sunburns in their lifetime.

Statistical Methods.

In the genome-wide association stage, Icelandic case and control samples were assayed with the Infinium HumanHap300 SNP chips (Illumina), containing 317,511 SNPs, of which 316,515 were polymorphic and satisfied our quality criteria.

A likelihood procedure described in a previous publication¹⁹ was used for the association analyses. Allele-specific ORs were calculated assuming a multiplicative model²⁰. Results from multiple case-control groups were combined using a Mantel-Haenszel model²¹. In Table 15, 16 and 17, P values for variants at MC1R, TYR, TPCN2 and OCA2 were calculated conditioning for the effect of the other variant at that locus.

Correction for Relatedness and Genomic Control.

Some of the individuals in the Icelandic case-control groups were related to each other, causing the X² test statistic to have a mean >1 and median >0.675². We estimated the inflation factor by using a previously described procedure in which we simulated genotypes through the genealogy of 731,175 Icelanders²². For the initial discovery samples, for which the genotypes for the 316,515 genome-wide SNPs were available, we also estimated the inflation factor by using genomic controls and calculating the average of the 316,515 X² statistics and by computing the median of the 316,515 X² statistics and dividing it by 0.675² as previously described^{23,24}.

Single SNP Genotyping.

SNP genotyping was carried out using the Centaurus (Nanogen) platform²⁵. The quality of each Centaurus SNP assay was evaluated by genotyping each assay in the CEU and/or YRI HapMap samples and comparing the results with the HapMap data. Assays with mismatch rates of >1.5% were not used, and an LD test was used for markers known to be in LD.

Identification of AH

We tested all two marker haplotypes over 264 markers on the Illumina chip in a 4 Mb window around the significant single point association to tanning and burning (FIG. 10). The most signficant association was to the two marker haplotype G rs1015362 T rs4911414. Many other two marker haplotypes in the region tag the same haplotype (e.g. rs2284378 T rs1015362 G and rs4911379 A rs2281695 T). This analyzis localized the association signal to an approximately 1 Mb window between 32 and 33 Mb (in build 36 coordiantes).

### REFERENCES

1. Sulem, P. et al. Genetic determinants of hair, eye and skin pigmentation in Europeans. Nat Genet 39, 1443-52 (2007).
2. Rees, J. L. The genetics of sun sensitivity in humans. Am J Hum Genet 75, 739-51 (2004).
3. Voisey, J. & van Daal, A. Agouti: from mouse to man, from skin to fat. Pigment Cell Res 15, 10-8 (2002).
4. Schmutz, S. M., Berryere, T. G., Barta, J. L., Reddick, K. D. & Schmutz, J. K. Agouti sequence polymorphisms in coyotes, wolves and dogs suggest hybridization. J Hered 98, 351-5 (2007).
5. Kanetsky, P. A. et al. A polymorphism in the agouti signaling protein gene is associated with human pigmentation. Am J Hum Genet 70, 770-5 (2002).
6. Landi, M. T. et al. MC1R, ASIP, and DNA repair in sporadic and familial melanoma in a Mediterranean population. J Natl Cancer Inst 97, 998-1007 (2005).
7. Meziani, R. et al. Association study of the g.8818A>G polymorphism of the human agouti gene with melanoma risk and pigmentary characteristics in a French population. J Dermatol Sci 40, 133-6 (2005).
8. Norton, H. L. et al. Genetic evidence for the convergent evolution of light skin in Europeans and East Asians. Mol Biol Evol 24, 710-22 (2007).
9. Lamason, R. L. et al. SLC24A5, a putative cation exchanger, affects pigmentation in zebrafish and humans. Science 310, 1782-6 (2005).
10. Box, N. F. et al. Complete sequence and polymorphism study of the human TYRP1 gene encoding tyrosinase-related protein 1. Mamm Genome 9, 50-3 (1998).
11. Manga, P. et al. Rufous oculocutaneous albinism in southern African Blacks is caused by mutations in the TYRP1 gene. Am J Hum Genet 61, 1095-101 (1997).
12. Frudakis, T. et al. Sequences associated with human iris pigmentation. Genetics 165, 2071-83 (2003).
13. A haplotype map of the human genome. Nature 437, 1299-320 (2005).
14. Fukai, K. et al. Autosomal recessive ocular albinism associated with a functionally significant tyrosinase gene polymorphism. Nat Genet 9, 92-5 (1995).
15. Gulcher, J. R. Kristjansson, K., Gudbjartsson, H. & Stefansson, K. Protection of privacy by third-party encryption in genetic research in Iceland. Eur J Hum Genet 8, 739-42 (2000).
16. Fitzpatrick, T. B. The validity and practicality of sun-reactive skin types I through VI. Arch Dermatol 124, 869-71 (1988).
17. Gudmundsson, J. et al. Genome-wide association study identifies a second prostate cancer susceptibility variant at 8q24. Nat Genet 39, 631-7 (2007).
18. Stacey, S. N. et al. Common variants on chromosomes 2q35 and 16q12 confer susceptibility to estrogen receptor-positive breast cancer. Nat Genet 39, 865-9 (2007).
19. Gretarsdottir, S. et al. The gene encoding phosphodiesterase 4D confers risk of ischemic stroke. Nat Genet 35, 131-8 (2003).
20. Falk, C. T. & Rubinstein, P. Haplotype relative risks: an easy reliable way to construct a proper control sample for risk calculations. Ann Hum Genet 51 (Pt 3), 227-33 (1987).
21. Mantel, N. & Haenszel, W. Statistical aspects of the analysis of data from retrospective studies of disease. J Natl Cancer Inst. 22, 719-48 (1959).
22. Grant, S. F. et al. Variant of transcription factor 7-like 2 (TCF7L2) gene confers risk of type 2 diabetes. Nat Genet 38, 320-3 (2006).
23. Devlin, B. & Roeder, K. Genomic Control for association studies. Biometrics 55, 997-1004 (1999).
24. Devlin, B., Bacanu, S. -A. & Roeder, K. Genomic control to the extreme. Nature Genetics 36, 1129-1130 (2004).
25. Kutyavin, I. V. et al. A novel endonuclease IV post-PCR genotyping system. Nucleic Acids Research 34, e128 (2006).

TABLE 13
Genome-wide significant SNPs (marker name followed by at-risk associating allele). P
values are corrected using genomic controls.

| | | Build 35 | | | |
|---|---|---|---|---|---|
| SNP | Chr | position | P value | OR | Test |
| rs9378805 C | 6 | 362,727 | 7.7 · 10⁻¹⁰ | 1.32 | freckles present vs. absent |
| rs9328192 G | 6 | 379,364 | 4.9 · 10⁻⁸ | 1.38 | freckles + burns vs. no freckles + tans |
| rs9328192 G | 6 | 379,364 | 7.5 · 10⁻¹⁰ | 1.32 | freckles present vs. absent |
| rs9405681 T | 6 | 394,358 | 5.4 · 10⁻⁸ | 0.765 | freckles present vs. absent |
| rs4959270 C | 6 | 402,748 | 1.2 · 10⁻¹² | 0.655 | freckles + burns vs. no freckles + tans |
| rs4959270 C | 6 | 402,748 | 1.5 · 10⁻¹⁴ | 0.708 | freckles present vs. absent |
| rs1540771 G | 6 | 411,033 | 1.3 · 10⁻¹⁴ | 0.707 | freckles present vs. absent |
| rs1540771 G | 6 | 411,033 | 5.0 · 10⁻¹¹ | 0.676 | freckles + burns vs. no freckles + tans |
| rs1408799 T | 9 | 12,662,097 | 1.5 · 10⁻⁹ | 0.705 | blue vs. green/brown eyes |
| rs896978 T | 11 | 68,585,505 | 3.8 · 10⁻⁸ | 0.617 | blond vs. brown hair |
| rs3750965 G | 11 | 68,596,736 | 7.5 · 10⁻⁹ | 0.607 | blond vs. brown hair |
| rs2305498 T | 11 | 68,623,490 | 5.4 · 10⁻⁸ | 1.63 | blond vs. brown hair |
| rs1011176 G | 11 | 68,690,473 | 6.7 · 10⁻¹⁰ | 0.624 | blond vs. brown hair |
| rs1042602 C | 11 | 88,551,344 | 3.4 · 10⁻⁸ | 1.31 | freckles present vs. absent |
| rs1393350 G | 11 | 88,650,694 | 1.2 · 10⁻¹⁰ | 0.646 | blue vs. green eyes |
| rs1393350 G | 11 | 88,650,694 | 2.7 · 10⁻⁸ | 0.728 | blue vs. green/brown eyes |
| rs12821256 T | 12 | 87,830,803 | 8.4 · 10⁻¹⁸ | 0.468 | blond vs. brown hair |
| rs8016079 G | 14 | 91,828,198 | 4.2 · 10⁻⁸ | 1.62 | blue vs. green eyes |
| rs4904864 G | 14 | 91,834,272 | 1.0 · 10⁻¹⁶ | 2.00 | blond vs. brown hair |
| rs4904864 G | 14 | 91,834,272 | 1.9 · 10⁻¹⁰ | 1.42 | blue vs. green/brown eyes |
| rs4904864 G | 14 | 91,834,272 | 6.1 · 10⁻¹¹ | 1.52 | blue vs. green eyes |
| rs4904868 T | 14 | 91,850,754 | 1.0 · 10⁻²⁰ | 0.481 | blond vs. brown hair |
| rs4904868 T | 14 | 91,850,754 | 2.5 · 10⁻¹⁴ | 0.670 | blue vs. green/brown eyes |
| rs4904868 T | 14 | 91,850,754 | 7.4 · 10⁻¹⁸ | 0.592 | blue vs. green eyes |
| rs2402130 G | 14 | 91,870,956 | 3.2 · 10⁻⁸ | 0.657 | blue vs. green eyes |
| rs2402130 G | 14 | 91,870,956 | 9.4 · 10⁻¹³ | 0.471 | blond vs. brown hair |
| rs1498519 C | 15 | 25,685,246 | 4.0 · 10⁻⁸ | 0.652 | blue vs. brown eyes |
| rs1584407 C | 15 | 25,830,854 | 4.0 · 10⁻¹⁰ | 1.77 | blue vs. brown eyes |
| rs1584407 C | 15 | 25,830,854 | 7.7 · 10⁻⁹ | 1.45 | blue vs. green/brown eyes |
| rs2703952 C | 15 | 25,855,576 | 2.8 · 10⁻¹⁵ | 0.411 | blue vs. brown eyes |
| rs2703952 C | 15 | 25,855,576 | 7.3 · 10⁻¹⁴ | 0.540 | blue vs. green/brown eyes |
| rs2594935 G | 15 | 25,858,633 | 1.2 · 10⁻¹¹ | 1.78 | blue vs. brown eyes |
| rs2594935 G | 15 | 25,858,633 | 1.9 · 10⁻¹⁰ | 1.46 | blue vs. green/brown eyes |
| rs728405 T | 15 | 25,873,448 | 1.6 · 10⁻¹⁶ | 1.71 | blue vs. green/brown eyes |
| rs728405 T | 15 | 25,873,448 | 4.7 · 10⁻¹⁸ | 2.21 | blue vs. brown eyes |
| rs1448488 G | 15 | 25,890,452 | 1.2 · 10⁻¹² | 0.554 | blue vs. brown eyes |
| rs1448488 G | 15 | 25,890,452 | 1.8 · 10⁻¹⁰ | 0.690 | blue vs. green/brown eyes |
| rs4778220 G | 15 | 25,894,733 | 1.8 · 10⁻¹⁰ | 0.617 | blue vs. green/brown eyes |
| rs4778220 G | 15 | 25,894,733 | 4.1 · 10⁻¹⁴ | 0.457 | blue vs. brown eyes |
| rs7170869 G | 15 | 25,962,343 | 1.6 · 10⁻⁸ | 0.548 | blue vs. brown eyes |
| rs7170869 G | 15 | 25,962,343 | 7.6 · 10⁻⁸ | 0.665 | blue vs. green/brown eyes |
| rs11855019 G | 15 | 26,009,415 | 5.6 · 10⁻⁸ | 0.342 | blond vs. brown hair |
| rs11855019 G | 15 | 26,009,415 | 8.6 · 10⁻²⁰ | 0.331 | blue vs. green/brown eyes |
| rs11855019 G | 15 | 26,009,415 | 8.8 · 10⁻³² | 0.175 | blue vs. brown eyes |
| rs6497268 C | 15 | 26,012,308 | 1.1 · 10⁻¹³ | 2.70 | blue vs. green eyes |
| rs6497268 C | 15 | 26,012,308 | 1.9 · 10⁻⁴⁵ | 4.34 | blue vs. green/brown eyes |
| rs6497268 C | 15 | 26,012,308 | 4.0 · 10⁻⁸ | 2.43 | blond vs. brown hair |
| rs6497268 C | 15 | 26,012,308 | 8.4 · 10⁻⁵¹ | 7.31 | blue vs. brown eyes |
| rs7495174 G | 15 | 26,017,833 | 1.5 · 10⁻³⁹ | 0.150 | blue vs. green/brown eyes |
| rs7495174 G | 15 | 26,017,833 | 2.7 · 10⁻⁵⁶ | 0.0733 | blue vs. brown eyes |
| rs7495174 G | 15 | 26,017,833 | 5.0 · 10⁻⁹ | 0.251 | blond vs. brown hair |
| rs7183877 C | 15 | 26,039,328 | 1.5 · 10⁻¹⁰⁷ | 34.5 | blue vs. brown eyes |
| rs7183877 C | 15 | 26,039,328 | 2.5 · 10⁻⁴⁰ | 10.8 | blue vs. green eyes |
| rs7183877 C | 15 | 26,039,328 | 2.8 · 10⁻¹⁵ | 6.27 | blond vs. brown hair |
| rs7183877 C | 15 | 26,039,328 | 2.9 · 10⁻¹⁰³ | 19.4 | blue vs. green/brown eyes |
| rs8028689 T | 15 | 26,162,483 | 3.6 · 10⁻⁵⁸ | 54.2 | blue vs. brown eyes |
| rs8028689 T | 15 | 26,162,483 | 3.9 · 10⁻⁴⁶ | 26.3 | blue vs. green/brown eyes |
| rs8028689 T | 15 | 26,162,483 | 6.0 · 10⁻¹¹ | 9.55 | blue vs. green eyes |
| rs2240204 T | 15 | 26,167,627 | 3.6 · 10⁻⁵⁸ | 0.0184 | blue vs. brown eyes |
| rs2240204 T | 15 | 26,167,627 | 3.9 · 10⁻⁴⁶ | 0.0381 | blue vs. green/brown eyes |
| rs2240204 T | 15 | 26,167,627 | 6.0 · 10⁻¹¹ | 0.105 | blue vs. green eyes |
| rs8039195 T | 15 | 26,189,679 | 3.9 · 10⁻¹²⁹ | 13.1 | blue vs. green/brown eyes |
| rs8039195 T | 15 | 26,189,679 | 4.0 · 10⁻¹⁵⁰ | 26.5 | blue vs. brown eyes |
| rs8039195 T | 15 | 26,189,679 | 6.0 · 10⁻²⁰ | 4.93 | blond vs. brown hair |
| rs8039195 T | 15 | 26,189,679 | 6.3 · 10⁻³⁸ | 6.36 | blue vs. green eyes |
| rs16950979 G | 15 | 26,194,101 | 3.7 · 10⁻⁴⁶ | 0.0381 | blue vs. green/brown eyes |
| rs16950979 G | 15 | 26,194,101 | 3.8 · 10⁻⁵⁸ | 0.0185 | blue vs. brown eyes |
| rs16950979 G | 15 | 26,194,101 | 5.9 · 10⁻¹¹ | 0.105 | blue vs. green eyes |
| rs16950987 G | 15 | 26,199,823 | 3.6 · 10⁻⁵⁸ | 54.2 | blue vs. brown eyes |
| rs16950987 G | 15 | 26,199,823 | 4.0 · 10⁻⁴⁶ | 26.2 | blue vs. green/brown eyes |
| rs16950987 G | 15 | 26,199,823 | 6.1 · 10⁻¹¹ | 9.55 | blue vs. green eyes |
| rs1667394 G | 15 | 26,203,777 | 1.0 · 10⁻⁴³ | 0.147 | blue vs. green eyes |
| rs1667394 G | 15 | 26,203,777 | 1.7 · 10⁻¹⁶¹ | 0.065 | blue vs. green/brown eyes |
| rs1667394 G | 15 | 26,203,777 | 5.2 · 10⁻²⁶ | 0.175 | blond vs. brown hair |
| rs1667394 G | 15 | 26,203,777 | 6.1 · 10⁻¹⁷³ | 0.0295 | blue vs. brown eyes |
| rs1635168 T | 15 | 26,208,861 | 1.4 · 10⁻⁴⁴ | 0.0709 | blue vs. brown eyes |
| rs1635168 T | 15 | 26,208,861 | 9.7 · 10⁻³¹ | 0.147 | blue vs. green/brown eyes |
| rs17137796 T | 15 | 26,798,209 | 1.6 · 10⁻⁸ | 1.47 | blue vs. green/brown eyes |
| rs17137796 T | 15 | 26,798,209 | 6.5 · 10⁻¹⁰ | 1.85 | blue vs. brown eyes |
| rs9932354 C | 16 | 87,580,066 | 6.0 · 10⁻⁸ | 0.629 | red vs. not red hair |
| rs11076747 G | 16 | 87,584,526 | 5.5 · 10⁻¹¹ | 0.566 | red vs. not red hair |
| rs12599126 T | 16 | 87,733,984 | 8.3 · 10⁻⁸ | 1.85 | red vs. not red hair |
| rs9921361 T | 16 | 87,821,940 | 2.3 · 10⁻¹¹ | 4.50 | red vs. not red hair |
| rs4785648 G | 16 | 87,855,978 | 4.9 · 10⁻⁸ | 2.91 | red vs. not red hair |
| rs1466540 T | 16 | 87,871,978 | 9.8 · 10⁻⁹ | 1.74 | red vs. not red hair |
| rs2353028 G | 16 | 87,880,179 | 1.7 · 10⁻¹³ | 0.418 | red vs. not red hair |
| rs2306633 G | 16 | 87,882,779 | 2.2 · 10⁻¹⁶ | 2.85 | red vs. not red hair |
| rs3096304 G | 16 | 87,901,208 | 3.2 · 10⁻⁹ | 0.433 | red vs. not red hair |
| rs2353033 T | 16 | 87,913,062 | 1.2 · 10⁻²⁴ | 0.542 | freckles + burns vs. no freckles + tans |
| rs2353033 T | 16 | 87,913,062 | 2.3 · 10⁻²⁶ | 0.404 | red vs. not red hair |
| rs2353033 T | 16 | 87,913,062 | 3.1 · 10⁻¹² | 0.724 | burns vs. tans |
| rs2353033 T | 16 | 87,913,062 | 5.8 · 10⁻²³ | 0.641 | freckles present vs. absent |
| rs889574 T | 16 | 87,914,309 | 1.5 · 10⁻⁹ | 1.34 | freckles present vs. absent |
| rs889574 T | 16 | 87,914,309 | 8.3 · 10⁻⁹ | 1.44 | freckles + burns vs. no freckles + tans |
| rs2965946 T | 16 | 88,044,113 | 3.9 · 10⁻⁸ | 1.31 | freckles present vs. absent |
| rs4347628 T | 16 | 88,098,136 | 1.7 · 10⁻¹³ | 0.527 | red vs. not red hair |
| rs382745 T | 16 | 88,131,087 | 1.0 · 10⁻¹¹ | 1.84 | red vs. not red hair |
| rs382745 T | 16 | 88,131,087 | 1.0 · 10⁻⁹ | 1.33 | burns vs. tans |
| rs382745 T | 16 | 88,131,087 | 3.9 · 10⁻¹⁸ | 1.70 | freckles + burns vs. no freckles + tans |
| rs382745 T | 16 | 88,131,087 | 9.1 · 10⁻¹⁸ | 1.48 | freckles present vs. absent |
| rs455527 G | 16 | 88,171,502 | 6.1 · 10⁻⁹ | 0.164 | red vs. not red hair |
| rs352935 G | 16 | 88,176,081 | 2.8 · 10⁻¹⁷ | 1.66 | freckles + burns vs. no freckles + tans |
| rs352935 G | 16 | 88,176,081 | 3.9 · 10⁻¹⁴ | 1.92 | red vs. not red hair |
| rs352935 G | 16 | 88,176,081 | 9.1 · 10⁻¹⁹ | 1.49 | freckles present vs. absent |
| rs464349 T | 16 | 88,183,752 | 1.3 · 10⁻²⁰ | 0.658 | freckles present vs. absent |
| rs464349 T | 16 | 88,183,752 | 1.4 · 10⁻¹¹ | 0.557 | red vs. not red hair |
| rs464349 T | 16 | 88,183,752 | 1.8 · 10⁻⁸ | 0.770 | burns vs. tans |
| rs464349 T | 16 | 88,183,752 | 3.7 · 10⁻¹⁹ | 0.585 | freckles + burns vs. no freckles + tans |
| rs164741 T | 16 | 88,219,799 | 1.5 · 10⁻⁶¹ | 4.10 | red vs. not red hair |
| rs164741 T | 16 | 88,219,799 | 1.6 · 10⁻²³ | 1.63 | burns vs. tans |
| rs164741 T | 16 | 88,219,799 | 6.0 · 10⁻⁴⁴ | 2.42 | freckles + burns vs. no freckles + tans |
| rs164741 T | 16 | 88,219,799 | 6.7 · 10⁻³⁸ | 1.86 | freckles present vs. absent |
| rs460879 T | 16 | 88,240,390 | 2.7 · 10⁻³⁸ | 0.314 | red vs. not red hair |
| rs460879 T | 16 | 88,240,390 | 6.0 · 10⁻²⁶ | 0.623 | freckles present vs. absent |
| rs460879 T | 16 | 88,240,390 | 6.4 · 10⁻¹⁴ | 0.708 | burns vs. tans |
| rs460879 T | 16 | 88,240,390 | 9.8 · 10⁻²⁷ | 0.528 | freckles + burns vs. no freckles + tans |
| rs7188458 G | 16 | 88,253,985 | 1.1 · 10⁻⁵⁸ | 0.237 | red vs. not red hair |
| rs7188458 G | 16 | 88,253,985 | 1.4 · 10⁻³⁰ | 0.596 | freckles present vs. absent |
| rs7188458 G | 16 | 88,253,985 | 3.9 · 10⁻²² | 0.640 | burns vs. tans |
| rs7188458 G | 16 | 88,253,985 | 4.6 · 10⁻³⁷ | 0.467 | freckles + burns vs. no freckles + tans |
| rs459920 T | 16 | 88,258,328 | 2.5 · 10⁻²⁴ | 1.84 | freckles + burns vs. no freckles + tans |
| rs459920 T | 16 | 88,258,328 | 4.3 · 10⁻²² | 1.54 | freckles present vs. absent |
| rs459920 T | 16 | 88,258,328 | 5.7 · 10⁻³⁴ | 2.98 | red vs. not red hair |
| rs459920 T | 16 | 88,258,328 | 9.7 · 10⁻¹⁴ | 1.41 | burns vs. tans |
| rs12443954 G | 16 | 88,268,997 | 3.8 · 10⁻²⁴ | 0.206 | red vs. not red hair |
| rs3751700 G | 16 | 88,279,695 | 2.3 · 10⁻⁹ | 3.96 | red vs. not red hair |
| rs258324 C | 16 | 88,281,756 | 2.1 · 10⁻¹¹ | 5.39 | red vs. not red hair |
| rs258322 T | 16 | 88,283,404 | 1.4 · 10⁻²¹ | 1.92 | freckles present vs. absent |
| rs258322 T | 16 | 88,283,404 | 4.2 · 10⁻⁴¹ | 3.84 | red vs. not red hair |
| rs258322 T | 16 | 88,283,404 | 4.2 · 10⁻²⁶ | 2.54 | freckles + burns vs. no freckles + tans |
| rs258322 T | 16 | 88,283,404 | 8.3 · 10⁻¹⁸ | 1.79 | burns vs. tans |
| rs1946482 T | 16 | 88,289,911 | 1.8 · 10⁻⁹ | 2.39 | red vs. not red hair |
| rs3751695 T | 16 | 88,292,050 | 1.3 · 10⁻⁸ | 1.55 | freckles present vs. absent |
| rs3751695 T | 16 | 88,292,050 | 3.8 · 10⁻¹³ | 2.45 | red vs. not red hair |
| rs3751695 T | 16 | 88,292,050 | 8.9 · 10⁻¹² | 1.98 | freckles + burns vs. no freckles + tans |
| rs3751695 T | 16 | 88,292,050 | 9.4 · 10⁻⁸ | 1.52 | burns vs. tans |
| rs6500437 T | 16 | 88,317,399 | 2.2 · 10⁻⁸ | 0.611 | red vs. not red hair |
| rs7204478 T | 16 | 88,322,986 | 1.3 · 10⁻⁶² | 4.44 | red vs. not red hair |
| rs7204478 T | 16 | 88,322,986 | 3.6 · 10⁻²¹ | 1.53 | freckles present vs. absent |
| rs7204478 T | 16 | 88,322,986 | 3.8 · 10⁻²⁶ | 1.88 | freckles + burns vs. no freckles + tans |
| rs7204478 T | 16 | 88,322,986 | 4.6 · 10⁻¹⁵ | 1.44 | burns vs. tans |
| rs1800359 T | 16 | 88,332,762 | 1.2 · 10⁻²⁰ | 0.653 | freckles present vs. absent |
| rs1800359 T | 16 | 88,332,762 | 2.7 · 10⁻¹¹ | 0.729 | burns vs. tans |
| rs1800359 T | 16 | 88,332,762 | 3.7 · 10⁻²² | 0.551 | freckles + burns vs. no freckles + tans |
| rs1800359 T | 16 | 88,332,762 | 4.5 · 10⁻³⁴ | 0.305 | red vs. not red hair |
| rs8058895 T | 16 | 88,342,308 | 2.3 · 10⁻³¹ | 0.349 | red vs. not red hair |
| rs8058895 T | 16 | 88,342,308 | 2.6 · 10⁻¹¹ | 0.690 | burns vs. tans |
| rs8058895 T | 16 | 88,342,308 | 8.1 · 10⁻¹⁴ | 0.663 | freckles present vs. absent |
| rs8058895 T | 16 | 88,342,308 | 9.1 · 10⁻¹⁹ | 0.529 | freckles + burns vs. no freckles + tans |
| rs2011877 C | 16 | 88,342,319 | 5.2 · 10⁻⁸ | 1.61 | red vs. not red hair |
| rs7195066 T | 16 | 88,363,824 | 1.6 · 10⁻¹¹ | 0.638 | freckles + burns vs. no freckles + tans |
| rs7195066 T | 16 | 88,363,824 | 2.1 · 10⁻⁸ | 0.749 | burns vs. tans |
| rs7195066 T | 16 | 88,363,824 | 2.5 · 10⁻⁴³ | 0.179 | red vs. not red hair |
| rs2239359 T | 16 | 88,376,981 | 4.5 · 10⁻¹⁰ | 1.46 | freckles + burns vs. no freckles + tans |
| rs2239359 T | 16 | 88,376,981 | 6.6 · 10⁻⁹ | 1.30 | freckles present vs. absent |
| rs16966142 T | 16 | 88,378,534 | 4.9 · 10⁻¹³ | 0.110 | red vs. not red hair |
| rs1800286 G | 16 | 88,397,262 | 2.2 · 10⁻¹² | 1.39 | burns vs. tans |
| rs1800286 G | 16 | 88,397,262 | 2.8 · 10⁻²³ | 1.84 | freckles + burns vs. no freckles + tans |
| rs1800286 G | 16 | 88,397,262 | 4.8 · 10⁻²¹ | 1.54 | freckles present vs. absent |
| rs1800286 G | 16 | 88,397,262 | 8.8 · 10⁻³⁶ | 3.37 | red vs. not red hair |
| rs11861084 C | 16 | 88,403,211 | 1.6 · 10⁻²² | 1.56 | freckles present vs. absent |
| rs11861084 C | 16 | 88,403,211 | 2.8 · 10⁻¹² | 1.39 | burns vs. tans |
| rs11861084 C | 16 | 88,403,211 | 3.8 · 10⁻²⁴ | 1.86 | freckles + burns vs. no freckles + tans |
| rs11861084 C | 16 | 88,403,211 | 6.0 · 10⁻³⁷ | 3.44 | red vs. not red hair |
| rs8060934 T | 16 | 88,447,526 | 2.9 · 10⁻⁹ | 1.30 | freckles present vs. absent |
| rs8060934 T | 16 | 88,447,526 | 3.5 · 10⁻⁴⁹ | 3.97 | red vs. not red hair |
| rs8060934 T | 16 | 88,447,526 | 3.5 · 10⁻¹² | 1.51 | freckles + burns vs. no freckles + tans |
| rs3803688 T | 16 | 88,462,387 | 5.4 · 10⁻⁸ | 2.81 | red vs. not red hair |
| rs2270460 T | 16 | 88,499,917 | 6.4 · 10⁻¹⁰ | 0.251 | red vs. not red hair |
| rs3212346 G | 16 | 88,509,859 | 4.7 · 10⁻⁹ | 2.91 | red vs. not red hair |
| rs885479 G | 16 | 88,513,655 | 2.1 · 10⁻⁹ | 15.9 | red vs. not red hair |
| rs4785755 G | 16 | 88,565,329 | 1.9 · 10⁻¹⁴ | 1.63 | freckles + burns vs. no freckles + tans |
| rs4785755 G | 16 | 88,565,329 | 2.1 · 10⁻¹¹ | 1.79 | red vs. not red hair |
| rs4785755 G | 16 | 88,565,329 | 2.7 · 10⁻⁸ | 1.32 | burns vs. tans |
| rs4785755 G | 16 | 88,565,329 | 2.7 · 10⁻¹¹ | 1.38 | freckles present vs. absent |
| rs4408545 T | 16 | 88,571,529 | 1.2 · 10⁻³⁶ | 0.565 | freckles present vs. absent |
| rs4408545 T | 16 | 88,571,529 | 1.9 · 10⁻²⁵ | 0.615 | burns vs. tans |
| rs4408545 T | 16 | 88,571,529 | 7.6 · 10⁻⁷² | 0.160 | red vs. not red hair |
| rs4408545 T | 16 | 88,571,529 | 8.9 · 10⁻⁴⁶ | 0.422 | freckles + burns vs. no freckles + tans |
| rs4238833 T | 16 | 88,578,190 | 2.5 · 10⁻³¹ | 0.578 | burns vs. tans |
| rs4238833 T | 16 | 88,578,190 | 3.1 · 10⁻⁴⁷ | 0.513 | freckles present vs. absent |
| rs4238833 T | 16 | 88,578,190 | 3.4 · 10⁻⁸⁴ | 0.178 | red vs. not red hair |
| rs4238833 T | 16 | 88,578,190 | 3.5 · 10⁻⁵⁷ | 0.377 | freckles + burns vs. no freckles + tans |
| rs7201721 G | 16 | 88,586,247 | 8.7 · 10⁻¹⁵ | 1.98 | red vs. not red hair |
| rs4785763 C | 16 | 88,594,437 | 1.8 · 10⁻⁴⁶ | 0.512 | freckles present vs. absent |
| rs4785763 C | 16 | 88,594,437 | 3.0 · 10⁻⁸⁶ | 0.178 | red vs. not red hair |
| rs4785763 C | 16 | 88,594,437 | 3.1 · 10⁻⁵⁷ | 0.375 | freckles + burns vs. no freckles + tans |
| rs4785763 C | 16 | 88,594,437 | 8.2 · 10⁻³² | 0.573 | burns vs. tans |
| rs9936896 T | 16 | 88,596,560 | 2.8 · 10⁻¹⁸ | 0.627 | freckles present vs. absent |
| rs9936896 T | 16 | 88,596,560 | 3.7 · 10⁻¹⁴ | 0.665 | burns vs. tans |
| rs9936896 T | 16 | 88,596,560 | 4.6 · 10⁻²⁴ | 0.493 | freckles + burns vs. no freckles + tans |
| rs9936896 T | 16 | 88,596,560 | 6.9 · 10⁻²⁰ | 0.439 | red vs. not red hair |
| rs8059973 G | 16 | 88,607,035 | 2.9 · 10⁻⁹ | 2.50 | red vs. not red hair |
| rs11648785 T | 16 | 88,612,062 | 1.3 · 10⁻²³ | 0.616 | freckles present vs. absent |
| rs11648785 T | 16 | 88,612,062 | 3.7 · 10⁻²⁷ | 0.494 | freckles + burns vs. no freckles + tans |
| rs11648785 T | 16 | 88,612,062 | 5.2 · 10⁻²³ | 0.355 | red vs. not red hair |
| rs11648785 T | 16 | 88,612,062 | 5.3 · 10⁻¹⁴ | 0.685 | burns vs. tans |
| rs2241039 T | 16 | 88,615,938 | 1.2 · 10⁻¹³ | 0.700 | burns vs. tans |
| rs2241039 T | 16 | 88,615,938 | 1.3 · 10⁻²⁹ | 0.495 | freckles + burns vs. no freckles + tans |
| rs2241039 T | 16 | 88,615,938 | 3.2 · 10⁻³³ | 0.300 | red vs. not red hair |
| rs2241039 T | 16 | 88,615,938 | 3.7 · 10⁻²⁸ | 0.600 | freckles present vs. absent |
| rs3785181 G | 16 | 88,632,834 | 9.5 · 10⁻¹¹ | 6.43 | red vs. not red hair |
| rs1048149 T | 16 | 88,638,451 | 1.2 · 10⁻⁹ | 1.39 | burns vs. tans |
| rs1048149 T | 16 | 88,638,451 | 1.4 · 10⁻¹⁵ | 2.07 | red vs. not red hair |
| rs1048149 T | 16 | 88,638,451 | 2.1 · 10⁻¹⁵ | 1.74 | freckles + burns vs. no freckles + tans |
| rs1048149 T | 16 | 88,638,451 | 4.2 · 10⁻¹⁰ | 1.40 | freckles present vs. absent |
| rs4785612 C | 16 | 88,640,608 | 1.2 · 10⁻¹² | 1.61 | freckles + burns vs. no freckles + tans |
| rs4785612 C | 16 | 88,640,608 | 3.2 · 10⁻⁹ | 1.70 | red vs. not red hair |
| rs4785612 C | 16 | 88,640,608 | 3.3 · 10⁻¹⁰ | 1.38 | freckles present vs. absent |
| rs2078478 T | 16 | 88,657,637 | 6.2 · 10⁻⁹ | 0.378 | red vs. not red hair |
| rs7196459 T | 16 | 88,668,978 | 1.6 · 10⁻³⁴ | 3.35 | red vs. not red hair |
| rs7196459 T | 16 | 88,668,978 | 4.5 · 10⁻²⁵ | 1.96 | freckles present vs. absent |
| rs7196459 T | 16 | 88,668,978 | 6.8 · 10⁻²⁰ | 1.80 | burns vs. tans |
| rs7196459 T | 16 | 88,668,978 | 8.3 · 10⁻³⁴ | 2.78 | freckles + burns vs. no freckles + tans |
| rs2281695 T | 20 | 32,592,825 | 1.8 · 10⁻⁸ | 1.49 | freckles + burns vs. no freckles + tans |
| rs2378199 T | 20 | 32,650,141 | 5.2 · 10⁻⁹ | 1.59 | freckles + burns vs. no freckles + tans |
| rs2378249 G | 20 | 32,681,751 | 3.9 · 10⁻⁹ | 1.60 | freckles + burns vs. no freckles + tans |
| rs6060034 T | 20 | 32,815,525 | 4.9 · 10⁻⁹ | 1.59 | freckles + burns vs. no freckles + tans |
| rs6060043 T | 20 | 32,828,245 | 4.5 · 10⁻⁹ | 0.628 | freckles + burns vs. no freckles + tans |
| rs619865 G | 20 | 33,331,111 | 1.6 · 10⁻⁸ | 0.619 | freckles + burns vs. no freckles + tans |

TABLE 14
Surrogate markers in LD with the AH haplotype (G rs1015362
T rs4911414).

| | | | | | Seq |
|---|---|---|---|---|---|
| SNP | Pos Build 36 | p-value | R2 | D′ | ID No: |
| rs1885120 | 33040650 | 4.29E−13 | 0.880952 | 1 | 139 |
| rs17401449 | 31531606 | 5.08E−10 | 0.73684 | 1 | 140 |
| rs291671 | 31414506 | 5.08E−10 | 0.73684 | 1 | 141 |
| rs291695 | 31451863 | 5.08E−10 | 0.73684 | 1 | 142 |
| rs293721 | 31493100 | 5.08E−10 | 0.73684 | 1 | 143 |
| rs721970 | 31367194 | 5.08E−10 | 0.73684 | 1 | 144 |
| rs910873 | 32635433 | 1.25E−11 | 0.707792 | 1 | 145 |
| rs17305573 | 32643813 | 1.39E−11 | 0.704852 | 1 | 146 |
| rs4911442 | 32818707 | 2.99E−11 | 0.683413 | 1 | 147 |
| rs1204552 | 34102317 | 3.08E−09 | 0.58591 | 0.863452 | 148 |
| rs293709 | 31401767 | 0.000129 | 0.482026 | 1 | 149 |
| rs6058091 | 32662051 | 1.62E−09 | 0.460497 | 1 | 150 |
| rs1884431 | 32802246 | 2.75E−09 | 0.442507 | 1 | 151 |
| rs6142199 | 32625959 | 4.56E−09 | 0.415684 | 1 | 152 |
| rs2068474 | 32694740 | 5.36E−09 | 0.404762 | 1 | 153 |
| rs2378199 | 32650141 | 5.36E−09 | 0.404762 | 1 | 47 |
| rs2378249 | 32681751 | 5.36E−09 | 0.404762 | 1 | 48 |
| rs2425003 | 32867245 | 5.36E−09 | 0.404762 | 1 | 154 |
| rs4302281 | 32635306 | 5.36E−09 | 0.404762 | 1 | 155 |
| rs4564863 | 32643028 | 5.36E−09 | 0.404762 | 1 | 156 |
| rs4911430 | 32609065 | 5.36E−09 | 0.404762 | 1 | 157 |
| rs6059928 | 32631010 | 5.36E−09 | 0.404762 | 1 | 158 |
| rs6059937 | 32649861 | 5.36E−09 | 0.404762 | 1 | 159 |
| rs6059961 | 32695151 | 5.36E−09 | 0.404762 | 1 | 160 |
| rs6059969 | 32708945 | 5.36E−09 | 0.404762 | 1 | 161 |
| rs6087607 | 32661150 | 5.36E−09 | 0.404762 | 1 | 162 |
| rs2144956 | 32609529 | 5.74E−09 | 0.402526 | 1 | 163 |
| rs2295443 | 32637488 | 5.74E−09 | 0.402526 | 1 | 164 |
| rs2889849 | 32627938 | 5.74E−09 | 0.402526 | 1 | 165 |
| rs6058089 | 32657918 | 5.74E−09 | 0.402526 | 1 | 166 |
| rs6059916 | 32612522 | 5.74E−09 | 0.402526 | 1 | 167 |
| rs932542 | 32635029 | 5.74E−09 | 0.402526 | 1 | 168 |
| rs17421899 | 33398852 | 5.17E−07 | 0.395943 | 0.855529 | 169 |
| rs1884432 | 32806100 | 8.84E−09 | 0.387056 | 1 | 170 |
| rs7265992 | 32989068 | 1.06E−08 | 0.381089 | 1 | 171 |
| rs17092148 | 32898822 | 9.61E−09 | 0.379699 | 1 | 172 |
| rs3787220 | 32801412 | 9.61E−09 | 0.379699 | 1 | 173 |
| rs3787223 | 32795046 | 9.61E−09 | 0.379699 | 1 | 174 |
| rs6058115 | 32822058 | 9.61E−09 | 0.379699 | 1 | 175 |
| rs6060009 | 32767635 | 9.61E−09 | 0.379699 | 1 | 176 |
| rs6060017 | 32776703 | 9.61E−09 | 0.379699 | 1 | 177 |
| rs6060030 | 32803974 | 9.61E−09 | 0.379699 | 1 | 178 |
| rs6060034 | 32815525 | 9.61E−09 | 0.379699 | 1 | 89 |
| rs6060043 | 32828245 | 9.61E−09 | 0.379699 | 1 | 90 |
| rs6060047 | 32831061 | 9.61E−09 | 0.379699 | 1 | 179 |
| rs6088594 | 32806818 | 9.61E−09 | 0.379699 | 1 | 180 |
| rs7271289 | 32860964 | 9.61E−09 | 0.379699 | 1 | 181 |
| rs910871 | 32796869 | 9.61E−09 | 0.379699 | 1 | 182 |
| rs6088316 | 31890503 | 1.03E−08 | 0.377279 | 1 | 183 |
| rs17396317 | 31254038 | 2.37E−07 | 0.367249 | 0.856934 | 184 |
| rs2425067 | 33671930 | 9.49E−07 | 0.364667 | 0.853898 | 185 |
| rs6058339 | 33923893 | 9.49E−07 | 0.364667 | 0.853898 | 186 |
| rs6060612 | 33853941 | 9.49E−07 | 0.364667 | 0.853898 | 187 |
| rs2378412 | 33939716 | 1.07E−06 | 0.360001 | 0.853613 | 188 |
| rs293738 | 31389579 | 4.37E−07 | 0.340008 | 0.855757 | 189 |
| rs1205339 | 32388628 | 2.75E−08 | 0.336735 | 1 | 190 |
| rs2281695 | 32592825 | 2.75E−08 | 0.336735 | 1 | 41 |
| rs4911154 | 32459762 | 2.75E−08 | 0.336735 | 1 | 191 |
| rs6088515 | 32573703 | 2.75E−08 | 0.336735 | 1 | 192 |
| rs7269526 | 32516954 | 2.75E−08 | 0.336735 | 1 | 193 |
| rs17305657 | 31270249 | 2.49E−06 | 0.335292 | 0.722188 | 194 |
| rs1122174 | 32574507 | 3.14E−08 | 0.332661 | 1 | 195 |
| rs6060025 | 32790537 | 3.28E−08 | 0.331225 | 1 | 196 |
| rs6059908 | 32595820 | 4.45E−08 | 0.318182 | 1 | 197 |
| rs4911523 | 34008909 | 0.000381 | 0.31732 | 1 | 198 |
| rs4911315 | 31349907 | 4.83E−06 | 0.304429 | 0.719584 | 199 |
| rs619865 | 33331111 | 2.04E−05 | 0.298956 | 0.712909 | 92 |
| rs6059931 | 32638999 | 1.27E−06 | 0.294437 | 0.852863 | 200 |
| rs11546155 | 32914809 | 1.27E−06 | 0.29441 | 0.852792 | 201 |
| rs221981 | 31112517 | 0.000644 | 0.288926 | 0.567439 | 202 |
| rs17122844 | 32916261 | 1.26E−07 | 0.283623 | 1 | 203 |
| rs7272741 | 31129541 | 6.51E−05 | 0.276109 | 0.681442 | 204 |
| rs2425020 | 33287248 | 4.67E−07 | 0.237792 | 1 | 205 |
| rs2424941 | 31128943 | 4E−05 | 0.237395 | 0.711465 | 206 |
| rs761930 | 31151255 | 5.14E−05 | 0.231719 | 0.706448 | 207 |
| rs221984 | 31133178 | 0.00035 | 0.226858 | 0.668736 | 208 |
| rs2378078 | 32178389 | 6.82E−07 | 0.224967 | 1 | 209 |
| rs2424944 | 31135289 | 0.000675 | 0.224581 | 0.663143 | 210 |
| rs633784 | 33189984 | 8.76E−07 | 0.217714 | 1 | 211 |
| rs666210 | 33187471 | 8.76E−07 | 0.217714 | 1 | 212 |
| rs7361656 | 33192808 | 8.76E−07 | 0.217714 | 1 | 213 |
| rs2424948 | 31136117 | 0.000153 | 0.214027 | 0.702035 | 214 |
| rs2424994 | 32596578 | 1.14E−05 | 0.213533 | 0.845135 | 215 |
| rs221985 | 31133205 | 0.000512 | 0.211507 | 0.664664 | 216 |
| rs17092378 | 33199849 | 1.20E−06 | 0.2084 | 1 | 217 |
| rs2050652 | 33196841 | 1.31E−06 | 0.206241 | 1 | 218 |
| rs6058192 | 33197922 | 1.31E−06 | 0.206241 | 1 | 219 |
| rs6059662 | 32139388 | 1.30E−06 | 0.205752 | 1 | 220 |
| rs7274811 | 31796842 | 3.38E−05 | 0.201156 | 0.838959 | 221 |

Surrogate markers were selected based on HapMap CEU in a 4 megabase interval flanking the haplotype.
Shown is surrogate marker name, its position in NCBI Build 36, and the P-value, r2 and D′ of the surrogate with the AH haplotype.

TABLE 15
Refinement of signals at the ASIP, TPCN2 and TYR loci. The four
variants shown at the ASIP locus are: The ASIP haplotype tagged by
rs1015362 G rs4911414 T (^{a}AH), the previously studied
g.8818A > G (^{b}rs6058017 A)^{3,4}, and a SNP
showing significant association in the originial genome-wide
association scan (rs6060043 T). The four variants shown at the
TPCN2 locus are: The SNP showing the most significant association
signal in the genome-wide association scan (rs1011176 A) and
three missense mutations SNPs in TPCN2 (rs3829241 G, rs35264875 T,
rs3750965 A).

| | Iceland | | The Netherlands | | |
|---|---|---|---|---|---|
| Variant | OR | P | OR | P | Combined P |

ASIP
Marginal test for association of variants at the ASIP locus with burning
and freckling

| | | | | | |
|---|---|---|---|---|---|
| AH^{a} | 2.99 | 1.8 · 10⁻⁴⁴ | 2.29 | 5.6 · 10⁻⁶ | 1.4 · 10⁻⁴⁸ |
| rs6058017 A^{b} | 1.54 | 1.6 · 10⁻⁵ | 0.91 | 0.59 | 6.0 · 10⁻⁴ |
| rs6060043 T | 1.79 | 5.8 · 10⁻²⁵ | 1.49 | 0.0028 | 1.4 · 10⁻²⁶ |

Test for association of variants at the ASIP locus with burning and
freckling, conditional on the effect of AH^{a}

| | | | | | |
|---|---|---|---|---|---|
| rs6058017 A | 1.34 | 0.0044 | 0.83 | 0.28 | 0.057 |
| rs6060043 T | 0.88 | 0.097 | 0.93 | 0.65 | 0.088 |

TPCN2
Marginal test for association of variants at the TPCN2 locus with blonde
vs. brown hair

| | | | | | |
|---|---|---|---|---|---|
| rs3829241 G | 1.23 | 0.0017 | 1.16 | 0.22 | 0.00085 |
| rs1011176 A | 1.63 | 2.1 · 10⁻¹⁴ | 1.46 | 0.002 | 2.4 · 10⁻¹⁶ |
| rs35264875 T | 1.89 | 1.7 · 10⁻¹¹ | 1.78 | 0.00021 | 1.6 · 10⁻¹⁴ |
| rs3750965 A | 1.63 | 1.6 · 10⁻¹¹ | 1.22 | 0.11 | 3.0 · 10⁻¹¹ |

Test for association of variant at the TPCN2 locus with blonde vs. brown
hair, conditional on the effect of rs35264875

| | | | | | |
|---|---|---|---|---|---|
| rs3829241 G | 1.57 | 8.0 · 10⁻¹⁰ | 1.38 | 0.012 | 4.8 · 10⁻¹¹ |
| rs1011176 A | 1.47 | 6.0 · 10⁻⁸ | 1.34 | 0.021 | 4.9 · 10⁻⁹ |
| rs3750965 A | 1.43 | 4.3 · 10⁻⁶ | 1.07 | 0.62 | 2.4 · 10⁻⁵ |

Test for association of variant at the TPCN2 locus with blonde vs. brown
hair, conditional on the effects of rs35264875 and rs3829241

| | | | | | |
|---|---|---|---|---|---|
| rs1011176 A | 1.14 | 0.22 | 1.18 | 0.29 | 0.11 |
| rs3750965 A | 0.91 | 0.49 | 0.69 | 0.057 | 0.094 |

TABLE 16
Association of SNPs in TPCN2 and TYRP and the AH haplotype in ASIP to pigmentation
characteristics in Iceland and the Netherlands. ORs an their 95% confidence intervals are given
for each sample. See Tables 17-19 for association to other pigmentation traits.

| | OR (95% CI) | | | |
|---|---|---|---|---|
| | Iceland | Iceland | Netherland | |
| Locus | Discovery | Replication | Replication | |
| Phenotype | (N = 5,130) | (N = 2,116) | (N = 1,214) | P |

ASIP AH (rs1015362 G rs4911414 T) (freq 8%)

| | | | | |
|---|---|---|---|---|
| Burn and freckle^{a} | 2.56 (2.06, 3.18) | 2.90 (2.11, 3.98) | 2.27 (1.58, 3.26) | 5.8 · 10⁻³⁷ |
| Skin sensitivity to sun^{b} | 1.76 (1.49, 2.08) | 1.82 (1.43, 2.32) | 1.75 (1.32, 2.32) | 1.9 · 10⁻²⁴ |
| Freckle^{c} | 1.95 (1.65, 2.32) | 2.13 (1.66, 2.72) | 1.56 (1.17, 2.07) | 8.2 · 10⁻²⁹ |
| Red vs. not red hair | 1.76 (1.34, 2.31) | 2.02 (1.38, 2.96) | 2.03 (0.93, 4.46) | 2.7 · 10⁻⁹ |
| Blond vs. brown hair | 1.46 (1.08, 1.96) | 1.62 (1.08, 2.43) | 1.75 (1.15, 2.66) | 1.5 · 10⁻⁵ |

TPCN2 rs35264875 T (freq 22%)^{d}

| | | | | |
|---|---|---|---|---|
| Blond vs. brown hair | 2.49 (1.96, 3.15) | 2.13 (1.38, 3.30) | 2.03 (1.47, 2.80) | 3.6 · 10⁻³⁰ |

TPCN2 rs3829241 A (freq 44%)^{d}

| | | | | |
|---|---|---|---|---|
| Blond vs. brown hair | 1.60 (1.35, 1.89) | 1.54 (1.12, 2.11) | 1.38 (1.07, 1.77) | 6.2 · 10⁻¹⁶ |

TYRP1 rs1408799 C (freq 75%)

| | | | | |
|---|---|---|---|---|
| Blue vs. green/brown eyes | 1.40 (1.25, 1.57) | 1.32 (1.11, 1.58) | 1.22 (1.01, 1.47) | 5.9 · 10⁻¹⁷ |
| Blond vs. brown hair | 1.29 (1.09, 1.53) | 1.10 (0.85, 1.42) | 1.10 (0.86, 1.42) | 8.3 · 10⁻⁵ |

^{a}Compared to those who tan and do not freckle.
^{b}Compared to those who are not sensitive to sun.
^{c}Compared to those who do not freckle.
^{d}The effects of the two TPCN2 SNPs were estimated jointly.

TABLE 17
Association analysis of eye colour in 5,130 Icelandic discovery individuals, 2,116
Icelandic replication individuals and 1,214 Dutch replication individuals.

| | | | Iceland | | | |
|---|---|---|---|---|---|---|
| | | | Discovery | Replication | Netherlands | |
| | Locus | Variant | OR (95% c.i.) | OR (95% c.i.) | OR (95% c.i.) | P |
| Blue vs. brown | SLC24A4 | rs12896399 T | 1.25 (1.07, 1.46) | 1.35 (1.04, 1.74) | 1.11 (0.91, 1.36) | 0.00011 |
| | KITLG | rs12821256 C | 1.06 (0.88, 1.28) | 1.13 (0.83, 1.55) | 0.97 (0.72, 1.31) | 0.51 |
| | 6P25.3 | rs1540771 A | 1.09 (0.94, 1.27) | 1.21 (0.94, 1.57) | 1.07 (0.87, 1.30) | 0.11 |
| | TYR | rs1126809 A | 1.16 (0.98, 1.37) | 1.17 (0.89, 1.54) | 1.25 (1.00, 1.56) | 0.002 |
| | | rs1042602 C | 0.93 (0.79, 1.10) | 1.01 (0.76, 1.34) | 0.98 (0.80, 1.20) | 0.37 |
| | OCA2 | rs1667394 A | 28.00 (21.83, 35.91) | 19.01 (12.38, 29.18) | 15.38 (10.78, 21.94) | <10⁻³⁰⁰ |
| | | rs7495174 A | 5.81 (3.62, 9.30) | 5.34 (2.51, 11.39) | 4.98 (2.49, 9.95) | 1.5 · 10⁻²⁹ |
| | MC1R | rs1805008 T | 1.16 (0.92, 1.46) | 0.96 (0.65, 1.42) | 1.28 (0.88, 1.88) | 0.086 |
| | | rs1805007 T | 1.12 (0.86, 1.47) | 0.80 (0.54, 1.19) | 0.92 (0.61, 1.39) | 0.43 |
| | TPCN2 | rs35264875 T | 1.05 (0.82, 1.35) | 1.32 (0.73, 2.39) | 0.97 (0.74, 1.28) | 0.86 |
| | | rs3829241 A | 0.95 (0.80, 1.13) | 1.12 (0.76, 1.64) | 1.14 (0.92, 1.42) | 0.91 |
| | ASIP | AH^{a} | 0.92 (0.69, 1.21) | 1.47 (0.90, 2.41) | 1.02 (0.71, 1.47) | 0.62 |
| | TYRP1 | rs1408799 T | 1.40 (1.18, 1.65) | 1.49 (1.12, 1.98) | 1.11 (0.89, 1.38) | 1.9 · 10⁻⁷ |
| Blue vs. green eyes | SLC24A4 | rs12896399 T | 1.93 (1.71, 2.18) | 1.53 (1.28, 1.83) | 2.03 (1.54, 2.66) | 1.5 · 10⁻⁵² |
| | KITLG | rs12821256 C | 1.01 (0.87, 1.16) | 1.21 (0.97, 1.51) | 1.19 (0.78, 1.81) | 0.73 |
| | 6P25.3 | rs1540771 A | 0.98 (0.87, 1.11) | 1.13 (0.95, 1.35) | 0.88 (0.68, 1.15) | 0.51 |
| | TYR | rs1126809 A | 1.56 (1.36, 1.78) | 1.47 (1.21, 1.79) | 1.49 (1.10, 2.01) | 4.6 · 10⁻²¹ |
| | | rs1042602 C | 0.97 (0.86, 1.11) | 0.97 (0.80, 1.18) | 1.17 (0.89, 1.53) | 0.88 |
| | OCA2 | rs1667394 A | 6.57 (4.97, 8.68) | 5.48 (3.60, 8.33) | 5.92 (3.46, 10.14) | 3.0 · 10⁻⁸⁷ |
| | | rs7495174 A | 1.47 (0.93, 2.32) | 2.04 (1.02, 4.06) | 1.46 (0.53, 4.03) | 0.018 |
| | MC1R | rs1805008 T | 1.00 (0.80, 1.25) | 0.79 (0.61, 1.03) | 0.87 (0.55, 1.38) | 0.83 |
| | | rs1805007 T | 0.86 (0.70, 1.05) | 0.68 (0.52, 0.89) | 1.10 (0.62, 1.95) | 0.091 |
| | TPCN2 | rs35264875 T | 1.18 (0.97, 1.45) | 0.93 (0.67, 1.31) | 0.90 (0.64, 1.28) | 0.048 |
| | | rs3829241 A | 1.01 (0.89, 1.15) | 1.07 (0.83, 1.37) | 1.18 (0.89, 1.56) | 0.52 |
| | ASIP | AH^{a} | 0.77 (0.63, 0.96) | 0.83 (0.62, 1.12) | 0.92 (0.58, 1.48) | 0.0010 |
| | TYRP1 | rs1408799 T | 1.40 (1.23, 1.60) | 1.25 (1.02, 1.53) | 1.47 (1.11, 1.95) | 1.6 · 10⁻¹³ |
| Blue vs. green or | SLC24A4 | rs12896399 T | 1.62 (1.46, 1.80) | 1.47 (1.25, 1.72) | 1.34 (1.13, 1.60) | 6.4 · 10⁻³⁹ |
| brown eyes | KITLG | rs12821256 C | 1.03 (0.91, 1.16) | 1.19 (0.97, 1.44) | 1.04 (0.79, 1.35) | 0.57 |
| | 6P25.3 | rs1540771 A | 1.02 (0.93, 1.13) | 1.16 (0.99, 1.36) | 1.00 (0.84, 1.19) | 0.64 |
| | TYR | rs1126809 A | 1.38 (1.23, 1.55) | 1.36 (1.14, 1.62) | 1.32 (1.09, 1.60) | 8.7 · 10⁻¹⁷ |
| | | rs1042602 C | 0.96 (0.86, 1.07) | 0.98 (0.82, 1.17) | 1.04 (0.87, 1.24) | 0.38 |
| | OCA2 | rs1667394 A | 13.38 (10.85, 16.48) | 9.21 (6.58, 12.89) | 11.62 (8.36, 16.15) | <10⁻³⁰⁰ |
| | | rs7495174 A | 2.78 (1.93, 3.99) | 3.36 (1.87, 6.06) | 4.22 (2.19, 8.10) | 8.9 · 10⁻¹⁹ |
| | MC1R | rs1805008 T | 1.06 (0.91, 1.24) | 0.84 (0.66, 1.06) | 1.11 (0.81, 1.53) | 0.26 |
| | | rs1805007 T | 0.95 (0.80, 1.14) | 0.72 (0.56, 0.91) | 0.97 (0.68, 1.40) | 0.53 |
| | TPCN2 | rs35264875 T | 1.13 (0.95, 1.34) | 1.02 (0.73, 1.43) | 0.95 (0.75, 1.20) | 0.12 |
| | | rs3829241 A | 0.99 (0.88, 1.11) | 1.09 (0.87, 1.37) | 1.15 (0.96, 1.39) | 0.55 |
| | ASIP | AH^{a} | 0.82 (0.69, 0.99) | 0.98 (0.74, 1.29) | 0.99 (0.73, 1.35) | 0.023 |
| | TYRP1 | rs1408799 T | 1.40 (1.25, 1.57) | 1.32 (1.11, 1.58) | 1.22 (1.01, 1.47) | 5.9 · 10⁻¹⁷ |

^{a}AH is the haplotype G rs1015362 T rs4911414.

TABLE 18
Association of genetic variants to hair colour in 5,130 Icelandic discovery individuals,
2,116 Icelandic replication individuals and 1,214 Dutch replication individuals.

| | | | Iceland | | | |
|---|---|---|---|---|---|---|
| | | | Discovery | Replication | Netherlands | |
| | Locus | Variant | OR (95% c.i.) | OR (95% c.i.) | OR (95% c.i.) | P |
| Red hair | SLC24A4 | rs12896399 T | 0.95 (0.81, 1.12) | 0.98 (0.76, 1.27) | 0.89 (0.53, 1.49) | 0.42 |
| | KITLG | rs12821256 C | 0.96 (0.79, 1.18) | 0.91 (0.66, 1.26) | 0.65 (0.27, 1.54) | 0.51 |
| | 6P25.3 | rs1540771 A | 1.06 (0.90, 1.25) | 1.12 (0.87, 1.45) | 1.05 (0.63, 1.76) | 0.32 |
| | TYR | rs1126809 A | 0.98 (0.83, 1.17) | 1.16 (0.89, 1.53) | 0.83 (0.47, 1.49) | 0.70 |
| | | rs1042602 C | 0.83 (0.70, 0.99) | 0.97 (0.73, 1.29) | 1.26 (0.74, 2.15) | 0.025 |
| | OCA2 | rs1667394 A | 0.89 (0.63, 1.27) | 0.76 (0.44, 1.33) | 1.44 (0.52, 3.95) | 0.57 |
| | | rs7495174 A | 1.48 (0.82, 2.68) | 1.05 (0.44, 2.50) | 1.16 (0.23, 5.79) | 0.14 |
| | MC1R | rs1805008 T | 8.73 (6.97, 10.94) | 10.20 (7.23, 14.40) | 7.71 (3.53, 16.83) | 1.4 · 10⁻¹⁶² |
| | | rs1805007 T | 14.09 (11.17, 17.77) | 13.33 (9.49, 18.73) | 20.32 (10.47, 39.43) | 8.8 · 10⁻²³⁶ |
| | TPCN2 | rs35264875 T | 0.99 (0.82, 1.20) | 1.22 (0.75, 1.96) | 1.60 (0.84, 3.02) | 0.73 |
| | | rs3829241 A | 0.94 (0.79, 1.13) | 0.84 (0.60, 1.17) | 0.92 (0.52, 1.62) | 0.41 |
| | ASIP | AH^{a} | 1.76 (1.34, 2.31) | 2.02 (1.38, 2.96) | 1.98 (0.91, 4.32) | 3.0 · 10⁻⁹ |
| | TYRP1 | rs1408799 T | 0.92 (0.76, 1.11) | 1.04 (0.77, 1.40) | 1.36 (0.77, 2.43) | 0.29 |
| Blonde vs. brown hair | SLC24A4 | rs12896399 T | 2.55 (2.19, 2.97) | 2.35 (1.88, 2.94) | 1.88 (1.49, 2.38) | 1.9 · 10⁻⁷⁰ |
| | KITLG | rs12821256 C | 2.14 (1.79, 2.54) | 1.99 (1.52, 2.60) | 2.45 (1.68, 3.57) | 3.1 · 10⁻³⁸ |
| | 6P25.3 | rs1540771 A | 0.70 (0.60, 0.80) | 0.79 (0.63, 0.98) | 0.93 (0.73, 1.17) | 2.1 · 10⁻¹¹ |
| | TYR | rs1126809 A | 1.25 (1.06, 1.46) | 1.44 (1.14, 1.83) | 1.29 (1.00, 1.67) | 2.7 · 10⁻⁵ |
| | | rs1042602 C | 0.83 (0.71, 0.97) | 0.82 (0.64, 1.04) | 0.94 (0.74, 1.20) | 0.0011 |
| | OCA2 | rs1667394 A | 5.06 (3.57, 7.18) | 6.78 (3.76, 12.20) | 5.53 (3.51, 8.72) | 2.4 · 10⁻⁴⁹ |
| | | rs7495174 A | 1.83 (1.05, 3.20) | 1.91 (0.70, 5.18) | 0.83 (0.41, 1.71) | 0.018 |
| | MC1R | rs1805008 T | 1.89 (1.49, 2.38) | 1.97 (1.39, 2.80) | 1.98 (1.29, 3.04) | 3.9 · 10⁻¹⁶ |
| | | rs1805007 T | 2.08 (1.59, 2.73) | 2.21 (1.55, 3.14) | 1.68 (1.00, 2.82) | 1.2 · 10⁻¹⁴ |
| | TPCN2 | rs35264875 T | 2.49 (1.96, 3.15) | 2.13 (1.38, 3.30) | 2.03 (1.47, 2.80) | 3.6 · 10⁻³⁰ |
| | | rs3829241 A | 1.60 (1.35, 1.89) | 1.54 (1.12, 2.11) | 1.38 (1.07, 1.77) | 6.2 · 10⁻¹⁶ |
| | ASIP | AH^{a} | 1.45 (1.08, 1.95) | 1.62 (1.08, 2.43) | 1.75 (1.15, 2.66) | 1.7 · 10⁻⁵ |
| | TYRP1 | rs1408799 T | 1.29 (1.09, 1.53) | 1.10 (0.85, 1.42) | 1.10 (0.86, 1.42) | 8.3 · 10⁻⁵ |

^{a}AH is the haplotype G rs1015362 T rs4911414.

TABLE 19
Assosciation of genetic variants with skin sensitivity to sun and freckling in 5,130
Icelandic discovery individuals, 2,116 Icelandic replication individuals and 1,214 Dutch
replication individuals.

| | | | Iceland | | | |
|---|---|---|---|---|---|---|
| | | | Discovery | Replication | Netherlands | |
| | Locus | Variant | OR (95% c.i.) | OR (95% c.i.) | OR (95% c.i.) | P |
| Skin sensitivity to sun | SLC24A4 | rs12896399 T | 1.18 (1.08, 1.30) | 1.02 (0.88, 1.18) | 0.99 (0.84, 1.16) | 0.00012 |
| | KITLG | rs12821256 C | 1.01 (0.90, 1.12) | 1.28 (1.08, 1.52) | 0.84 (0.65, 1.07) | 0.63 |
| | 6P25.3 | rs1540771 A | 1.15 (1.05, 1.26) | 1.10 (0.95, 1.27) | 1.12 (0.95, 1.32) | 6.5 · 10⁻⁶ |
| | TYR | rs1126809 A | 1.32 (1.19, 1.45) | 1.56 (1.34, 1.82) | 1.10 (0.92, 1.32) | 7.1 · 10⁻¹³ |
| | | rs1042602 C | 0.97 (0.88, 1.07) | 1.07 (0.91, 1.25) | 0.86 (0.73, 1.02) | 0.11 |
| | OCA2 | rs1667394 A | 1.23 (1.01, 1.51) | 1.37 (0.98, 1.92) | 1.33 (0.99, 1.80) | 0.00069 |
| | | rs7495174 A | 1.43 (1.04, 1.96) | 0.79 (0.47, 1.31) | 1.67 (1.05, 2.67) | 0.00027 |
| | MC1R | rs1805008 T | 2.34 (2.04, 2.68) | 2.44 (1.98, 3.01) | 1.74 (1.30, 2.33) | 2.3 · 10⁻⁶⁹ |
| | | rs1805007 T | 3.04 (2.59, 3.56) | 3.00 (2.40, 3.73) | 2.12 (1.52, 2.97) | 4.8 · 10⁻⁸⁸ |
| | TPCN2 | rs35264875 T | 1.12 (0.96, 1.31) | 1.17 (0.88, 1.54) | 0.95 (0.76, 1.19) | 0.10 |
| | | rs3829241 A | 1.10 (0.99, 1.22) | 0.92 (0.75, 1.12) | 1.02 (0.86, 1.21) | 0.018 |
| | ASIP | AH^{a} | 1.76 (1.49, 2.08) | 1.82 (1.43, 2.32) | 1.75 (1.32, 2.32) | 2.6 · 10⁻²⁴ |
| | TYRP1 | rs1408799 T | 1.12 (1.01, 1.24) | 1.19 (1.01, 1.41) | 1.01 (0.84, 1.20) | 0.010 |
| Freckles | SLC24A4 | rs12896399 T | 0.97 (0.88, 1.06) | 1.05 (0.91, 1.20) | 1.04 (0.88, 1.22) | 0.44 |
| | KITLG | rs12821256 C | 0.93 (0.83, 1.03) | 1.10 (0.93, 1.30) | 0.96 (0.74, 1.23) | 0.058 |
| | 6P25.3 | rs1540771 A | 1.41 (1.29, 1.54) | 1.27 (1.11, 1.45) | 1.26 (1.06, 1.49) | 6.1 · 10⁻²⁸ |
| | TYR | rs1126809 A | 1.06 (0.97, 1.17) | 1.15 (1.00, 1.33) | 1.09 (0.91, 1.31) | 0.059 |
| | | rs1042602 C | 1.30 (1.18, 1.43) | 1.34 (1.15, 1.55) | 1.23 (1.04, 1.46) | 3.0 · 10⁻¹⁵ |
| | OCA2 | rs1667394 A | 0.98 (0.81, 1.19) | 1.15 (0.84, 1.57) | 1.38 (1.02, 1.87) | 0.36 |
| | | rs7495174 A | 1.08 (0.81, 1.44) | 0.79 (0.49, 1.28) | 1.02 (0.64, 1.62) | 0.56 |
| | MC1R | rs1805008 T | 2.64 (2.30, 3.03) | 2.84 (2.29, 3.51) | 2.29 (1.71, 3.07) | 2.0 · 10⁻⁹⁰ |
| | | rs1805007 T | 4.09 (3.46, 4.83) | 3.07 (2.44, 3.85) | 4.31 (3.05, 6.08) | 3.5 · 10⁻¹³³ |
| | TPCN2 | rs35264875 T | 0.86 (0.75, 1.00) | 1.07 (0.81, 1.41) | 1.30 (1.04, 1.63) | 0.059 |
| | | rs3829241 A | 0.95 (0.86, 1.05) | 0.89 (0.73, 1.08) | 0.92 (0.77, 1.10) | 0.095 |
| | ASIP | AH^{a} | 1.96 (1.65, 2.32) | 2.13 (1.66, 2.72) | 1.56 (1.17, 2.07) | 7.5 · 10⁻²⁹ |
| | TYRP1 | rs1408799 T | 1.02 (0.92, 1.13) | 1.01 (0.86, 1.18) | 0.93 (0.77, 1.11) | 0.74 |
| Skin sensitive to sun and Freckles | SLC24A4 | rs12896399 T | 1.10 (0.97, 1.24) | 1.04 (0.87, 1.25) | 1.08 (0.87, 1.34) | 0.033 |
| | KITLG | rs12821256 C | 0.94 (0.82, 1.09) | 1.31 (1.06, 1.62) | 0.85 (0.61, 1.18) | 0.2 |
| | 6P25.3 | rs1540771 A | 1.48 (1.32, 1.66) | 1.30 (1.09, 1.55) | 1.37 (1.10, 1.70) | 1.3 · 10⁻²¹ |
| | TYR | rs1126809 A | 1.30 (1.14, 1.48) | 1.58 (1.30, 1.91) | 1.13 (0.89, 1.43) | 9.7 · 10⁻⁸ |
| | | rs1042602 C | 1.17 (1.03, 1.33) | 1.31 (1.08, 1.60) | 1.02 (0.82, 1.28) | 0.0017 |
| | OCA2 | rs1667394 A | 1.19 (0.92, 1.55) | 1.41 (0.94, 2.12) | 1.60 (1.06, 2.42) | 0.0082 |
| | | rs7495174 A | 1.44 (0.96, 2.18) | 0.75 (0.40, 1.38) | 1.58 (0.81, 3.10) | 0.0053 |
| | MC1R | rs1805008 T | 4.52 (3.77, 5.42) | 4.44 (3.37, 5.84) | 3.15 (2.14, 4.64) | 4.6 · 10⁻¹²⁴ |
| | | rs1805007 T | 7.32 (5.89, 9.09) | 5.61 (4.19, 7.50) | 5.63 (3.73, 8.49) | 6.3 · 10⁻¹⁵⁷ |
| | TPCN2 | rs35264875 T | 0.97 (0.79, 1.18) | 1.16 (0.83, 1.63) | 1.24 (0.92, 1.66) | 0.64 |
| | | rs3829241 A | 1.02 (0.89, 1.17) | 0.85 (0.66, 1.11) | 0.96 (0.76, 1.22) | 0.87 |
| | ASIP | AH^{a} | 2.55 (2.05, 3.17) | 2.90 (2.11, 3.98) | 2.27 (1.58, 3.26) | 7.1 · 10⁻³⁷ |
| | TYRP1 | rs1408799 T | 1.08 (0.95, 1.24) | 1.17 (0.95, 1.44) | 0.95 (0.75, 1.20) | 0.22 |

^{a}AH is the haplotype G rs1015362 T rs4911414.

### Example 5

### ASIP and TYR Pigmentation Variants Associate with Cutaneous Melanoma and Basal Cell Carcinoma

Cutaneous melanoma (CM) is a rare malignant tumor of melanocytes that, due to its aggressive nature, causes the majority of skin cancer related deaths¹. Basal cell carcinoma (BCC) is the most common skin neoplasm² but is unlikely to metastasize. UV, through a complex mechanism, exposure is a known risk factor for both CM and BCC³⁻⁵. Pale skin with poor tanning response, red or blonde hair, blue or green eyes and freckles are known risk factors and are thought to act predominantly through reduced protection from UV irradiation⁶.

Several missense mutations in the MC1R (encoding melanocortin 1 receptor) gene have been previously associated with skin cancers in addition to their effect on pigmentation⁷⁻¹². We recently identified several genetic determinants of hair, eye and skin pigmentation in Europeans^{13,14}. In addition to the known MC1R variants, we directly assessed the association of eleven distinct sequence variants at eight loci (Table 20) with risk of CM and BCC in an Icelandic sample of 810 CM cases and 36,723 non CM controls, an Icelandic sample of 1,649 BCC cases and 33,824 non BCC controls, a Swedish sample of 1,033 CM cases and 2,650 controls, and a Spanish sample of 278 CM cases and 1,297 controls. The association results for the eight loci tested are listed in Table 21 and Table 22). Variants at three of the eight loci, ASIP (encoding agouti signaling protein), TYR (encoding tyrosinase), and TYRP1 (encoding tyrosinase related protein 1), showed significant association to CM after correcting for the number of tests performed (P<0.05/22=0.0023). The variants at ASIP and TYR also associated to BCC in Iceland (Table 21 and Table 22). The variants at the three loci were then further tested in an Eastern European sample from Hungary, Romania and Slovakia of 514 BCC cases and 522 controls¹⁵ and the association with BCC was replicated for ASIP and TYR.

A two-SNP haplotype (ASIP haplotype, AH), rs1015362 G and rs4911414 T, at the ASIP locus was the variant most strongly associated with both CM (combined for all three CM samples OR=1.45, P=1.2·10⁻⁹) and BCC (combined for the two samples OR=1.35, P=1.2·10⁻⁶). The ASIP gene product, agouti signaling protein, antagonizes the interaction between the melanocortin 1 receptor and α-melanocyte stimulating hormone, bringing about a pheomelanin response^{16,17}. This would suggest that the causative variant underlying the ASIP haplotype is a gain-of-function mutation. Because of its function, ASIP has long been considered a candidate for a gene affecting skin cancers. Previous studies showed association of a polymorphism in the 3′ untranslated region of ASIP (rs6058017, 8818A>G) with pigmentation characteristics¹⁸⁻²⁰. This association is much smaller in magnitude than that with AH¹⁴ and attempts to associate this variant with melanoma have failed^{19,20}. In Europeans, AH has frequency under 10% and occurs on the background of the major allele of rs6058017, which has frequency around 90%, and the correlation between the two is weak (D′=1, r²=0.008). Importantly, we did not observe an association of rs6058017 with either CM or BCC (Table 21).

The R402Q (rs1126809) mutation in TYR showed the second most significant association to CM (combined for all three samples OR=1.21, P=2.8·10⁻⁷) and BCC (combined for the two samples OR=1.14, P=0.00061). R402Q is a common mutation in the tyrosinase gene associated with a mild, temperature-sensitive variant form of albinism (OCA1-TS)²¹.

Allele C of rs1408799 at the TYRP1 locus, associated significantly with CM (combined for all three samples OR=1.15, P=0.00043), but not with BCC (combined for both samples OR=1.05, P=0.20).

No pigmentation trait-associated variant in the SLC24A4, KITLG, 6p25.3, OCA2, or TPCN2 loci showed even nominally significant association with risk of CM or BCC (Table 22). Among these variants is a SNP on 6p25.3 that associates with freckling and skin sensitivity to sun and SNPs in SLC24A4 and OCA2 that show weak association to skin sensitivity to sun of similar magnitude as TYRP^{13,14}. Thus, not all genetic variants underlying these pigmentation traits confer detectable risk of skin cancer.

The Icelandic and Swedish samples included both invasive and in situ melanoma cases (Tables 21 and 22). The results for the Swedish invasive cases are similar to the in situ cases for the variants at ASIP, TYR and TYRP1, whereas in Iceland the association appears to be born mostly by the invasive cases (Table 21). Taking into account that the Icelandic cancer registry has been recording malignant melanoma cases since 1955, but only started recording in situ melanoma cases in 1980, there is a substantially higher percentage of in situ cases in the Icelandic sample than the Swedish one. These differences in the relative frequency of in situ melanoma and in the strength of genetic association may be due to the different sample ascertainment, with the Icelandic sample being based on the national cancer registry and the Swedish on hospital ascertainment, which may then be less susceptible to over- or misdiagnoses.

Available pigmentation characteristics do not completely account for the reported association of variants in MC1R with CM¹⁰ and BCC¹². In the Icelandic sample, the same appears to be true for the association of the ASIP, TYR and TYRP1 variants, where risk estimates are robust to adjustment for the risk of skin cancers conferred by hair, eye and skin pigmentation (Table 23). This may be because the self-reported pigmentation trait assessment does not adequately reflect those aspects of pigmentation status that relate best to skin cancer risk. It may also indicate that ASIP, TYR and TYRP1 have risk-associated functions that are not directly related to easily observed pigmentation traits, as has been previously suggested for MC1R²⁴. Both the ASIP and TYR variants show stronger association with CM in individuals whose skin is not sensitive to sun. This is a trend similar to that previously reported for MC1R variants when stratifying on skin color^{10,12}. Pigmentation information was not collected for both cases and controls for any of the non-Icelandic samples.

For all variants associating with CM a trend towards earlier age at diagnosis was observed (Table 24). However, this trend was only nominally significant for AH at ASIP (diagnosis was 2.00 years earlier per copy, P=0.029).

Most variants that affect pigmentation in Europeans have been subject to strong selection. The population frequencies in the north and south of Europe differ and they also differ between Europeans and populations of other ethnicities¹³. Associating these SNPs to traits like skin cancers which are also known to have geographic differences in incidence is therefore particularly sensitive to artifacts due to population stratification. However, the ancestry informative variants that we studied, in OCA2, KITLG and TPCN2, did not associate with either CM or BCC, convincing us that the association to ASIP and TYR is not due to bias rooted in stratification, a possibility that is also made unlikely by replication in several populations and in samples ascertained in more than one way. However, the more modest association with TYRP1, calls for further validation in other populations.

Following the discovery of mutations in MC1R affecting pigmentation characteristics, these same mutations were also associated with the risk of skin cancers even after taking the available assessment of pigmentation into account. This path has now been retraced for variants at the ASIP and TYR loci, highlighting the importance of studying pigmentation for identification of sequence variants predisposing to skin cancers. This is particularly true for ASIP, encoding a protein that interacts with MC1R, where sequence variants near the gene itself have failed to show association to skin cancers, but the new variants identified through their association to pigmentation characteristics show strong evidence for association with CM and BCC.

Methods

Patients and Control Selection:

Iceland: Approval for the study was granted by the Icelandic National Bioethics Committee and the Icelandic Data Protection Authority. Records of cutaneous invasive malignant melanoma diagnoses, all histologically confirmed, from the years 1955-2007 were obtained from the Icelandic Cancer Registry (ICR). Invasive cutaneous malignant melanoma (CMM) was identified through ICD10 code C43. The ICR records also included diagnoses of melanoma in situ from 1980-2007, identified by ICD10 code D03. Metastatic melanoma (where the primary lesion had not been identified) was identified by a SNOMED morphology code indicating melanoma with a/6 suffix, regardless of the ICD10 code. Ocular melanoma (OM) and melanomas arising at mucosal sites were not included. Diagnoses of BCC were recorded by the ICR from 1981-2007 and were identified by ICD10 code C44 with a SNOMED morphology code indicating basal cell carcinoma.

All patients identified through the ICR were invited to a study recruitment center where they signed an informed consent form and provided a blood sample. Melanoma patients (n=635) and controls (n=6,980) answered a questionnaire with the aid of a study nurse. The questionnaire included questions about natural hair and eye color, freckling amount (none, few, moderate, many), and tanning responses using the Fitzpatrick scale. Questions were also included asking the numbers of mild and severe sunburns suffered as a child, teenager and adult.

The Icelandic controls consisted of individuals selected from other ongoing association studies at deCODE. Individuals with at diagnosis of melanoma or BCC as well as their first and second degree relatives, were excluded from the respective control groups. For the analysis of variants in MC1R, fewer controls were available because genotypes for these variants could not be derived from SNPs represented on the Illumina chips. These controls were derived from participants in family studies on breast cancer and melanoma. Patients with melanoma or BCC and their first and second degree relatives, as identified from the Icelandic Genealogical Database were excluded from this control set. There were no significant differences between genders in the frequencies of the SNPs studied and no association with age. All subjects were of European ethnicity.

Sweden: The Swedish sample was composed of 1069 consecutive patients attending care for cutaneous malignant melanoma (CMM) at the Karolinska University Hospital in Solna during 1993 to 2007. The clinical characteristics of the subjects were obtained from medical records. All patients had at least one pathologically confirmed CMM, including in-situ melanomas. 831 of the patients had one single primary melanoma whereas 163 cases had at least two independent primary CMMs and were therefore considered to be multiple primary melanoma patients. Single or multiple primary melanoma status was not recorded for 75 patients. None of the patients had a known family history of CMM. The median age at diagnosis was 60 years (range 17-91).

The controls were blood donors recruited on a voluntary basis (N=2000), newborns (N=202, where placental tissue was used for DNA preparation) and 448 cancer-free individuals recruited from the Karolinska University Hospital, Stockholm. All subjects originated from the Stockholm region except for the 202 newborns, who originated from Northern Sweden and 202 blood donors originating from Southern Sweden. The study was conducted in accordance with the Declaration of Helsinki. Ethical approval for the study from the local ethics committee and written informed consent from all study participants were obtained.

Spain: 180 of the Spanish study patients were recruited from the Department of Dermatology, Valencia Institute of Oncology. This is a referral centre for skin cancer for the provinces of Valencia, Alicante, and Casteón, a catchment population of approximately 5 million people. The samples were collected from patients visiting the centre from May 2000, including newly diagnosed patients and those attending follow-up examinations. All diagnoses were confirmed by histopathology. Median age at diagnosis was 54 years (range 15-85). All subjects were of European ethnicity.

93 of the Spanish study patients were recruited from the Oncology Department of Zaragoza Hospital between September 2006 and February 2008. Patients with histologically-proven invasive cutaneous melanoma or metastatic melanoma were eligible to participate in the study. The median time interval from melanoma diagnosis to collection of blood samples was 11 months (mean 16 months, range 1-49 months). The median age at diagnosis was 58 years (range 23-90). The 1540 Spanish controls had attended the University Hospital in Zaragoza for diseases other than cancer. Controls were questioned to rule out prior cancers before drawing the blood sample. All patients and controls were of European ethnicity. Ethical approval for the Spanish part of the study was given by the local ethics committees and written informed consent from all study participants were obtained.

Eastern Europe: Details of this case: control set have been published previously¹⁵. Briefly, BCC cases were recruited from all general hospitals in three study areas in Hungary, two in Romania and one in Slovakia. Patients were identified on the basis of histopatholgical examinations by pathologists. The median age at diagnosis was 67 years (range 30-85). Controls were recruited from the same hospitals. Individuals with malignant disease, cardiovascular disease and diabetes were excluded. Local ethical boards approved of the study.

Genotyping

Approximately 800 Icelandic BCC patients, all Icelandic CM patients and controls were genotyped on Illumina HumanHap300 or HumanCNV370-duo chips as described previously²⁷. Other SNP genotyping was carried out using Nanogen Centaurus assay²⁸. Primer sequences are available on request. Centaurus SNP assays were validated by genotyping the HapMap CEU samples and comparing genotypes to published data. Assays were rejected if they showed ≧1.5% mismatches with the HapMap data. Approximately 10% of the Icelandic case samples that were genotyped on the Illumina platform were also genotyped using the Centaurus assays and the observed mismatch rate was lower than 0.5%. Supplemenatary Table 6 contains overview of quality control statistics for the genotyping of the SNPs reported in key tables.

The single coding exon of MC1R was sequenced in 703 melanoma cases and 691 population-based controls using the ABI PRISM Dye Terminator system and Applied Biosytems 3730 Sequencers. SNP calling from primary sequence data was carried out using deCODE Genetics' Sequence Miner software. Sixteen different MC1R variants were identified: 13 missense variants, 2 synonymous coding variants and one 5′ untranslated sequence variant. Centaurus assays were generated for the following common variants: V60L, D84E, V92M, R151C, I155T, R160W, D294H and T314T, and were used for genotyping in all other samples.

Statistical Analysis

We calculated the OR for each SNP allele or haplotype assuming the multiplicative model; i.e. assuming that the relative risk of the two alleles that a person carries multiplies. Allelic frequencies and OR are presented for the markers. The associated P values were calculated with the standard likelihood ratio X² statistic as implemented in the NEMO software package²⁹. Confidence intervals were calculated assuming that the estimate of OR has a log-normal distribution. For SNPs that were in strong LD, whenever the genotype of one SNP was missing for an individual, the genotype of the correlated SNPs were used to impute genotypes through a likelihood approach as previously described²⁹. This ensured that results presented for different SNPs were based on the same number of individuals, allowing meaningful comparisons of OR and P-values.

Some of the Icelandic patients and controls are related to each other, both within and between groups, causing the X² statistic to have a mean>1. We estimated the inflation factor by simulating genotypes through the Icelandic genealogy, as described previously³⁰, and corrected the X² statistics for Icelandic OR's accordingly. The estimated inflation factor was 1.03 for CM in Iceland and 1.11 for BCC in Iceland.

Joint analyses of multiple case-control replication groups were carried out using a Mantel-Haenszel model in which the groups were allowed to have different population frequencies for alleles or genotypes but were assumed to have common relative risks. The tests of heterogeneity were performed by assuming that the allele frequencies were the same in all groups under the null hypothesis, but each group had a different allele frequency under the alternative hypothesis. Joint analyses of multiple groups of cases were performed using an extended Mantel-Haenszel model that corresponds to a polytomous logistic regression using the group indicator as a covariate.

The same Mantel-Haenszel model was used to combine the results from Eastern Europe which came from 5 strata: Hungarians living in Hungary, Hungarians living in Romania, Hungarians living in Slovakia, Romanians living in Romania, and Slovaks living in Slovakia.

We calculated genotype specific ORs, by estimating the genotype frequencies in the population assuming Hardy-Weinberg equilibrium. No significant deviations from multiplicity were observed for the SNPs showing association to skin cancer.

All P values are reported as two-sided.

### REFERENCES

1. Jemal, A. et al. Cancer statistics, 2007. CA Cancer J Clin 57, 43-66 (2007).

2. Miller, D. L. & Weinstock, M. A. Nonmelanoma skin cancer in the United States: incidence. J Am Acad Dermatol 30, 774-8 (1994).

3. Markovic, S. N. et al. Malignant melanoma in the 21st century, part 1: epidemiology, risk factors, screening, prevention, and diagnosis. Mayo Clin Proc 82, 364-80 (2007).

4. Oliveria, S. A., Saraiya, M., Geller, A. C., Heneghan, M. K. & Jorgensen, C. Sun exposure and risk of melanoma. Arch Dis Child 91, 131-8 (2006).

5. Madan, V., Hoban, P., Strange, R. C., Fryer, A. A. & Lear, J. T. Genetics and risk factors for basal cell carcinoma. Br J Dermatol 154 Suppl 1, 5-7 (2006).

6. Han, J., Colditz, G. A. & Hunter, D. J. Risk factors for skin cancers: a nested case-control study within the Nurses' Health Study. Int J Epidemiol 35, 1514-21 (2006).

7. Valverde, P. et al. The Asp84Glu variant of the melanocortin 1 receptor (MC1R) is associated with melanoma. Hum Mol Genet 5, 1663-6 (1996).

8. Bastiaens, M. T. et al. Melanocortin-1 receptor gene variants determine the risk of nonmelanoma skin cancer independently of fair skin and red hair. Am J Hum Genet 68, 884-94 (2001).

9. Han, J., Kraft, P., Colditz, G. A., Wong, J. & Hunter, D. J. Melanocortin 1 receptor variants and skin cancer risk. Intl Cancer 119, 1976-84 (2006).

10. Palmer, J. S. et al. Melanocortin-1 receptor polymorphisms and risk of melanoma: is the association explained solely by pigmentation phenotype? Am J Hum Genet 66, 176-86 (2000).

11. Kennedy, C. et al. Melanocortin 1 receptor (MC1R) gene variants are associated with an increased risk for cutaneous melanoma which is largely independent of skin type and hair color. J Invest Dermatol 117, 294-300. (2001).

12. Box, N. F. et al. Melanocortin-1 receptor genotype is a risk factor for basal and squamous cell carcinoma. J Invest Dermatol 116, 224-9 (2001).

13. Sulem, P. et al. Genetic determinants of hair, eye and skin pigmentation in Europeans. Nat Genet 39, 1443-52 (2007).

14. Sulem, P.e.a. Novel genetic determinants of pigmentation in Europeans. Submitted to Nat Genet (2008).

15. Scherer, D. et al. MC1R variants associated susceptibility to basal cell carcinoma of skin interaction with host factors and XRCC3 polymorphism. Int J Cancer 122, 1787-93 (2008).

16. Voisey, J. & van Daal, A. Agouti: from mouse to man, from skin to fat. Pigment Cell Res 15, 10-8 (2002).

17. Suzuki, I. et al. Agouti signaling protein inhibits melanogenesis and the response of human melanocytes to alpha-melanotropin. J Invest Dermatol 108, 838-42 (1997).

18. Kanetsky, P. A. et al. A polymorphism in the agouti signaling protein gene is associated with human pigmentation. Am J Hum Genet 70, 770-5 (2002).

19. Landi, M. T. et al. MC1R, ASIP, and DNA repair in sporadic and familial melanoma in a Mediterranean population. J Natl Cancer Inst 97, 998-1007 (2005).

20. Meziani, R. et al. Association study of the g.8818A>G polymorphism of the human agouti gene with melanoma risk and pigmentary characteristics in a French population. J Dermatol Sci 40, 133-6 (2005).

21. King, R. A. et al. Temperature-sensitive tyrosinase associated with peripheral pigmentation in oculocutaneous albinism. J Clin Invest 87, 1046-53 (1991).

22. Frudakis, T. et al. Sequences associated with human iris pigmentation. Genetics 165, 2071-83 (2003).

23. Wong, T. H. & Rees, J. L. The relation between melanocortin 1 receptor (MC1R) variation and the generation of phenotypic diversity in the cutaneous response to ultraviolet radiation. Peptides 26, 1965-71 (2005).

24. Rees, J. Plenty new under the sun. J Invest Dermatol 126, 1691-2 (2006).

25. Sturm, R. A. & Frudakis, T. N. Eye colour: portals into pigmentation genes and ancestry. Trends Genet 20, 327-32 (2004).

26. Duffy, D. L. et al. A three-single-nucleotide polymorphism haplotype in intron 1 of OCA2 explains most human eye-color variation. Am J Hum Genet 80, 241-52 (2007).

27. Stacey, S. N. et al. Common variants on chromosomes 2q35 and 16q12 confer susceptibility to estrogen receptor-positive breast cancer. Nat Genet 39, 865-9 (2007).

28. Kutyavin, I. V. et al. A novel endonuclease IV post-PCR genotyping system. Nucleic Acids Res 34, e128 (2006).

29. Gretarsdottir, S. et al. The gene encoding phosphodiesterase 4D confers risk of ischemic stroke. Nat Genet 35, 131-8. Epub 2003 Sep. 21. (2003).

30. Grant, S. F. et al. Variant of transcription factor 7-like 2 (TCF7L2) gene confers risk of type 2 diabetes. Nat Genet 38, 320-3 (2006).

TABLE 20
The 11 variants known to associated with pigmentation
being tested for association to skin cancers.

| Gene/Locus | SNP | Primary pigmentation association |
|---|---|---|
| SLC24A4 | rs12896399 T | Blonde vs. brown hair¹³ |
| KITLG | rs12821256 C | Blonde vs. brown hair¹³ |
| 6P25.3 | rs1540771 A | Freckling¹³ |
| TYR | rs1126809 A | Blue vs. green eyes¹³ |
| | rs1042602 C | Freckling¹³ |
| OCA2 | rs1667394 A | Blue vs. brown eyes^{13,22,25,26} |
| | rs7495174 A | Blue vs. brown eyes^{13,22,25,26} |
| TPCN2 | rs35264875 T | Blonde vs. brown hair¹⁴ |
| | rs3829241 A | Blonde vs. brown hair¹⁴ |
| ASIP | AH^{a} | Freckling and burning¹⁴ |
| TYRP1 | rs1408799 T | Blue vs. green or brown eyes^{14,22} |

TABLE 21
Association analysis of pigmentation variants with CM in Icelandic, Swedish and
Spanish samples and BCC in Icelandic and Eastern European samples. Also presented is the
previously studied ASIP polymorphism 8818A > G.

| Locus | | Number | | Frequency | | | |
|---|---|---|---|---|---|---|---|
| Variant | Sample | Cases | Control | Case | Control | OR (95% CI) | P |
| ASIP | Iceland invasive | 565 | 36,147 | 0.118 | 0.081 | 1.52 (1.26, 1.85) | 2.1 · 10⁻⁵ |
| AH | CM | | | | | | |
| | Iceland in situ | 245 | 36,147 | 0.078 | 0.081 | 0.97 (0.68, 1.37) | 0.85 |
| | CM | | | | | | |
| | Iceland CM | 810 | 36,147 | 0.106 | 0.081 | 1.35 (1.14, 1.60) | 0.00045 |
| | Sweden | 753 | 2,650 | 0.101 | 0.067 | 1.56 (1.27, 1.92) | 2.6 · 10⁻⁵ |
| | invasive CM | | | | | | |
| | Sweden in situ | 162 | 2,650 | 0.109 | 0.067 | 1.71 (1.15, 2.52) | 0.0073 |
| | CM | | | | | | |
| | Sweden CM | 1,033 | 2,650 | 0.099 | 0.067 | 1.53 (1.27, 1.84) | 8.6 · 10⁻⁶ |
| | Spain invasive | 268 | 1,297 | 0.061 | 0.035 | 1.80 (1.16, 2.80) | 0.0089 |
| | CM | | | | | | |
| | Iceland BCC | 1,636 | 33,320 | 0.104 | 0.081 | 1.32 (1.17, 1.50) | 1.4 · 10⁻⁵ |
| | Eastern Europe | 514 | 522 | 0.062 | 0.037 | 1.74 (1.12, 2.72) | 0.014 |
| | BCC | | | | | | |
| | All CM | | | | | 1.45 (1.29, 1.64) | 1.2 · 10⁻⁹ |
| | All BCC | | | | | 1.35 (1.20, 1.53) | 1.2 · 10⁻⁶ |
| rs6058017 A | Iceland invasive | 565 | 36,147 | 0.911 | 0.915 | 0.95 (0.78, 1.15) | 0.59 |
| (8818A > G) | CM | | | | | | |
| | Iceland in situ | 245 | 36,147 | 0.933 | 0.915 | 1.30 (0.88, 1.90) | 0.18 |
| | CM | | | | | | |
| | Iceland CM | 810 | 36,147 | 0.917 | 0.916 | 1.02 (0.86, 1.21) | 0.86 |
| | Sweden | 753 | 2,650 | 0.899 | 0.887 | 1.13 (0.94, 1.36) | 0.19 |
| | invasive CM | | | | | | |
| | Sweden in situ | 162 | 2,650 | 0.896 | 0.887 | 1.10 (0.76, 1.59) | 0.62 |
| | CM | | | | | | |
| | Sweden CM | 1,033 | 2,650 | 0.898 | 0.887 | 1.12 (0.95, 1.31) | 0.18 |
| | Spain invasive | 268 | 1,297 | 0.881 | 0.857 | 1.24 (0.93, 1.63) | 0.14 |
| | CM | | | | | | |
| | Iceland BCC | 1,636 | 33,320 | 0.922 | 0.917 | 1.07 (0.90, 1.29) | 0.44 |
| | Eastern Europe | 514 | 522 | 0.855 | 0.854 | 1.01 (0.79, 1.29) | 0.95 |
| | BCC | | | | | | |
| | All CM | | | | | 1.09 (0.98, 1.22) | 0.11 |
| | All BCC | | | | | 1.05 (0.91, 1.22) | 0.51 |
| TYR | Iceland invasive | 565 | 36,723 | 0.335 | 0.301 | 1.17 (1.03, 1.33) | 0.016 |
| rs1126809 A | CM | | | | | | |
| (R402Q) | Iceland in situ | 245 | 36,723 | 0.312 | 0.302 | 1.05 (0.86, 1.27) | 0.64 |
| | CM | | | | | | |
| | Iceland CM | 810 | 36,723 | 0.328 | 0.301 | 1.13 (1.02, 1.26) | 0.023 |
| | Sweden | 753 | 2,648 | 0.309 | 0.255 | 1.31 (1.15, 1.49) | 3.3 · 10⁻⁵ |
| | invasive CM | | | | | | |
| | Sweden in situ | 162 | 2,648 | 0.308 | 0.255 | 1.30 (1.01, 1.67) | 0.038 |
| | CM | | | | | | |
| | Sweden CM | 1,033 | 2,648 | 0.311 | 0.255 | 1.32 (1.18, 1.48) | 1.4 · 10⁻⁶ |
| | Spain invasive | 268 | 1,228 | 0.289 | 0.260 | 1.16 (0.94 1.43), | 0.16 |
| | CM | | | | | | |
| | Iceland BCC | 1,649 | 33,824 | 0.326 | 0.300 | 1.13 (1.04, 1.22) | 0.0035 |
| | Eastern Europe | 514 | 522 | 0.258 | 0.221 | 1.23 (1.00, 1.51) | 0.050 |
| | BCC | | | | | | |
| | All CM | | | | | 1.21 (1.13, 1.30) | 2.8 · 10⁻⁷ |
| | All BCC | | | | | 1.14 (1.06, 1.23) | 0.00061 |
| TYRP1 | Iceland invasive | 565 | 36,125 | 0.788 | 0.748 | 1.25 (1.08, 1.44) | 0.0021 |
| rs1408799 C | CM | | | | | | |
| | Iceland in situ | 245 | 36,125 | 0.763 | 0.748 | 1.09 (0.88, 1.34) | 0.44 |
| | CM | | | | | | |
| | Iceland CM | 810 | 36,125 | 0.780 | 0.748 | 1.20 (1.06, 1.35) | 0.0029 |
| | Sweden | 753 | 2,640 | 0.744 | 0.734 | 1.05 (0.93, 1.20) | 0.42 |
| | invasive CM | | | | | | |
| | Sweden in situ | 162 | 2,640 | 0.765 | 0.734 | 1.18 (0.91, 1.54) | 0.20 |
| | CM | | | | | | |
| | Sweden CM | 1,032 | 2,640 | 0.750 | 0.734 | 1.09 (0.97, 1.22) | 0.15 |
| | Spain invasive | 268 | 1,278 | 0.681 | 0.643 | 1.18 (0.97, 1.44) | 0.096 |
| | CM | | | | | | |
| | Iceland BCC | 1,634 | 33,300 | 0.754 | 0.748 | 1.03 (0.95, 1.13) | 0.43 |
| | CM | | | | | | |
| | Eastern Europe | 507 | 515 | 0.689 | 0.659 | 1.14 (0.95, 1.38) | 0.17 |
| | BCC | | | | | | |
| | All CM | | | | | 1.15 (1.06, 1.24) | 0.00043 |
| | All BCC | | | | | 1.05 (0.97, 1.14) | 0.20 |

TABLE 22
Association of additional pigmentation variants to CM and BCC.

| Locus | | Number | | Frequency | | | |
|---|---|---|---|---|---|---|---|
| Variant | Sample | Cases | Control | Case | Control | OR (95% CI) | P |
| KITLG | | | | | | | |
| rs12821256 C | Iceland invasive | 565 | 33,497 | 0.215 | 0.201 | 1.09 (0.94, 1.26) | 0.25 |
| | CM | | | | | | |
| | Iceland in situ CM | 245 | 33,497 | 0.229 | 0.201 | 1.18 (0.95, 1.47) | 0.14 |
| | Iceland CM | 810 | 33,497 | 0.219 | 0.201 | 1.12 (0.99, 1.26) | 0.078 |
| | Sweden invasive | 753 | 2,639 | 0.164 | 0.177 | 0.92 (0.79, 1.07) | 0.26 |
| | CM | | | | | | |
| | Sweden in situ CM | 162 | 2,639 | 0.183 | 0.177 | 1.05 (0.78, 1.40) | 0.76 |
| | Sweden CM | 1,033 | 2,639 | 0.170 | 0.177 | 0.96 (0.84, 1.09) | 0.51 |
| | Spain invasive CM | 268 | 1,268 | 0.032 | 0.038 | 0.83 (0.49, 1.38) | 0.46 |
| | Iceland BCC | 1,635 | 30,949 | 0.201 | 0.201 | 1.00 (0.91, 1.09) | 0.96 |
| | All CM | | | | | 1.03 (0.95, 1.13) | 0.47 |
| OCA2 | | | | | | | |
| rs7495174 A | Iceland invasive | 565 | 33,508 | 0.975 | 0.973 | 1.08 (0.74, 1.58) | 0.69 |
| | CM | | | | | | |
| | Iceland in situ CM | 245 | 33,508 | 0.969 | 0.973 | 0.87 (0.51, 1.48) | 0.60 |
| | Iceland CM | 810 | 33,508 | 0.974 | 0.973 | 1.01 (0.71, 1.41) | 0.97 |
| | Sweden invasive | 753 | 2,647 | 0.974 | 0.972 | 1.08 (0.77, 1.52) | 0.67 |
| | CM | | | | | | |
| | Sweden in situ CM | 162 | 2,647 | 0.970 | 0.972 | 0.93 (0.50, 1.73) | 0.82 |
| | Sweden CM | 1,033 | 2,647 | 0.972 | 0.972 | 1.00 (0.14, 7.10) | 1.00 |
| | Spain invasive CM | 268 | 1,286 | 0.832 | 0.836 | 0.97 (0.76, 1.24) | 0.80 |
| | Iceland BCC | 1,636 | 30,964 | 0.976 | 0.973 | 1.09 (0.86, 1.39) | 0.45 |
| | All CM | | | | | 0.98 (0.80, 1.20) | 0.85 |
| rs1667394 A | Iceland invasive | 565 | 33,508 | 0.949 | 0.939 | 1.20 (0.92, 1.56) | 0.18 |
| | CM | | | | | | |
| | Iceland in situ CM | 245 | 33,508 | 0.924 | 0.939 | 0.79 (0.56, 1.13) | 0.20 |
| | Iceland CM | 810 | 33,508 | 0.941 | 0.939 | 1.04 (0.84, 1.29) | 0.71 |
| | Sweden invasive | 753 | 2,647 | 0.934 | 0.931 | 1.04 (0.83, 1.31) | 0.71 |
| | CM | | | | | | |
| | Sweden in situ CM | 162 | 2,647 | 0.944 | 0.931 | 1.24 (0.78, 1.99) | 0.37 |
| | Sweden CM | 1,033 | 2,647 | 0.932 | 0.931 | 1.02 (0.83, 1.24) | 0.87 |
| | Spain invasive CM | 268 | 1,286 | 0.621 | 0.596 | 1.11 (0.92, 1.35) | 0.28 |
| | Iceland BCC | 1,636 | 30,964 | 0.936 | 0.939 | 0.94 (0.81, 1.10) | 0.47 |
| | All CM | | | | | 1.06 (0.94, 1.19) | 0.34 |
| 6p25.3 | | | | | | | |
| rs1540771 A | Iceland invasive | 563 | 33,403 | 0.467 | 0.463 | 1.02 (0.90, 1.15) | 0.77 |
| | CM | | | | | | |
| | Iceland in situ CM | 244 | 33,403 | 0.443 | 0.463 | 0.92 (0.77, 1.11) | 0.38 |
| | Iceland CM | 807 | 33,403 | 0.460 | 0.463 | 0.99 (0.89, 1.09) | 0.81 |
| | Sweden invasive | 723 | 2,517 | 0.450 | 0.441 | 1.04 (0.92, 1.17) | 0.55 |
| | CM | | | | | | |
| | Sweden in situ CM | 154 | 2,517 | 0.471 | 0.441 | 1.13 (0.90, 1.42) | 0.30 |
| | Sweden CM | 994 | 2,517 | 0.445 | 0.441 | 1.02 (0.92, 1.13) | 0.73 |
| | Spain invasive CM | 268 | 1,161 | 0.511 | 0.533 | 0.92 (0.76, 1.11) | 0.37 |
| | Iceland BCC | 1,621 | 30,874 | 0.452 | 0.464 | 0.95 (0.88, 1.03) | 0.20 |
| | All CM | | | | | 0.99 (0.93, 1.06) | 0.80 |
| SLC24A4 | | | | | | | |
| rs12896399 T | Iceland invasive | 565 | 33,882 | 0.558 | 0.554 | 1.02 (0.90, 1.15) | 0.79 |
| | CM | | | | | | |
| | Iceland in situ CM | 245 | 33,882 | 0.578 | 0.555 | 1.10 (0.91, 1.32) | 0.32 |
| | Iceland CM | 810 | 33,882 | 0.564 | 0.555 | 1.04 (0.94, 1.15) | 0.44 |
| | Sweden invasive | 724 | 2,581 | 0.564 | 0.531 | 1.14 (1.02, 1.28) | 0.026 |
| | CM | | | | | | |
| | Sweden in situ CM | 161 | 2,581 | 0.528 | 0.531 | 0.99 (0.79, 1.24) | 0.91 |
| | Sweden CM | 998 | 2,581 | 0.558 | 0.531 | 1.11 (1.00, 1.24) | 0.040 |
| | Spain invasive CM | 268 | 1,191 | 0.312 | 0.374 | 0.76 (0.62, 0.93) | 0.0064 |
| | Iceland BCC | 1,635 | 31,307 | 0.570 | 0.553 | 1.07 (1.00, 1.16) | 0.057 |
| | All CM | | | | | 1.03 (0.97, 1.11) | 0.35 |
| TPCN2 | | | | | | | |
| rs3829241 A | Iceland invasive | 564 | 36,092 | 0.457 | 0.434 | 1.10 (0.98, 1.24) | 0.12 |
| | CM | | | | | | |
| | Iceland in situ CM | 245 | 36,092 | 0.467 | 0.434 | 1.14 (0.95, 1.37) | 0.15 |
| | Iceland CM | 809 | 36,092 | 0.460 | 0.434 | 1.11 (1.01, 1.23) | 0.037 |
| | Sweden invasive | 753 | 2,634 | 0.405 | 0.395 | 1.04 (0.93, 1.18) | 0.47 |
| | CM | | | | | | |
| | Sweden in situ CM | 162 | 2,634 | 0.381 | 0.395 | 0.94 (0.75, 1.18) | 0.60 |
| | Sweden CM | 1,033 | 2,634 | 0.404 | 0.395 | 1.04 (0.94, 1.15) | 0.47 |
| | Spain invasive CM | 268 | 1,264 | 0.353 | 0.378 | 0.90 (0.74, 1.09) | 0.27 |
| | Iceland BCC | 1,636 | 33,263 | 0.426 | 0.435 | 0.96 (0.89, 1.04) | 0.33 |
| | All CM | | | | | 1.05 (0.98, 1.13) | 0.14 |
| rs35264875 T | Iceland invasive | 564 | 36,092 | 0.223 | 0.217 | 1.03 (0.89, 1.19) | 0.67 |
| | CM | | | | | | |
| | Iceland in situ CM | 245 | 36,092 | 0.197 | 0.216 | 0.89 (0.71, 1.11) | 0.31 |
| | Iceland CM | 809 | 36,092 | 0.215 | 0.217 | 0.99 (0.88, 1.12) | 0.85 |
| | Sweden invasive | 753 | 2,634 | 0.240 | 0.225 | 1.09 (0.95, 1.25) | 0.24 |
| | CM | | | | | | |
| | Sweden in situ CM | 162 | 2,634 | 0.210 | 0.225 | 0.91 (0.70, 1.19) | 0.51 |
| | Sweden CM | 1,033 | 2,634 | 0.234 | 0.225 | 1.05 (0.93, 1.19) | 0.43 |
| | Spain invasive CM | 268 | 1,264 | 0.141 | 0.128 | 1.12 (0.85, 1.47) | 0.43 |
| | Iceland BCC | 1,636 | 33,263 | 0.220 | 0.217 | 1.02 (0.93, 1.11) | 0.69 |
| | All CM | | | | | 1.03 (0.95, 1.11) | 0.53 |
| TYR | | | | | | | |
| rs1042602 C | Iceland invasive | 565 | 36,723 | 0.734 | 0.701 | 1.17 (1.03, 1.34) | 0.018 |
| (s192Y) | CM | | | | | | |
| | Iceland in situ CM | 245 | 36,723 | 0.696 | 0.701 | 0.98 (0.80, 1.19) | 0.81 |
| | Iceland CM | 810 | 36,723 | 0.722 | 0.701 | 1.11 (0.99, 1.24) | 0.069 |
| | Sweden invasive | 753 | 2,648 | 0.687 | 0.697 | 0.95 (0.84, 1.08) | 0.46 |
| | CM | | | | | | |
| | Sweden in situ CM | 162 | 2,648 | 0.698 | 0.697 | 1.00 (0.78, 1.28) | 0.98 |
| | Sweden CM | 1,033 | 2,648 | 0.695 | 0.697 | 0.99 (0.89, 1.11) | 0.90 |
| | Spain invasive CM | 268 | 1,228 | 0.565 | 0.547 | 1.08 (0.89, 1.30) | 0.44 |
| | Iceland BCC | 1,649 | 33,824 | 0.697 | 0.704 | 0.97 (0.90, 1.05) | 0.47 |
| | All CM | | | | | 1.05 (0.98, 1.13) | 0.17 |

TABLE 23
Association between ASIP, TYR, TYRP1 and MC1R variants and CM
in Iceland based on the subset of cases and controls who had reported
their hair, eye and skin (freckling and skin sensitivity to sun)
pigmentation. Association within the individuals who are sensitive to sun,
and those who are not, is also shown. Adjustment for pigmentation was
done by including pigmentation characteristics as factor covariates in
the logistic regression estimating the OR.

| | | | | | |
|---|---|---|---|---|---|
| Gene | Variant | N case | N contr | OR (95% CI) | P |

Not adjusted for pigmentation characteristics

| | | | | | |
|---|---|---|---|---|---|
| ASIP | AH | 564 | 5,794 | 1.27 (1.02, 1.57) | 0.030 |
| TYR | rs1126809 A | 564 | 5,794 | 1.18 (1.03, 1.34) | 0.016 |
| TYRP1 | rs1408799 C | 564 | 5,794 | 1.21 (1.05, 1.40) | 0.0090 |
| MC1R | RHC | 558 | 4,147 | 1.03 (0.89, 1.18) | 0.72 |
| MC1R | NRHC | 558 | 4,147 | 1.09 (0.95, 1.26) | 0.23 |

Adjusted for hair, eye and skin pigmentation

| | | | | | |
|---|---|---|---|---|---|
| ASIP | AH | 564 | 5,794 | 1.21 (0.97, 1.50) | 0.088 |
| TYR | rs1126809 A | 564 | 5,794 | 1.19 (1.04, 1.35) | 0.013 |
| TYRP1 | rs1408799 C | 564 | 5,794 | 1.22 (1.05, 1.41) | 0.0086 |
| MC1R | RHC | 558 | 4,147 | 0.93 (0.79, 1.10) | 0.43 |
| MC1R | NRHC | 558 | 4,147 | 1.04 (0.89, 1.22) | 0.63 |

Stratified on skin sensitivity to sun:
Individuals sensitive to sun, adjusted for hair and eye pigmentation and
freckling

| | | | | | |
|---|---|---|---|---|---|
| ASIP | AH | 225 | 2,227 | 1.01 (0.74, 1.38) | 0.95 |
| TYR | rs1126809 A | 225 | 2,227 | 1.10 (0.90, 1.35) | 0.36 |
| TYRP1 | rs1408799 C | 225 | 2,227 | 1.27 (1.00, 1.61) | 0.053 |
| MC1R | RHC | 224 | 1,579 | 1.02 (0.81, 1.30) | 0.84 |
| MC1R | NRHC | 224 | 1,579 | 1.05 (0.81, 1.36) | 0.72 |

Individuals not sensitive to sun, adjusted for hair and eye pigmentation and
freckling

| | | | | | |
|---|---|---|---|---|---|
| ASIP | AH | 339 | 3,567 | 1.43 (1.06, 1.93) | 0.021 |
| TYR | rs1126809 A | 339 | 3,567 | 1.23 (1.03, 1.47) | 0.019 |
| TYRP1 | rs1408799 C | 339 | 3,567 | 1.18 (0.98, 1.42) | 0.075 |
| MC1R | RHC | 334 | 2,568 | 0.86 (0.68, 1.08) | 0.18 |
| MC1R | NRHC | 334 | 2,568 | 1.04 (0.85, 1.28) | 0.67 |

TABLE 24
The effect of the variants associating with skin cancer on age at diagnosis (AAD)
measured in years.

| | | CM (N = 2,010) | | BCC (N = 2,116) | |
|---|---|---|---|---|---|
| | | Effect on AAD | | Effect on AAD | |
| Locus | Variant | (95% CI) | P | (95% CI) | P |
| ASIP | AH | −2.00 (−3.80, −0.20) | 0.029 | 0.08 (1.69, 1.85) | 0.93 |
| TYR | rs1126809 A | −0.90 (−2.02, 0.21) | 0.11 | 0.71 (−0.11, 1.52) | 0.091 |
| TYRP1 | rs1408799 C | −3.99 (−9.21, 1.24) | 0.13 | −0,53 (−1.94, 0.88) | 0.46 |
| MC1R | RHC | −0.35 (−1.70, 1.00) | 0.61 | 0.46 (−0.52, 1.43) | 0.36 |
| MC1R | Other | −0.58 (−1.87, 0.71) | 0.38 | 0.80 (−0.15, 1.75) | 0.099 |

TABLE 25
Surrogate SNPs in linkage disequilibrium (LD) with rs1126809. The markers were
selected from the Caucasian HapMap dataset, using a cutoff of r² greater than
0.2. Shown are marker names, risk allele, values for D′ and r2 for the LD between
the anchor marker and the surrogate, the corresponding P-value, position of the marker
in NCBI Build 36 of the human genome assembly, and the identity of the SEQ ID for the
flanking sequence of the marker.

| | | | | | Pos. in | |
|---|---|---|---|---|---|---|
| SNP | Allele | D′ | r² | P-value | Build 36 | Seq ID No |
| rs3913310 | 4 | 0.724560 | 0.361726 | 2.20E−04 | 88162391 | 400 |
| rs17184781 | 1 | 0.687913 | 0.354719 | 2.93E−04 | 88202679 | 401 |
| rs7120151 | 1 | 0.822428 | 0.423777 | 4.95E−06 | 88380027 | 402 |
| rs7126679 | 4 | 0.658886 | 0.204189 | 0.00001974 | 88393493 | 403 |
| rs11018434 | 3 | 0.921504 | 0.229627 | 2.50E−03 | 88405427 | 404 |
| rs17791976 | 1 | 0.830520 | 0.509825 | 1.85E−09 | 88408490 | 405 |
| rs7931721 | 2 | 0.868513 | 0.509055 | 7.27E−10 | 88419424 | 406 |
| rs11018440 | 1 | 0.830520 | 0.509825 | 1.85E−09 | 88426718 | 407 |
| rs11018441 | 2 | 0.868495 | 0.536616 | 3.26E−10 | 88426947 | 408 |
| rs10830204 | 3 | 0.921826 | 0.233423 | 2.29E−03 | 88427192 | 409 |
| rs11018449 | 4 | 0.818006 | 0.500817 | 3.51E−08 | 88437034 | 410 |
| rs477424 | 2 | 0.892682 | 0.357262 | 7.93E−07 | 88441929 | 411 |
| rs7929744 | 1 | 0.917062 | 0.229048 | 4.85E−03 | 88444332 | 412 |
| rs7127487 | 3 | 0.845652 | 0.361820 | 2.90E−06 | 88454518 | 413 |
| rs10830206 | 1 | 0.854282 | 0.401789 | 1.71E−07 | 88455785 | 414 |
| rs4121738 | 4 | 0.849637 | 0.368415 | 8.85E−07 | 88456186 | 415 |
| rs11018463 | 2 | 0.830520 | 0.509825 | 1.85E−09 | 88459390 | 416 |
| rs11018464 | 2 | 0.826170 | 0.501063 | 4.01E−09 | 88460762 | 417 |
| rs3921012 | 1 | 0.854272 | 0.392898 | 1.79E−07 | 88465991 | 418 |
| rs7944714 | 2 | 0.848364 | 0.366287 | 1.31E−06 | 88470143 | 419 |
| rs10765186 | 1 | 0.915810 | 0.214669 | 1.04E−02 | 88470985 | 420 |
| rs9665831 | 4 | 0.849496 | 0.382759 | 4.71E−07 | 88473805 | 421 |
| rs1942497 | 2 | 0.814770 | 0.418476 | 1.66E−07 | 88481107 | 422 |
| rs2156123 | 4 | 0.849637 | 0.368415 | 8.85E−07 | 88488507 | 423 |
| rs7930256 | 2 | 0.810858 | 0.384330 | 1.23E−06 | 88489082 | 424 |
| rs4420272 | 4 | 0.810858 | 0.384330 | 1.23E−06 | 88490030 | 425 |
| rs7480884 | 3 | 0.814388 | 0.419194 | 3.98E−07 | 88491615 | 426 |
| rs12363323 | 1 | 0.830520 | 0.509825 | 1.85E−09 | 88495940 | 427 |
| rs1942486 | 3 | 0.826170 | 0.501063 | 4.01E−09 | 88496430 | 428 |
| rs10830216 | 1 | 0.854272 | 0.392898 | 1.79E−07 | 88498045 | 429 |
| rs17792911 | 4 | 0.826738 | 0.503023 | 8.76E−09 | 88502470 | 430 |
| rs4121729 | 1 | 0.849531 | 0.376225 | 8.86E−07 | 88502788 | 431 |
| rs10830219 | 4 | 0.828009 | 0.491391 | 5.74E−09 | 88512157 | 432 |
| rs10830228 | 1 | 0.750759 | 0.202350 | 1.90E−02 | 88530762 | 433 |
| rs10830231 | 2 | 0.768089 | 0.233445 | 2.29E−03 | 88535036 | 434 |
| rs7127661 | 4 | 0.759734 | 0.217494 | 6.76E−03 | 88536257 | 435 |
| rs10830236 | 4 | 0.833463 | 0.562047 | 1.02E−10 | 88540464 | 436 |
| rs949537 | 4 | 0.759734 | 0.217494 | 6.76E−03 | 88542478 | 437 |
| rs5021654 | 2 | 0.850822 | 0.389731 | 1.40E−06 | 88550237 | 438 |
| rs12270717 | 2 | 0.920201 | 0.785575 | 1.36E−16 | 88551838 | 439 |
| rs621313 | 2 | 0.785584 | 0.200154 | 8.87E−02 | 88553311 | 440 |
| rs7129973 | 3 | 0.855682 | 0.416296 | 2.56E−07 | 88555218 | 441 |
| rs11018525 | 2 | 0.854577 | 0.414306 | 3.80E−07 | 88559553 | 442 |
| rs17793678 | 4 | 1.000.000 | 0.857143 | 6.01E−27 | 88561172 | 443 |
| rs594647 | 1 | 0.832942 | 0.340472 | 4.73E−05 | 88561205 | 444 |
| rs10765196 | 2 | 1.000.000 | 0.857143 | 6.01E−27 | 88564890 | 445 |
| rs10765197 | 2 | 0.854577 | 0.414306 | 3.80E−07 | 88564976 | 446 |
| rs7123654 | 2 | 0.853427 | 0.412251 | 5.65E−07 | 88565603 | 447 |
| rs11018528 | 3 | 1.000.000 | 0.857498 | 4.08E−27 | 88570025 | 448 |
| rs12791412 | 3 | 0.920365 | 0.785855 | 3.35E−17 | 88570229 | 449 |
| rs12789914 | 1 | 0.839497 | 0.653823 | 1.54E−13 | 88570555 | 450 |
| rs7107143 | 4 | 0.850330 | 0.723060 | 9.95E−16 | 88571135 | 451 |
| rs574028 | 1 | 0.803680 | 0.230679 | 1.12E−02 | 88572898 | 452 |
| rs2000553 | 3 | 0.853292 | 0.402779 | 6.07E−07 | 88575655 | 453 |
| rs11018541 | 1 | 0.850822 | 0.389731 | 1.40E−06 | 88599795 | 454 |
| rs10765198 | 2 | 1.000.000 | 0.841176 | 9.67E−24 | 88609422 | 455 |
| rs7358418 | 3 | 0.883757 | 0.752521 | 6.32E−16 | 88609786 | 456 |
| rs10765200 | 4 | 0.917355 | 0.778650 | 3.10E−16 | 88611332 | 457 |
| rs10765201 | 2 | 0.917635 | 0.779301 | 2.10E−16 | 88611352 | 458 |
| rs4396293 | 2 | 0.899746 | 0.462778 | 4.57E−08 | 88615761 | 459 |
| rs2186640 | 3 | 0.850822 | 0.389731 | 1.40E−06 | 88615811 | 460 |
| rs10501698 | 1 | 1.000.000 | 0.755781 | 8.13E−23 | 88617012 | 461 |
| rs10830250 | 3 | 0.863763 | 0.542609 | 5.14E−09 | 88617255 | 462 |
| rs7924589 | 1 | 0.850560 | 0.585937 | 1.02E−08 | 88617956 | 463 |
| rs4121401 | 2 | 1.000.000 | 0.553186 | 5.98E−20 | 88619494 | 464 |
| rs10741305 | 2 | 1.000.000 | 0.311883 | 1.82E−11 | 88622366 | 465 |
| rs591260 | 3 | 1.000.000 | 0.309211 | 5.46E−12 | 88642214 | 466 |
| rs1847134 | 2 | 1.000.000 | 0.929093 | 6.35E−31 | 88644901 | 467 |
| rs1393350 | 1 | 1.000.000 | 0.857498 | 4.08E−27 | 88650694 | 16 |
| rs1126809 | 1 | 1 | 1 | NA | 88657609 | 137 |
| rs1827430 | 3 | 1.000.000 | 0.546603 | 2.45E−19 | 88658088 | 469 |
| rs3900053 | 2 | 0.949500 | 0.858727 | 1.33E−15 | 88660713 | 470 |
| rs1847142 | 1 | 1.000.000 | 0.963291 | 6.40E−32 | 88661222 | 471 |
| rs501301 | 3 | 1.000.000 | 0.290039 | 2.02E−11 | 88662321 | 472 |
| rs4121403 | 3 | 0.960259 | 0.855459 | 2.64E−18 | 88664103 | 473 |
| rs10830253 | 3 | 1.000.000 | 0.964389 | 4.26E−33 | 88667691 | 474 |
| rs7951935 | 4 | 1.000.000 | 0.561899 | 2.08E−19 | 88670047 | 475 |
| rs1502259 | 1 | 1.000.000 | 0.347015 | 2.24E−12 | 88675893 | 476 |
| rs1847140 | 3 | 0.960259 | 0.855459 | 2.64E−18 | 88676712 | 477 |
| rs1806319 | 3 | 1.000.000 | 0.618456 | 7.03E−22 | 88677584 | 478 |
| rs4106039 | 1 | 0.863151 | 0.558773 | 1.97E−08 | 88680791 | 479 |
| rs4106040 | 4 | 0.851523 | 0.534815 | 1.19E−06 | 88680802 | 480 |
| rs10830256 | 4 | 1.000.000 | 0.318647 | 1.90E−12 | 88685204 | 481 |
| rs3793973 | 1 | 0.593237 | 0.215068 | 0.00002612 | 88735642 | 482 |
| rs1847137 | 2 | 0.597106 | 0.213496 | 0.00002181 | 88736445 | 483 |

TABLE 26
Surrogate SNPs in linkage disequilibrium (LD) with rs1408799. The markers were
selected from the Caucasian HapMap dataset, using a cutoff of r² greater than
0.2. Shown are marker names, risk allele, values for D′ and r2 for the LD between
the anchor marker and the surrogate, the corresponding P-value, position of the marker
in NCBI Build 36 of the human genome assembly, and the identity of the SEQ ID for the
flanking sequence of the marker.

| SNP | Allele | D′ | r² | P-value | Pos. in | Seq ID No |
|---|---|---|---|---|---|---|
| rs791675 | 1 | 0.648373 | 0.235602 | 1.41E−03 | 12509087 | 222 |
| rs1325131 | 4 | 0.642632 | 0.223742 | 2.86E−03 | 12512752 | 223 |
| rs10756375 | 4 | 0.642632 | 0.223742 | 2.86E−03 | 12513291 | 224 |
| rs1590487 | 1 | 0.600000 | 0.221053 | 6.62E−03 | 12514085 | 225 |
| rs791691 | 2 | 0.603747 | 0.211355 | 7.30E−03 | 12517911 | 226 |
| rs791696 | 1 | 0.608302 | 0.219994 | 4.62E−03 | 12520255 | 227 |
| rs791697 | 3 | 0.603747 | 0.211355 | 7.30E−03 | 12520324 | 228 |
| rs702132 | 4 | 0.648373 | 0.235602 | 1.41E−03 | 12522047 | 229 |
| rs702133 | 4 | 0.623485 | 0.228389 | 6.20E−03 | 12522274 | 230 |
| rs702134 | 2 | 0.648373 | 0.235602 | 1.41E−03 | 12522458 | 231 |
| rs10960708 | 3 | 0.640919 | 0.238182 | 4.42E−03 | 12568438 | 232 |
| rs10809797 | 2 | 0.887455 | 0.223269 | 8.21E−03 | 12571270 | 233 |
| rs10429629 | 3 | 0.662589 | 0.376306 | 1.03E−06 | 12572787 | 234 |
| rs10960710 | 4 | 0.662589 | 0.376306 | 1.03E−06 | 12577153 | 235 |
| rs1022901 | 1 | 0.934506 | 0.457443 | 2.99E−09 | 12578259 | 3 |
| rs962298 | 4 | 0.486357 | 0.218207 | 2.31E−02 | 12578950 | 236 |
| rs6474717 | 3 | 0.701164 | 0.405981 | 1.34E−07 | 12579068 | 237 |
| rs1325112 | 4 | 0.895779 | 0.247287 | 1.86E−03 | 12582912 | 238 |
| rs1325113 | 2 | 1.000.000 | 0.212121 | 2.92E−03 | 12583080 | 239 |
| rs4428755 | 1 | 1.000.000 | 0.482759 | 9.66E−11 | 12583124 | 240 |
| rs10756380 | 1 | 1.000.000 | 0.212121 | 2.92E−03 | 12584967 | 241 |
| rs10756384 | 3 | 1.000.000 | 0.235474 | 6.48E−04 | 12586589 | 242 |
| rs13283146 | 4 | 0.739644 | 0.501402 | 4.51E−09 | 12589561 | 243 |
| rs1408790 | 3 | 0.836610 | 0.647290 | 1.41E−14 | 12592681 | 244 |
| rs1408791 | 3 | 1.000.000 | 0.259259 | 1.40E−04 | 12592864 | 245 |
| rs10960716 | 4 | 0.836610 | 0.647290 | 1.41E−14 | 12594407 | 246 |
| rs713596 | 2 | 0.875963 | 0.682992 | 6.95E−16 | 12595687 | 247 |
| rs1325115 | 4 | 1.000.000 | 0.283489 | 2.93E−05 | 12598182 | 248 |
| rs1325116 | 4 | 1.000.000 | 0.235474 | 6.48E−04 | 12598432 | 249 |
| rs1408792 | 2 | 1.000.000 | 0.218750 | 2.27E−03 | 12599014 | 250 |
| rs10809806 | 3 | 0.700026 | 0.364386 | 5.72E−06 | 12601123 | 251 |
| rs13288558 | 3 | 0.875963 | 0.682992 | 6.95E−16 | 12602529 | 252 |
| rs2025556 | 2 | 1.000.000 | 0.212121 | 2.92E−03 | 12603216 | 253 |
| rs1325117 | 1 | 0.839441 | 0.639061 | 5.46E−10 | 12603472 | 254 |
| rs6474718 | 2 | 0.697835 | 0.362108 | 1.11E−05 | 12604387 | 255 |
| rs13283649 | 1 | 0.916123 | 0.715445 | 4.82E−17 | 12608337 | 256 |
| rs1325118 | 4 | 0.753565 | 0.545592 | 3.28E−11 | 12609616 | 257 |
| rs10738286 | 4 | 1.000.000 | 0.225806 | 1.75E−03 | 12609795 | 258 |
| rs7466934 | 4 | 0.916287 | 0.719642 | 2.45E−17 | 12609840 | 259 |
| rs10960721 | 3 | 0.634009 | 0.234481 | 7.47E−03 | 12610116 | 260 |
| rs7036899 | 3 | 0.916287 | 0.719642 | 2.45E−17 | 12610266 | 261 |
| rs10756386 | 3 | 0.916287 | 0.719642 | 2.45E−17 | 12611004 | 262 |
| rs10960723 | 1 | 0.871971 | 0.675227 | 7.55E−15 | 12612878 | 263 |
| rs4612469 | 3 | 1.000.000 | 0.212121 | 2.92E−03 | 12612925 | 264 |
| rs977888 | 1 | 0.916287 | 0.719642 | 2.45E−17 | 12614357 | 265 |
| rs10809808 | 3 | 0.875963 | 0.682992 | 6.95E−16 | 12614463 | 7 |
| rs10756387 | 1 | 1.000.000 | 0.212121 | 2.92E−03 | 12618599 | 266 |
| rs10960730 | 3 | 0.875963 | 0.682992 | 6.95E−16 | 12621099 | 267 |
| rs10809809 | 3 | 0.875845 | 0.680931 | 9.62E−16 | 12621398 | 268 |
| rs10125059 | 4 | 1.000.000 | 0.212121 | 2.92E−03 | 12621525 | 269 |
| rs10756388 | 1 | 1.000.000 | 0.358974 | 2.20E−07 | 12622930 | 270 |
| rs10960731 | 2 | 1.000.000 | 0.235474 | 6.48E−04 | 12623322 | 271 |
| rs10960732 | 1 | 0.875963 | 0.682992 | 6.95E−16 | 12623495 | 272 |
| rs7026116 | 1 | 0.874987 | 0.675722 | 7.44E−15 | 12623981 | 273 |
| rs10124166 | 1 | 1.000.000 | 0.212121 | 2.92E−03 | 12627846 | 274 |
| rs7047297 | 1 | 0.914929 | 0.691259 | 3.04E−16 | 12628540 | 275 |
| rs13301970 | 2 | 0.724015 | 0.439308 | 2.27E−07 | 12629877 | 276 |
| rs10960735 | 2 | 0.953310 | 0.746851 | 2.85E−16 | 12631821 | 277 |
| rs1325122 | 4 | 0.916096 | 0.719342 | 4.91E−17 | 12632878 | 278 |
| rs6474720 | 1 | 1.000.000 | 0.235474 | 6.48E−04 | 12633558 | 279 |
| rs6474721 | 1 | 1.000.000 | 0.225806 | 1.75E−03 | 12633660 | 280 |
| rs10960738 | 2 | 0.771325 | 0.524202 | 8.40E−09 | 12638831 | 281 |
| rs13283345 | 1 | 0.764774 | 0.494753 | 6.05E−09 | 12640198 | 282 |
| rs10809811 | 3 | 0.957431 | 0.756975 | 2.07E−18 | 12640996 | 283 |
| rs1408794 | 3 | 0.957526 | 0.757125 | 1.03E−18 | 12641340 | 284 |
| rs1408795 | 4 | 0.734246 | 0.478611 | 6.95E−09 | 12641413 | 285 |
| rs13294940 | 4 | 1.000.000 | 0.636364 | 2.95E−15 | 12642364 | 286 |
| rs1325124 | 2 | 1.000.000 | 0.259259 | 1.40E−04 | 12642651 | 287 |
| rs996697 | 1 | 1.000.000 | 0.466667 | 1.86E−10 | 12642983 | 288 |
| rs2382359 | 2 | 1.000.000 | 0.397993 | 2.41E−08 | 12643846 | 289 |
| rs995263 | 4 | 0.916096 | 0.719342 | 4.91E−17 | 12644578 | 290 |
| rs1325125 | 1 | 1.000.000 | 0.340278 | 1.76E−06 | 12645862 | 291 |
| rs10435754 | 4 | 0.625407 | 0.222029 | 1.92E−02 | 12647603 | 292 |
| rs4741242 | 1 | 1.000.000 | 0.259259 | 1.40E−04 | 12649691 | 293 |
| rs2209275 | 3 | 1.000.000 | 0.553265 | 5.41E−13 | 12653234 | 294 |
| rs7022317 | 3 | 0.697118 | 0.362572 | 7.04E−06 | 12656686 | 295 |
| rs1121541 | 1 | 0.957526 | 0.757125 | 1.03E−18 | 12657049 | 296 |
| rs10809818 | 3 | 0.760669 | 0.578617 | 2.94E−12 | 12658121 | 297 |
| rs1325127 | 4 | 0.760669 | 0.578617 | 2.94E−12 | 12658328 | 298 |
| rs10960748 | 2 | 0.957619 | 0.757273 | 5.14E−19 | 12658805 | 299 |
| rs9298679 | 1 | 1.000.000 | 0.677419 | 8.55E−17 | 12659346 | 300 |
| rs9298680 | 4 | 1.000.000 | 0.283489 | 2.93E−05 | 12659377 | 301 |
| rs7863161 | 2 | 1.000.000 | 0.283489 | 2.93E−05 | 12659735 | 302 |
| rs1041105 | 1 | 1.000.000 | 0.283489 | 2.93E−05 | 12661059 | 303 |
| rs10960749 | 1 | 1.000.000 | 0.795918 | 3.30E−22 | 12661566 | 304 |
| rs1408799 | 2 | 1 | 1 | NA | 12662097 | 17 |
| rs1408800 | 1 | 1.000.000 | 1.000.000 | 9.56E−30 | 12662275 | 305 |
| rs13294134 | 3 | 1.000.000 | 0.795918 | 3.30E−22 | 12663636 | 306 |
| rs16929340 | 4 | 0.624392 | 0.227422 | 2.68E−02 | 12664124 | 307 |
| rs13299830 | 3 | 0.675767 | 0.318166 | 3.02E−04 | 12664531 | 308 |
| rs10960751 | 2 | 0.956376 | 0.754088 | 2.09E−18 | 12665264 | 309 |
| rs10960752 | 1 | 0.956376 | 0.754088 | 2.09E−18 | 12665284 | 310 |
| rs10960753 | 4 | 0.913657 | 0.715117 | 1.97E−16 | 12665522 | 311 |
| rs16929342 | 4 | 1.000.000 | 0.212121 | 2.92E−03 | 12665661 | 312 |
| rs16929345 | 3 | 1.000.000 | 0.235474 | 6.48E−04 | 12666236 | 313 |
| rs16929346 | 2 | 1.000.000 | 0.308176 | 5.94E−06 | 12666417 | 314 |
| rs13296454 | 1 | 0.957042 | 0.729004 | 3.30E−18 | 12667181 | 315 |
| rs13297008 | 1 | 0.957042 | 0.729004 | 3.30E−18 | 12667471 | 316 |
| rs10116013 | 1 | 0.603788 | 0.230664 | 9.64E−03 | 12667979 | 317 |
| rs10809826 | 3 | 0.957619 | 0.757273 | 5.14E−19 | 12672663 | 318 |
| rs7847593 | 1 | 1.000.000 | 0.212121 | 2.92E−03 | 12673639 | 319 |
| rs13293905 | 1 | 0.693875 | 0.426122 | 9.57E−08 | 12675943 | 320 |
| rs2762460 | 2 | 0.911065 | 0.652387 | 1.36E−14 | 12686478 | 321 |
| rs2762461 | 4 | 0.956448 | 0.702050 | 1.88E−17 | 12686499 | 322 |
| rs2762462 | 2 | 0.741293 | 0.390236 | 6.21E−07 | 12689776 | 323 |
| rs2762463 | 4 | 0.660379 | 0.402294 | 3.85E−07 | 12691897 | 324 |
| rs2224863 | 1 | 0.639606 | 0.409096 | 2.05E−07 | 12692890 | 325 |
| rs2733830 | 4 | 0.692221 | 0.425658 | 8.64E−08 | 12693359 | 326 |
| rs2733831 | 3 | 0.913996 | 0.664902 | 8.33E−16 | 12693484 | 327 |
| rs2733832 | 4 | 0.955837 | 0.676320 | 9.66E−17 | 12694725 | 328 |
| rs2733833 | 3 | 0.628783 | 0.379863 | 1.41E−06 | 12695095 | 329 |
| rs2209277 | 2 | 0.660379 | 0.402294 | 3.85E−07 | 12696236 | 330 |
| rs2733834 | 3 | 0.624910 | 0.375198 | 5.28E−06 | 12698910 | 331 |
| rs683 | 1 | 0.634841 | 0.387462 | 7.16E−07 | 12699305 | 332 |
| rs2762464 | 4 | 0.639606 | 0.409096 | 2.05E−07 | 12699586 | 333 |
| rs910 | 2 | 0.704556 | 0.457919 | 6.56E−09 | 12700035 | 334 |
| rs1063380 | 2 | 0.704556 | 0.457919 | 6.56E−09 | 12700090 | 335 |
| rs9298681 | 4 | 0.647146 | 0.205769 | 6.38E−02 | 12701032 | 336 |
| rs10960758 | 1 | 0.953966 | 0.709186 | 1.48E−15 | 12706315 | 337 |
| rs10960759 | 1 | 0.957153 | 0.729175 | 1.64E−18 | 12706428 | 338 |
| rs12379024 | 1 | 0.957153 | 0.729175 | 1.64E−18 | 12707405 | 339 |
| rs13295868 | 3 | 0.957153 | 0.729175 | 1.64E−18 | 12707912 | 340 |
| rs7019226 | 4 | 0.956579 | 0.702242 | 9.36E−18 | 12708370 | 341 |
| rs11789751 | 3 | 0.956137 | 0.747384 | 1.19E−17 | 12709264 | 342 |
| rs10491744 | 2 | 0.957153 | 0.729175 | 1.64E−18 | 12710106 | 343 |
| rs10960760 | 3 | 0.957153 | 0.729175 | 1.64E−18 | 12710152 | 344 |
| rs2382361 | 2 | 0.957153 | 0.729175 | 1.64E−18 | 12710786 | 345 |
| rs1409626 | 1 | 0.957153 | 0.729175 | 1.64E−18 | 12710820 | 346 |
| rs1409630 | 2 | 0.956579 | 0.702242 | 9.36E−18 | 12711251 | 347 |
| rs13288475 | 4 | 0.956579 | 0.702242 | 9.36E−18 | 12711714 | 348 |
| rs13288636 | 1 | 0.956579 | 0.702242 | 9.36E−18 | 12711806 | 349 |
| rs13288681 | 2 | 0.956450 | 0.697100 | 2.99E−17 | 12711881 | 350 |
| rs1326798 | 3 | 0.956579 | 0.702242 | 9.36E−18 | 12712227 | 351 |
| rs7871257 | 1 | 0.606924 | 0.238732 | 7.28E−03 | 12712357 | 352 |
| rs12379260 | 1 | 0.956448 | 0.702050 | 1.88E−17 | 12713112 | 353 |
| rs13284453 | 4 | 0.954016 | 0.633960 | 1.17E−14 | 12714280 | 354 |
| rs13284898 | 4 | 0.956205 | 0.691607 | 7.76E−17 | 12714560 | 355 |
| rs7048117 | 3 | 0.761714 | 0.338455 | 1.13E−05 | 12725950 | 356 |
| rs10756400 | 3 | 0.759278 | 0.481688 | 2.62E−07 | 12728157 | 357 |
| rs970944 | 2 | 0.776746 | 0.511920 | 1.29E−10 | 12728401 | 358 |
| rs970945 | 2 | 0.776746 | 0.511920 | 1.29E−10 | 12728641 | 359 |
| rs970946 | 2 | 0.776746 | 0.511920 | 1.29E−10 | 12728690 | 360 |
| rs970947 | 1 | 0.776746 | 0.511920 | 1.29E−10 | 12728813 | 361 |
| rs10960774 | 3 | 0.957042 | 0.729004 | 3.30E−18 | 12729313 | 362 |
| rs10756402 | 1 | 0.751033 | 0.463601 | 1.98E−07 | 12729948 | 363 |
| rs10756403 | 4 | 0.679687 | 0.304219 | 1.83E−03 | 12730760 | 364 |
| rs10738290 | 1 | 0.713992 | 0.313026 | 5.33E−05 | 12730906 | 365 |
| rs13300005 | 1 | 0.902955 | 0.271776 | 4.08E−04 | 12738191 | 366 |
| rs10756406 | 4 | 0.957713 | 0.757420 | 2.56E−19 | 12738587 | 367 |
| rs7019486 | 2 | 0.760092 | 0.349742 | 1.05E−05 | 12738633 | 368 |
| rs927868 | 4 | 0.912094 | 0.649295 | 1.47E−14 | 12738795 | 369 |
| rs7019981 | 3 | 0.761714 | 0.338455 | 1.13E−05 | 12738818 | 370 |
| rs927869 | 3 | 0.957713 | 0.757420 | 2.56E−19 | 12738962 | 123 |
| rs4741245 | 3 | 0.957713 | 0.757420 | 2.56E−19 | 12739300 | 371 |
| rs7023927 | 1 | 0.957713 | 0.757420 | 2.56E−19 | 12739596 | 372 |
| rs7035500 | 4 | 0.957632 | 0.753199 | 5.16E−19 | 12740095 | 373 |
| rs13302551 | 4 | 0.957153 | 0.729175 | 1.64E−18 | 12740812 | 374 |
| rs1543587 | 2 | 0.957713 | 0.757420 | 2.56E−19 | 12741741 | 375 |
| rs1074789 | 3 | 0.916477 | 0.719940 | 1.22E−17 | 12742340 | 376 |
| rs2181816 | 2 | 0.761714 | 0.338455 | 1.13E−05 | 12742760 | 377 |
| rs10125771 | 3 | 0.709994 | 0.235243 | 5.04E−03 | 12747058 | 378 |
| rs10960779 | 3 | 0.916477 | 0.719940 | 1.22E−17 | 12748881 | 379 |
| rs1326789 | 1 | 0.907504 | 0.682571 | 4.84E−15 | 12749838 | 380 |
| rs7025842 | 1 | 0.915384 | 0.691948 | 7.56E−17 | 12750647 | 381 |
| rs7025953 | 1 | 0.915384 | 0.691948 | 7.56E−17 | 12750718 | 382 |
| rs7025771 | 4 | 0.915384 | 0.691948 | 7.56E−17 | 12750762 | 383 |
| rs7025914 | 4 | 0.914263 | 0.665289 | 4.16E−16 | 12750884 | 384 |
| rs10491743 | 1 | 0.915384 | 0.691948 | 7.56E−17 | 12750920 | 385 |
| rs1326790 | 1 | 0.915384 | 0.691948 | 7.56E−17 | 12751168 | 386 |
| rs1326791 | 2 | 0.908647 | 0.708641 | 1.72E−15 | 12751300 | 387 |
| rs1326792 | 2 | 0.915384 | 0.691948 | 7.56E−17 | 12751360 | 388 |
| rs7030485 | 1 | 0.909865 | 0.673504 | 2.59E−14 | 12751819 | 389 |
| rs10960781 | 1 | 0.876138 | 0.681389 | 4.80E−16 | 12752374 | 390 |
| rs12115198 | 2 | 0.877720 | 0.710816 | 6.97E−17 | 12753450 | 391 |
| rs10960783 | 1 | 0.864140 | 0.659945 | 5.08E−13 | 12753809 | 392 |
| rs1041176 | 2 | 0.873603 | 0.422242 | 8.01E−08 | 12754311 | 393 |
| rs10119113 | 3 | 0.818287 | 0.390597 | 6.57E−07 | 12755117 | 394 |
| rs1326795 | 4 | 0.902955 | 0.271776 | 4.08E−04 | 12760108 | 395 |
| rs2209273 | 2 | 0.831933 | 0.266542 | 4.08E−04 | 12762498 | 396 |
| rs7855624 | 2 | 0.950398 | 0.671652 | 2.29E−15 | 12763263 | 397 |
| rs10491742 | 4 | 0.837002 | 0.647898 | 7.08E−15 | 12765488 | 398 |
| rs3750502 | 2 | 0.928555 | 0.402367 | 1.12E−07 | 12766516 | 399 |

1. A method of determining a susceptibility to a skin cancer in a human individual, the method comprising
(a) determining the presence or absence of at least one allele of at least one polymorphic marker in a nucleic acid sample obtained from the individual, or in a genotype dataset from the individual, wherein the at least one polymorphic marker is associated with at least one gene selected from the ASIP gene, the TYR gene and the TYRP1 gene, and
(b) determine a susceptibility to the skin cancer based on the presence of the at least one allele of the at least one polymorphic marker.

2. The method according to claim 1, wherein the skin cancer is selected from the group consisting of melanoma, basal cell carcinoma and squamous cell carcinoma.

3. The method according to claim 1, wherein the at least one marker is selected from the group consisting of marker rs1015362, rs4911414, rs1126809, rs1408799, rs6060043, and rs1393350, and markers in linkage disequilibrium therewith.

4. The method according to any one of the preceding claims, wherein the at least one marker is selected from the group consisting of rs1126809 and rs1408799, and markers in linkage disequilibrium therewith.

5. The method according to claim 4, wherein the markers in linkage disequilibrium with rs1126809 are selected from the group consisting of rs3913310, rs17184781, rs7120151, rs7126679, rs11018434, rs17791976, rs7931721, rs11018440, rs11018441, rs10830204, rs11018449, rs477424, rs7929744, rs7127487, rs10830206, rs4121738, rs11018463, rs11018464, rs3921012, rs7944714, rs10765186, rs9665831, rs1942497, rs2156123, rs7930256, rs4420272, rs7480884, rs12363323, rs1942486, rs10830216, rs17792911, rs4121729, rs10830219, rs10830228, rs10830231, rs7127661, rs10830236, rs949537, rs5021654, rs12270717, rs621313, rs7129973, rs11018525, rs17793678, rs594647, rs10765196, rs10765197, rs7123654, rs11018528, rs12791412, rs12789914, rs7107143, rs574028, rs2000553, rs11018541, rs10765198, rs7358418, rs10765200, rs10765201, rs4396293, rs2186640, rs10501698, rs10830250, rs7924589, rs4121401, rs10741305, rs591260, rs1847134, rs1393350, rs1126809, rs1827430, rs3900053, rs1847142, rs501301, rs4121403, rs10830253, rs7951935, rs1502259, rs1847140, rs1806319, rs4106039, rs4106040, rs10830256, rs3793973 and rs1847137.

6. The method according to claim 4, wherein the markers in linkage disequilibrium with rs1408799 are selected from the group consisting of rs791675, rs1325131, rs10756375, rs1590487, rs791691, rs791696, rs791697, rs702132, rs702133, rs702134, rs10960708, rs10809797, rs10429629, rs10960710, rs1022901, rs962298, rs6474717, rs1325112, rs1325113, rs4428755, rs10756380, rs10756384, rs13283146, rs1408790, rs1408791, rs10960716, rs713596, rs1325115, rs1325116, rs1408792, rs10809806, rs13288558, rs2025556, rs1325117, rs6474718, rs13283649, rs1325118, rs10738286, rs7466934, rs10960721, rs7036899, rs10756386, rs10960723, rs4612469, rs977888, rs10809808, rs10756387, rs10960730, rs10809809, rs10125059, rs10756388, rs10960731, rs10960732, rs7026116, rs10124166, rs7047297, rs13301970, rs10960735, rs1325122, rs6474720, rs6474721, rs10960738, rs13283345, rs10809811, rs1408794, rs1408795, rs13294940, rs1325124, rs996697, rs2382359, rs995263, rs1325125, rs10435754, rs4741242, rs2209275, rs7022317, rs1121541, rs10809818, rs1325127, rs10960748, rs9298679, rs9298680, rs7863161, rs1041105, rs10960749, rs1408799, rs1408800, rs13294134, rs16929340, rs13299830, rs10960751, rs10960752, rs10960753, rs16929342, rs16929345, rs16929346, rs13296454, rs13297008, rs10116013, rs10809826, rs7847593, rs13293905, rs2762460, rs2762461, rs2762462, rs2762463, rs2224863, rs2733830, rs2733831, rs2733832, rs2733833, rs2209277, rs2733834, rs683, rs2762464, rs910, rs1063380, rs9298681, rs10960758, rs10960759, rs12379024, rs13295868, rs7019226, rs11789751, rs10491744, rs10960760, rs2382361, rs1409626, rs1409630, rs13288475, rs13288636, rs13288681, rs1326798, rs7871257, rs12379260, rs13284453, rs13284898, rs7048117, rs10756400, rs970944, rs970945, rs970946, rs970947, rs10960774, rs10756402, rs10756403, rs10738290, rs13300005, rs10756406, rs7019486, rs927868, rs7019981, rs927869, rs4741245, rs7023927, rs7035500, rs13302551, rs1543587, rs1074789, rs2181816, rs10125771, rs10960779, rs1326789, rs7025842, rs7025953, rs7025771, rs7025914, rs10491743, rs1326790, rs1326791, rs1326792, rs7030485, rs10960781, rs12115198, rs10960783, rs1041176, rs10119113, rs1326795, rs2209273, rs7855624, rs10491742, and rs3750502.

7. The method according to any one of the preceding claims, comprising determining whether at least one allele in each of at least two polymorphic markers is present in a nucleic acid sample obtained from the individual, or in a genotype dataset derived from the individual.

8. The method according to claim 7, further comprising whether at least one haplotype comprising at least two polymorphic markers is present in a nucleic acid sample obtained from the individual, or in a genotype dataset derived from the individual.

9. The method according to any one of the preceding claims, wherein the genotype dataset comprises genotype information from a preexisting record.

10. The method according to any one of the preceding claims, wherein determining a susceptibility to the skin cancer comprises comparing results of determination of the presence of the at least one allele of the at least one polymorphic marker or the at least one haplotype to a database containing correlation data between the at least one polymorphic marker or the at least one haplotype and susceptibility to the skin cancer.

11. The method according to claim 10, wherein the database comprises at least one measure of susceptibility to the skin cancer for the at least one polymorphic marker or haplotype.

12. The method according to claim 8, wherein the at least one haplotype comprises rs1015362 allele A and rs4911414 allele T.

13. The method according to claim 1, wherein the at least one polymorphic marker is a marker in linkage disequilibrium with the haplotype comprising rs1015362 allele A and rs4911414 allele T.

14. The method according to any one of claims 1-12 and 13, wherein the at least one polymorphic marker is selected from the group consisting of rs1885120, rs17401449, rs291671, rs291695, rs293721, rs721970, rs910873, rs17305573, rs4911442, rs1204552, rs293709, rs6058091, rs1884431, rs6142199, rs2068474, rs2378199, rs2378249, rs2425003, rs4302281, rs4564863, rs4911430, rs6059928, rs6059937, rs6059961, rs6059969, rs6087607, rs2144956, rs2295443, rs2889849, rs6058089, rs6059916, rs932542, rs17421899, rs1884432, rs7265992, rs17092148, rs3787220, rs3787223, rs6058115, rs6060009, rs6060017, rs6060030, rs6060034, rs6060043, rs6060047, rs6088594, rs7271289, rs910871, rs6088316, rs17396317, rs2425067, rs6058339, rs6060612, rs2378412, rs293738, rs1205339, rs2281695, rs4911154, rs6088515, rs7269526, rs17305657, rs1122174, rs6060025, rs6059908, rs4911523, rs4911315, rs619865, rs6059931, rs11546155, rs221981, rs17122844, rs7272741, rs2425020, rs2424941, rs761930, rs221984, rs2378078, rs2424944, rs633784, rs666210, rs7361656, rs2424948, rs2424994, rs221985, rs17092378, rs2050652, rs6058192, rs6059662, and rs7274811.

15. The method according to any one of the preceding claims, wherein the at least one allele or haplotype is predictive of an increased susceptibility to the skin cancer.

16. The method according to claim 15, wherein the increased susceptibility is characterized by a relative or an odds ratio of at least 1.05, including at least 1.10, at least 1.15, at least 1.20, at least 1.25, at least 1.30, at least 1.35, at least 1.40, at least 1.45 or at least 1.50.

17. The method according to claim 15 or claim 16, wherein the at least one allele or haplotype comprises at least one allele selected from the group consisting of rs1015362 allele G, rs4911414 allele T, rs1126809 allele A, rs1408799 allele C, rs6060043 allele C, and rs1393350 allele A.

18. A method of determining a susceptibility to a skin cancer in a human individual, comprising
(a) obtaining sequence data about a human individual, wherein the data includes identification of at least one allele of at least one polymorphic marker associated with at least one gene selected from the ASIP gene, the TYR gene and the TYRP1 gene, wherein different alleles of the at least one polymorphic marker are associated with different susceptibilities to the skin cancer in humans, and
(b) determining a susceptibility to the skin cancer from the sequence data of the individual.

19. The method according to claim 18, wherein the skin cancer is selected from the group consisting of melanoma, basal cell carcinoma and squamous cell carcinoma.

20. The method according to claim 18 or claim 19, wherein the sequence data is nucleic acid sequence data.

21. The method according to any one of the claims 18-20, wherein obtaining nucleic acid sequence data comprises analyzing sequence of the at least one polymorphic marker in a biological sample from the individual.

22. The method according to any one of the claims 18-21, wherein obtaining nucleic acid sequence data comprises determining the identity of at least one haplotype comprising at least two polymorphic markers.

23. The method according to any one of claims 18-22, wherein obtaining nucleic acid sequence data comprises obtaining a genotype dataset from the human individual and analyzing sequence of the at least one polymorphic marker in the dataset.

24. The method according to any one of claims 21-23, wherein analyzing sequence of at least one polymorphic marker comprises determining the presence or absence of at least one allele of the at least one polymorphic marker.

25. The method according to any of the claims 21-24, wherein the sequence data is amino acid sequence data.

26. The method according claim 25, wherein analyzing amino acid sequence data comprises determining the presence or absence of an amino acid substitution in the amino acid encoded by the at least one polymorphic marker.

27. The method according to claim 25 or claim 26, wherein obtaining amino acid sequence data comprises analyzing the amino acid sequence encoded by the at least one polymorphic marker in a biological sample obtained from the individual.

28. The method according to any of the claims 18-27, wherein obtaining sequence data comprises obtaining nucleic acid sequence information or amino acid sequence information from a preexisting record.

29. The method according to any one of the claims 18-28, wherein determining a susceptibility comprises comparing the sequence data to a database containing correlation data between the at least one polymorphic marker and susceptibility to the skin cancer.

30. The method according to claim 29, wherein the database comprises at least one measure of susceptibility to the skin cancer for the at least one polymorphic marker.

31. The method according to claim 29 or 30, wherein the database comprises a look-up table comprising at least one measure of susceptibility to the skin cancer for the at least one polymorphic marker.

32. The method according to any one of the claims 18-31, wherein the at least one marker is selected from the group consisting of rs1126809 and rs1408799, and markers in linkage disequilibrium therewith.

33. The method according to claim 32, wherein the markers in linkage disequilibrium with rs1126809 are selected from the group consisting of rs3913310, rs17184781, rs7120151, rs7126679, rs11018434, rs17791976, rs7931721, rs11018440, rs11018441, rs10830204, rs11018449, rs477424, rs7929744, rs7127487, rs10830206, rs4121738, rs11018463, rs11018464, rs3921012, rs7944714, rs10765186, rs9665831, rs1942497, rs2156123, rs7930256, rs4420272, rs7480884, rs12363323, rs1942486, rs10830216, rs17792911, rs4121729, rs10830219, rs10830228, rs10830231, rs7127661, rs10830236, rs949537, rs5021654, rs12270717, rs621313, rs7129973, rs11018525, rs17793678, rs594647, rs10765196, rs10765197, rs7123654, rs11018528, rs12791412, rs12789914, rs7107143, rs574028, rs2000553, rs11018541, rs10765198, rs7358418, rs10765200, rs10765201, rs4396293, rs2186640, rs10501698, rs10830250, rs7924589, rs4121401, rs10741305, rs591260, rs1847134, rs1393350, rs1126809, rs1827430, rs3900053, rs1847142, rs501301, rs4121403, rs10830253, rs7951935, rs1502259, rs1847140, rs1806319, rs4106039, rs4106040, rs10830256, rs3793973 and rs1847137.

34. The method according to claim 32, wherein the markers in linkage disequilibrium with rs1408799 are selected from the group consisting of rs791675, rs1325131, rs10756375, rs1590487, rs791691, rs791696, rs791697, rs702132, rs702133, rs702134, rs10960708, rs10809797, rs10429629, rs10960710, rs1022901, rs962298, rs6474717, rs1325112, rs1325113, rs4428755, rs10756380, rs10756384, rs13283146, rs1408790, rs1408791, rs10960716, rs713596, rs1325115, rs1325116, rs1408792, rs10809806, rs13288558, rs2025556, rs1325117, rs6474718, rs13283649, rs1325118, rs10738286, rs7466934, rs10960721, rs7036899, rs10756386, rs10960723, rs4612469, rs977888, rs10809808, rs10756387, rs10960730, rs10809809, rs10125059, rs10756388, rs10960731, rs10960732, rs7026116, rs10124166, rs7047297, rs13301970, rs10960735, rs1325122, rs6474720, rs6474721, rs10960738, rs13283345, rs10809811, rs1408794, rs1408795, rs13294940, rs1325124, rs996697, rs2382359, rs995263, rs1325125, rs10435754, rs4741242, rs2209275, rs7022317, rs1121541, rs10809818, rs1325127, rs10960748, rs9298679, rs9298680, rs7863161, rs1041105, rs10960749, rs1408799, rs1408800, rs13294134, rs16929340, rs13299830, rs10960751, rs10960752, rs10960753, rs16929342, rs16929345, rs16929346, rs13296454, rs13297008, rs10116013, rs10809826, rs7847593, rs13293905, rs2762460, rs2762461, rs2762462, rs2762463, rs2224863, rs2733830, rs2733831, rs2733832, rs2733833, rs2209277, rs2733834, rs683, rs2762464, rs910, rs1063380, rs9298681, rs10960758, rs10960759, rs12379024, rs13295868, rs7019226, rs11789751, rs10491744, rs10960760, rs2382361, rs1409626, rs1409630, rs13288475, rs13288636, rs13288681, rs1326798, rs7871257, rs12379260, rs13284453, rs13284898, rs7048117, rs10756400, rs970944, rs970945, rs970946, rs970947, rs10960774, rs10756402, rs10756403, rs10738290, rs13300005, rs10756406, rs7019486, rs927868, rs7019981, rs927869, rs4741245, rs7023927, rs7035500, rs13302551, rs1543587, rs1074789, rs2181816, rs10125771, rs10960779, rs1326789, rs7025842, rs7025953, rs7025771, rs7025914, rs10491743, rs1326790, rs1326791, rs1326792, rs7030485, rs10960781, rs12115198, rs10960783, rs1041176, rs10119113, rs1326795, rs2209273, rs7855624, rs10491742, and rs3750502.

35. The method according to any one of the claims 18-34, wherein the at least one allele or haplotype is predictive of an increased susceptibility to the skin cancer.

36. The method according to claim 35, wherein the increased susceptibility is characterized by a relative or an odds ratio of at least 1.05, including at least 1.10, at least 1.15, at least 1.20, at least 1.25, at least 1.30, at least 1.35, at least 1.40, at least 1.45 or at least 1.50.

37. The method according to claim 35 or claim 36, wherein the at least one allele or haplotype comprises at least one allele selected from the group consisting of rs1015362 allele G, rs4911414 allele T, rs1126809 allele A, rs1408799 allele C, rs6060043 allele C, and rs1393350 allele A.

38. The method according to any one of the claims 18-31, wherein the at least one polymorphic marker associated with the ASIP gene is a marker in linkage disequilibrium with the haplotype comprising rs1015362 allele A and rs4911414 allele T.

39. The method according to claim 38, wherein the at least one polymorphic marker is selected from the group consisting of rs1885120, rs17401449, rs291671, rs291695, rs293721, rs721970, rs910873, rs17305573, rs4911442, rs1204552, rs293709, rs6058091, rs1884431, rs6142199, rs2068474, rs2378199, rs2378249, rs2425003, rs4302281, rs4564863, rs4911430, rs6059928, rs6059937, rs6059961, rs6059969, rs6087607, rs2144956, rs2295443, rs2889849, rs6058089, rs6059916, rs932542, rs17421899, rs1884432, rs7265992, rs17092148, rs3787220, rs3787223, rs6058115, rs6060009, rs6060017, rs6060030, rs6060034, rs6060043, rs6060047, rs6088594, rs7271289, rs910871, rs6088316, rs17396317, rs2425067, rs6058339, rs6060612, rs2378412, rs293738, rs1205339, rs2281695, rs4911154, rs6088515, rs7269526, rs17305657, rs1122174, rs6060025, rs6059908, rs4911523, rs4911315, rs619865, rs6059931, rs11546155, rs221981, rs17122844, rs7272741, rs2425020, rs2424941, rs761930, rs221984, rs2378078, rs2424944, rs633784, rs666210, rs7361656, rs2424948, rs2424994, rs221985, rs17092378, rs2050652, rs6058192, rs6059662, and rs7274811.

40. A method of screening a candidate marker for assessing susceptibility to at least one skin cancer selected from the group consisting of melanoma, basal cell carcinoma and squamous cell carcinoma, comprising analyzing the frequency of at least one allele of a polymorphic marker associated with at least one of the ASIP gene, the TYR gene and the TYRP1 gene, in a population of human individuals diagnosed with the skin cancer, wherein a significant difference in frequency of the at least one allele in the population of human individuals diagnosed with the skin cancer as compared to the frequency of the at least one allele in a control population of human individuals is indicative of the marker as a susceptibility marker for the skin cancer.

41. A method of identification of a marker for use in assessing susceptibility to at least one skin cancer selected from melanoma, basal cell carcinoma and squamous cell carcinoma, the method comprising:
a. identifying at least one polymorphic marker in linkage disequilibrium with at least one of the ASIP gene, the TYR gene and the TYRP1 gene;
b. determining the genotype status of a sample of individuals diagnosed with, or having a susceptibility to, the skin cancer; and
c. determining the genotype status of a sample of control individuals;
wherein a significant difference in frequency of at least one allele in at least one polymorphism in individuals diagnosed with the skin cancer, as compared with the frequency of the at least one allele in the control sample is indicative of the at least one polymorphism being useful for assessing susceptibility to the skin cancer.

42. The method according to claim 41, wherein an increase in frequency of the at least one allele in the at least one polymorphism in individuals diagnosed with, or having a susceptibility to, the skin cancer, as compared with the frequency of the at least one allele in the control sample is indicative of the at least one polymorphism being useful for assessing increased susceptibility to the skin cancer.

43. A method of genotyping a nucleic acid sample obtained from a human individual comprising determining the identity of at least one allele of at least one polymorphic marker in a nucleic acid sample from the individual, wherein the at least one marker is associated with at least one of the ASIP gene, the TYR gene and the TYRP1 gene, and wherein determination of the presence of the at least one allele in the sample is indicative of a susceptibility to at least one skin cancer selected from melanoma, basal cell carcinoma and squamous cell carcinoma in the individual.

44. The method according to claim 43; wherein genotyping comprises amplifying a segment of a nucleic acid that comprises the at least one polymorphic marker by Polymerase Chain Reaction (PCR), using a nucleotide primer pair flanking the at least one polymorphic marker.

45. The method according to any one of the claims 40-44, wherein the at least one marker associated with the TYR gene is a selected from the group consisting of rs1126809, and markers in linkage disequilibrium therewith.

46. The method according to claim 45, wherein the at least one marker in linkage disequilibrium with rs1126809 is selected from the group consisting of rs3913310, rs17184781, rs7120151, rs7126679, rs11018434, rs17791976, rs7931721, rs11018440, rs11018441, rs10830204, rs11018449, rs477424, rs7929744, rs7127487, rs10830206, rs4121738, rs11018463, rs11018464, rs3921012, rs7944714, rs10765186, rs9665831, rs1942497, rs2156123, rs7930256, rs4420272, rs7480884, rs12363323, rs1942486, rs10830216, rs17792911, rs4121729, rs10830219, rs10830228, rs10830231, rs7127661, rs10830236, rs949537, rs5021654, rs12270717, rs621313, rs7129973, rs11018525, rs17793678, rs594647, rs10765196, rs10765197, rs7123654, rs11018528, rs12791412, rs12789914, rs7107143, rs574028, rs2000553, rs11018541, rs10765198, rs7358418, rs10765200, rs10765201, rs4396293, rs2186640, rs10501698, rs10830250, rs7924589, rs4121401, rs10741305, rs591260, rs1847134, rs1393350, rs1126809, rs1827430, rs3900053, rs1847142, rs501301, rs4121403, rs10830253, rs7951935, rs1502259, rs1847140, rs1806319, rs4106039, rs4106040, rs10830256, rs3793973 and rs1847137.

47. The method according to any one of the claims 40-44, wherein the at least one marker associated with the TYRP1 gene is a selected from the group consisting of rs1408799, and markers in linkage disequilibrium therewith.

48. The method according to claim 47, wherein the at least one marker in linkage disequilibrium with rs1408799 is selected from the group consisting of rs791675, rs1325131, rs10756375, rs1590487, rs791691, rs791696, rs791697, rs702132, rs702133, rs702134, rs10960708, rs10809797, rs10429629, rs10960710, rs1022901, rs962298, rs6474717, rs1325112, rs1325113, rs4428755, rs10756380, rs10756384, rs13283146, rs1408790, rs1408791, rs10960716, rs713596, rs1325115, rs1325116, rs1408792, rs10809806, rs13288558, rs2025556, rs1325117, rs6474718, rs13283649, rs1325118, rs10738286, rs7466934, rs10960721, rs7036899, rs10756386, rs10960723, rs4612469, rs977888, rs10809808, rs10756387, rs10960730, rs10809809, rs10125059, rs10756388, rs10960731, rs10960732, rs7026116, rs10124166, rs7047297, rs13301970, rs10960735, rs1325122, rs6474720, rs6474721, rs10960738, rs13283345, rs10809811, rs1408794, rs1408795, rs13294940, rs1325124, rs996697, rs2382359, rs995263, rs1325125, rs10435754, rs4741242, rs2209275, rs7022317, rs1121541, rs10809818, rs1325127, rs10960748, rs9298679, rs9298680, rs7863161, rs1041105, rs10960749, rs1408799, rs1408800, rs13294134, rs16929340, rs13299830, rs10960751, rs10960752, rs10960753, rs16929342, rs16929345, rs16929346, rs13296454, rs13297008, rs10116013, rs10809826, rs7847593, rs13293905, rs2762460, rs2762461, rs2762462, rs2762463, rs2224863, rs2733830, rs2733831, rs2733832, rs2733833, rs2209277, rs2733834, rs683, rs2762464, rs910, rs1063380, rs9298681, rs10960758, rs10960759, rs12379024, rs13295868, rs7019226, rs11789751, rs10491744, rs10960760, rs2382361, rs1409626, rs1409630, rs13288475, rs13288636, rs13288681, rs1326798, rs7871257, rs12379260, rs13284453, rs13284898, rs7048117, rs10756400, rs970944, rs970945, rs970946, rs970947, rs10960774, rs10756402, rs10756403, rs10738290, rs13300005, rs10756406, rs7019486, rs927868, rs7019981, rs927869, rs4741245, rs7023927, rs7035500, rs13302551, rs1543587, rs1074789, rs2181816, rs10125771, rs10960779, rs1326789, rs7025842, rs7025953, rs7025771, rs7025914, rs10491743, rs1326790, rs1326791, rs1326792, rs7030485, rs10960781, rs12115198, rs10960783, rs1041176, rs10119113, rs1326795, rs2209273, rs7855624, rs10491742, and rs3750502.

49. The method according to any one of the claims 40-44, wherein the at least one polymorphic marker associated with the ASIP gene is selected from markers in linkage disequilibrium with the haplotype comprising rs1015362 allele A and rs4911414 allele T.

50. The method according to claim 49, wherein the at least one polymorphic marker is selected from the group consisting of rs1885120, rs17401449, rs291671, rs291695, rs293721, rs721970, rs910873, rs17305573, rs4911442, rs1204552, rs293709, rs6058091, rs1884431, rs6142199, rs2068474, rs2378199, rs2378249, rs2425003, rs4302281, rs4564863, rs4911430, rs6059928, rs6059937, rs6059961, rs6059969, rs6087607, rs2144956, rs2295443, rs2889849, rs6058089, rs6059916, rs932542, rs17421899, rs1884432, rs7265992, rs17092148, rs3787220, rs3787223, rs6058115, rs6060009, rs6060017, rs6060030, rs6060034, rs6060043, rs6060047, rs6088594, rs7271289, rs910871, rs6088316, rs17396317, rs2425067, rs6058339, rs6060612, rs2378412, rs293738, rs1205339, rs2281695, rs4911154, rs6088515, rs7269526, rs17305657, rs1122174, rs6060025, rs6059908, rs4911523, rs4911315, rs619865, rs6059931, rs11546155, rs221981, rs17122844, rs7272741, rs2425020, rs2424941, rs761930, rs221984, rs2378078, rs2424944, rs633784, rs666210, rs7361656, rs2424948, rs2424994, rs221985, rs17092378, rs2050652, rs6058192, rs6059662, and rs7274811.

51. The method according to any one of the claims 40-44, wherein the at least one marker associated with the ASIP gene is the haplotype comprising rs1015362 allele A and rs4911414 allele T.

52. A kit for assessing susceptibility to at least one skin cancer selected from melanoma, basal cell carcinoma and squamous cell carcinoma in a human individual, the kit comprising reagents for selectively detecting at least one allele of at least one polymorphic marker in the genome of the human individual, wherein the polymorphic marker is a marker associated with at least one of the ASIP gene, the TYR gene and the TYRP1 gene and a collection of data comprising correlation data between the at least one polymorphic marker and susceptibility to the skin cancer in humans.

53. The kit according to claim 52, wherein the at least one marker associated with the TYR gene is a selected from the group consisting of rs1126809, and markers in linkage disequilibrium therewith.

54. The kit according to claim 53, wherein the at least one marker in linkage disequilibrium with rs1126809 is selected from the group consisting of rs3913310, rs17184781, rs7120151, rs7126679, rs11018434, rs17791976, rs7931721, rs11018440, rs11018441, rs10830204, rs11018449, rs477424, rs7929744, rs7127487, rs10830206, rs4121738, rs11018463, rs11018464, rs3921012, rs7944714, rs10765186, rs9665831, rs1942497, rs2156123, rs7930256, rs4420272, rs7480884, rs12363323, rs1942486, rs10830216, rs17792911, rs4121729, rs10830219, rs10830228, rs10830231, rs7127661, rs10830236, rs949537, rs5021654, rs12270717, rs621313, rs7129973, rs11018525, rs17793678, rs594647, rs10765196, rs10765197, rs7123654, rs11018528, rs12791412, rs12789914, rs7107143, rs574028, rs2000553, rs11018541, rs10765198, rs7358418, rs10765200, rs10765201, rs4396293, rs2186640, rs10501698, rs10830250, rs7924589, rs4121401, rs10741305, rs591260, rs1847134, rs1393350, rs1126809, rs1827430, rs3900053, rs1847142, rs501301, rs4121403, rs10830253, rs7951935, rs1502259, rs1847140, rs1806319, rs4106039, rs4106040, rs10830256, rs3793973 and rs1847137.

55. The kit according to claim 52, wherein the at least one marker associated with the TYRP1 gene is a selected from the group consisting of rs1408799, and markers in linkage disequilibrium therewith.

56. The kit according to claim 52, wherein the at least one marker in linkage disequilibrium with rs1408799 is selected from the group consisting of rs791675, rs1325131, rs10756375, rs1590487, rs791691, rs791696, rs791697, rs702132, rs702133, rs702134, rs10960708, rs10809797, rs10429629, rs10960710, rs1022901, rs962298, rs6474717, rs1325112, rs1325113, rs4428755, rs10756380, rs10756384, rs13283146, rs1408790, rs1408791, rs10960716, rs713596, rs1325115, rs1325116, rs1408792, rs10809806, rs13288558, rs2025556, rs1325117, rs6474718, rs13283649, rs1325118, rs10738286, rs7466934, rs10960721, rs7036899, rs10756386, rs10960723, rs4612469, rs977888, rs10809808, rs10756387, rs10960730, rs10809809, rs10125059, rs10756388, rs10960731, rs10960732, rs7026116, rs10124166, rs7047297, rs13301970, rs10960735, rs1325122, rs6474720, rs6474721, rs10960738, rs13283345, rs10809811, rs1408794, rs1408795, rs13294940, rs1325124, rs996697, rs2382359, rs995263, rs1325125, rs10435754, rs4741242, rs2209275, rs7022317, rs1121541, rs10809818, rs1325127, rs10960748, rs9298679, rs9298680, rs7863161, rs1041105, rs10960749, rs1408799, rs1408800, rs13294134, rs16929340, rs13299830, rs10960751, rs10960752, rs10960753, rs16929342, rs16929345, rs16929346, rs13296454, rs13297008, rs10116013, rs10809826, rs7847593, rs13293905, rs2762460, rs2762461, rs2762462, rs2762463, rs2224863, rs2733830, rs2733831, rs2733832, rs2733833, rs2209277, rs2733834, rs683, rs2762464, rs910, rs1063380, rs9298681, rs10960758, rs10960759, rs12379024, rs13295868, rs7019226, rs11789751, rs10491744, rs10960760, rs2382361, rs1409626, rs1409630, rs13288475, rs13288636, rs13288681, rs1326798, rs7871257, rs12379260, rs13284453, rs13284898, rs7048117, rs10756400, rs970944, rs970945, rs970946, rs970947, rs10960774, rs10756402, rs10756403, rs10738290, rs13300005, rs10756406, rs7019486, rs927868, rs7019981, rs927869, rs4741245, rs7023927, rs7035500, rs13302551, rs1543587, rs1074789, rs2181816, rs10125771, rs10960779, rs1326789, rs7025842, rs7025953, rs7025771, rs7025914, rs10491743, rs1326790, rs1326791, rs1326792, rs7030485, rs10960781, rs12115198, rs10960783, rs1041176, rs10119113, rs1326795, rs2209273, rs7855624, rs10491742, and rs3750502.

57. The kit according to claim 52, wherein the at least one polymorphic marker associated with the ASIP gene is selected from markers in linkage disequilibrium with the haplotype comprising rs1015362 allele A and rs4911414 allele T.

58. The kit according to claim 57, wherein the at least one polymorphic marker is selected from the group consisting of rs1885120, rs17401449, rs291671, rs291695, rs293721, rs721970, rs910873, rs17305573, rs4911442, rs1204552, rs293709, rs6058091, rs1884431, rs6142199, rs2068474, rs2378199, rs2378249, rs2425003, rs4302281, rs4564863, rs4911430, rs6059928, rs6059937, rs6059961, rs6059969, rs6087607, rs2144956, rs2295443, rs2889849, rs6058089, rs6059916, rs932542, rs17421899, rs1884432, rs7265992, rs17092148, rs3787220, rs3787223, rs6058115, rs6060009, rs6060017, rs6060030, rs6060034, rs6060043, rs6060047, rs6088594, rs7271289, rs910871, rs6088316, rs17396317, rs2425067, rs6058339, rs6060612, rs2378412, rs293738, rs1205339, rs2281695, rs4911154, rs6088515, rs7269526, rs17305657, rs1122174, rs6060025, rs6059908, rs4911523, rs4911315, rs619865, rs6059931, rs11546155, rs221981, rs17122844, rs7272741, rs2425020, rs2424941, rs761930, rs221984, rs2378078, rs2424944, rs633784, rs666210, rs7361656, rs2424948, rs2424994, rs221985, rs17092378, rs2050652, rs6058192, rs6059662, and rs7274811.

59. The kit according to any claim 52, wherein the at least one marker associated with the ASIP gene is the haplotype comprising rs1015362 allele A and rs4911414 allele T.65.

60. The kit according to any one of claims 52-59, wherein the collection of data is on a computer-readable medium.

61. A computer-readable medium having computer executable instructions for determining susceptibility to a skin cancer selected from melanoma, basal cell carcinoma and squamous cell carcinoma, the computer readable medium comprising:
data indicative of at least one polymorphic marker;
a routine stored on the computer readable medium and adapted to be executed by a processor to determine risk of developing the at least skin cancer for the at least one polymorphic marker;
wherein the at least one polymorphic marker is selected from the group consisting of rs1015362, rs4911414, rs1126809, rs1408799, rs6060043, and rs1393350, and markers in linkage disequilibrium therewith.

62. The computer readable medium of claim 61, wherein the computer readable medium contains data indicative of at least two polymorphic markers.

63. The computer readable medium of claim 61 or claim 62, wherein the at least one polymorphic marker is selected from the markers set forth in Tables 14, 25 and 26.

64. An apparatus for determining a genetic indicator for a skin cancer selected from melanoma, basal cell carcinoma and squamous cell carcinoma in a human individual, comprising:
a processor
a computer readable memory having computer executable instructions adapted to be executed on the processor to analyze marker and/or haplotype information for at least one human individual with respect to at least one polymorphic marker or a haplotype comprising two or more markers selected from the group consisting of rs1015362, rs4911414, rs1126809, rs1408799, rs6060043, and rs1393350, and markers in linkage disequilibrium therewith, and generate an output based on the marker or haplotype information, wherein the output comprises a measure of susceptibility of the at least one marker or haplotype as a genetic indicator of the skin cancer for the human individual.

65. The apparatus according to claim 64, wherein the computer readable memory further comprises data indicative of the frequency of at least one allele of at least one polymorphic marker or the at least one haplotype in a plurality of individuals diagnosed with, or presenting symptoms associated with, the at least one skin cancer, and data indicative of the frequency of at the least one allele of at least one polymorphic marker or the at least one haplotype in a plurality of reference individuals, and wherein a measure of susceptibility is based on a comparison of the at least one marker and/or haplotype status for the human individual to the data indicative of the frequency of the at least one marker and/or haplotype information for the plurality of individuals diagnosed with the skin cancer.

66. The apparatus according to claim 65, wherein the computer readable memory further comprises data indicative of the risk of developing at least one skin cancer associated with at least one allele of the at least one polymorphic marker or the at least one haplotype, and wherein a measure of susceptibility for the human individual is based on a comparison of the at least one marker and/or haplotype status for the human individual to the risk associated with the at least one allele of the at least one polymorphic marker or the at least one haplotype.

67. The apparatus according to claim 66, wherein the computer readable memory further comprises data indicative of the frequency of at least one allele of at least one polymorphic marker or at least one haplotype in a plurality of individuals diagnosed with, or presenting symptoms associated with, the at least one skin cancer, and data indicative of the frequency of at the least one allele of at least one polymorphic marker or at least one haplotype in a plurality of reference individuals, and wherein risk of developing the at least one skin cancer is based on a comparison of the frequency of the at least one allele or haplotype in individuals diagnosed with, or presenting symptoms associated with, the skin cancer, and reference individuals.

68. The apparatus according to any one of claims 64-67, wherein the at least one polymorphic marker is selected from the markers set forth in Tables 14, 25 and 26.

69. A method of inferring at least one pigmentation trait of a human individual, the method comprising determining the identity of at least one allele of at least one polymorphic marker in a nucleic acid sample from the individual, or in a genotype dataset from the individual wherein the at least one marker is selected from the group of markers set forth in Table 10, and markers in linkage disequilibrium therewith, and wherein determination of the presence or absence of the at least one allele is indicative of the at least one pigmentation trait of the individual.

70. The method of claim 69, wherein the at least one polymorphic marker is selected from the markers set forth in Table 10B-10D, and markers in linkage disequilibrium therewith.

71. The method of claim 69, wherein the at least one polymorphic marker is selected from the markers set forth in Table 10C-10D, and markers in linkage disequilibrium therewith.

72. The method according to claims 70 or claim 71, further comprising determining the identity of at least one allele of at least one polymorphic marker selected from the markers set forth in Table 10A.

73. The method according to claim 71 or 72, further comprising further comprising determining the identity of at least one allele of at least one polymorphic marker selected from the markers set forth in Table 10B.

74. The method of claim 69, comprising determining the identity of at least one allele of each of the polymorphic markers rs12896399, rs12821256, rs1540771, rs1393350, rs1042602, rs1667394, rs7495174, rs1805008, rs1805007, or markers in linkage disequilibrium therewith.

75. The method of claim 74, further comprising determining the identity of at least one allele of at least one marker selected from the markers set forth in Table 10D, and markers in linkage disequilibrium therewith.

76. The method according to any of the claims 69-75, wherein the pigmentation trait is selected from skin pigmentation, eye pigmentation and hair pigmentation.

77. The method according to claim 76, wherein the pigmentation trait is hair pigmentation and the at least one polymorphic marker is selected from rs896978, rs3750965, rs2305498, rs1011176, rs4842602, rs995030, rs1022034, rs3782181, rs12821256, rs4904864, rs4904868, rs2402130, rs7495174, rs7183877, rs8039195, rs1667394 and rs1540771, and markers in linkage disequilibrium therewith.

78. The method according to claim 77, wherein the at least one polymorphic marker is selected from rs896978, rs3750965, rs2305498, rs1011176, rs4842602, rs995030, rs1022034, rs3782181, rs12821256, rs4904864, rs4904868, rs2402130 and rs1540771, and markers in linkage disequilibrium therewith.

79. The method according to claim 76, wherein the pigmentation trait is eye colour, and wherein the at least one polymorphic marker is selected from rs1022901, rs10809808, rs11206611, rs12441723, rs1393350, rs1408799, rs1448488, rs1498519, rs1584407, rs1667394, rs16950979, rs16950987, rs1907001, rs2240204, rs2402130, rs2594935, rs2703952, rs2871875, rs4453582, rs4778220, rs4904864, rs4904868, rs630446, rs6497238, rs7165740, rs7170869, rs7183877, rs728405, rs7495174, rs7680366, rs7684457, rs8016079, rs8028689, rs8039195, rs927869, and markers in linkage disequilibrium therewith.

80. The method according to claim 79, wherein the at least one polymorphic marker is selected from rs4453582, rs7684457, rs7680366, rs11206611, rs1393350, rs8016079, rs4904864, rs4904868, rs2402130, rs1408799, rs630446, rs11206611, rs1393350, rs1022901, rs10809808 and rs927869, and markers in linkage disequilibrium therewith.

81. The method according to claim 76, wherein the pigmentation trait is skin pigmentation, and the at least one polymorphic marker is selected from rs4911379, rs2284378, rs4911414, rs2225837, rs6120650, rs2281695, rs6059909, rs2378199, rs2378249, rs6060034, rs6060043, rs619865, rs11242867, rs9378805, rs9328192, rs9405681, rs4959270, rs1540771, rs1393350, rs1042602, rs1050975, rs872071, rs7757906, rs950286, rs9328192, rs9405675 and rs950039, and markers in linkage disequilibrium therewith.

82. The method according to claim 81, wherein the at least one polymorphic marker is selected from rs1042602, rs1050975, rs11242867, rs1393350, rs1540771, rs2225837, rs2281695, rs2284378, rs2378199, rs2378249, rs4911379, rs4911414, rs4959270, rs6059909, rs6060034, rs6060043, rs6120650, rs619865, rs7757906, rs872071, rs9328192, rs9378805, rs9405675, rs9405681, rs950039 and rs950286, and markers in linkage disequilibrium therewith.

83. A kit for assessing the natural pigmentation pattern of a human individual, the kit comprising reagents for selectively detecting at least one allele of at least one polymorphic marker in the genome of the human individual, wherein the polymorphic marker is selected from the markers set forth in Table 10, and markers in linkage disequilibrium therewith, and a collection of data comprising correlation data between the at least one polymorphic marker and the natural pigmentation pattern in humans.

84. The kit according to claim 83, wherein the pigmentation pattern comprises at least one of hair colour, eye colour, skin colour and skin sensitivity to sunlight.

85. The kit according to claim 83 or claim 84, wherein the at least one polymorphic marker is characterized by the sequence as set forth in SEQ ID NO: 1-134)

86. The kit according to any one of claims 83-85, wherein the reagents comprise at least one contiguous oligonucleotide that hybridizes to a fragment of the genome of the individual comprising the at least one polymorphic marker, a buffer and a detectable label.

87. The method, kit, use, medium or apparatus according to any of the preceding claims, wherein linkage disequilibrium between markers is characterized by particular numerical values of the linkage disequilibrium measures r² and/or |D′|.

88. The method, kit, use, medium or apparatus according to any of the preceding claims, wherein linkage disequilibrium between markers is characterized by values of r² of at least 0.1.

89. The method, kit, use, medium or apparatus according to any of the preceding claims, wherein linkage disequilibrium between markers is characterized by values of r² of at least 0.2.


SEQUENCE LISTING (483 sequences)

SEQ ID NO: 1 - DNA, 599 bp, Homo sapiens
ttcacttcttttatttttagaacaagtgactcatctccaccacaggggctctgtgagctgctcccggacaggcttacagaggccaagcagggagtgagaggctcgagagggctgcagctgcccctgcccacactctgaggcccatgagtggctgagatgcaaggcagtcggtcgttcccactgacctgcccctccaaaatagcaagtctggagaagccgggcacctgcagaagaggtggctgcggcagagggtgcatccatctcgtcccgcctaggaggggagaggtggatgagatcagrgagttgtttctcagggtctcacctcaccatcttttcctccacatgcagaccggccatggccatggccacagccacgggctgcccaggtgtgttgcacctgcctggctgcatcctttctcacatacctgcctccacagaggaaaagctgggagcagtctggggacccagaagagccacccctttcccggggctgtggccccgttggtgggggaatggggaggaggaattcctagtgatgagatccgtgcccaggaccgagggcccacagctcacctttgcctggcctcagtccacctcac

SEQ ID NO: 2 - DNA, 599 bp, Homo sapiens
gcaacaggccccatgaaaagactggaaccaggattggaaactatcagtttttccttctccatctctgaatctctgtttctccgcctcattctatttgtgcagatattttctcctttgttccagtcacttggtagaatgtaactgcccaagagctgccaggtttacatatttcaatttcagccctattcagagattaattatcagtttctaatgatcaataccaattttggggaggaacgttctggttggcctatccaagtcaggtgtctaccatcatccaaccaactatggctacagggmaagttcaccgtttgtaagcatggattctgaggctccactcctatgatgcggcattctctctataaaaatagacgtcagccaaatagacacttgtagtcaactatttatctgcctcgatctcccatgtaataagaaagtatttatttaaaaggaataagcataaaatgttagttgaatgaataaaatatgacattaaatgctattttaagtagttcttttgtatactgtactacagcacaggggcccgtcttcctaataagtgattatattaagatgctgttcagattatctaggccatt

SEQ ID NO: 3 - DNA, 599 bp, Homo sapiens
ggagcttgcagtgagcggagatcacgccactgcactccagcctgggctacagagcgagactccgtctcataaaataaaatagatagatagatagatagatagatagatagatagatagatagatagatagatagataaaggataatgtactcacataactaaaaacctcagagaacaggctggcttcagatgtgatctgtgacaggacatcaacagatttccccaggacttggttcctctatctcttatctttgtgaacttttagatggctttcttctcagataagtagcaagatgtttrcatcttgagaggtaaaatttacctctaaattttacatcttgtgttgttcaaatattttctacttaagcaaagccctatggcaaatacttttaaattattttttatcttttagagacagagtctggctttgctgcccaggctggagtgcagtggtgcaatcatagctcattgctgcttcagactcctgggctcaaggaaccctcccgcgtcagcctccccaatacctgggactacaggtgcacatcaccatgctcagctaatttttaaacttcttgtagagacggagtcttgctatcttc

SEQ ID NO: 4 - DNA, 599 bp, Homo sapiens
tggcaacttcatgggattcaactgtggaaactgcaagtttggcttttggggaccaaactgcacagagagacgactcttggtgagaagaaacatcttcgatttgagtgccccagagaaggacaaattttttgcctacctcactttagcaaagcataccatcagctcagactatgtcatccccatagggacctatggccaaatgaaaaatggatcaacacccatgtttaacgacatcaatatttatgacctctttgtctggatgcattattatgtgtcaatggatgcactgcttgggggatmtgaaatctggagagacattgattttgcccatgaagcaccagcttttctgccttggcatagactcttcttgttgcggtgggaacaagaaatccagaagctgacaggagatgaaaacttcactattccatattgggactggcgggatgcagaaaagtgtgacatttgcacagatgagtacatgggaggtcagcaccccacaaatcctaacttactcagcccagcatcattcttctcctcttggcaggtaagatatgctagatatacgatgtcagagtagggaggaaccttaacaatcactt

SEQ ID NO: 5 - DNA, 599 bp, Homo sapiens
aagaggggatgaatcacccttggcccagcctccccacaaagcctgaccctgggcaggtgagtgacgggtgtgtcctcgtagagtctattgctgcctggacacctttcttttgggagctcaaagcaagtgagctcacctacctgccaccgcccaggaccagtctgcccactgcctaaatgatgcccggccagcaggacctggcctgcagatcccagtgagtcatgagcctcagccccctccagcccactggggctctcacctccacatgtgggtagaagctttcctgccccctcttcctcyagtagccctcagtgtcgaaggtgagcttgtaggtgcctgccttcatctggtccaggacagtgaccatctgggtctgtgtagctggggagaggatgaggctgcagagatggggaccagaagccccccaccccagctttcctgggtctgcatcccagtgggcctcagacactgccctgccacctgtcagacttgggtgagcagacacagtgaggctgttaggtcctgcagttccagagcagtctagggacaccactgccctgtctttaggaaatcacaacacagagaagcaaaaagggaaa

SEQ ID NO: 6 - DNA, 599 bp, Homo sapiens
actgtgacctccgcctcctgggttcaagagactctcctgcctcagcctccctggtagctgggattacaggtgtgagccactgcacccacccaagacaagtgattttcattgtaaatatttgactttagtgaaagcgtccaattgactgccctcttactgttttgaggaattcagaagtggagatttcagttcagcggttgaggagaattgcggcgagacaagcatggaaaatcagtgacatctgattggcagatgagcttatttcaaaaggaagggtggctttgcatttcttgtgttctrtagactgccatcattgatgatcactgtgaaaattgaccaagtgatgtgtttacatttactgaaatgcgctctttaatttgttgtagattaggtcttgctggaagacagagaaaacttgcctttcagtattgacactgactagagtgatgactgcttgtaggtatgtctgtgccatttctcagggaagtaagatgtaaattgaagaagcctcacacgtaaaagaaatgtattaatgtatgtaggagctgcagttcttgtggaagacacttgctgagtgaaggaaatgaatctttgactga

SEQ ID NO: 7 - DNA, 599 bp, Homo sapiens
gcctggccaacatggtgaaaccccatttctattaaaaatacaaaaattagccaggcatggtggcgcatgcctgtagtcccggctactcaggatgctgaggcaggagaatcgcttgaactcaggaggcggaggttgcagtgagccgagatggtgccactatactccagcctggatgacggagccagaaaggtaggctgtaagacccctttgtttagacttccttgtactttgtaaggagctcatctctccgacctgactgttacatatttgcaatgtgactgcccatgtgccatgcctcakgcttatagtcctgcctatggaccacatcatgcaatggcaccgtgctggtacaggaagaagttatatgtaggaaccttgataaaagagcctggtggctattaaatggattactaaaagttgtttttaaatatgtttttcatgttgaactatgagggtgatatgaatggacgtataacaaaattggtaaactctcagaaattctcagggccctaaagaagatcaccctacatcaccaccatctcaagcatatcttccgcgagacacagaagaaacttgagaagccacaatgagcacatata

SEQ ID NO: 8 - DNA, 599 bp, Homo sapiens
ctgggccctctccagcttcaaatggaaaaacacaggcctgcgtggggcccgtcctgaggttctggactgggtgtgtcagtcagtgacgtcatcagggaccctggcccgctccctctcatgccttgtcgtggcctctgccaggcccgctgtcatccggcctgtcgtggcctctgcccggcctgctgtcatccggcttgtcgtggcctctgcccggcctgctgttgtccgacctgttgcctcatggttcccagatgggtgctgcagctccaagagtcacagcttctgaccactgcgttcagrgaaggaagagaaagagccagggaggaaaatctcttccagaaacctcatgcagttctcccctttcagctgactggctaagccgggtctcacagccgtgtctggctgccagggagctgggaaagttctgtggggtccctgcctttcggggggataaaggcaagggctcagagggaacagagttagccgtggccatggaaggaagagcagagcttcctccaggctgagtggggccggaacactgctgggcccagctctagtcggcgggaggctgtgtgctgtgttggggagcggatttggca

SEQ ID NO: 9 - DNA, 599 bp, Homo sapiens
tgtgtgatcatcaaggcaacttctggttggcaggttctgacccactttcctcactcttcctatttccttagcacctggtgtactaattgccagccccattgcctggagatatgctctagactgggtttcagaactctccttcagggcacacattttgactttgtctcctcgtaactggagaaaaattattctccttggtcaacctcagcctaaggctttataagtctcatggagacttgcttcagttttgttcagaaaaccagcatttactgagcctttgctctgtgttctcatgatcaygggtgctgaagacacagagtgggtcaaaagtagtaagtactctttaaattctttccatcttgaggggtgagggctcccaatctgttgttattgtctatttttctgtctagatgttgtaatagactgtgggtgaaggaagacttcatatgaggaaacagttcataatttgggatagggtgcttgtgttagtttcctaggactgccccaacaaaatatcacaaactggttggcttaaaacaccagaaatttattttctcataaattttttttttttttttgagacagtaccttgacctgtc

SEQ ID NO: 10 - DNA, 599 bp, Homo sapiens
aaatgaggaacttattgggaactggagtaatggtcgctttcgttacgcattagcaaagagcctctactctaaagatttgtggaactttgaacttgggagcaatgatttagggtatttggcagaagaaacttctaagcagtgaagtgtttaagatgtgacctggctgcttctaacagcttatgcttatatgtgtgaacaaagaaataacctgaaactggaacttatatttaaaatggaagcagagcataaaagtttgaaaaatttgcagcctggtcatgtgataaagaaaggaaaatttcrctagcacctctatctagggtctcaggttctcatctgacaaagtacacaaaggccttacaaccccatgtgtccatagatagaagaataaaaaaagacagtctgtaagagaggaaatcaaaagctgttcatggtagtagaaaggattaccaatgggtaccacaaaaagtcaagactcatataaataattaaaaacaaggcatttacactaatttgtataaatgtttctctcccaagctaaggtatttactaaggaaatcaatttggtggtgggtctaaagaactttatttaactctgtgtc

SEQ ID NO: 11 - DNA, 599 bp, Homo sapiens
gggtactgcacagagctgcgacctgtaggtaaatacctgtgcactgattgagtcaggtgtctgaagcccccagccagtgactgatgggctcagaaactgaacctgctttctctgccttttaagctgctgggaagtcctccttttttcccaaaggaggagggaaaacatattaacctccactcccaaatgccttgggctcccacagccctgcaggggaagcagaactgtctccactgacagaaatggaaattgaggctcacaggggcctgactgcccacagttcacagctggaaacagctyggagacccccaagccctcactcccaacctccaggcttcctatgtccacaaagctgctactgggaatacccaggagcctgggactatcagtgttaagaaagagctctggtccctcccacaaatgacaaatcaaaacccaaacccaccagccctggggcatttgtccctataccttggactgcagatacacagggtgggtggacctgctcctcccggggacagcccttcgggctccagaggatgccactgtgtgcggctgggggactgagaggttcccatgtagttccagagcggcaggct

SEQ ID NO: 12 - DNA, 599 bp, Homo sapiens
tcacgccattctcctgcctcagcctcctgagtagctgggactacaggtgcctgccaccacacccggctaattttttctattttttagtagagacggggtttcaccgtgttagccaggatgggctcgatctcctgacctcatgatccacctgcctcggcctcccaaagtgctgggattacaggtgtgagccactgggcccggcccctaactcgcactcttcacagtccgtgcaggaaggctttcctaactctccaatctgatatgcatgtccaacatcttcttcgcaaatctgtgagcacmtgaatcatcgcacttcttgtggatttagtggcttgtttcccaagctacgtgataatgagggcagggaccaaagcaataactgaaaaatctaatgaattctataatgtatgctggataaaagaatgggaaaggccggacgcagtggctcacacctgtaatcccagcacttgggaggccaagatgggcagatcacctgaggtcaggaattgaagaccagcctggccaacatggtgaaacctcatctctactaaagatacaaaaattgggcagggcgcggtgactcacacctgtaatcccgg

SEQ ID NO: 13 - DNA, 599 bp, Homo sapiens
ggtcccagacttaatccctgaatagctcgtgcttgaagccaactcacagagcacagcaaagggttttgaaaccgcactgacgttaactccatcccctaacgaaggggaaccagagcttgtgttctcaacctgagtggatttatcgtctgatgaagcagacacatgaaaatcaacattctctacattctcaacattctctctcattcaacatttttaatcagggttaaaatgtacatattaaagcgatctgggatgcaatctacaattatttgggatacaaagaagcaaaaaaatctgacyaattcttcagtcaatcaattaaaaatattttttaaatgacccagttctcaagggaaaaggcaattaacaaatgtcaaccctgagatgacatagatgttgaaattatcaaagactttaaagtgactgttataaccatgttccatgaggtaaaagatggacagttctgaaattaacagaaacatagaaattcgcagcagggcaatatgaaaaaaaaaagaaaaaaataggaattttagaatgaagtaaaaatattactggatgagttcaaaagcaaatgtatatgacagaagaaagaatct

SEQ ID NO: 14 - DNA, 599 bp, Homo sapiens
gtagagacggggtttcaccatgttggccaggatggtctcgatctcctgacctcgtgatctccccacctcagcgtcccaaagtgctggaattacaggcatgagccaccgcacccaaccatgaccccctctttttaacgtacagggtgtcctctgtccccagacccctgagcccaggttgaggagccaccagctgaggacggagccaaggtcacggagctgccgggtgagcgctgagcccaggtcagggagttgggtctcaggccgtcactgggactccctctgccttcaagaagcctcttycacccttggcccacagaggagacctggatggcactggggtccaggtgcagaagcctccagtgtccacgggcacctgagcttccagcaccttccctcctgctgggcggtggcctgtgcctggccgcaatggaggccgccttccccactgcccctctgagcctcagtctgtcctccctgcagccacctcccctggccaggtgccccggggtaactggtggaggctttcacgtaacttggtttccagctgcctctgtacacacacaggcacctgttccaggcttggcctctctgtacacaca

SEQ ID NO: 15 - DNA, 599 bp, Homo sapiens
tgttattagagcaaaataatggtataaaacagtctctttcttgaatttagtagggtgcttgacctgtcaggctgcaaagagagaagcttggttactgccattttgtgatggtcagccaaatggttttacaaagaagaggaaactgtgggtagatgaaagttctgcctataaagtaggtgttactacagtgcattgtggttatattaagtatgcccaaaggataaggaattcaggtgtgaagttgtgtggcagaagttgaaaattaattaagctctgtgtttagggtttttttcctttagygtgccgtagtaacatgcccttggctccagggaactttatgactaagttggaacaaagcactcatgactcatgttaaatgtagcagaggagagatcattaaatgtttcttggcaattaattacctgggatatgcctcttttttcatagctttggtttcaggtgtgtgagagagttgaaaacgtttaattgcacctaatttgttgctgtagttttatcatatgcctaagcattgtgataatgcctgtaacccgtgtgaagaattcgtaaggggcttcatttgtgacacccccagaggaggg

SEQ ID NO: 16 - DNA, 599 bp, Homo sapiens
cccactgaaatgtctcaaaaacatcttacagcacatctcatctagaatcatgacatcagcatcctcccctaatattccacttcttcaggggtttctgcatctggtaatacaaacactttgctcagtagcataagccagaaaagtaggcattttctgtggtacctggcccctcagttgtactgcttggatttccagacatcatctattttcctggacatcattttctcactgatttctttatccccctgatgcgtgcatatccaccaactcctactcttcctcagtcccttctctgcaacraaatctgtgtggtcttttacaaatgataatctgttcgtgttatcattcaccttcccagatacaaacagtgactttctgttaggaaaaaaacacaaatctccctaacatggcctctgctacccttcatgatttggcctcagttcaccttcctagcctcatctggagctatttgctccttgatccctatagacatattggtcaatttttaattccttgaatttgtcgcatacttccaatcaaagattctttccaaattccattctgtccttcagaaatattcttctaccatttctctaaac

SEQ ID NO: 17 - DNA, 599 bp, Homo sapiens
atgaaaggaataaaaccactcatcatagagaagcctatttattaattgtaaactgttgcaaggtcaagttttaagaatcattcaaaatcacacaaaattaaatttgaaagaataaacacacgacatttgaaaaaaggatgagaataaaaaccagtgttaggttatttctaacttgatctaagttgaacataatattgaattcattataagatatttgtaaggtattctggcctaaaaaaatttattatgaaatacagtaatcaaaatcaaaactggttcatccacttaatgaatgaataytgaccatgtgctccgagccagacaatgtgctaagttgttgagagagaaaagtgaacaagagagacatggtcctgtcctcatagcacttacagtatttccttttgaacagtgttgtatttattatatttagctcaatataaatctaaaattttgaacgattatatttatactttcctggcactaaacatgcatgtgtattgcaggaaaatgtataaaatagaaacatggtttaggaaactgtaaaactatcacttcaatacttaatacttttatttttctgtacaccttattacttaaat

SEQ ID NO: 18 - DNA, 599 bp, Homo sapiens
taacgagaacattggagactaggaagaggggaggtcaggaggggttgatggatgaaaaactacatattatgtacaatgtccactactctggtgatgggtgcactaaaatcccagacttcaccagtatataatttatccatgtaaccgaaaaccacttgcacccctaaagctaatgaaaaaaaaattcacaaagaaaatgaatacttatgtagtttggaggaataataaacaaatcagcactagagtttggaacagtaaagaagtcacatctgaaaactcgtccacattttccgagcattrggcagaaacgacaggaagttgtgaggaaagttcttgccacctaccattgctgcacgaggcttaggtctctcaggtctcttgggataactctctgtcggtggaatttccagcactggacaggtaggtagggaggacctccaggacccaccacatggacaaccacttattccccagccacaagcctcccttgggtaagcatgagtcggcctggctgtagcatccacgcgcaccaacccactgcaccttcagctcctaacaagctgacaggataaagttttactttgctggttacatttgct

SEQ ID NO: 19 - DNA, 599 bp, Homo sapiens
gctcctggatcctgcacaacgtccctgacatgtcggccagcagcacagacccagcctcaccctcagggcaagctcagcagaccccgtcatcctcaccaggggggtgctgggtagctgagccaggtcagggccagcacggcgaggggcctcaggcggcctgggcccacagagccaacgcgtgtggagggcccctggctgtacctcacgccacctgcgagtttcagtgcccaggaatccccagaaggaaatggccattggtgacggcaagcctgttctgggcataacacaacacactcaagygagacggctgcatcaaccacacagtccgctaggcacagtgtgagtggagtcgcgtggtctgggaggagactccagggaagagccagaacgccacacgctggggctggatggaccctggggggcctgagaacaggagggcaggggaggccgtgcacagctcaggccgtcaccgaggacagacgggtgggctctgcctcccccatgaggaatccctgtgcctgctgggaaagaccccagtgggtaggaggaggtgggaacccagtcaggccccgcggcccagggcagagagtgcacagggc

SEQ ID NO: 20 - DNA, 599 bp, Homo sapiens
tagcctgcctgtgttcaagggatggctgggaggtaggtgccctcctgggaagcagggctgctgggctgggaggagactgtgcctcagggaaaaggcgaggctgccttggtactgggtaggagtgagtggtgaggacctagccaaaggtaggtgagaggacaccctctgtgtccatttaacaaagcagagtttcagacagggtgtgaggcttccagagagacatttaacattcactgagcttcaggagagctgaccaccccaagccaaggccaacactgcctagtgtcctcactaagcaamccctcaggacacaggttccaactcagccactgagggagagagagacaaggcggtgtcagtgtccttcatagagaaggagcgaatgtctgggttggctgctcccagattccttagctgggaactctgagcacacattcttttcagagatgaccatagggtccttctcagggcatgcttcagtgccatgagtagaacatccatactggatttttttatttaaaaaatgtcaactattccatcaaaatgttatgttgactatatggtcaaataatattttggatatgttaggttaaataaaa

SEQ ID NO: 21 - DNA, 599 bp, Homo sapiens
ggtgttcctggtcaggttgttcctggaaggctcccctttcaacagcagctcccgcctcccggggctccaggatgagctggcctgtggcgcgctctccacccagtcctcagctggaaatgggaaacctgaggcttgcacggcaggacttctctggggagggccaggccaggacacggtcctcctgcccacattccttcagcctggctcagccccatgcccccacactgaagaccacactcaagtccagggcacatggagtctggccatgtgcccaccacacacgtgatagactgagctcarccaactcgtgcagttcataacacagaaaccctcctctctcttctaccataaactgccaagcttgcctcatttccttccttcagaggatacgagacgtagatgtccacaggttactgtcatgttttctaccctagtccttgcctttcgaatgtctttgaagactcaccaagcaatataagatcaaaattcctgggtctcatcaataccagatcaccagccccggttttcagtccccaatcctaaaccgggctccgggagctggatgttccatctgtgccccgaggcgtccctgctctgac

SEQ ID NO: 22 - DNA, 599 bp, Homo sapiens
atccatgcacagacatgtgcatacatgcctaacacatccacacggacacgtgcttagccgggacctgcaggctccaggctgagtcctaggaattgagatgctagttctgcagttcctgtaggtccacctgtcaggcatcttcctccccggggtgtgacatccggctctgaaacccagaggggcccctagggcagggcagtcaggggtcctgccggccggtgtggagacccccaggtgagaccaatgggttccctgagtcttcagcccctcctgacctcgaattagagcctgacccttgcygagaatagcctgggtatgacctgcctggatgtggacttatttcccaaataagaagcagaaagtgtgtagctgccaggaccccacagcaagggccaccatgggacccagtccagggactgcctgcagcaggcggaggagctcctcctcgggggtggatttgagatttggaaacagcctcagtcaagctggacaccagctcccgctgtgtgaccctggacaagccatcacagtctcagtgagtcagagccttgttccttgcctgtcacgcggatatagcagctttgaaacatggctctaga

SEQ ID NO: 23 - DNA, 599 bp, Homo sapiens
cattttctcacatcataaaatgttcctgaatcattttgcattttgcctgctcttgccctggaatcaactgcctctcaaaagatttctcattccttgtattggggaatggtgtttagaaatcaaaatctggatgcttggtgtgtgtgtagcaatttagatgtcgctgaatctaggcctccgcaatcagcatagttgggcatatgtgtttgtataccaacacccatatccacacatccatatctgtatctatatattaatttactcgtctatgtgtttaaaaccttcagatcatattgattmcctgaattccaatccagcaccataagatttgcctcttgctctcactgtctacaatatatactcacttatttgttcaattcctagcatacacctaaagtagttacagaattgctaactcatatatttgtgagaaatagatttactaactcaactacagtatttgtgtagacttctttttgtctttagtcttacagtagcctgtcaaaatatggttttctgaaatttcttaaattaattcttttcttctccatagccttcagtatgtttatgttatttatttgtaatgcacttattggggc

SEQ ID NO: 24 - DNA, 599 bp, Homo sapiens
ccgcatcactcagctccaccattcagtgcccagtgcaacatgaaaatgcggcacccttattcacacatcgcaagaatctcagggcctctcagggcctcacagtggcgggaggacggggggcaggtagagggaggcacatgtcttccgagcaccagaccggggccgaaagccacgcgtggactgcattttgcaccgtttctgaggctcaaagccacggacttaattcaacacgagggcgtcctgggggttgggcagggccccgtgtcctggtaggctgtgggttcccctctcacgaccacygttcaagggcagcgtgggtgagctggttcaatttctcggcctcagtctcttcgtctgtcaagtagaagtcatggatgtacctactttacgacagtcctgctgtgagcacaaaggtaccagccactcagatcaccctcggatcaaggtagtcaccttacacctgtcttttccgtggggttttatagtttgtcccagagtttccagttgcctttcttctcctgccctacactgggtgcccttatcttccatcttccaagctctggactctgcaacctccctccctgcccccagagcctccc

SEQ ID NO: 25 - DNA, 599 bp, Homo sapiens
gtgattttaatttcaaaaaatttgttaggtttgttttatgcctcagaatatgttctaacttagtatttgttttgtggatgcttgaacagattatgtattctgctgttattggctggagtgttctgtaaattttgattggatccagttgattgatggtgatgttgaattctatatcttggcagcttttctgtcttctaattttatcagctgtagagagagactttgaggtctccaactataaaagtataaatgtctttttctcctttcagttctattcattgtttctttgtttgtttggtrtatgcacgttttgaattgctgcgtcttaatggtggattgaccaagttctcattttgtaatgttgccatctgttcctggtaattatcttttttttttttttttttttttttttttttccgagacggagtttcgctccttttgcccaggcaggagtgaagtggcacgatctcagctcactgcaacctccaaccccaccgggttcaagtgattctcctgcctcagcctcctgagtagctgggattataggcatctgccatcacacctagctactttttgtatttttagtagagatggggttt

SEQ ID NO: 26 - DNA, 599 bp, Homo sapiens
tacataaaatcaatactatgaaagggtgtgtaccaaaatccgaccaatggttttcttcaagtgacaggatcacaagtggatttttcgttttgttgtttaacaactttcatcaaatgtcttttgtaaaggaaaagttttctctttaagaatcaaagaaatgtataaggaattttaaatgtcttgggaaaatgctcaagaaatgacctcccaaagtgagattctactgggaggaagcagacgataaggagataaatacataaacggagggagaataaaccccacatgggagaccaggccggrgaagagctggaagaagagcagcagagccgcagagggtagcctggctcgccccagctccggcgtcctccactcgaggtctgctcctggccgcgggtgaagccctgcgtgcttgacccaccctatggggcattgccctcttctgacggtggtgctcatcaccctcctgtgtctatttaagcttttcactacttgtgtatgttaatttcacaagtcacagtttcattctgcatgttctaaaaggttatgcatatggtacaccataaaaatcatcctgtacaagagagaaaaggctagaatac

SEQ ID NO: 27 - DNA, 599 bp, Homo sapiens
gcctggaattccagctactcaggaggctgaggcacgagaattgcctgaacccaggaggaggaggttgcagtgagctgagattttgtcactgcactccagcctgggtgacagagtgagactctgtctcccaaaaaaaaaagttactaacacagaactttgaagtcaatattagtcaatatttatcagcagcacatctcctaaacagagaaaaacactagccattattttcatgttacaaaaccaaattcaatataaaaacgatgtcttgataaaattacccagcatgcatgactcatgaarcacatttatggacatattttagaggaagataaagtaacaagttagaatacacattctaactagtgagtaagtgatatttcattatattattctctcttcttctatgtatctttgaaaatttccatactaacaagtgttttgtaagtagctagaaggcaatcacaggactgaaaataattcaaaggattctggtctttcaattaagcatgtgtctgcctcagagcacagatcattataagggaattgtaagttacctcccaccaatcccctctaacagcacagctcccagtggcgaggtg

SEQ ID NO: 28 - DNA, 599 bp, Homo sapiens
gcaacactgcactctagcctaggcaacagagtgagactgtctcaaaaaaaaaaacccaaaaaaacaaaaaaacaaaaaaaaaacccacatgaaattaaaaaaaaaaaaaaaaaagcaaacatgagatcctaaaaccctcactgcagtacaactcgctcttcctgcgtaactgggtgctgagaacgtctctccacgtcaccactcagacctgctcactgttttgagggagactgaacagtgacataatatatctacccacctccttggcctaggtgagaaaagggtttcttagcagttgcyttgactggcacacacgcactcactgtgtgtccacgtggacacctccagttgcaatgtttgtgttgtttcaccataaacggcttggggaaggggaaggggaaggggaagggcctggctgagaggctcagagcagatctgcaggaggctcttggggaggccaaggcagtcctcagatgcagcaggtgagcgaagcaccagaaatcatggatgtggcagccagcttctcacctgcaggtaccggggagactcaaaaggcacgagttctgacaagaacgtaaacaggaagaccagggccttct

SEQ ID NO: 29 - DNA, 599 bp, Homo sapiens
gcctccgtctcacaaaaaaaaaaaaaaagaaaaaaatctgactataaacttgttccctttcaaatagaagtgcaagaggaaaaaatctggttcatctaagggtcctgattgggtttggtgaacgctagtgtagctgtggtcaggtgagcagtaacttctgcagcgttcctcaggttaggagcagctcacaggggtcctggtgggcccaagccatatgctcacccgcccctctgaagtggatggtctgtgccattcacagttgcttggagaccatgatgtccataaataactttggttttrtggtttttcagattttgttcaaatgtttgttttgaggggatttcagaaaaactcagatctgagaagaactgtggagcctgaaaaaatgccgcaggtaggaggaaaagtcaagtgaagagtttagcggaaggagccgtccttattccaagtgttgaaatgtatttacgtgtctctctattgaaacgtgctgtttgtacctatttacggggtacatgtgatacttgttttgtgcatagagtgtaatgatcaagtcagggtgtttagagtctccgtcacctccagtatgtatcatttctgtg

SEQ ID NO: 30 - DNA, 599 bp, Homo sapiens
gcctggagctcaagggtcaggcaggtgcaagggccagtcccacctccaggggagctccccccagccggaggctccttctgccaaagaatgttccggcacagcatgcagactatgggggacacaccagccctgtttctgtatgtcttctaacctgtgagtttgcctgttttctctctagggcaaccccgtggaactgataacaaaagctcgtctttttctgctgcagttaatacctcggtgcccgaaaaagagcttctcacacgtggcagaggtaacgtcaagatgcccaccttgggccaygtgggggacatgagaattgacagctgcctgggccatgtgcctgcctggctggcaatacaactcgactatttctgccagagcccctcccctgtggtctgaggcctagtgggcggcatgggctgaggacctgctcctctctcacagctgctggctgatcgtggggactgcgacccagaggtgagcgccgccctccagagcagacagcaggctgcccctgacgctgacctgtcccaggagcctcatctcttctgacgggacctgccactgcacaccagcccagctcccgtgtaaataattta

SEQ ID NO: 31 - DNA, 599 bp, Homo sapiens
ttggttcacatagtggcacccaactctatgctgtataaagaagtcatatccaaaacacagtgatccaggaaggctgaaaatgacaggagggtgtatagcaatgaagagagaaaaagagcttgggagttttaatcctgactgcagccaagctagtatcaggccaaaagcattaatggaaacaaagagggccgctgtcaatgctaaaggctgcaatttgcagagaagatacactaggtgtggttatgtctgcttcacataacatcaccagaacctctacgatgcggaaaccacaggggcggyggggagacatgagggacacgcagttccaggacattgtgactgccttgctcagcctgagacatgtgaaggggaccaaaacatacgcctccaaatgtaccagactttgtgctgtgataatagggaatacaactttgtttcgagactacttggaacacttaggaaagttgacatatattagcttacaaagacaacctcaatgagttccgtaacagaaataatacaaacaacattctgtgctcacaatgcaataaaactaaaaattaatcataagattaaaaaccaaaaaggcccttcaattt

SEQ ID NO: 32 - DNA, 599 bp, Homo sapiens
ggcacacgcctgtattcccacaccaggtcttccgatcagtggtgtctgtgaagggtgccgcgagccaggctgaccaggcgcccgggatccagctcatccccttggctgggaacatcctccactgacttcctcccactgtctgccctgaacccactgctgcccccagaaaaaggccgggtgacaccggggggctcccagcccgtgcaccctggaagggcaggtctggcggctccatccgtggctgcaggggtctcatgtggtcctcctcgctatgttggaaatgtgcaaccactgcttctygggaggagtggtgggtgcagtccccccgctgtctttgagttgtggtggacgctggcctgggatgagagggcccagaagaccttcgtatcccctctcagtcgcccggggctgtcccgtgcatgggttggctgtggggaccccaggtgggcctggcaggactccagatgaggacaagagggacaaggtatggggtgggagccacaattgaggataccccgagactaccaggagagccctgggctggaggctgagctgcatccctgctccccacatggaggacccaacaggaggccgtggct

SEQ ID NO: 33 - DNA, 599 bp, Homo sapiens
cccatcactgccagatgcgagcagttcttccacttggtcaactctgagatggtaggtgccccgtgtgcacgtgtccgtccgatctgtcagtgcctgtggcgctaacagcagctcttgcagcctccatgcgtgtactgaccgtgtgaattactgaggaaagggttcaaagtcaaatagggaacagtgtctggaatgccatgtgagggaggaccccacccacagccaggacggcgaccagcacatcaccaaaccagctgtaggttgccttctcccaagtttgagttcctttagtttccctchagttttaatgattttgacacatggaatcgtacagaccattttgtgtgccacgcacctcggttcatttatttctaatctcaacatgcttcatgtgtaaaaaaagccttgtggtgatctgactaccgctgttgcctgccaggcagcccagttatcttccattttagacactgcccagcgtgagttgcagggtgatcttaaccagcaggctgggtgcggtggctcacgcccgtaatcccagcactctggaaaggtgggggatcacttgagcccaggagttcgagaccagcctgggcaacatg

SEQ ID NO: 34 - DNA, 599 bp, Homo sapiens
ctctgtctcagtgaacaaataaataaataattttaacaaaatactgcatctaacttctggtctcacagttgagtccagcttaagcccaggcctatccaaagtccaccccacataggtagaccatactcaccaacctttctagaggacatagagggactaaattcccctcaaattttttcttacccagttttagtctagagtgctgtccagaggtacttgcttctccaggaggggacactggtttctgagcctgctctgagtcacttgtattcagtaaatttggcgttcctgacaattctytcaaactagattcattattgaatgacgccttgggcattccctttaatgtgagaatcacatattaaaagacaacgtgcatttatttagttctttacggcttgcaacatgttttcatatgactgtagttaatgttcaaaccttggaaatacctagagtgtctaaatactagagggaaaagacaaggtctaagtgggtagtgaatctgggactctaacccatgtcttaaggctctttccttcaacttcctctcaaaacgttctgtgtaagttaaagggaaggaggaagaatacttgggaaga

SEQ ID NO: 35 - DNA, 599 bp, Homo sapiens
agctgggatgacaggcatgcgccaccatgcttggctaagttttgtatttttagtacagatggggttttaccaagttggccagggtggtgtcgaactccggacctcagttgatccgcctgcctaggcctcccaaagtgctgggattataggcatgagccactgcgcctggcccagctttttttttttttttttttttttaaaggttgacttattcttatagcccacagatctgaagtaacaaatcaggtgtgggcagttcatatctgccagaggttctcattcatctttttttttcttttygtgggatggtttttttggtgagttgtttagtaccagtcttcccatttatgacctctaacaagaaacacagaaaagtattattgccaaagactaatactagttttcctaaagagaaagaaaaatgcattttactttcaactttttttttttttttttgagacagggtatcgttcatttgccaagggtggagtgcaatggtgtcatcttggctcactgcaacctccgcctcctgggctcaagtgatccttccacctcagcctccccagtagctaggactacaggcgcactcagccacaccc

SEQ ID NO: 36 - DNA, 599 bp, Homo sapiens
aaactcaactcaagagtcaaaagaaactggacctttgcatggtgggcgtgactggctgagaccctgcagggctcaagcaacattacctcagtaatgtccccagctgatgacaaatcctcgtagagtcccatgttttctatagaaaccttcagggaagacacagaatgagaacaagaaaacaaagcagtttctgctgggacaggtgtgaggagtgggcatggagggacagcttgccttgaggtcggccagccgtgtcttggccaatgagatgtagtctgtgaggagggagcggtacttgcygggaaccaggggtgggtggagaatgtgcacctgaggatagatagcagagcgcagcaccgttagtctgggaactgcctgggactccagggaggccacaattcacttccaacatccacagtgctgagtagagaaacacagccctttacagtcagacttatgagtatgcaaagcaaaccattagtaaaattaaaacacaagccgggcgcagcggcttacacctgtaatcccagcactttgggaggctgaggcgggcagatcacgaggtcaggagatggagaccatcctggctaacatggtga

SEQ ID NO: 37 - DNA, 599 bp, Homo sapiens
ttgctgtttgaaatgcagctttggacaaaaaataatgacactgtgtttgttctttctagagggtcagatatttctatattagcctcatcagtattttaaatttcgttgtcttaatgtttacattccttaaacattgggaatgtggaagtattcctttggaagcaagatcttctgcagtttggaccactgcgcgcagctccataggcgcacattagtgaagcacgagggcatcattatctcttttgagccttccgtgcccttctgctccgttttggctgaggttgcgcttgttgcagatayattggacacacttggggagggaagccagctgcaggtcatcatgtatgatctgtgtttgtgatattaatttgtcatgatttaccccatcccattttctttttagtagaaatatcttgtttaattttgtaatcctcttctttcctgtatagaaacattgcttctcagactgtagccagattgaaagatgttgcccgtcggatttcatcatgtctggactttgagcaacacagtcgtgaaagatctgcttcattggatttgttactgcgttttcaacgtttgcttattagtaaactttatcc

SEQ ID NO: 38 - DNA, 599 bp, Homo sapiens
ataaggggtgtcatcccgagtgttcctaggctggttctctgggctgtggccccccaggggggcagctacgtcggggtgcccaccagtcccgcggggccctggcccagtgtctgtccgatcacagctgtttccaagggcttgaagcccacgttgtccagagggatcccgaaggccagcagctttgcctcatacgtgcgcagcaggtcgttatgggcctgtggagacagggcagtgtcagggagctgagggtccccacgctcaggaccaccagagctgagtgcagctccatggaggggcagygtgagcatcacggtgcagaaggataaagccaaaggcttatcagtcatttcccgtgtgggacacagctccaagcaccttcactgcgttggctcaggtagcacgacagggagaggctgtcactattcctattccacaaatgacgagacggaggcagagggcagggacactcagggcagagctgtggtctgggagggctctgggccctggctgggtgtttcccactcaggttcttcacgccctcccttcccctgttgaatgacgtctgtttccttccccggtgggcacagcaccttctgact

SEQ ID NO: 39 - DNA, 599 bp, Homo sapiens
tgaaactttccccaaatgagattcaaatctgggcaaacactctatcccctgggcctcactcaccccacccgcccacctttgaagggccctcccagctgctgcgctggtgggtttacacacacctcaaaaacgcagaaatcggaaagaaaaatacgttccagtttagttccaaatgtttgaaatcagtaagtattcctttcacaaaaataaactgctcttcctgaagaggcctcctccccactcctcacggatgaaaagaaaccagaggcttcccgacaggccctgcagaaaacaaacagytgccactttccttttctaactaatctcttgttctgaaaactgctagccattttacaatttctaaaatgaatctgccatccgtgaaaccaccctgttacctacatacaaccatctatgtcacccagaaagctgagtttgtcataaacaggtgcttttttttttttttttttttggagacagggtctccttctgtcgcctaggctgtgatcacaactcactgcagcctcgacctcctgggctcaagcaatcctcccacctcagcctcctgagaagccaggactacaggtgtgagacaccat

SEQ ID NO: 40 - DNA, 599 bp, Homo sapiens
ccatttcagcctttctcccttaccgggggcagggcagcgacggcttccttgatctcagagaggatcacatggcggtggatattcctgggtgcacgctggtagagcaccttccgcctagggccggggagaaacaaggcacagcagataaagcacagcaaatgaaatggtaaccgccatggagcctcggcccccacccccacccagcacgaggcatctcgctgcatctctggtggctgcaaaaactgtacactttactctcgagcagaggcaccatggctggtccctggcctgcggctgggkagcctctggggaccaatcccagccacatggccagtgactcagcctcttcatccaaagaacaagaacgatgagagccgtagccagtggcttcttgtgggaagtcactgagttcatctccatgaagggacacctgggtgtgtcaccatcgtcactgctacggctacaggcacaggcactggtccctgctggaaatcgctcttggagagcagtacagggaacaaaatgctcctgtccatgagaatgtccagtccagtggggacagcagacacgcggacgggcccatgactgccagcggctgg

SEQ ID NO: 41 - DNA, 599 bp, Homo sapiens
gatgttcatggtgtccgagaggcctgctgcttcttgtgcctgtggccatacttagtgtagcttggtgtataattggtgctctggtttctgggcctcttggccctagtccttcttttactgtttcccactgctgccagtgttgaaagattttttctactatgctgcactggggaagagagtggtacttactgaggacccggaatgcacctggcgtgtaacatacagtagctctcacctcacggtaccatctgtagacagagtgttagggccacctcagacatggggacttgggtttagaayggcttacctacgccctcacagccaggacacaggtctcagactcaaatagccttgctcttcccacactatctgccccagaaatcctggagggcccccgttcacccaagtgagcctgtcctgggcaaacatctgccccgcagacttggagcgcaggggtcctctcgccgagtgcttcccagaattcacctgcactcagactcccacccacccctcatccagccacactcttgtttttttccctctgaaaccacatgtatttaattttattagggtttttctttttatcttttgtttcctgt

SEQ ID NO: 42 - DNA, 599 bp, Homo sapiens
ccccttcttagtttggcattggtgaggggatatagtaggggtggagatagcaaggttttcacttgagtgtcttggtcttattcaattttggcttcatgtctatatagctggaagggagaaaatgacagccgtaatgttaacctgcttgtcctacaacttccccctgggtactgtaaggaaattgtgctcagggtgcagctcacagacccttaggaatagagacagagtgcagtggaataaataggaagtcttgtcactagcctgagcatcgtttggtcagtgtgtgggctggactaaggycctttctttttatagccctgtttcctcataaaatcatctgggggggtccctgaaattctataaaggcccaaagagtgggggtggggaggtagggacagaaaaagggttggaggtccctgtctctctcacttctcccctctcttcgaacgaataagtagaagcaggtatctggccaagtattaatgttggtcatcttttgtttccacagccttgccttatttcctcagtgttagtgtcttctgtcatttcataatggctttcatggtaatcaatgtcctgtacctcctgggaggaaggaa

SEQ ID NO: 43 - DNA, 599 bp, Homo sapiens
gggacattccatccaggagcactcacaggcccacagccagctgtgagacaggagggagaggtcgatgttgggacagacagaacaggtggtaggagctgagagggggttgttctgatgggggagatcaggaaagccctcctggggagggggctgggccgaccccatcccctccctgccacggctaagccactaaactacaccagagctgaggtacagctgagcttaaaatcagagctaagctaccgctgacattacactcataaaagacaggcaggagccctgcatggaccaacagaaggygcggccgtcccgggcctgggaacttcctggcactccctccaatgtgagcatgccacagaccacagtcgtgttcatgaggcctctgccccatcaggagcagaagtcacaggtgccttcaaatcacgcggtcgccacagccggcatctccacgcgtccttcctgctcagcgccacgctgcagctgcacaggccttactctgtcacaccttggggtttgggatattctgttaactgctgctcgatgtaattattttccttgtggctccgtggatttggggctgatttaaagacattatttga

SEQ ID NO: 44 - DNA, 599 bp, Homo sapiens
tcatctaggcccttggtgttcacctccgcacctgcagccagcagctgcttcgcgacgtcgtagtagccccggttacaggcctcgtgcagcgccgtccagcctggaaacagacactcaggacgtacgttatttaatcctcagaacttccctcctgtctctggaggcagcgccctgaggatgtgctacagtgcggggacaagccaggtggggagctgccctgtagtgagggctgcagtttagggaagcaggtgctggcccagagaacccctaaattctctttactgtggcacctcgttagcrcgatccatatctagcttattaaccctacggactgacaacctgcagggctaatctgcggcaatcaccctaaaagggtaggaaatcagtgaacacacagtgtagtaaacagcagcctcatcctcaccgatctggaggcgtatgtgcctttgcaaaacacggaggggaaacctcccgtgtcggaaacggaaaccaaggctgagagcctcctgaaggctgaagagctgctacagcaggagctccaggggcgcccaggctcgggctgcacgacgaaggccaccccgggcaggcagctcctggct

SEQ ID NO: 45 - DNA, 599 bp, Homo sapiens
aaacaccacagggcagctcctaccatccctgcataaaagaacaggcagctcaggtggccgtgacttacccgtcgagctctcctcgctggaagtgtaagtgcctttgcctaacaggaggttcaccatcgtgggggagttggccactttcagcggcgtctcgcctttcctgttgctctgctgcgggttccctccgtaccgcagcagcagcttcaccacctacaagacagtaacacccgcgtcagggactgctggagaagcacaactcctctaccgttccgcataacacggcagccccttccragaaccctggggttcgacccgagagcagcccctcacggtctgagggtgtgggagccgagcgcccagggactgcctggcgaggctctgggtgtggtgggcgaggctgtggctcctctgtgggaagggctgcagccgcgggggctcccgcacccctcctgtgctctccttcagaagggctctgcctcctttggatttttttccaccatttctccgttgccgcaagtagacgacttcccaggaatcttctcacgaaggctctcccctcccgccaacagtgagctcctcagcagagtggtgcc

SEQ ID NO: 46 - DNA, 599 bp, Homo sapiens
agcaggtgtttccatccaccatccctgatgcctgaggccagacatattttctctctctacctgaaccaatgaattttacattctcttccaattttccaccacaaaaccggaatgtctcagcctggagaaagggcatctcagcacctgactcggaaggctgttcaagctcatggctgtctagaaactcagtggtaatgtttacaagtttaaggattatctggaattaacattttaaaaaatccagactgtctgctctcaggacggaaaaacggggccttcaccatgtagagtccaccgtcytgatcccagatgtgagtctgtggcccacggacacactcaacagctctggcgtggtcagggcgcagaccccacggcctccggacggtgtctgggccccgacagtgcggggcttatcacttcactcctgagacacctgattctcttcatgtccctagcttggtgctttgcccaggcaccttccccaatggaccagggatctgggagtccactcaggaggtggaggaagtgggtcgcacccaccaggcaaaatacttttcacggcctgcatcacagcgaacactccggggactcgggagtgt

SEQ ID NO: 47 - DNA, 599 bp, Homo sapiens
agagaacagccaagtcaaggaggagctaagccattttggtaatatacatctcataggctctttaacagttgagaaaaatgtttcaggtggtgagtgttagcacctcttttacaatcttgtgacaaagaaagcaagttccagaccaaattctagcacacctaaagacatctattaaacagatattccttagtgagccaggcccaaagcagttcacacaaactattctagaagctccatgctgtcccagctctttctgcctctagttattccaaccaccacttatgaaaaaggttcactcayaatagaactgacaatatcccagaggcagggttctgttgcccttggagtcacatcaggcaaccagaaatcctaaccaaaggccaaaggatcattaaggaattacgcctcaggcgccatctccaagggaagaagttacaggccctgctagattcatattccttcttcatgcaagcataggaatatttcagggtctcgctttgttgcccaggctggtgcagtgacaggatcatagttcactgcattatttgggccctaactcctgggcccaaataatcctcctacctcagactcccgagtag

SEQ ID NO: 48 - DNA, 599 bp, Homo sapiens
tggcctacaaagaggattactaataaaaatcagccaactcaccccacagaacttctgagaaattcagcactagagtgcatcaggtgctatgggaggtgggggtatggcatagagaaaaaaatcaagttgaaagtttatatacagaccactgagaacaacaggtgttaccccaactatgtgcagccaaatgaccattcccgcataacccatccctctaactttctactctacctgcagaagacctcagttctggagaaagctaactaagggcacaagtctaggaactactttgcacagtartgggctgaggagaggtgtgggctgaaaagcaatgagattaaataaagtctccagactgaaaccagccaccttccctgttcctgctctcacaactccagcagctagtcttatgcaccctccaccccacaaaaaaaccctaactctgctaccccagacaggacttcagaggattcctctttgggaaaactgaatagttgcaaaggaaaataaaacctgcagatgatgacatttgtaaatgcccccgacccaaagaaagaggccagcccactgcctgattccctgtacagtgaaaatgatcc

SEQ ID NO: 49 - DNA, 599 bp, Homo sapiens
ttaacaaaaggaagttgcactggataagtacaaagattttcagcagccagaaaaggctccttcccgtctgtctgtctgtcttgaagatggtagttggacatggtgaaggcagacgtgagggtttgagtcccagctcaccccttactggccgtgtgaccctggacaagttacataatcttgctaagcatcaggatttctcttttgtaaaatgaggatagcagctcttcttacctgtctcacagtgctgctgtgtgacaccacatgctgtatggaagtatttgaaccatacggagcccgtgrtagctgccatcatcatcatcatcatcatcgtcatcatcatcatcatcatcgtcatcatcatcatcgtcatcgaggtgaagtccagggtaccctatgttgggcgctattctaagcacttttcaggggttacttgcttctcgtaacaactctgaaggcaggtgctgtcctcagtttgccgatgaagtgaaagaaacccaaactagtccaagtcacgcggccaacaaaaagcagagtcagtggtttatgtgtttttaattaatcgctttatttttagagcagttgtgggtttacagaaaaac

SEQ ID NO: 50 - DNA, 599 bp, Homo sapiens
ggaggatcacttgagcctaggaggtcgaggccgcagtgagctatgatcatgccactgcactccagtctgggcaatagagcaagacccatctcaaaaacaaacaaaaagaaaaaacacctgggctcaggtgactcctccagagttgctgaggtgatagacatgagcaccatgtccagctcctgctgcttctggaaagttctgtattttatttctctaacagttctgtgtactctgcagtgacacgtttggattatgggcattagcaaccggatgcttacgtttacccagaaatggtacaaygtttgtgataactacatcaggaattgaagttcctgaacaggtttctgccctgtttcagagcacagggtctgagtcccttccagccccaacgcggctgctgcttcgacagtctcgtcccacactgtcacagccacgagctgcggccacatgtcctagccttggccactcacacacaatgccgtaggtaccctctccaatgcggttcagcttctcaaactccttcacactccggcatcgtcccagctgaaacagaagggtgttgccagtgtggaaatttggcagctgatggaacaactgag

SEQ ID NO: 51 - DNA, 599 bp, Homo sapiens
ctgcgccactgcactccagcctgggtgacagaatgagactccgtctcaaaaaaaaaaaaaaaaaaaaaagcacacgtagcaaggtttgaaacctcacagcccccatttcccggcttcccttctcttaaccctcagtatcaaacctgggctcaagcaatcctcccacctcagcctcccagattgctgagattacaggtgtgagccactggccgcatttgtcctcagtctgattccttcctgacacctggggaccgagtcagaagcatctggcctgtcactcactgatggcctagtctcaamctccctttcccgccttaattccccttctcacatcctctgcttgtgctcttctcgacacgcttatttctgggtgtccaaatccttgtcacacttcctgaactcaaagaaatgccaggccaggctgggggcggtggctcacgcctgtaattccagcacttgggaggccgaggcaggaagatcacttgagcccaggaggctgacgatgcactgagccgtgatcacgccactgcattccagcctgggcgagagcgctagaccctgtctcaaaaaaacaacaaaaaagaaaaggccaactcact

SEQ ID NO: 52 - DNA, 599 bp, Homo sapiens
gaaataatgctaaatgcagttaagaattctcattttgcacttgtgcatttgggctttgttaaagaatcaggttcgttatcacagaggcaggtcagtgctgcaggtgcattagacacggtcaggaatcgtgcctcattcacaggtatccctggtgcctctcacagtgaggagtgggtaaaaacacactcaaaaataacgaatcaacaagtgaatgaacaagcagctaaagtatctgcattaaacacggaatgcaaattctcagaatgccacacaacttggatcttcttgtagcaagtaacrtttactcctcattgcaggttttcctgtggtcctcgggaaccacccaggtcactgactgcacaactgcagtgagctgcattccatgtggatggagcctggagaggcgggccacagagcagcctgaccctgagaaacacacccagctcttcttggacatcacacagctttgccgttgggactgaaccccctttcttctcaggctcctcaaaatccaaatagaatgcatttctgattgatacattccatgctcaccagcatccaagcagattttagtcacccccactggtgggccactatgt

SEQ ID NO: 53 - DNA, 599 bp, Homo sapiens
actctctggttgtgaagggcagagcctgactggcatcctcggtcatgaaaccttttgatgtggtctgcacaattcagcctctggggcagagaatgtggggggacggtgaagagagagggaggggaaagtcagcagcgtcccctcccccgccactggcctgtctgtattggtggaaatctttatttgaggcaaaatgctcagaaatagaataatctttattctttccaacctcaactcttttaaatgactcacttgatgtaattgaggcatttcctaagccagattggaaaccacgcagamgcttccaggtgttgaatggaagccattagcttcactccgagctttggtgttgcagctacagcgggtatcttgggcagtcacagccactttctgggtctaggagggagagggcagatctggagctcaatcttcctggttctaagcctgtgtcctgcagcacacagcaggtgcccataagagccactgagtgttggagctaggctttaaaattgctgtgaggattgaatcaggtaatacacacaggcctgaagagcagtgcctggtgcacgattgcatgagatatgaacaaacagttcagg

SEQ ID NO: 54 - DNA, 599 bp, Homo sapiens
tgccctgggagcctgacaggcctccttcagacaacaattcagggacaaagaacttcaggtgctacaattacaggcttctgaaatagaccctgcaactaccgaaccgacaccgcctggctgctgcgggagacactgaggttccgaaagaaaggtggcttcgctattcaccggctaaaggagagtggtgctttctccagaagctcctcccctcacagcagacaccctgggtgtccttcacccagcctcctccacagggcaggccagagggccgggaggaggcctcgagctccttgctcagargacagccctgaggacattgctgtgggcctttccaaccaggccgaggcccctcctacctcacgcctatactgcacgccctttctcagggaatcaccttgtggggggcagggtccagctggcttcccctcctccccaggcaggcctcaggcctgcagctttcacaaagctcagctttcacaaagagagggtccgtgagtttccggaaagcgtggttaatccctgggaacactgcccttttccaaggagcctgctggaggctcaaatcaacccctcatcgtggctggtgacccactgcgctg

SEQ ID NO: 55 - DNA, 599 bp, Homo sapiens
aagaatgcagtcctggctacaggtcccagctcctgcttcactgagcaccctgagggagcccttcctaagcaaggggagccctgcctaagcactggggagcactttatctcacaacaggaaaaggcattctttcccctcagtttctcagagacccagtgacttcaggatttgcaaacaattgtgttactgaatgtgtcacactctgctccagctgcagggaagaacctgagtgatgagagtacatgattctgactctgaccctgacttcctcttaacctccatacccaaatcaagacctgygcatggagctggctcagaccggggagccagatgctgtctgaaaatatgccatcaattctttctactcacttaaaattcaagtacctaagacattcaccccccattttgtaaaaaagcaaacaaaaacataagactcagagacagaatgtaagaatacaacgaagtatacctggctggaagaacagcttttacagtgacagcaacaccctaatgggccctcaagacaaacttgtgcctccctccgacggtgtacaaggagttaggactttcctgtgaccctcaacaaatacttgcaagta

SEQ ID NO: 56 - DNA, 599 bp, Homo sapiens
cggaggcccgggagggacctgcactgctggggtgggtgttacgttcctgaggccccgtctccccagagacccgtctggcaccctcctcctgtccccgtgtctcactggagaggtgggctccggtctgcttggcagcttctctttgactccagttgcactctgagcttgcagcatgttctggaaagctctttcatggtcactcagggatgtctcagattaactgtcaggtttggtggatgcaggttgggtggatgcacgtttggtggatgcagagtgactgagctggaagatgtatcaggrtcccaggaggcaggaccgtggctggggatgcttgggcttctcccaggaacagttgggcatctcagccctggagcagctgaggcctccggtgctctgaaggcttcgctgaccactgcacggtatttgccggcagcttacctagggaaagttacagaaagaaggcttactgatggggaaaggcagtgtgtgtgtcgcaccgtccccctccccacgtcccgagtgctgtgtgtgtcgcactgtccccctccccacgtcccgagcgctgtgtgtgtcacactgccccctccccacgtcccgag

SEQ ID NO: 57 - DNA, 599 bp, Homo sapiens
gctgtggacgtcgggggtgctcctggggcagggacacagccacggccctcacaccagtggagccgtcttccttcccgaggcagaggcttggccttctcacaccttgggacccttctctcccctgtgctcatgctggaagcacagcgtgagggggcaaaggtcatgggaggcaaaaggctgggctgaggccgaggctctgtggctgtggccggataccaggtcctgtggtggtgtgggcagggcgcgctctctcctctgagcagcctggggctgcgtgtgtgaacagaaacaggcctgccratttgaagatggtttgaccaagtctcccaaagctgaagagaggtccctgcccatgtcccccgggtggcaatccacagacgcacctgtgtccatcagacggtatcgttgatagggagtctgaagccacccagacgtggtctgttcacgccgtggacaccacggtctgaggcagacggttacagccaccggcataggcgaatctcagccagagagagtgaactgggacaccattcacgtgatgggccagatgggctggcagcggctcgctggagccgtccgggtgggaacgctgtgtctgt

SEQ ID NO: 58 - DNA, 599 bp, Homo sapiens
gtttgcttccagggagggcgcaggaaaggagtggcctcccctttctggcaggagagcaggtccttaggagagaaacgggctcaggctaacacaacagattcccctaccaggaaatacgagatcgctgatttgtgccaacagcgtttaataagcacttcctggatccctggctctgggctgagttactgtttacttgaatcacatgaggaaacagttgaggcagaagtcaggggtcagaggtcaggtcaggctcaaagccacagcacagggactggatgccagtagcttctcccttgaccrtggtgccctgcaggtcagctgggtggccacagagcaggaacccacgaggggcctcctcattgctggaagtttgcacatatccacagcacacgtgctcacacacacattcatatctgcacgcaggtgtccacacggaccctgtgtgtgtgcttatgtgaacaggcataggatgtcctgcacggattatgacatacatgtgcacctgtgtgtacatagaaaggtgtccaggccagacacagtggctcatgcctataatcccaacattttgggagcccagggtgggtggatcggtgagcccg

SEQ ID NO: 59 - DNA, 599 bp, Homo sapiens
acattgcagtggaaagttctggtcctgactgctcatagtggtgaagtcagtttgcgccttctcggggggacggcagtggcaggatgctcgttacaggggctcttggtgtggctcaagctgcaaagtggcttcatggcaaagctcaagccagacgccagggcaggaggacccaggccagggttccaagggagccgagctggctgtgctcaccgggctgtgggtttttccctgcagagttggggtgaagccccagaacttgctgcaggtgcttcagaaggtccagctggacagctcccacaracaggccatgatggaggtacccgccccctgctcccagcctcctctgggctcctcccgggaggtcacttgggccagcagccccttttcaggactaggggtgtggcttcaggtcacctgcagcattcacagcccgtcagggtggggttggggctgggggcgctcaccttcgagggcgctcaccttcccgtgcgcccctgctgacctgtgctccttcctgtgcagaaggtgcgttcctatggcagtgttctgctctcagctgaggagtttcagaagctcttcaacgagcttgacagaagtgt

SEQ ID NO: 60 - DNA, 599 bp, Homo sapiens
ggctggtcagtgccctcagcatcggcagtgggcttctaagcaactgaaaactcttccggccgactgaggaagagaaaaggttaacaggaaattcctcaaactgcagggcagtgccccgaagcccacagtgaggacagcgagggcacctgcctcgcctggactcagcgctcacccgggacacatgatgtgacttttctgcccacagacctatggttcggcctcacagccatcaacaaagtgtggggccggcagggcctgggctcagctcaccgcaaggagcctgggcagtgcggcagcttytgcagagggaggggttctgcgtcccattccccaagtgggttctgatgttggaaagtcaaaaagaatgaccaacatccactatcaatgctgagtttgcggtccatcccaccacaccatcgcctgaaagaacatctcatttgctctccgagtcaaaaggaaaagagcaagcagggaaagaaatgttgtcaacaggtgatagaaggcaaaagttccttaacatataaagaccagctgcagttcaagaaaatgatgaacatcacaatgaaacacgagaggaaggaggtaacggggccccgcgg

SEQ ID NO: 61 - DNA, 599 bp, Homo sapiens
tcgcagtgggcggtggtttgggcccgaccgagctggtctcgcagtggtttgggccctgcttgctcgactggatttcaacagtgaagccagattagaggacatgctggctgccccgatcctggacctggaccaatggtgcttcctaaacattccatgtcaacttttctttttccaccttccagggctgggctgtgaaagagattggagaggcaggtgcctgtgagagaagcttctaccagcctgggaagtcaggtcttccatgagtgaggggaggtatctactttgccaaaccttgttagrgaatgctgacatagctgcctcttcctgcccagaagctaagcttgaatgccacccctcacagagcttctctcctgtctttcactcactcctgcttatttctgtgacagaacttgacagcatggacattgccacttgcttccttcagtctcctggagtccccatgtcctgaacacagaggaggcacccaaatgcttgctgaagccacatccctggaccaagtgacctgaccctcacctggaggaccttcaccttcacctctcacctggaggaccctctcacctggaagactctctcacctg

SEQ ID NO: 62 - DNA, 599 bp, Homo sapiens
ctacggcccttccaaaaattccacaggccatcaaatgcacttcaatatactctttgaagtatatcccaggtttcaatcaggcagagtagactatattgtccaaaattaaatcctaagcaaaacaaggggaaaaagcccagtaattaagcatttatttctcctaagggtggtcaaaattttccttagaaatttcccattagtgtttcacaattcaagttgaaaaataaggattattttaaattttaaaaaagaccaattttttataggcaatccatgagagtttaatacaattctaccagmtgaaacacagcaaagcatttacagttggcatgcctgggacaagttcctctggtaacaataaacttcaattgcagattcattgacatgcctgagacacatttctctggtaacaatactctacaactgcaggttcaatgacatgcccaggacttgttcttctgataacaatatatgtcaattgcaggctcactgtttatgaagatgatgacatttaaaacagagaacgttatcaaagtaagatatggattcaaatcctggtgctgtcacgtgttgtaatccagagcaaattatgtaacctc

SEQ ID NO: 63 - DNA, 599 bp, Homo sapiens
cgtctctgctgtcctcttcccaataatgtccacctcaaaacccacagggagaaggaagggccccttgtctaacacagcagtcctcaggatctcactgactaccttgcaggtgcccttcccttaggtgccaagagttggcacagcctagcaatgcacgtcgtgcaggacctgaggtgtctctgaaggtggggctagatctgacagacagatgggatatttctgagaagcgaatcccactacaacggtttgtgttcaaaaatttcaattaggttagagaacaaaacagcacatataactgcrgctcaagataagatgccctggccccgcacaccacagacaaagccttgaggggcccatgctgcagggattggtccttggccagaactttgaaactgaacagaggcaaggagcgtctttaagggacagagacaggaccgagcacccctaccactcactgcgaatcttggaagtgcccacgaaatgagatgttttccacccagtcacttctgcaaaggtttaaacctgtcagatctggtgcaggtgaggatggaaacggcccctccgcaccctggcaggtgtgtgactcagagcagccctct

SEQ ID NO: 64 - DNA, 599 bp, Homo sapiens
gttcaagtaattctcatgcctcagcctcctgagtagctaggattacaggtgcgtgccaccatgcccagctaatttttttgtatttttagtagagatggggtttcaccatgttggccaggctggtctcaagtggtccgccagcctcactggcctcccaaaatgctgggattacaggtgtgagccaccgtgcccagctgacatacttttaaatgttaggatccctttacctctgtgcacctgcacataagcacacacctcaactgcaataagaagcccctcctaaaacacccaaaaagttayagaatgtaagaaggtaggacttttagaggggaagtgaagggttttttggcacgaggctactaacgtgcaatgtttaaaaacaaaaacctgtaagggtctttaaaaactgcacactatcttggaaagtataggaatggattctgcctgaaaggaaatggatgaggtgcctggtgaggcagcatcatccaacacatccaatcaaagccagtggagcccctcatctgccttgctcagaccccagagatcccacatggtcctcacctttatgctacaggaagtgcagacggctctggaaggga

SEQ ID NO: 65 - DNA, 599 bp, Homo sapiens
gtcgtcttgcttccaccaccaggggatgactacgttgaagggaggcctgtgtgaaccgctgggcttccgcagctcccgcgtgtggtcagcgccgacgctccccatcaacgctttccagcacaggagccgggacctggacgtccatcgccggcggtgcctccagtgctctttgacttgcgcccttcccgggggctctggggtccgcaggacaccgccatggaccggctgacgtaaccaacaaggcttgggcttttctcttccccagtgagatttccccacgaccggcacagcgtcggcccyatcctgtgcgcttccatgaacggaagcaacaaagcctggcctgggacctcggcagggctgagcatgcacagctgtggctctgcataaagggacttgtttagagctggcactcaacaagaggccttcagtcagcggattcccaggcctgagcaaatccgggggcggaagcagctattcttatgatggcacagagaggttatgtggagaagatcgtttgatatacgcttgcctttgggctcaagtcactcaaaaaggtggtctctatcttcagtgcaaaaggcaaccccagcagagcgaat

SEQ ID NO: 66 - DNA, 599 bp, Homo sapiens
cccggggtcaagcagttctccctgcctcagccgcttgagtagctgggattaccgggacgccccaccacatctggctaatatttgtatttttagtagagacagggattcaccacgttggccaggctagtctcgaactcttggcctcaagtgatccaccctcctcagcctcccaaagtgccaggattataggtctgagccactgcgcctggccttccttttagatttttaagtcactctggaatctttggtgctcagatgtaccaaattgtaactcctcttgaattacatgcacaggccagkctagaaatgtccaaatcagtcagaaaacaaaactcggatctgcacagcagagtggatgcagcttctcagacaccgttggagtctcggtctgcactgtggggctggtctgaggatgaggcccgtggtttagtgttgaatatgatactcattgctttagctgtgggctggtctgagggtgaggcccctgtttactgttgaatatcatactcactgctctagctgtgctgattatttttctctttttcccaggtggaccggctggaagtcgtgaacaaacaatctgtgcgtgttattcctgc

SEQ ID NO: 67 - DNA, 599 bp, Homo sapiens
gacctcaggtaatccagatccaaccacctcggcctccgaaagtgctgggattacaggcgtgagccaccacgcccagccccatcttttaaagaagaataaaatctggttctacatggatgagtaaagcccagttaacaagtgatataagaagaaactgtttcctaagaaagagcttcaggaagaaaatgtactaaaatttacagagatgaactcgtttattttcaagcattccacaaggtaacatgttttatgtaagacatcctcatattcaacacataccttattgttctgcctgacttygcttcagcaggaggagtcgccgtgggggcggctttggccatcattatgggtgctgcagtccaagagctctggaggccaggctaactgcaaaagcaaatgtacttaaaaaaatttttaagtttagttgctgccaaatttggaaagaagcaaatatgtaattatttgttcagatattaataaggaagcctagatttttttttttttttttttgagacagtcttggcctggcgcgatggctcatgcctgtcatcccagctactcgagaggctaaggcaggagaatcgcttgaagctgggagg

SEQ ID NO: 68 - DNA, 599 bp, Homo sapiens
aagaaaagaaattaagctcttggtcgaccaaagatgagtcatagtgtatttcagtatttaaattttacacaattttatgttttagtcttacagtcttgtttttgaagctgcaatatttcaagttactaaataaattgcctcagtttctaagccgtgttttttgttttttgggggagttttttcttgctgttggaggctatagctataaacatttgcagtgcatttctgcagtgagcttgctcctgtttactctattgaatcatatttctaatggggtatttaaggcgataagctcacacytcaccctgcttgctccagtggttatggccagtgagaatgtgcggtgttgcattgcagaagaagctaggaaagctcatctggcctcctgttccagtgcttccaggggatccacgttcagaagctgactctgcaggcaagaccgacgttattttcctggtggctgtgttcaaaaccacccaaaggtacctgtctttcatccaccatcattaacagcatgaggtttacaggaactgagtggcttcctcagagtgtgacttctacagtgagtgaccagggggcgagggtccagacaactcagg

SEQ ID NO: 69 - DNA, 599 bp, Homo sapiens
gctacgttgattgtgttctgagaacaacaacatgcagggctctttgttttccatgaaggttgggggcaaggttaggaagaaggcaagaagggtgtgggtatcctaaaaggcattattgcaagggcctcatggggcctccacggtgtggggagagagagaagaaggccttggagtggtaaagggaattgttggacaaccatccaaacttggggaaagagacaagggactttaacaatgtccagtcaagggatgcattcacatgtatgtgagtgtgctcaggcaaatgcctgtgtgttagcytttctaccacggctttttaaaatacttctcaaaatgagaataaaatattatttttatagagctctttctagcttacgatgtgctttcgaacatattatctcatttttatgttgagcgccctgtaattgcatatgctgctgaaatagctattttaccatgttcatttcatgtggcacaagggtgagcctcaggaaggtggtattgccaagtgctatgtagctcccaagtggctgctctgaaaagtaacaaggtctttaggttcgaagtctagagctgtttctgtgctaaccctgccctcc

SEQ ID NO: 70 - DNA, 599 bp, Homo sapiens
aattacatgtatattttaatctaataatgactgttcaggaggagctgaggagggggcagagccaggttggccaccccagcagggtccctgcggtgctggtacatcagccttgaatcggctgtggcccccacgccaaccgtcctcctccctccgaggggacggggcacccacctgccccagggtgcactccatgccccccaggaaatcgtcctcctgacagctgaagccgctgggcccatgcgtgtcgtacacctcaaagcgcagcctctgcacctcctcgaagtagtagtccaccgtgargaccttggagaacacgggatgcaggctgctccggaccacctcggttctgcccacctgcaagtgcacctgtgagccgaccccactccagaggcttccaccccgggctccagacctccatctcccctctgccgctcacccacgcccaggtacctcaggaggactcataccctgggaagacctcctgcctgctctctgtccatgggaccacaatcccgagggccaccctgggggtcacccctggccaccactgtctgctccaccctcagatcacagatggggaaagtgaggctcacagagga

SEQ ID NO: 71 - DNA, 599 bp, Homo sapiens
gcttcctagcaaggaaagacagtacacatattgaagaatttgactgcgttaaaatgttaaacctctccccaaactcttataaacaattgtagaacaaatgataagctcaaaaaaatatttacatctatgagaggccaaagaatatcagttgactctggatcaggggctcggacacgttgctgtggaggcacacatcttcctggaaggcggtttggtaacaaggtggcaggactcaagacagcccacacgatccctgcccctgggaaacagccatgtgtaaccctcccctcgagtgtgggyggaatgaggaagagggaggtgtgtctctcctgcgattagctcactcatccattgagtttgacgtcatcaaaatgcgagatgatcctggatgggcccgacctgatcagggaagccagacagtaaaagtgtcagaggctctttagggcccgacctgatcagggaagccagacagtaaaagtgtcagaggctctttagggcccgacctgatcagggaagccagacagtaaaagtgtcagaggctctttagggcccgacctgatcagggaagccagacagtaaaagtgtcagaggctttttag

SEQ ID NO: 72 - DNA, 599 bp, Homo sapiens
gtcctgagataacatgagtgacaggccttggaaacagacatgccctgtcccactcttacctgatctgttaggaagggtgcagtattctaaagtgtactgcagacgtgtgtaaacgccatgcctgacacacactcaagcccccacgccagccggagcaggacggggcagcagcagggcggctcgcttgggggcggctgcagctggggcctctgagtcgagatcagggctccctggtcggagcctcagcccaagctctcctccagcctctaacccacaaccctctctcccttcccacagcaycagagcgtggggttggtgacacagcgtttcggggaccgaccctacctccgtgacagctggtcctcctggctgcgcacagagctctcgcccacggcagaggcgccctcgggcagctggctgagctggtccagcacctccaggccattctcctcggcgatctgcatgatgaggctgtccacctgctcctgcggcgtggtcagggtggtggccgagctcatggagtcctccatcacctgggggcaggggcatgctctggacaacaggtggggccaggcccatggggccccactcagcttcac

SEQ ID NO: 73 - DNA, 599 bp, Homo sapiens
cctacagacctctctttctgggttggtgagggccacagtgggttccttggccccacaggacccctcccgtggtctgagagcccccggggaggcgtctgcagggggacagcatgtacacaaacgtggagttgggaggcaggctcgcgggcggcaggaccgtgtccccttgcacacaggcctgtgcctctgctgccctgagcctcggttccccacctctggcaggtgcttactgccgtggtaccaggcctgggcacccccagtcttttgggcgccagtcatgtctccctagaagtggccccygttgcagccacaagaggtcagggcctcctggtgacctcctgcctctcttccagtgacaagaggttttccgctttggggtttggagcccggatccctcccaagtatgaggtaggagagcccagaacctgagacctcagagctgtgcccttctcgtgccctcccaggcagaggacggcctggcccagggagccctggctgcctgctgggaaccggagactgtaggatgggattggggtgcaaagcgggaaccccagccttctagccagagaggctggcctttggcctcctaggacttgtta

SEQ ID NO: 74 - DNA, 599 bp, Homo sapiens
aataaatcttggtgtcaggaagtactagccctttccatttgtttttgttttctttttttgtcattctggatactctgaatttctgtataaattttagaatcagcttgtcaatttctacccaaagagaaaacctgctatagttttgatggggattgcttgaatttatagattaatttggaaagaatggatagcttaacaatattgagttttccagcccataaacaaggtgtacctcttaatttatttagatcttctttaattactctgcagttttgtagttttcagtatataggccttgcrtatgtttggtcatatgcaatactgaatacagcatatgttttgtgctactgtaagtgaaattttaattttcagttatttattgctaaatatagaaaaccattaatttttgtatatggactccgtatcctgcaaccttgtcaaatttatttatttgtaggagctcttttgtggactgcaccaaatcttctacgtggacgattgtgtcatccgcaaataaagacttttaattcttccttaccaatattgatgctttgtatttcttttcttgcattatcacgtgagctacagcctccaataaa

SEQ ID NO: 75 - DNA, 599 bp, Homo sapiens
caggcacggcactcctcattcctcctcctctgtcatcgctggggccagcaggactgggggccgatctaaccggcagacggagaccaggttgtgttttttctcgtgggtgaggacggtcactcattagatcatctgctcattcagcagcaaacccgatggcgggacatgccgtctcgacacaggccctgcgtgggggtcctggacatgccgtctcgacacaggccctgcgtgggggtcctggacatgccgtctcgacacaggccctgcgtgggggtcctggacatgctgtccccagggtgygggggtgggggggaaggaagccttgagctgtggctccagggggtccgccaagtatttgttgagggagaacacagagatccgtgaacaattgtgatgggctgtcatgggggtatgggaggcgtctctcccagactcaggaagggtagatcagggaattcctgcctcccagcaggaatggccactcagcagggacctgaggctaagaggagtttgtgggcagaatgttctgggcaggggagcggtgtgtgcctgtgaggctggagctcagagaggcgatgggggcgagcagcaggcacccc

SEQ ID NO: 76 - DNA, 599 bp, Homo sapiens
cttcctgggtgctgctgcctcgaagacgggtggctcccctcgggcccctgcctgtcctgggctccctgggtcctgctgccttaaaggcgggtcgttcccctcggatccctgcctgtcttgagctccctgggtgctgcctgccctgtggccacgtcccactgttgcctttggacaaagctctcctagggcctggggttctcccatgagatgacaggggttggttcaaaggtctctgaggtttttagagctttgtgaagtgttctagaatccattttctctctcattgctggacagagagtrtagactgggctttttcttgagtttctctcaactcttctttgtccatgaggcaggaagaggggcttcctcagtcctcagcttggggtggattgggatggacagagaaccgtgcaggatcccagcctgagacggtccagcgccccgggggagcggagcccgtggctcagccgtctgtagccacggccggggtcactgtcactgagggcacggaggccccgccggcgaggtccgaggtgggcgtgtggggtggtgggcgctggaggcaggacctctgcttgtggagggtgggccaggcgtgg

SEQ ID NO: 77 - DNA, 599 bp, Homo sapiens
tgcctggggcagtgggcaagaccccatggaggctgagggacacactgcccctcaggcctggctgggaatcccagcacgcacctgggcttgctacctcagtcttcccatgtgtagaagggtgaattatggtgctggcccacggggtgtatggagaatgaaaagagtggaaataatgtccagcgtactgcctggcacaaggttacccgtgatctcagtgagctcttcgatgccacctcaggcctcacctcaccaccaccccccgccctcgccctggcacagctgccagggagaggcagaaamtcactcccaccccatcagggtcacaggggcctgaggctggagggagggaggaaagagctcctctcctcatccctgtattttcagaggaggaaacgggctgagagaagcagaagagagagcccctaacctcactcccaacccgggctgagaggagggtcggcagcggtgagctccgccctccagcctcactgcctggccccttagtccccgccatctgtctcagggcggcactccccctcccagggcctctctcctaccttgtcctcagctccgcccactgctggccgtggtcctccagc

SEQ ID NO: 78 - DNA, 599 bp, Homo sapiens
gaggcttttccagcagtttccagctctgctgtagctttcgctttctgtttgcacaaggcctcaatcagccaaggggagatcagagaatgttctgtcccttcctggccgtgcgtacagacccacacgtgcccagggctttccgtgtccccaggaaaatgtcagtgctgcccgggtccccacaggcgcctcgttcctcacctcgtcctttctgggcaggtgccggctgccccagtgggtagtgccttggacagctgtgtggttaaacggtgccactgattgtttctgaccaatgctgtggaragggggcttttctcactgagcgaactgcagggcaggccagatatcacaggcctatgaatgctttccagggagctgccagaggtgagattgaagcaggctgtttcaccggagtcgtaatggggtgcctggtttactcagcctgccgccctcaacccctcgcggagggagtgtgtgggccaatgaggcaggactggagtgcaggatcgctggaactggccggctgtttaggcaccggtgggttcaaactccactcacttggaccctctgtgctccaccccttgcaggagggtgagcaggtg

SEQ ID NO: 79 - DNA, 599 bp, Homo sapiens
ccaagcctttctgttggagcgccaaatcacgggcaaactgggcttcaagaacccttcagagccttgcgggtttctgagcggtacaccagtaagggcttcaacttaaagtccccagctgcactgccaccaagcagcagcatcaagcgatctttggatgacttaaaccctggctcagcattctcttcctcggaaataaacgttcctttgggcattctcttccaatcaagtctcgtctcatctacgttaaagacacctggggggtgtactcaccttcttcgatgatgcttttcagcatttctrtgtacatgtccttgttgctgggagctgcgctgttcatcttgaagtggggcaaacagtggcactccttgaatctcacaagccacccttgacttgcatgaaacctctccgtttgagaaccttcgccatgttcacgctgtaagtcatcaaactcagccttctcctgaacgatggcggcgctcaaggtcgcatttggctggctctggtcttccagccacaagctcagcagctgctccatgctctccatcactgcacttcgatggcgagtcaaccgagaggctctcagaggagtagctatttgt

SEQ ID NO: 80 - DNA, 599 bp, Homo sapiens
cacctcgtcagtgccacctacaggtgcaccctggacctgctgacacggtgccgggagcatgtggagaaggtgagtgtggcctctgcccagacaggtcgtgggtcccatgttgagtcacggtcatgccacacccgaaggccacacccgtccttcagcttgcttaggtttgggctgtgatcatgtcctcagagccgtctgacaggtgggccgttgtttccaggtgggccagcggctcctggagaaggaagtactggagaaggcagacatggtggagctgctgggcccccggcccttctcggmgaagtctacctatgaggagtttgtagagggcgtgggcagcctggaggaggacacatcccttcccgaggggctgaagggttggaaccgcggacaggaggaggaagatgtggagagcagcctgtgcagggggctctgtgtagcctgtggcggtcgcctgcacctgctgctgggtggagttggggtgactgccacttacctggggccttggcaaagcagctgcggaatctcacccaaaacaaattaaaacgtgacaactgtgaatgggtgttttagatgagggtgctctgagcccacatgca

SEQ ID NO: 81 - DNA, 599 bp, Homo sapiens
gcaagttcgaggtcagtttcccgtgtgctcattgtgtaggtgccagagacacccccgccccatcagagaggaatttacctccagagcttgttaactcagggagcaaaaactggattaaaacagccaaggcatgcaagagaaagaacatggaagtttccaaggccagtggacccactcagaatcccagaacaacctatttccatgtgagtttgggaggctgtgattaccacacacattcccactcctagctaaaatcagtagttatcagcaggtggtagctattcagtgccgtgggcagcmttcttaaaatagtcttctccatggctcctggtctgagaaggctccaatttcagaagtctctgtgttttactaataaaggctgattgtggctgggtggccacacaggcctcggttactgctcactggggtcagctgacattcagctgttggcacccccagtgcagggagcgattagcaatgcctgtcactggcaaaggcaggccgacagtggtgtgtgtgctgagtgtgagcatcagcagaaaaactcagtctaaacccctttgttcttctcttgcagagcttttgcctgcaaggctgtt

SEQ ID NO: 82 - DNA, 599 bp, Homo sapiens
caaaatggctagaaactctgctacaatttgaatataagtgataaaggcagacatcttcaccttctcctggtcttaggggaaaatcacccagtatttcaacataaaaatgttattagttttaggcttttcatagatgcctgtggtagactgaataatgctcctccacttccccacaagtgtccatgtcctaattcttggcacctatgactatgttaccctacataacaaaagggaatttacagatgtgactaacttaaggattttgagttgggaagattataatgcattatcaagttaggygtaatataatcagaggcctttttaagtaggaaggagtagtggtgtagttagagaatgaaatgtgatgatggaagaagtactaagagatcataagccaaggagtgagggaaccctctaaagcctgggaaaggtaagaaaatggacttttccctggaacctctagaaggaacacagccctgcccacctcttttaaactttgggcctccagaactgtgaatttgtgttattctaagccactaaatttttggtatgttatggtagcagaagaaatgaatacgatgccttttatcaggttgagg

SEQ ID NO: 83 - DNA, 599 bp, Homo sapiens
tctctgtccttccttttccctcatcctctttctcttcctctcaaacctcatttttcctttgaaaagtttcaagtagaatgaattcagtgttctcagccgagttccagcataaagagctcactatgcacttcgacagaattatctcctttgaagaatgcccactttaatctcctctaagacaggtttttgtggtgttagaacaaaatgacataatactcttcaagctaaaaggcttattaagtagcattcagaaaatcctgcaacttcctcgtaattggaggcttaattgggtgggtttartagcatttggttattagggctaatcaaatattccagaataattgactcaacttttttttttggggggggcaattatcatctctaatattgttgtgattaaaaagatacatcttggctgagtgaagacattgttctatgtaatgcatgtctcagtagggttggaaccaacataaccctgggccagcagcttcatggcagatttagtcatagcacatgatcatagcccaggagagcatctctttcgtctgaggtctcttgtgatgcaatgaaaagcttgattgtttccagggaacctgttt

SEQ ID NO: 84 - DNA, 599 bp, Homo sapiens
agccattaaagtattagtattactttgggggcacttgattatgcaacccagtagtaaatctcctctgggccttttttcaaaattcccaacaagagttgcagtttactaatttatttttgaatgagctgagtctgtgttcatcccagcctcctattcagctttttgcctccgtgcaaggatggtgagccatgcacaagttggcacctagcattaaagaggtgctttatatttcacttccaatcatatgcaaaggcagaaccaccagttcactcacttaacaagtgtttactgacagcttayatgtgcaggagagtgaggctgaacctagaagagatctgagtttccatctggttttgtctttctgggtgtcaatttccccatctataaaaggaggacttgggcaggaaggctcattcttgaggcaggatggttaagagcagacatttgggacccagaccacctggctttgaatcccagcttggtcagttactcgctgtgtgttgagtttgggcaagttacttaaagattcagaggcttgggctcttacatttgtcgatcacaatggtagttaccttgcagaggtgtcggaaggattaaat

SEQ ID NO: 85 - DNA, 599 bp, Homo sapiens
acgcccagctaatttttttgtatttttaatagagatggggtttcaccaccgtattggctggactggtttcgaactcctgacctcaggtgattcatccacgttggcttctcaaagggttaggattacagacctgagccactgcacccggccctggttcctatttctacctcctccctgccccacaactgaggcctcttgacatttgctgcaggctcagcccctctccaattaccatggaaaaatcctgccctaagttggaggagtcaataactccagccccactgcagactcaattgagamcttctctctcatttcatagcctgtccagcctttccagtttataactgtattccctactcactctaaggtctgagtccaagtcatggtgtctgagatcttaaaaatagaatcatcagaatgacccgccttgccagtttcagggtacaaatatagccacctttatttcaattttccactcattcaagttctcacatactttttttagtgcaaggtcccaagacaccctctttaggcatccactgatccctggattttcaaagtctaggggataacttgggcttagggcatcagtgtggttc

SEQ ID NO: 86 - DNA, 599 bp, Homo sapiens
gcttgaggccaggagttcaaaactagtctgggcaacaaagcgagaccctgtctctacaaagagttaaaaaagattagccaggcattgtggcatgcacctgtcatcccagcaactaaggaagctgacgtaggaggatcacttgagcctaggagtttgaggctgcagtgagctatgattgcacaactgtattctagcctgggcaacagagcaagatcctgtctctacctgaaaaaaaaaaaaatcctaaaaatgggcccccagtctctttttgtttgtaagtctttgctgagaaattcattkttagtctaatatgatgcttttctctagttgcctttaagattttttctttcacattaaccctggatactctgatgactatggaccttgaggatggtcatcttgcatactatctcacaggaattctctagatttcttgtatctgcatattgaactctctagccagattgggaaaattttcctgaattatatccttgaatatatttttcaagttgcttacttgtgcttctctctcagaaattccagtaagtcataaatttggtcactttatataatcccatatttattgatggctttgttca

SEQ ID NO: 87 - DNA, 599 bp, Homo sapiens
cagattctggggatgcctcaagaaggttgcaatcatgccagaaggtgaagagggagcaggcatgtcacatgccaaaagcaggagacagagaaagagtgggaggatgccacatttttaaacaactacgtctcacaagaactcactatcacagaaacaacatcaagtggatggtgccacaccatcatgagaaatctacccccacgatccagtcacctcccaccaatccccacctccaatactggggtttacgattcaacatgagatctgggtgaggaacacatccaaactatgacactatgmcacttccacagggggtaagaacactgacatggtggttcttttccatcctttgtgctgttattgtcataacttttacttctacataagttataaactctagagtacatttttcctatttttgcttagtcttttttttaagttctgggatacatgtgcagaatgtgcaggcttgttacataggtatacatgtgacatggtggtttgcacctatcaacccgttacctaggttttaagccccgcatgcattaagtatatgtcctaacactctctctcccctcacccctcaccccccccccacc

SEQ ID NO: 88 - DNA, 599 bp, Homo sapiens
aggaggtagctgggattacaggtgcctgccatcacgcctggctaattttttctatctttagtagagatggggtttcaccatgttggccaggctggtcttgaactcttgacttcaggtgatctgcccttggcctcccgaagtgctgggattacaggcgtgagccaccgcgcctggccatgttctctatcttgaaaggggtttgggtcacattagtgagtgtacttctcagactcaccaaagagtacacttgacacctctgcatttcattgtttgtgaattttaccctgaaagaaaaaaaamcaaatactgacatgaatgtgaaagactgaggagaaagtgaacagatgtctgcaacttactctgaaattcaacaaaaagggaaaatggacagagacgtaataaagcagatgaagccaactgcttttgagatggaatctcaaaatctctgtcacccaggctagagtgcagtgtgcgatctcggctcactgcaatctccaccttccaggttgaagcgattctcctgcctcagcctccccagtggctgggattacaggcgagtactgccacacccagctaatttttgtatttttagtagagac

SEQ ID NO: 89 - DNA, 599 bp, Homo sapiens
cctaacgtctagagccccagcgaacacagacaaatttcccattaatttccctatgccagagagtgacattttcctaatctatctttcactgaaaattctggcttcatgaagaattctgaattctaacttgcttcttcctgttgacccaaggctttatcttctatcccttcccggccattaaaacccaaatgctttggaccctgcagaatctcatttgtttactgccgtttaaatattcacctttgacatttttttttttttttttttgccctagggagttcctctactttctttccccayccctctgcttccaagcccagctacattttttttttttgttttttgagagggagtcttgctatgtcacccaggctggagtgcagtggcgcgatctcggctcactgcaagctccgcctcccaggttcatgccattctcctgcctcagcctcctgagcagctgggactacaggcgtccgccaccacacccggctaattttttgtatttttagtagagacagggtttcaccatgttagccaggatggtctccatctcctgaccttgtgatctgctcaccttggcctcccaaagtgctgggatt

SEQ ID NO: 90 - DNA, 599 bp, Homo sapiens
atcttttagccaccagaggagatggaattaaaaaacaaaccaaaagagcaaaagaataaggtggagcaccagcttggaatagctaacagagcatatgctttggagctacagactcaaatctaatcctagctttgccacttagcacctgggtgaccatgaacgagctatttaacctttctgaaacttatctttttcttttctaaaactatgaaaaaactacttcatatgatttttgggtacaaattagataatatatgcaaaaaatctagcatagtacctggcacaccacaggcaatgaayggaaagccgtaagatggagaaaggaccataagaaagaagagagaggttaagtttaaattaatagaataggctcatgcctataatcccagcacttcgggaggccgaggcaggtggatcacgaggtcaggagttcaagaatagcccagccaacataatgaaaccccgtctctactaaaaatataaaaatttgctgggcatggtggcatgcacctgtagtcccagctactcgggaggctgaggcaggagaattgcttgaacccgggaggcagaggttgtggtgagccaagactgcgccactg

SEQ ID NO: 91 - DNA, 599 bp, Homo sapiens
atcagcctgggcaacatagcgaaaacctgtctctataaaaaaatacaaaaaataatctgggtgtggtggaacacctcctaatcccagtttcttgggaggctgaggtgggaggatcacctgagcctgggaagtcaaggctgcaagtaggccaagattttgccactgtgcttcagcctgggcaacagagtgagaccctgtctcaaaaaaaaaaaaaaaaaaaaaaagttgcagaaaaactatgacctgcttcatggttatggtcagaaacaaaactgttttactccttctctagggcaggtrtagaaaaaaatgcctcttgcttatagaaagctgaattgtgagctgcgtttcctttatcagaaaagtctttcctgcaagttggtccttgccttctgtattcatcattttctttatcatccagatctttctcatttttactttagccaacaacactttgctaaaatactgtgttaaagatccagcttagccaggttattagagtcttgttctgctattttacagtctgcttatttttaggtttcacattcctcatgttgaagtagtgctacagatgtttatgattatcctactcagactga

SEQ ID NO: 92 - DNA, 599 bp, Homo sapiens
ggcaatggtgctaggctgggcttcattcagcttgaagacactctccaccactgacagctctgtgctggttgtgtccaggccacagaaggcacaccagtcattcaccaccatcccagcagcaatcacctcactgcctcggttcacagtccccgcctgccaagggatgggctcaatgtgagtcacggcaccaaattctctccctcctcaatgaagactctccccaaaccctgctcctccctgacctgcagccccagtcccaaactgtggcaggtaatgacccagactcccaaacactaggcraaaggatctgcctcgggagacggggttcagaggacaggaatgcttaccacaagggggacttgaagaagagaggacagctcatcctggtcttcaattgaagtcttgggatgcaccagccctccctgattgctgaagacacagtagcttcctactagcacctggtcggccactgtctgtctgaagacttccaccttgagcacatctgccagaatttcttctgtctcctgtcaatcaaagatattgtgttcagggctcagaacttacagccccagaaccaaggttctccatgcccagaagca

SEQ ID NO: 93 - DNA, 599 bp, Homo sapiens
aatggcaaaaatgacagtgttgggagggagaggagttgacagagagacctcccggaaaagatgattcctgagcagagtctcaaatgaatgtgaaatagcagaggagttcaattcagttcagccctgtgcctttccctgtggctaacaagctaaaaatggggagatgcattcctagaaaatctctattgactacctctttcttcctgcaccagttatgagcacttgatgcattccaggcactccctgtcatccatctctgtatccccagagctaacctgtaagaaataaatgccagtatgmttctataggaggcagtgctagggaatgtttaagatttcagactctgcatcaaaccaacctgggcttgtatcctcatccaactacttacttaccaatggagcaattttctaagtctctttttctgcatctataaaagggacatcctggtagtagaatttacttcattgcagtcttgtgagaattaaataaggtaatgcataaaaagaccttagcaaataagtgcttgaaaaattattatgattgttatgattatgattatatgactttacttattagtaatcatgatttctataaactaa

SEQ ID NO: 94 - DNA, 599 bp, Homo sapiens
cagatgtcgcaacctccttcccttcagctgaaagtaatgaactgggacctagatgctgggttggggagcagaggcagcaaggaccgctgggaggcaggattcactttcttataaagagaaaccgtccaaccgtacaactgtctcaagatggaatgggctttcatagaagctggaaccacatggcagaaattctaaaaccatgcatggtgtggagggaaggtgggggaacaatctctacactttagcgctgcactgacattctatgactcggaggtttccagcctggatatttagaaaaaygtgactgaattgacagaaaaggggagggcaggctatgaaggatgggtagggagcactgcgcacttgacacaagggcagctgggagccggggtctgagaaatgcaggctgggcatcaaggaagaagctggaactgggctgggtgaggaacagcgaggaggagcttctcatggggatccctatggaattgtcacaagcttaggctggcctatgtgttggccacatcccaggacaacggctgtcctgctcccgtctcttctagatgttctccccatgactgaacagatgggatcgtggggta

SEQ ID NO: 95 - DNA, 599 bp, Homo sapiens
ctcccccggtggagggtgggctgggtgccgaccagccgtggatctgacatctctgttgactctctgcagtggatctgatcacatccagcccccagtgcctgcacggcttggtggggtgggtgcatggacatgcggccagctgcggggccctgccccaccttcagaggacactgtcctccgagtactgcggcgtcatccaggtcgtgtggggctgcgaccagggccacgactacaccatggataccagctccagctgcaaggccttcttgctggacagtgcgctggcagtcaagtggccaygggacaaagagacggcgccacggctgccccagcaccgagggtggaaccctggggatgcccctcagacctcccagggtagagggacagggaccccagttggggctgagaccaagaccctgcccagcacggatgtggcccagcctccttcggacagcgacgcggtggggcccaggtcgggcttcccacctcagccaagcctgcccctttgcagggccccaggtaggaggcacctcttgctggtgctagaccaggatgtgtgctcctcagtggggcagggttttcagcagaaagtgaatgtc

SEQ ID NO: 96 - DNA, 599 bp, Homo sapiens
ggctgcatctattgtccaaagccaaacatggggaaaaccaaacaatctctcaaaggggtgtggtgagggcttaggcaaaagactggtgtaccgtggctcggatggattagtttccattctccctctctctgtttccagctttcctctcactacaagatgagggccctgtccctcttgggcgtttgccaaaggaaggtctggacacccagcagctcattcatgtcgcttcccagtttgctgttaccatgttgcagtgcagaagccataagccgcatgatccgtgagcctggccctgcaacytggctcctccacacagcagcgtttgccttcattgtcacagatgactatgagaagatgcccttggtcatctttggtctgggctctggagttctgtagagctttgagccgccagagctaagggggtgaggcttgccccaggccggtggcacacccaggactgggcctggatgactttaatggtaggacagaaggaagagaaaggagctccccagagtccagctgctgggctgtgcagcccatttgcagtgtacttccaggagccttgatggtgaatgctgtaaaagggggaatctttcttc

SEQ ID NO: 97 - DNA, 599 bp, Homo sapiens
aggtggtagggtagtgcctgagcccagtgagacacaagaagtagacataccagaaataaagccaaacatgcatggctgtttctatccagcttgctgtgaagctggtgatcgctcaataaatctccaaaaatcacaataaagaagattggtgcccattttttctggggcttacacacaggtagtaaaacagaacattcgatccttgggcttaagtaattcctcagacataaggatgcaatccttccttttcaaagcaaacggatggaacatgaaagactcctgtttagagaaccactgccrtgtaattaagcaagttttcaaatctcagcccagtgtgcaaagaaatcagtattggaaggattgttttcaggcagaaaaaacccgagggaagaaatcagaccaacattcactccagttagctggaaagccccaaaattaattattaacattagatgggcttgtgactgggctggttattgaagagataaatcatttttattgcacaaagcacagaagtatactcctcccaaaacaaaccagatgggctatgcttttcaaggaagaaagataagattcttcatgttaatgaatgatgtgca

SEQ ID NO: 98 - DNA, 599 bp, Homo sapiens
gacaagagtttcgtgtcttaattcaaaatgcccccaagtataactctgaaaacatttctagtcttgtaatcaacatcagggtaaaaatcatgtgttaatacaaaggtacaggaacaaagaatttgttcttcatggctctctgtgtctgatccaagaggcgaggccagtttcatttgagcattaaatgtcaagttctgcacgctatcatcatcaggggccgaggcttctctttgtttttaattaattgtttttaactgtgagtttatatacacttgaagcagtatacatttagaaatggtmtacttgtcgtttctttgattactacccatgagacagtattagtaattctggcctatgaaattggcaaagaaaactaccagtggtggggagggtgtgaggatggtgggaacatgaactgttataacctataatcggatgtatcatcaaattgtcactgggcctctgacccactccacttctcagaacttaacaaaaggggtgaccgaagatacacccagatagccctgcctgtaccacagaaaagtgggcacggccccccagcaccatcagagaaatctgacagagtaaaccaggacaca

SEQ ID NO: 99 - DNA, 599 bp, Homo sapiens
ggcctggactgcttcacactgacacgcttgtaggatcaagggctgaaagtaggaggctctgggccttggacttggaagaaagacttggatttgaattcccaactttggaacctcccactggagggtggaacccctcacaggagggcgcaaccccccaccggaggatggaaccctgcaggaatagcatcacccttgggtggcagtttggaaaatgctacctgtctgtcacggtgcttttagctgaaagttacagaaaagctcactaaacgcagctttaacacagggatttaatgtctcacraatgaagggtcagaggtggagcttccaggttggcttgttagcacaacagtgacatcaaagactttgcgtgtctcttcttgaccactctcagtgtccacagcccccgatgagtgtacagccccccacacgcaggcaggactgcatctactgcaaaggcattgctccttgcttcatattagcaaggaaacctttcccaggaaggcttcgcctgcccagcctctgcaggactcattcaatggtgcaaattcaccaggggctggagggagctgtgaagcatgggagtgggcaggacctctgcc

SEQ ID NO: 100 - DNA, 599 bp, Homo sapiens
ttagtataatatgatctcatttttatgaacaaagaaacaaacgcacgcatattataaacaaatgacagataaaattctggaaggatatataccaaaatgctaaaaagtggttatctttgggtggtatgtctgcatgtctgtctcttctaattttatcaaacgagcatgtgtcacttttcgagagagaggagagaagacgcgactgtggaagaagagctcacttcaggcagaagaacaaggaatccctcgtcctacaggtcaggaggctgtcaaagagcgcagggacaggaaggccagcaycaggtgcaggaggacccacatccacctctgggagcgcagacctggactcacccaggtgcacggccagggcagccaaccccagcacatgtggggcactcaggctcgggccctgcaacgagaatgagggtggcagagcagactgccctcttccaagctggaattttccagggcactgaagacgaattgagaagtagcagcctggccctcagagtgggcccccaagggtggcccccatgaaggagagcctcacctggtgacggagcagctggcagagccgggtgagcactgcagggagcaca

SEQ ID NO: 101 - DNA, 599 bp, Homo sapiens
ttgggaggctgaggcaggagaatcgcttgaacctgggaggcagaggttgccgtgagccaagatcacgccattgcactccagcctgggcaacaagagcgaaactccgtctttaaaaaaaaaaaaatcctccttcccttccctcttaccttgctgcctaggtgtccattcttcatcggaatcttctgtgtcatccacctggagtttgatggcctgccttcggtgacacatgaaagctggtcgagtggctctgagacctgaaaagaagcaaaaaattttgctctgaaacggaagagctgggakgaggaacaaagggcagtggcaatggaaagcaccacaaagccagtgctgctcagtctgatgagctgggagagtcttgaacaaggtcaagatgtgacctctgcatggtcagtatccacacacaaccctgcactgacgcaaaagaacaatatttccctccagatttgtctccgtgcaaaagggaattgtgggcagatgccgccacctgataattaaaataatatagggaccaaagacctgttttatatcgcccaggctggtctgtgcccagcacttcctgttacctacagtaatcagtgcat

SEQ ID NO: 102 - DNA, 599 bp, Homo sapiens
gcattgggtcgtggggagaccaggtccgtgttgaaccctcccccaatggagccacatgggacgcgctgacttcccccagcatcaagctgtgaccacgcctgcctgatgttgaccaggaagctcaggagagacttggttcccagggtttttgctgggggtgcacaaagcgtttgggcccagggagctgttttctcaaggaatggtgggaacgctcccagaatcacagtgcccagacactgcccttgcgagcaggcctttctgaggacagcagtcaggcctgctgtgttcaacacttctctrcgggccagaagcatttcctaaatattgttttgtctttagtacccagtagtaattctaattagatgatgactgttttgatatttggaggggaccgattggagaggagggaagtggagcactcagcctttctcctcccttcccctcggctgggcattccatttcctgtgcagcccctggcttgccctcctccactccagcaccatgtggtgtcagaggctgcaggaacccataataagtcaggataaggagggcccacgccctggacttcgctgtaccccctgagccaggctgtggggttc

SEQ ID NO: 103 - DNA, 599 bp, Homo sapiens
ccacctacgtcaccctcccaaagtgctgggattacaggagttagcctctgcacccagccacttttgtgaacttagaagtgactcatggcaaacccatcagttgcatagggttcgcagttatggggtgaggtggtcgatttctcaaagtctgcttagcaggaagctacctttgagcgcctccaccccttctgggccgctgtggaaagggctgtttccaagtccagtgtcctggtgcccagagcacatggcttgtggggattgctggcttgtgagcctggctggccctttgtgttcggaaaygggattggaaatttggggattttagatttaagacattttcagaagaactcaaatgtgggacatgggggtccatttggtatctttaggcaggaggtcgtgttgggtctgtcactgcccaggcccagggctttgctgaagattctctcttaattcctagagacgtcttgagtgaagatgaaaatgacaagaagcaaaatgcccagtcttcggacgagtcctttgagccttacccagaaaggaagtaagtgggcagcccggggtctgctggaggcatccgttgggcgacctagacacccgcc

SEQ ID NO: 104 - DNA, 599 bp, Homo sapiens
atggggaaaaatgttttctggatacagcaggctgattttgtgaggatgtggaaagcaaacatctttatagagcctttccctgtccctgcacgttgcagggcccgccctctgagcgggtgtccctgaccaccagcccgctcctggccctgaagggcaggcccaaggttcacactttgcggaggggagaccggcaaggcatgctgcatgaagtggagcagctttaggggcagacataggatgcgtgagtgtgctgccgatggctgtgacttcctggggcagagcttgttttcttttgttttkttttttttggctcattcttccatgggggtggactttctcagcccatttatgaacacagaggaccccttcccagtcgagagagctctgctcaagatctgctaggagtcatttgcatctcagtgacatttcagatccatgcagtttgttttctagggagagattgaatacccactctaattttgatgggcacactctccatgcgagtcaggtgtttcctcaaagggcttcagaacacctcacatctatcgtgcttattttccataaagatgttggatgcaatctgatgaggtgcctcagtg

SEQ ID NO: 105 - DNA, 599 bp, Homo sapiens
cagctttgagggatttagtgttttcactccagtgcattccattctaaggaaacgcctgaccacggctcatggctaggaggccccgcagtgccagatgctgccctggagttcaagtggaacatttgctgtctgaacctttccaaactccccccacccatgacctcatggcccaccagggggcgactttttcctgggtcgcctggcagagagccggagggggtgctcagggggcagctgctgcaggcccaggcggactcagccgcgctaggtcggctccgtcgcacccgtctgtgcacactracctttaggggaacttgccttgcttcctaagccgcgactcagccggggctgtgtaaaccctccctgcctgttcccaggctagtctggctcctcgcctccttcctcccgaccgcggagcacgtgcactttacctgcgcacttgcagatctttctccaggagtgagtttaaggtccgcacctccgctctggatggtgagcggagcacagccttcgaagtaagaactcagagggagctcggcgctcccgcagcgggtcccccacccaggcacgcccccggcccggccccgcccacagcactc

SEQ ID NO: 106 - DNA, 599 bp, Homo sapiens
cgggggggcggggcagagggaggagggcggagggcggggggcggggggcggagggcagaggagtgaaaacaatgagccgcccccaccgctctgaatccagcaggaaatcctcagagtgaaggtgaaggtgaaataaaagcgctctgaagtaaatgaaactgagaacatttgccgtcctaagagccgcatgacaaaaacatgctaaagctgcgtaatctgcacaagataaggaactcactgacgggcagggtggcgacagcccatgtgcgaccgtgcaggtgaacaaggcgcctgtcctcycctcgtcatctcttttaaagggcaaatctgcaggcgtgtgtgtccagcatcatgagcttcgtggggtctgagtcatagaacgtgtgaggacaggagcgtgaggcgtgggcgtggggagggaaccgcactgctgcaaggggcttgggtttctcatcaaacggcgccacatcttcagatgtctctggaagccgaattgagactggatttgggtgtgtgaagatgtttaggaccagcacgtgtgaagggaagcaggcagggctgacgtgtggtgcaggccccacggggccgtgtgaccccac

SEQ ID NO: 107 - DNA, 599 bp, Homo sapiens
aagatgctgagggacgaactcgacttgcggagaaagactgagctccacgaagtggaggagaggaagaatggccagatccacacgctgatgcagcgccacgaggaggccttcaccgacattaagaactactacaacgacatcaccctcaacaacctggccctcatcaactccctcaaggtgctgtgcgtgggctgggctggggaggcacactctgcctgtcctagaatgtgggctgcagagtcctggggatccagaccccagctctccaccccaacacacccagtactttttataagattrtttgacaatgcccctctcatatccttaactgaaattcctaaataacgtaacctatctctacctattgttaaaagcagtcaggatattgccctcaccgtagtgcagagggggaagtaatgaagtgtgttctggtttgtaaaagctcagatggaaccgcagtgagggccgtgatgtggctggagtggataggtgtcatagtcactgcctgcgacaggtggataggtgtcatcgtcactgcatgctggcagtgacaggattttctacagtagacaacggcccttggtggaatcttgaacaaa

SEQ ID NO: 108 - DNA, 599 bp, Homo sapiens
tgcttaaaaagtaattgctgcccatattctctcatgtttgagttctagaagtaaactttaaaatattacttaatacctatatgttactttgaaactaacatgactacaaacagcaccccaactcccaaattgaaatgcctaaggaacctgcagttaatcaatctgtgaccttttggatacactgtgaccttgggatgtcatctgatcctttgttctctatcttctgcttttgatgaaggcagagcggccatgtgtctgcaaagttatttattagagacaagaggaatgcatatcaggatrtattcataaacttttttgatctctttggaagatgacacaaatagaaatatcattaaatttgcatttataatcaaaatccaaaaggatttggaagatgattactttataagcaaatgtaaaaagaaaagacaactaagaaatgcttgcaaaagtaaagcattccctgatgaagtactctgaggttgaatgtcattcatattttaattaaagaaccaaacatgtcttctgaaattcaactgcctaagctaggttttacttcatgaaaagattataggacacttggcaattagaggttgtgg

SEQ ID NO: 109 - DNA, 599 bp, Homo sapiens
ccctttgttatctggccaccgcttctctctccagctgtgtctctcctccattccaccttcatttttctcttcagtaatattaaatatcttttggattttagatcacaatgggatttctcgtgctttgcagtcttttcccctttcactctctccttggataatgctttttatctctcttccttgcattgtaagcccctcgcctaccttccagttgtaattcaggtggggtcttttgaaggtcttttatgaatacctcttctcttaccactctgctgccagaagcaacctacccctggcacmtttagcacttggcacaactttctatcatgtccctattcacacactactgtaatttctgatttctgtctcctctactatatcttgttctctatttgattgtaaactctttgagggtaaggaccttgcttttcttaaaaaaatattctttttatagcatctaacatctagcaatagtgcctggcatttatatatatagcactcaggaaacatttgttagaaggaaggaaggaaaaagagaagaaaggaaaaaggaaggaattagaatgactcattatagccatccatcatcaattgatgac

SEQ ID NO: 110 - DNA, 599 bp, Homo sapiens
agaaatattttggtttccttcagtccccccttgaagctttactttcagaaagtttcacatgttaaaagctaagttggtagttttggagagatttgggttagaagttttaagataagagatttggcaaaggaggaggagaacatagtttggaacaagcagaaaggaaccaatttgggtacattgttccatctcttattgtatcagtctttcagtcctgaaaataggtcagttccgttaaatcattgcatcccatttcagaggtggtgtaagtgggcttccataagagttacgtcttccatrtggtgtaggcacacaggtactttatcagagacatttgtatggaaataaaaagaaaacaaaggtctatgtttcaagcagtctataagctagtttctggagagcaaccagctgagatttctagatttgggttcaaataattttaagttggagtggacaggcagtggcaatctgacagatttttctggattatagtttgcatcagacgttccagtgaagtttttgtccataaataaattgtgatgacttctccaaagtttatatcaagttgttcagctttcatttgcagggcttcaggaaag

SEQ ID NO: 111 - DNA, 599 bp, Homo sapiens
ttccagtatgctttctttgtttggagattattttgttttcatccaaatttctgtgtatttgctccaaaatcggctttgcattgactgtgtagacctggctttggattccatagacaacatttatacattcacttactcattctccatccagccacacttggggaatatctgttatgtatgaagccctgtgctaggctttgggaatagaagaatgagacatggccccttccgtgaaataatctagcagagagaaagacccataaataagcaaaaggcaatgtactcaggctacagaagagrtgtaggtgcccagaaaaaggaacagctatctctgcttggggacacagagaggaccttccaggcttgataacaactaaaggggtcttgaaggatcaataggagtttgctgatggataagtaggggaaggcatttaagcagagaaaaggcatttgctgtggcaagaggcttgaaaagacctagcacctgctacagcccaggggagggaaggagcgtggaatgtaagttcacaagcagaccaggcccatactctaaaagggtctcccgaggcccaccaaagggcagtgtaggatggccttag

SEQ ID NO: 112 - DNA, 599 bp, Homo sapiens
caggcctgagtgagagaaaaagctccatcagaaatagagccaaaagttcatatcccaaatacacagtaacactcacatgaatgactaaaaagaaaaaaagacaaccagatagaaaaatgcgcacaaaacttgaattggcacttcacaaaagagaatttccaactgattaatgcacacaggaaaaggtatcaaacctcactaataatctagaaaatgctaattgaaaccacaatgagataccagtgtacacccaccagaatgggtcaaacagggagtgctagcaatgctaggtcttggcayggaagtggaacactgagaattctcactttgcagtggagatgagtagcagcacaatcaccgtggaaaactggcattctgtattaaaggcatcctctacaaaccaagagtcccaccaagaaacagaaagaatgattaaagaaacagatgtaagaatgttaacgtattggcaggagcgctggctcatgcctcttatgtcagcactgtgggaggatcacttgagcctaggagtttgagaccagcctgggcaacagagggagaccgtgtctctacaaaaaatttaaaaattagccaggcatggt

SEQ ID NO: 113 - DNA, 599 bp, Homo sapiens
cctacaagtggtactagctacaaaacacatggagaacacggttcttgcacacacaccttatgagatgtcggagagctacacagcggaggcatacagggcgtagccagacggtctaaatgggccatgacaacaaccgccgtttgtcgcagatcaatggcaagctcgttgtcttgtggaagggtgaggtacctcaggaaactctcattggggctcagagggccagacaaaagctagaaaggaaaagtaaacaaaaattcagaaatggtgggaaaaattaaagttaacacaattaacctttayccacgctttatattttggtattgactcatttgacccatcaaatgacaatgtcaatgatacagttatactatacatctatatatttatgcagcatataaactgactgtgtaaatgtctaaatttgctgtacacgcatacacaacattgactgcgcatgtatacatttatgataccacaggtaagatggatccacacagattactaacatgaccaaaccaccctacaggcctaggacccctggagaaaggcagaaccacctctgtgggaacccagcacagcatctcaagctggccttgaaa

SEQ ID NO: 114 - DNA, 599 bp, Homo sapiens
gtgtgcccatctcagacgtgggcctgagggtgcagccctggccctgtgctggccatttctaggagcggtgccctgaggtcccagctgtgatagccccacgctctgcaggaagagatcatgggggcggggagttggtgctgcggcctcgttcctctctgcagtgagtgaacgatgtttgtggtcagcaggagcctgtggggagcacaggctggtcctcctggtgtcccacccacccctttttccatgggggatctgcactcatctccagggaagatggttgggagataaccccagtctgcyctaggtccccaccctccacagccagggtggtccgtggtgagcttcagccatcgagatgcgggagtctgctagagtcttcagggtcttttctctgaaaatgacaggctagcaaggagacctgggtcccctgcctcttccattccagatgccttgagtccacccaaataggggatgtgatgtttggagctgcagcagccgccctacggttgggagtcagagaagagccggtgttccagggacaatgcagcagaggctgagcccaggcctgctgtcctgagaggtggctggatcactgacac

SEQ ID NO: 115 - DNA, 599 bp, Homo sapiens
cagggttttgtcatgttgcccaggctggtctcgaactcctgggctcaagtgatcccccacctttccagagtgctgggattacgggcgtgagccaccgcacccagcctgctggttaagatcaccctgcaactcacgctgggcagtgtctaaaatggaagataactgggctgcctggcaggcaacagcggtagtcagatcaccacaaggctttttttacacatgaagcatgttgagattagaaataaatgaaccgaggtgcgtggcacacaaaatggtctgtacgattccatgtgtcaaaaycattaaaacttgagggaaactaaaggaactcaaacttgggagaaggcaacctacagctggtttggtgatgtgctggtcgccgtcctggctgtgggtggggtcctccctcacatggcattccagacactgttccctatttgactttgaaccctttcctcagtaattcacacggtcagtacacgcatggaggctgcaagagctgctgttagcgccacaggcactgacagatcggacggacacgtgcacacggggcacctaccatctcagagttgaccaagtggaagaactgctcgcatctg

SEQ ID NO: 116 - DNA, 599 bp, Homo sapiens
tgcagcctcaacctcttgggctcaagcgatcctcccacttcagcctcctgagtagctgggaccaccactcctggctaattaaaatttttttggtagagacagggtcttgccatgttgcccaggctggtctcgaactcctgggttcaagcgatcatacaccctggcctcccaccccctcctctctggcagccccctaatatcaaggaaacctggggccagagtagttctgcagagaatctctggggacagcaccctcagaggcaggtggggcaggtggttctgtgttaataatgacagctrggtgtggtggtgcacacctgtgtggcagctactcgggaagctgaggcaggaggtccgcctgaaacccaggaagtcaagaatgcagtgagctgtgatcgtgccatccagcttgagcaacagagcgaaaccttgccttaaaaaaaaaaaaaaaaaaaaagacagtggcttatggttttctagtacagtcccagcattctgtgtttattgactaactccattctcacctcaccctaactcccagctacatggggaagactctgagctttgtacttgagaggaggaagctgaggcacagagca

SEQ ID NO: 117 - DNA, 599 bp, Homo sapiens
taggaggaaacggctctgtatgcaggcagttgactgaggcgaagtttggggaattggtagtaggaccaagtgagagaagggtctctccatggggtgtagctgtgtctgttggcattttggccaggagcggcctgctcaggaacaggtacaggtgttcccggaaggacaggagtgccaccctctgtgccctttgccctgggctggcatacatgaggcctgtgggcagacacacacctcacgctgcccagggagtgtcccaaagcagtgtggcgggtacctggggccgaagccagggtgacyggttctgccttggttgtgggggctgtgccaaagggtaggtgcttctctagagttgggacttcgtgacagcagaggctgcgtgccaccgagatcgtgcgtgacggaggcagcaggtgccgcgccttcttcccaagttctcagcacagagcgcttcccaaacaggccctcaccacagtgttggcaccttccccagttcacaaagtacatacccatgtctgtttccctggacttgccaggcagcagctcatccaccctcccatccacctgtccaccacgtatccattcacccaagccatgca

SEQ ID NO: 118 - DNA, 599 bp, Homo sapiens
tgcccaccccctcccaccctgagagcgtgggcagggtcccctggccttaccttggaggccgcatcgggtggggccggggaaccagggcagcagcagcaggaagagcaggtagaccagcgagagtccgctgaagcggagcaggcaggctgcggggagatgggcgttaactaggtcgccaacagaggatggccagccccaccccagaggacagcgcacccacggcccagctcgtcacaccgccgaggccgtgtgccctgtgagtgctgccctgagccagagccgtggaagccgcctcgcgcytccgtacatatctcacacacgccacgacacgggcaccagcctcccagcggggcgctggggttttgacccatctttctaccttgctttttttcctttaaaaatgctattcttgggctgggtgcagtggttcacacctgtcatcccagcactttgggaggccaaggcgggcggatcacttgaggtcaggagttcaagaccagcctggccaacgtggtgaaaccctgtctccaccgaaaatacaaaaattagccgggcatggtggcgggcacctgtaatcccagctactcgggaggctgagg

SEQ ID NO: 119 - DNA, 599 bp, Homo sapiens
tgaaagtttttctgaaagtgttgggattggttaaggtctttatttgtattacgtatctcccgaagtcctctgtggccagctgcatctgtctgaatggtgcgtgaaggctctcagaccttacacaccattttgtaagttatgttttacatgccccgtttttgagactgatctcgatgcaggtggatctccttgagatcctgatagcctgttacaggaatgaagtaaaggtcagtttttttttgtattgattttcacagctttgaggaacatgcataagaaatgtagctgaagtagaggggrcgtgagagaagggccaggccggcaggccaaccctcctccaatggaaattcccgtgttgcttcaaactgagacagatgggacttaacaggcaatggggtccacttccccctcttcagcatcccccgtaccccactttctgctgaaagaactgccagcaggtaggaccccagaggcccccaaatgaaagcttgaatttcccctactggctctgcgttttgctgagatctgtaggaaaggatgcttcacaaactgaggtagataatgctatgctgtcgttggtatacatcatgaatttttat

SEQ ID NO: 120 - DNA, 599 bp, Homo sapiens
cgccaagaaccggaacctgcactcacccatgtactgcttcatctgctgcctggccttgtcggacctgctggtgagcgggagcaacgtgctggagacggccgtcatcctcctgctggaggccggtgcactggtggcccgggctgcggtgctgcagcagctggacaatgtcattgacgtgatcacctgcagctccatgctgtccagcctctgcttcctgggcgccatcgccgtggaccgctacatctccatcttctacgcactgcgctaccacagcatcgtgaccctgccgcgggcgcggcragccgttgcggccatctgggtggccagtgtcgtcttcagcacgctcttcatcgcctactacgaccacgtggccgtcctgctgtgcctcgtggtcttcttcctggctatgctggtgctcatggccgtgctgtacgtccacatgctggcccgggcctgccagcacgcccagggcatcgcccggctccacaagaggcagcgcccggtccaccagggctttggccttaaaggcgctgtcaccctcaccatcctgctgggcattttcttcctctgctggggccccttcttcctgcatctcacac

SEQ ID NO: 121 - DNA, 599 bp, Homo sapiens
agttccctgagcccacagctttggcgccgtggcttctgcttcagggagtgtgccagtggctcgtgtgggggactcccaccaccctggactctcggtgaccctgacagtccacgaagctgggacggttgccatccgccaggaggtgaagtaacaaggaatgcgtggcctgggggcggtcagggccaggtgctggccggcccctcggctctgcgtttaccttcccgcggctgtgccttgccgctcagcacagggcgtgagtcagccccagtggcctgaggcgtgtttcagtttcctgctgaytcaagggtcgtgagtttaaaatagactttgcctgataacttggaaatgagggagatttaggctgcacttaaaatgagtctaggcagggacagccaagtcaccttccagggaagagtctcccccgggagtgagacccggtgccttctgttgtgtggtcggctgtgcagcatcgtgatgagaaggcacaggggctgcggaactgtctaaagaggggactcccagcttcaaggactgttttatgtgacagccctgccagggaggcctggggacatcatcacagcccccaccctcagacaaca

SEQ ID NO: 122 - DNA, 599 bp, Homo sapiens
atcatgccacgtttccctgccctcccctccattcactcccctgctcctatcccagcccatcctcccctcctggccactgggacccacctgggggtccccatgagcaccttcccatcctgcctggcgccctcagcaccagacacagctaccttggtgctcccagctgccctgtaggtccccgcactgcccgatccgcctgtgctgggcaccctgcccccacctcctctctctgcctcacacctggattcagtcccttgagtatctcgtttacactttggcgcctgtcaatctcaaatccaygctctccactgcagtctcgcatcctcatcagtatcttgttttagaaacgcagagctgtcagctccttgcacaaaaccaaaaccctccgacaactgcccacagctctgcccatggcctccggggtccacggaacaggcccagccagtcagtccagccccttcccagacctcacaggccacggccctcaagctgccagtgtctgcccagccggccggctcagcccccgcaacctgcagaacgccacctctgccagggctgccgggctcctctccggcatggcacccaccaccacacttggg

SEQ ID NO: 123 - DNA, 599 bp, Homo sapiens
ttgcacccctgtaaattatgacaaccacaacaagagtagatttgctgaatgaaagaatttgaattttctgaggtagtttaaatttcaagtctcttctaccttatcaattagtatatttatatttacaagatcgcacagattgatcacttagtctgggaattttaggcatctttttataccataaactggggaaaaacatctcccttaaaagaattttaaaaagacatttgtaaatgtacctagtgcatattctcaagtttccttatacatggatatcgaactaatgattgtagaaacacrtttcaagtacatattctcaaagaactgtgttaaatgtagcctattcttttccaaatgttgttttctcctggtatactatttgagattctgaagataaaacatacaaataagtagatggagtcccaattttgctatttatcagctgtgtgaaagtatcagtgaggacagttggggtgatgttgcaataacagagaagctgaaaaatcacagtggtctcactcccacaacaagactaatcataagtcagctgcaaccctatttcttgttgccatcactctgggaatcaggctagtgaagaa

SEQ ID NO: 124 - DNA, 599 bp, Homo sapiens
tttatagtctcctcgtttcattcaggtcagtaaaggatcagcaaagagaagcaaacattcagcatgccatggctgggaccctaagcaccacacaggccaggctagaaagtgcctcctagacccagcatgcttttgctcacagatgcgtaagctaagggcgtgtgagccgcaaagtcgggcctgggcttcagccctgctactgcgactctgccgggacaaagggaggcactgatccccacaggccatcccagacactgatacatttttgacttgccagcatcttcgctaccttctctttcrttttcctataatcccgtttcctttcacagcatgttccctaggtgaggaacagcaagtacctgtaggtctggaacgagggcccagggccacgaaaaccagccatgggatggaacaggagggagagaggaggggatggacatggcttctgtgtctgggaccacacgcttcatgttttttcttcccttggcatttctgtcttggcacaagggaagtttcaaaacagcaggagcgaagtgttttccagctatggccaggcacccccagcactgaacacaagaatgctaaacaaagtcctatgg

SEQ ID NO: 125 - DNA, 599 bp, Homo sapiens
gtcactggtgctcaggactcaaactggctgggaatgtgagaaaagctccttttccctcctggcctcctacttgagactaaagcaaaatgctgagggttgaggaagggagagcgagtgctcgctgagaaaggaagaaatgagtgtttagacttcagccactccctgataaaggggaacgatcttctgcctcctgggaaaaactttgatgagacagcaaacttgagaaaagtttgtgggaaggagcagtgagaaccgcaggttacgaaggggccaggtgggcaggactgaaggataactccmtggtaaccggcgtggatcgatgactatggggccaaagagtgggcatgcctgggtgtgtctgagcagaaactgcattgaggacaagatcccctgcccaccagcacaggctctggctctgtgggagctatgccaaacttagccccagttcaagggaaggagagggaggagagggaggagagggaggagagggatccaaagtgaactgaacttgaatttgcatcaccctagtggaatatgagcttaaaattaacaattatattgttaaagaaaatatgaactacatgtcttaaaaactttgg

SEQ ID NO: 126 - DNA, 599 bp, Homo sapiens
agagctccaaaatgctgttcaaaagccaacacggtgaaacctcatctctactaaaaatacaaaaatcggctggatgtggtggcacatgcctgtagtcccagatactcagggggctgaggcaggagaatcgcttgaacctgggaggcgaaggttgcagggagcagagattgcaccactgcactccagcctgggcaacaaagcgacgctccgtctcaagaaacaacaacaacaaaacaacaacaacaacaaagaacatcttcccattagcactggagcagcctccttggagcatagaatgcrgtgcgggcgggctctgcactattcaccttcccctgcacgcccttgtctcctgagtgccgtggtgggatctttgtggctttgttccaagttgtgccactgatcggctgggtgcctggggcacctcagttttcatcatctgtgaagtgggggtgcagcagcacatggctcacaaagctggggtgggcatcgcgtgaattacacaggtggatgcggacagcaatgtgggccccgggtcccagcttactaaggggagtccccttgcagcagatgagtcctgaggcctttcgtgcttttgtcca

SEQ ID NO: 127 - DNA, 599 bp, Homo sapiens
tataactgaggaaatggaggcaccccgtgttgggtcccacagctcaccagctcagccagtgttccctgagcctggcgctggcgggccctgggcaccgcgatgggtgggggccgggcctgtcctctcgggctgtttggctcaggagtcacacaggccctaagcatggggatgcggttccaggcgccggcaggtgccacagaggaatccagtgggtgtgtgaggacttctcactgatccccagaggcagggaggtggcggctgggaccccagggagcaaggtggagcccagcagaagggagyacgaggaatttccaagtaagggaccactggctcacctggagggaaaggcaggacccagagccccaggaggccgctgcaggctcagatgagaacggagggctgcagccagcacattctggatggactttaaggtagagttaatgaagctggtcaatagattcggtgtgggggcgagagggaaagacaagaggcaagaaggaccccaagagtccaggggaagctgaggctggagacgggaagcctgggcgtggccagcgcggacatggccccaatcccctggcctgcctgagaccctgtga

SEQ ID NO: 128 - DNA, 599 bp, Homo sapiens
tttgaaattttttcaaaatacatatttgggaaagaaaaagagcagagagcacttgttaatttggttaggggagaatctgttgcaaaagcttctgctgtgtttccatttgccaccagttttctctgaaaattgatgtatcagatctttgatattatcgtttaagttgaaaggtcatggttattataatagttccatcacaggagatgagtagtataaactgtgtaatactacagatgaattaacaggcagtcgcagtcgctactgtgggccttagcagtcttagaattttaacaggcatcygccctctctacacattacctccgtaatccatttatctgatggctaaaccactctcaaaaccagcagggagaggttggaaagtgatctcagaattagctggaaaattttttcacgtacacatgtccagaaactactggccatcaaactgttccagcctccagtttttgagacctcctcgggcctggacagacatgtgtattttgaaaaaggtcgtgagtctaaactcccgcttcactctcccactgagaatgactttgagagcttccttccttgaggccatagctctaagagggatagca

SEQ ID NO: 129 - DNA, 599 bp, Homo sapiens
tttagcacagtgttcctctcttactcttcatttctttcttgtggtttatttcatcaatggaggaccttgatgtaattgtttccctgtaatttgtagataatatctctctcactaaatatagaacactgcctctatctgtgcattttacagggaagtcgtaccttgcatggaaattaaacaagaaattagaattactaataatgacttaacggttgaatttaagatgactaaatgaagacaataacatcattggcataaacatgcagggtaacaattgtacccattgcaacatttcccttygggttccagcctcacagcttccagagcaggcaccctctgtttatacagtactctgcccattttttcagctgccttggtggaagccagtctctagaaaaagcaaggtggttgcctccctgtgtgctctgctcagttagcgcctggagaattctgtttgatgttcaacattttaggagcaggacacaggagtgaaggcggcgcttgcttctctcctgcagtgccacgactcactcaatggagtctgctcatcttgggatgaaggacaggaccggagacggtcgtcactcccacagttatac

SEQ ID NO: 130 - DNA, 599 bp, Homo sapiens
tgtgggaaatccttcactgtttcttcaagcctgactgagcacgcgagaatccataccggagagaaaccctacgaatgtaagcagtgtggcaaagccttcacagggcgctcaggcctcactaaacacatgcggacacacaccggggagaagccctatgaatgtaaggactgtgggaaagcctacaatagggtttatctactgaatgagcatgtgaaaactcacacagaggagaagccctttatatgtacggtatgcaggaaatccttcagaaattcctcgtgcctgaataagcacattcakattcacactggaataaaaccttatgaatgtaaggactgtgggaaaacattcactgtttcttcgagcctaaccgagcacatacgaactcacactggagagaaaccttatgaatgtaaagtatgcggaaaggccttcaccacatcctcacaccttatcgtgcacataagaacccacaccggtgagaaaccctacatatgtaaggagtgtgggaaagcctttgcttcctcctcacaccttatcgaacacagaaggactcacacaggagagaaaccttacatatgtaacgagtgtgggaaagc

SEQ ID NO: 131 - DNA, 599 bp, Homo sapiens
ctaagggggtccaacctggccggcccgagagggtagggcttcttgggagaaagctgaaggtcatggaggagaaatgggggcagggaagaccgtcaggctgaaaccccacatcgggcagctcagaggtgggggcagggaagaccaccaagctgagtccccccatcaggcagctcaggggagggggctccatgctgggcccctgagccgcctcctgctgtcacagcatgaggtttatcattcagcacccaggcaagctggccctgcaggcaggggtccggcctccaaccgccttccggctgmcccaccatgtccagggacctgcttcagccctccagtcgtgcttcctttcttccctctgctcactcggctcctcctggaagttccttctcgctagcccccagggggtttggatgctggaccctggggaaggggccttggggtctgtagcagggcatctgagggtcctgggacacccagaaaccccaggaaggctgtgggtgactcagcaaacccacaagccggggggtctctcaggcatgtgctgcagggggggtatgatgtggggtctgctctgagtgagggtctcggcccttgtctgc

SEQ ID NO: 132 - DNA, 599 bp, Homo sapiens
agagatcatgccactgcactccagcctgggcgacagaacgagactcttgtttcaaaaaaaaaaaaaaaagcatcttttcttatttccacattcttgagacagcaacacatctgtagctgatggtgtcagcgttgatgagatccagtcctgtaagcatttgaagccaccaggtgtctagtctggtccttccccatctccctttggcctggtctttattctgtaaagtctggtgccgtggtctgtgttggggctttggggaaggaaaatttggaagatgcagcttctgccatcaggacacgrattttttggccagaagaatgtgataccagggacaagacctgagcccccaggaggggcacgatggatcttggcatgaagagcgtgcagctgctataaggcatgctcgtgggtcctcaaatgactgagcctactgtgtaatacatgcagcatacagatatgtgttcgttgaccatatcagtaaagttctggtcaacagtaggctattagcagttaagtttttggggagtgaaaagttctatgtggatttctgactttgagggaggtgggcacccctaacacttaacgttgttcaagagtca

SEQ ID NO: 133 - DNA, 599 bp, Homo sapiens
aaagtcatggccctcgctccaccgtggttccctccaacaccccgggagagcctgggtgtccctcttgtcacctcccagacggctgcacgtgcctggggtccttcatgatgatctgggcgtgggatccttgtgcctcccccactgggctgttccctccagacctgtgttctcggcgcctgccttcaccctcaacttttcagggctgtcaggcgcctttcccgcagccttcccgcacgtatcctttacaagccggtccttatgtaactctggagccatctcaccccctgaatcagacacacyattttccgtggtcttttcccagctgtcctgagcatgacctgcagtttgcctcatcagcctgtcgatggtggctcagctgcacctttgctgagaagcaccatcggcccagcagagtgacacctgcgggcactgctgtctgtgctgtagcggtctgtgcaccctgcagggacacgagccttcagcccctgtatttctcagcctgcctgcctccctccctctgccagaagggaaggtggaatggggtggaatgcgtgtacctgccagtagctgtgcacccttcccagaagatacatcactga

SEQ ID NO: 134 - DNA, 599 bp, Homo sapiens
agcatggtttacatcaggaagagatcgaaatcagacccatatcactaatcggcaactataaagctgacatgttgccaaatccaatcacagttcccactgctttctacactgtacagtggctgacactactgagttttaaacattttttccagatccctttgagaaaaattgttaccctcactatccaagctgaaatctacttgcagagaccaggataactacaggctgcagtttgtatctgaagaataaagctaggtgttttttcagcatttagacatttctggagccatgcaaatggtrcatgcagtctgagacacgtgctttctcttccaacatcagctgcaagttctaaatgagacccaagtcccgcagtccttaaaatagagtcctgctccatgcaagttcttctttatagatgattaattcagtgccagtttcacagtaaaacattctgttccaatttctgaatcatccatataaagtcatgggtagcaagaacagataaagatgtggtctgtcactccagacagaatatgaatttgtttaaagctgcttcatttatgaagcaaacatgaactgttaccagcctagaaaggaag

SEQ ID NO: 135 - DNA, 599 bp, Homo sapiens
ggaagtgccggttcccgcccaccttgcaggggagcctgcccctcctgcctcgcccagcgctgtgctgggccagccggcgagccctctgttctggcgttgcaggtgttcctggtgctggatgcagatgtgctgcctgctgagcgtgatgacttcatcctgggggtaagttctgagctgtccacctgtcagggccagggtgaggctgggcagtgctggcagccctggggcccaatgctgggtgcctcttctgcccctttcagattctcaactgcgtcttcattgtgtactacctgttggagwtgctgctcaaggtctttgccctgggcctgcgagggtacctgtcctaccccagcaacgtgtttgacgggctcctcaccgttgtcctgctggtaaagtaggcgcatccgaggccggcctctactgggcgggtgggtgagcgccacctgggctctgtgctggcccatctcaggcctcccctgaggactagaggctgtaggaaggtgggcttctgctctcagtggtgagggctggcttccctgctggccgagttgctcagtgggcagccggtgaggtctttaggaggctgggtttctactgca

SEQ ID NO: 136 - DNA, 599 bp, Homo sapiens
acctgttttcctgcctctgagtctttacacatgctgctccctctgcccatcatagtctaccaggcaaacagctcttcttttaggactcagctctagcaccatctgtcctgcctttttagattccttcaggtagaatgtactaacccctccttttctttgcggcataccctgaacattcgattgcacgtaggtgggaaagaagtttcttccacagaaataagcttaagggtgtttgaaacatttgaaaaattgagattgtgccaaaattccttaagtgtgtactgtgtgtctgaaacagtrtgagatgttttcatctcctggtcgggactatttgttcagcaacattttgcatttctcaagcatctcaaatattatcccacattttttaaattatgaagaactttcctgctcttcataaactcatatgtaattcaagggaagtatgtatttcatgtgtattaaacccacatgtgtttcaagggaaatttaatccctgtttgattaattccctctgtccgtcacggttttacttttaagagctatttgatgttcaagaagccattagaacccccataacgtccatttccgagaaaagtgat

SEQ ID NO: 137 - DNA, 599 bp, Homo sapiens
agcattctggaggttcaaaactcaatgtgtaagttttattcacctgctaagaaattattttttcaaagctagcctcaatatttattttaaatgagtgaacttcaaggcctgaaagaataaactaatactttacgaaatatttttgaagtataaagaatatattcaacatctttccatgtctccagattttaatatatgccttattttactttaaaaattttcaaatgtttcttttatacacaatatgtttcttagtctgaataaccttttcctctgcagtatttttgagcagtggctccraaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcaggtaaagtttactttctttcagaggaattgctgaatctagtgttaccaatttattttgagataacacaaaactttatgcttcgacaatgttattcctgaacactttaaatcctgaaagtgcattataatccttaattta

SEQ ID NO: 138 - DNA, 599 bp, Homo sapiens
ggcctctgtgccttccaccaggggctgccgagttgctgcagccaagccctgcggtcctgccagccagtgggaagacgtggcccctgggcttttaagaatgccgtgtagatatttctagtggctacatccatcctgaggctcatctgagtccccgtgtctctgtgtccctgtgacactcacttcgcatgtgttgctaagctctgctcccttgttcttgtgggcaggccagggtctccctcctttagaggcccctgtgtgcatctttcctcaattccacagggatattctggaggagcccgrggaggatgagctcacagagaggctgagccagcacccgcacctgtggctgtgcaggtgacgtccgggctgccgtcccagcaggggcggcaggagagagaggctggcctacacaggtgcccgtcatggaagaggcggccatgctgtggccagccaggcaggaagagacctttcctctgacggaccactaagctggggacaggaaccaagtcctttgcgtgtggcccaacaaccatctacagaacagctgctggtgcttcagggaggcgccgtgccctccgctttcttttatagctgcttca

SEQ ID NO: 139 - DNA, 599 bp, Homo sapiens
gcaaccttcgcctccagggttcaagcaattcttatgcctcaacctcccaagtagctgggattacaggcatgcgccaccacacctagctaatttttgtatttttaatagagacatggcttcaccattttggccaggctggtctcgaactcctgacctcaagtgatctacccaccttggcctcctaaagtgctgggattacaggcgtgggccactgcgcctggcctatccattttcttttatagacacagatttcatttcatatcctactttttctctgaaaatgtattttgaatgctgctsatggcaagagtagttcatgcagtggatgagtttcatacgatgcatttctgcagtcccctacagctgggcgtttaggtgattgacagctttttaaatattatgtcagcactgaagacattcatcttcccactctgcaggttagaagactgaggcttagtgatgtcggagggccccacagctagtgagtgaggaagccaaaatgtgaacttgatggctttttttgacttaccccatgtttgtgtgtgacagccatggctgtggcgggggtgagagtgggtacagagatgccagcttgggtt

SEQ ID NO: 140 - DNA, 599 bp, Homo sapiens
tgttctcacagatggggaaatgccacatagagactatgatttaccccagcctagtaacagacctatccaaagtgtgtcttttgtaccactcccccatagacaatcagacttgatagccaggttgctgtttgagaccaaattctgagggcccctagggattcacatgcatcagcatgagatctgggaccttctgctctaagggtgggcagctctgaacaggtaagtctttaacattctcaaagaaggcttcaaatatctggatttctcctctcttttagcctgtctagggccatttgtttrgaggaaaaatttaaaaagcaaatcacagtcctccagggcattcatgaaaccatgtatatgccttccttggcctcaggcttaaacatcccttccctggtctccaagattgcagcgtgcagccccgcccactctgcctagaggtctctctgtccctccagcctgaacaactgggacaaggggtgggacagagggaatgaaaagctctgcgggtgtggaaggtaaagaagaggtgatggggccatgctcagtagctcatgcttgtaatcccagtactttgggaggccgaagcgggtgggtca

SEQ ID NO: 141 - DNA, 599 bp, Homo sapiens
atatacactgagtgagtggtacccagtttggggttaaaataatgggccaaagagaatccaaagggcacataatctgccattagaaaatcaagctctgacaacatgctatattcttaagtaatcaaagacacagaaaggagttcagtctttaaatgaataccactttagggaaacaaccttcagctgagatttagaggctctactattccctacaaaatacttactagttacaaaaggaaaaagagtaactttacagtcaagaaggctggcagacagtatcttccatgttttcaaagttarcatcatcagtaatgagacaaatcaaaattgtatgccacctcacaggatatgattagaatacaacatcatctctctggtattcctgccaaagaatgcataacctgaatcttatcatgaaaatacatcaaattatggacattctacaaaataactggcctgtaatctttaagtgtcaaggtcatggaagtcaaagaaataatgacgaaccatcctagactaaaggagactagagagacatgacagttaaatgcaacatatgatactaaaccaaatccttttcctataaaggacagttcgaa

SEQ ID NO: 142 - DNA, 599 bp, Homo sapiens
ttcacaatagggtttgtgttcctgagaaactaattccgtggccaatctgacgggaggcagagctcaggcagtaatgccagctttgctgctcacctcctgctgtgcaacccggttcctcacaggccacagacaggtaccattctaccacctagggttaggggaaccctgctatacacgtgtaaagagcagatgtggcccattacctttccaatgagatgacctgccacatctgctactagcacttaggagcaatgccaacccagtttcaagagtcagagaagaacagccttaaagtgtgarctttaaaatcgcccctttctactccataattgctgtgtgacctttggcaagacaaatttctgtctctgagccgccaattccacatgtgtaaaataaggataaaagcagcacctaccaccccataagtgttattaagaataagatattaaaaacccaacttgagatacagtaagcgttcactaactgcacatttacagcccacaccccctttcacagagagtattagagcatcctggttaagagtatgaactccactaaaaatacaaaacttagccgggtgtggtggtatgcgcctgtaa

SEQ ID NO: 143 - DNA, 599 bp, Homo sapiens
agaaaatgagggtatgggcaccagtcttacccccaatctccagcatgggatagggcaacagatgaatccaaaaattatacactcagtgtgtcaaagcaggaaggaacttattcatacagaattccattctgcagatggacacactgagactcagagaggagcagggactttgcaaggtcacacaaagttactccccagctccataattctaggtggaggggagggtacttttaggagacttcaggtatttccattggatccaaatctgggctcctgttgccatggtaacccctcctataragtctagcttagtctgaggcagccatgagtgatcatagcttcaaactgaactgaagcccctagtttcaggtgggggagggcagatataaatagaaaccccttcctctcctagacttcaagctctgtgcagataagcaaacggtcgggcctgtgtgggcgtcaagagaggtgttccagtcccagctccgccaccagacttgtgtgaccttggaacaatgacctcccactctgggccactaattcttcatcttcagtattctggggatgagccaggagtgtgatatctgagggcctgactt

SEQ ID NO: 144 - DNA, 599 bp, Homo sapiens
taggcacctggggggcttaaagctacagatctttaccagctagcagagaaatattccaaacactgcatttgtaccaccagttcctaccagcagaacctcagccctgccacgtgtctgaaagggcatagctgggaccccagaatctgcaggcaggggctgtctgggtgggcaggagcattccactaggaaacgaagcttctggaactgcaaggcagggaccagccaaaatgcagacatctctagggcagatgtcactataaacagggcccacagaggagctaccattgccctccatgccaygcccatgaagaagccacccagagggtagacgctgcccttgagcaaggcgaggagacccctgaagtggctaagcctacgctggagtgccttagaatgtcctggcattgccttgagtatccagttttttgctacttggtggcacaggatttcaaaacagtgaccaaatcggataacggaaagagaatggttttgtgtttgtttgcatacacaaactgctttgtattgattgggctgtgaaatttgtgcttgtggcatttgctcttccaagctgatgtcaaatgcatccactattcatccaa

SEQ ID NO: 145 - DNA, 599 bp, Homo sapiens
ccatggctgggcctaaccggaacaaggacagggaaggtggaccacagacagctggagagctcagctgggcaaggctgccagagttccggagcctgccaggccatggaaggagggtgttctgtcagaagccaggccaaggctgtgtcatgggaaggaagcatcagcttttgagattttacagaatgggaaccacgtgcattggtgcacagaccatggagaatggcaggaaggttaggacagagaacttgagctcttgaaacctaaatcaaactctttcaggttccccagaccatctaggcrtatctgtacctgtctgtgtctgttttcccttatctgaagctaggataatagaaccatttccggaggctactaaaagcaatctctccggagcttggcatggtgcagggggataagcatagattctggagccagacaaacctgagtctaaatcccagctccactgcttactagctgtgtgactgggaaaatcatcccacctctctgagcatcgatttcctcaactataaaatgggagtgataagaatctaacgcctacctccaggagttgctaggttaggtatgattatgtttcagagggt

SEQ ID NO: 146 - DNA, 599 bp, Homo sapiens
gtggtggttgggcgcctgtagtcccagctactcgggaggctgaggcaggagaatggcgtgaacctgggaggtggagcttgcagtgagccgagatcacgccactgcactcgagcctgggtgacacagcaagactccgtctcaaaaaaaaaaaaaagaaatattaaagaaagttactttggaatagaagaaaaattgaaaataaataagctaaacacccattttaagatattagaaaaaacagaataaaccccaaaagaaggaagaaaataataaagaacagaatttgttaaaacagaaaayaaaggtacaatataaggaatcaacaaaaccaaatctagttcttagaaatgactcatgttattgatggctttctaataagaataattaagaaaaaaaaaaaaaagagagacgaacaaataaacaacattagaaatgtaaaagagagcataactatagatgtatcagggaggagaaaatattttaaaacttatgccaatgtattttaaaaaaggtgaaatggaaacattctatgaaaaatataaataatttaaatgattaaaaatgcctgtgtaactaaaaagaagtggagatcaaaggac

SEQ ID NO: 147 - DNA, 599 bp, Homo sapiens
ccaaatgctcagacttctgtggggaactacatctatgtacacaataccaggctggatattttacatctattttatggtattccacaacagcccaacagcacaggtcctgacatatatattttcctaagtcctaacaagaacttaggaaatgtttgctacactgctcagggctcttctaccttcaagcatgagctcaaaagatggtaggtattattatttcattttatatgtgaagaaagtattgcttggggaagtacagtaactagcttgaggtaacctgtaaatggtagtaccagaatraaaacctagttccaattctatgatcagttcattagctacagttcagaatagcattttttttgttttgttttgagatagagtctcactctgttgcccaggctggagtgcagggtgcgatctcggctcactgcaacctccacctcccaggttcaactggttctcctgcctcagcttctcaagtagctgggattacaggcgcctgccaccacacctagctaatttttgtatttttagtagagatggggtttcaccatcttggccaggctggtctcgaacccctgacctcaggtgatccacct

SEQ ID NO: 148 - DNA, 599 bp, Homo sapiens
cggcggggtccgggctgggagacgccacggccgccattagtcaccgaggtggggtgggaaagagaggttcgctgcggcttcaaggtctgggcacagccagtgggcgggcacagccagtgggcagccacagcagaggcctccgttatctgcagggcagagggctcggcctgtcccggggccccccagttcacccgccggcccggggccggagggcctggagggccctggaggagcgggctgcgttctgcgtctctccgcgatctctgccggaccggaactgacttggcagttctctgcgcwggcaagagaggagcggtaccggaagtgcccttgcgctgggggcccgcgagccgtggttccgggtctgcaatgttccggcgtgggggtggggaatggggaatggggtgtggtcacgtggggttagggccccctccaccccgcttgctcttcgcaacggaaattgctactggcctcagaattccctgagcaaacgttgcttctctatctccttactcctccggtgctggataagaagactccaatatttcatttattgactcatttaccacctgtttaccgagcacctatgtgcctctaag

SEQ ID NO: 149 - DNA, 599 bp, Homo sapiens
gggtttcactgtgttatccaggatgggctcgatctcctgacctcgtaatccgcccgcctaggcctccccaagtgctgggattacaggcgtgagccaccacacccagcccgtcaacatttttgttcacattattggaggatgaggctccctggaccattctcttgccttgcccacctgtacttggggagaggggcagctgtgtgcagtgcccgctttgccccatcctcacccatgctgctgtcctctctgttgtctctacctggtctttgctcactggatgttacaaccctggggcagggkgacccagtggactgctgtgtgcacagactcacccttttccacctatgaaggccgtagtcgtggcttggacatggatcccatcgcctgtcgtggttgacttcatgctctccacagttactttttcaacctgtaatctgcaaagcgaaagcatcaagagtcagtcctgtttcaacgaatcagcctggacagcaggagaaatgcctttgaagcagataattaacagcctaccaagccaccatccagtcccttgacccctcagctgtctaatgtgcccaggcctataagctcaaaaatcttga

SEQ ID NO: 150 - DNA, 599 bp, Homo sapiens
agtgtatatttagaaaaccccaccgtctcagcccaaaaactccttaagctcataaacaacttcaacaaagtctcaggatacaaaatcaatgtgcaaaaatcacaagcattcctatataccaattatagacaagcaaagcgccaaatcatgagtgaacgcccattcacaattgctacaatgagaataaaatatctaagaatacaacttacaagggatgtgaaggacctcttcaaggaggactgcaaaccactgctcaaggaaataagagaggacacaaacaaatggaaaaaaattccttgytcatggataggaagaatcaatatcatgaaaatggtcatactgcccaaagtaatttatagattcaatgctattcccatcaagctaccattgactttcttcacagaactagaaaaaactactttaaatttcatatggaaccccaaaagagcctgtgtagccaagacaatcctaagcaaaaagaacaaagttggaggcaacaagctacctgacttcaaacgatactgcaaggctacagcacccaaaacagcatgctactgctaccaaaacagagatatagaccaatggaacagaacagaggc

SEQ ID NO: 151 - DNA, 599 bp, Homo sapiens
tgcatctggttttgaggaggaccagctccttgaccaccttaaaaaaaaaaaaagtcacagtttcagaaataaatatttttatcttattggtattaagatactcaagttacattactcagaaagacatccatcacccttatatccactgaccaacataaatatgctgttgacatacttcctgtgagttttctaaactacaaagatgtggctgtgggataactgcttacattcccaatgaccatataatagagaaggagaaacagtttctacattcccatatggtatcttaaagatttaacrtttcaaagatcaagaaagtttactttctttcagattaaataatgagaaaggcagcatctcactgttttaaaatgacttgtagtggccaggctcgggtggttcatgcctgtaatcccagcactttgggaggctgaggcaggcggattgcttgagcctgggaggtggaggttgaagtgagccaagatcatgccattgcactccagcctggtgacagagtgaaaccccgtctcaaaaaattaaattaaattaaattcaaaaagcactatgggctactgaaataactctgctataagaatgtg

SEQ ID NO: 152 - DNA, 599 bp, Homo sapiens
ccaagaagcgtgggtgagtccacctagagtttgctagacccactgccacagaacctctggaagagcttattaaaaatataaattcctgaccctgtattccacattgaatcaacatttctgggagtgggatcagagaacctggctttttaaaacagagccatgtacttgggacggctgcaacataattgcagagtttaaagcctgttaacagggaatctgcatttttaacaagctccctctgaggattttgattgagaatcctgctctgtggaacagagggacaggcttcccagctcaggsgaaggctgagctccttgctggtactcctcagcctgtgaccacagcctcctctccaccgctcccagccacacaatttatgctcaactccccaacagccagaccggcccccttctcctctgcctgcctttgttcctgctgtcccgagagtccgcagagcctctcctccccttgccttgccctctccccctcactctatctactccctatgggtgctggaaccacaggcaccattacagaaaggggtgcctcagaaatcaagccttatgtgaattttgttcttggctcctccaatgaccttc

SEQ ID NO: 153 - DNA, 599 bp, Homo sapiens
gagaacagtcactgctaatttatatctgaattcctcagcttatgaaacccatgcccaattatttcatctacggccctcaagagaatcctgttaaagcatttcatctcagcccctaaagttttcaagatatgctccattagtttaagatatatactccctggtcttggtgatactaagtccctaaaatatagttcttaattttagaggaagagtcaaagaccacacaccaataacagctatgaactcttggaaacaatgatatacacacacacacatataattctccataccatttccacrgttttataggcccattaaggccaagttaagaaccctcagtctagatattttttaaaatctctctggggtctaaaattctagaattcaccagcttcatctgtcactgatgaccagtgacacagaaagcacactgctatttctggtttcattagtaacaaaagctaaaatgaaccacaaacagacaatgccaatgagatgcagccttatttcaccccagcctaaatcttctctctcagtggctcccagccaatcaaggagtgttgtagccatttaactacatgggtagtacacatggttaa

SEQ ID NO: 154 - DNA, 599 bp, Homo sapiens
actcagttttcctcaattctttccaatatttttttaaattatggaaaaactgaagaagtttcaaagaaaaaccaagaggtaaagaataggatggaaaagaggtaattttgaagaatggtttgaaatcaaatgaagtgtgacatgactattaacactgaaatatccatggggaggcttttgaatggagaccctgaaaagctgatgtatctctcaatgcccacagatgactcaccaagaagagagggctaacaaaaccctcaaagtttgttgttttacagaagagagatttctttattgtgmtggcaaaagttactggcataggaaaatgagggaggctgggtgtgttgtcattgaccatctgtttttctacagggtgtttacttatccatgatcacctgatcatattagaatgtctctaaaacttgactcagttactcaaagcttgaatcaattatctaccatttcaaaattccgcagtgccatcctgctcccacacactggtcactcagcaacattcaaatgtcttagacctggcatccagggccccaagaaagtacgtcggcttcacttctccacaactcctcaacaaaccagcaaat

SEQ ID NO: 155 - DNA, 599 bp, Homo sapiens
aaaatggtgacattattgcctagccaggagcaatgtgtggctttgcatgcagacaataaaagctgccggagagctggctgcacggccagtgcggaaaggtgccctgggatccctgctcctcttcaccccatggctgggcctaaccggaacaaggacagggaaggtggaccacagacagctggagagctcagctgggcaaggctgccagagttccggagcctgccaggccatggaaggagggtgttctgtcagaagccaggccaaggctgtgtcatgggaaggaagcatcagcttttgagrttttacagaatgggaaccacgtgcattggtgcacagaccatggagaatggcaggaaggttaggacagagaacttgagctcttgaaacctaaatcaaactctttcaggttccccagaccatctaggcgtatctgtacctgtctgtgtctgttttcccttatctgaagctaggataatagaaccatttccggaggctactaaaagcaatctctccggagcttggcatggtgcagggggataagcatagattctggagccagacaaacctgagtctaaatcccagctccactgcttactagc

SEQ ID NO: 156 - DNA, 599 bp, Homo sapiens
tctcaaaatatataaaacaaaagtggattaagctacaaaggaaaaatagacaaatccatcatcaatgcataattgatagatcaagcagacaaattagtaatcaaatagaaaaaatgaagcacatttcacaagtttaatctcataagatcttacaaatcatacttatatagagcagggcacctaataactaagtaattcacattcatttcaagcattttgggggtatttacaaaaacatcacatacttaaatcagtccataaacccacattgacttcaaggaatctttatcatacagactycaatctctgacgataatggaattaatagaaattattaatgaaaagataacgtaaaataaattagaaaaaaacttgctgacctcacatatgtatcacttatttgttgtgagaacacttaacatctattttcttagtaattttcaagtatacaatatattattaactatagtcatgttgcataataaatctcttgaattgtattataaaaaattaaaacacttttccaaataacctaggagtcaaagagaaatgatgtcagaaaatacctcatattactatttgtgtaatgtagataaaac

SEQ ID NO: 157 - DNA, 599 bp, Homo sapiens
atacccatcactacccttttctctgcattggcagttatagttccgtttgtattcttttggtgatcctcttaaatatctgaaatacacacctgactttacaaagccagacaatgctcagtctctttaaccccctctcaaattgccctctcagtcttccatgttcttgtgtctcatgcatcttgtttctgtggcccacaaattaatcattattattcatattgcatattaaatgcttgtttagattcacatgtttaccaatttgctcaccatttttccatctaatttcttccttttgggttscatgtccttgttactaaactacattctttaaaaattatttcggcggccaggcgtggtggctcacacctgtaatcccagcactttgggaggccgaggcgagtggatcacgaggtcaggagatcgagaccatcctggccaacatggtgaaactccatctctactaaaaatacaataattagctgggggtggtggcaggcgcctgtaatcccagctactgaggaggctgactgaggcaggagaatcgcttgaacccgggaggcagaggttgcagtgagctgagatcgcaccactgcacccca

SEQ ID NO: 158 - DNA, 599 bp, Homo sapiens
gctgtaccatgcaggctggcatgggttctgccagcaggctgttccctaacaccacaattgtgcgtctgtatgactcatccaagctccagagagtccagcgtctgggacctacactcagggaggggccacgttccttttctcctctccactgcacaggaaaacagaaaggtgaatgtgggagcatgcatgttagcgctgacagctaatgtttaaatggaattcctagccacattaaatatattcaacgtaggcttaaacacgtctcaacacatacatcccactcataggtcctaaggaccrgggctgatgctgctcccagctaataatgattcacagttgtcaagccccaagcaggttctccaagcacgtgtactccctgctgcaggagcagtgaagcaaaggtggacagctactgttctctgcaggatcttcagacaacctgaacctgcagactatgtgccctgccagccagcaaaagcaactattagatcattcaggtttctggaaaccccatccttcttctttgtgcctcctcaccccagagggcataaaatagtttattcgtcttgaaaaatatgtttattctgatgactctggct

SEQ ID NO: 159 - DNA, 599 bp, Homo sapiens
gtagagggaaggagtgcagtgtagaaggaggacgtacagggcttctaaaggactagaaatgttacattacttaagcagggtagtggaacacagtgttgctttattatgttttaaactatgcaaatattataaagcacaagggcattcaagacagatgggacatcatgggtatgggacctgaggtgggatggaacttgccacactggaggaacagagaaggtctgtgtggtttgagatttaggagtgaggggtaagtggtataagataagcatagagaggtagagaacagccaagtcaagsaggagctaagccattttggtaatatacatctcataggctctttaacagttgagaaaaatgtttcaggtggtgagtgttagcacctcttttacaatcttgtgacaaagaaagcaagttccagaccaaattctagcacacctaaagacatctattaaacagatattccttagtgagccaggcccaaagcagttcacacaaactattctagaagctccatgctgtcccagctctttctgcctctagttattccaaccaccacttatgaaaaaggttcactcataatagaactgacaatatcc

SEQ ID NO: 160 - DNA, 599 bp, Homo sapiens
cagtgacacagaaagcacactgctatttctggtttcattagtaacaaaagctaaaatgaaccacaaacagacaatgccaatgagatgcagccttatttcaccccagcctaaatcttctctctcagtggctcccagccaatcaaggagtgttgtagccatttaactacatgggtagtacacatggttaatctgttaactttctcagaccccaggaaaagaagaatggtcccactatgcaattctccttggctattacatatggattaagactgaagtttatatcaggcatttaatacatgycaaattttccaattaagattactttctgcagggagacagaaatgacattgagaatccaattttacctcagataggcataacagtgacttaaaagctgtatttatcttcctatgcaaaaaaaaaaaaaaaaaaaaaagactggtgaaagactagaagatttttaagggtcctcattaacgacaggtaatataactgttattcagcactactccagaagtctgaacttgcacaggtatgtggatattaatagttattaataagctacatagatgaagctggcccttctttatctgttagta

SEQ ID NO: 161 - DNA, 599 bp, Homo sapiens
tcatgaaatactgctccagaatacggagatactcccaagtccaacagtgaaaggccttcaaccactgaaaaattagatatacaagttcactcagactaagcaggcagctacagaccaaattgttagatacccccacaaccaaaaagggccttagcgtttatctaaccccctttacctcatccccagcaagtggctttaagtcttgaacatttctgatgacgggaaactctctttcatggacagcatcctttttcattgtgggcagcactcattataagacagttttatgttaaactatcygcttccttttagttccttgtaactcttaccaactctcaaaaatgtctcatttatgcctaatcttccccattccaaagcacaataagataacaaattagaagctggtgagaaatgttgccatagtgtgaggaaatgggcagtcactctcatatgctgctcatttgagtgacatttgtattaacttttcgacagggcaatatgtgctttctaagtgtgtgccttctgacccagatatgatacttcttagggcagtttatcctatggaaataacctgacaagtacccaaagaggtatgttcc

SEQ ID NO: 162 - DNA, 599 bp, Homo sapiens
atagagtctcactctgtcacccaggcttgagtgcagtggcgcaatctcggctcactgcaacctccaccttccgcgttcaagcaattctcttgcctcagcctcctgagtagctaggactgcaggcacatgtcaccatgcctagctaatttttgtacttttagtagagacgcggtttcactatgttggccaggcttgtctcgaactcctaacctcaggtgatccacccaccttggcctcccaaagtgctgggattacagacatgagccattacacctggctcctccttcatatttaaacagrtggttttgtttcctggatgtgactacactgatcttcaaataccctaccatgacccacaaggcccaggtgatataatttggctgtgtccccattcaaatctcatcttgaattgttgttcccataatcccctcatgtcatgagagggacccggtgggaggtaattgaatcatggggatggttacccccaatgctgctgttctcctgatagtgagtgagttctcacaggatctgatggttttataagggtcttttccctctttgcttggcacctctccttcctgccatcatgtgaaggtgga

SEQ ID NO: 163 - DNA, 599 bp, Homo sapiens
aaatacaataattagctgggggtggtggcaggcgcctgtaatcccagctactgaggaggctgactgaggcaggagaatcgcttgaacccgggaggcagaggttgcagtgagctgagatcgcaccactgcaccccagcctggcgacagaacgagacttcgtccccctcccccctcccccaaaaaaatgttgatcttcagtctgcgcattctgctctggattttaagcatcatttctggcagctctttttcaggaactgtctctgcaggtctttcagtgttcgcctctggagctctgctcgrgcccgcgagttctcctggcgttgccatctgtgcccttttccttattccccatcacctgtctctctggtgagctgcttccgatcagcctccaggtccacgatttctcttctgttgtccttttctgccttctcacctcatcctctgagacctgaatttcgatggctctacttttcaattatattattctttttcaaacctgggcgttcatttctagcatcctgttctatcctgtgtctgactgtgcctcccgcaccttctcccttctctcgccctaagccccgtgaaggcgccgggccttt

SEQ ID NO: 164 - DNA, 599 bp, Homo sapiens
ctctaggtaccctggatacaggtaattaacaaaaatgagcagagacatataatgataacccataacaaatattagctaagccaaacaaatctgaaatgaggcagggattcatcctaggaataaagagactcacccctgttttactgcgcaagacagtttaaaatcaggagatacccagtactgtaacaaatgagttttaaaacacaggatctattttgggcattctaaagacctgcctagaggaccacgctcaaagatcctgctcctccccattcttactccacaccccacagcaggagycagaaaaccgagtgcctgagaccgaagtgagacttctaggggctttaggaacaatcggtgggttctggctaagatccccagggtcccagtcttaattgagaaattagggtttaactggaaaactttttgtccaattaaatcagacaaatatttcgctgcaattctggctctcctaaagtttacatttttgatttaaaaaaatatgaagttgaaactgatctcaatacacaattcaaaaaggacaacaaatagggcacgcagagggagagatgagtgatggctcttggtcagatttccct

SEQ ID NO: 165 - DNA, 599 bp, Homo sapiens
agaccagcctgggcaacatagtgagagcccatctctaaaaaaattttaaaatttaaaaataaaaaatgtttaaaaataaaaataaaatggggctaaaatgcacacttcatttggtaaacaagatgacatgtgaaaaaatgacacagatatatcactggctacattagttttccattgctactatagcaaattcccacaaacttagtgatttaaataatattctttattattaaacagttctggaggtcagaagttccaaaatgggtctcacaggctaaaatctctagaggagaacccctygcttgccttttccagcacccagaggccccctgcattccttggttcatgacccccttccagctgcaaatccagcaatggtggattgaggccttctcacattgcatcactcagaccttctcttctgcctccctcttccatcttttttttaaaatagactatttattttgagatggaatctcactacattgcccaagctggagtggagtggctggatcatatctcactgaagcctcgaattcctgggctccagggatcctcctgtctcagcctcctgagtagctggaactacaggcatgcac

SEQ ID NO: 166 - DNA, 599 bp, Homo sapiens
ctgaggcaggagaatcgcttgaacctgagaggcggaggttgcagtgagccgagattgcaccactgtactccagcctaggcgacagagtgagactccgtctcaaaaaaaaaaaaaaaaaaaaaattccctaacatcatctaatatctaatctgtcttcaaaattttctagttgtcccccaaaacatcctttatagctgtttttttcaacccaaaatccaatcaagactctcccattgcattcagttattgtgtctctttagtccagaacggtttctccactttttctttttcataacattracattttgaacagactcagaccagctttcttaaaataccatagtacattctggtttgtcttactgcttccttgtggtgtcattgaactcgttcctctgctcttttattccctaaaaccaaaactcagatcttaaggcttcattaagggtaactttttgggtaagaatagttcagagttgataacgtgtattttgtactgcctcatgccaagcactatatatatcatgtcaagttgttccactataagtgataagagttggattacttagctacagaatatgcctttgtaaagatttttg

SEQ ID NO: 167 - DNA, 599 bp, Homo sapiens
acctcttctgcccaagcactcgcctgctggcaactctggctggagggcttggcccagcttctggccccacagccccctgaggtccatgcagccctgtgccagccaggcctacttgagcacgagcatggcctctgtgccatccttggcggtcaagtagaggccatgtgtgaggtagtactcccgccgcaggaaggcatagaagtaatcagagatgagcaggatctggggggagagagagggtgtgaggcgctgctgggtacctccccctaaacccaggatccctgaactgctggcaggtgktgttggaagacaggaacgcccctgcctggctctggatcacaatcactggagaggccctcctcagggcccccacctcagggcccataattgaccctgcaaggtctggcctctcagaagctgcctggactccccctgcccccacccatggtctacacctccatcccaggctctggcctcgaatgccagcctggtgcctgtgcaaccagaggcctctgccccatcctcccacagccacttccatcccagccaggtccacatcaaactcctcaccttccctctgtcaaatacacttctcccac

SEQ ID NO: 168 - DNA, 599 bp, Homo sapiens
gcttttcaggtagctggtaacaacggcaacatttctatgaaaaggctggtgaaaatgcggtgacaaaaaaacagactacagcctcctgcagcactgcacagggactggggtggcctgaagggccctgttcctacaaagctggcactggaaacaaatcttgcagggggcaggagagcttaggtagagcttcatacccaggcagaagagaagacagcagggagagcaggtctcttcatattttctgtccccacaactgtgtgggcctggcttaggtcataaaatggtgacattattgcctasccaggagcaatgtgtggctttgcatgcagacaataaaagctgccggagagctggctgcacggccagtgcggaaaggtgccctgggatccctgctcctcttcaccccatggctgggcctaaccggaacaaggacagggaaggtggaccacagacagctggagagctcagctgggcaaggctgccagagttccggagcctgccaggccatggaaggagggtgttctgtcagaagccaggccaaggctgtgtcatgggaaggaagcatcagcttttgagattttacagaatgggaaccacgt

SEQ ID NO: 169 - DNA, 599 bp, Homo sapiens
tgaaggtctcctcatctaagagtgagtttagcaaagggcaccaaatcaaactggaagtgaactttttttttttaaacttttactgagatataacaaagatactattaagtgtataaatcttaagtgtactgcctgataaatttttatgtattatacatacacccacacaagcaccacccagatcgagatacagattatttagggcacctctgaaggctccctcatacctggagggaaccacatttttttgttagaagacaaacaagtctgaatccttcattttactgatgaaagcaggastcctaaagacggaaagtaatttgcccaacaccagttttatatccgtaaaactgggtatagaactgtgctgtccaatacagtggctcccctgtggctattgagcattttaaatacagctagtcaaagctgagatgtggcacaagtttaagatttcaaagatttagttgaagaaaaagaatataaaatatctcaatttttaatactgctttcatgttgaaataatatttcagatatactgggttacatacaatctattaaaatttgtttctttttactttttcaaatgtagatactagaaa

SEQ ID NO: 170 - DNA, 599 bp, Homo sapiens
ggaatgctaccatgcttgtttgtttatgtattgtctatggctactttctgtgctatcataacaaagttgagtagctccaacaagagactatataacctgaaaagccaagaatatttactatctgccccttcagagaaaaaaaatactgctgattcctgctctaactcaagttccatctcctcttagatgtttctgagttcctgtgacattttacctacttttgatatattgacaaaagttactgccctgaaagaataattatttatgggcttgaaattaaggggagagaccaagacttaytacttatctgtatactcctgttgtctatcaagtctacgacacaatgctcaataaatgctggctaaacaaatcacagaaaacaaacctgcatgctggacattttgatttgcttgcagctgaggggctcctgaattcccaggggtggttgctgtggtcgaaggcacctgaccttgcataaagttcggattggcctgtcctgctgagaagccaggtgggaggcgtttaggcattccttaatagaaaacaatgagtaaggcagttacctagcaaatttatactcttgcttagatatggaatga

SEQ ID NO: 171 - DNA, 599 bp, Homo sapiens
atgaatttctgtgcaaattaaaatttagatttaaaaccatctcaaaaacccaagaggactttttcttagaacttaacataattctaaaatctttctggaagaataaacaagtgggagtattgaggaacattttgtaaaagaacaatgcagggggtccggggctaatggctctattactagattgtaaaggatacaataaaggaatgtggttcttacacaagatggaaccctacagaaacccacagggccaactacaaacaacgacattatctcctgggccctcccaagattcctataatrtacacagggccacatttgatctcatagtattaatgagatcactagggcttatgtggcttcttcctctctgattattcatgtatttcttaaagaggcaggggcttgagggctcagggaaaagcttatgccagggaactatctgtttggctgccagctaacatatttccgttgtcaaagataggtccagtcccctaagagattcctagggctaatgctcagcagtaccaaactgcattttttttcttttacattttattttatttatttttttgagacggagtctccccctgttgcccagg

SEQ ID NO: 172 - DNA, 599 bp, Homo sapiens
tccaaagtacttactgctacctgtatttgtttatttatttactgtgtctccctccactagattataagctttttgaagttaggaacgttgtcttgtttgctattgatctccagtgacagggtcagtacatagtaaatattgagcagatacttactacatagatgaacaaatccccatcccattggataagttcagaccctcatcatctcttgcctgggccatatcccagctgcttctcatatttccctgacttaaattctcctcccctcaatctacctcccctaaataagacaaatcagktcatgtgtctccccttcccaactggataatcttaaggctgacctctgtgagaagccctgtaactccacatcctaccgcttctcaataaagatctagtcttcttctttccctaataggaagacttttggtagctcttgagatgaagacccaatcttgaatagcattaaatcacttaatcgaaaatctatttcctttgtggttcactttcaatccttttgattatggcaaggaacaagtttcagtctgctctctaacgcttcctaatctccttctttaatggagaaagcagtctcagagcc

SEQ ID NO: 173 - DNA, 599 bp, Homo sapiens
ctttcaagggcccagcttatgccacagggccagatgatggtgaaccccccgagccaaaatcttgggccctcgccccaaaggatgaccccacccaagcagatgctttcccagcagggcccacaaatgatggcgccacataaccagatgatggggcctcaggggcaggttttgctccaacagaacccaatgatagagcagattatgaccaatcaaatgcaggggaataagcagcagtttaacactcagaaccagtccaatgtcatgccgggaccagcccagataatgaggggaccaactccraacatgcaaggaaatatggtgcagtttacgggacagatgtcaggacagatgctgccccagcaagggcctgtgaacaacagtccatctcaggttatgggcattcagggacaggtcctgcggccaccagggcccagcccacacatggcccagcagcatggtgatcctgctactacagcaaataacgatgtcagtttatctcagatgatgcctgatgttagcattcaacaaaccaacatggtcccccctcatgtgcaggccatgcagggaaacagtgcctcgggaaaccacttctcaggcca

SEQ ID NO: 174 - DNA, 599 bp, Homo sapiens
ttacttctgttgaaagaataaaaaggctgtcttcagtgctgttattttcaacaaaaccacatcttaaaaaaattattcctgtgctaacagaagaagtgaatcctaaatgctttacttcattcacaagtacattgaaatattgattatgactacatgagacctgaatcttgggtggccaggcacttgatgttgatggagtttttctcatttcatctgaatgtttggctggtatagacacagccattttcagtgcatagcacagaaaatcaggctcttagtacagtcatggctcattcagaycctacacaccgaggagctccaggttttttgatgctttgctttttgcttccttaagcactgtgccccttttgcttatgtcaagtaatgaggggctgccccctgccccaggagtgggcatgcctgagctgtttgtgcatagtctgtgccacctagatgagtattgcttggctatactctaaaggtagcaacattttcttgctgaaaaatctaacttccctaatttggggggcttgttttaggttatccttctcaaccagttgaacagaggccacttcagcagatgcctcctcaactcatgc

SEQ ID NO: 175 - DNA, 599 bp, Homo sapiens
gattacaggcgtgagccaccatgcctggctaggtcagctttctatgtctattttaaccagggaggttcactggtcacagggaacaaacagcctaccaaaaaggaagcaatcactgggagctaccagatgctatagcttgcctctagaagtcaggcgtaaaaagagaaacagaatacaggtcatgagggtttccattaaaggaagggaactattaatatacaaaatttgttaagacattctaagtagaaagaatattgttctggaaaaggacatcagttcgtatcattccttaaaaacagktccattgcttcccactagtttagaaataaaggccaaacttctttttttttttttgaatcggagtgtctcactgttgcctgggctgcagtgcaatggcgcgatctcggatcactgtaacgtctgcctccgggttcaagcgattctcctacctcagcctcccgagtagctgggattacaggtgcttgccaccacgcccggctaatttttgtatttttagtagaaacggggtttcaccatgttggccagactggtctcgaattcctgacctcagacaatccgcctgtcttggcctcccaaag

SEQ ID NO: 176 - DNA, 599 bp, Homo sapiens
tgaccatgggccttggtcagtgagggtctactatggcccagccagttatatcaagattgtggaaggctgggcgtggtggctcacgcctgtaatcccagcattttgggaggccgaggtgggcggattgcctgaggtgaggagtttgagactagtttggccaacatggcgaaaccccatctctactaaaaatacacgaaaaactagccgggcgtggtggcgtgtgccttaatcccagctactcaggaggctgaggcaggggaattgcttgaactacggaggtgaaggttccagtaagccgakatcccaccactgtactgtagcctggagacagcgagactccgtctcaaaaaaaaaaaaaaaaaaaaagactgtggatacatacagtcatgaatgcaattcagaataagcaacggcctgctctgagcccaagtgagcctgaaggcttcatggaggaggtgggaactgagtgatttacacacagaatgacagtgacatacagggaaatgactatgttttgattcctttagtttctcttgtactatcagtgttttaacttaatcttatgccagacttaaactgtgtttcttaaaaacgttttg

SEQ ID NO: 177 - DNA, 599 bp, Homo sapiens
ccaccacacccagctaatttttgtattttaagtagagagggggtttcaccatgttggtcaggctggtctcgaactcctgacctcaggtgatttacctgcctcggcctcccaaagtgctgggattacaggcgtgagccactgcacgtggctgaggctctgtcttttcactattataacctcagggcctgacatgtagtaaatatttaatacatatttgatagatggttattcatttgataattactgattggtacatctctgtatttggcaccatactaggcaccagagatacagtagttmgcaaaatcagacataatctcctgccctaaccaagtatacatactaattacctatgtcacaaactggcatagttatagctgtggtattatgaagaagaacctggtgctgtgattccccatagcagtgacaactgagctaagataagtaggaagggtgggacgttaaggagaagagaggtgggcagagagaagagcactccctgcagtagctgcaacatatgcacaggcttgtgatcagaggaatgaggacggcatgtccgacttgctaagggaagtgggatgaggctgggagatgacagg

SEQ ID NO: 178 - DNA, 599 bp, Homo sapiens
gccagtccatgctccaactatgaaggcagggggcaaactaccttgttcacactatccagacttctcctgaactctaaggaagattttctaaaccagccttataaactcaaagtggaactccatgaccattgtatatgcttgtatacaataaatatactgtgtatattcattcaactgacaattattcattaagtacctgatacatagaaggcactttggtgctgaggtaacatcaataaacaagacagaccaaatccctgcatccttggagcctacagtctaactggcaagagacacaaytaacaaacagacaagtattttttcaggaagcagtaaaagctaacaagagaaagtagggtgagagaagagaaagtaagagataagagtagagctactttagataggataaatggggaaaccaaaacatgtacacgtgggccgggcacagtggctcaggcctgtaatcccaccactttagaaggccaaggtgggtggatcacctgaggtcaggagttcgagaccagcctggccaacatggtgacaccccatctctactaaaaattaaaaaaatagcagggcatgtaatcccagctacttgg

SEQ ID NO: 179 - DNA, 599 bp, Homo sapiens
ctcaggagtttgagatcaccctgggcaacatagtgagacctcatctctacaaaaaataaacagaaaattagctgggtgcagtggcgtgcgcctgtagtcccagctacttgggaagctgaggtgggagaattgcttgagcctgggagatcaaggctgcagtgagctgagatcatgccactgcattccagcctgggtgatggggtgagacactgcctcaaaaaataaataaaaaataaaaaaagttattcaacaacctcaggcaaaacaattttctcttttgttattaactcagctaaactkaaacttctgtatccttcattcagttatacattctaatattaagataatggtcattcaacacctgtgaaaagaaggaaaaaagagataatggtcatagaggaatatgctttctcaaaatgagaaatatctaaggatagaggaaagtgcctacatgttgcctaaagccccttccagctcttaaaccattgtacattgaatgttaacttttccagacatggagttcacttgttctaatgtggaaaaaaaaacagggaaaacttgctaaaaggaatgagataacacaatcataggaatttaga

SEQ ID NO: 180 - DNA, 599 bp, Homo sapiens
gaggcattgtcaaaatgaaaaataaatactaaaaaacttacacttagaacatgccaaggaaaagatggcaatctgctccctaaatgtgatctgaggccacgccacagcaaggtttgagggctcacttttctctgtaattaacaatggaccccaaaactttagttattttaacacaggcaccaaaggaatatgctaaattctctcatttcaaacaaattgatgtgaaatagtataaaacaaagagatgggtcctgatattaaggatagattgattatttttcctttgagggaaactgatgrcaatctcctttgcagaaaatttccccccattattgcgcttgtctctttggaagagacaggagattagaaacagttcatgggagcttcaaagtgatgctgtattaaatcattatgctttcccctcataaggtttgctgtgattctgacaggtttggtaccaaaaaccatactctaatggaatctttctcaagtatcagttcctttttggaagggctaattttagaaaacttgtaataaggagaatctcagaacccacttatcatttacaacacaggccttgatgtggccagatcacctag

SEQ ID NO: 181 - DNA, 599 bp, Homo sapiens
atattacatgtattcaacataactaataagacatgacttgaatgcttttttattaagcagatcttatttaatttagtcagttttgatccaacccacatgccaaatgcagcttgccaaagtaaaataccctatggacattaggatcaaaatgcctcctttttaaaaagtaaagtctatttataatccaatcctttaagttgaaattaggccttttataaattgaatttcatgtattctacagaatcaacagacctgcttaagaactctctgaccactctcagaaaagttagtcagtgggayccctctccattcctcccaggacctaaacaaaaaagaaaaagggcaagaggaagaagagaaacatcagaagaaaagactacatataaacccttataaacctattcaaaataaatttttgtgattggccaaattccttctctctaccactgtttctgctaattttactctagaaagagttgtaaaaggccaggtgctgtggctcacgcctgtaatcccagcactttgagaagccaaggtgggtggatcacctgaggtaaggagttcgaaaccaacctgaccaacatggcaaaaccccatct

SEQ ID NO: 182 - DNA, 599 bp, Homo sapiens
gaatttattactcagggaatgcaaactcctatgcctttaggggccaggcaggtagtagagaatagaagtggactggtataagacagcaggagtcctgtcaacagtgccaaactgcagagcagatgccctatttaaaggcatttaaactcggacttttgtttaagacactgcatagcaactcaaaaccattataggccaaattcagcctgcaggcccccagtttgcagaaagaaggcaataaatgtatacaaagacccctgggattcttataagaagagttcagatgtgtctgaggcaagmaggcatgttcctgctctcaatgccttctggacatactccccaaagactgatgacctcccagtctcagctatgctgctgcatctcactgacgggaagtgtgatatgcccaaataatttgtaataaataacacaaagtattgatatcatgatggatttagctttttgtttggattcaattttaaaatacataaattaggggtagattaaaggttactcctaaaataatgtcgctctcattttcatgccaaaaagaagttgaaataactagtgaggaagaaacaaaacataccctctgtaca

SEQ ID NO: 183 - DNA, 599 bp, Homo sapiens
actttccttcccagtatcctggtttccccctcctggctctgagttatcttgatcaccaagagctactccagggctgcctgctgggtctaggaagtgaaaccccttctcttcctgatcagctgccctttggggagcaggaggaggcagcccagataggccttgcagggttccctttggtgaccctgactcccgtggccatttctctccttccttatgccctgggccacgcctgctgacccagtctggaataatctgctgcttactttgggtctctgctacttccctccaggtatagtcacrggggaggtcctaggctcattgtccctaataataatcccctgaccctttggttttagggttctagcaattccagtgtgggttgacacaggaaaaaagtgggcaagtgacagaggttcttgtagctttggtccctgggtcctccagaatgtggaggaaaggctgtcttaggacctgggaaaagatctggaagccattatagggtgagaggacgggggcacccacaaatgaggaaatggcagggtcaagttgggcgttatggagtgcccctcccttctccctgtctcagtgctgttaggggc

SEQ ID NO: 184 - DNA, 599 bp, Homo sapiens
agccagaggtagcaagtattagttagactccctgagtgcagaaccctctgacttagccactggctggatgcatgacttggagaaagccattcccattctccaggactgaatggcctcttttgaaaaatgagaaaatgaccttaacagggtggttttgaagaaccctagttctagaaacaaatgaggatgtagtaagctctgagcttctccccgacctgccttacctcttccctcctacaggctcctgaccatgaagcccttcacagctaatatgcagtcagatataaaagtccagattcrtttggagaagaatgtaggtggcagatatgagcttgcctttgggaactgcaggctcttgcccgaggctatttggatccaaactggagtccagtgagttctcaatctgtctttctctgtgtccagctggttatgatgacgtccccagtgagatttcacctgaaagatgtcaccagaaaaacaataccctaccaaattaggcttttgtgtgtatagtgatgtgtgtataataaataggactttctgtgtctttaaaattttgctttttacaaaagtaataattctatcgacatgaaaagaga

SEQ ID NO: 185 - DNA, 599 bp, Homo sapiens
gtactgttatctccccctttaaagatgaagaaagtaatgctcagagaggggaagttatttttccaaggtaatacaactaacagttggcaggattaaatttaaaacctggtttcttcccgggaggtggagcttgcagtgagccaagatctcaccactgcactccagcctgggtgacagagcaacactccgtctcaaaataaataaataaataaaacctggtttcttagagcccacattctgaattacagtgtacgaccatcaaaaaatacatgagaacatgcatatagctattgaatacayagaagatcctcagcaaagttttcttctccccgccccccccgccccacctgtccccctcatagttcctcctttcattcttcacccaccagggcctccaggtttatgatgaaactgaagtttccttggggaaattcaccttcgatgttgagaaatcggagattcagactttccacctgcaggtgtgtttgtctctagggtgaaggtgccaagggagtggggcagtgagggatgaatgatccaggaggagggcagcccttggcatgatccatttgtctccccagaatgaccccccagctgcc

SEQ ID NO: 186 - DNA, 599 bp, Homo sapiens
catgcctgtaatcccagcactttgggaggccgaggtgggtggattgcctgaggtggagagttcgagaccagcctggccagcatggtgaaaccccgactctactaaaaatacaaaaattagctgggcatggtggcaggcacctgtagtcccagctactcgggaagctgaggcaggagaattgcttgaacccaggaggcggaggttgcggtgagccaagatcatgccattgcactccagcctgggcagcagagcgagactcagtctaaataaataaataaataatgaaattatacatttccyactgagaactgctttagcagcatcctacacattttgatacgttgtgtttttatttttgttcaatttaatatatcttgtgatttatttgttgaccatgggttacttagaagtatgttgtttcagtttgaagtattttatgtttttgtagataccttattgctattgatttctaatatagttttactgtggtcagagaacatactctgtaggatttcaatctttttatatttgttgcgatttaatttatggtccagcatatgatctgtcctggtgaatgtttcatatgcacttgaaaagaa

SEQ ID NO: 187 - DNA, 599 bp, Homo sapiens
ggtgagcgtggggttttcagaacttatacctctttaagggtttttgttttttttttttagacagagattcgctctgtcacccaggcagatcttggctcactgcaacctctgcctcccaggttcaagcagttctcgtgcctcagcctcctgagtagctggtattgcagatgtgtgccaccatgcctggctaattcctttttgagctaaagaaggaagagtttaaccctttttgagattctttcaaaatagattgatcagctcttcaggtgtaggaagataagacaggagaagagaggcatrgaagcagctggtacagaaaactgtctttgctattagaattaggtacacttggctgggcgcggtggctcacgcctgtaatcccagcactttgggaggccgaggtgggtggatcacctgaggtctggagttcgagaccagcctggctaaagtggcgaaaccccgtctctactaaaaatacaaaaaattagccgggcatggtggtgcatgcctgtaatcccagcttctcgggaggctgagccaggagaattgcttgaacccgggaggtggaggttgcagtgagccgagattgcaccactgca

SEQ ID NO: 188 - DNA, 599 bp, Homo sapiens
agcaattctcctgcttcagcctcctgagtagctgggattataggcttgcaccaccatgcccggctaattttgtattttttagtagaaatggggtttctccatgttggtcaggctggtctccaactcccgacctcaggtgacccacccaccttgacctctcaaagtgttgggattacaggtgtgagccaccacgcctggcctagggtaaccattattaaaagcaaaatttgtggaccatttttctctgcacataccaaagtatagtagtaatttaggaacaacaagagcaaagggtgactsttgagattttgagcctggatgcccaggagagagctctgtgctttttcttgattcctcctaacaataatttgttaaacaggagggagaggacagagcaggtaaatgataatatcagtaactacttggaaatagtgattttcatactgcttttaaaagaggaataaactggtttcagtagatgaccttatcccaagagtaattgagttccccctcacttttgaaccagcaactttaagatttaatttacaatattttcttctgggcatggtggctcatgcctgtaatcccagcactctggg

SEQ ID NO: 189 - DNA, 599 bp, Homo sapiens
aacaatgtgtacccagtgggtgcaaagaggcagggggtgctcagaggagaagcagcagaaccagagtgcccataagccacacgtcctctgggaaggaagggaaagccctcttgggacagacacactcaattttgacttacaaagcagaaaccacaggcctaaccctgagtattgacatctgtgagcaagtggattgaaaagaaacagccaaaactgaaacatctgcaaacacgtcttaaatacagtacttcgctttgaaacctcttacacatttgtgacaaggtttgttcacatatctastttgggagcatgtgcccagaaaactttatacccccattgaaaatgaaacttcaggccaggcacgacggctcatgcctgtaatcccggcactttggaaggccgaggagggtggatcacctgaggtcaggaattcgagaccagcctgggaaatatagtgaaaccttgtctctacaaaaaatacaaaaattagccgggcttggtggcctatagtcccagctacttggggggatgaggtgggaggatcacgtgagcccaggaggcagaggttgcagtgagccaagatcatgccactgcacttc

SEQ ID NO: 190 - DNA, 599 bp, Homo sapiens
ggctggagtgcagtggtacaatcacagctcactgtagcctcaacttcccaggctcaagcgatcctcccacctcagcctcccgagtagctgggactataggcgcacatcaccatgcccagctaatttttgtattttttgtagagacggggtttcactgtgttgcccaggctggtctcaaactcctgggctgaagcgatccacctaccttgggctaccaaagtgttgggattacaggcgtgagccactgcacccagcttagccagattcttttcatgtatcatatggaatcctcatgattayataaagcaagtcctcccatcatcctcattttaaggaaaaggaggcaaagtgtgattgtattcattgtctgtggctgtgtcaggaattaccccaaaactacaaagcttaaaacagcaataaacatttattacctcaggtattttctataggcaaggaattcaggagctgattagttgagtgattctcaccagaaacatctcataagtttcaagatgttgaccagggctgcagtcatctggaagctggactggggttggaggatacagtttcaagcgggcccgtgcacacggctggcaaag

SEQ ID NO: 191 - DNA, 599 bp, Homo sapiens
gcccaggcaggtcttaaacacctaggctcaagcagtcctcctgcctcagccacctgagtagctgagattacgggtgcaagccaccctgcccagccatatgtgtactttttaaatttttcctcctgttgcccaagctgttgcacaaccatagctcactgaagccttgacctcctggactcaagcgattctcctacctcagcctcccaggtagctgggactggcacacgccactgtgcccagctaatttttttgatttttaggcgagatgaggtctcgctatgttttccagattggtcttcractactgagcttaggcaatcctcttgcctcagcctcccaaagttctgggattagaggtgtaacccgctgcacctggcccatatgtgtactttttaattgatctgagttctggacttttgataccatggaattctactgatgggaatttgggttgatggtacggggaacctctatatttatttactaggatgggaggaagtacaaaggttatgagtgaataaaattggtaagtattttagagcaaaactgcttctctgagtaaatttagtaaatctgcctaattatgaagttaaattgat

SEQ ID NO: 192 - DNA, 599 bp, Homo sapiens
tgaagtgggaggatcacttgagcctgggaggtcaaggctgcagtgagccatgatcgaaccatggcacgcctgggtgacagagtgagaccctatctcaaaaaaaaaaaagattacatctcctcaggtgttcctgaccacattagccagagtagcatcccttgccccacagcctagtccctgtatcctgcaagaatcttgctcacttgtttaagtctctttcaccagaatgtgagctccctgagggcagggacctcctcagttgtggtcaccagtatattcagctcttctgttgacctgcartgtgttcttgggcaaaacccttctcctttccaaacctgtttccccatcactaacatggagatcatactaacccatgaatgggagtttcagaagagagcatgtgtctgaaagcaatatatgacccatagtaggcgctgagtagggaaattatacctatgtgtcctcccattcctgatagatgtgtgagatgtgtgtttacttttcttttttctttctttcttttttctttttcttttttttttttttttttttttgatgagtcttacactctgttttccaggctggagtgcaatggcgcg

SEQ ID NO: 193 - DNA, 599 bp, Homo sapiens
tcaatagcattttaccatatatgcaagggattatttctgggctgtttattctgttggtctgtatgtctgatttgccaataccacgctatagctttgtagcaagttttcaaatcaggaaggtatgtgtcctccaactttgttctttttttcaagctgcttttgactattcagggtcctttgagattccatatgaattttaggatggatttttttatttctgaaaaaaagaagtcatttggattttgataggggattttattgaatgtacagaatcaataaaatccatattgatcaatacasattcaatatctgtatcaatctggggtagtgtggtcatcttaacaatattaagtcttccaatccatgaatgtggatatctttgtgttttctttgatttctttcagcagtgttttgtggttttcagtgtacgagtcttttgccttcttgattaattcctaagtattttttattctttgtgatgctattgtaaatggaattgttatcttaattttctttttatattgtttattcttagtatatagaaatggaactattttgtgttgactttgtatgctgctactttcctgaattcacttact

SEQ ID NO: 194 - DNA, 599 bp, Homo sapiens
acaacacacattcattttctcacagttctggaggctggaagtccaagatcaagatgcaaatagggttggtttctggtgaggcctgtcttgctggcttgcagacggacaccttctcgatgtctccatacagccttttctctgtgcatgcagagaaagagatctgtttcttcttataaggacaccaatcccatcagtttagggccataccctaatgacctcgtttaaccttaattagctctttaaaggccctatctccaaagacagtcgcactgggggaggagagggcacaatttgatctaygacactatttttctacttttttttttttgccagtttgatgtgtagttgagtgataactacttgttttagcttgactttatctggtgagtgggaagtttgagcatcttatacttttgttaaccattttgactttttttcttggagacagagtctcactgtgtcacccaggctgcagtgcagtggcatgatcttggctcactgcaacgtccacctcccaggttcaagcaattctcctgcctcagcctcctgagtagctgagattacaggtgcatgccaccatgcccggctaatttttgtat

SEQ ID NO: 195 - DNA, 599 bp, Homo sapiens
gtgacaagacctgatgcttccaaaggacggctctggctgctgtggggagaagggacagcagggggcgaacacagcctgcggaagcctcctacccctgccctgccaggtgctgctctcggtctgaaggaaacaagcttccctccttcactcactgcttgatgtaactacctcccaaccctttggactcagctccagagcccgcaggaggcttctctgacatcagcccctctactgggcaccgaggcccccagccttcgcagcactgaccacaatgcagtgcaactgtttaggtcacgttcrtctcctcctccagacgcaaggcaccctggaatcaggaaaccagaccagactttatagccccagtacctcgcataggataggctcaataaatactgaaggaacaaataaaaagcatttctgcaactatgtttcacgaaatcaacctttttaagtacaggagggggacatggcctcacagtccacagtgtgaaaaagagctaaggtttaggtgatctgaaactcaacgagaaccagatactaagatctggctgtttaaaagtaaacatgggctgggcacagtggctcatgcctgtaatccc

SEQ ID NO: 196 - DNA, 599 bp, Homo sapiens
agtgaaatacatggcttaggagaggttaaggaacgtatcaactgtaactggtaaccagtgggctagaatttgaactctgatcttaccctgaagtttgtgttctttctgcctctcctcctgggtgcttaccactcagtggtaggcactcgaagcattttagctgcataaataagataggcacttaattagctggcctaggatatcgtaaaggtctttcccagctctagcagtgtgtgactgactccactctggggaaatagagcttcttcacttaagcctcacaggaggaggaacatgtcrgggaagggaagaagggaaggaggggcggttgtaaggacacctatgcttccatcattagcccatcttaacagtacataggaaatagcctgaactagatacacaaatggaatgtaccaatcaacaaagagattacatcaaaaagaaactgggaataacaatttttaccatttattgaataataattatatgctaggttctgtgctaagaatgttatatgtactaccttattgaattcttacaactattattgttccatattacaaaggaggaaaccaaagtctggagtggtcaagtaactt

SEQ ID NO: 197 - DNA, 599 bp, Homo sapiens
ttttcattgagaatcctgaaagccacctttccataccaaattcccatctgtctctaccaaattaccagaaaaagaaaaaatgagaaatggaggtctcagattccgtcatcacagcaggtagaaatcaaagtcactgaatccatcaaattagtaagcataccccactgtcagtgaacttcagctgatcagttatgtattgagaaggcaaataacacaattgcagaacatgttcaaaatactagtcctactgctatggtgaacaagctcaatccaatcaaaagcacctttattttggtctcycactgtctaaatttactctctagggcttgttctcccgctgtccctaggtgagatgtagagcaagccctgccctgggcagatgaagtccccgcccaacacaggaactactccttccagccgcgtttggagagaggccgccccttccactgtgggcccgcggggaacctcgctctctgcagggggagagcagcttatgtttggcctcggccttattttatttgcccacttggtgctgtcagctctctttcccaagttgctgagaggggcatgaggtggggagcagaaattcatcgttcgcc

SEQ ID NO: 198 - DNA, 599 bp, Homo sapiens
gaataaatgaaacaagattggctcagtagttggccgggcgcggtggctcatgcctgtaatcccagcactttgggaggcataggcaggcggatcacctgagctcaggagttcaagaccagcctgggcaacatgatgaaaccctgtctctactaaaaatacaaaaaattagctgggctcggtggctcatgcctgtaatctcagcactttagaaggcttgaaagattggcagtatgtacactctactttggtatatgcttgaaaatgtccatgataaaagttaaaacattcagattcccagartctgaaaaaaaacatataatgcctcttcttcgaaaagggatgcagttatctactttcatgtaatttacatatatccttgaaccctcccagaagtcccagattaagcttaaaatatacatttatatttcattcttttgaattttgcagattgagaaaccaagatgtcagaagatgaaggattggtccaaaataagaaagctagtaagtggtttcatgaggacagaaatcttttgacttttagtccaaactcttatcttattaggcctctttatccctagattgggagttcacttgggaag

SEQ ID NO: 199 - DNA, 599 bp, Homo sapiens
tttagaaaactgcagcatcaaatagttctatccatctcgttgcagtttcaaattttccctccatccaaagggtttgaggtatctgagaaattaaagcatgggttaaaaactgagatatctggtttaaaaaataaaaatcagagatgattcgaagggcaaagacggaaggaagtcatttctccctcccccagcatagcagatacgggagggcacaggctcctgcccccacatcagaactctgccatgctcaggcccccagagtggaaactggcggagaagaagaaaagtctaccaactgcwctttgggatcagaagaggctcattccaagggcgaccagagaggagaagggagagcagcatgcactgagcgccacagggcatctgccatgggccacgccctcccgctgggtgttttgtataggcacatctcactgactcctcagacaactccatgaggtggcagtcatgaccccccttcactgtgatgaaagaggctccgagcaatgaagtcactgtccaagttcacagaggcagttggtgatagaggaagacttgagttctgagcgagtgtcattcggattgagccgttactaggtgcc

SEQ ID NO: 200 - DNA, 599 bp, Homo sapiens
tgatgcaccttaaggtttgagaaccgctgctatttaatataaaggcttcctgtggtccctcaccctctgagtccagctagagctatgaaaaccaaattagcttccagagttttgtcgggggggttttaactgacaacatatagttgtacgaatttatgggtgattttatgtgatgttatgatttctgaatactatgtagaataattaaatctagctagttaacatttccatcacttcaaatattaaacattgtttgtggtgggaacgattgaaatttactctcttagcaattttggtatrtacaatactctattattaactatattcaccacactgtgctagtactcaagaaaagaaaagaaaaaaacatattcctcctgagattttgtacccctttgaccattatctccccattctcccaccctgccggcatcggtaaccacccttctactctctgcttctatgagcgtaattgttttagagtccacatataagtgagaacacatgcggtatttgtctttctgtgcctagctcattccacttagcatcagattctccaattccatccctactactgtcataaatgacagaatttcctt

SEQ ID NO: 201 - DNA, 599 bp, Homo sapiens
acgcggcatgaaggtcgaagccctgcccccttgttgccttgactctgccctcattaccccaagtgcctcctaatggatctgtccttattcttccttgttctgtctccctcatgccctggagctcctctctttgggaccttcctctcccaagtcaccaccctctctccccttctaccactcagccattccccagtttccacagttcctcaaacattaccccactccccactccccagtttcctccccctccccatttgtccccctcacctgggggtccccgaagtagatctgcatgaccarcgccacggtgacaccggtagcgaaggtgagacaggccgtgacgatgaccgtgagcccatcctggcggcaggagcactcggccgctgcggcggagaacgggtctttgcgcgtctcgcgtagcggcgacccgtcttggctgcccatctccgacgacgacgatggcagccgctgcagccgtgcagacttcaggaaggagtccgggtctgcgggcaggcagccggggtcggtctgggcatctcctatctggccctgcccaccctccaggtgggactattcagcgctttccctgccccgcccct

SEQ ID NO: 202 - DNA, 599 bp, Homo sapiens
gacggggtttcaccatgttggccaggctggtctcgaactcctgacctcaagtgatctgccctccttggcctcacaaagtgctgggattacaggtgtgagccaccgtgcctgacctctcaatcatcactttcatgggaccatggaggcccagaggtctccttgcctaggatcacacgcacagatccggcagggatggagaggggctgcgctctggctgggtccaaggcctctgccagcactctgagccagcccacagctgcctcccacttgtgtctccccgttctgtgtccaccttccagrcccaatcccaatcccggcccctgctccatctttgttgggtaactctgagcagtgggggatcatcaggcagttctgtgttagctcccaggaacagggtaactgggttgctcctgtctccacgagggccctaagaaagggattccaggcaagggctttgggctccttaaattccagtggttcccaggatggtgttggtgtcctgtgtttggggctctgtggttgccatcctgcaccccatggtctccaaacagacctgagcctgtctacatgcacttatcctcaggccatcattcattcat

SEQ ID NO: 203 - DNA, 599 bp, Homo sapiens
atcacttgaggtcaggagtttgagaccagcctggccaacatggtgaaaccccggctccactaaaaatacaaaaattagctgggcatggtgatgtgtgcctctaattccagctacttgggaggctgaggcatgacagtcacttgaacccacaaggtggaggttgcagtgagccgagatcgcgccacagcactccagcctgggtgacagagtgagactctgtctcaaaagaaaaaaaaaaaaaaaggaagatatgtggggctctactagcctggggagaagcttgggttttaccctaaggaytatgggaggtcactactcgagaggctatttttgtttgtttttgagatggagtttcgctcttgttgcccaggctggagtgcaatggcacaatcctggctcactgcaccctccgcttcctgggttcaagcgactctcttgcctcagcctctccagtagctgtgattacaggcacatgccaccatgcctgagtaattttgtatttttagtagaaacagggtttcagcatattggccaggctggtctcgaactcctgaccccaggtgatctacctgcctcagcctcccaaagtgctgggatta

SEQ ID NO: 204 - DNA, 599 bp, Homo sapiens
cagccagctctggagcctgtgctcttaacggttccatcaatgcacccactgcagatggggaaactgagttccaaggtgggactggcccagtggcacagaagtgaggggtgacatcggttgagcttggcaggacttatatcaatgacgcctgccttagagtccccactgcctccagggaggaaggaggaggcatctccttggggagtcaggggcaaaacacgtttattcccagtttgacttgaaagcaggccttggtggagacagaaatgtctcctgcctctcagaattcacagacgggcrgggttggagcagggaggaggtggcattgattaaacatggaggtaggacctgggttggggctatggggtgggaagatagggagcactttgtccagtggagagcgaaagatgagttaattaattcattcacttgttcatttgttacactggcatctatgcctgtgctggcctaggagtggggcacatagctggttcgcctggagaaacaacacttcactatgggccccagggatctctggatgggccagaggaactgtaggggctgggggaagcttcctgcagctgcttacctttcaccct

SEQ ID NO: 205 - DNA, 599 bp, Homo sapiens
aaaccctgaggcctggccactggtctttgtcttcacttttcccttttcttaactcaccaagaagtcgagcctcaggggacagaaaattagcagatctgcgggagggccttggacagggtgaggaagactaaggcctctccttttccagagagtgaatgagaccatggccacactcctgactgtgggcagaggcctccccagaactgcaaaacttggacatcccagtgctgacccaaaaccccaaatgtgccacctccccgggtatggtgatcatctggcttgtggtggcctcctgggagygggccaaggacattctgcagagcgccagggaattcccgctagtgcacttaccacccactcaagagccgatggcagccaacaggcaatgctgcgccattaaggtgtttgtcaagaagctaagctgggcattgtgggcgatcctgagggccctttttagattggctgtaagtgaagaaaaaaaacagcatttggaggcaaactagagctagtgcagagggtgtagcatgacagaaaaccctctgagaggcagagatgcttgcctccatgtctggcttccttaccaacttgctctgtgattt

SEQ ID NO: 206 - DNA, 599 bp, Homo sapiens
ttgaaatgtcctttctagtttccccattttacaggggtgcagactgaggcactgagaaagagtcattgtcacaagtttatgctgctagcaggctgggcagaacttacccctactcagtgtctgctttcatcaagggaaactaaagggggctgctgtttacggagctccgaggctccagggacattccacacatttcctatgtaactctcaaaaccctcctgggaaagtgtaattatgcccattttgcaggtggccagacggtggccagggagaagtgattgttgaggctaaaagggcagrtgaatgtgggaacgcaggagtgtgtgactcttggagtccctatttcccattccaggaacccctccagcccgcacaggaggcaatgaggtggtctgggctgaagctacttcctcatgcccctcatcgccctccagacttgctgccgtgtgggacccaggatgataaggacgtaaatgccactcgctgagcccagctgtgtgcaaggccctgttcccagagctttgcaggtcaggacagttccaatcacaatcttacaaccaaggaaacagggagaactggcagcagaagtcaagcccagc

SEQ ID NO: 207 - DNA, 599 bp, Homo sapiens
tgactcctccccagcgcacagctctgcctggcttggtcccttttcactgcagcctcacgaacccttttacctccttgctctctggcctcctctctacctctctaatgccaacctcttccctgcctcctggcctttgcccatgccctccccactgcctggattactctcccaccctcttttcatctggccaagtcctacatacccttcagtcctcagtccaaacaccatttcccacaccagcttacacccctggcacgtgcacccagagcccccagttcttcatcaggccagtcatcgtgrtcattgttatctactaacatcacctgaatttctcaggagcttactgttttttcataaagccaacttttaaattcaatttattttgggtgggaggcagggaaggaaaaagggattttcctacaaaaaggcctctacttggagagagagagagagatgagcctgggccttttatatcctctcctggggatgggacagttatgattggcagcggtgatgcccaatctcgtcatctaaagtgagtttcataggctacgagccactccccacccactccgaggcattgcagagacacctggtaa

SEQ ID NO: 208 - DNA, 599 bp, Homo sapiens
agggtcctgagatgactcttggctggggctgcctagtgctacctgctggccagggtggtggcgcccagccacatctgcatctgcactttctcctccacagggaagcagtgccagcatgtggatggcctggtgtgtggctgcgctgtctgtggtggctgtgtgtggcaccagccacgagacaaacacggtcctcagggtgacgaaagatgtgttgagcaatggtgagtccagccccaaaggggtgagggttggcatggggtgagcagggcagctctgccaactctgaagtacccaggaacytcctcctgctagcagagttcacatcgggaggctgggtgaattctactctctgggccttagtttctcctttttcttttgactaccatttcctgagcacctactatgtgccaagcaccatactggtgactttatgctcattctttagaggcaatagagaacaatgattaagagccctggatcagccttcgtggggttcaaatcctgactgtgtgaccttaagttcatgacttgagttccctgagcctcagtttctttatctgtaaaatgggtgtaattatagtctctatctcagagaactg

SEQ ID NO: 209 - DNA, 599 bp, Homo sapiens
aatttgtatggaacaaaaagtccaaatagtcaaagcaatcctaagcaaaaagaacaaagctagaggcatcacaccacctgaatttaaaatatactgcaaggctctaataaccaaaacagcatggtattgatataaaaacacagaccaagggaacaaaaagagaacccagaaataaatccacgtacagccaattgatttttgacaaaggtaccaagcacatacattggggaaaaatgttgttgggaaaactggatatccatatccagaagaatgaaactaaactcctgtctctcaccataygcaaaaaatcaactcaaaatgtatagggaaaagaaagagcgatcagactgttactgtgtctatctagaaaaagtaagacaaaagaaactccattttgatctgtactaagaaaaattgttctgctttgagatgctgttaatctgtaactcttgtcccaaccctgtgctcacaaaaacatgtgctgtattgactcaagttttaagggatttagggctgtgcaggatgtgctttgttaaaaatgtttttgcagccaggcgcagtggctcatgcctgtaatcccagcactttgggaggccgag

SEQ ID NO: 210 - DNA, 599 bp, Homo sapiens
ttgagtccgagggaagcatcagggacctccgaaacagtggctatcgcagtgccgagaatgcatatggaggccacaggggcctcgggcgatacagggcagcacctgtgggcaggcttcaccggcgagagctgcagcctggagaaatcccacctggagttgccactggggcggtgggcccaggtggtttgctgggcactggaggcatgctggcagctgatggcatcctcgcaggccaaggtggcctgctcggcggaggtggtctccttggtgatggaggacttcttggaggagggggtgtcytgggcgtgctcggcgagggtggcatcctcagcactgtgcaaggcatcacggggtaaggaggggacgggttctccccagaaagcccccatacacctccaaatgggggtgatcactccctgaagctggggttgccggagggaaggtcttcagagccccacacacttcagggtttggcctcaggattggaagttttaagacccccaagttacatggtttgggtcttggcatgaaacagtcctaagtttgagacccagttcagcttttcatgtttgcgtgtgactttgggccattgatatcat

SEQ ID NO: 211 - DNA, 599 bp, Homo sapiens
agagtcccttaatcccacatatttctatggtgttaagtttatggggttttcttctcttggttcatcacaggaacagtggctattttttgtaagtccctattgtgtaccaagcactaagtactactgtcccctcttcgtatgaagatactgaggttccatgagataaagtgacttgccccaggtcacccatctagtaggtggtagggctgagtatctttgattccaaagcccatgctcattccatttgtcactctgtctgtctcccagtcacctgtcatacctggggggaaggggtaatgrtggttattacctttattttactaatttatctatttcaacttattatgcaatatgtagaactttttttttttttttttgagacggagtctcgctctgtcacctaggctggagtgcagtggcgcgatttcggctcactgcaagctccgcctcccaggttcacgccgttctcctgcctcagcctcccgagtagctgggactacaggagctcaccaccatgcccggctaatttttttggattttttttttgtagacacggtgtttcactgtgctagccaggatggtctcgatctcctgacctc

SEQ ID NO: 212 - DNA, 599 bp, Homo sapiens
ttaaataaagagtataggggctaaattatttttaaagggaagcaaaataggatataaaccactaaaccacggatatagtatgaccctcgatttataggtagggaaaaccatgcctgtaaagaagtaattcagccacaggtcacacagagagagtcaaacagagctgggatgggaacgcctcccctgtgcccaccccagccccagtccagttctctgcccgccccacccaagtggcacccaagtgccttctggttcaagacgagaactggacaaccagccagaggcagagatttcctctcygggcctaaaggtcaaacaactccagagcagcctatgcgtcaccaaactgtgaacaaaggtgtcgacacttccaagaaccacagggtctagcaactgtgtctgccacagacaagaaacttgcaatctggcaggcaccagcgggagagcaagtcacaaccaggaagggctctaagcaatatttcagaaaaagtcaggctgtaataaaaagaagaggagtgacaaaatcctgtttcttgacctttttttataaaatgaccaatgaccaaaaataattaaattataaaggaggagcagctagg

SEQ ID NO: 213 - DNA, 599 bp, Homo sapiens
gggaggcagaggttgcagtaagccaagattgcaccactacactcccgcctgggcgacagagtgagactccgcctcaaaaaaaaaaaaaaaaaaaaaaaaaagaagacggcacagagagcacaatactgtagaaatgagtgcaaatatatcagtaattacagtaaatgtaaatggattaaatgtgccagttaaagattgtcagaatggataaacaaaatataatacactatttgcaaaagatgtatctaatcataaaactacagaaagatggaaaggatagaaaaagatacatgggaaaakgtcaaccaaaagaaagctggtatcacttatattcctatcagaaataaaggtataagaatgaccttcatttacagatgacaaactgggctcagaagctgggagacatagcagcaagtacagcctcaaactcaggccggattcctaaaccaggattttagaccatgttgtccctctgctctcttctcttttttaatcttttatggatcatatgtctggaatataagtttcattgcagaaaacatacaaaataatggggaaaaatagaactctgccatccagaggtgacagctgttaagtgg

SEQ ID NO: 214 - DNA, 599 bp, Homo sapiens
tgtctccacggagcttgcaggttgatagggtacatgggcattgataagaagcctaaacataaccatgcagtttcaacagtggcaagaagttcatggagaagtaagaatctctataacggggcagctcacctacctagggaggtcaagggaggcttcctggaggaagaggaccttaagttgaaatctgaagaatgtataggagggaattaagcaggaggaacctatatgtataggagggaaccaggcaagaggggcagggatgagcagagagagggaatggcatcttgacatgcagaaaakattcaaaggaaatatacggaatgcacacagtgggcctctgggcattgggattacaggtcatggcatttttgccttaatttctctgttttccacattctgtccttgaactgcaggtgtctagtgtctttagagtcagaaggaacagacggggagcagagaaagggggtgtaggggagggcgctcgggtgagtgggtggtagttggggtggccggccttctcaccactctgtgtcccgagcaggctgcgtatcgtggagctgaccctccctcgggtgtccgtgcggctcctgcccggcgtg

SEQ ID NO: 215 - DNA, 599 bp, Homo sapiens
cactctcttctgttgagtcgtggtgaagcagaaaccccaccgccttcctccatgggttacgataagattcatagcctctcggtggcctctaaaaatcttcctcgaacttccagctcctttatagaccacattcccagcagaagtgtgagctcgcttgggaccacctggtgtcacagtccaggctgctgctggtcctgctcctctacgtggcaatcggcccagagaatatgccttggggggtctcagcagaaactaagatgaaggtaattcaacttcaactccctagttcacaagaaataygttagctgttttccctaaaaaaggctaggtgaaatagggaatttcatgattttttgtggggggcagggggggtacacaatctccctcttttcacccaggctggagtgcagtgagtggcctgatctcagctcactgcaacctctgcctcccaggttcaagcaattctgcctcagcctcccaaggagctgggattgcaggtgtgcgccaccacacctgggtaatttttctgtatttttagtagagacagggtttcaccatgttgctaaagctgggatccaactcctgggctcaagcaattc

SEQ ID NO: 216 - DNA, 599 bp, Homo sapiens
gctgcctagtgctacctgctggccagggtggtggcgcccagccacatctgcatctgcactttctcctccacagggaagcagtgccagcatgtggatggcctggtgtgtggctgcgctgtctgtggtggctgtgtgtggcaccagccacgagacaaacacggtcctcagggtgacgaaagatgtgttgagcaatggtgagtccagccccaaaggggtgagggttggcatggggtgagcagggcagctctgccaactctgaagtacccaggaacctcctcctgctagcagagttcacatcgrgaggctgggtgaattctactctctgggccttagtttctcctttttcttttgactaccatttcctgagcacctactatgtgccaagcaccatactggtgactttatgctcattctttagaggcaatagagaacaatgattaagagccctggatcagccttcgtggggttcaaatcctgactgtgtgaccttaagttcatgacttgagttccctgagcctcagtttctttatctgtaaaatgggtgtaattatagtctctatctcagagaactgatatgagaatgaaatgaaatgaccttt

SEQ ID NO: 217 - DNA, 599 bp, Homo sapiens
ttctttgaggcagaaattgggcataagacaatatgaggggtggtctgctcccttatttagggatattgagtttcccaaaccagttacttgttgattgtataagacacaactcacatttatttgtccaaaacttccactcttttttttttaaaacctcttgcattttccgatgctgtcactcttgaggtttcctgaaatcccgtcagttctaggaattcatgacacttctctcctcccaatttgggatgtggttacacaatcatggtcccttcagtctccatctcaaaccggaagctttayccctgtcagccctgtagaatgtcttgggtaagaatgaataccctggttgggcgcggtggctcacgcctgtaatcccaacactttgggaggttgaggtgcgtggatcacctgaggttaagagatagagaccaacctggccaacatggtgaaaccccgtctttactaaaaatacaaaaattagccaggggtggtggcgggtgcctgtaaatcccggctactcggaaggctgaggccggagaatcgcttgaaaccggaaggcggaggttgcagtgagcgaggatcgcgccactccactccag

SEQ ID NO: 218 - DNA, 599 bp, Homo sapiens
ctgccctagatctgatccccctaaaactaaatgaggcaaagtaatgcctctgcctgtcccacttacagtcacaacagagaaaccacagtgaaaatcgcaggtattctctgacccacttaatgcacctgggagggttggctgatgtagctcaattgtaattaaatacatcacctatagtctggcattctcctctaccattgggaggcagtataagattgcagcttaaagcacaggcctttcaccaattttcacctctaggaatttaattcacagtgaacagctgtttgagagtgttcatgrcagtattgcttagaacagcaagaggctggaaagaacccaactgtccacccattaatgggtgattaaactgtgttataaacagtgcaaaactatgagttatcaaaaagattgttatcaagacttctatgtgctgatggaaagacattcgaattgtttttaatgaaaaagtcagggtggtttagtatgtttccatctgtttcaaaaagacatgctgacagaaagcagacagcaattgttagggtcagtggtggtgcagggatgaactataaagggccacaagggaactttttaaagtgaca

SEQ ID NO: 219 - DNA, 599 bp, Homo sapiens
aattctagggtatgggagatatttattgtatataatcttttaccacttgaatttgccatttgtatttactttgtttcaacttaaaaaaataaaaaccatcacaagttttgagtttaatttctcccgaccttagcacattcgtgtgtaaaacagggataatggcacctaactcgtaggggtattaagatactgagctgagatctgaatgacaagcagcagccagtgatgggaggaaaatggggagtgtatttcaggcacggggcacaccaactacaaagacttaagttttaggagtgtcasactccttcctggatctgaatgcccacctacataatgaaaggggtgggttttctaattcttggagcccttccacggtatctcgttactccagaaattgcctatgtccccacagctgatgcctgttactgctggggctgagctcagcgatccagcccacccggctaatccaggactttttttttcactgcgagaggctaagtggatcctggaggtgatagtaagaattgcggggagcgcggctctagctcagctgggagctttctggtgccggtgaggagaccatgaagccaccagcccca

SEQ ID NO: 220 - DNA, 599 bp, Homo sapiens
gcaacctccgcctcctgggttcaagcaattctcccgcctcagcctcccaagtagctgagattataggcgccagccaccatgcccaactaatttttttttttttttttttagtagagacagggtttcaccatgttggccaggctggtcgtgaactgctgaccttgtgatccgcccacctcggcctccccaccacgcccagcctactctgacttttctttaccatggaacccccctccttttccttttcataattaggaagggtcctcaacgttgtttcctaagacactttggaaatgatgrtctggttcccttccaaaactagaaaggggctcttgattgcctttgctttgtcaccacattgcaaggttccttcagctgagctgtgtttggtgagagacagtagagacagaagaggttgatgctactgtgtctgagtaacatagggcaacccattatttttcatgggttttcaatccaatttagtcagttatctcaactggactcttacaggggctgcatcagtcctgagaatctgcttcaagaatgaagttaaaaacgggttgaaatcaccagtctatttgtcaacctgtcaccaaact

SEQ ID NO: 221 - DNA, 599 bp, Homo sapiens
cccactactctgcgggtatgaccccagatgacgaggatgtatttctctgcgggaagtgtaagaagcaattcaactcgctgccagcgtttatgacccacaagcgggaacagtgccaggggaatgcccccgccctggccacagtctcactggccaccaacagcatctacccaccttcggcagcacccacagcggtccagcaggccccaactcctgccaatcgccaggtatttgttcatttattcattcaacatgtcttttttgagggcctaacatgctcaggcactgtgctgggcactgggkctatagcaatggataagacagaccaagtccctgcccttgtggagtggggcgagatggatggtgcacatgtactgttttcagcatgttaaatgcggagagataggggatgccagacggggatgggaggttttgccatattgtctatcgtgcttggggaagagctctgtgaagaggtgaccaggaagaatactatatagcaggtaaaaggttttgtatgaaaaatcaaattgtagaataataagtagataatatattattatttttaaaatctgaaacaatagtactatataatttccgca

SEQ ID NO: 222 - DNA, 599 bp, Homo sapiens
gtaaacacagtagcaaatcagaagttctaaattttgacatttcagttacccacacatttggataatgttttatattttgaaacctgattatacatagtcgagtttactaaatcccataacaaccctttagataatcagtcacatattaagggtgacagaaaagaattctagaaagactgtgttacacaataagggaaaggacaagaagcaaaccaaagtcataagactccaaggacagggacctttctacactgttagagaaaacacagaaactacatgttctgatattagggttgaacrtgtaaataccaacactgcagtaatgatgtttagccagcaattcacagatgagcaccctgtgagcagatgaaaatagcatagctagagacggggcagcatctgtgtcattattacatactcactttgcaaggtacactgcatctgtgctggctttaaggtggttttattaatacactaggtgatagatatataaagcataaaattcataaaatgcaaaaaaaaagaaaagtcacaccagtggaaaaaaaccgaaaacctctatgtacacggtttatatatccacccaaatgtttacttat

SEQ ID NO: 223 - DNA, 599 bp, Homo sapiens
acagtaacaaatgttatttgcaatatgtatatagattttttaaatgaggacttcgagaatatctgtgaatcatcccaccattattgtatctcatttatgaaacagaatcagtgaaaagcccagagaaaaagaaggtgaatccatgtaaaagctgaaagaaaaaagggagagaaatatagtaagaaccaagtgaataggggctagataaataaaatcttacagagggagggttaggctatttggcaaaaagaagcttaagagagtgttgaagagaaactaggtaaagataactgggaaaaygaaccatgaaattcaggaggtattgctagaattctgatttagattccttttaacatacttcaggagataggagtaaattataaaagttgttctcaccctacctgttgttttgcacaacactctaatctaggaaaaaaccaagtaagtctaaaatatcaatagagtttccctaaattatttataattgttgagctgtcactacatgccatataggtgagatagtagataagcaacaatgtgcgtggactgcaatagcacttcacgccttgctccaatccaagagcgtaaaaaaactagat

SEQ ID NO: 224 - DNA, 599 bp, Homo sapiens
gcgtggactgcaatagcacttcacgccttgctccaatccaagagcgtaaaaaaactagataatggcaagacatattataaagctacagtaatcaagataatatggtattggtaaaaaatagacagatcaatggaacagaaaagaaagcccagaaacatacccatataaatattgtcaaataatttttgacaaaggagcaaaggcactacaatatagagaagacagtcttttcaacacaggttgttggaactactggacatctacatgcatagaaaatgaatatagatacagattttagayccatcacaaaaaaattaagtcaatatgggtcataaacctaaatgtaaaatgctaaacaataagactcctagaagatgacataaaagaaaatctgtagaaccacggctttgttaagtactttttggatgctacaccaaaggcaccatacatgaaaggagtgattgataatctggatttgattaaaattaaaaacatttgttctgtaaatggcatcatgaaaagaataaaaagacaagctgcagagtgggaaaaataattgcaaaacacatatcagataagtgactattatctaaaatata

SEQ ID NO: 225 - DNA, 599 bp, Homo sapiens
tattggaagaagataccatctaggactttcatatccagaaagtcaatgcctgccttcaaagcttcaaaggaaaggctgaccctcttgtcagggtctaatgcagtggtgatgctaatttgaagccaaagcttatttgccattctaaaaattttagcgcctttaagaattatgctgaatctactctccctgtgctctctaagcctggatgacagcacatctgtttacagtggtttactgaatatattaaatccattgttgagacctgctcagaaaaataatatttctgcatctcatcaacartatacttggttatccaagagctctgatagagatgtacaaggaggtgaatgctgttttcatgcctgctaacacagcgtcccttctgtagctcacagatcaaggagtaatttttgattttcaagtcttattatttgagaaaaaatttgtaaggctatatagctgccatagatagtgattcctctgatggatctaggcaaagtaagtttaaaacctctggaaagagtttagcattccacatggcattaaaaacatccatgattataagaggaggtcaaaatatcaacattaacatatatttg

SEQ ID NO: 226 - DNA, 599 bp, Homo sapiens
tatattgattagttttaaaatggattttttattattttaaagttctaaactacatttattgatatatcttaaatttattttattataaaatttaacatttcatttaaatgtaaacttgcatttctggccctttattggtcctaatgcattacgctttttagtattgctggattcaatagtgccatattttcaaatattttgtaactgttttcataaaagatattaatctgcaattatcttttctcataatatctttgttagattttggtatcagaataatgctggcctcattgaataatytggaaagccatccctccttttctttcttcctttttttttttttttttttaaataaacggcatctcactctgtcacctaggctggagtgcagtggcacgatcatggcttactgctgcctagacctcctgggctcaagcagtcctccccctccagcctcccaggcagctgggattataggcatgtgccaccatacccagctaattatatatatatattcacacacacacatatatatacacatattggatcctgtctccctaggaaactgcatagtagagacaaggtatcactatgtttcc

SEQ ID NO: 227 - DNA, 599 bp, Homo sapiens
attagaccatttgctgtgcttccacaggtccctgagcctttaatcacttttcttcagtatttttcactctctattcttcactttgaacaatgtctattgatgttttagagttcactcatctctaatagtgtcaacagcactatttattcctgcagggatttttaaattctgttatttaacccatctgtaatttattttgctttcatagctagagaaaaaaacaatcttttaaatattttatttttctagatggctggttatcttaaggcaatttattgaattagacaatagttttcaaawagagctccacaaaaatatagatgacactccttttctctagagtctacgcattgcttccatgatttcaggtgtgcatttctgatcatgtcatttccccaaaattaacattattgtaaacaaaacccatttgcataatttggtcattaaatgctttttatctgggagagtaaatagtaattgcagtccccaaggtaaagtcttatatgggtaaccggcaaaaatggtgtcctttgtttcttctataaacagagaaattgcatatagctaaaaagagtaagtttcttaagaaactcaggcta

SEQ ID NO: 228 - DNA, 599 bp, Homo sapiens
tctattcttcactttgaacaatgtctattgatgttttagagttcactcatctctaatagtgtcaacagcactatttattcctgcagggatttttaaattctgttatttaacccatctgtaatttattttgctttcatagctagagaaaaaaacaatcttttaaatattttatttttctagatggctggttatcttaaggcaatttattgaattagacaatagttttcaaaaagagctccacaaaaatatagatgacactccttttctctagagtctacgcattgcttccatgatttcagrtgtgcatttctgatcatgtcatttccccaaaattaacattattgtaaacaaaacccatttgcataatttggtcattaaatgctttttatctgggagagtaaatagtaattgcagtccccaaggtaaagtcttatatgggtaaccggcaaaaatggtgtcctttgtttcttctataaacagagaaattgcatatagctaaaaagagtaagtttcttaagaaactcaggctaattttttatgcagctcaattttggcaaaatgtaatatatcttaataagtgttctttttatcagccattt

SEQ ID NO: 229 - DNA, 599 bp, Homo sapiens
taccctagggctcctcacactgtttctcacagggtttatttttcattggcttagtagaatatctgcctaaactgctcagaactcacatgaaagtttgtgaaatttaatactcatgtggttaccttgaccaatggaagatggaaactgccgcataaatgctttctcttctctcccctgagggagacattttgggggtacacttcagagatttcctcagaaaggcacactggatcaagaactagtcacccatgctgttggacaacttgataatgcatctccaaatttgcttttttctttgtyccctgtttctctcttacctgtacctcattcctaatctgtggaaacacattgttacataaaatatttatatattatttttcatctcaagctctgctttgaaaggaaaatccagcctaagtaaatagaagaaaaactcagtggcggtaccctttaatttatcgccaccacatactctaggaaatacagcacaaaccatgttgtggaatatgagtccattagattagcatcttgcctctaaaaaaaagagcaagaagaacatggggaaagaagcaaattattcctcttcatccataatgact

SEQ ID NO: 230 - DNA, 599 bp, Homo sapiens
tggatcaagaactagtcacccatgctgttggacaacttgataatgcatctccaaatttgcttttttctttgttccctgtttctctcttacctgtacctcattcctaatctgtggaaacacattgttacataaaatatttatatattatttttcatctcaagctctgctttgaaaggaaaatccagcctaagtaaatagaagaaaaactcagtggcggtaccctttaatttatcgccaccacatactctaggaaatacagcacaaaccatgttgtggaatatgagtccattagattagcaycttgcctctaaaaaaaagagcaagaagaacatggggaaagaagcaaattattcctcttcatccataatgactgtcaaaaaccctaaagtttggaggaagtaaaaatccacttcaaaaaaagtcaaagaaatgaatcattaaggtatgtaatattaactgtaagtactgcaaaaatgttcaggccaaagggacactgacaggattcttcctggagaaggctttatggcaaaggggttgggtgtgtcttgaaaaatggtttacatgaacagagactacaagttattctagaattggagttt

SEQ ID NO: 231 - DNA, 599 bp, Homo sapiens
gcctaagtaaatagaagaaaaactcagtggcggtaccctttaatttatcgccaccacatactctaggaaatacagcacaaaccatgttgtggaatatgagtccattagattagcatcttgcctctaaaaaaaagagcaagaagaacatggggaaagaagcaaattattcctcttcatccataatgactgtcaaaaaccctaaagtttggaggaagtaaaaatccacttcaaaaaaagtcaaagaaatgaatcattaaggtatgtaatattaactgtaagtactgcaaaaatgttcaggcmaaagggacactgacaggattcttcctggagaaggctttatggcaaaggggttgggtgtgtcttgaaaaatggtttacatgaacagagactacaagttattctagaattggagtttagaatgagcaaatggaagcattttatgcatagttgaagaaatgtgcatgacaggtgaatggtgggattgagtcagagaagtagggtagcagtaaagttgatctgtgcttcattcctgtccttttgtaagcaatctgcccttcctcccacatccctccaccatctatgaacatgcatgttttctg

SEQ ID NO: 232 - DNA, 599 bp, Homo sapiens
tcaaaaatattaggaagattcctgagtaaagaaagcaaaaatttcatttagacgtattctttccttttctcctaaatcttatctcttcccatttctcttaatcatatctcatgaagcgtaatcttatgaaggtatagtgaaaggagaaaggacaaagaaaatgtgaatgagttgggcattattgaagggggcctacctaacacaaaaatttttgcaaaactactccttaaactagacttgaaatattgtattggcacatgttttatgtcagaagctatccattggtcattaatctatgckaaaattttgcaatctagtccttcttctgtgttctctgtattgccccccgtaacctctacagtcatttagaaagttataggagagttctgttagctattatcatggacaattacttgattgactaaatataaaagaaaattaaacttctttctcaagaattctaaaattatatttagaatttgatttaataaacatttgctgagtaccagccatctttatgactctaaactgaaaatgcagaaaataaataggtacattgatatagagatgactgatgagaagcatgatttcaaattata

SEQ ID NO: 233 - DNA, 599 bp, Homo sapiens
catggttcattgactcttctaggtagtggagattataaaagatattttctttttaagattatcatgataatgttataagaaaatatacatcaagtgtctaacaagaacagaagtctatttctttaaaattctttttcttcctcttccctatacaagtagagtaagtgaaatgctagaggtaaaaggttgtgaacattccttttttgttgttgttttttttaacagtttcagttctccttagaaactaagtgattctgaagacaccaacaattggattgccaggatatttacagtggaagytaacccaccatggacagttgttaatgcgtttgacttgctaaagcactttaagaatctaaaacaaaaattgacaagtaaagagacaaaaagatactttctaaggctatattaaaatacaaaagcacatccttatactaatatggcaaagtacaattttctaggagctgtaataggtagaaaattagatgataaacaaccggcagtgcaatatatttaatcaagggcatcacaagatttcagaacgtaaaaagtgacctctagaagtatagattagtgcaattttatctaggaaaaagtct

SEQ ID NO: 234 - DNA, 599 bp, Homo sapiens
tcattcaacccagaaacctctacattttgcccttactacagctgtacatatttggtggaaatagtatatccctgcaactcacattagagacatattctaaaaccatagtgaatgattttgagctagccagtaaacaaattgaagtttgttaaaattcattctggtgttctaaatgaatctcctcccacctcctccatctacagtaatcatgtggattaaacagcaagaatagttttgttcaatacgttcaccctgtggcaatgtagctgattacattagaaataacctactaatgaaackactacacaaattatcatcctgaactatttaggccagttcacaactggatatgccctttatgctggctaattacatgcacataatgccaagataaaggagactgtcattctaatacaatgttggaagatggcttaatcttactttggactggtattttcgtcaataaatgagaaacaatgttggcaatgatgtcaagatgggcaactaaaaatgtttgaatcctgaaagatctcatgtatggaccaaaatagacaaaatgaaaaacctataaagcagtagacaacagataaatcataatt

SEQ ID NO: 235 - DNA, 599 bp, Homo sapiens
gaaaaactatcttcaatgaggactgtgccataaagaccctccaaatctccattttggttaaaatattcactactgggctccatctctctcttttttgtttgtttctctctctccctcttttctccctctgtagacacacccatttgcatcagctttccaggaaatgtgtataactattacataacttcaactgcattcattcattttaatttgctatcaaacaaaatcatgtttcaaatgtttattatttgtatttacctcttattttaaattttgtctttacagtgaacatgataacakttattctatccttaaatgttaatgccctgaaaattacctagtcacctctgaccacagagacttgtcaatgatttttctttttggtctgaactgagaagtgattattttcttctaaatattgaaaatataaatttttttcgagtaaaaaaatttaataagatttttgaactaagataattatctctcaatatgcataacattatttgctcctaattgttttagtgattgggaggagtttcatcctactttgcaaaccagttatgtatttctctgttcagttttttataggtatgcatcct

SEQ ID NO: 236 - DNA, 599 bp, Homo sapiens
aggtaaattctgaatggtctaatcggcatggatttagccagtagattggaatgcattaagtatcttttaggtctagctggcatgtcttactgatagatggagatggaatcagctttactcaaagcacaaacattgaagccacagtgggagaaaaataattaactgggtactactggattagctgggtactctcactaaaggaagaaagaatgtgtgctgaatggccctgcaaaaaagtaaatactattcagctgcaaatcttatttttacaaaagtatcattttaaatagaaacatttayatttgagccatactcttcctttgtatacctcctcttgtcccaacacatggtggtcatggtggcagagtgtttctgcttttggaaaaggctgtattacagatgtagatgaatctgaaaattagactgaggaccccctaaggaaatggaaccttttgatctctggccttgattgacagctatttgagtgaacaaaaacctggaagtatttctctaaagtttcagattagatgctaatttttgttgttgttgttgttgttgttgtttgtttttgttttttaacatggacaagctttgcctct

SEQ ID NO: 237 - DNA, 599 bp, Homo sapiens
tcaaagcacaaacattgaagccacagtgggagaaaaataattaactgggtactactggattagctgggtactctcactaaaggaagaaagaatgtgtgctgaatggccctgcaaaaaagtaaatactattcagctgcaaatcttatttttacaaaagtatcattttaaatagaaacatttacatttgagccatactcttcctttgtatacctcctcttgtcccaacacatggtggtcatggtggcagagtgtttctgcttttggaaaaggctgtattacagatgtagatgaatctgaaarttagactgaggaccccctaaggaaatggaaccttttgatctctggccttgattgacagctatttgagtgaacaaaaacctggaagtatttctctaaagtttcagattagatgctaatttttgttgttgttgttgttgttgttgtttgtttttgttttttaacatggacaagctttgcctcttccttaatggatgacattttcctaaaagatgtattgaatttaaaatgccattgattgtaaaacctaacatctcttgtacaactgggacgagaaaagtagccagttaaattatggaaca

SEQ ID NO: 238 - DNA, 599 bp, Homo sapiens
caaaggcaatttcaaagactaggcatactacttccccagcttcttctatcttaccctaagatgatttccaaaacaacccacttaattgtgtggaacacttcactttcctttttttcctcaattttaggttttgggttctctagaattttatccccaggagaaatataagaaatccataatttaaaagagaagaaaataaatgtgcacttagtgtaactataaaatatttgtgttgaggttggattacatttagagtattttaaaagaaaagtatcttccactttaaatatatgctatttkgtttaatgctgaaaaaaattcttaaaaattaattgatttatgacttacttacttcgagtataattagtgtaattttattaaatttaacactcgcttttaatccccctattccaacattagatatcaggtaaacccactctcatgagaatattagttaccttgttctaccttcattattgtctttgtggagtgttataattatttttcttatcagttcattctgcacagttctggtatcacctccagcagtttgggaacacatcattcctcttaaaaccctcctataataatccatttat

SEQ ID NO: 239 - DNA, 599 bp, Homo sapiens
gaaatccataatttaaaagagaagaaaataaatgtgcacttagtgtaactataaaatatttgtgttgaggttggattacatttagagtattttaaaagaaaagtatcttccactttaaatatatgctatttggtttaatgctgaaaaaaattcttaaaaattaattgatttatgacttacttacttcgagtataattagtgtaattttattaaatttaacactcgcttttaatccccctattccaacattagatatcaggtaaacccactctcatgagaatattagttaccttgttctascttcattattgtctttgtggagtgttataattatttttcttatcagttcattctgcacagttctggtatcacctccagcagtttgggaacacatcattcctcttaaaaccctcctataataatccatttattctctttcccttgagaaacttaccctccttgttgtttccaaagtcagccagcatccatgactctcctccttgccagacctaggctaccttcatcttgtggaaaatgtgtaacacatgaaggcatgggtttaaatgtgattctcgtatagaatagagagtagtaagata

SEQ ID NO: 240 - DNA, 599 bp, Homo sapiens
gtaactataaaatatttgtgttgaggttggattacatttagagtattttaaaagaaaagtatcttccactttaaatatatgctatttggtttaatgctgaaaaaaattcttaaaaattaattgatttatgacttacttacttcgagtataattagtgtaattttattaaatttaacactcgcttttaatccccctattccaacattagatatcaggtaaacccactctcatgagaatattagttaccttgttctaccttcattattgtctttgtggagtgttataattatttttcttatmagttcattctgcacagttctggtatcacctccagcagtttgggaacacatcattcctcttaaaaccctcctataataatccatttattctctttcccttgagaaacttaccctccttgttgtttccaaagtcagccagcatccatgactctcctccttgccagacctaggctaccttcatcttgtggaaaatgtgtaacacatgaaggcatgggtttaaatgtgattctcgtatagaatagagagtagtaagataggaatcttctgagagaatgagatgattggttttaagagttatct

SEQ ID NO: 241 - DNA, 599 bp, Homo sapiens
tcagcaaagaaaaatctcaccaaggccttaaataaataaagagtggatttgtcttactcagaaaaatgttcaagagtaggccatccaaggtgtgtatgaaaggttagtaatgttattaagggtcttggtttttatcaggatctacctatgattttggctctaccatatgtaatgtgtagtaccctatccacgaatattgctttcatcatgagttggatgctgcacctctaggtgttatatccatgttccaagaaataagatgataaaggggagaaagatcacaggatgcctacagtagartctgttgctttaaaaaggttttatagaagcatcaccaaatgactttcttatgaatcttaatgatcctaatatatacatgtgtatgtgtgtgtatatatatgtatatatatatatatatatatatatatatatatatatatatagtattttttcacatgattatgagatggctcatgtgattacagaagctgagacattccatgatcttccatctgcatattggagacccaggaaaaccagtggtgtagtccatagtctaaaagtaaggcctagagagccagtggtatagatgacaagtt

SEQ ID NO: 242 - DNA, 599 bp, Homo sapiens
gttgtaagagttaacttgcattaaattttgctagagcaagaggcatgttgcatatgttcctgattacctagagaaatgttttaaaaaatgaaaaaaatcccgacgccattatatctcttccccacatttaagtagcatatacttctgtgaaagtgtttctattaaaataaacagaaataagtaagtctaatggcttttcaaattttgtatgctatagaatcacctggaagtctcattaaaacacagtttgttgggcctcacagtttctgattcagaaggtccaaattaatcagggttttsggattgaaaatatgcctttctaaaaagttcccagatattgctaatgctgctattctgggccacaatttgagaaccaataacaggcctgtttccttcagactacacatacatgtctccttaggatgattctcattctcactcaaatacccaggttaacaaagactccactactagtatctggtagaaagaaagaaagatggggacttgcacaccagcttttacatgcttaggactgagagtagaatatgatatttctgctcatgtatcattagcaaaagcaagttgtgaagccatattta

SEQ ID NO: 243 - DNA, 599 bp, Homo sapiens
ataagctattaaatgaatccaactggattggttttgttgcattattgttgtgctgttttatcataaaatatttctgcgatttttgttagattcaatcagctttaaaaggattctacctatatatggttttcccatgttatggcttccctacttaacagctgtgtaaattattttcaaagctgtacaagaaaatcagcaagggcagaagtgcattttgcttcttgctttaagacatccagttatgagccagttatttaggaacttcaatttgtcaggtttgacaacattgtaattgattgyaataataaagcccttaccaaaagctcaattactttcaggaaatttgccttgttttcttaagtgttttgcaagtatgataagtaggttgtggcgcttttcttgacataagtgcataagagatatatgatctcccttagtgcacatagtgacaaaagatcaagctgttgggctgcaggctacaacaaattgaacccattgaatttcagctgtgatctaatgcagtgttaggtttgtttcagtccattcaagtgatacctttccatgcaagtgccataactgtatccacaaaactgatcaat

SEQ ID NO: 244 - DNA, 599 bp, Homo sapiens
catagacgcacatgcacacacacacagagtctcaattttcagcaagaatctaagcctcttgagggaatgaactgtgttttatatatatctttgtattcctcaaactcagcacaattgtgcccatttattttataaacatagtgaaaaatatttttgtgctaattactgcactgagattggagcttaaaaatttattttaacacctccccctgctcataaacagctcatagtctaaaattggaaatgaacataaaacccaataatacaaatcattgtaatcagtacaatgacaggaacatrcataaaatactatggaaaccaatatgacagttgcatctaacatcccggagacgagtaatgggagaagcaggagacctaagcaaatcttaaaggatgcacggaaattaccgttagtatgaatgtgatgagaagaaaaagaggagaaaatctgttttagaagacttcacattacaggtagagtcattgcatgagataaaacatgagggaagagtggtcgtgaagcaaggtatgccaactcctttagcatacatttttgcaaagagtatttggcttagtaataaaatgtgttaaacaaatct

SEQ ID NO: 245 - DNA, 599 bp, Homo sapiens
ttaaaaatttattttaacacctccccctgctcataaacagctcatagtctaaaattggaaatgaacataaaacccaataatacaaatcattgtaatcagtacaatgacaggaacatacataaaatactatggaaaccaatatgacagttgcatctaacatcccggagacgagtaatgggagaagcaggagacctaagcaaatcttaaaggatgcacggaaattaccgttagtatgaatgtgatgagaagaaaaagaggagaaaatctgttttagaagacttcacattacaggtagagtcrttgcatgagataaaacatgagggaagagtggtcgtgaagcaaggtatgccaactcctttagcatacatttttgcaaagagtatttggcttagtaataaaatgtgttaaacaaatcttaatcctttcatgcaatcaactgctctaaattaggatgtctcatcaactagatggatttcaggatccacctaaaactctctcctgtgaaaaataaccaattctagacattgcagctggttattcggggggggaaaaggcattttttgaaccagaatctttattatgataaataaatctatcaa

SEQ ID NO: 246 - DNA, 599 bp, Homo sapiens
atccatagccatttttgattcaattgcttatacatctttttccagagtgagttgttattttcttcactttcaactgagagataaacttcagagtatttgattgttactttcctgatatttcttcttggctggctttggctttggggttcacccagtgcaagttcatagaggtggtgagtcatcaaatgcctgagtcattgcaatgccaatgccatttttatagtgatgacagctccacttggctttttacagccctctcttaattccatttcattcagcctcagagccagactgccttakgattggcaacaagatgatcccatctcattgatgaccttaaaggccttccattctcaatggataaagaagtgcagataattttagcagtgcctttggtgcctctataggcacatccacccacatttaaatacacgtttttatgtttacccgtttatccctctctgtgaaatattgcagcagcttaaacattagcatactaatccaggcctgaggtctgggaaagatttatggtaaacatttttggtgccattattcacactatgtgtgaagctgagtaagactttcttctgaatcagagt

SEQ ID NO: 247 - DNA, 599 bp, Homo sapiens
aaatagtggagtgtcgatctggtttactcatttgctctgtggcagcacatatgaaagcaggtatgattttattgagtgtgtattattgttagggacttttcttgacaatggagatggaaaaatcaaaagagaatatatttacatgtatagatctcacaatctgaccagggtgtcaggggagcagaatatgttaagaaaaaaagctagtaagacattttagttttttctcagtgtctgatggggaatatgggaatacagaatagggagagtagagaaaggctggaaagaagacgtggtgayagatgatagaaaaagcagatggggatactagagggagtgggaaaaaccttagagctggagaaagattgaagtggaatgtccaggatgagaattgtgtgcaatcttaatctggtctgtgctccttatattatgtgaagatgaggcacatctgaaaccaagttcaccttatctgtggttccctgctccatgggagtaaaatacataaaggaggaactctttgattcagaagggcagctattttcttttatggcctcattgctcttgtaacttcttgaacttttcctctgtacacaggagat

SEQ ID NO: 248 - DNA, 599 bp, Homo sapiens
ctccacataatttttaaataaggcattttatacggcagctagcttcttctaccatgtatgttccaagattcgggaagagaagctgccaatcttttaaggcctggtccagaaactgttactggggtacttccaccatattctatccaactattcacagacacagaatccttgctgttgcaacctgttagctgtgttgccttacagtcaaattgaaatagaaggagacacagaatccatgtctaaataggaggagagacaaagactttgtggccatcattaacctatcacacttacctgccyggagtggtgcataagtaatactaaacttggctcaatatctcacattctccttagtgctcgatgcctcgatgcttcacgaggtcctgaccttctctatgataccccttttgcctttcttcttctggctaactttaactcacacttcaaattctagctcagacatcatcaactcctgccccaagcccctaaatagactccaatttaatatcatcctgatttcatgcctatctcacctcctgccatactttactgggacaatctgttgccttcaccaccttgtcagctctgtggtgggaaaa

SEQ ID NO: 249 - DNA, 599 bp, Homo sapiens
gagagacaaagactttgtggccatcattaacctatcacacttacctgcccggagtggtgcataagtaatactaaacttggctcaatatctcacattctccttagtgctcgatgcctcgatgcttcacgaggtcctgaccttctctatgataccccttttgcctttcttcttctggctaactttaactcacacttcaaattctagctcagacatcatcaactcctgccccaagcccctaaatagactccaatttaatatcatcctgatttcatgcctatctcacctcctgccatactttaytgggacaatctgttgccttcaccaccttgtcagctctgtggtgggaaaaagtatcatgttttattcatttttgtatttctagagtctagcacaatacttggtgtaccaatgtacccaggatgatctgaatgactgtcatcactttaaattttattgaaaatgtaccaagattattatatgaaataaattaattttagttcaaatgaccaaggcacttcaacagaaatatttagttttcaaatattgttaccagattaaatggtaaaatattaatttttataaagatgtgaacaatcttt

SEQ ID NO: 250 - DNA, 599 bp, Homo sapiens
agatgtgaacaatcttttaaaatattcaatcatgagactttatatatcttcatttaattacaatatccattttttttccttttgagggttgagttggcatagctagactgagaaataatttttacaagttagacatcacctagaagtcaataaaaatgatgaagaggctgtcttgatgctgacagagaaacagaattatatatgatctttcaagtatagaaaattaatctacatctagctatagatgtatttgatcacaacccaaaaattaaagtacaggtaagctccaaggaattttcmgctatttctaggtataacaaaacagcttgcagaactgctctacattcaaaattcttcagccaatctcctcttatacctttgatctttcttcttattttctcagttctctctcattggtgacagaaaatatcttcatgaatatgtatggctaaaatgtgttaacatttattgagcattactatatgctaagcactatactaaatgcttcacatgaaatatctctttattaagatgactctgaaaattacctgttatttatagtgaggaaattcaacttcagagggtgacttctccaaggt

SEQ ID NO: 251 - DNA, 599 bp, Homo sapiens
aacgaaaatttcaataaacccttggtcagaagatatctacaagatcataaaaagatgttaatcatactcccaataacccacctcaaaaccttgcagtaaattttgaaacctgggaactcaagtcttcgaagcttctcaaagttcttgcctctgttccttctaggcttaactaccaccatcgcttagctcagatgcagaacacctcacacctggaatattggaagaaccaaatcactttttctaaaatgtcactttattcatataactcctttgacctaaatgatctatatgaatattcartgctcctggaataaattctagacttcttagtctggtattcaaggttcttgatgatctcatgacaatctacttttccagcttcatctcctcctggcccttgatattaaccccaaatctcaggccatcttgtcttctctctgtccctgcacttgctctgatcttctttctttgctcataaggaacagaatattctctctcaattgtcatctctctaattcttatcaatgcagcagtgcccaatataaatggcatctcttccacaagcttttccgtatcatccaaaaagaagtgacacttat

SEQ ID NO: 252 - DNA, 599 bp, Homo sapiens
cagactataacatgacgttgtctgcagaccccaccgtgttctaagctggccactctcattagtgtgagtgcacatgttgataagccccagggggcatttatgtgacaccaaagaacgtttcatgacatgccttcagtcaagattttactcaggcattttgattctctctctagtgctatttcctctttttctgtagcttttatttgcacttgaccctatgttttagaaagtgcacacaagtcagtattccagacatcccagggacttgaagagctgatctaatgtctgtgttgcaagatraaaaacagaggaattttgttgaacccataaaaaggatgacattttgcctggccctgagctgttctgttcaaaattgaagcttttgcagcttgcggctccttgatttgtgagctagagagcatcatatcttggccattactgaatcataaaagttctctgcctcttcccagcccattttatttaaaaggacaaagagaaaccctttatcttataaaatgcattttctttttattttccttcattgccggaatgtattctgtaagggttcagtgaaaacatatgtaaatggtaatttgaaa

SEQ ID NO: 253 - DNA, 599 bp, Homo sapiens
tgtgccatataatcttgtgaaaggctagaaccctgtacatattaattattttcatcatctataaaatggtaatatagagccacataacacacccaggtcagggtctggtttaaccacaaaagactgattggttcaaggtggaatgaactgagcatgaaggagaaggcttcattaagaattcctctacatttaatataacttgggtaagagacagagacccgtgcaatgtttctaaacagcatctgaagtagtgatttcccagcttaactgtatcaccttataactaaacccatctctatmgcataattattcctttatgtaacaacatgtgttcagtgcatttgataatcagtcactgtcttggctgctggaaatcaatagcatagtcatccttcttcaaagagctttcagtctatttataaagattttcttatgtcaaacagataagcattatttaaatgcttatagtaggcatagtctcattgttatatagataaaagataatacaacatcattttatgtgaaaagaatatcagaaatttgcacaaaaaataagtgtgccacacagttaatttctaaaagcaccattttttgtgaat

SEQ ID NO: 254 - DNA, 599 bp, Homo sapiens
tcccagcttaactgtatcaccttataactaaacccatctctatagcataattattcctttatgtaacaacatgtgttcagtgcatttgataatcagtcactgtcttggctgctggaaatcaatagcatagtcatccttcttcaaagagctttcagtctatttataaagattttcttatgtcaaacagataagcattatttaaatgcttatagtaggcatagtctcattgttatatagataaaagataatacaacatcattttatgtgaaaagaatatcagaaatttgcacaaaaaataartgtgccacacagttaatttctaaaagcaccattttttgtgaattttgtgatgtttattgttttgtgagctttttaaaatccctaatccatcatggtttattttctcattctaaataaacattcacttctgtacataattttgtattcttgttcttaaagtattcattgaaactctagaaacttgtgaccccacacagcctcagtccagctctggtacaacactgttatctgagatgatctacacaaagtcacaacatagtaccttgcatgaaataggaggttaaagcatggcagggatg

SEQ ID NO: 255 - DNA, 599 bp, Homo sapiens
atcctgagtaacacggtgaaacgacgtctctactaaaaacacaaaaaattagccaggcgtggtggcggacacctgtagtcccagctactcgggaggctgaggcagaagaatggagtgaacccaggaggcggagcttgcagtgagccgagagtgcgccactgcactccagcctgggtgacagaccgagactccatttcaaaaaaaaaaaaaaagtatgaatcctgtaagaaaacaaatctagaaactctgaagccccttgtaaattgaaagcacagttataggcagatagaggtggaaacmaatattctatataaatgtttactcttcctccattttgaaatgtctgttggaatagtggagaaaaaatgagtgtgaatcgaacagttctttggtgaagaaaactcacaggtggttaatgttctctatagcgagatgtcctcaatagtaatttctctttttctcttctgaaaaagatggagaaaatattcagggtttctcagtgcttggaaagaaaaatacagccatccttttcgttggcttcaaatagtctaaatagtgtcaacaaacccactgcagctgagtcagaaggaagatagcca

SEQ ID NO: 256 - DNA, 599 bp, Homo sapiens
ttagactgattgaaaaaattaaagcacattttatttacaaaggaaatatatgaaataaaaacattacaaatgtaaaattttaaaaattttaaaagaataccaagataatttctcatttaaaagaaaaggaggaaaagcataggttatagttgtaaaaacagaaaaaaacaaacctcaagttaaaaattaagtgaacttaatggagccattttcaatttctcaaaatacattcttcataacgataatatcattatgattatctatattaaaataggcatctctctacatatacacacaacwaacataagatagtttttaaaagataaaatactttcaaaaactattttaagaatttaaaatagttttaagggcaatacaaatggaaaggaaagcaatagttataacctctcatacctcttattatgatactaatttataatgatttctaatttactaaaaaaatggaactgaacttttaagtacctaagaaacattaatgagaattcatcaaatcagcccatagggaaaaaaatcaatatgttccccaaagaacaacctatgaaggctatattctctgaccaaaatttattgatatgtga

SEQ ID NO: 257 - DNA, 599 bp, Homo sapiens
aaaaaaatgtggcactaaataaactccaaaaaggtcaatggtttttagtatcacagctgaaacaaaaaggcagagcatcattttgtttaacgttaagctggtaatgtatgcagtaagagactgaattttttttgctgctctgagcatgtccatgaatgaccatgaaagaaccatgaatatggatcttaaagttacaaataaattttagcttgtaagcagattcatgaatatggaatccatgcataacaaggatagactgtatttgcatgttatctgaagaaatttatatgtatttgaaaytgtatgctccatatgtttccaactttataatataagatatttcttggaaggatatactgatgatttaagaaaaatagtcaatattaataatatgatttcaggaaaagataatctgagctttctagttataaattttttcaaaaacgccttctgttaactagagagtaccccaatgtaagtattaatggccaatactacctattaaggactgaaagtttatgttccctacaaaattaatatgttgaaatgtaattcccaatgtgatggtatttggaggtggagccttttggaggtaatta

SEQ ID NO: 258 - DNA, 599 bp, Homo sapiens
tggatcttaaagttacaaataaattttagcttgtaagcagattcatgaatatggaatccatgcataacaaggatagactgtatttgcatgttatctgaagaaatttatatgtatttgaaactgtatgctccatatgtttccaactttataatataagatatttcttggaaggatatactgatgatttaagaaaaatagtcaatattaataatatgatttcaggaaaagataatctgagctttctagttataaattttttcaaaaacgccttctgttaactagagagtaccccaatgtaaktattaatggccaatactacctattaaggactgaaagtttatgttccctacaaaattaatatgttgaaatgtaattcccaatgtgatggtatttggaggtggagccttttggaggtaattaggtaggtcatgagggtagagccctcttgaaccagattaatatttttataagagacagagagcttttttcctctttctgtcatatgagaagataagaagttgacagtctccaactgggaaaaggacctgtaccagaatcagaccatgctggcaccctgatctcagatttccagcctccag

SEQ ID NO: 259 - DNA, 599 bp, Homo sapiens
tgaatatggaatccatgcataacaaggatagactgtatttgcatgttatctgaagaaatttatatgtatttgaaactgtatgctccatatgtttccaactttataatataagatatttcttggaaggatatactgatgatttaagaaaaatagtcaatattaataatatgatttcaggaaaagataatctgagctttctagttataaattttttcaaaaacgccttctgttaactagagagtaccccaatgtaagtattaatggccaatactacctattaaggactgaaagtttatgttycctacaaaattaatatgttgaaatgtaattcccaatgtgatggtatttggaggtggagccttttggaggtaattaggtaggtcatgagggtagagccctcttgaaccagattaatatttttataagagacagagagcttttttcctctttctgtcatatgagaagataagaagttgacagtctccaactgggaaaaggacctgtaccagaatcagaccatgctggcaccctgatctcagatttccagcctccaggactgtgagaagtaaatttctgttctttctaagcttccctgtcca

SEQ ID NO: 260 - DNA, 599 bp, Homo sapiens
attaaggactgaaagtttatgttccctacaaaattaatatgttgaaatgtaattcccaatgtgatggtatttggaggtggagccttttggaggtaattaggtaggtcatgagggtagagccctcttgaaccagattaatatttttataagagacagagagcttttttcctctttctgtcatatgagaagataagaagttgacagtctccaactgggaaaaggacctgtaccagaatcagaccatgctggcaccctgatctcagatttccagcctccaggactgtgagaagtaaatttctsttctttctaagcttccctgtccacagtgccttgttccagcagcctgaagtaaggcactacctaaagtatttagaggccacctgcctctgagcacaatcaaaaaataaagttaatgcttccctaaaattacaaagcaaatcagggtatcaatataatctaattcatagaagttaattcaggctcaagtttatgtagatatgacctgtttgacaagagtaaaatttaattataaaaatttcccaggattatctgtcaaataatccatcagagtttggcatatggagtttttggaaaaaaat

SEQ ID NO: 261 - DNA, 599 bp, Homo sapiens
agacagagagcttttttcctctttctgtcatatgagaagataagaagttgacagtctccaactgggaaaaggacctgtaccagaatcagaccatgctggcaccctgatctcagatttccagcctccaggactgtgagaagtaaatttctgttctttctaagcttccctgtccacagtgccttgttccagcagcctgaagtaaggcactacctaaagtatttagaggccacctgcctctgagcacaatcaaaaaataaagttaatgcttccctaaaattacaaagcaaatcagggtatcartataatctaattcatagaagttaattcaggctcaagtttatgtagatatgacctgtttgacaagagtaaaatttaattataaaaatttcccaggattatctgtcaaataatccatcagagtttggcatatggagtttttggaaaaaaatagatggctaaatattagaataagccccttttccaaaccataataaagttctgtcttcgggatggcagtaccataaagaggagccagtttcattgtcaatgtgtctctccttctaagaaggctttacttttagagaataaacacaaggcaa

SEQ ID NO: 262 - DNA, 599 bp, Homo sapiens
atatacatcctaattaaggcaggaaatgtaattctgtccttacaactattgggctcagaaactgatttgatagcttaaactataacaacaagggtgtctgggagaatgaaccaagtcactgccttcacctcagccctgatgaatatgcctcatttttatttctggtttacttcctcagtttctgcatataactacaggttcattccaggactaatcctttgctctggatctcagatgtctgacatctacattgctcttgccagtcacatcttcctgaggggtactctttttaggtctagscactctgtggaatgctgccagtcagctgacaccaactatcactgctacttgaaggtaccaaatgcccattctattgacccatttgaaattacaggggaaaacaaagctggcctgacacaagcctttgaacaataataagctcccctcctcagggcctaggccaacaggtaattcagctctcttttcacatgtccacttctataattctaccttattctcttttcccactattccagtgttataatccaactcaacttcaattggcgtacatattttaagcaagaggttgtggtaagatg

SEQ ID NO: 263 - DNA, 599 bp, Homo sapiens
catttcatcaacagaatgtagaatttatatggatggaaaaatgctataaatcacaagaagaagcactggaacgtttagacattctgaaattattaatttttcaaagaagcctctgtccacctttcctttggagtcttctctgctaaagctccaatccattaattagtagctacattgctctgagtaaattatttgacttctctagcctttttgtctttataccttaaaatgcaatattaaaattcataaagtgactcctcctcttgttagcaggagcagcacagtcctttgcaaagggtmatgaacacagggaggcttattcattaagagccattattatagcagtatgccatatagccccaaagaatccagaatgcagtgctaggttgggaaaaagttccaccagaaagcagaagaaggtaatggaaagggtggggaggcaatacatatatatgttctaatctcctctatatgaatagaattcccagagcagggagataagtcaaataggttgaattgaaagtcaagagtgagaatagagcttgaggccgatatactaccagaatttctggaaagaaacttcctcatggagcatccac

SEQ ID NO: 264 - DNA, 599 bp, Homo sapiens
aaatcacaagaagaagcactggaacgtttagacattctgaaattattaatttttcaaagaagcctctgtccacctttcctttggagtcttctctgctaaagctccaatccattaattagtagctacattgctctgagtaaattatttgacttctctagcctttttgtctttataccttaaaatgcaatattaaaattcataaagtgactcctcctcttgttagcaggagcagcacagtcctttgcaaagggtcatgaacacagggaggcttattcattaagagccattattatagcagtrtgccatatagccccaaagaatccagaatgcagtgctaggttgggaaaaagttccaccagaaagcagaagaaggtaatggaaagggtggggaggcaatacatatatatgttctaatctcctctatatgaatagaattcccagagcagggagataagtcaaataggttgaattgaaagtcaagagtgagaatagagcttgaggccgatatactaccagaatttctggaaagaaacttcctcatggagcatccacacagagatatctcacaatagggcagctgcaacaaagttaatccagca

SEQ ID NO: 265 - DNA, 599 bp, Homo sapiens
gagagtttaccaattttgttatacgtccattcatatcaccctcatagttcaacatgaaaaacatatttaaaaacaacttttagtaatccatttaatagccaccaggctcttttatcaaggttcctacatataacttcttcctgtaccagcacggtgccattgcatgatgtggtccataggcaggactataagcatgaggcatggcacatgggcagtcacattgcaaatatgtaacagtcaggtcggagagatgagctccttacaaagtacaaggaagtctaaacaaaggggtcttacagmctacctttctggctccgtcatccaggctggagtatagtggcaccatctcggctcactgcaacctccgcctcctgagttcaagcgattctcctgcctcagcatcctgagtagccgggactacaggcatgcgccaccatgcctggctaatttttgtatttttaatagaaatggggtttcaccatgttggccaggctggtctcgaactcctgacctcaagtgatctgcccaccttggactggcaaagtgctaggattacaagtgtgagccaccccgcctgacctgacagcctacctttctag

SEQ ID NO: 266 - DNA, 599 bp, Homo sapiens
atctcttttattacatattgttctgaaatactattattctataaattatagtatactctagcaaaatgtattatgctatatatactatattatactattgtacactataccacaaattagttatgttataatataagactatattatgctatcgtatactgtactaaaaatcaaagtcagttttttggccgaatgacttaaggtaattcattccagtcattcaggaaggcattaaactatgtaaataagaactgtccaatttttaatagttagtgtaacccaatcagatcatgtactcarttgtatcacattcatggctttaattttaaacattttgacagaattttttaaaacccaggtttaatggtcatcttcaatgataagaaaacggagagatttcagttacaatagtgactcagtcactatgtttgtatgaaaattagtcacagagatgctcaaataaccttggtaaatagtagattcaatactgattcatagacatgaattacaattttgactttattattaagaatgtggttgatttaaaaagatcaatgtaaattgccttaaaaataatttctttgaatctaagaatccat

SEQ ID NO: 267 - DNA, 599 bp, Homo sapiens
catcacatagagtttcctctaatcacatggtcccttgacttacaggatgatgcttgataggctagaacccaccaaagaatggtagatgaggaagaacgcagagataacatcaacaacaaaatcagacccagcctcccttcccatccctgcctacctatctcacaccaggcctgctctgggggaggggggaagggagagatgtgaattggatctgagatggaagtgtgattaagttggtcctggaactagattgagaaatagaagtagtcagagaagctgtggggtgtgcctgaaatgccrccaattggaatagaagaaggatcactgctcctggaaaaaaagaatcaacaaggaggttatctgatgctgccccatcaagttcagttcgtataatgtcaattatacctaagtataaccaaagttccccagtgctcatgaagatgctgtataaaacaattggcctttcatctctcataggtatctacaaatacacaacgcaattttgagcattttccttcaccatatttaagtaatgtatacatgaaaacacacatacacacaaccttttgacaaggaatacaaaattgtgtagtttccaa

SEQ ID NO: 268 - DNA, 599 bp, Homo sapiens
accaattggaatagaagaaggatcactgctcctggaaaaaaagaatcaacaaggaggttatctgatgctgccccatcaagttcagttcgtataatgtcaattatacctaagtataaccaaagttccccagtgctcatgaagatgctgtataaaacaattggcctttcatctctcataggtatctacaaatacacaacgcaattttgagcattttccttcaccatatttaagtaatgtatacatgaaaacacacatacacacaaccttttgacaaggaatacaaaattgtgtagtttccarattcttccaattatgtacattaattatgtgcagggttttttttttttttgtatacgaattatacctcaacaaagctggaaaaattgtggagaaatcagcattcacacttcctgttattgtggttcacaatcacctctaattttcctgagaggatagacagttggattgatctgaaaatttttcctgagaggatagacagttggattgatctgaaagagaaaaggaaaaaaagaaattgaggagcctagtacagaccttaggctttctttatttaaaaatataatatgatgtaagtatca

SEQ ID NO: 269 - DNA, 599 bp, Homo sapiens
cagtgctcatgaagatgctgtataaaacaattggcctttcatctctcataggtatctacaaatacacaacgcaattttgagcattttccttcaccatatttaagtaatgtatacatgaaaacacacatacacacaaccttttgacaaggaatacaaaattgtgtagtttccaaattcttccaattatgtacattaattatgtgcagggttttttttttttttgtatacgaattatacctcaacaaagctggaaaaattgtggagaaatcagcattcacacttcctgttattgtggttcayaatcacctctaattttcctgagaggatagacagttggattgatctgaaaatttttcctgagaggatagacagttggattgatctgaaagagaaaaggaaaaaaagaaattgaggagcctagtacagaccttaggctttctttatttaaaaatataatatgatgtaagtatcaggttttgatagagaaaacaaattatagaattaacactattggctcttgagagttcttaatgtgttcgagtactatattttacatttacacaaaagaaagacaacactcattaccttaataattgtat

SEQ ID NO: 270 - DNA, 599 bp, Homo sapiens
tgacaacttttctctaatccacaacttaaatatttgctcagaaatttgttcttttgtgagcaaaaatgttttactctatggtaaattatctagtctccatatttgtcctcttcattagagtggaggttctttatgagcagaaatgttctggttcactttttttcccattttcaggttaccctatccaagctgtctttgagaaatggcattcaccactactatggcttagactcttgctcaggttaaatgcctccaagaatgacaaaatgagatttaatatttctttcattttcctttctrttgaacgtgtgtgtgtgtgtgtgtgtgtgtgtgtgtgtatatatttttttttttttttgaaacggagtcttgctccgttgcccaggctggagtgtagtggcatgatctgggctcactgcaacctccgtttcccgggttcaagcgattctcctgcctcagactcccaagtagctggggttacaggcgcgggctaatttatttgtatttttagtacaaacggggtttcaccatgttggcctggctagtgtcaaactcctgacctcaaatgatccgcccacctcagcctcccaaagtgctgg

SEQ ID NO: 271 - DNA, 599 bp, Homo sapiens
tgtagtggcatgatctgggctcactgcaacctccgtttcccgggttcaagcgattctcctgcctcagactcccaagtagctggggttacaggcgcgggctaatttatttgtatttttagtacaaacggggtttcaccatgttggcctggctagtgtcaaactcctgacctcaaatgatccgcccacctcagcctcccaaagtgctgggattacaggcatgagtgaacctgtctatcttaatgtcctatggcaaccaccagctcaaaccctttaaaagtaaactcataatctttcttggtstttgcattgtttctcttcttttttatattttcttagagaatacagacatttcagagattctgcaccaaatttatcttccaatttggttccacttctccaaattcgttcatctcttgcctgaacttctgaaatagtgtcccagctcttctacaaccagaatttcttttatctagcaattgactaattatgtttatttattcattccacgaagattaattgggcaactacaggattctaggaattctttcatgtgctggaaaatcaataatgaatacctccaagctactgaagcatttctt

SEQ ID NO: 272 - DNA, 599 bp, Homo sapiens
atgatccgcccacctcagcctcccaaagtgctgggattacaggcatgagtgaacctgtctatcttaatgtcctatggcaaccaccagctcaaaccctttaaaagtaaactcataatctttcttggtgtttgcattgtttctcttcttttttatattttcttagagaatacagacatttcagagattctgcaccaaatttatcttccaatttggttccacttctccaaattcgttcatctcttgcctgaacttctgaaatagtgtcccagctcttctacaaccagaatttcttttatctarcaattgactaattatgtttatttattcattccacgaagattaattgggcaactacaggattctaggaattctttcatgtgctggaaaatcaataatgaatacctccaagctactgaagcatttctttatatgatcacagtgtaatctttgcaaagctaatcatgtcaactctgttaagaacctgaatcaactctgtattgcaaacaggctatggctgtgcatgttatagaaacctatttatgttaaggatccagtctattttttaaaaaacctcttctccaaattactcaatttctagc

SEQ ID NO: 273 - DNA, 599 bp, Homo sapiens
tcaactctgtattgcaaacaggctatggctgtgcatgttatagaaacctatttatgttaaggatccagtctattttttaaaaaacctcttctccaaattactcaatttctagctaacaaaacttctgcccaatctccaagcaatccttgtttttttctggccttttgtacatggtcatttatacttctgctctttccttcaactctgtctagggaatggcccaaattgtaataaccagtttaccatcttctccataaaagtaaaatcggtcagttaaccattttctctgggtcttcttcwgcttcagtgccatcaccaatcatcaaaccctctagattttattttctgactgcacagctcaatgctttcttactgctagtagaaaacaattctctatatactcccaaaaacttacttttttttaatcttccccataccttttcctaccctgctaatattcaaatactacatgcttataacagaatcaaaacatataaagacctaatttgaatcttacatttgggaagatttctagaactatagactattttaccttccttctttttcaacatagaatttctgaaagaaagcaatcccat

SEQ ID NO: 274 - DNA, 599 bp, Homo sapiens
cacaatcaacttagtttagatttatcatccataaggtggtcactcagaggtgaggaagatgcctgcagtttatttctagcatgctgtgaagtattttcccagtcaaggaaaataacaggcattagtagcattgtattcatttctcttctcataccactgggcaccaaaaagaaacattcttttagtctagggctattacaacacacttacattcaacactggtttgaatttaagcgtgtgtttatagaacgctctcgactaaatagaaaaaaaaagacaggtgaatttccttgtgattgraaagcagttgctaagcctttaggaaagctgtcaagagattacacatgattttcaagagtagagataagagattatcttaatccctaaaaaagagcagagtcaagtgactgtcaaagaaatgaagtaaaatctgaaccatcggttcctccaacgcagtatcattttgaattgctgttttaggtattcattgaaaacaatatactccttggtaaactatctgaattttgcattagggtatgaagaatttggggatttaattcttgtactctcatggaaatttctagtaacataatttccta

SEQ ID NO: 275 - DNA, 599 bp, Homo sapiens
tcattagaatgtatgttcaaaaaccctttaagggtcaaaaccatttcaatccaaacgagggtaacaggaattattgtgtgtttatggtaacaatgaacaggtgaatgatttcaggtccagcaccctccatttgcaattatgacattttctttcctatgtgaacaccatcagaaatctttgatttctggcctcagacttctgagaattcttagtcactatccagctttagtcattcaagaaattttcagagtgtttttcattttgaatacattggcacacactcgaagactacaaaacaawgtcttcatgctaacaccaaaggataattcctatgagaccagcgagtagaaggcaagtcttgaatttgagacattttctctagatcatttgatctttaacatttcaacttgaaaatttgctttcaatcttatttaaatgtcatgaatttgggtggaatcttgaaaatatttaagtggttgataatgcattatttcaatgcaggtaacactttccaaataaagcattaactacaccatatggcaacagaaatcctgtggaaaccacaaaatgtgtggtttagaaattcattgatttcactg

SEQ ID NO: 276 - DNA, 599 bp, Homo sapiens
gttactactttggaaagatatttcccagactattattttctgaaagataattcctaaagctgtcattcttaccttggttttcctttctctctctctctctctctctctctctctgtttctatctctctcattgctaatggtacttggaagttagttaaaattctcagggaatagaaacaattattaaataactttagatacctgatatgcctgacatgaccaaatatgccctaaatttatactttctgggctcagatcgaagttcagatctaagttcaaagctctaaactagaagctttmgtatttttatagattccaagggcaaggctatgaattttttacttttagatgtatttttttactgtttgtaaaagtcggatatcataaatctggtagtttcaaaaaagctgcagatttttcaatagtggcccttgtatttcgatttaaaccaaatgggaccaaacttattacttctactctgaaaggtcatttttcatcaagattgtattagaaaataaatcatttaaagccatcctttggatggttccttgcatgaatgtcttaagaagaacacgtaataagagcctctttgcaaagcc

SEQ ID NO: 277 - DNA, 599 bp, Homo sapiens
ttaaaaaaaaaagtttctctcaaatgagttctaaattcatggttgctgataatgccaaatagttattccaagaagacacagaaaaattggtttagtcttatttttagattaagggggcacaaaataactccatctgtatcgtcacaggggcttcatttatttaatgtatctttttttgtcatcggaagtgggggcctcttacagggacctctgaaatgttttagtccctttatatatatatacatttaaattaggatttttgtatgttcaaacttagtctagggttttggaattatctgyctctttcccataaatatttactaaactcaaccacacaaatgtaaagttatacaggcttttagtgctttcctaacgtggttaatactcatattgaaaataccataaattctgattaatggcagataccaacctatggaccacatagtacaaactcatgaagagtaattgttttctcatgttatttcatctaattttagtgtgtgttaagggtgccttcggagaggacttcacacccctacccctcgccattgtgtatagacatttgaaaattccttaactacattatctctgtgtgtgtg

SEQ ID NO: 278 - DNA, 599 bp, Homo sapiens
taattagttcagaaactaaaaatatatatgtaaataatcacatgataaatttacataagataggttagtaaaggtcatttaatttcttacattcttgcttttattctctagaataccatttagtatgctatacattttctcagttcatgtgttaaacatgctacttcagtttgcccatttaggagcagactatattttatgtttaaatgagtagaaacattttaaacacattttcatctaatttggttatctggaagatgtatctaagataaggaatccctttgagttttagaaatttgyaaacatttattgaccaagtacttcattagaaaaacaagaaaagtttctgagcaagcacttctagtgcatgtaaatgtagttgctcatacatgatgtatacttagcattctgtaaatatatattcaaaaattatacacagaaatgagaaatgtataatagttgagaaaatattatataatattttagttattttaattgttaagtgactatttcaaaaatactgattgagtaaagtttttttcatttcagttttttaagtttttgtaatagaataattcaaatgtctgagtttaagaatt

SEQ ID NO: 279 - DNA, 599 bp, Homo sapiens
ttcttacttaattaagatagtcatttattatttttagaaataacctatttaaaaatttgctgtaatatggctagtaaatggacatctttcttgatatcaatcagttgttctgaaaaattacttataatagtttttattttatattttatacaaatatttgcaaagtcaagaaaaacttttcatttgtttaaacagataataaattctatgtttaagtgttttaattgtgcacatatgaaagattagttataacttcacgacattgaacttaaaatcacctaagatccaaacatttccaartatctctcctcaaaactttctttctgtggcataagtctttcccaggaaatcattatcttttcattcattgttaacattttacatgtaagtctgtttttaatttctgctaagataccattttaccaattgtcatcaaagacaaaatcagaagttgtatcacttatttttctaagctgtgtatggtacaaaagatttttatgaaagccttttttgtattacaaagtgtttcaaaagtctactatttagcaaagtatagtgcacttactagaaaacacttcaactgcaacatattgcagtat

SEQ ID NO: 280 - DNA, 599 bp, Homo sapiens
agttgttctgaaaaattacttataatagtttttattttatattttatacaaatatttgcaaagtcaagaaaaacttttcatttgtttaaacagataataaattctatgtttaagtgttttaattgtgcacatatgaaagattagttataacttcacgacattgaacttaaaatcacctaagatccaaacatttccaagtatctctcctcaaaactttctttctgtggcataagtctttcccaggaaatcattatcttttcattcattgttaacattttacatgtaagtctgtttttaatwtctgctaagataccattttaccaattgtcatcaaagacaaaatcagaagttgtatcacttatttttctaagctgtgtatggtacaaaagatttttatgaaagccttttttgtattacaaagtgtttcaaaagtctactatttagcaaagtatagtgcacttactagaaaacacttcaactgcaacatattgcagtatgatgcaaggaaaggaaagcatgagagtcagttcttcttgcccactcttcctccaggattctggatgtactcaactatgttatgtgaccatatgacacactgg

SEQ ID NO: 281 - DNA, 599 bp, Homo sapiens
aggagaatcattcttcatgaagcatcctatagttatcttccaggaggaataacaaaattgacatacagaatgtgcatagaagcacttaatgggaaataattttatttctactgaggataaactagattgttaacatattcaattcacccaacttccccacacctccatttcttccttcagaatttgaagtagtcaaattagatgaccactcagatttatccagctctgaaatcctatgatttctagataattaaaacagccatgtctgcaattaataatactataccaataatacaggamtgaagagccctgagttcttccacttagatgctattaacaaaaatcaaaactctaagtgaaaatctatttgcatgtaaaataggggcaataatgtaaaatttctcatggaattatagggagaattagatgaaaaatgaatgcacagtgattccaaaagatagctctgtataagagaagtaataaataccattcactctctgtgaatctgtaagattgcaataaacattccctaccctatttctgtagctccttgctgataacagtttttatctcgctgaatgctgctgctagtccctcag

SEQ ID NO: 282 - DNA, 599 bp, Homo sapiens
taagtaacgactatttacagggaaatgtaagctctcagggaaaagctataagatttcttacatcagcagagcacaaatattacaaaagaaaataatcataccaaaaatcatacaagggataaagccaaatttgagttccaaataaaacaagaaattaaagatactgaatattttcaaagagtaacagagaatggtaacatgacttatgaataaatgtttataagaagacaattggaaatatatggtataaaaacatgttaatgtgcataaagcatcgaaaagaagtgttgaataacaaamtgagcacaatggaagaatgtatcaatgagctggaagatcaaatagtgaaattttccaggaagtattaggaaggcataaagagaatataacgtcaacttaggacatagccatttagacaggtaaaatagaagtgtcaaaatctgtatagaggcagtcaccaaaataaaaataaagtataaagaagaaatatctgaaaaagtattaactcagaaatttccagaattaagaaaaataaaaaagtaaaatgccagggaggagggagaaatcctaaatacttgtaagttgccaaatagaatatt

SEQ ID NO: 283 - DNA, 599 bp, Homo sapiens
tttgtctaattaatgtagcatttaaatgccagaataaaatagacattctcagttataaaaggcttcagaatgttttcaccaaaagaacatgtatgaaaacaaccttaaaatatttcggcaaaataaaggaattaatcccaaaagatatactaacaacaaggataaataaataacttagaagaaaaatgtacctcattctgcatctggatcatagccagcttatagtagcccaacttaccctcttagggttcattgatctctaggcttaactgcactgcagttactgctttgtcaactagsctgatattactatgtaatgggagtcaatttagatcaataatacttggaaaatttgagttacatagaaatgaatgaagggtgtttactacatgggtgatactcaacaaatgtatttaacacttgcaaatttcaccttataaattttgatctgaaaaaattaatgaagtatcatctctctagctcaataatagacacacagtagccatcagaaaacatcaagttcattttttcccttcaaattaccagtaatatgggcctaagcaagtgagaagcccacactggagagctgctgagtcaga

SEQ ID NO: 284 - DNA, 599 bp, Homo sapiens
tggaaaatttgagttacatagaaatgaatgaagggtgtttactacatgggtgatactcaacaaatgtatttaacacttgcaaatttcaccttataaattttgatctgaaaaaattaatgaagtatcatctctctagctcaataatagacacacagtagccatcagaaaacatcaagttcattttttcccttcaaattaccagtaatatgggcctaagcaagtgagaagcccacactggagagctgctgagtcagatgagcatgtgcacattctaagagtactgtattgtgttgatttttrtcataaattatggttaaatgccccttctagctataagctgtactagaggctctgctctaatactactctagggtttctgcctcctttctggtatctataaatttttgaaaatcccaaattttccagtttatttagctcaacttttagaggacatacacagaaacttattattgatataaaaatgatgtcttctggataaaatacagacttttttttcacagaaccaaaacaatacatatcaatgatacataaacataaatttacatatgccctactcatgtcaacattacaaaaagtgg

SEQ ID NO: 285 - DNA, 599 bp, Homo sapiens
cacttgcaaatttcaccttataaattttgatctgaaaaaattaatgaagtatcatctctctagctcaataatagacacacagtagccatcagaaaacatcaagttcattttttcccttcaaattaccagtaatatgggcctaagcaagtgagaagcccacactggagagctgctgagtcagatgagcatgtgcacattctaagagtactgtattgtgttgatttttatcataaattatggttaaatgccccttctagctataagctgtactagaggctctgctctaatactactctaggktttctgcctcctttctggtatctataaatttttgaaaatcccaaattttccagtttatttagctcaacttttagaggacatacacagaaacttattattgatataaaaatgatgtcttctggataaaatacagacttttttttcacagaaccaaaacaatacatatcaatgatacataaacataaatttacatatgccctactcatgtcaacattacaaaaagtggcaatcatgaacaaccatgtgaataatttagaacataaaatttcatttcaactattgctattgcttgaatgtgt

SEQ ID NO: 286 - DNA, 599 bp, Homo sapiens
ggcatacatctagtttctatttttgttagtcaatatttcttaccttcaatttaaaaaaaaacttcctattttgctgaaaagaattagaacacttggctttggatttaatcattatccctagcaatgttttagagatagccaagtgtttcacattttacaagccatacattttatgtttaatttaattttaacattgatctggattttaaaatcagcattattagtcagaataatgggctttagagggtaataagaaagaatcattaaaaaattgtgaaggaaatgctaagccattattcyggatcactggatgtttctttgccactcccattgagtttgaaattttatagcctggaaaagagtagtgaatatgaattactgctagagacaaagcctaaagtaagttactgcctacccatcaaaaatgatatgacatcttaaatttatagtcagaggaagttttctttaatcacttataatgctggttttaaaagatgatatgtatatttttaatcatctattttaccactagctgctaaagagaccttttatgtttgtgaagtgtgcaaccctctctagaattttctataggagatcta

SEQ ID NO: 287 - DNA, 599 bp, Homo sapiens
aagccattattccggatcactggatgtttctttgccactcccattgagtttgaaattttatagcctggaaaagagtagtgaatatgaattactgctagagacaaagcctaaagtaagttactgcctacccatcaaaaatgatatgacatcttaaatttatagtcagaggaagttttctttaatcacttataatgctggttttaaaagatgatatgtatatttttaatcatctattttaccactagctgctaaagagaccttttatgtttgtgaagtgtgcaaccctctctagaattttcyataggagatctattcttagaactttaaattgtgtgtgttttattataaataaataaataataaagctttgtatttagaaataatttcaaacttacaaagaatttccaaaaatagtacaaagaccatccataatcctttacctcaattcacccaatgttagcattttgccctatttgctttataatttgatctctttctgttatataaaggagatgataagtcaaataagcaacaagcatctgaacatttgcaaaagtattgttttctatcaactttatcataaatgatataagtcaatg

SEQ ID NO: 288 - DNA, 599 bp, Homo sapiens
tttatgttgtattccagatgggaaagcataacttgaatctaattataagaaaacatcagacagacaaaaagaggaacattctatttagaaaggaggtatggtaaagattagtcagcaaaaaaattcaatgtcatgtaagacatagaaaggctaagaaactattctgattaaaggaagctataaagatatgataactgatccaaggcctgattctatacacgagaaaaacttgtataaaggagattactgagtgaagtaagaagcagaaaaaggaacagttcctagagtgatattggagcraagcatctgacctgtcacaaaaccagaggatgcattgacttatatcatttatgataaagttgatagaaaacaatacttttgcaaatgttcagatgcttgttgcttatttgacttatcatctcctttatataacagaaagagatcaaattataaagcaaatagggcaaaatgctaacattgggtgaattgaggtaaaggattatggatggtctttgtactatttttggaaattctttgtaagtttgaaattatttctaaatacaaagctttattatttatttatttataataaaacacac

SEQ ID NO: 289 - DNA, 599 bp, Homo sapiens
aaaaggggatattccaccacttctgccatatttaacagtcaatacttggcatgttactataaacaagatattcaattcttccttctttcaatttctttttaatctatttattattgatatggagtcatagatccctattttttccaatcctttgtcatccattactctccttattttggcactcaaattgtccttgatttggaaagtagaagcccctttaaggtgactccaacgtctgtgtgacagaacccatcattgtgtttagcactttcatttttagcactttcttgctttctggcstaacgcaatattctaggctcatcgtccctgccccaaccctgtactcagtaatatttcaaggagccctgatttcttttattggaatgaggtagtagaaaccaagatctgggtcctacatgtgtttatcaatacctgggtgccttgtttgtaagccttttcagtggacagttaggaaacatggcctgtttatacagccatgcgtatgtcaacacacataaatgcataaacacacacaaatatatacacatataactgaacatacatgtatacgtgaatattttagaaatcaacagtccatt

SEQ ID NO: 290 - DNA, 599 bp, Homo sapiens
caatctggattctgctcatagtaatcaatactttattttcagttctaacacaatctccctgatccccatccttctgtgcagcatatttcctttatagtaatttgtcttcataactatttcgtctgtttgcatctgcatattctattataaactactcaaaggcaagtggtgtgtattatttatatgtttaaatccaacacctagaaaatattaagcttagtgagtgatttttgaataaacttaaaattgaatacatttgaaatggattgttatgctatgaagtcaggcctaagtatataygtaatcagtagaatagattgattttttaaataaaggaaagttttaattctattgctactactatcacgactattaccaccaagaattttgagtatacagcttctcttttttttctgactaatcagttattaaattctttccgcatcagatattgtgttaaagtctctgtgtcatttactcttcacaacatctctaaaagtgggaattattttcatgcctatttcacaaatgagtacagtgaagctcagagaactaagagactatcaaggcacatggttctgaagtaggagaaaagggaa

SEQ ID NO: 291 - DNA, 599 bp, Homo sapiens
tccacataatgttataggcaggaaaatattctggtaaagaggtaaaaaaaatccttgtgcatataacttttactcagtgggtttcgttaatttctaggcaattgtttacatactgagagatatgaataaataaataaacaaaacagttatatgatatgatgacagaatattgtaagtcctccagagaaaaaataattcccaataggaatggaaaagagtgaatatctagaaaaggtgtgtagccacattctttctgaaggatgacaggtgagagacctgagaatatccaggaaaagggcrtttcagatatgccaagggcttaacaagaaggtccatctggagtcactgaagaaaagaatgtaggccttgctaggccaatattcaaatttggattttcttctaggtgaaattagaagctattggagaattttagagagccaatgtaatcagttaagtttttaatacataattcaggccattatttggagaatagattgtagataagcagcattttaataagcaggctctttctgtcatctggcagaaaagtgatggtaacattgactagagtcgatggcatgggcatgaactaggttagt

SEQ ID NO: 292 - DNA, 599 bp, Homo sapiens
cttggcactaagttggcaaccaaggaaacgacaagagcattttcaacacatatttatatacagataccaaggcacactttccttttgtctgtgccactcttaaccttccattttatacttttgatgactaagccaaattctcattcaacccagtagtattatctctgtggtttgcaacaatacccttaacagtgtatataaaaaagagataggaatcattacagccacaaaagaaaggatgataggaacaactaggtcctagtgatgacatcctaatgggtggtcgagaactaaagttaytccaggggtctttggataacaccaagttgtagcctcagccagatacactcccttgccccagaacttccttccaaccccacccaatggctagatctccatgaagggaaactggattggaacaaaggcaacattcccaacacccagggttgatgggggattcacagtgtcctccctagtgaccccatgccttaagtctggcagccatgctagtcacttttaaatgactgacagagggctggcatttatttatttatttattcattttttactactgtggagtttaggggctctaaaaaaaa

SEQ ID NO: 293 - DNA, 599 bp, Homo sapiens
gactcagcaaggagaggcagactccatttgttgggaagaaagtaagggaagagaacaagtccgtacctggcaattcagagaattcttctggattttatccaagaacaccaacacactacctctatgactgcgagaaccaccgcattagtggcattggggtgccgcctaatgtagatacagtttaggtggcagcacccaagttcttttaaatacctgaaatcctttccaaacaagccccgactgtgaagactataagaaatacctaactactcaatgcccagacactgataacatctacargcatcaaggccatccaggaaaacatgacatcattaaacgaactaaataaggtgccaaggatcaatcctggagaaagagagatatgtgagctttcaggcagagatttcaaaagaattgttctgagaaaactcaaagaaatttgagctaacacagagaagaaattaataattctatcagataaatttaacagagattggaataatttaaaggattcaatcagaaattctggagccaaaaatgcaattggcatattgaagaatgcatcagagtcttacaatagcagaatcgattaagtggaa

SEQ ID NO: 294 - DNA, 599 bp, Homo sapiens
caacaaaattaaatatcccttcatgataaaaacacttaagaaacagggtatagaagaaacataacataataaaggccatctgacatactcatagctagtatcatattgaataggtaaaaactgatggccttccttctaagatctagaatatggcaagaacgcccactgtcatcactgttattcaacatagtactggatgtccaagctacagtaatcagacaagagaaagaaataaagaacatccaaattgaaaagaaaaagtcaaattattcttctttgcaaatgatatgatcttacatktgaaaaaatctaaagttttcaccaaaagactatcagaactgataaattcagtaaagtttcaggatacaaaataaacctacaaaaatcagaagtatttctatatcacagcagtcaacaacctaaaaagaatttaagaaagtaatcccatttacaatatctacaaataaaataaaatgcctgaaaagtaaccaaagaagtgaaagctcactacaataaaatctctaaaacattgatgcaataaatagaagaagacacacacaaaaattgaaaggtatccaatgtccacagattggaaaaat

SEQ ID NO: 295 - DNA, 599 bp, Homo sapiens
aaaactcttttgtctgaaatccctaacatctaaatttctgaaaataacaccacaaggtacttgcatcatgttacgcgcaattattactttgttattgcctttgtgtagaagaaaattattttcagttgaatctccaaactagataatgacaaatctggaattcgatacaatcaaggcttctaaaagtgaattttaaggtgttaccaataaagttcgactttgccattcagcccaaggcatttggctgaaaattcagacgagtggattggccacatgatacagcaaccacaaaaacttcascttgccagcattggcaatacttctagctgatgacatttcaggaacttttaatgaattgaagctgcatatgcctgaagcccctgcagccactgaccggttagaaaataattatgcaaatagtaagataagaagaaaattatgtaagtgtgttactgttttatcaccagtattgttttcaccaaacttgtggtctgtatatgaatgcatgcagaatggatttccttgtgctacaaacaacatagaaacatggcacagaagatgggaaaatgcaataaggaatgcccatgttggtatgtggg

SEQ ID NO: 296 - DNA, 599 bp, Homo sapiens
tcccaccagtgccataatagtttacaggtgccatggcaacgtcaggaagttaccccacatggtctaaaaaggggaggcatgaataaaccaccccttgtttagcataccatcaagaaatacccattaaaaatgggcaaccagcagcccttggggctgctctgtctatgaagtagccattctttcattcctttactttcttaataaacctgctttcactttactctttgaactcactctgaattgtttcttgtgtgagatccaagggccctctcttggggtctgaattggaacctcttttcrgtaacactttcacattcatacactatgtggcgctgctcttttagcaattattccatgattccccccacataccaacatgggcattccttattgcattttcccatcttctgtgccatgtttctatgttgtttgtagcacaaggaaatccattctgcatgcattcatatacagaccacaagtttggtgaaaacaatactggtgataaaacagtaacacacttacataattttcttcttatcttactatttgcataattattttctaaccggtcagtggctgcaggggcttcaggcatatgc

SEQ ID NO: 297 - DNA, 599 bp, Homo sapiens
tgtcagcattttttttcctaattctttatgctctatatgttatcttcacataatttccaatactggaggtataaattgtctaaagacttttagagaattataattagttttatgcatgtttggcaaatgtgacaatgaaattgcattatcacaacactgactttgtgtgtaagcattgtgcacatacgcaaaaacgaaactctctcagtacatgaagagacgtgctttttgtgaaagacaaaattccttgagatcttggctcttagggtgactggtgcaattgtgatgcagtagtgaccsattgtggtttttgactaatctcatcaaaggacttaggttgtccttctaggtacctcagatgactgcagttataaagctgggtgcacacaattactaaccacagtaatatgcatttatacattttgctttttgaccaattttttattaatatagtttatccactcataactgttatgttatctgttgttagcctacctagatgtttacgcttacaaaaatatgtatgttgtattgcctattttattgtataaagtggcccatgatgtgttccatcatgtttttatgtttctcaaacaaat

SEQ ID NO: 298 - DNA, 599 bp, Homo sapiens
gtacatgaagagacgtgctttttgtgaaagacaaaattccttgagatcttggctcttagggtgactggtgcaattgtgatgcagtagtgacccattgtggtttttgactaatctcatcaaaggacttaggttgtccttctaggtacctcagatgactgcagttataaagctgggtgcacacaattactaaccacagtaatatgcatttatacattttgctttttgaccaattttttattaatatagtttatccactcataactgttatgttatctgttgttagcctacctagatgtttaygcttacaaaaatatgtatgttgtattgcctattttattgtataaagtggcccatgatgtgttccatcatgtttttatgtttctcaaacaaatttccttttaaaaatgtaaataaatacatctttaaaatttttatttcttcagaattatattttttgaattttgatgtgtcaggattttaacattcaggattacggcatcagggttgtgtctttcaggattgtgactggcttttaaacattaaaatatatgaacatatataacctactacagtgccaagaaacagtatatttttcataa

SEQ ID NO: 299 - DNA, 599 bp, Homo sapiens
ttaacattcaggattacggcatcagggttgtgtctttcaggattgtgactggcttttaaacattaaaatatatgaacatatataacctactacagtgccaagaaacagtatatttttcataaatactactttccttccctttatagcaatataacttaattttgtgcttaatttgcaggatttatgccaagctgctttttataattctgtagcaaaaatatgtgaaatgcactgtggtgtgtattctgtcaataatatgggtttcctttaaggtatagttgaaagaatatgacagaactmctgggtcagttgccaaacagcaaaagtttaaacaatattgagcttccattaaaggtgagcaatgcaatatctggggtaaaacactagattttccaccacacgttttaaaatatatatggttaaaaaaatagctaaaatatataaaaataaatacttccaaacaattcaggaatggtggaagtctaattcagttagacaatgttatcattttgagtgaattttctagtaaaagactaatgttctcaagccaacactgagtcttatctgtttctattatgattacttctaggctatgtagc

SEQ ID NO: 300 - DNA, 599 bp, Homo sapiens
ctcaagccaacactgagtcttatctgtttctattatgattacttctaggctatgtagcattaaaaaaaagtcttcttcacatggatctggggcaagggaaggttgcctccaaaagagataggcaactggcattgaaagcaggggaaacagaggagtacaagaggtctcatattttccaatcagaagtaaccggcataattccaatatgagccttaaacagtaattcatagagacagacaaaattaccagcatcaatttctcattaaactgctgctcacatgagcaactatataaagtgtrtttctacagtgagtagcagcaggaatatatgcattttatgatgggaaattatttttctcatgagtgactggagaggtcagctaatcaaacataaattaatacagttgatgaaagagttgagggtcttacaattgttctgtgagacaatggattatccccaatggcttaaccatctactaacataatactatattattaggttactttttgatactatacagtatattttataagagtaatctagtcttttaaaaaaactaaaaaggaaaccttgaaagatatcgcacagaaggatctta

SEQ ID NO: 301 - DNA, 599 bp, Homo sapiens
attatgattacttctaggctatgtagcattaaaaaaaagtcttcttcacatggatctggggcaagggaaggttgcctccaaaagagataggcaactggcattgaaagcaggggaaacagaggagtacaagaggtctcatattttccaatcagaagtaaccggcataattccaatatgagccttaaacagtaattcatagagacagacaaaattaccagcatcaatttctcattaaactgctgctcacatgagcaactatataaagtgtgtttctacagtgagtagcagcaggaatatatkcattttatgatgggaaattatttttctcatgagtgactggagaggtcagctaatcaaacataaattaatacagttgatgaaagagttgagggtcttacaattgttctgtgagacaatggattatccccaatggcttaaccatctactaacataatactatattattaggttactttttgatactatacagtatattttataagagtaatctagtcttttaaaaaaactaaaaaggaaaccttgaaagatatcgcacagaaggatcttaaaacaaccacaaaacttacttaaattatcca

SEQ ID NO: 302 - DNA, 599 bp, Homo sapiens
cataaattaatacagttgatgaaagagttgagggtcttacaattgttctgtgagacaatggattatccccaatggcttaaccatctactaacataatactatattattaggttactttttgatactatacagtatattttataagagtaatctagtcttttaaaaaaactaaaaaggaaaccttgaaagatatcgcacagaaggatcttaaaacaaccacaaaacttacttaaattatccacataattccactaactctcacgtttacattgactaggatatgtgtgtacatgtatcttsaagtcaaagacaatttatgtcatgtaagaatgcaaaggtgaatcctgtgtttagacacatttccacatggttccccctactgcaataagttacaagtaaatgttttgttttacaaaaaaacaaggataaacgtaacaaaaaaaaattgtgtagaaaagttattgctagccccctaaaaaataaaatggtaaaatattttcaaagatagcactaagttaaaaagaaaattttttacattgacaaacatatgcgtttcttcacttgcaacgattggaacaccctatctacagtagaatcta

SEQ ID NO: 303 - DNA, 599 bp, Homo sapiens
tacagataggaagaaataatgatcagccagtcttgggttcatcaaggctagcatgagagttgaggtaaatttgaagtataaattgaagtctgcttgctggggctccaaatgttattatagcctgagatattgatctattaaagaaatcaagtgcttcgttcacttttgaaaatgaaatattaattagggttcacgaatttcctttcagtgggtagatattaaagtgttcattgccatcactagtgctgaattttaatcagtattttagaaacgatatgtgattatctaaagaaacattartggagtatttcttgcagtgtcctttaatagcaggcttgatatggaaggcctcacagcaaggacaaaaattagggagtaaaatatgctttttaaaatgtctctaccacatgtggcgaattactccaggtaaatgtgatctgacctgttattgtgtgtactatgtttggtatatttcccaacaatttcacagtaccttacattttaaacgccttttaaaatacatcttaataaataagaaattgatggaaaaatttataatttttaactacgtatgaaattttgggttgacaatttatgtt

SEQ ID NO: 304 - DNA, 599 bp, Homo sapiens
cagacttcaatttatacttcaaatttacctcaactctcatgctagccttgatgaacccaagactggctgatcattatttcttcctatctgtaaatagcatctgtggtctattccacagcatttaacacttattctctccagcactgtttcctactttcttatatgcatcaatctcttctctttagtggaaatatataaactatttatgaaaagattagtatgatattaggaatggaataattaatgatagaatttattgatagattagaaaaacatggattacgtgttataatgaaaacragaagcatcaattgcttattttatgttagaaactgtcctgaaatctggaataaaaagaaaaatcacatgtagtcctttctataaaggagatcacaggatgtggagtagtcaggattctagatacacgaatatgtagacagaaatcatagtgattgacagatacatatgatgataattactataatagaaaaacagtattttaggaagtaatgtggaagacccaaggagctaatgaaaggaataaaaccactcatcatagagaagcctatttattaattgtaaactgttgcaaggtcaag

SEQ ID NO: 305 - DNA, 599 bp, Homo sapiens
taagttgaacataatattgaattcattataagatatttgtaaggtattctggcctaaaaaaatttattatgaaatacagtaatcaaaatcaaaactggttcatccacttaatgaatgaatattgaccatgtgctccgagccagacaatgtgctaagttgttgagagagaaaagtgaacaagagagacatggtcctgtcctcatagcacttacagtatttccttttgaacagtgttgtatttattatatttagctcaatataaatctaaaattttgaacgattatatttatactttcctgrcactaaacatgcatgtgtattgcaggaaaatgtataaaatagaaacatggtttaggaaactgtaaaactatcacttcaatacttaatacttttatttttctgtacaccttattacttaaatgtaaataacagatttttaaaacctgagatctcattccatataaaatctgtatcatgttttcatgtagcttgatgttttaactattcttacatcaatcttcacaagcataattattaatattttcatattctcccaattcatttaacaaatagttattcaaggcatactctgtccattc

SEQ ID NO: 306 - DNA, 599 bp, Homo sapiens
acttatctctaatgttactgtagatatatatatattttttctatttcctgtctcattgcattaggtagaacttctaaaacaatgttcatagtagcggttatccatgctggcctcatcttaattttaataaaaattactgaaatgtttcacttttaactacagtggtagctgttgacttaaatatgcagtttttactgaagttaaagaaaaattctgttctaagctcgagtatatttttaccaaaaacaggtattagattttcctaaatgtctttttaagcatttactaagaactccacartacctcttacttttacttattgatagaatatattaatcattttcctaaaattaaattcccatttgcatatactaaatgaattatttattaaaatttatgtagttcaatatgctgctggaatttatttgctgctattttattttggatgtttttgtacctatattcctacatgagatggttctgtagttttgtttttaagtgctatctgtatgtgcttttgggattagggttttcagagttttatgaaatacatttggaagaatttagcattttcctgtgatctactgcaatttaaaatg

SEQ ID NO: 307 - DNA, 599 bp, Homo sapiens
ttgtttttaagtgctatctgtatgtgcttttgggattagggttttcagagttttatgaaatacatttggaagaatttagcattttcctgtgatctactgcaatttaaaatgctaatggtcttcattgatcctaattactgtgtgaacttaagtgcatcttgccatttatagatggaaaatgtctgaaactatttgttataatagtgaaattctatgtggcctagttccatggtagatagaaaagaattgggagtaggattaacctaatttttcttacacattcctaagaatcttaaaggytatcagagtactgagatttgtcatcaaattgagacgcaaaacattattttgtatggcacagagctcagtctcagaaaactacgcttactgcagtcatatatttacgtaaaattttttatttacattgaatagaaggtttatctgtaatgtatacccaaacagacatcaaataagacataattaacacagtgtagattttacacctgcctttccctgccaacataatatttgtttttattattttatgtttttttgtcactgtcctcaacattgtaacacttccagtctagagttatata

SEQ ID NO: 308 - DNA, 599 bp, Homo sapiens
aaaattttttatttacattgaatagaaggtttatctgtaatgtatacccaaacagacatcaaataagacataattaacacagtgtagattttacacctgcctttccctgccaacataatatttgtttttattattttatgtttttttgtcactgtcctcaacattgtaacacttccagtctagagttatatacagtgaatttatttttcagttttaaaaataatggttttaccatattcgttttaaacaactaatatgggttgtatatcttatagttgttcctcagcattaattaatctscagaagttaaggctgtgaatatataattaatgaacaatgtgaaggtgagatgtcatttaagcagttggcagattgtctttaaaattaataatctgtttctccaactgtttgcacttatttcaaggtgctactttcttcttgatgcctttttttcacttttttcctgttttattgatatgtaatcttttgcacatttatgaggtgtacatgagtgtttggtacatgcatagaatatacaatgatcaagccagggtatctagggtatcgatcaccttgagtgtttatcatttttaaatgtt

SEQ ID NO: 309 - DNA, 599 bp, Homo sapiens
atgatcatccctagtctctcccacccacccttcccagtctctggtatttatcattctattctctatgtccatgagatcaagtcacatgtggaggataaaaaacttagtgactttaagtcttaagtaactattcatgggataggctctgattgcgtgcctggctatggcataaaaaacttaattagaaaagaggagacatggtgctcttaaatttatgagaatacattttcaaagtttaaactggtgactttcaaacttctgaggaaaggaaaatactgccttctttgtatcgaaagtcaygggttgctcgtacatatgcgtacgtacatgactgatgtcacttggctcaatgaacaatgctaagttatcaagcatccatgagaactgcatttgtggaattcattcagttattttcgttcgtatatatttctttggcattttctccaattcttaaaaagtagaatagttttaattttcttacattggaacttctctaacttggtcaacatgattaattccttttatgtcttttgaacctcagtttctttatacgtaaagaggttcagctggattagatgttctctgagatctgacccctg

SEQ ID NO: 310 - DNA, 599 bp, Homo sapiens
ccacccacccttcccagtctctggtatttatcattctattctctatgtccatgagatcaagtcacatgtggaggataaaaaacttagtgactttaagtcttaagtaactattcatgggataggctctgattgcgtgcctggctatggcataaaaaacttaattagaaaagaggagacatggtgctcttaaatttatgagaatacattttcaaagtttaaactggtgactttcaaacttctgaggaaaggaaaatactgccttctttgtatcgaaagtcatgggttgctcgtacatatgcrtacgtacatgactgatgtcacttggctcaatgaacaatgctaagttatcaagcatccatgagaactgcatttgtggaattcattcagttattttcgttcgtatatatttctttggcattttctccaattcttaaaaagtagaatagttttaattttcttacattggaacttctctaacttggtcaacatgattaattccttttatgtcttttgaacctcagtttctttatacgtaaagaggttcagctggattagatgttctctgagatctgacccctgtgcaacatactacaatttgt

SEQ ID NO: 311 - DNA, 599 bp, Homo sapiens
ctgaggaaaggaaaatactgccttctttgtatcgaaagtcatgggttgctcgtacatatgcgtacgtacatgactgatgtcacttggctcaatgaacaatgctaagttatcaagcatccatgagaactgcatttgtggaattcattcagttattttcgttcgtatatatttctttggcattttctccaattcttaaaaagtagaatagttttaattttcttacattggaacttctctaacttggtcaacatgattaattccttttatgtcttttgaacctcagtttctttatacgtaaakaggttcagctggattagatgttctctgagatctgacccctgtgcaacatactacaatttgtagttagaagatattggcactgcagatctcattaaggacctgaataagacttcaagtttatgtatttgcctgcatttatatagatatatattttacataactaattcatttaaaatctctatgagtttattcatatggaaaataacaatagtaaaaaagactaccttcatagagttgtgaggaaattaaataacatttataagcactttcacagtacccagcccatgatgagtgctcaa

SEQ ID NO: 312 - DNA, 599 bp, Homo sapiens
attcattcagttattttcgttcgtatatatttctttggcattttctccaattcttaaaaagtagaatagttttaattttcttacattggaacttctctaacttggtcaacatgattaattccttttatgtcttttgaacctcagtttctttatacgtaaagaggttcagctggattagatgttctctgagatctgacccctgtgcaacatactacaatttgtagttagaagatattggcactgcagatctcattaaggacctgaataagacttcaagtttatgtatttgcctgcatttayatagatatatattttacataactaattcatttaaaatctctatgagtttattcatatggaaaataacaatagtaaaaaagactaccttcatagagttgtgaggaaattaaataacatttataagcactttcacagtacccagcccatgatgagtgctcaataaatgttgttatattaatttttattcttccttggcatcagtggcaatgtttctaatcaagtactaatatcattaagggcagtgtattctatctttttttaaaaaagaaactacagtcataatctgaatcattattttt

SEQ ID NO: 313 - DNA, 599 bp, Homo sapiens
gtcataatctgaatcattattttttagttaaattgtattcatctgtcatacaatttctcatgtgcacaattttcagaaacattttaatgtttctgaacaattccttattttaatctctacatctttatagtaaaaatgttaagcattgtgtgaccaattttgaataagtcattttgatactcatgattttgtctcgtaatagctaaaataatgagtgctgaattagttaagtggtttttcagtgggttgatagattatttaatgagttggcattaagcaaacaatgtccatatagataasaaattctgtaaaatgctctaactttagctattaaaattatgtcaaattttcgaataaagagatagtggcatgcaaatcaaattcacaggtatcacagagctggatataaatagcaggaaattaatagattagaataataaggcacctcatcatgctataaaaattgacaacatttagcaactaaaatataaaagggaataatcaaacaccaaaatcagaaatataaggtaaaacaagttgatacaagagatctctctaatcaactgcatgagtaaagaattaaggattaaatttaccat

SEQ ID NO: 314 - DNA, 599 bp, Homo sapiens
catgattttgtctcgtaatagctaaaataatgagtgctgaattagttaagtggtttttcagtgggttgatagattatttaatgagttggcattaagcaaacaatgtccatatagataagaaattctgtaaaatgctctaactttagctattaaaattatgtcaaattttcgaataaagagatagtggcatgcaaatcaaattcacaggtatcacagagctggatataaatagcaggaaattaatagattagaataataaggcacctcatcatgctataaaaattgacaacatttagcaastaaaatataaaagggaataatcaaacaccaaaatcagaaatataaggtaaaacaagttgatacaagagatctctctaatcaactgcatgagtaaagaattaaggattaaatttaccataatctcaatggatgaggaagctgtcaaaaaatacaacacaagcttaggctttaggttagttgaaaaatatgctatagaagaatgatgctggaattcccatattattctttgctgggaagaacacatttgaagaatgggatacacttctgggtacttcaatttcaatacaacattaaccagt

SEQ ID NO: 315 - DNA, 599 bp, Homo sapiens
agtaactggattgggaaaggtgacagccacagtgttgtcacagttggggctgttcttatcatttgtgatgctatacatgacgtgatggagcagaagtcactgaaaggctaaatgtgcttgtatggcagaacattaaaagctgcccataaaatgagaaaaaaaaatccctgcttcattgcctggacctgatgggctcaaatattagctggagaactatataagtaactaattttttaaagagtgccttggagatattgttgaggacattgtgaaatacagtgtttttgtcccaatatgaarccccttcagagaaatttctttattattgtagacaagaaagttttgatctcccatcaggggaactttttgttttaatggacaataaaaagtctataacattatatttccctttttacttctattgtttgaaaacagaattcactcagtcagcgattcagtgaatatttatcatatttatttattatattttcctattatatttcacagactattctaggtactggcaatgcaatagtgaaaatgataggtaaggaactttctctgtggaacatccattcttgtgggcatggagggtggaa

SEQ ID NO: 316 - DNA, 599 bp, Homo sapiens
caatatgaagccccttcagagaaatttctttattattgtagacaagaaagttttgatctcccatcaggggaactttttgttttaatggacaataaaaagtctataacattatatttccctttttacttctattgtttgaaaacagaattcactcagtcagcgattcagtgaatatttatcatatttatttattatattttcctattatatttcacagactattctaggtactggcaatgcaatagtgaaaatgataggtaaggaactttctctgtggaacatccattcttgtgggcatgragggtggaaagaaagatataaaataagtaatgaaacacatgggcaaaatattttcagtagagagagatttacaaagataattaaataagtcaacatgattgaaagaatataccatttaaagtaggcattagttgggcgcgatggctcacgtctgtaatcgcagcactttgggaggccgaggcgggtggatcacctgaagtcaggaggttgactccagcctgaccaatatgatgaaacctcatctctactaaaattacaaaaattagccaggcgtggtggcgggcacctgtagtcccagc

SEQ ID NO: 317 - DNA, 599 bp, Homo sapiens
tgactccagcctgaccaatatgatgaaacctcatctctactaaaattacaaaaattagccaggcgtggtggcgggcacctgtagtcccagctagctactcgggaggctgaaacaggagaattccttgtacccgggaggtggaggttgcagtgagcccagattgagccattgcactccagcctgggagacagaaagagactccatctcaaataaaataaaataaaataaaataaaataaaataaaataaaataaaataaaataaataaaataaaataaaataaaataaaataaaataaaawgtacgtattagagttgagtcctgactaattcagacaactgccaaagctcaatatcagagctttaaagcaaagggaacagcaagtacaaatgtcctcaggaaggcaagaaccttggctgttggaaaataggagaaggaggctacaaaagtgattggagcttggtgaataaacagggagatcagagagatgggcaggaatcaagtgtgggccccactcattaggattttactccatgtgtaaagggaagccattagagtatctttaacaggggacagcatgatctgtttcttaactttaaa

SEQ ID NO: 318 - DNA, 599 bp, Homo sapiens
atgatttgcaaatattttctcccattttgtgtatcaagggtaatgctggcctcacaaaatgagttcagcagtgttctctcctctttaaattttttggagaagcttgagaaagatttatgttagttcctctttaaatgtttattagaattcatctgtgaagccatcaagtccagggcttttctttgtcagaagattttttattactgattcaatctctttactaattgcaggcttattcagattttctattggttcatagtttcgccttggtaggctttgtgtttctaggaatttgtcctstttatgttatccaatttgttggcatacaattggttgtcatattctcttacaattctttttatttttgtagaatggtaataatgtctcactttaatttctgattttagtaattgagtcttttttttccttaggtcatctagctaatgtttagtcaattttgttgatttttttgaggaacaactttttgtttcattgattttctctgttatttttctattctgtattttttaaatctgtgctctaatctttattttttcttccttcagtcagctttgggtttaattttggtttgggggtaa

SEQ ID NO: 319 - DNA, 599 bp, Homo sapiens
tatttgctaataattttattaaggatctggtgtatgtaatagtcactttactcttgctgctttcaaaattctttgtctttgtctttcaacagtttagctataatgtatctcagtgtcagtctctttgagtttattctacttactttgttgagctccttggatatggacatttatgtctttaatcaatttggggaagtttttagttattatttctttaaatattctctctgttcatttttctcttcttctggagcaccagcaatgtgtatgctggtccacccaatggtgtcacacaggtcmctcaggctgtgatagcttttttccaatcttttgttctttctgtttttcggacaataattcccaccatcctatcttcaagttcactaactctttcttctgcctgctcaaatttgtctttgaattcctgtagtaaatttttttatgtcagttattgaaatttcctgtttcagaatttatttttagtttattcttagatagtccatctctttattgatatttccattgtgtctatacatcattttctttactttctccatatcttgtttagttctttgagcatttttaagacagttgtttta

SEQ ID NO: 320 - DNA, 599 bp, Homo sapiens
caattatagagttaaataagtactcagtggtataacctccttcctaatgtagaaatctcttccaagatgtcaccagcgctttaactaacttttgttcaaatgcctctactgtcatggaactcacagcctcattattaaatattttcttctttgttgtaattgttgaactaaaatcttccttctgataattcacaagatttgctttaccctgtgactgtcactttcaatgtttgcactttattaaaaaagaaaaacaactgtagaaaatgaggtgcaaattaagacgacatatttattttwaaaatgaagaaaaaccaagtacttgatgaaagaatagatcacttttaagagctaagaatgcttaggaaaagtcacaagatataatcagtcagttagagttttcttttaatttttttagactacaaattattcttcagaagatgaggatggatgtgcaatagggcttattgtctttttgcttttcatttttattgttgtgtgtatataacaccttccttatgaaaaaataagttttatgcagcaaatttctgaataaaaacaaaggaaagtttaataccttaggtaaattttaagctatc

SEQ ID NO: 321 - DNA, 599 bp, Homo sapiens
taaatataagcagattcctcctcctggtcaagtgatgtgggttagagacttctcaatctttccagtcctaaatgtatatctcatcattccagcgatgattccctccaacatggtatagggtttaaatcatgaaaaataattcctttcaatttagactctcatattttcattgaactatatttttccacgttccctaccctttgagtcttacagaagaaacctattaaaatatcacatcactatataagaggacagatattttaatttgcttggctatagtaatcactttactatgtatayttatatcaaagcaccatgttgtacacctaaaatatgtaaaatttgaaaaaaagttgaaaaatatcaaaataattatatcagtcaggcaatatttttagtgaaaagacattggctttttatttggcacaaagtgaacctcaactcaagcagttatttcatgcttattttctaagcagataagcttcaaacatattaaagtatctagttacgtagattctttactcttatttcttttaccaactgctctgttccttgagaaaaatatggaaattttaaaataaaagtttaagttgttagat

SEQ ID NO: 322 - DNA, 599 bp, Homo sapiens
tcctggtcaagtgatgtgggttagagacttctcaatctttccagtcctaaatgtatatctcatcattccagcgatgattccctccaacatggtatagggtttaaatcatgaaaaataattcctttcaatttagactctcatattttcattgaactatatttttccacgttccctaccctttgagtcttacagaagaaacctattaaaatatcacatcactatataagaggacagatattttaatttgcttggctatagtaatcactttactatgtatatttatatcaaagcaccatgttktacacctaaaatatgtaaaatttgaaaaaaagttgaaaaatatcaaaataattatatcagtcaggcaatatttttagtgaaaagacattggctttttatttggcacaaagtgaacctcaactcaagcagttatttcatgcttattttctaagcagataagcttcaaacatattaaagtatctagttacgtagattctttactcttatttcttttaccaactgctctgttccttgagaaaaatatggaaattttaaaataaaagtttaagttgttagatctcaactaggaaatccacaaa

SEQ ID NO: 323 - DNA, 599 bp, Homo sapiens
gttttcttattataatcaccacatacttattctacatgtattattttatgcatcaagtattattttatgaacttaaataagaaatactctaaataaatcagcaggaattagatacatattttcaccattagattatttagcaatgtataggaataagagaagtgataatatatgaaaatttcctctgtagaagtttaaaacttagaataaatttgtatttgtttggtatgctgaatgcatgatcccaccttgtggcagagaactgaaattgaggatcaggatgtctgcagatgccaaaaygtttatactctttgtgaccaggccattgtattttggcctgagagttttctagacatttcccaatcaccctgatgattggataccaaaataaaacacagtttaaatgagtgaatgcaatcatagcacaggagggggaagaaagaggaagagattttcttttcatcataatttttcttgtcaattaatataacaagtctcctaactcagacacatagtctcatgacaaattgaagaatatttattttaggttaaaatatttttaagattatcccttgtctcactgcaagagataaatggca

SEQ ID NO: 324 - DNA, 599 bp, Homo sapiens
cctttctggagatctgactgggcaggatctctctctctctttccctgctccctcccaccatctctcttctttaaggctcctatcatattatctatccctagtatcacagttctttgtttgttactgcccttcacaaagtttcattatttctcacattaattgctgaaaaagttgaaaaatgtgtgttggttgacatatgtatcactttccaaagaaatgtaagggtttttttttccttatttctctgaaggtttaaatagacagcatttgcatacaaaatatcacatatatgtgacatawagattttagtgtagaactagatttatgagattattttggcaaaattcaattttccatggaattttcattttaatacattcttgtggaagaagcatatattgatttaacaaatatcaacacctgtatctgttagtgtgattcttttttttccttcagatacttttatgtatctcatatctacttgattcaaagaaatgggacatggtaacttagattttctcattttaatgctaccaagtatgcatttttaaattttcttttctaccaaggaaaacctatatttcatattcatgctatgt

SEQ ID NO: 325 - DNA, 599 bp, Homo sapiens
aaaggaactttcttaaaaagaagagtcaaccatgaagctcttttcattataggtgaagcccttggaaatcatgctctaatttcttaaacacccaaaaactttttaaattcaaatttctggtagctagctggccaatatgtaactatatcaaggtctctagcattggcaaacaatattcaagactttgacagtgtattaaattagtttagttttttttttaaatagagtaataaaaaaaattccaaacaattagttgacactgagtaaagccttgcttgcgcatatgttttatttaggtcmgtattaacctgtaacttttttcaaaggtctgtacaaattcaaattagcatctactgatgcttaacttggttttccttatttcacacaaagtattttaaagctgaggaaagtgaggtataaacaggtaaaattactatcacaagatattcagctagttactaacagagcgtaggttcaaattcaagttttcaaaatccagtgtctttgtataataaattttgatacctccaaatgatgcttgatatatatatatatttaaagtcttcaaaagaatataattacttttatcttataaactt

SEQ ID NO: 326 - DNA, 599 bp, Homo sapiens
taggttcaaattcaagttttcaaaatccagtgtctttgtataataaattttgatacctccaaatgatgcttgatatatatatatatttaaagtcttcaaaagaatataattacttttatcttataaacttgttattgttaaatttttaccttcaatatagcattgtccatgtctaatgcatcccatctccccaaaaaatgcaaatacatttctctgccctatgatcagagatacttggtatacaaatctaataattatttttaattctctcaccttttagtctcaaaattatatgccatwttaacctatttttgatcattttaataactacctgaatgcactgatttgaatatgattcaataaaaaaaatcactatattttggctaatctgtaaactaaggccatcagcactgatgttttaaaaaatgtacagattgtgtcttcttaatgcccaatcagaagacaattatacttatttcctgacaaaatgtataaaaagacgagtgtcaagccacgttccatatatatcacagacaatctttaaatgtttttgagtcaaataacaatggaatagatgcttgaaaaaatgcataggtggc

SEQ ID NO: 327 - DNA, 599 bp, Homo sapiens
aacttgttattgttaaatttttaccttcaatatagcattgtccatgtctaatgcatcccatctccccaaaaaatgcaaatacatttctctgccctatgatcagagatacttggtatacaaatctaataattatttttaattctctcaccttttagtctcaaaattatatgccatattaacctatttttgatcattttaataactacctgaatgcactgatttgaatatgattcaataaaaaaaatcactatattttggctaatctgtaaactaaggccatcagcactgatgttttaaaaratgtacagattgtgtcttcttaatgcccaatcagaagacaattatacttatttcctgacaaaatgtataaaaagacgagtgtcaagccacgttccatatatatcacagacaatctttaaatgtttttgagtcaaataacaatggaatagatgcttgaaaaaatgcataggtggctaaataagtggctacaactatacaattcatcttagatgaagagagaactggttaacatattttaaattctgatttcattttataatgcacaacaattccctgatctcaaccctctggtccttcaa

SEQ ID NO: 328 - DNA, 599 bp, Homo sapiens
agccttgaaaaaattgtttcccaatataatcattgctattacctggaaaagtgaaatatttgcaatagttttactattctcctccttaccatgtgtctaggttacagtgaccccacgggaaagtatgaccctgctgttcgaagtcttcacaatttggctcatctattcctgaatggaacagggggacaaacccatttgtctccaaatgatcctatttttgtcctcctgcacaccttcacagatgcagtctttgatgaatggctgaggagatacaatgctggtaagacattttcatatgcyttttgcatgctcagctgggcggattgtttagatggcatagttatcagttcaagctgagcactcagcgcataaaaacactttcaaaataaggatagcatagctgtaatatcaagtcacttccagacattcaattctactttgaaaatgcaggcaagaagtctctccaaatagttattataggatgatatttttaaccatttgctgggactaaccaaatcatctctcctaatttttgctacgaaaaaatggctactatgtttcacaaattgttcttaccctcatcttccaacataaattct

SEQ ID NO: 329 - DNA, 599 bp, Homo sapiens
aaaaacactttcaaaataaggatagcatagctgtaatatcaagtcacttccagacattcaattctactttgaaaatgcaggcaagaagtctctccaaatagttattataggatgatatttttaaccatttgctgggactaaccaaatcatctctcctaatttttgctacgaaaaaatggctactatgtttcacaaattgttcttaccctcatcttccaacataaattctcattacttttaaatataaatgaggaaaaccttatagagctcttctttgatatcccaaaagactaaaaattktatacccataatccacagttcttatttttattacttaatggcccctcacaaatagaaattgtgttccacttgaaaagttaatgttggcttttatatcacactataggaaatctcttttaagttcatttcaatgcattctttaaaatactatttaaaaggcctctttctctgccttcctatgcagctgagaggctagcattatttttcaaacaataaaagtgcttccactggttaaaacatcgcaaattttcataggttgaaagcattctaaattttggcttttgtgcctttagctcata

SEQ ID NO: 330 - DNA, 599 bp, Homo sapiens
tgtcagcttttttaaaagtgaaacaataaatgagtgacttttgaaatacaatatgagggcaaatattttagccatacacctttaacaaatctttccaaaatgttcaatttacatactataaaaacaacatccttctctttgttttacagtcatgatgaccactttatgtttgtttaactagattacttttattacagtaatgagtaaactcaaattaataggtttaggaattaacaaaaccagcttttgttaaacatgcacttctgggaactacatggcaatgttcaagttgtatttatycagttattcacaatgccgtgggtgttagtaaactttttagaggaaatggagactattcagtaacctggcaagtctcttaagtggagttttctttcacctatgaaagaaaggaaagatattccttcaccttgtgtatcttggctgaaaatataattatttcacattgatagcaagttgttaaaggttctccagattttaacttaaaggtcattgatgaaccagaaaaataccttttgaacatttttttctgacattttctttgtttgcaatatgtgtagcataaggtttctcagtaactt

SEQ ID NO: 331 - DNA, 599 bp, Homo sapiens
tgtttaaatcttttccccaacaaaggcagtagggcatagtggaaaagtacaagacttatattctaaaatatccgggctccaaagccaactttattgcttaccaaccaagtgacctgggtaagtgactcagactcattgagtcatcattctctcactttcacaaaatgaaatggaaataacaatgactatcccaatagggtccactcattaaaagaaatcaggaagtggcttcaaggccatgtggccaatgtaaattaaatatgaggatttctgttaaaatagacatttctaaatttcatstgtccactttttggtgataactattttaatatttgtctttttatttttatcttcctttccaaataggtcgggagtttagtgtacctgagataattgccatagcagtagttggcgctttgttactggttgcactcatttttgggactgcttcttatctgattcgtgccagacgcagtatggatgaagctaaccagcctctcctcactgatcagtatcaatgctatgctgaagaatatgaaaaactccagaatcctaatcagtctgtggtctaacaaatgccctactctcttatgcattag

SEQ ID NO: 332 - DNA, 599 bp, Homo sapiens
gccatagcagtagttggcgctttgttactggttgcactcatttttgggactgcttcttatctgattcgtgccagacgcagtatggatgaagctaaccagcctctcctcactgatcagtatcaatgctatgctgaagaatatgaaaaactccagaatcctaatcagtctgtggtctaacaaatgccctactctcttatgcattagtatcacaaaaccacctggttgaatataatagattgagttattaactgtattttctttcactttattaccttctttctaatacaagcatatgttagmattaaagttctaggcatacttttcaaagctgggaagaccctttcagaatcttttcaatgggttttaattttcagttctatttaaaatggtgaatgacactaaactccatgatatttaaggatagtgtgaagatctttggcatgatttaaaggttgagtatgtgaagatataagtaagtgaactaccatgctttgtttacgtgtaaaggaaaataatgtttgatagtaaatgtccacttaaaatacatgaatgggcatttctaaaatgttaaaacataaacacatttccattcatggata

SEQ ID NO: 333 - DNA, 599 bp, Homo sapiens
aatacaagcatatgttagcattaaagttctaggcatacttttcaaagctgggaagaccctttcagaatcttttcaatgggttttaattttcagttctatttaaaatggtgaatgacactaaactccatgatatttaaggatagtgtgaagatctttggcatgatttaaaggttgagtatgtgaagatataagtaagtgaactaccatgctttgtttacgtgtaaaggaaaataatgtttgatagtaaatgtccacttaaaatacatgaatgggcatttctaaaatgttaaaacataaacwcatttccattcatggatatttgtcaacagatttaaagaaaaccacagttattaattaaagaaaattaattatgtgtagttataaaccaatgaaattttgattaaccttttcaaattaatgttccagtttgaagaccaatcaaatatattatttagtcaacatatactatttagtctcaggttcaaggctacaacaaaaatcaccatctttgtcaaactttggagagggaaaatcttcactttcttaagcaacaatggatattgcctgtgtttgccactgtgtttccctgcctctcaatt

SEQ ID NO: 334 - DNA, 599 bp, Homo sapiens
atttagtcaacatatactatttagtctcaggttcaaggctacaacaaaaatcaccatctttgtcaaactttggagagggaaaatcttcactttcttaagcaacaatggatattgcctgtgtttgccactgtgtttccctgcctctcaattcgctgaaaaaggaactacctatccttacatttcacctactaatgtctcttctaacatcttagaggtccatggagaaggcatatggagaacatgttttatactgctctataaatagtattccaatcactgtgcttaatttaaatagcattmtcttatcatttatcagccttttatgtattttccaagtaaaatattaacatattatttcattggtcttcttttttatctggttctatatgaatgctattttttcccttctcttctaacatgaaatatattttctctttttgatcttgtgctatgaaacaatcttccaaagaactgtataaggtggtcataagtgaatattttaattaaaattggtaaaaataaataataacagtaataatcatgcactatagaaaatggctaaactgagattctaaattctacaaacagaaacaagttta

SEQ ID NO: 335 - DNA, 599 bp, Homo sapiens
atctttgtcaaactttggagagggaaaatcttcactttcttaagcaacaatggatattgcctgtgtttgccactgtgtttccctgcctctcaattcgctgaaaaaggaactacctatccttacatttcacctactaatgtctcttctaacatcttagaggtccatggagaaggcatatggagaacatgttttatactgctctataaatagtattccaatcactgtgcttaatttaaatagcattatcttatcatttatcagccttttatgtattttccaagtaaaatattaacatattayttcattggtcttcttttttatctggttctatatgaatgctattttttcccttctcttctaacatgaaatatattttctctttttgatcttgtgctatgaaacaatcttccaaagaactgtataaggtggtcataagtgaatattttaattaaaattggtaaaaataaataataacagtaataatcatgcactatagaaaatggctaaactgagattctaaattctacaaacagaaacaagtttaagttatgtatccctgattggttactgggttttcctatattcaaaaatattaaggc

SEQ ID NO: 336 - DNA, 599 bp, Homo sapiens
ttgtacttcaaggactgccaatggatttaccaaatatagaagcagagaagggaattttattcaaaagtgacagcatggacaagggcacagaagtttaaaagagcatggaactttttaaaatataaaataggtcagtgtgattacagtatagtttttatggaggggagtaaaaagataaattggaaataaaaccagagtgtcctatatgcaattcttaatacttggaattaattccatgggaaataggaaacagggagagtcataatcaagctgtatatttataaatatagtagtgatggwgctgttcacaaataacgttgatgagaaataatttggaatggttaaaagcaaagaaactcattaatatggaatagattgtgggagagaggagaaagccttgtttcaaagcattaattctaaataatcataattataagataaagttgtacacatctataaataaataaatacagcttatattataaatactatataatattgaaattataagtataatataaatatagctttaagtaaatcattacaaaaatgtcagttaacaatggaagactgtgagagagaggattaaaacaagagca

SEQ ID NO: 337 - DNA, 599 bp, Homo sapiens
cattttatgtcaagtgaaggagacagaagaatgtaaaagttatatctccataggaggtgtgtggaatgtaaaatatcttgtgtgtgggcagtaaataccttggaggtgtgtggacagtaaaataccttgtcgaaaggatatattcagttctattagattagaggaattcatccaaaaaggattataaaacaccaaacaaattaaaaagtgggataattattaattctagggaaacagccatttgtttcagaaaagaaagtcattgtagtgtactatgtagtatgctgtaaacaacaaaawttccatctaaatagtaaaagaaattgattaaataataacaaaaatatgatataactaaattgaaaaatgtaagcaaggatgaagtaaaatatgttggtgagcaaggctatgataaaagctgagtcctcatcatccaaggtagaatatcaaaacttactgtctctaaattttttaaaaagataaaaatacatttaaaaacagaggcaaaattagaagaaataatttgaagagctgaaaaagttgtatgaaaaagtgtcatctgtgtataggaaaggctggggcaggagaccttgatcatt

SEQ ID NO: 338 - DNA, 599 bp, Homo sapiens
acagtaaaataccttgtcgaaaggatatattcagttctattagattagaggaattcatccaaaaaggattataaaacaccaaacaaattaaaaagtgggataattattaattctagggaaacagccatttgtttcagaaaagaaagtcattgtagtgtactatgtagtatgctgtaaacaacaaaatttccatctaaatagtaaaagaaattgattaaataataacaaaaatatgatataactaaattgaaaaatgtaagcaaggatgaagtaaaatatgttggtgagcaaggctatgawaaaagctgagtcctcatcatccaaggtagaatatcaaaacttactgtctctaaattttttaaaaagataaaaatacatttaaaaacagaggcaaaattagaagaaataatttgaagagctgaaaaagttgtatgaaaaagtgtcatctgtgtataggaaaggctggggcaggagaccttgatcattctttatcagccttgacttttttaaataaaaatttttaaaaagcaggtgatgcttctggaaacctcggtgaagaatgatttatttcagaatgtataacactaaagcagaagttc

SEQ ID NO: 339 - DNA, 599 bp, Homo sapiens
aagacacctgttattccttttctggttttaaaaaacgttcatgtgattcttgaaagcagaagaaatgaaattttcatttgagtagaaatgggtttgtgtgattaataatctccacatggctgaaatttccaactaaataacaatgacaaaaatgtatcttctttatcgactaaaactctcttgtatacattatcccccttcatcatgatcactgatcataaagcccttccgggccacaaaatgaagagtgagtggcatatcacccaaatggttccacgtacaatagatgattatgccagraccaaagacattgataatttctgacaagggtgaaagggctagccagctctgggaatcgtctgcattcagctgctgagaatacaaatactgaaatcacagttaactgtgtacatgtacacttaaagatttcaaaactagcctgagaagcctaaaaaaattattttaacttaccttgaaaaaattataatttagagaaaaattgcaagaataaaataaattcccatggacactgcacctagatttaacaattgtcagtattttgcacatttgcttttttactctatctccatgcatgccta

SEQ ID NO: 340 - DNA, 599 bp, Homo sapiens
ataaaataaattcccatggacactgcacctagatttaacaattgtcagtattttgcacatttgcttttttactctatctccatgcatgcctatgtgcgtatcattattatgaacatttttcctgaacaaaattgaaagtaaatagcagatattatgaccttttatccctgttttagtatgtgcctcctaagagcaatggcattcccttacataatcacaatacaactgtcaggaaatttaaaatggatgcaatactatgttctaatatatagcccatattcaaattttccagttgtttcrgtagtatcctttggagtgattttttaaaataacaaactcaacattttgaagaagaaattaaaaaatggcacataaaaatataaacaacataagggaaacttaaagagtagaagaaaaggccaagagaggacattctatcaaatttcctttaggggaaataagtcaacatctgtttgtgataggcaacatgataagactaattctatattcaaacagccctagtttggcttcctgtctcttccatttattagcaatgtgtccttgagcaagttgcattacttctctaagcctgttttctt

SEQ ID NO: 341 - DNA, 599 bp, Homo sapiens
taagtcaacatctgtttgtgataggcaacatgataagactaattctatattcaaacagccctagtttggcttcctgtctcttccatttattagcaatgtgtccttgagcaagttgcattacttctctaagcctgttttctttatctgtaaaataaaggcggttcttacaaaatagtgatttttagaacatcagaagtgataatggtaagtatatctcccaatacctaaaatgtagtacttgcttaacaaattttaacttttattcctagaataatgaaaacttaactctttccagctacytccttaaaaaaatgatttggaaaaaaagaatttatctcttggttcccaagttaaatctcttggttcgtgataccatgttgcacacgggtttccatctgaacactctcatcccactgccattgaagggcatgtcacagctaattatctgtcatcacgccctgttgcataggcagtaccttgcctgctgctgctgggacctcagtgctgtctgttgattcatttgcattgcctcctgagtgtctctgctgtgtatggatcacacagaatgaagctcaggggccaactggcaggcccatctg

SEQ ID NO: 342 - DNA, 599 bp, Homo sapiens
ctcaagttgtatttatgtccctttaccatggaaagattgaaacccttcccaatacgtttagtgctccctgttcggtgtctgagtgactgtgatgtttcaatttaaactctaaaagaacatgtatttgaggcaaaatcagactatgcatatctagttctgattccccaaattagacttaaaatatataaataaattcttacagcatgtgtagacatagaacaagtcccaaactgttaggcatatatgtataataccataattttgttccaatacaaacttcattctttcaaataaactgcrtaatatatacttgtaaaaaaatctgccttcaatttattttaaaaaattatcttcaaactactgacttcagaaagtagctgtggccattcagaacagggcattactgagtgacaaggatactgtttaatcttggtatgaattcttatttcattaaaggatgaggtttcttaatttttccttctctttcctccatattactctataccttttactatgagatactcctatagaaactaaatttaaagtctacctagaaaagaaaaaaaaaaaaaaaagcattgtaaataagccccagccat

SEQ ID NO: 343 - DNA, 599 bp, Homo sapiens
agatttctggggctttgacagttttcaaaataaagcttggagaaaaattacaaaatagggcgtaatgaatttcttaaatacactgaaactatcggtgcaggcgtgtcactattactttcctggtttgatcactgggagtcatccagttgatacacagcctcctcaggaaacttaatttggagagaaaatagagaaggattctctatgactggaggaatgtggatgacagtgttttaatagaatacatgttatgataaatgtcagtcacacactcattaacatatttgtatcttgtgttaytttaaaaattctgtgttagtctaattaaacaaatctacgccattggtgataaaggtataatgcaagacttcctgccaatgagcattttaaattgagcccaattattgtaatgggtatcaataattatctacaaagtttattgaaattttagaatgttaatgtctgtccacaggacaaggtaacttatgatagccaataatgaatgatagggctttccaatgccaaaaaaagatggaaaaaacaatgtgtgtttgtagaaatatatcatagccatatcttgttctttctagtacaaaatc

SEQ ID NO: 344 - DNA, 599 bp, Homo sapiens
aaacacacattgttttttccatctttttttggcattggaaagccctatcattcattattggctatcataagttaccttgtcctgtggacagacattaacattctaaaatttcaataaactttgtagataattattgatacccattacaataattgggctcaatttaaaatgctcattggcaggaagtcttgcattatacctttatcaccaatggcgtagatttgtttaattagactaacacagaatttttaaaataacacaagatacaaatatgttaatgagtgtgtgactgacatttakcataacatgtattctattaaaacactgtcatccacattcctccagtcatagagaatccttctctattttctctccaaattaagtttcctgaggaggctgtgtatcaactggatgactcccagtgatcaaaccaggaaagtaatagtgacacgcctgcaccgatagtttcagtgtatttaagaaattcattacgccctattttgtaatttttctccaagctttattttgaaaactgtcaaagccccagaaatctgaaaaagcaaagcaaaacaatgaagacttgtatagactatttattt

SEQ ID NO: 345 - DNA, 599 bp, Homo sapiens
ctaaataaataccttgacatgtaatttaagcaaaacatgtttctgaggagatcatataaattattgttaatcaaaatctcaggtaatgtaaatagctctgtcagttttccatgaagccctcaatccaattcttatatgacacacaaggaatgtatggatagatgtatgtgtctaaattcctgctggtatgattgtagtcctcaaaaaacgctggagatctggatcaggtagataatcacatacagagcaatcaggaagcaaagccaagttgataggatctttgagatccttttcagagcycctacttcaaagccagcacaatccctaccctccgaagagccaatcagtgcactttacgtctcatatctctaccattactctgttacctgagcaaagtctgaaaaccaccactggaaaattcagaaatataattctaatgacctcacagataaacctctccaattatggcaaaatcaggtacctctctgatcacagacaggaacttagaaataactaaaaactaaaaaaaaaaaaaaaaaaaaaaattaccatgatggaagtgactctgccaactataatggatgaatatttattcattt

SEQ ID NO: 346 - DNA, 599 bp, Homo sapiens
acatgtttctgaggagatcatataaattattgttaatcaaaatctcaggtaatgtaaatagctctgtcagttttccatgaagccctcaatccaattcttatatgacacacaaggaatgtatggatagatgtatgtgtctaaattcctgctggtatgattgtagtcctcaaaaaacgctggagatctggatcaggtagataatcacatacagagcaatcaggaagcaaagccaagttgataggatctttgagatccttttcagagctcctacttcaaagccagcacaatccctaccctccraagagccaatcagtgcactttacgtctcatatctctaccattactctgttacctgagcaaagtctgaaaaccaccactggaaaattcagaaatataattctaatgacctcacagataaacctctccaattatggcaaaatcaggtacctctctgatcacagacaggaacttagaaataactaaaaactaaaaaaaaaaaaaaaaaaaaaaattaccatgatggaagtgactctgccaactataatggatgaatatttattcatttcttagtttggatgggtgacaaattattagccttt

SEQ ID NO: 347 - DNA, 599 bp, Homo sapiens
tggcaaaatcaggtacctctctgatcacagacaggaacttagaaataactaaaaactaaaaaaaaaaaaaaaaaaaaaaattaccatgatggaagtgactctgccaactataatggatgaatatttattcatttcttagtttggatgggtgacaaattattagcctttcttaggcaccaactcttagtctactcaagtttcttccataaccttatgactaacctaactgatcatgtcactctcatagttcaagattaaatttcatctatcagtgttctccagagaataaggttgggacastgtcaactttttttgagactttattttgtactttgtcaaaacattaaataattttaattctgcttaagtaaattttttttaagaatatctttgaagatctctagtgaaattattttataagttcaaagtactgttttagttttatatttgctactgtaacaaattacaacaaacctagtggcctaaaacaagacaaatttatttttttcacttctgaaagctgtttttacaaagtcaccgggctgacacccatgcatggtcaggcctgtactgtctcgggaggtctttttctttgcctt

SEQ ID NO: 348 - DNA, 599 bp, Homo sapiens
ttacaacaaacctagtggcctaaaacaagacaaatttatttttttcacttctgaaagctgtttttacaaagtcaccgggctgacacccatgcatggtcaggcctgtactgtctcgggaggtctttttctttgccttttctagcttctacagaaagtgtcataccttgcattccttggttcatggccctttctctatctccaaagccagtaggatcatatcttctctgactctgcttacatgtccttatgtcacatggccttctttgcctctgtcaaatctccctccatctttactttatkcgaaaatttgtgattgcatttaggacccacccaccaaataatccaggataatctcttcctttgaaagtccctaatttaatcacatcttcgaggtccactttatcacataaggtaacattcacaggttccaggtattaggacctgaacatcttcgggggtcatttttgagcttaccataaatactctctttgtgtgcccattttgagaaattggctcagttattaccaatagataaaaatgaggccagttccaaattcttcatttttcttcattaaactgctgtgaaaagtttattctta

SEQ ID NO: 349 - DNA, 599 bp, Homo sapiens
atggtcaggcctgtactgtctcgggaggtctttttctttgccttttctagcttctacagaaagtgtcataccttgcattccttggttcatggccctttctctatctccaaagccagtaggatcatatcttctctgactctgcttacatgtccttatgtcacatggccttctttgcctctgtcaaatctccctccatctttactttatgcgaaaatttgtgattgcatttaggacccacccaccaaataatccaggataatctcttcctttgaaagtccctaatttaatcacatcttcgargtccactttatcacataaggtaacattcacaggttccaggtattaggacctgaacatcttcgggggtcatttttgagcttaccataaatactctctttgtgtgcccattttgagaaattggctcagttattaccaatagataaaaatgaggccagttccaaattcttcatttttcttcattaaactgctgtgaaaagtttattcttagacagttttctggcaatgatagaagacactgaatcaagactgtaaaactgactgaagaattctgcttttacctcataaatcttcccccaaat

SEQ ID NO: 350 - DNA, 599 bp, Homo sapiens
cattccttggttcatggccctttctctatctccaaagccagtaggatcatatcttctctgactctgcttacatgtccttatgtcacatggccttctttgcctctgtcaaatctccctccatctttactttatgcgaaaatttgtgattgcatttaggacccacccaccaaataatccaggataatctcttcctttgaaagtccctaatttaatcacatcttcgaggtccactttatcacataaggtaacattcacaggttccaggtattaggacctgaacatcttcgggggtcatttttsagcttaccataaatactctctttgtgtgcccattttgagaaattggctcagttattaccaatagataaaaatgaggccagttccaaattcttcatttttcttcattaaactgctgtgaaaagtttattcttagacagttttctggcaatgatagaagacactgaatcaagactgtaaaactgactgaagaattctgcttttacctcataaatcttcccccaaatttcctgctctccaattatttttcctcctctttacatggaatctttttatgctatatttctacttagaatacttct

SEQ ID NO: 351 - DNA, 599 bp, Homo sapiens
ctcagttattaccaatagataaaaatgaggccagttccaaattcttcatttttcttcattaaactgctgtgaaaagtttattcttagacagttttctggcaatgatagaagacactgaatcaagactgtaaaactgactgaagaattctgcttttacctcataaatcttcccccaaatttcctgctctccaattatttttcctcctctttacatggaatctttttatgctatatttctacttagaatacttctatgatagacattttggtttcaccaattagcagagaatttcaaattcstcctctaaacctagtttgtttattggtttgctttccttttctttacatgttcatgtaattaagatggctttagtagtcacagcttgcacaagaaaggttgactcacctgcccgtgtgaaaacatctttcagatttaagaagagtcccaagggagcatcccgagtttattgctctttgcaggctataagctctccttaagtgatctcaaccattttcatacttttaattaccttccctaggctaataattcctgaatctctatatttagccttagacactgccatgagcttcaatttcac

SEQ ID NO: 352 - DNA, 599 bp, Homo sapiens
aaactgactgaagaattctgcttttacctcataaatcttcccccaaatttcctgctctccaattatttttcctcctctttacatggaatctttttatgctatatttctacttagaatacttctatgatagacattttggtttcaccaattagcagagaatttcaaattcctcctctaaacctagtttgtttattggtttgctttccttttctttacatgttcatgtaattaagatggctttagtagtcacagcttgcacaagaaaggttgactcacctgcccgtgtgaaaacatctttcrgatttaagaagagtcccaagggagcatcccgagtttattgctctttgcaggctataagctctccttaagtgatctcaaccattttcatacttttaattaccttccctaggctaataattcctgaatctctatatttagccttagacactgccatgagcttcaatttcacatatttaattgctggctagagaaatccatttgactagctctcgagactttcaaattccaaatacccaaaatgaaactatttcttttccctataaaacatgcctcatccctcctcagctaaaagtgctacc

SEQ ID NO: 353 - DNA, 599 bp, Homo sapiens
tttcaccactgcctagctagacctctgcaaccctttaaaaaggcctacaacattttcatttcctgaaactttcagtatgttaacatactgaaaaaaaatgctaagttagttatcactgtatatatattaaatactaaactgaatgaactaatggtttcaatgcaagtaaaatggtgctattcatgagaaaattatagggcttataaaaaattcagactgcgttacgggaaatatgactaatattgaaatacaagtttaaaattgtttgataatatgttctttcaaaactgtcagtgtttrtctgagattgaatgcatactttcacttgaagataatattagatgtctatatttgagagtctctgaatgtgagtccaataccacacggcctttctgtaacttcctctgtcctgcctctccttcactgatagaactgaagcctgatccaccatagttttctcactgcctccaaacaatgctcctctcttccaaggcttgatttactaaatcttaactatctccctgcctcacttaacagagtaaaggacaatctcaacatggtattttatgccttctgggaccagtctgtgataatttggc

SEQ ID NO: 354 - DNA, 599 bp, Homo sapiens
ataatatattgaactccagtcacgtgtatgtgcctgtgtagagtgaagctgttctcccagctacccatttatactagtgcaaactgtcagcggacaagttgatatttcctttacaatctttacaaaaacatagttgaaaaaatattaaattagtgggcagtggttagagtagatcttcctaaataaaagggaccttgtacccttttcatccatagtgttatcacccaaagattttcaaaagtagatgtgacactgaacttcaattcttggtattagttgggcgtggcggtagtagtagtyccagctcttgaaaggctgaggtaggaagaacagcctgggtcagagtgagatgctgtctctaaaaatttttaattaaagaaaaatgtgaattctgatcgttgggataattcaagtatattcccaactagtgagacaggaggatggaaaaatttggggggtttatgagcaaggagttcttgcccagtacaatatgaaccctctgacattcactgcctacagtatgaaaacttgataataatgcttgtctttaaactatgttgtgaaggattggacattatgcaatttataatacatttaaa

SEQ ID NO: 355 - DNA, 599 bp, Homo sapiens
gcgtggcggtagtagtagtcccagctcttgaaaggctgaggtaggaagaacagcctgggtcagagtgagatgctgtctctaaaaatttttaattaaagaaaaatgtgaattctgatcgttgggataattcaagtatattcccaactagtgagacaggaggatggaaaaatttggggggtttatgagcaaggagttcttgcccagtacaatatgaaccctctgacattcactgcctacagtatgaaaacttgataataatgcttgtctttaaactatgttgtgaaggattggacattatgyaatttataatacatttaaagtatccagcacaaagaagaaactgagaatctggtgacaataataactattagagacagtaggaggggtcaagaaatgggaatttcatctggtcttaaagtttaaatattttaagctgcttttttttttacttcaatagactacttgggtttggatctgtttcccccacgttttgatgtatgtcacctaagcctcatttttctcagctataaaataaacctctagcaatccatattaggtagccatagatgaaacaagaaacctgcgtgtgtgtgtggtgt

SEQ ID NO: 356 - DNA, 599 bp, Homo sapiens
gtgtacacatgaaaggctcaaagttattgtgagagttgaatggttaaacgtctgttgctagctacattcatcatggccttacattgttatgactgcatgaggtcattttattagatggaacaagcagcaaaccaatctgggtcaggacaggataagagatactaagggtttttcctttctcaagatattctaggtaaaattgcattcaggatgcaggaaggtacaaaaatacttctaggggatttcctaaatgcggttattgagaaccaattttaatttaaaaagcagcagcatttggcstagaaggaggggaacaacaggatataatgttgtgcacaaaagcacatgcacaattagtagtggtaaataagggcatgagacaaagaccattgttccaccttcccaattccatctgcttgcactcagaagcttgtatgaaaagctttacatgtgatacctgtaggcaaggaaacaaagacaatgaaacaaggatggaaaatttaagatgacttacaatttcattgccccaatatcttggtcatattcccaaacctgcccatttaatcaccagctcttttgatatttagtcacatgttttc

SEQ ID NO: 357 - DNA, 599 bp, Homo sapiens
ttatgtctaaagacacaaagtcccttcagttgagctcatcttcagccttagagcttgactggattttgaaggaaacaaatcagtttgataattctgatcatgcatttctagaattgtcttcatattcatgtgatgaagtgctttactctccattaatgagctcacttcttaatgagtctagcctccctttccctcagagctcctctaggaatcttagccatgtggttaagcagtaacatctgctacccatttttagatcctcttaatgggactttttcagcgcttaacaatagaataagrtcatttcatatgtagcctcagtcttccaaagattgtttaagggtatttttgaactatttatttgtttaaaaaaaaaagctgtagtttttcctcagatttagatatgggatgaatggggaatgctgtgaatcaaatttgggataattttatcttcttgtcagggaatagcttcatgttttatcttctcacaaaatctataatgagagagctgtttagttcaagtctggtataggtcattaattactactatcacagggaggattttttcatccaagtccaacttgttggaaaactgtttt

SEQ ID NO: 358 - DNA, 599 bp, Homo sapiens
acccatttttagatcctcttaatgggactttttcagcgcttaacaatagaataaggtcatttcatatgtagcctcagtcttccaaagattgtttaagggtatttttgaactatttatttgtttaaaaaaaaaagctgtagtttttcctcagatttagatatgggatgaatggggaatgctgtgaatcaaatttgggataattttatcttcttgtcagggaatagcttcatgttttatcttctcacaaaatctataatgagagagctgtttagttcaagtctggtataggtcattaattaytactatcacagggaggattttttcatccaagtccaacttgttggaaaactgtttttttcacaggtcactgccctgactctggcctccctggactcatacatgcctggatatggagagcctgtgctccacgtgatgtctgctactgggcttgcaatgtttcctccaagtatggtgtgcagacatctgcatcagaatcacttgtgagtgcctgttataaagtattggattgcaacccaattctactgaattacagtcttacaggtaatgcccaagagtctgcgttttacacattctccaag

SEQ ID NO: 359 - DNA, 599 bp, Homo sapiens
ctcacaaaatctataatgagagagctgtttagttcaagtctggtataggtcattaattactactatcacagggaggattttttcatccaagtccaacttgttggaaaactgtttttttcacaggtcactgccctgactctggcctccctggactcatacatgcctggatatggagagcctgtgctccacgtgatgtctgctactgggcttgcaatgtttcctccaagtatggtgtgcagacatctgcatcagaatcacttgtgagtgcctgttataaagtattggattgcaacccaattytactgaattacagtcttacaggtaatgcccaagagtctgcgttttacacattctccaagtaaatcttgtaagcatttctttggaaacaactgaggaagaagtagaataatgatattacaacccagagataatatcagttatctaaatgtggtatctacctgtccttaggaaaccaacttgctctgaaaataaattaaaagggagagggttttatatttctttctgttgatttagaaaccctctaacttaatgaaatccacttcttcatagtagatttaaccatagcagatgaatagtgg

SEQ ID NO: 360 - DNA, 599 bp, Homo sapiens
tcattaattactactatcacagggaggattttttcatccaagtccaacttgttggaaaactgtttttttcacaggtcactgccctgactctggcctccctggactcatacatgcctggatatggagagcctgtgctccacgtgatgtctgctactgggcttgcaatgtttcctccaagtatggtgtgcagacatctgcatcagaatcacttgtgagtgcctgttataaagtattggattgcaacccaattctactgaattacagtcttacaggtaatgcccaagagtctgcgttttacamattctccaagtaaatcttgtaagcatttctttggaaacaactgaggaagaagtagaataatgatattacaacccagagataatatcagttatctaaatgtggtatctacctgtccttaggaaaccaacttgctctgaaaataaattaaaagggagagggttttatatttctttctgttgatttagaaaccctctaacttaatgaaatccacttcttcatagtagatttaaccatagcagatgaatagtggttcatctgggttttaggatttttatattcccacttaaaattgacttcga

SEQ ID NO: 361 - DNA, 599 bp, Homo sapiens
gagagcctgtgctccacgtgatgtctgctactgggcttgcaatgtttcctccaagtatggtgtgcagacatctgcatcagaatcacttgtgagtgcctgttataaagtattggattgcaacccaattctactgaattacagtcttacaggtaatgcccaagagtctgcgttttacacattctccaagtaaatcttgtaagcatttctttggaaacaactgaggaagaagtagaataatgatattacaacccagagataatatcagttatctaaatgtggtatctacctgtccttaggaarccaacttgctctgaaaataaattaaaagggagagggttttatatttctttctgttgatttagaaaccctctaacttaatgaaatccacttcttcatagtagatttaaccatagcagatgaatagtggttcatctgggttttaggatttttatattcccacttaaaattgacttcgatttgactttgcagcaaaaaatctatgaaagcatttaatgtccagctatatcataaaatctgttgtgtaacagaatagccaataggaacatctgggaaccaaactttattaccagtcaagcttt

SEQ ID NO: 362 - DNA, 599 bp, Homo sapiens
tgaaagcatttaatgtccagctatatcataaaatctgttgtgtaacagaatagccaataggaacatctgggaaccaaactttattaccagtcaagctttaagcttgttgccattggcatcaaggaaccagttttatgctgctccaggagactacaagaaaaatgttaagtagaagggatgccttagtggtgacatatacatctgtgcatgttttatagctctcagtacaattacttgattacttgtggggagtttcaccacctttgcttaacaatcatttttcttgtgtggtcattttcrtgttaatatgcagctggttaaatatttctttaagtaccagtaaaaaataaatacttaatgcagccagattatagcctggataaatgcagagagagagggtgggagggagtgagaaacagaatgaatgaactagaccactcacttgacactagtcttaggaatctggtctctgattccattttcaacaggatggtaactgagggaaacaaaaagtgcactggactgtaagttaattctgctctcctactatctatgtgattgtatgaatattttacatttcaaacctcttctttcctttt

SEQ ID NO: 363 - DNA, 599 bp, Homo sapiens
aatgtatttaatttcttaatactagtttgaaatatagaaaatgttatccaatttaattttaaagtaaaatatatcataagttaattaatttagtttaaatttatttgctaaaatatggataagtggttcagacttttatggatgagaacatattaaataaaacatgaatcatgtgataatattttaatttttactatctattgatttttccaaaaatcaaaatgtattagaaattcagaaacacacgaatttttattttattaatcatagcagtatctacactatcttgaaaaatagcawctatgaacataaaatagaactaattcaatgtaaaagcaattcttagtttgtataaagattcttggattctttgcaataacttcaaaaacaactccaaaatatttgggaactaccaaaaataaagaatgaagagataaacttgagcatcctctaagtggaagctagattttattcatctacacaagatataaaaaagtgagaaacagtttaaacctgacagtcaaggaactctttttgaggatgtgatattttatcctgaaacctaaaagaacagaaagaacttggtatacaaagctggg

SEQ ID NO: 364 - DNA, 599 bp, Homo sapiens
cattttgagaaaaatcacctttgctgctgtataagtaatggattgggatggggaaaggcagggtaaataaaaagaccagtaaagagactcctgcagtattccaagtaaaacatgacgatggtctgaactaaaggtgaagacaacagagataaaaaagaaatggaagaatgtgataacttttggatttaacaaggcttgatgatggtttgaattcttaaacaaattgggcaaccagaaattactctatttttctggtttgttttcctgggtgggtgatacctcttctgaaatgtggaagaytgtggatgaggaaacaattggggtgagggaaaacccactaattatgattttcaggtttaaggcattctaaagatctgtaagttcctattaatttgggcatgtcagtcatttagtgaaaaccttattcccacagtttgtcagcgaaaccttcaatcttaactattcatacttgcctgattcttatccttccacttgcagcttatatttccagtggggacttgtgtcagccctggcccttgaggcagtgtgctggaatgaggagtggcaagagcctgcttattaactagtcatgacaatat

SEQ ID NO: 365 - DNA, 599 bp, Homo sapiens
agataaaaaagaaatggaagaatgtgataacttttggatttaacaaggcttgatgatggtttgaattcttaaacaaattgggcaaccagaaattactctatttttctggtttgttttcctgggtgggtgatacctcttctgaaatgtggaagattgtggatgaggaaacaattggggtgagggaaaacccactaattatgattttcaggtttaaggcattctaaagatctgtaagttcctattaatttgggcatgtcagtcatttagtgaaaaccttattcccacagtttgtcagcgaamccttcaatcttaactattcatacttgcctgattcttatccttccacttgcagcttatatttccagtggggacttgtgtcagccctggcccttgaggcagtgtgctggaatgaggagtggcaagagcctgcttattaactagtcatgacaatatccacccacgctgtgacttcatttctgcttccttggaagggatggtgagtgggtgggtatggctctagggctgtctgcctaagaaatgtaaagctaaactattattctaaacctgaaaaatgcagcaggtagtatttttcatatttt

SEQ ID NO: 366 - DNA, 599 bp, Homo sapiens
caagattcttttctgtaatatgaatatccagttatgcagtatcatttgttgaaaagacttctccttttcaagcgagcaactacctttccactatgtattgcttaccttgaattgtactacttaaaaataccttttattcatttgcatattttatatccttatttatttttaagagaattgtaaaatgagcagttttttagttagtcagttctataaaacctgccttatcctctccagcagaagcgagtcataattgaccagttgaatctggcctatgttgtcatttccctataatggacmaaggatgtttattacacagaatacaacagcattatacactctgatgagtcagagatccaggaaaattatgatttagtgttaattcacatgatactttctttagtgctgcttactagctgtagtttatggctcaattcttaccactccctgaaagttactctgccctcaacaattctgaagaacttattcccagaaaagcaggtttctctcacctccagatatttgtacatgctcttatctttgcccgtaatgcctctatcctgatttcagagcaattccttttacaccaaaggaaacct

SEQ ID NO: 367 - DNA, 599 bp, Homo sapiens
tctttagtgctgcttactagctgtagtttatggctcaattcttaccactccctgaaagttactctgccctcaacaattctgaagaacttattcccagaaaagcaggtttctctcacctccagatatttgtacatgctcttatctttgcccgtaatgcctctatcctgatttcagagcaattccttttacaccaaaggaaacctttctcaatcccccatcagtgttaaatagtcccccctagcttcccctatggcttcttggcattagatatatttatccatattcctcgaattattcttyctacgtttctctctcctccccacacatagcctccttaacataagcctgaaaagcataaatctattctatctttctttgcacccctgtaaattatgacaaccacaacaagagtagatttgctgaatgaaagaatttgaattttctgaggtagtttaaatttcaagtctcttctaccttatcaattagtatatttatatttacaagatcgcacagattgatcacttagtctgggaattttaggcatctttttataccataaactggggaaaaacatctcccttaaaagaattttaaaaaga

SEQ ID NO: 368 - DNA, 599 bp, Homo sapiens
actccctgaaagttactctgccctcaacaattctgaagaacttattcccagaaaagcaggtttctctcacctccagatatttgtacatgctcttatctttgcccgtaatgcctctatcctgatttcagagcaattccttttacaccaaaggaaacctttctcaatcccccatcagtgttaaatagtcccccctagcttcccctatggcttcttggcattagatatatttatccatattcctcgaattattcttcctacgtttctctctcctccccacacatagcctccttaacataagcytgaaaagcataaatctattctatctttctttgcacccctgtaaattatgacaaccacaacaagagtagatttgctgaatgaaagaatttgaattttctgaggtagtttaaatttcaagtctcttctaccttatcaattagtatatttatatttacaagatcgcacagattgatcacttagtctgggaattttaggcatctttttataccataaactggggaaaaacatctcccttaaaagaattttaaaaagacatttgtaaatgtacctagtgcatattctcaagtttccttatacat

SEQ ID NO: 369 - DNA, 599 bp, Homo sapiens
aatcccccatcagtgttaaatagtcccccctagcttcccctatggcttcttggcattagatatatttatccatattcctcgaattattcttcctacgtttctctctcctccccacacatagcctccttaacataagcctgaaaagcataaatctattctatctttctttgcacccctgtaaattatgacaaccacaacaagagtagatttgctgaatgaaagaatttgaattttctgaggtagtttaaatttcaagtctcttctaccttatcaattagtatatttatatttacaagatckcacagattgatcacttagtctgggaattttaggcatctttttataccataaactggggaaaaacatctcccttaaaagaattttaaaaagacatttgtaaatgtacctagtgcatattctcaagtttccttatacatggatatcgaactaatgattgtagaaacacatttcaagtacatattctcaaagaactgtgttaaatgtagcctattcttttccaaatgttgttttctcctggtatactatttgagattctgaagataaaacatacaaataagtagatggagtcccaattttgc

SEQ ID NO: 370 - DNA, 599 bp, Homo sapiens
tcccccctagcttcccctatggcttcttggcattagatatatttatccatattcctcgaattattcttcctacgtttctctctcctccccacacatagcctccttaacataagcctgaaaagcataaatctattctatctttctttgcacccctgtaaattatgacaaccacaacaagagtagatttgctgaatgaaagaatttgaattttctgaggtagtttaaatttcaagtctcttctaccttatcaattagtatatttatatttacaagatcgcacagattgatcacttagtctgrgaattttaggcatctttttataccataaactggggaaaaacatctcccttaaaagaattttaaaaagacatttgtaaatgtacctagtgcatattctcaagtttccttatacatggatatcgaactaatgattgtagaaacacatttcaagtacatattctcaaagaactgtgttaaatgtagcctattcttttccaaatgttgttttctcctggtatactatttgagattctgaagataaaacatacaaataagtagatggagtcccaattttgctatttatcagctgtgtgaaagta

SEQ ID NO: 371 - DNA, 599 bp, Homo sapiens
gcctattcttttccaaatgttgttttctcctggtatactatttgagattctgaagataaaacatacaaataagtagatggagtcccaattttgctatttatcagctgtgtgaaagtatcagtgaggacagttggggtgatgttgcaataacagagaagctgaaaaatcacagtggtctcactcccacaacaagactaatcataagtcagctgcaaccctatttcttgttgccatcactctgggaatcaggctagtgaagaagcctgtatttaaaatattgccagtcatcaagacaaaggraaaagagacatggagaaccacactgttaactcttgaagcttcttcctgggaatgctgcatgtcacttctattcacatttctgatagccacctctgagttcatcagggcaggatacataatcctcatgcagagaaggaagttgaaatagtgtgtttcacaaaagcaatttagtctaccaagtaaataatcttggggaaaacatgtattctgtgttagtttcctcatcgataaaatgacaataatgcaatgatgatagtaactacttcacagggttatgtggattagcagagataatgaat

SEQ ID NO: 372 - DNA, 599 bp, Homo sapiens
aggaaaaagagacatggagaaccacactgttaactcttgaagcttcttcctgggaatgctgcatgtcacttctattcacatttctgatagccacctctgagttcatcagggcaggatacataatcctcatgcagagaaggaagttgaaatagtgtgtttcacaaaagcaatttagtctaccaagtaaataatcttggggaaaacatgtattctgtgttagtttcctcatcgataaaatgacaataatgcaatgatgatagtaactacttcacagggttatgtggattagcagagataatraattcaaaatacttagtatagtacctgacatgtaattattactcagtaagtgttagtttaaaaaataaaaggggaggaatctcagtttctgtatctgtcaaataagtaatagactaattgatacccataatatctgggacttcagccaggtggttagaacaactagagattactagaatggctcaattgtggacataggtcttgaatctctatttttctccatgtggagtcttttggggttgaaaatatccaaaacgactccctcactcacaggttcattccctgagctgggatagctg

SEQ ID NO: 373 - DNA, 599 bp, Homo sapiens
tcttgaatctctatttttctccatgtggagtcttttggggttgaaaatatccaaaacgactccctcactcacaggttcattccctgagctgggatagctgtaacttctgggggctggccaacatggctctctagccacacatggtgggcccaaggtagccttacttcttatatagcagctggctacgctcagaacagacatttcaagagagagtattctaaaaggcccagaaagatgttataaggcttcttaagacatagcctgagttccagaacctcagtctcactgcataccattagkcaagcatatcactaaggccattagggttcttatctctttactaaagttgagtgaagaacttgagtgggtgttgttaggggaatacatattgtagtaactgtgtggtggtttatttattttttgtttatttattttgtattccttgtgcttacttttcatctatatatcttctttagtgaagtgtccattcagatttctggcccattgttgaactgagctgtttgttttcttattttgagttttgagatgttttaaaatgtatagtttaaatagaagtcctttgtcaagtatatgattta

SEQ ID NO: 374 - DNA, 599 bp, Homo sapiens
catggtttggcattatatctaagaaaattacttaactcgaggtcacaaagattttctgctatgttttcttctaaacgttttgaagttacattttaaagttagctctctaaaccattttgagttaatttttgcataaagtgtgagacagaggttatgcttccttttttttttttttttgtataggatgtccaattgttccatactatttgttgacaagactatccatttttccattgaattgccttcatgcctttgtcaaaaatcaattgcccatattgggtatatttttggactctaatytattcattgatttatatgtctatctctttcctaatgtcacactatttttattactgtagctttatagtaagttttaaaataagatagtttggattctctaactttgtttttcttttacaaaatggttttggccattctaattctttttgcctttccttataaattttagaatcagttatttaaatatgcaaaaaattattttggtattttgatttgaattgcaactaatctataaatctacccgggaagaattggtagcttaactgtgttgagtattccaatccatgagacctgtatat

SEQ ID NO: 375 - DNA, 599 bp, Homo sapiens
taaatttctgattttttcttgccatattgtactgtccaagacttctggttgaataaacatgaagagagtggatatccttccttgttcacagtcttaggagtaaagagtttggcctttcatcattaagtataatgttgattaaaggtagatgctatttatcagtttaagaaagtcctcttcattttcctggtttcttcagagattttaccataaatacataatacattttattaagtactatttctgcatgaatagttataatcatctgatttacctttttaatccattatgatgacaacytttatgaatagttttttaaggcttatttgtttaagtagagacaaggtctcgctatgttccccaggctggtgtcgaactcctgggctcaagggaggctccagcctcagcctcccaaagtgctgggattgcaggcatgagccaccatgctcagcctgttttttaaatgctgaactatcttgcatctcaagataaatttcacttggttgttatgtattgttctttttatataagtctagatttgatttgctaatattttgttgaggatttttgtgtctatgtccatgagggaaactggtctc

SEQ ID NO: 376 - DNA, 599 bp, Homo sapiens
tagatttctttccctgtactgcctttttacataaaggtaattctgccctcataaaataaatttggaagtactctttctttaatttcctggaagatattatatagaattggtgtgatttcttcttttaaaatattcaatacaattcaccagtgaaatcatttgggattagagatctctgttttggaagatgttaaactattaattcaacttctataataggcatagatctataaagtgagagtgagtttggtatcttgtggctttcaagaaaatgaccaaatttatgttgttgaatttatrtacttagagttcttcttggttttcctttatcagccttttaatgtctgtagtgatacatcatctttcactgctgatagtaataattttgtcttttctctttttttacctggtcattccagctgtatgtttatcaactttattagtctcttctagaaaaaaagcttttgatttcattgatttttgtctctattttgttattttttctatttaattgattttggaccttatgtttttttctgatttttttgggttagtgttgcttttcatctgctagtttcttaagtagatacttaccttgt

SEQ ID NO: 377 - DNA, 599 bp, Homo sapiens
tgtatgtttatcaactttattagtctcttctagaaaaaaagcttttgatttcattgatttttgtctctattttgttattttttctatttaattgattttggaccttatgtttttttctgatttttttgggttagtgttgcttttcatctgctagtttcttaagtagatacttaccttgttgatttaagacttttcttcttttctaatgtaatcatttaatgctataaatgtccctctgaatgctgttttagctccattacatacaattttatatgttatatttacattttagttcaggtyaaaatatttcctaattttctttgaggctgtctcttagatctcatggattatttagaagaatattttaacttacacctatttgagaattttgcagatagctttttgttattgatttccagtttaattatattagtgtcagagactatatttcatatgaattcaatttttaaacatttgttaggttttattttttgacccagacctggcctatcttggttagcgtttcatggtcactcaacaggaaattaaaatctacagttgtttggcagagcgtcctattaatgtcattacatccaact

SEQ ID NO: 378 - DNA, 599 bp, Homo sapiens
taatactacttagtaaactcccatatatttatatatatatatatatatatatatatatatatatatatatatatatatatatatacaccagctaatacatctaccctcaaggcctcttagggatgtattgggtgcttccctagtcagctcccttttccgttcctcatgatgaaactcttttcctttcctcatgatgaaactctccaacatctccaatagaacagactggagacttctagtgacttttgcctgacaatgccttattctcactttccgaaatccagttcatcagaggaaatrtaagtggacatcccattggctaatctctcacctttttcatagccatgaactatgttatttttcatgcacactatacctaaaaacccatcatctgaatatcagaactggtcctaaattggatgcttcagtgggagatttgtcttcagttgcctgctatactcacattccacttgcagcacctttttctgtcctctcagcactttgctcaaactatttaactagccagaatccctaggctttccagttctcttctataagacaagaaattaatttaggacaaatgaaagcatactcaactg

SEQ ID NO: 379 - DNA, 599 bp, Homo sapiens
taaacacaggcatgcacacaccacacgcacacacgcaaacacacacacacacagagttttgttttgttttttttgttttgcattgtattctttggggcaagagggtaaattgtgatgcaagctctcaaggcactacacgtatataagtggaagtaatatataaacatgtatatattatgaacatatatgcacacacatatataacatatatacatatatatgaaatcaaactttctgtttaacccataatgatataccaataaaacaggaattttgggaaatgctggtgtcatattggaraagattttagagtcaaaagaactgggtgtactctaggcaccttaagcaagttactttatctctttaggatgtatagtcctcatctgctaaacaggattacaagagtttgacaactagccgagataaggtatgtaaagtgtctggcacctaggaaaatatcaaaaggtagtaataattatataaggaacagcatcatatctgaaatgaagtctataaagatttcatgcatgcccacctgtatagacatcaaggctaatagcatgtttgattttatcaacaccgtttgcgagctagttcag

SEQ ID NO: 380 - DNA, 599 bp, Homo sapiens
tcatctgcttacttttccacatagaagttattctttttttcttctcatatcctctatttgaccgctcaaatactctccatcttgcctgaaacaaaaaaagatgtgaatataacataatagtagaaaactttcatgttcgatccccctttgttctacttccctgtgttgtggcagtgaaattggacagaatctctatgaggcaatgtgtcacaaatggcaggaatacaatacttaatacaccaccatctctctctgacttatagacacaaacacacacacacacacacaaagaaacccatrgtatagtagatagtgtttcatgtgactgataccaaacaaaacacaaacaatgactggattattgcaacaacagagtccatgccatggtttctgggaacctttggcagctatctgcaattttaagtgcttgttttgctaactttatgttagcaggactgtgagcttcaagatgtgtgccttgtccatttaaagaagttccactcattctgtgtgcttctcaccacatgaaaataaagccagagcaaacaaataatgctcagggctgagtcacttcacaaacctcactcgaaaagagtcta

SEQ ID NO: 381 - DNA, 599 bp, Homo sapiens
cgtccctcctaacaatcctgtgatatagatagtattagctttctaacatagaaaatgaaaatttgatccagcagttaagcaacatgtgccaaaccctcaggagaacctagattgtagagcaggtactctaaaacccaacttaaatcttcctaatttacacctttgtatgccttttactgcattcatgagcatggttcagaaaatgtttaggaaacacctttgcctaagaaatatttaatgagggcaaacagtacaactaagaatagtgttattgctggcagcttccactaaatctatgcrtacagtataagaaggtaaaaaaatcagtgaacatttttgaaggatgatagttgtatttaaaaagcaaatcgtaatcttctctcacaaacacctccaaaggtgcttaatgaaaaacgagaaggtgtgaatgttatgatctcagctcacactgaggattcttgtaactataatgaaaaatccttgatagattatttaatctatttaagtattggttttctaatacaaaatcagaattgcacaacttgggtgactggtaaaaagaatgagtaactcggtggtgatacacttaagttcacaat

SEQ ID NO: 382 - DNA, 599 bp, Homo sapiens
cagttaagcaacatgtgccaaaccctcaggagaacctagattgtagagcaggtactctaaaacccaacttaaatcttcctaatttacacctttgtatgccttttactgcattcatgagcatggttcagaaaatgtttaggaaacacctttgcctaagaaatatttaatgagggcaaacagtacaactaagaatagtgttattgctggcagcttccactaaatctatgcgtacagtataagaaggtaaaaaaatcagtgaacatttttgaaggatgatagttgtatttaaaaagcaaatcrtaatcttctctcacaaacacctccaaaggtgcttaatgaaaaacgagaaggtgtgaatgttatgatctcagctcacactgaggattcttgtaactataatgaaaaatccttgatagattatttaatctatttaagtattggttttctaatacaaaatcagaattgcacaacttgggtgactggtaaaaagaatgagtaactcggtggtgatacacttaagttcacaatattattgcctgtaaatttcctgtgaatacacaatcaactgacaatcctttattgcccatatcagttctaga

SEQ ID NO: 383 - DNA, 599 bp, Homo sapiens
agagcaggtactctaaaacccaacttaaatcttcctaatttacacctttgtatgccttttactgcattcatgagcatggttcagaaaatgtttaggaaacacctttgcctaagaaatatttaatgagggcaaacagtacaactaagaatagtgttattgctggcagcttccactaaatctatgcgtacagtataagaaggtaaaaaaatcagtgaacatttttgaaggatgatagttgtatttaaaaagcaaatcgtaatcttctctcacaaacacctccaaaggtgcttaatgaaaaaygagaaggtgtgaatgttatgatctcagctcacactgaggattcttgtaactataatgaaaaatccttgatagattatttaatctatttaagtattggttttctaatacaaaatcagaattgcacaacttgggtgactggtaaaaagaatgagtaactcggtggtgatacacttaagttcacaatattattgcctgtaaatttcctgtgaatacacaatcaactgacaatcctttattgcccatatcagttctagatatggtaagagatcaaaacaaacatatgacacacatcatcctgg

SEQ ID NO: 384 - DNA, 599 bp, Homo sapiens
atgagggcaaacagtacaactaagaatagtgttattgctggcagcttccactaaatctatgcgtacagtataagaaggtaaaaaaatcagtgaacatttttgaaggatgatagttgtatttaaaaagcaaatcgtaatcttctctcacaaacacctccaaaggtgcttaatgaaaaacgagaaggtgtgaatgttatgatctcagctcacactgaggattcttgtaactataatgaaaaatccttgatagattatttaatctatttaagtattggttttctaatacaaaatcagaattgyacaacttgggtgactggtaaaaagaatgagtaactcggtggtgatacacttaagttcacaatattattgcctgtaaatttcctgtgaatacacaatcaactgacaatcctttattgcccatatcagttctagatatggtaagagatcaaaacaaacatatgacacacatcatcctggaggagcctacaggaatttggtgagacagaatttacagacataaagaattaaaatgcactagaagataataaagaatcaaattttatgaaacatcttgctctatctatccactattgttatcc

SEQ ID NO: 385 - DNA, 599 bp, Homo sapiens
cagggtaaactttagttgttttttggggaaaaatgtggataacaatagtggatagatagagcaagatgtttcataaaatttgattctttattatcttctagtgcattttaattctttatgtctgtaaattctgtctcaccaaattcctgtaggctcctccaggatgatgtgtgtcatatgtttgttttgatctcttaccatatctagaactgatatgggcaataaaggattgtcagttgattgtgtattcacaggaaatttacaggcaataatattgtgaacttaagtgtatcaccaccragttactcattctttttaccagtcacccaagttgtgcaattctgattttgtattagaaaaccaatacttaaatagattaaataatctatcaaggatttttcattatagttacaagaatcctcagtgtgagctgagatcataacattcacaccttctcgtttttcattaagcacctttggaggtgtttgtgagagaagattacgatttgctttttaaatacaactatcatccttcaaaaatgttcactgatttttttaccttcttatactgtacgcatagatttagtggaagctgccagc

SEQ ID NO: 386 - DNA, 599 bp, Homo sapiens
acaaaatcagaattgcacaacttgggtgactggtaaaaagaatgagtaactcggtggtgatacacttaagttcacaatattattgcctgtaaatttcctgtgaatacacaatcaactgacaatcctttattgcccatatcagttctagatatggtaagagatcaaaacaaacatatgacacacatcatcctggaggagcctacaggaatttggtgagacagaatttacagacataaagaattaaaatgcactagaagataataaagaatcaaattttatgaaacatcttgctctatctawccactattgttatccacatttttccccaaaaaacaactaaagtttaccctgtgaagcctttttttgaattttctagcttatagattcctctcttaggcactgtctatttaaatcacatctttatcaattagtatatatctttgcctttttaattaaaaaaaaatatacaacctacttctccgattagtttataagtctcttgctcaaggtccatatattattccttctcatatcttcaaagccagatgctttgttttgcacaatgaaggtactactcattttttccagattatcataaa

SEQ ID NO: 387 - DNA, 599 bp, Homo sapiens
cccatatcagttctagatatggtaagagatcaaaacaaacatatgacacacatcatcctggaggagcctacaggaatttggtgagacagaatttacagacataaagaattaaaatgcactagaagataataaagaatcaaattttatgaaacatcttgctctatctatccactattgttatccacatttttccccaaaaaacaactaaagtttaccctgtgaagcctttttttgaattttctagcttatagattcctctcttaggcactgtctatttaaatcacatctttatcaattagyatatatctttgcctttttaattaaaaaaaaatatacaacctacttctccgattagtttataagtctcttgctcaaggtccatatattattccttctcatatcttcaaagccagatgctttgttttgcacaatgaaggtactactcattttttccagattatcataaaaatgatgacaattgttaatgaagtatgaattcacagaacaaagcagaacaaagcaaccacaaagacaacagctggtaccactgaattcactttactgtgagatccagcagcgtgataattatgtcattttca

SEQ ID NO: 388 - DNA, 599 bp, Homo sapiens
gaggagcctacaggaatttggtgagacagaatttacagacataaagaattaaaatgcactagaagataataaagaatcaaattttatgaaacatcttgctctatctatccactattgttatccacatttttccccaaaaaacaactaaagtttaccctgtgaagcctttttttgaattttctagcttatagattcctctcttaggcactgtctatttaaatcacatctttatcaattagtatatatctttgcctttttaattaaaaaaaaatatacaacctacttctccgattagtttayaagtctcttgctcaaggtccatatattattccttctcatatcttcaaagccagatgctttgttttgcacaatgaaggtactactcattttttccagattatcataaaaatgatgacaattgttaatgaagtatgaattcacagaacaaagcagaacaaagcaaccacaaagacaacagctggtaccactgaattcactttactgtgagatccagcagcgtgataattatgtcattttcatgttttatctcttgtttttgtcctaacatgatgcctgacacatattagatatgtcaattt

SEQ ID NO: 389 - DNA, 599 bp, Homo sapiens
gcaaccacaaagacaacagctggtaccactgaattcactttactgtgagatccagcagcgtgataattatgtcattttcatgttttatctcttgtttttgtcctaacatgatgcctgacacatattagatatgtcaatttttgacaaaggaagaagagtagaaagggagagagaattgaaggaagggagaaatgaatctgggtattaacaaatggactggaatataagacaaggtgcgaattctaggcaagaggaacattatcaacaaaggacaggtggttgatattgacagtgtgtgartatgtatgtgtttgtgatgttatgtggaggccattccaactggaacaaaaaatttattttattatggacattatgttgaacagatgagacatgctttatgctgtggaagccatactgtgaaaattagaataaataattcaaaattggagctagaggcaacaaagccattacagattcttaagatgatgaaaagtatttgtatttgttttaaattacttagaaagagcatcttgcaagttaggtgagaaaactacattcagatcaaaatgtgatcatggagtgataaactagaactacag

SEQ ID NO: 390 - DNA, 599 bp, Homo sapiens
ttcagatcaaaatgtgatcatggagtgataaactagaactacagcaggattgccaaaatagaaaaaaaattactgaaagaaaatcagtggggtttgatgattaactgaatgtaatagtaaaagagatagagacatcctaaatattagcaaatctctagatggtgggagaggtggaactcacaaattgtggggcccatacattaaaataaggaagtatgaaaaatagttggtttgaggaaagttataaatttaattttggatatggtgattttatagagtggtgcataattcaaagggatrtactgtggattttaaaaataaagggatagacagaatgaaaagccagggaattgggagtcacatgaggtagtagaaaggaccccatattagagacaaaattacaaagccacaagttctgttcagagcaatggagaaggtgtttagggcttgaattattattttctttaagtcacttaacatctctgagtatttatgtttcttttttttttaatttttttttttttttttttttttttttgagacagaatcttgctctgttgcccaggctggagtacagtggcgcaatctccgctcactgc

SEQ ID NO: 391 - DNA, 599 bp, Homo sapiens
aaacaaagaaattattttctgaaaaaacaaatcctaggactggttgagttttcttctgtcaagcaaacacttttattttcttaattataaaataaaaacaagacacccctactcacttatcaaatttgttttacaggcttcagcaaagggacctgaacaggtgcaaaaacagagaccctgagtaggtagaagagagaggcagtggtgctggtgacaaggctatatggccctctgaaagcaccgatctacaaagaactagaccacatagagacttgatcactggggattttgactagaaasgggagaaagcatttaataattattaagcacatgaactctggattcagacagaggggtgtttatcctcagataaaccacaacttctagctgtgtagcaatgggatatttactggtgagctaagccactaattataatatggagacataagacaacctacctcctggtggtgttgtgaagatatgagatacagagtaagtactcaatatgcactagttgacattattataaggaaatagaattaaatctgcattgaccagcatgatgaggacatcactcttttctacttgaagttgttaaa

SEQ ID NO: 392 - DNA, 599 bp, Homo sapiens
ttatcctcagataaaccacaacttctagctgtgtagcaatgggatatttactggtgagctaagccactaattataatatggagacataagacaacctacctcctggtggtgttgtgaagatatgagatacagagtaagtactcaatatgcactagttgacattattataaggaaatagaattaaatctgcattgaccagcatgatgaggacatcactcttttctacttgaagttgttaaatataagaataaagaaagggataagagagaaaagggcaagaaaggaagaaaggaagagagrgaatgcagcaaggatgggtaaataaagaaggggaaatgggataaaaatgagaaagaacgaagaggcaaataatggaaaagtttgaaaagagcaatggtaaaagaaaacagggaaataatgatttaaattaatacttatagtttcagatttctatatatttttccttggccattataatagaagcaggacaaaataaaatataactgggaatgaaattggatacaaattttacaggaaaattcatgggaagcagaagaaaaagggagaaaagagccttggtgttggacctatgagaattg

SEQ ID NO: 393 - DNA, 599 bp, Homo sapiens
actgggaatgaaattggatacaaattttacaggaaaattcatgggaagcagaagaaaaagggagaaaagagccttggtgttggacctatgagaattgattgcaagtgtaaaatatgacttcatctagttcttttcagggccttcttttttcatatctctgtcactatttaaaaatatttcttaaataatgtacaatgcaagccttcttagtcaaatatgccttggcctttatacactactttaaacaaagtacacttacataattaggaacggtgctataatacaaagcaaaggtattayattacaccaggaaacttcagttctataaaacagaggttatattttagcaaactagcaaatctttttttaatccctattcatacacaattcagaatgatgtaatgccaggttatacatcaactaaactattcttttaaggcatctctgacccaaatgcctgtggaatctgtctggagttgatagtatgctgaagaaatgaataaactctgtgcttgatcttctatagattaccacagttctcatacaagaaccaaaaataactcaatataagatactcacattatgcttagaaaatgaat

SEQ ID NO: 394 - DNA, 599 bp, Homo sapiens
tgatcccactacttagcacaggaggttcgacctcttccatttcccacctggtccagtgtaccgccctttgggcaacctggtggttttctgctcctagaaggtcacattattgatgccaaacttagtgcagacaccagatcagcatagcatactacagctcagaactcctggactcaaacaatcctcctacctcagcctccccagtagtttggactataggcagacacctgcctttgcaaactttcaatctgtaagatgtgtacatttaaggttggccacagacaaaattgtatagtacckgcagttgtattccatggctcattacatctcaagctcctctcctactcacacacccagacatacatacacaagaactctatctccttctacacaggactcccaatcaagtccctactctatagttttggttttggcccttcccatgatactatcttctatctcattctccctccagccagtgatttgctaactaagatggcatgacacaaaccaggcaaagagttcctgaatattcatgaattgaatttctttctagttaccatatttgcaattgaaggggaaagttaacaaaaacaaag

SEQ ID NO: 395 - DNA, 599 bp, Homo sapiens
caagtagctgggactacaggtgtgtgccaccacgcccagctaatttttgtatttttagtagagacggggtttcaccatgttggtcaggatggtctcgatctcttgactttgtgatcagcctgcctcagcctcttaaagtgctgggattacaggtgtcagtcaccgcacctggtccttggccacctttatacctctgccaaataaaaagaaaagtatgaaacaaaaagaagattccagttatctcatattattcacagcctcatgtctaaactacacctccaagaccccacatcactctayagtgttatgatttgaagtctgtctcccacaaaagatatcctgaaatcctgaccctatggtacctcagaatgtaaccttatatggggaatggggttgttgcagttataatttgctaaattaattcaatatggtgggtgtgcgtgtaagaagacagaaatttagacacagacacagaggtaaaatagccatgtaaagacagaggcagcgattggagatatgcgagcacaagccaaggaatgctgggcctcccagaagctggaagaggcaaggaggaatccttccctagaggctttggaggg

SEQ ID NO: 396 - DNA, 599 bp, Homo sapiens
tgttcctatttaattagccactagtttttctttctttttaatgataaataagaggctccctttccttctccgtgtacacaacatagctagggaagaaactcagtctcttaaagtcctgcatagtttaacattcattcttagattctttatgcattttgaatagaaaaaaaaggcaaatccagctcatacatatggtggtcttaaaaggaagcatataatgaagtataaaaaattacatcacaaataaaaacaatacaataaccatttttacaattttactctgcaatgtattattagacytggagaaacactgcgagtttaggtggttgttggcatggcaaatgctactaatcaaagaaacagaggtagttggagatggaccagaaaagtaggagcaatgagggagtgctactaagagtgatatgactcaagaatttttccctctgtctttcttagttctccctatgttccctctcccagttgttgctgaattcactgctacccccaacccccatcctggactcattactgcaaagtcttctcaactgattccccaactcaagatgtaattagcttctcaggctaagagcaattggtaa

SEQ ID NO: 397 - DNA, 599 bp, Homo sapiens
tcttctggcctcactccctcacccctaaccccttagctaagactacaagtgcatgccaccatgcttggctaatttttaaattttttgaagaggtggggtctcgctatattgcccaggctgactcagtttcttgagaatcttgaaaacaaacaaacaaaacacatgctcaagaaccaaagtaaaaaaaaaaagttttcaaatagtttgagtaagaaatgtcctcagattaaaccacataaagcagttatggtagcattaacaacaatctattatagatccataaacattcaatgattttamtatattctgaaaactagctaaagcatcattcaaaatgtcttcattttaaactaaaatgtcaataggcatttaaccgaaaaaaaatttaaagcttgacatactaagctttccttcaaaagtttacatttttttatatttcactggaacaaagttactttgcatatacatcacacttttactctcagaggagtttctcctgcagacttctttaaaaaactatatacatatatacatacatatacgtatatattaaattatcctcagcctcaaatactgattttgcagtctgtgattcatag

SEQ ID NO: 398 - DNA, 599 bp, Homo sapiens
ctagactctcgggtactttgcgccccagcttcagctcgatgtctctgaggtctggcagcgggctgtcttccatgacttttccggaacggcgataatagtaataaaacgcagcagatgaaaggcagccacggcttcgaaggctgctgcgaaatcagcagacctctccgggggccagggtctctatcctccgccgctccggtgtaccgtcctttccataaccgcctatgcggtatcagccctgaaatccgcacaggacgcggggggtcgcagctgcagttcccagtccgctgcgactcctcygacactaagatgatcaagatctcctcattctctctctttttggccacctcactgcaaagttgagttcactgatattgcagacagagggggaataaagagggggaagtagtgaaagaaaagagagggaaattaaatctgttttagctttaccaagggaaaattttcacattataaaaccgtccgttcacatccctgcgccgctcactgactgcccatctggtactacgcgacacaaaataatgatgcagccagaaacgatgtcctgggtcgtttcattttccagccatatgcctccaagc

SEQ ID NO: 399 - DNA, 599 bp, Homo sapiens
gcggactttgctcggtcacggtttgcaaagacagcacagtcggctggctccactctgtgtactttcggacaacgcgactcactgcgccgggctctatttagctgccagcccgtagcctcttaggcaccagcccgttgagagtttgcagttattgcacccatctaaggcctgctgtgcgctgttaacacttcgggtaggttcaaaaggtggctcagtccaatgcacttactgcaaaaatacttcctggtttgccgggaggtggcgtggcagggtccttgcagcctttcctcctagctcacmcttccaccagtaccgctccgccctcctgccctcccttcttggtgcagcagccgcagctgttccaaggccactgtcgagcagattctccacagctggttctgaaaacaatggcctactcgtccttgatgtacttatgaatagcttgtttcactattttgatcccagggagcaggtgatccccaggcccagggatctgctaaggaccatctcccacccttctccctcaagtctcaatctgcagttgttttccctgctgtttgcaaacgtttgcctgagcataggacaatgagataagtaca

SEQ ID NO: 400 - DNA, 599 bp, Homo sapiens
aacaagaaggaaattgtgggtgatgagatcaattgtcaaggtctggaggccaagttccatttaaataacataaaagcaaagaaaaaaaagaaaaaagaaaaaaagaaagagaagaaaactttaagtgaaaatttaagaatgaattatttcggtaaggcttatctgtaagttaatgtacctgctaatatataatgattttcatttttgttataaattgaacattttttgttcaagtacactgtatttagccatttttgatatttttaatactcagagaatttataaacattcctaaaatcyttttatgaaccttatcttttagaatgatagaagtctcttgattattcccttgtggtaccatattaaaaatgcaaaggaaattttctaactcctcaaaccacacaccagtggcattccaaagcctacagaatagctagtgaatatgaagttaaccagtccaaatgacctttccttccacttattcttacggtaaattatatgctgtaccaatattaatgtacttgcagcttcctaaaattcaacaaatatttattgaacatctacaatgtacaaggcaaattttttttttctttttttga

SEQ ID NO: 401 - DNA, 599 bp, Homo sapiens
aaaatatataatttaaaatatgtataattctaatgaaatatgaagaagtgttgttatttcaatgggaaagaattctttatttactaaatcatgcaggcacatctgatcatctatctcaaggacctcgcaatgcaccaattttaatttaaaaaattgtagttggattaaatgaataaagatacaaaacaagaaaacaaaactttgccagttttcagagaaattttttggatcaaaaagaggagtgaagaagctataaaataacttttaaatatgtatttgattatgtacaagcaaaaaacrgcaaatttagcataatcaatatcctagaaccaaagtatatattgaggggaaatctgtatgtcaatgattataaagccaaagagaaatgtgaggaagagaatgtccatattcacattggttacctggggagaaagtaagaagagacaggaagaagggagaggagaaggtagacaaaaggaaaaacaaacaaacaaacaaaaccacaaaaaccaaaagagataatggaaagaagaaaacaaaacaaataatggaaaaataaaaaatatatatgcactagcataatgtctacttatattaat

SEQ ID NO: 402 - DNA, 599 bp, Homo sapiens
ccaagcctcctgagtagctgggactacaggcgcccgccaccacacccggctaattttttgtatttttagtagagacggggtttcaccatgttagccaggatggtctccatctcctgacctcgtgatctgcccgtcttggcctcccaaagtgctgggattacaggcgtgagccacggcgcccggcccaaatcatttttaaaatcttgcatcaagtgtgcctatcccacccttatttattaggccacatgcccctatcataggatctcccaggacttgcttcctttactttttagcacacarcaaagacaaaattttgcatttgttaagtgataatttgcatccttccaaccagaccatgaccccaggagagtagtgaatatttgctttttcttgtactataacaccactgttcttttcatgtcagcatatctctctgctaagtttagtgccaagtatatagtatgtgttcaggggcatcttttgagtggaggaatagatgaaacttcaaatccagtgattggtcacattattaataatcaatcaacaagagtgattttcattactgccattatcattctgctgctattttatgtttaggg

SEQ ID NO: 403 - DNA, 599 bp, Homo sapiens
taagctaagtcatcatctttcctgtaatgggagaaatagtagaagtccatactaagaatgtggaccacagtagaatttagtgatagactgttggaattgctaaagatcagtgggtgctgacagtcagagaattaaaactataacactgttagaggagtaaaaacaagccaaattatggccatgatggtcctcattcaattacactagtaggtagtccacttaaaaaactccatataatctggtctacatgtcctagacacagaaccggaggaaccatcgattccagcttcatgcctatcyggtttttgatgaattaatgatccattatccctgtgagaccattcattacatattattctatgttaaaaataatgtgtgacatacatttatgcaatatatactatgtgccaagcatgatccttagggctgtattaatcattatctcatttaattctcaaatatccttccatgtaatcacaatgaatcttatttggcaggaaaaagaaaaaactggcttagtgagattagtgctttctaatggtggcagaattagggctcaaatccaggatattctgtttaccaaggctatttcctttcca

SEQ ID NO: 404 - DNA, 599 bp, Homo sapiens
gtaaactaaaaaaagcatataatatatatacatacatatttcaattataatagaacatacatttgttgtgagaatgagtgccttaaaataaatattatctgaatattaagcatattttcccaacagtaccctggcaccaggagcctcgttctgtgcaagcactcctactcaccagctctgtgaccttagacaagttgtttaaatttcctagacctgagtttcttttctaaaaatgaggaggttaaaaaagaacaaatactagcaggaactaacatttttgaacccttattgtgtgccaartattacgctaaattaatgatcacaactgtcctatgagccaacaatattgataatcatcccttttttgaaatgtggtaatttaggcatagggaacttgcctcatttccacagcaaatagtgatggaccagcaaataagataacacctggtatttaaactcaagtgtatctgaattcaatgtctgcactctttcttgatgactactcagagctcccactgttagaaatctcagatctgtcccttccagatctgaaacttaatgaatattgttaattactgatggagcataaatatgtgaaa

SEQ ID NO: 405 - DNA, 599 bp, Homo sapiens
ttttcattaacaaaaatggcactctaactttattagggaaagtcaaggattgaaatcctaacccaaatctagtgaaggtcaaatatatgacagttgaaaagagggtatataaccaagcaatatgaagacagtttctgaagaaatcattaaaaatacaaataattatatttttattgttcttgaggaaaggaatgcagaaatatcttttcattctatttttattctatctacttttcattgcagctatgaatgagactactgactttcaggtagtattaataatatattaataacagcacraaaatccaccttgatatcactttatagaataaaggaactttgacaatttctgaaaataaacacttcataaataagttatatattcattgttataattggaactacaacaatctggcagagcatttaatttagttttcaaactaatgcagcaattaaatgtaatctgcctatttcctagggtccatgttattttttttggttttaaactcaggcccatggcatatctcaatgtactacaacttgtttctaataacgaagtttctagttggagaagtaaaggcacataagcatgtccaatt

SEQ ID NO: 406 - DNA, 599 bp, Homo sapiens
aattcgatgatttataaaaagtattttgtatcttcttcagtattctatagtgtttaatatacacatttttttttttctagaagaagagctgcataacaagcgagtctgcccatttgggtcacaacctctgtaggtcaaaacaaattttttccaaacagccatggtcaagaactaatgatttttaaaacaccataaaaaacaaagggctacttattattaactattataaaacatcagtcagatttttctttcctcactattcttggagcacaaagaagcaataaattttaaaacaaacaygttgaattatttaaaattatacttctcaaatatgttctatacaaggataaaattaatttattcttatctccattttggatttgaccatgaattcttctgaaaaaatagctgtaagaccaaactttttggtaattctctataacaacacacctaggttagaactggctactaccttcaatcctatgtgtatttatgtgtaccttgctttaactattagaaacaataggcacctagaacaatcgcgaaatgacaaaaatatgaggtgttcaatgaagatatagagacttgaaaacctctag

SEQ ID NO: 407 - DNA, 599 bp, Homo sapiens
ttcgaagagggtttgcgagacaagaaacaaaataaatacgtaaaatattgtgtgctacatggtgctgagtgtttaaaagcaaaaaaaaaaaaacaaaatgcagtgaagcagtatagaaagtgccagagatataggaaattttgagtaatgtggccaaagaagcataaactgaaaaaataacatttgagtaaatagctaaaagaattaaaggaatgagtcatgcagatctctagagaaacaacattccaggtaaaggataaagcaaatacaaagcccctaagacagacttaatatttagtrtgctgaggagtagcaaacagcctcatgtggctgtagaaaagttaaaatgcagagaaaggaagacatggagaacaagaggtaatgtggtgatgttgaacctgtttggactttaaaagactttggccttaacatttcatgagaacgtttttctgagaggattccaacagcggaggaacacattctacctaaaattttaacaagaataagggcacgaaggcagaaaatgaagagaaaattaggaaacttgcactaacttaggtgagagagatgatagtcgttggattggagatacggtaagt

SEQ ID NO: 408 - DNA, 599 bp, Homo sapiens
ctagagaaacaacattccaggtaaaggataaagcaaatacaaagcccctaagacagacttaatatttagtgtgctgaggagtagcaaacagcctcatgtggctgtagaaaagttaaaatgcagagaaaggaagacatggagaacaagaggtaatgtggtgatgttgaacctgtttggactttaaaagactttggccttaacatttcatgagaacgtttttctgagaggattccaacagcggaggaacacattctacctaaaattttaacaagaataagggcacgaaggcagaaaatgaasagaaaattaggaaacttgcactaacttaggtgagagagatgatagtcgttggattggagatacggtaagtacaagtggtgagaactgctgggattctgaatatattttgaaggtagagtgaacaaattggctattagagctgacgtaggatgttggagaaagagaaaaatcaatgaggacgccaagattttcagcccgaacacaaaaaggatagagtcgcatcggctgtggtggagaatgcggcggcaaggatgtggatggaaacaacaaaacaacataagttcaattttgatatatca

SEQ ID NO: 409 - DNA, 599 bp, Homo sapiens
acacattctacctaaaattttaacaagaataagggcacgaaggcagaaaatgaagagaaaattaggaaacttgcactaacttaggtgagagagatgatagtcgttggattggagatacggtaagtacaagtggtgagaactgctgggattctgaatatattttgaaggtagagtgaacaaattggctattagagctgacgtaggatgttggagaaagagaaaaatcaatgaggacgccaagattttcagcccgaacacaaaaaggatagagtcgcatcggctgtggtggagaatgcggcrgcaaggatgtggatggaaacaacaaaacaacataagttcaattttgatatatcaagttgcacatactagaaactcaagaagagatcattgaatgagcaggcagatgtagggggctgaattctgaagagaggtctgaacaggagataaaatgtgaaggtgtgagtatacagaagtcctttttgtaagccatgaaactggattaaatcaccgaggaagtggaaagagaaaagaatctagtgccagaggttgtgccccagggaattccaaggttcaatggtaaggaataatcagcaaaggag

SEQ ID NO: 410 - DNA, 599 bp, Homo sapiens
tcatatctcagcacctgggatacaaatccctaaattctaagaataaacctgtattgagtgagaaagggcacctgtgccttcctcagacaggtggaaggagtgtcaaggacatggtaggtcccagttgtctcctaggagtgtagacatccttgtctcagggggaatatccattatcttcttcttcctaacccctctgcctagattacattgcatttatgttttactctaaaaccttcttcttgctgccctgactacctaatctttaaactggattggcacttattttccttgccaccaaaycttcttcctcaatgctaaccttaaaaatagcatatgacttgttcacaacgtgcaggtttgtaccctagaacttaaagtataataaaaataaataaataataaaacactaaaaaaatagcatatgactttaggctctgtggatctgaacatttctaaacagctctcagaactccaagttcactgagctacaggctggaggttggattttcctcactcatttccacagacctctcagcatgcattatcccaacagcacagtcaggatcttgccaactgcctttagtactttgaggaagaat

SEQ ID NO: 411 - DNA, 599 bp, Homo sapiens
tattagataatgatataactaaatattttctggtaaaccactttagtcaatgtgataggagtatttgtcatttcctttttctgcatggctttctttttccattcttaataacatatataattgactcattcacgtctagaaagtttgcagattaaatttagtaaataaaattagataaaattaccttcatttgggagttgaacaagctgagctatagttaaggtttgtcaagctctacttcatctaccccgatgtcttttcagtttctaaagcacagcaaatgttgttctgcctcagagyttccacatattgttccctctgcctgtaaacccccttaattttcttcccactctttgatcagccaccccttgttgatcacatctcttaactcaaacagaaacatagacacatgtcacattttttgcataataatacacttttttaggtcttgatttaatatttgtcttagaattatttttaatgtccaattaaaacaagataccaggccccctcctcatgccctgctattctttataactgtaccttgttcttcattgtatttcccataaattgtgacacatcagttagtttattcacgt

SEQ ID NO: 412 - DNA, 599 bp, Homo sapiens
ggctagacacacagatctatcagaaacgttgatggatagaccaagagcccatgtgcttgttgaagcagtaaatcattcagttctctttctcagtgtttttatctgtaaaatgggaaaaaatgctagttctgtaacttatcttattaacttattgtgaagactaaattagttccacttgcaaagtgcagagcttggtacatggtgagcatttaacacactgtagcacatgagtaaaagcatgatctctgaagccaatctgcatggatttgacacttggctgtgttgcttactagttgtgtmttgactgtttaagctttctgtaccttagtttatttatttataagtagagagaaagattagtgacaactacttcataaagccattgttagtattaactgttaactcctgctaagccatcagaacagtgccttgctaatagtaatcatacaatgttagttattattagcttataataatttagtaagcactttagatccaggcatcattcaacttaaatgagtcctataatcaaattgaacctgataattcaagtcccaggaactaggaaacactgggctttctaaaccttatattggtat

SEQ ID NO: 413 - DNA, 599 bp, Homo sapiens
tggggacatttggcagtctctggagatgtttttgattgtcacaactagggagagatattgtcatttttgggtaaaggccatggatgttgttaaatcctacaatgtaccagacagcaactgacaaaaaagaattatccatccctaaagtgctaagtctttactaatgttctagttactcactactcttctagactttatattctggtccattgatctgtttgtttatatcatacagtataaactattacagttttctgatataatatctgtgatggttaatactgagtgtcaacttgatcrgactgaaggatacaaactactgatcctgggtgtgtttgtgagggtgttgccaaaggaaattgacatttgagtcagtgggctgggaaaggcagacccacccttaatctgggtgggcaccatctaatcagctgtcagcatggctagaatataaacaggcagaaaagtgtgaaatgagagactgcctagcctcctggcctacatttctcttgtgctggatgcttcctgcccttgaacgtcagactctgttctctagttttggaacctggaatacctctccttgctcctcagcctgcagatgg

SEQ ID NO: 414 - DNA, 599 bp, Homo sapiens
taattattttatcctttataaatatttttgactctactaagttttgaaaaaattttgttttacagtaaacaattacaagatctccagagaataatttcatatcctttctaatttttatacctcttattttcctttatctaattgtattgtcttataactcacagacaatactatatagtggtggtaataaagagtttccttgttttgtttctggctttaatgagaatgctgttttccaataagcgtaatatatctctaactgttgctattttgtacaaaataaatgttaaattctgacawtttttataaacttttttgagattattgtgatttgtcattcctagggaaatttgttaataggttacccaataataaaacattgttttattcctggaaaactgtagctggtaatgatttgttatacttctaatatgtttctagaatctgtttattaagctttttaaaaatgttttgtggtaaagtatgcataatgataaaaatattaatgcattttaatgcagtaataaaatattattaacattttatctttttatattgtggtaaggtatgcataacataaaatgtatcattttaaccat

SEQ ID NO: 415 - DNA, 599 bp, Homo sapiens
tagctggtaatgatttgttatacttctaatatgtttctagaatctgtttattaagctttttaaaaatgttttgtggtaaagtatgcataatgataaaaatattaatgcattttaatgcagtaataaaatattattaacattttatctttttatattgtggtaaggtatgcataacataaaatgtatcattttaaccatttttaaatgttcagtgatattggttacatttacatttttgagttatccaaacaattttactctgctactcataactttaatagaatctttaagttgtgcatygctgattattttttcaaactatcttatttggagttttatcttttatcttctttctttcttttcttcttgttaactttgatccatcattaaagtattctcttgaaacactttaaaattcttatgtttttacatttaaacaacaggaaaactggaaaagaatgtcacaaatatataacatgttatttgcctaatgctttatcctttccatggctctttcacgttcattatcttatgtgaatctcaagagagtacccataggacatacacaatgactatcataaacagcagtccctattagt

SEQ ID NO: 416 - DNA, 599 bp, Homo sapiens
atcctagatttactagttgtgcaaacttgagatgatcacttaaaatctctgagcatctgtttcatatctttaaaatatcaacaatgatagctttcttttagagttctcagaatttaaagagctaatgcatatgaatactctatataaaaccttctacataggaggtgctaaatagtttgcaaatgtgggtataatttacataggctgtatatctgtacatattaatataaatttaagccaacaaaagaagaatttgcctttcctgatcaatgtcttgccttcattgttatcttcggcctsattataataaacactgaacttatatttcatgatacatttaacactccagtgctttcctaagtattttacacagaaatgattgtttttattttaacaaaatttaaaagctaccatggactgaacatataatacctaccaatcatcatgctaagcattttgttttaatctgaaaatcttcctgtgggtttggtgtttatttgtagattctttttctcttgttttctctctcttccttcctttcttccttccttctgttcttattccctatccccttttatatattctatgtagagtttata

SEQ ID NO: 417 - DNA, 599 bp, Homo sapiens
tcctgtgtcctcctttatggtttaaccatttaagtgaactacggttaatacatttttcatgtttaaactgcttagctaagttgcatataaattatttctcctcaaagttctcagtatacacatccaaaagtttagcttcaaggtaaacaataacataaaaatagtaccactggtttcttggagaacttccaatatttgaagtattaatataaacacatatttattaaaattattgcttcttaatgtgttatgtttcccaatgtgttatgttatttattaaaattattgcttcccaataasgtggcagaacccttcaaggaagcttatgttaaatacataattactttgtttacactggtttcttaagacatttcttttcagtataaaacatagtttcttatcagaatcctcattctctccattctattcagaaaaaacttgaaatttgaagaatagtagaggacaaagatggttgatttattctttgaacatttttccctgatttagcacctggaacagaagtagatgactttgcctttaaacgtccttcatgttttctcactatcctagtttctcgttactttaccactgaggcaaag

SEQ ID NO: 418 - DNA, 599 bp, Homo sapiens
aatacaatcttgagaaagaaaaacaaagctagagatctcatactttctggtttcaaaacatatcataaatctacagtaacaaaaacatggtactgctataaagactcaaatatagaccaatggaacagaatagagagccccaaaataaacccacacatatatggtcaattgatctttgaaaaggatgccagggttacacaatgtgaaaaggagagtctctttttcaaatggtatcagaaaactgggtgtctacatatgaaagaatgaagttgaacccttaccttatataatgtaccaaarttaactcaaaatggattaaaaacctaaacataagacttgaaactataaaactcctagaataaaatttaggaaaaaactttattacgttggacttggcatgatttcttggaaattacattaaaagcactgcaacaatgaacaaattaacaaactgaactacatcaaattttaaaacttctctaccaaaaagaaaacaatcagctaactaaaaaagcctatggaatctgtaaaaatatttacaaagcatatatctgataaggcgttaatattcagaatataaaaacaacacctacaattca

SEQ ID NO: 419 - DNA, 599 bp, Homo sapiens
caaaattcccacctgcacaatctcccttaacctccaaaaacaattttaacctcgaactatgatgggttttattttcctaaaagtgtcatcagagggccttctgtattattttgagggtgcttaagaattcagattttgaacgtccatccagacccactgaactataatttggggaggtaggaattcaggaatcttcatctaaataagcctcccaggttattcttatgctctgttaacatttgagaactttagccaaacccaacaatcacactttccaaaactctgatgatatttgggctyagttctggcaacccaattcatatctactgtcctattcctggtcctcactatctaagcagcaggactgggagacctcattgtatttttactatgcagattttttctcaaacctgcagtcccatgaatgagggttgtttcctactgatatctctttattctaaaagtattgattgggcaactagtgggtgtcaggtcctaatataagtgaggatatagcagtgacaaaagaaaaaacaaacaaaacatgttttcctgaagcttagtttttaataatatagacagccaactaagaaaattga

SEQ ID NO: 420 - DNA, 599 bp, Homo sapiens
atacagcctgcttgcctggcccaccacatgaagtctcttgccatagccttaagatgtcagattccgtgaatggacttggttttctagagccaggagctgggtctggctatgttttctgccccatgtcacttttcctaaattggccattaccaaccacacttagaatagattcacctggatttgaagggttaattgcagtgacaggctaccaccagaagagggtagcagagggttgcagagtaagagctgctgctggcctaggtcttcccctactgaagaatgaagttgtcttcaaacaawtggctaatatagtcagtcaagaggtgagttcactggctaaagcaatggcctgagaagaatgttatttataagagctgggactggtcttgaatttcacatgccttggaagaaatagggagtatagtgtatcagagacgattctagtgcccttagataatatagatccttaaagtatatcccataacatgaatgtctactctactatggcctctgtctattcttcaggttcctctcctgtcattcctacccattcaacccctgttaagttccacccaatgaagaacacaatggcttctaag

SEQ ID NO: 421 - DNA, 599 bp, Homo sapiens
atgctaataaagccattgcttaggatgcttccttattatcaccacttaaattataatcaaaatgagctatttaaatgaaattatctcacagtttgcaagtttctcttcattatacccaggaaccaaatgcatatgtcaaggaaaagcttaggtgaggctttcctatttagcttttagaaatgttgacttccaaggacaacttggtattatagaattgtttaacaactctcaggttatttgcatctatataatggaagtgtgaagcagcctccatatgcaaagagataatgatggttctgkacctacagctagtttgaaaatgttcatttcaataagcatgtcctctgtgactgggtgtggtgtctcacacctgtaatcccagaactttgggagctgaggcgggaggatcatttgaggccgggagttccagactaacccaggcagcatagtgagatcctgtttctataaaacaaaacaaaaaataagtatgttctctgagttgggctgtgattggttgtggcactggggatgaggggaaaggactaaaaataaaggtaacatttgaaccctatccaggaagaatgtatgcttcgaatgaa

SEQ ID NO: 422 - DNA, 599 bp, Homo sapiens
agcttaagaatatttaaggaaactaatacctagtactcatcaagctaaagttcacaatgcctgacatctaataaaatattgacagatgtgaaggaagtggaaaatatatgaccactaataaggagaaaaataaatcaataaaaaacaaaaagacacaggtgacaattagtaaacaagaacataaaagagttaaaatacattccatatggtaaagaaagtaggcaaaaccatgaccactttaaagagaagcatagaagacagaaaaatgcaaaattgaatttctagagagaaaaactaatygcacgagatgaaaactatcctgaagagggtaaggagcagaatagacacactgggaaaaatattggtgaaattgatgcataataaaagaaactgtctaaaatgaaacacataggtaaaagtagttaaaaaatcatagcatcagagagttaaggaaaaaaactctgcctcatattatgcataattggggccatagaaaggcattaaaatacatgaaaaaataatcaccaatacacatcataatgaaattgattaaaaattggtaaagagaaatattaagggtatccagagataaaacagag

SEQ ID NO: 423 - DNA, 599 bp, Homo sapiens
gattgactcttcctttaacaaaagagttctctgtagcatgtggtgctgattgatagcattttacccacaatagaacatccttcaaaattggactcaatcctctgaaatcctgtccctgctttactaagtttatgtaattctctaaatccttagttctcatttcaacagttttcacagtatcttcactgggactgaattccatctcaagaaaccactttgtttgctcattcacaagaagcaactccttatccattcaagttttatcatgacattgtagcaatttagtcacatctttagacyccacttttaattctagttttcttcctatttccataacacatgcagtcatttccttcattgaagtcttgaatccctcaaagtcatctataagggttggagttaacttcttctgaactcctgttaataacgatattttaacatttgcccgtgaatcacagatgttcttaatggtatctagaatgatgaatccttttagaaggtttccaatttacattgcttggatccatcagcccaataactgctatctatggcagctatagccttacaatatttatttcttaaataatgacttgtaggtt

SEQ ID NO: 424 - DNA, 599 bp, Homo sapiens
cttttgttaaaggaagagtcaatcaatgtggcaaagttccttgtcttattttaagaacttgccacagctaccccagccttcagcaactactgccatgattagtcagcagccatcaatattgaggcaaaacttctcaccagcaaaaaattatgactcactaaaggcccagataatcactagcatttttagctatgaagtatttttaatgaacgtatgtactttttaaagacttgaacacttaatagactacagtatcatataaagatatatgtatatgcaatgaaaaaccagaaaatgcgygtgacttactctcatatgacttttttctttattgtggttgtctggaaccaaacctgcagtatctccaaggtacgtctgtattttctattttgttgttcatttcaaatggaccatgtattaaattcctcttgaaatttagttctatggggaaaaaaatgctgtttccaaggctagatccactacacctaaataatctcataagctacatcatatcattttggtggggcttgaagggggatggagcatcaaacaatcaaccaaataaataactttgtagtcttatggtgtccagaaataga

SEQ ID NO: 425 - DNA, 599 bp, Homo sapiens
atgaagtgaattgactgattatgcatactctttactttgggactagacatgccacttgataattaaagtgtcagtgaagtcaagaatatttgtaaaatttgtgtgaagagattattactgtaagatttgttgatttttccatgcaaatggtgtcattaaataatgtgtatttgctacgaagattttatttaatgccacataaatatttacaataacgcatctcaacacaacttcatgggccaaatttttagactaaagaggaaaaccagtgtaagaaattttatgttaacttgtcaagawtgagaaacaaagacccaggatcactttgggcctttgaatggtcatttttatatattttctatgccctttatttcacgaaagctttattcttgcttttatatatgtattttatatattttctatgccctttatttcatgaaagcataagaaggaacagaagaaagtcactggtagcttgatggggatggcattgaatctataaattaccgtgggcagtatggccattttcacaatattgattcttccaatccatgagcatggaatgttctttcttcacagaattgggaaaaactacttta

SEQ ID NO: 426 - DNA, 599 bp, Homo sapiens
ccattacttaataaatacccaaaggaatataaatcatgctgctataaagacacatgcacacatatgtttattgcagcactactcacaatagcaaagacttggaaccaacccaaatgtccaacaatgatagactggattaagaaaatgttgcacatatacaccatggaatactatgcagccacataaaagggtgagttcatgtcctttgtagggacatggaagaagttggaaaccatcattctcagcaaactatctcaaggacaaaaaaccaaacaccacgtgttttcactcgtcggtggraactgaacattgagaacacttggacacaggaaggggaacatcacacactggggcctgttgtggggtggggggagggggaagggatagcattaggagatacacctaaagtaaatgacgagttaatggatgcagcacaccaacatggcacatgtatacatatgtaacaaacctgcacattatgcacatgtaccctagaacttaaagtataataaaaaaaaaatatatatatatatatatatatataaaggacagaatataactataagcagtttgtgtttcttcagtatgatatatagggc

SEQ ID NO: 427 - DNA, 599 bp, Homo sapiens
tactgattcaatcttggcactctttattggtttgttcagatattcaattcctcataacaatcttagtaagttgttggtgtccaggaatttgccttcttcattcaggttttccaatttgttgatatatattcatagtagtccctcatgattctttatatttccatggtataatttataatgactcctttgtcattcctgattttatttatttgggccttctctcttttttccttagtttagcagaaggctggtaaattttgtttatcttttcaaacaacaaaattgttgcttcattgatcrtattagtccattttcatgctgctgataaagacacacccaagactgggcaggttgcaaaggaaagaggtttagtggagaactcacagttccacatggctggggaaacctcagaatcagggcagaacgcaaggaggagcaagtcacatcttacatggataaggcgaagagagcttgtgctggaaaactccctgttacagcaaccatcagatcttgtgagacttactcactttcacaagaacagcacaggaaagacctgcccccatgattcagttacttcccactgggtccctcccacaaca

SEQ ID NO: 428 - DNA, 599 bp, Homo sapiens
gttacagcaaccatcagatcttgtgagacttactcactttcacaagaacagcacaggaaagacctgcccccatgattcagttacttcccactgggtccctcccacaacatgtgggaattgaagatgagatttgggtggggacacaaaccgtatcattgtacatttttagtcatttatacatttagagcttttgctatatcccataggttttgttacgttgtgtttccctttttgtctcaaaaaatttttgtcaattttcttaatttattcattgagctattggttattgcagagcatgtkgtttatttccacgcattgtatagattctaaagttcctcctgttattaatttctaactttagtccactggggtcagagaggatacttaatacaacttgattttttaaacttccttatgacttgttttgtggtctaacatggtctattctgcagattgttccatgtgttgttgagagtaatgtgcattctgaggctgttggatggaatgttctttaaatgtctattagttctattgtgtttagaatgtagtttagcactaatctttctttgttgacattttctctgaatgatctattgcta

SEQ ID NO: 429 - DNA, 599 bp, Homo sapiens
ccctttagtttgtttgagtgggaaggtatttattgctactttatttctgaagacagctttcccaggtgctatactagtggttgacagtgttttctgtttctttgtttcagcaccctaactatatgtccccactccctcctggcctgcaaggtttctgctgagaagtctgttgccaagggtatttgaatactcttatatgtttgcctcttttctttcacatctttcagaatcctgtctttgacctttgagagtctgattataatatgtatgtattttcaaatagcttatgttcaagttaartgattctttattgtttttttttgactctactgttgattgtttctattgtatttttcatttagctcattgtttttctcagcttcaatatcctgttttatattattatctctactgaatttctctgatgagtttctggactattgctttgtgttttcttgaatcaggttttgcctgtgctcattcttctcattgggccttctagcggttttaaccagactgttgtgttgccagagcctgtcaccactgcagccatctaaaccctagagggtgctctggtgctctaagcacaggtttgccac

SEQ ID NO: 430 - DNA, 599 bp, Homo sapiens
tgttatggcagccctagcagatgaatgtgtgactttttatttggacaattatctagacttacaaaaacacataaattcacaaatactatataaaatcttaatgtatttgtattatatcataaatatttctatttgcaaatgttcactcagtttacccttgtaagaggagataactcacaccaatgatgcagttgttgatcaaaaccattttagaaactaattttagttttactgtcggcatctgtctaataactcttattttctgcttccccttgaactgcaagcacatgagtggacatwtgcccaagactgactgatcatgttatctcctgcacctagcactatgattcatctggtcatgaaatgtcttcaaaatagatgctttttttgccttccaagaaacttctcccaattttaataatctatttatttcaaataaataataaatttatttcaaatcaattaataattatttatttcaaattgttttcctataacttacaactgaaatgtaaaagtcttggtcaataccttttcttaaaaaaaaatctttccttcatagtaatatccacaaagattaagtgtggttataaatctta

SEQ ID NO: 431 - DNA, 599 bp, Homo sapiens
attactattttcctcatttggaatatttagaattaaatatcatttattttttccatcaagatggaaggcaaggtgttatagtaaatatgacatattaatttaatattttttatcaagaggattcaaatattagactgggaattagaatatgtgagttttatttctaccttttactccaagaggtaatttatatgtttttttaaactaatttataagggttggtttctaattctaattctatttataactgtgtggtcatggaagggtgatccagatttccaattctataatgttaaatamtccagttctaaaaatatctaagatttataaccacacttaatctttgtggatattactatgaaggaaagatttttttttaagaaaaggtattgaccaagacttttacatttcagttgtaagttataggaaaacaatttgaaataaataattattaattgatttgaaataaatttattatttatttgaaataaatagattattaaaattgggagaagtttcttggaaggcaaaaaaagcatctattttgaagacatttcatgaccagatgaatcatagtgctaggtgcaggagataacatg

SEQ ID NO: 432 - DNA, 599 bp, Homo sapiens
ggaagacataataatcaaataaatataaaatacaattttaggtagtatgattggtgctatgagaaaaattagaacaaaagaaggagccagagagaaataatcatataaattgagaggggattgtcagatacagatattctgaataatggacatctgaataaaggacagaatgagtaatatctgggacataatcattacagacagacagaacagcaagagactgtcctcaatcatcttctcatctagtttccaaataatggagacaataaacacaaatgcagtctaaagagagatcaaagwatacatttactaacagctaagaaaatgttcaattatccctaaatttaggatttctggaatgatgaattagagagtatggatttaaattggtattacaatggaccatcttactctctccttgaatctaaaccttgaagcatactgatggggaccacagtttgatatcacaagtttcatctactttgccaggcacctgctcatgcaccaaattgcctgcaaagtggacagctcaagaatgccaagtgaaattctgagagggaatgacaggttacattatggcaggtcagggaattgatcta

SEQ ID NO: 433 - DNA, 599 bp, Homo sapiens
ttttgctgctgtgaatagtgctgcaataaacatataagtgtctttttggtagaacaatttattttctttggggtacatacctagtaatgggattgctaggttgaatggtagctctgtttaaagttctttgagaaatctccaaactggcagtcagttggggctagagtgtctcagaaagacatgaagagtcctgggggatgggcacttaaggctatgctccatcagagatgctccacgccaaaaaaattaaaaaagcccctggatgctaaatagtagatggatgctaaatattacttgtartgataaattaatgtcctattcccaacaccaagtcttgaaaatgctcattttatatatttgcttcaaatcctattttataataggaagtatttacggataaagtttaagtacctttgcctccatttccagtcttgatgtgaaatccttctctgaggcaaaattgccatgaattttattcttatctttcagtcattttattttacatttgctgctgtgtacctgtaaatcatatacagtatcttaagctttttaaagctaaataaatagcatactatatgtatccctctgaaacttgctta

SEQ ID NO: 434 - DNA, 599 bp, Homo sapiens
gttggttccaagtcttacatatgtaacaaaactgcacgttgtgaacatgtactctagaacttaaagtataataaaataaataaataaatataaggtgagatatatttttacttcttctataatttatacatttacaaatatgtcatttgtggtttttggctttttgcaattaatttatatttttattacgatacggccagaaaacacatctggataattttgcttattcagtattttttaatatttagtttttggctaggttgatggctgtttattaaaacatttcatattttactaaayaaagtttgatgtatcactttctgagagattcattaaaattttgtgttacagttaaggatacttctatatcaatgtccaattttgtagctttattgataggagcaaaaatatttagggaattgtatatttctggtacaaaattgtaagatacttagaatcaaacaacagaaactataattaaaattttgtataaaaataaaatgctactgaatcttaagagcctttaatttgtatggtagagaattattgcttttcttgtcaagaaatgattttatctttaaaaaatatttttggcttaa

SEQ ID NO: 435 - DNA, 599 bp, Homo sapiens
acatgtttaatgtatacaattttaagatgatagatattcatatacactcaggataccatcaccacaatgggggtaaaaaaaaaaatccatcactttcagaagtttcttgatgtccctttgtgtgtatgtgtgtgtttgttttgttattgtggtagaaattcttaatctaagatatgctctcttaatatatgtttaactgtataatactgtattattaactgtaggcactatgttgtatagcagatccttaaaactgtttcatattgcataactgaaacattgagaatgtagaaagatackgccattatgaaaaaacagtatggaggttcctcaaaaagttaaaatagaactaccatacaatctagcaatcctgctactaacaatcctatatatacgaaggaaatgaaatttgtatcttaaagacacacatgtactcccatgtttattgcagcactatccataatagctaagatataaaatcaactgatgtgtccattgacatgcatggataaagaaaatgtggtatatatacacagtggcataccactgagtcttaaaaatgaaacaaatcctgccaattgcaacaacatggatgaact

SEQ ID NO: 436 - DNA, 599 bp, Homo sapiens
tctagaagtcttcataatatctttgtagccttgcattattgattgacatttttacatatgcaggatttatgatgaagctgtgagccacatcaatggcacaaattatattgtatgcagcagtaacagttatacattttataaaacttctgtaaattgttggcatagacctcaaaagtgataaattgagggccaatggtgaaaaagatgggtttcagtaataagcaagcacattatgtctttgttataaaaatctttctatctcccttattttcacctaaaataatcctattcatttgttcytatgaaccttaatatgtacacacagtgagacataaaccatacaatctttaactcatctaaatgtccctaaattatgttaatttttcacaatgctactatagagatcatattttctcattacttctgccctctctgtttcaaacataatcagctacttgaagtcctaaaagaatatgtcatctctatatttggctgccccctttacctgggcaacagtttctgtcccttttttcctctctattttcttacaccaatcttctttcaagctcagttcaggcatcatctctctctaggaagtc

SEQ ID NO: 437 - DNA, 599 bp, Homo sapiens
aaacagctcaattggttatagcttggcatttgccttatttgaacacggtttgaacagttggccacctttgattggccaaaacatggtgtttagcataagagtagattacagtctgtttacacatccagttaggtttcagtttacctgtagggagatacctttaggctgaacttaaaatatgtaaggaggcagcattaagctaaacttaatttaacaggtaaatatctctgtaaacccaaacttagtttggcaatttatgtaagtatttggagaaaggtgagtgagaaataagcatgataytgataatttaggtaaatatgcttttcaaccactgacttgcgtaacaatatttggtgcatctatttgaaaagatatcacttgaagcttaatcactgagattattttcttttcatcataggaggtatgttaattttgctaatttgttagaaattttaaacttttgagagttgaaactacttcttccgaaatctgagtccctatcacaattcacaatttctgattgtctactcaaaacctgttgtcctttgtctatgcctaagcaactcctactgtgcctgaatgtctaattatctctatga

SEQ ID NO: 438 - DNA, 599 bp, Homo sapiens
agagagagaggatagaggggagagagagggagagggagagagagggagagagagggagagagagagagagagagggagagagagagagaaagagagagagagggagagagagagagagagctctttaacgtgagatatccccacaatgaagcaaatcgcccagttatcaaagtgagctatccttaggagttgtcagaaaatgcatcaggattatcagagaaaagtatcagaaagatttttttttctgatacgttgtataaaataaacaaactgaaattcaataacatataaggaattctstctgggctctgaagacaatctctctctgcatattgagttcttcaaacattgtagcctctttatggtctctgagaaataactaccttaaacccataatctttaatacttcctaaactttcttaataagagaagctctattcctgacactacctctcatttgcaaggtcaaatcatcattagttttgtagtctattaactgggtttgcttaggtcaggcattattattactaaccttattgttaatattctaaccataagaattaaactattaatggtgaatagagtttttcactttaaca

SEQ ID NO: 439 - DNA, 599 bp, Homo sapiens
ccacaaatcctaacttactcagcccagcatcattcttctcctcttggcaggtaagatatgctagatatacgatgtcagagtagggaggaaccttaacaatcacttcttcaggcagggtataaacttctcacctgaacactcattgcagcccccatcaaggacagaaatggtgccctgttaagaactctcaatgtatcttgtatttttcctttatagcacactttagatatttgtgtaattatttgtttaatgacttctactaggtttctaaactgcaatgaacagagtacttgattcatyattgcatttcccagaacatagtatggtgcctttaaaaatggcaatagtgcattatgtattttgtgaattgaataaatgagtgaatgaatgaatatctggatgattaaaaaaacaaatgatatacactaatgtctctgaaaaataaaattattattcacaacaggattcatatggtgtacaaattaaaaatgcaaattcctggccttatcccagactaattggtcagaatcttcaggagagagtcccgagaaggcacaatttactaaatgtctatggtgattcttttaatcaggaatgtt

SEQ ID NO: 440 - DNA, 599 bp, Homo sapiens
ttgccactaataagcccttgacactactaaaacatagtaggtgagcagcaaatgctcatttcacctttcttaaactgtggtttgcttgtttctcttcagtcatcttccttccactctgttttctaattttatttttgaaacctgtttctacacagcaatttaatcatagaacaaggttgaagttgataattgtatcaatttcagttcagggcttactgacttgacacacttttttttttttctagagagtattaaaattgaacacattcatgaagagagaaaaaggaaaaagttctgctygacaattaataagtaattaggaaaataatactcatacagttgtggatagcatctggtattacacatctcatgtaattaaaaattaatgtgtatctatctattaagatttcctgtggatgttgcagcttattttggtaatttagacagtcctccaacctttctgggtcaagttaagccacatagttaaataaagcatcataactgactgaaaatcccatgtccaatcctggcagttttttaaacaggaacagaggatataagagctagatgagagtttgcacattatagattaaggtagg

SEQ ID NO: 441 - DNA, 599 bp, Homo sapiens
cacttgtaacttttcattacatgtagtggttttccagcctgtctgtatctgccagcctgtcagtcgctccaaggtggtgatcatttctaaatatttttgtatccacagagcacaggactataatatacacatggaacaggctttataaatacatgctgaatttaacatcaaagaaacgttagctccaatgctaacatacataaaatattaaactctgcatgggtagatttctaggaccattcactgcagtttttttatagcactggattcccaatgccaaggatacaacaatgtcaacartagccacttactcaaatttgggtataatatatcttgtgtcgttctctctggtagttccatatgtatgtctgcgtgtgtgcatgcaaagaaagacaatacaagagagaagatgaagcaaggtgcaacatagctgagtgtgatttaaatgaagttaatatttattggtcagagttttctgttaaatatgttaaaatcaggtaattaatttatttggaaatattgtgttcctaattcatgtctgttgtttgctcatttatttaactcccaaagctttaaattaggggccatgtttatcatca

SEQ ID NO: 442 - DNA, 599 bp, Homo sapiens
gggtcttggctaccactacccatccaccagccctacctagtttccacctcctcaccctctgcccaaggccactggtgggaggaaagaggtcaacatttcactgtcactctccatccctggtggggtccttggtgcaagcaaaaagaggtgtgaggttgggtgttgatcccatgatttgttggttcagagtaaaacagaggccttctctaccgaggctggctgcaccttagcacggtgcacaggtgattacatatagacctctcagccagtattatggactgaattgtgtctcccaaaaaygtgtgtgttgaatccctaatttccagtgtgactatttggaggtaggtctttaaggaagtaattgaggttaagtgaggtcattaacagtggggtcctaatccaacagtactgatgtcctcataagaagacaaaaaagacagagccatctctttctttgtgtatgcgtagagaaacggcaatgtgaagacagagaaaaggtttcctctgcaagacataagagaggcctcagcagataccttgatcttggacttccaaactcatgaactttgagaaaataaatgtctgctgtttaagccacc

SEQ ID NO: 443 - DNA, 599 bp, Homo sapiens
aaaagttaaaggagttaaaaaatataataagttatatatattgttttactgtctcacccactcacacttcctagacctactccactagaaggtaagttacataaagtactcaatacttaaaatgctaatacttattaaaaatgtttaagtcaatgaataaggcaatatggtatagacattaaatcttcatgaccatacttattagactgtaaactctataaaaataaaaactatgtttatcgtgttcagtatgtattcttgtattctggtataatgcctgacacatagacattttaaaayttcggtttgaaaagcagcaacaataaatcaatcattctatgagagttcttttgattacatagtttgttagaaggtgctaagtgcacagataaagcagaaattagtggtgtacagtaaatgtttaataacttgttctcaatggaggagtgggaaagtagggagagatgattctgatttgccaattgttgtggtgtaaatactgacaatggtagatttccatctaatgatgggatactgctgaacattctaatattgagaagggttacacactttgaacttttgctagctagtgctaacca

SEQ ID NO: 444 - DNA, 599 bp, Homo sapiens
tcagccttacccactttagcagtgttgtagagctggttagcactagctagcaaaagttcaaagtgtgtaacccttctcaatattagaatgttcagcagtatcccatcattagatggaaatctaccattgtcagtatttacaccacaacaattggcaaatcagaatcatctctccctactttcccactcctccattgagaacaagttattaaacatttactgtacaccactaatttctgctttatctgtgcacttagcaccttctaacaaactatgtaatcaaaagaactctcatagaatrattgatttattgttgctgcttttcaaaccgaagttttaaaatgtctatgtgtcaggcattataccagaatacaagaatacatactgaacacgataaacatagtttttatttttatagagtttacagtctaataagtatggtcatgaagatttaatgtctataccatattgccttattcattgacttaaacatttttaataagtattagcattttaagtattgagtactttatgtaacttaccttctagtggagtaggtctaggaagtgtgagtgggtgagacagtaaaacaatatatat

SEQ ID NO: 445 - DNA, 599 bp, Homo sapiens
agaagcagaagaagcagcagtagcagctttagtgaaatatatgaatatattcatcatggtaataaatacaggcaataggcaacctaatggtgagtaaagtcagttttttccattaaaagagaaaaattaatttcatttccagaacattttgtattgtggaatttcagatataagtctgtggactgatactattttcctacgttttagtgttttagttttctcctgtttaaaatattctaggcagtgggttctttgaggtaagttcattcatttaactgtaataaggacacagcacatagstaaagcctccctctcattatcccttaagttttctgttcttcatcagagattacatctcatgatcttctctaactcctaaatatttatcctacaaatgttttcatcttgcttgcattttgcattcaaacatcgattcattcttttctgcttatagctgactcagtcatgaaacccatttcactcacataaatttgcattaggaaaacaaaatcacaaacaaacacaacaaaacaaaaacaaacaaaaaaaaactcaatcaagtcacaaatttaccaggagtttggggaagacataggcac

SEQ ID NO: 446 - DNA, 599 bp, Homo sapiens
atggtgagtaaagtcagttttttccattaaaagagaaaaattaatttcatttccagaacattttgtattgtggaatttcagatataagtctgtggactgatactattttcctacgttttagtgttttagttttctcctgtttaaaatattctaggcagtgggttctttgaggtaagttcattcatttaactgtaataaggacacagcacataggtaaagcctccctctcattatcccttaagttttctgttcttcatcagagattacatctcatgatcttctctaactcctaaatatttmtcctacaaatgttttcatcttgcttgcattttgcattcaaacatcgattcattcttttctgcttatagctgactcagtcatgaaacccatttcactcacataaatttgcattaggaaaacaaaatcacaaacaaacacaacaaaacaaaaacaaacaaaaaaaaactcaatcaagtcacaaatttaccaggagtttggggaagacataggcactgacttgtggcaaggcagacaactcaaaatttaacactagacatcgcagctcctaaaaggcctgtgacaccttaacatgtagaaaa

SEQ ID NO: 447 - DNA, 599 bp, Homo sapiens
cttgacttgttctttttttttcctttttgtcattgttttacaccccaacttttattattgtactgtgcagagtggttttactgtttcattcatttccctgttttattcaaaaccataatctcagggtcttacatccagaatcaattgaagcaaggcaagtctcatcctaaagatgaattctgttttccagctatgaaaatagaaattaggtttattcttctatggtaatattcatgattaactttttaaagtaatcaccgtatccaaaacaccatgtcaaacactttgtatggcattaaytcactcaattctaataactatcccctgaggaagattgtatttattattcccacttttaagggaggaaacattatcagagatattcatcttttggttattgattcccattgaaaagtggtggagataatctgcccccagctttatcaagtaccaaagtccaagttctttccattgcactctgataatcaacagtgggttgagagtttctttaaagtggacaataccagtttggtcttttgacataagaaggcaagacataatgccaggtaatctcagagcctatataacaaatctaaggt

SEQ ID NO: 448 - DNA, 599 bp, Homo sapiens
caaaaaaatacaaagatgacttacacagcttatcttactgaaaagctcaccatcaagtgaagaaactccacgtaaatacataaaatggtaattgatataaaatgaggtaggaacccagatacaccagaaatatttcctaaggaggtgaaaatagaattggatcttgaaatatcagtaactgctcactgagcagagaagaggaggagaggcatttccagcagagggaacaacatatgcaatgacacagaggtgtgaaagtgtgagatgagttaccaaaaaatgagaagttctacgtggctrgaatattgagtttgtagtgggaaataacaagagatgtaaggagtttagaaaaatccgtgtataccagtgtctctcaatcttcttgaaggaaaaaaacacattataggcctctaggaatatgttcacaaatccctacagaattaagaaactgaagtttattctaagattctttcaaatcttcacttttaaagagttataaattcattcgtaatacattaaatgttagctgtcatcaccataaaaacccaatgtttaaaaaccataacattcttatcttcaaaagtatgtctgaaaaattt

SEQ ID NO: 449 - DNA, 599 bp, Homo sapiens
agaggcatttccagcagagggaacaacatatgcaatgacacagaggtgtgaaagtgtgagatgagttaccaaaaaatgagaagttctacgtggctagaatattgagtttgtagtgggaaataacaagagatgtaaggagtttagaaaaatccgtgtataccagtgtctctcaatcttcttgaaggaaaaaaacacattataggcctctaggaatatgttcacaaatccctacagaattaagaaactgaagtttattctaagattctttcaaatcttcacttttaaagagttataaattcrttcgtaatacattaaatgttagctgtcatcaccataaaaacccaatgtttaaaaaccataacattcttatcttcaaaagtatgtctgaaaaatttgccttgaaaacatgccattgacaagggtagttgtgataggttattaaacaaggaattgatattatcctacctatacttcaaaagatatgatgttagtggcattagtagaatgcagaaagataatcagtgagctattggggggagtcatggatgagagcttgatctaaagtagtgagaatgaggatgaagatgagttggttagta

SEQ ID NO: 450 - DNA, 599 bp, Homo sapiens
catcaccataaaaacccaatgtttaaaaaccataacattcttatcttcaaaagtatgtctgaaaaatttgccttgaaaacatgccattgacaagggtagttgtgataggttattaaacaaggaattgatattatcctacctatacttcaaaagatatgatgttagtggcattagtagaatgcagaaagataatcagtgagctattggggggagtcatggatgagagcttgatctaaagtagtgagaatgaggatgaagatgagttggttagtatacagaatcagaagctctcggtgactractgtaaggcagcaaggaaagcaacaagttcaggatgattctgacacatctgtgattgatttctaacaattggtccttgtttttgtcctagtaagctatacgtcattttaaaaatgtaatttctcttcatataacagcacttataaagctgatattatgcttccttctctagaaccttaactttttttctgcagcccttcagctctttttgtttcaatgtcactatcaaacacttcactaggaactatttcaccacttcacatgcaaggactttttaaggagcaatctacagagtggta

SEQ ID NO: 451 - DNA, 599 bp, Homo sapiens
agcaatctacagagtggtaaatgagctgtgggctttaatgagaaacaagggaagttttgaaatttttaatgacatttaaacaagaatagtgtgtgtatttgcagagaaaagccagctggaaaactacaatgaagcctccataggtaatctagttttcagtcttcctgcctgagaaatggacttccttagttcatgcaacaacaacaacaaaaaaaaaggtcacacagattcaaaaaataaattcatttgacactaattgtataaactagatagccaaggagatagttaacatggagtgaygcattctagtaattggaaggctaaactatttccctagtatcttcctcactaattctgagggaatcagaaactcagtagtttggtgtaccctgactccgagagaggcagaaggttttgaaagatgattgccagaccaggaaaacctttccgggcctgttttaattaatcccccagatttaccaccttgataaccacattggtattctgatcaggcaataatcctgataataacaataagaattcctatgtgccagacattaatactcctcacatgttatcttatttagtcctcacaacag

SEQ ID NO: 452 - DNA, 599 bp, Homo sapiens
agttaggccaggtgtggtggctcaagcctgtaatcccagcattttgggaggctaaggtggtggatctcttgaggtcaggagtttgagaccagcccagccaatatggagaaatcccatctctactaaaaatacaaaaaaattagcgaggcatggtggcacacacctatagtcccagatactcgggagactgtggcttgagaatcctttgaacccgggaggcagaggtttcagtgagccaagattgcaccgttgcattccagcctgggctacagaccaaaactcctgaagtcttttaactcrtatttgtttaataaatgcataaaatagagttataaattttatttgcaatatgtattgctaatgtgtagtgatatttttcccaagacttatcccaaggaagtgaaatgtctatatgtcttaatgcctgactctatacttagaaaaggtgtattattattttgctccataaagatcttctaggaccctcaattgaattcttggaaaatgacattatcccttttaaatagccattttccacagcagtgagttcaaatcatgaaaaattaaacaaaagctctttagcttggataaatacacat

SEQ ID NO: 453 - DNA, 599 bp, Homo sapiens
gtatcaggtgctcaacaagtactaattttgttttctctgttgctcacttgtaatgtggaaatgcaagtatctattactttcttactttaatatagccaagctttttagatatcaaagaagtctatttatgttatatgaatcaaaggctaatgatcagacttgcaaattgaaaacaggccactccttttacactagagattttactaaacagaaaaatcttttcatagaaacagcatcatttttttctgtccttgaggtaagaggagcttgcaatgaagagaagtataagccatgaaatarcgagtttttataaccatatttctacagttgattataaaatctgctccccctccattaattattacatcttaagaaaagaaatgtgaatatagtaaccagctatagagcagagctgccctgtatgcctgatcataaaaatcatctgaggcatttactaaaattaaagtttcctaggatccagctatacagtgtgtgtgtgtgtgtgtgtgtgtgtgtattatgaaaatatattctagttttggccatgaaacattatcaacatcaacacgttaggactgccaaattggcgtatatgtaaa

SEQ ID NO: 454 - DNA, 599 bp, Homo sapiens
aataaaatagttctacttgtatgaaataaagctagaacttccctgcctaaatcctgtcttttttcactgtttctgttgtaactctggtttacattatgatcttattcacaatttctgcttgcttgatatttcttaattctgaaatttaataatatgcccttcgtcgtgttcccccaacttggttcctgcttacaattcaattttgttatccaaacttctttttctggtcctaactctcagttttgcctgagtgggcactaaaaagttatcttcctcttcctcttcttaatgggcacatgrgagagaacacaaaattctaagagctcatatcttagcgtctaattgatacggattaaaatcccaactctacctgggtagcttggtaaactcagaacttctctgaacctcagttttctttgaatagggataataatttctaaccaacagagttactgtaaagatagaagaacatatgtcacaataaacatttagcatactcaacatttagaaaataattgctcttattaaaatttttcccattattactgttgctgatgcaataaatggaaatatttattggagttctgaatatttttgaa

SEQ ID NO: 455 - DNA, 599 bp, Homo sapiens
gtactatcattaaattcaatgtctgtaggcatgtttgtttagtagagagaatgtcattctgtagatatatagctgtgaaaagccactctgcaacctttgaaaattagaattttctgctcccttagaattctctattaattctattttactattagacatttgcataattattaaaataactcaaaaatattaagcaaagatcaaggtaatttctgagcctttgtaacaaagctgatgaagattctgatgattcaattatcattatacagaaagctagagaactgtgggttaccacagaayattgtgtggcatctgagccatcctctcaagttgctacctatgaagactcatttttccgatcagacaggtctattgactgccattcaattatgcacaaatatatttattggcaaactatgtactgagtgtattttcttaaacattccacatttattcctatttctgggctctgattagaccattcctcaaatctgtagcctacttttcttgacctatctagtttgtacacaaacttcaagactagtttcagttttgacctcctccataaactatgcccaggtcatgtctgtctataacta

SEQ ID NO: 456 - DNA, 599 bp, Homo sapiens
caggtctattgactgccattcaattatgcacaaatatatttattggcaaactatgtactgagtgtattttcttaaacattccacatttattcctatttctgggctctgattagaccattcctcaaatctgtagcctacttttcttgacctatctagtttgtacacaaacttcaagactagtttcagttttgacctcctccataaactatgcccaggtcatgtctgtctataactatcactccaaaatctaattatgaataccatttcccaacttggcattcagtgacctcaggtcggtakcctgaaatcagccatggtgagagtatttacaccatgaaattggcaaatggtacacgtcagggcttttattccaaaagacctagttgttaaaataatggccaacacaccactgctctaatctattaactcacagcacattctttctatgtcccttattttggacacatcaattatctttttatttttatctcctcatctgttaactgttttcttggctgcaggaagcttttttgtttaatgtaatcctgtttgtatattttttattttcgttgcctgtgctttgaggatcatttccaaaa

SEQ ID NO: 457 - DNA, 599 bp, Homo sapiens
tcagtgtatagctattttacttccttgtttaaatttattcctaagaatttttaaaatttttatttttaattaacaactaatggtatatatttagtaagtacaatgtgatgttttgatctatacatttttgtgatgctattgtaaattgattgtttccttcactttttttggatagttcattgttagtgtatggaaatgctactgatttctgtatcttgtttttgtatcctgtaaccttactgaagtcattttttaatttttaatagaattttagcagatttcttagggttttcagtatayacaatcatgtctgcaaaccgggaccatttaatttcttcctttctaatttggatacatttcatttctttttcttagctaattgctctagctaggacttacagtatttgttgattagtcatggtgagagtgggtatccttgtcttcttcctgatatgagacaaaagaagttctactttttgccatgaagtatgatgttagcattgggtttgtcatatatgcaaactttattgtattgaggtacgttccatctatacctaattttttgagagtattatcacaaaaggatgttaaacttgttg

SEQ ID NO: 458 - DNA, 599 bp, Homo sapiens
ttccttgtttaaatttattcctaagaatttttaaaatttttatttttaattaacaactaatggtatatatttagtaagtacaatgtgatgttttgatctatacatttttgtgatgctattgtaaattgattgtttccttcactttttttggatagttcattgttagtgtatggaaatgctactgatttctgtatcttgtttttgtatcctgtaaccttactgaagtcattttttaatttttaatagaattttagcagatttcttagggttttcagtatacacaatcatgtctgcaaaccsggaccatttaatttcttcctttctaatttggatacatttcatttctttttcttagctaattgctctagctaggacttacagtatttgttgattagtcatggtgagagtgggtatccttgtcttcttcctgatatgagacaaaagaagttctactttttgccatgaagtatgatgttagcattgggtttgtcatatatgcaaactttattgtattgaggtacgttccatctatacctaattttttgagagtattatcacaaaaggatgttaaacttgttgaatacttttttctattaata

SEQ ID NO: 459 - DNA, 599 bp, Homo sapiens
tgtgtcaggagtggttaatggcagtgtgggccaatggaagaaaactttttttacatttagaatctgttaattagtatccctgggagttcagataaataatgtagcttttttgagacttattttcttcatcactaaaatgggaatccctaaagaggtcctgaccaactcagaagttatagaaatcaaagaaggtatgcatctaaaatgctctctataaagagtagggagcatagtgtggtaatgaattaaagagtcagagagacctcgactgagatatgagtcagttaaccactcaatcayggagcctctgctctatataatggatatcacataattccacctttgtcagcgtgttggaagtattttaaataacactcaggtgatactgaataataagatgctacaataattatcattctccaacaatttgaataactattcttcaaaaatttgttattctcaaaagtttgtaggttttgcattattccagagcgggcatggcaaaggaatacagtgctttattggggtttagaggtcccaatcagttgatataaatgttacagggtagtattaatgttttagaagattccgttttattg

SEQ ID NO: 460 - DNA, 599 bp, Homo sapiens
ttacatttagaatctgttaattagtatccctgggagttcagataaataatgtagcttttttgagacttattttcttcatcactaaaatgggaatccctaaagaggtcctgaccaactcagaagttatagaaatcaaagaaggtatgcatctaaaatgctctctataaagagtagggagcatagtgtggtaatgaattaaagagtcagagagacctcgactgagatatgagtcagttaaccactcaatcatggagcctctgctctatataatggatatcacataattccacctttgtcagsgtgttggaagtattttaaataacactcaggtgatactgaataataagatgctacaataattatcattctccaacaatttgaataactattcttcaaaaatttgttattctcaaaagtttgtaggttttgcattattccagagcgggcatggcaaaggaatacagtgctttattggggtttagaggtcccaatcagttgatataaatgttacagggtagtattaatgttttagaagattccgttttattgttttatctattcattctacattcattcaataaatacttatttagcatgtg

SEQ ID NO: 461 - DNA, 599 bp, Homo sapiens
gatatttaaaaatattccatttcaaaagcatatgcatgtatattcaaacgcaaatatatctattaatagaggtgagaagggtataaaaatgttggtaatggacccctggttggtagaattatgcataaattttatttcctttgtgttttacaaattttaaaacttttttgcaacagatatgtacatttttgcaataatatcaaaaatagaggctatttccaaaactgcacagataaataagcaaacaaacatacaaaagggtagagagggatattttatcatttgcctatactcttaaarttgcttaaacttgtgtttgtttccttttatctatttttattaaagtaattactggtgactcatattttacagaattatctctctcatagattgattcaataaagagatctactaggcagttgagctccaggtcctgcatttttcctgacatactctgtatttctctctgtccagctccagcttctgaaatgccctctcagcttagcacggcctgcttatggcgagaaccatgtattcaatggctcctccttatttctgcaagtcttcaaattctccttttggtaattctctgcatggaa

SEQ ID NO: 462 - DNA, 599 bp, Homo sapiens
aacaaacatacaaaagggtagagagggatattttatcatttgcctatactcttaaagttgcttaaacttgtgtttgtttccttttatctatttttattaaagtaattactggtgactcatattttacagaattatctctctcatagattgattcaataaagagatctactaggcagttgagctccaggtcctgcatttttcctgacatactctgtatttctctctgtccagctccagcttctgaaatgccctctcagcttagcacggcctgcttatggcgagaaccatgtattcaatggstcctccttatttctgcaagtcttcaaattctccttttggtaattctctgcatggaattaactcttgcaaatccatgtctcacttttgtgtgacctattttgtcttatgctacacattgtccatgttttagcagtagattggaagcttttatgtgcatagccttcccaagaggaaagtaaaaggttggtgctagatggtgcaccttcgataaatgtcagaacagcaaaaaaagggctttagaaatatttccttctttgcacattcccattcaagaatggcttatacagaaaaaaaacaag

SEQ ID NO: 463 - DNA, 599 bp, Homo sapiens
ctaaatactattattctccaactagtttcctcgagggcagatatgtcaacaactaagtaatgtaaagcaaaacgtaactctgcagtaatagtggctcaataggacaggatatttggaagtgagaaaaagagcaccaaagaacggggaccacaggagtagtcctctttaacccctgaatctgcagcacctatcactatgcctagcataagatagtcaatgaatagccgtttgatactattgtttggaatacaattttatgactggtggttctgtgaagaatgagtagattttttacttgtracatttgtgtgtaaaaacagtcaatatatagagaacaaaacaaaaaggtgtagaaatatgtggttgaaagaaacttcagaaaagtcaatagttacaacattacaaaaatactaaaaacaaaactaaggtttattgtgtgcttactacacaccaaattctaagcgcatcacatgaattgactcattcaatcaatagttacagaagaatagactgaaattccgagaggttaagtaaccagtactaaatactgtttcataaagggtttagatggaggcttaatagaaggaaagtgtatattg

SEQ ID NO: 464 - DNA, 599 bp, Homo sapiens
atactttataaactataacacattaacaaagactagctcctgtaaagagctaaagtataaggggcttaaaaaaatattcaatgcttttgttctttactcactgtactctaaatgctctccagtattggcatcacctcactgattctcccttagcgtgtctcttaaatgtgtaggaatctagtttaggtgcaaacctgaaacaatacattccaggaatggccagcctgcctccctccttagctacagattaattacatcttctggagtgacattttggctgtattttccagtgaggcttcycttctactctactccccaagcctccccttggccatcctgctaagaagactgtcctaacccacatgccttcaaaccagacgccttgccaacttggcagcccagaaacagtccaaccagaagggcggcagtaagaaaggtccctggggcttcaggaatgaacagcagattgcttccctgctggtgactgcaaggctgaagcttgctcagaaaggcagaaatctcagggttatgggggtaggtaagcttggaaaccagaaaaatgtcttaaagtctattctagcaatcagatgggatggcag

SEQ ID NO: 465 - DNA, 599 bp, Homo sapiens
gttttaaaacttaattatttagcattatttaaaatcaatggagtgaatgaggtcacagaaattgagtataaacacagaagaaaaggaagcagtctggcataccccatcattaaaaggatgagccgatacagatgagtcagcaaaagcaaagaagacagagggtcattgaccagtgaggaagcaggaaaaaaaggacattaagccacaaaagccatgtagccaaaaaaaaaaaaaaaaaaacagtggaaaaataaatcaggtactcttaagagatccataaaatgaggagagaaaagtacytgttgggtctgaaaacacaaggatgatcatgcccttggcaacagaaaagtttcagtagaatggaagcttcgggagccagactggagcagattctaaacaaaacttttgaaaagttttcacaaaataaggagcatttttaagtgggaagcaggaaagaggatatgaacaagaaaggaggatttgagatttcctttttaagagagaggatatgtattagagaatgattatatgttgttgaaaatgaaaacattcagagaaagagacataggagaaaaaggaaaaaattgccacagcaaggt

SEQ ID NO: 466 - DNA, 599 bp, Homo sapiens
gccgatctcaattaactacaattttaagtatatagtctagaagttcttaacttagttgaaaaaaggggaaacaaacccttggggaatcagtaaggcagttaaaatcagattaaaatacttaaactaatgatgaaacttggagaaacttagtataagaaccaaaaaggcctaaataattaaaagtttaagaaagaaaggcagtttcggtgtttttttctctccttttccaaaaaacatttaatatcattaagtatcaggcaccctgctatgaataattcaatgtgcatccaggattgagakacactgcattaggcagaaattgcactcaaataaaatgagccaggccacactggccacagtagtcaagatgaatctagaaaatgtctcctaacagacctccagacaatgctgggtgtaaatgggaggtctgctattaacttggctttcagtaggaattactgatgggccttcaaagcctcacccctcttacctgtgaagtggtgataacaaaacttaccttactcccttcactgagttgtccatttatttattcaataaatattctatcatgttaagaatcatgacatgtcaagctccag

SEQ ID NO: 467 - DNA, 599 bp, Homo sapiens
agataagtttatagaatattctaagtactttgttgtcagttcaacagtgttgacagcatcttcaccagaaataagtttcatctcaagaaactattttctttgctcatccataagatgcaacccctcattcgttcaaattttattatgggattgaataatttgtttccatcttcagggtctacttgtactccagttctggtgcaatttcccccacatctgcagtaactttctccactgaagtcttgaaccccctcaaagtcatccatgtggtttggattcaccttcttccacactctatgmatgttgatattttggccacctcccatgaaaattaatgttcttaattgtatctaaatagttaatcccttccaaaaggttttctattcattttgctcaaattcatcagaggaatcactgtatgtcggctatacccttatagcatgtatttcttaaataataacaattgaaagtcaaaattactccttaatccatgggctgctgaatggatgttgtgtaggcaggaatgaaagcaatattcatctccttgtgtatctccatcagagctcttgggtgaccttgtcaatgtgcagtaatatttt

SEQ ID NO: 468 - DNA, 599 bp, Homo sapiens; identical to SEQ ID NO: 137

SEQ ID NO: 469 - DNA, 599 bp, Homo sapiens
cagaggaattgctgaatctagtgttaccaatttattttgagataacacaaaactttatgcttcgacaatgttattcctgaacactttaaatcctgaaagtgcattataatccttaatttattaccagtttattatcacaggaatcaaattctgaggatctttaaagtcatggtcattttgcttaacataggccattttgtacatggcaaccatgtgaagagcagtagaatcagaagaagaaaaaaaaaggttttgagacatgactctatcaactgactgtaaggtgacctgggaaattcrctctacatccctgaatctcagtttattcacctgaaatactgggaccagaacacattaaagaattatttagaatgatacattaatgagcctagtacagtgtaacacagggtaaacatccagcagttttggaatcatttttggaagtttcttgctagggttaccaagaaaatttgtagaaatcttgaacttaagtgtagttaataataatagctattataatgtttattgctctatgatgacgatagtaatataaaaccccaggattagaattagatttgagctctaaataatcttcctgc

SEQ ID NO: 470 - DNA, 599 bp, Homo sapiens
gatacttcagatgacatcaatatgacaggtagaattaactagttgaaaaggaggacatggttatgaatttgggccatattgttagaataatgcaagccactgataaattttaagcaggttataatcaaatttgaattttcaaaagattattgcagcttcagtatgcagaaaaattttagtagttggaaagcacatgcagaaagccagtgaaaatgctattataagagtctgaggaagaggtaaaggtagtttaagttgcagtggtggtggcaggaagggagacagaaagcatgattaatytgatattattttgaaggcaaaatttacagaacttggaaaattatttatatatcagatgaaggagattaggaatggctttacgctttctggcctgtgaaaatgatgaatggtagtataattcactcatataataacaagaggagcaaagaaagacatggaggaaagcttatggttttgttatacagtgaaatgaagttatatgggacggcaaggtgaatgtctcttgagaagttggatacacagcctgaaactcaaaagcggtgtctggccctcagatcctaatgcgagagacatcagca

SEQ ID NO: 471 - DNA, 599 bp, Homo sapiens
aaggtgaatgtctcttgagaagttggatacacagcctgaaactcaaaagcggtgtctggccctcagatcctaatgcgagagacatcagcatataaattattgactcagctaatttttaaaaccccatggaagggtgatttttattctgttttacaggtaagtaaattgagactcagaaagataaagtgattaattttaaactacaaagctagtaagtgtcaaatactgaatccaaaacttcatcactccatcactgcacagagaagagggttccctttaacccctacattgtcttctacrcatatgctccacctctttgttccttccctagtgtcccaatggatactttttactacaaaggcaacaaaatttcctggatgagccaggggagaagaggctcaatgagtcatattagaagcttgaaggtatttatttccaattaaatttaaagaaacaatttacttgaaatgctttagagtaattttcacacggctttgaatttacaaagttaattttagagaagtttgactttaaaataagcaacagcagatacttgggaggctgaggcaggaggttctcttaagcccaggagtgtgagg

SEQ ID NO: 472 - DNA, 599 bp, Homo sapiens
caaactagtttctttgctgccttgtgacatttggcacaaacgtccatttattataattatatctatttatatatgtcttatttttcaagcttgtcacaaactctcaagagcaagtattatgtcttacttgttttatctttcttcaactcatttattaaaatatcacttattcattgagatacactacctgtaggcggggagttggggggcactctgtctcatttaccttttcaccctcagcacctggcaggtaaacaaatcatttcgagagtaatgttgttctgatgaagatagaaaggsatcatggagaaaagacttcctacatctctgtgaagtcagggaatgcaccccagaagactgtcccactgagtaaaagaatgaattagactctgagcatataatgttgatggcaatccatcctaggcagaaaagaacagcatgcacacaggtatacacagccaaacagagctattgaacaaattcatgaatgatagaagtgtcatcaaattccattggcagaatggggcactgcagttactgtgaacagtgtttccccttctcatttttgaaaccaaatttccaggtcttttgtaatgtat

SEQ ID NO: 473 - DNA, 599 bp, Homo sapiens
tctcctaaatagattctatagttatatttacataaaatttaaacatctctccttaactgctacgtgccttaaatatgtagttgattgtgagaatgagatttgtttttcattaaaacaaatctttccttcccatttccacagctgccattcagggacattccttcaacatatcttctgggtccaataacacattcagtcattcagtaaacatgttcattctctcatatgtgtttcagacactgtaccagattctagaattatatcaatgatgaaagcacactattggccttcaaggagccrttttaatagaaacaataggttaattgttgatgtatagggaagtttaatttatgtaatacttatctagccgtattttatttttttactatgcattatctgattatggatttcctaaatatatagttatatcatctattaattgcatcaggtacttttagtagcatgctttctaattgtatcagtaaaaagactttttttttattttggaaaattaatagaataaaccaaaaataaaaatcaactgtaatcttattgttagattgatcacttgtgctgttttagtctcctgtgtacttttt

SEQ ID NO: 474 - DNA, 599 bp, Homo sapiens
ccaacccccgaagccttaggacagagggacggtgcccatgcgccaaaatctgtttcaagatttaagactgtgacctctgtgaatacagagattgttcaaatgccctagggttcttgggaggctactaagattgctgagcatttgccaacattactattaatgtctgtcaaataattaaaagactggattagtgaatggttaatgatataacggtgttctattgataatcggaatagttacaacatcctttcacagaaaaagatggaaaaatcacagctaacatttaattgagtacacackatataccaggccctgaatcacttacggatgttatctataaaattcaaacgttccaacaagaggggtattattttcccatttttctgatgaagaaactgaggctttggagtattaggtgtaactttcccaagctcttacagttaataagtagtagagctggccttcaaacccaggtgtctactccaaaggactgtgaaaggatgaagatgatggtgatcgtaacaatggtggtaacaataaaaacaatgggatgtctttttatttcagacccagactcttttcaagactacattaagtcc

SEQ ID NO: 475 - DNA, 599 bp, Homo sapiens
catagcattctgatagtttataaatgcatttaaaaataaattgtctgaaaaatttgagctcttggaaacacctttatctgtataagactgacctaaaaatcaagaccacataattttcaaatgattactttttatttagtcccattttagatttcaaagagaggcaaatatcatcctactttacacatgtcctaaccacacacatgtcctaaccacacattttggttaattttaaaatctacaataaggaaaaatttatgattatttctgaagttatcttattaattatcagaaattcckaacagttactaaacttggtaatagagcagcagtcatcttctgactctcattagttgtcagacttaagctagcatgtctaatttctgatactgactgcaggaaaatctagcaagaagtggggagtagcacccatggacacagtggaagtctgtgggaggaaacgataaataccagggagtatatcctcaactcagtacataaccagcaaaaaagatttcaagaagatcattaacaatggtgtctgaggattagagtcaattatagatgggaattgaagctagttaaatgaagagcaagaa

SEQ ID NO: 476 - DNA, 599 bp, Homo sapiens
tctgctgtactgaacagattgttccttaaggaccaaagcattcatttcccccagctactgttactgttggctgctgattgctcatagctgagtccctcttcaagaactgtgctcacagaagggagtctctggcctgagtctcttgcctggggcagctcaaatgcaatgactagacaatgtgggaaaacaaatactgaagctcctggctttgatgtgggacagttctaaaagaccatcccaactgcagatagctgttggatcacctgagacttctggggaacctgaagactactggtgccmaaagtagtcttaggaagcattctctgaaataggattttgctgctggattatccaccagttaattggcaatgaaaactccatcactagtagtagttgaaatacggatagctcctagcatgacatagtgggcaattgttaaaaactttatcagtattaactagaatgggcttatagaagaattgaatgcacctgtaactaaagcataatttagggatttgggaagttcaaaggaagtggcttttgttgagtgctattgatacattgatgctgaaaaatgacaatgaaagtttaggggtgac

SEQ ID NO: 477 - DNA, 599 bp, Homo sapiens
tcttactatcatcctaaatatcaattctcagtgaagtaatgtcttttgtagcaacatggatggaggttattattctaagtgaaataactcaggaacaaaccaaataccatatgctctcactaataaatgacagctaagccatatgctatgcaaaggcatacagagtggtgtaatggacatttgaaactcagaaggtggaaggatgggaacagggtgagggataaaaaagttacctgttgggtataacgtatactatttgggtgaggggtacacttaaaacccagacttcaccattatacratttatccatgtaatcaaaaactacttgtacccctaaaaattattgaaaaaaattactttctcatccaaacttccacaacttaataaatggcacctgtacccaggttatttctcatgcaataatcttggaagtcttctcagatatagtttcccacacatctaatctatttgcagtagtcctatttctgcctgcaatatgcaatcactttggcatgtctctacttcattctaacgtgccctcactgtcacatcacagcaatctctctatttcagttctagactaagtcataatcttctgt

SEQ ID NO: 478 - DNA, 599 bp, Homo sapiens
cacatatcttagcctggtattctaatttcaactcatgtttcgtccattgatatctgaaaataatttttttgaaccaaactatttttatgaattattccagtattgcctattattaataacttttctttatgatctatcatgctatggaagctctgatggaaatgttcaccatctccatctaagaacattgtcttttagagtcactgaaagatttatcacatcagcatgccttcaactcgtactgaggatttttctataagtagttcccacttctcagtgtcaccatgtttaccagtttgrgttctgtatttccttttagttgaagtattacctagtttaatgcaacctggacttctttcagagctatcagcaatcttcactttttggaagtccttgacttactgaagtgagggctagatcttattgtttgttaagcataaaatatgtttattctgtttttacttttctctatttgcttagatttaatttttgatgagtttcactgtcattcttccgtttaatatttatcctattcttactatggaccagacattgggaatataatttctcaaaattaatgcagaatttatcttcatgaa

SEQ ID NO: 479 - DNA, 599 bp, Homo sapiens
taatgttttaactctgccctgccaatcacctctgttttgtgttagggaactgacagaggaatatacagtaaacaatatgcaaaggggagagaaggttctattaacagcgatgtcagtgactaactttagtttggggttctctgctaaagctaggttggtgcacaagacttaagcaatagtctcaagtgaaactttcaatggttagaaaaaaatgactccaaatctggggcacttaccacccttagtctatcaaattgtgaaaggaaggcaaagagagaaaagaagtacaacaaagatgamagcaacctcattgacagtctagcatatgtatggcatagaagaaatggaagagagaatgagaatatgggataaggtcctgcatccacttttctcccagggacaaaagacaatgagagatgaaggtactaagaggcacgcaaactgtgtcggggtgcccagtatcacctaggtttactaattctctaaggatactcaagatctcagtatagttttactcacagctatgatttactacaatgaaagaatacaaagcgaaatcagcaaagggataaagtgtgtggggtgaagtccaggggaaa

SEQ ID NO: 480 - DNA, 599 bp, Homo sapiens
ctctgccctgccaatcacctctgttttgtgttagggaactgacagaggaatatacagtaaacaatatgcaaaggggagagaaggttctattaacagcgatgtcagtgactaactttagtttggggttctctgctaaagctaggttggtgcacaagacttaagcaatagtctcaagtgaaactttcaatggttagaaaaaaatgactccaaatctggggcacttaccacccttagtctatcaaattgtgaaaggaaggcaaagagagaaaagaagtacaacaaagatgaaagcaacctcawtgacagtctagcatatgtatggcatagaagaaatggaagagagaatgagaatatgggataaggtcctgcatccacttttctcccagggacaaaagacaatgagagatgaaggtactaagaggcacgcaaactgtgtcggggtgcccagtatcacctaggtttactaattctctaaggatactcaagatctcagtatagttttactcacagctatgatttactacaatgaaagaatacaaagcgaaatcagcaaagggataaagtgtgtggggtgaagtccaggggaaatcaggcacaag

SEQ ID NO: 481 - DNA, 599 bp, Homo sapiens
ttaattttctgtgcaaatatatatatatttaatattttaaaattatataaatatttaatgttcctatcttactgagcacaattatgagatttaaaaataataacaaaaaacaaaccaaaataaaaccaaaatcccaaacccagcatgttaaatttaaatatcagaatcctatcattcccaattacatatcctgctgtaaggaaaattacagaagcgttgagtgacttagaaattagaacactttcaaagtgtttcaaaggagcagttcttaaatactacttttttattatcttcacatgkaagactgatccttctttaggatgaaaacaacactatgctgttctcaaaatcctagagaaaattcttgattattattgccaaaagtgcgtaattcccaatgaaaagagattttaagtatgtttgaacaaattttatttgtttcagttgtaagcaccgtgtcatgttctaaagcaataaaggttttcctacatactatgactttatacatactatgactttataggaataagattatatataaatatatgcatattttcaattatttatgaatattgaatttgatttagtgctttctcttc

SEQ ID NO: 482 - DNA, 599 bp, Homo sapiens
tcagagcatacctttagcacttatttacttgtagcgtttgtatcttttgataaataatttagaatgcaattacaaaattatttaaaaaaggaaagaagcacattatttagacctcttctctgctagacctttagcccatgaagaacctcagttggtgcctgacttacagtgcatgctcagaaatgttactgagaaattacttattttaggtgttcacattttgtgtctacatatttaaatagtaattcctacatgttgtgagaaaactcatttgtaccttctatttcaagcatcatgtgwgtgtgtttgtgtgtattgtgtgtctgcattggtgtggtgttttccttggtaggtggaggaggggataatactccaagatctataatatggggagtatgaatgaatgacaactactcttgaagatatgttcatgtgtggtagtgtgcacatccatgccctacatctactctttaaatatatatctatatatacacacacacacgtatagatagatgattgatagattacttttagataaatactgaaaactaaaattcaaacaaatctaaagattaaaaattcaaacaagatatagataa

SEQ ID NO: 483 - DNA, 599 bp, Homo sapiens
atattttggataatcagaatttcatcagttaaagcatgttttattcactttttcactcatctaacaaacttttattgagtacctatcatttgtcacatactgtgaacaaaacaaattattatcaccaaggcagctctgtttattaagcacacacaaaaaatgccaggacaacaaaagaacttcaatgccattatagctacttgaaaagaatttagaatgtaattacaaaattatttaaaaagggaaagaagcacattatttctagggatttaaagaaattaatggtttgtcaaaatttgstatttattttccttagaagtatgcatattaatcttagaaaaacaaattccttctaaaaagaagaattaaagggcaaacttagagtatatccataccaactagaaagatatggagcatacatctccctactgaaaagtttgtttaaatcaagcgaatgtcctttattaccatggtaactcaataataatacatggtaatgcatagtaatacatggtaactgaatagtaacaatataatagtaacaatgttgctatttaaactattggtttcagataaacatgagaggctctcaaattagt